(19) United States
(12) Patent Application Publication
(10) Pub. No.: US 2020/0392498 A1
(43) Pub. Date: Dec. 17, 2020

(54) POLYNUCLEOTIDE CONSTRUCTS HAVING BIOREVERSIBLE AND NON-BIOREVERSIBLE GROUPS

(71) Applicant: Solstice Biologics, Ltd., Dublin (IE)

(72) Inventors: Curt W. BRADSHAW, San Diego, CA (US); Sukumar SAKAMURI, San Diego, CA (US); Laxman ELTEPU, San Diego, CA (US); Son LAM, San Diego, CA (US); Dingguo LIU, San Diego, CA (US); Bryan MEADE, San Diego, CA (US); Giuseppe Dello IACONO, Oceanside, CA (US); Joseph STOCK, Mooresville, IN (US); Bin LIU, San Diego, CA (US)

(21) Appl. No.: 16/780,204

(22) Filed: Feb. 3, 2020

Related U.S. Application Data
(63) Continuation of application No. 15/315,608, filed on Dec. 1, 2016, now abandoned.
(60) Provisional application No. 62/009,123, filed on Jun. 6, 2014.

(51) Int. Cl. C12N 15/113; C07H 21/04; C12N 15/85; C07H 19/10; C07H 19/20; C07H 21/02

(52) U.S. Cl. CPC C12N 15/113; C07H 21/04; C12N 15/85; C07H 19/10; C07H 19/20; C07H 21/02; C12N 2330/30; C12N 2310/31; C12N 2310/32; C12N 2310/346; C12N 2310/3513; C12N 2310/3515; C12N 2310/14

(57) ABSTRACT

The invention features a hybridized polynucleotide construct containing a passenger strand, a guide strand loadable into a RISC complex, and (i) a 3′-terminal or an internucleotide non-bioreversible group in the guide strand; or (ii) a 5′-terminal, a 3′-terminal, or an internucleotide non-bioreversible group in the passenger strand, and a 5′-terminal, a 3′-terminal, or an internucleotide disulfide bioreversible group in the guide strand or the passenger strand. The invention also features methods of delivering a polynucleotide to a cell using the hybridized polynucleotide construct. The invention further features methods of reducing the expression of a polypeptide in a cell using the hybridized polynucleotide construct.

### SEQUENCE LISTING

This application contains a Sequence Listing which has been submitted electronically in ASCII format and is hereby incorporated by reference in its entirety. Said ASCII copy, created on Jul. 27, 2020 is named 50925-010003_Sequence_Listing_07.27.20_ST25.txt and is 40,659 bytes in size.

### FIELD OF THE INVENTION

This invention relates to compositions and methods for transfecting cells.

### BACKGROUND

Nucleic acid delivery to cells both in vitro and in vivo has been performed using various recombinant viral vectors, lipid delivery systems, and electroporation. Such techniques have sought to treat various diseases and disorders by reducing or inhibiting gene expression, providing genetic constructs for gene therapy or to study various biological systems.

Polyanionic polymers such as polynucleotides do not readily diffuse across cell membranes. To overcome this problem for cultured cells, cationic lipids are typically combined with anionic polynucleotides to assist uptake. Unfortunately, this complex is generally toxic to cells, which means that both the exposure time and concentration of cationic lipid must be carefully controlled to insure transfection of viable cells.

The discovery of RNA interference (RNAi) as a cellular mechanism that selectively degrades mRNAs allows for both the targeted manipulation of cellular phenotypes in cell culture and the potential for development of directed therapeutics (Behlke, Mol. Ther. 13, 644-670, 2006; Xie et al., Drug Discov. Today 11, 67-73, 2006). However, because of their size and negative (anionic) charged nature, siRNAs are macromolecules with no ability to enter cells. Indeed, siRNAs are 25× in excess of Lipinski's “Rule of 5s” for cellular delivery of membrane diffusible molecules that generally limits size to less than 500 Da. Consequently, in the absence of a delivery vehicle or transfection agent, naked siRNAs do not enter cells, even at millimolar concentrations (Barquinero et al., Gene Ther. 11 Suppl 1, S3-9, 2004). Significant attention has been focused on the use of cationic lipids that both condense the siRNA and punch holes in the cellular membrane to solve the siRNA delivery problem. Although widely used, transfection reagents fail to achieve efficient delivery into many cell types, especially primary cells and hematopoietic cell lineages (T and B cells, macrophage). Moreover, lipofection reagents often result in varying degrees of cytotoxicity ranging from mild in tumor cells to high in primary cells.

Accordingly, there is a need for polynucleotide constructs with increased ability to transfect cells.

### SUMMARY OF THE INVENTION

In general, the invention provides hybridized polynucleotides having a non-bioreversible group or a combination of a non-bioreversible group and a bioreversible group. In particular, the invention features hybridized polynucleotide constructs having a guide and a passenger strand, where the guide strand includes a non-bioreversible group.

In a first aspect, the invention provides a hybridized polynucleotide construct including a passenger strand, a guide strand loadable into a RISC complex, and

(i) a 3′-terminal or an internucleotide non-bioreversible group in the guide strand; or

(ii) a 5′-terminal, a 3′-terminal, or an internucleotide non-bioreversible group in the passenger strand, and a 5′-terminal, a 3′-terminal, or an internucleotide disulfide bioreversible group in the guide strand or the passenger strand.

In particular embodiments, the hybridized polynucleotide construct includes at least one disulfide bioreversible group.

In some embodiments, the disulfide bioreversible group includes -S-S-(Link A)-B,

where

Link A is a divalent or a trivalent linker including an sp³-hybridized carbon atom bonded to B and a carbon atom bonded to -S-S-, where, when Link A is a trivalent linker, the third valency of Link A combines with -S-S- to form optionally substituted C₃₋₉ heterocyclylene, and

B is a 5′-terminal phosphorus (V) group, a 3′-terminal phosphorus (V) group, or an internucleotide phosphorus (V) group.

In certain embodiments, the hybridized polynucleotide construct includes a passenger strand and a guide strand loadable into a RISC complex, where each of the passenger strand and the guide strand has the structure according to the following formula:

5′-D-(Nuc-E)ₙ-Nuc-F, or a salt thereof,

where

each n is independently an integer from 10 to 150,

each Nuc is independently a nucleoside; and

D of the guide strand is hydroxyl, phosphate, or a disulfide bioreversible group;

D of the passenger strand is H, hydroxyl, optionally substituted C₁₋₆ alkoxy, a protected hydroxyl group, phosphate, diphosphate, triphosphate, tetraphosphate, pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, a non-bioreversible group, or a disulfide bioreversible group;

each E is independently phosphate, phosphorothioate, a non-bioreversible group, or a disulfide bioreversible group;

each F is independently H, hydroxyl, optionally substituted C₁₋₆ alkoxy, a protected hydroxyl group, a monophosphate, a diphosphate, a triphosphate, a tetraphosphate, a pentaphosphate, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, a non-bioreversible group, or a disulfide bioreversible group;

for example, where at least one of the disulfide bioreversible groups includes -S-S-(Link A)-B,
where
Link A is independently a divalent or a trivalent linker including sp³-hybridized carbon atom bonded to B and a carbon atom bonded to -S-S-, where, when Link A is a trivalent linker, the third valency of Link A combines with -S-S- to form optionally substituted C₃₋₉ heterocyclylene; and
B is independently a 5′-terminal phosphorus (V) group, a 3′-terminal phosphorus ( ) group, or an internucleotide phosphorus (V) group;
   where the hybridized polynucleotide construct includes at least one non-bioreversible group in the guide strand, or the hybridized polynucleotide construct includes the disulfide bioreversible group and at least one non-bioreversible group.

In particular embodiments, the disulfide bioreversible group has the following structure:

(R¹)_{q}-(Link C)—S—S-(Link A)-B,

where
each q is independently an integer from 1 to 10;
each Link C is independently a bond or a multivalent linker having a molecular weight of from 12 Da to 10000 Da; and
each R¹ is independently H, azido, a polypeptide, a carbohydrate, a neutral organic polymer, a positively charged polymer, a therapeutic agent, a targeting moiety, or an endosomal escape moiety.

In some embodiments, the hybridized polynucleotide construct further contains a second passenger or a second guide strand (e.g., the hybridized polynucleotide construct contains two passenger strands and two guide strands), where Link C is a multivalent linker further bonded to -S-S-(Link A)-B of the second passenger or the second guide strand (e.g., Link C is bonded to two guide strands or to two passenger strands).

In other embodiments, Link C includes one or more monomers, where each of the monomers is independently optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, where m is 0, 1, or 2. In yet other embodiments, Link C includes one or more monomers, where each of the monomers is independently optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, where m is 0, 1, or 2. In still other embodiments, Link C includes one or more monomers, where each of the monomers is independently optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; O; or S(O)ₘ, where m is 0, 1, or 2.

In some embodiments, Link C includes 1 to 500 of the monomers (e.g., 1 to 300 of the monomers, 1 to 200 of the monomers, 1 to 150 of the monomers, or 1 to 100 of the monomers). In certain embodiments, Link C includes one or more C₁₋₆ alkyleneoxy groups (e.g., fewer than 100 C₁₋₆ alkyleneoxy groups). In particular embodiments, Link C includes one or more poly(alkylene oxide) (e.g., polyethylene oxide, polypropylene oxide, poly(trimethylene oxide), polybutylene oxide, poly(tetramethylene oxide), and diblock or triblock co-polymers thereof (e.g., the poly(alkylene oxide) is polyethylene oxide).

In particular embodiments, Link C includes one or more groups independently selected from the group consisting of

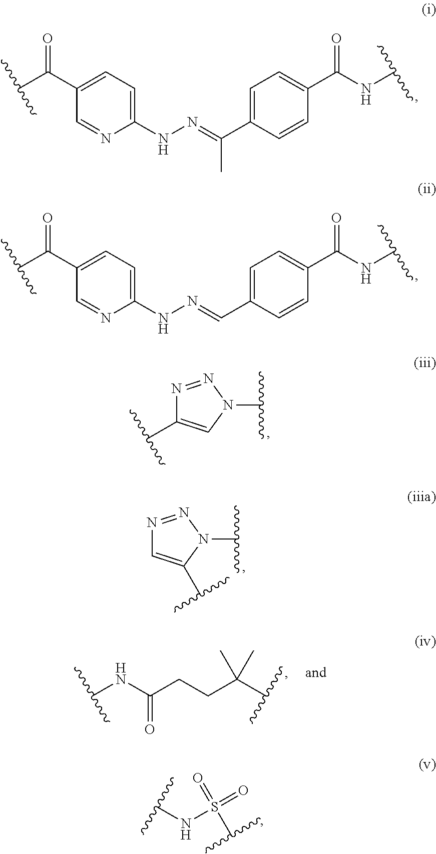

and a combination thereof.

In further embodiments, the hybridized polynucleotide constructs further includes a second passenger strand or a second guide strand (e.g., the hybridized polynucleotide construct contains two passenger strands and two guide strands), where the passenger strand or the guide strand is covalently linked to the second passenger strand or the second guide strand by the non-bioreversible group (e.g., two passenger strands or two guide strands are covalently linked by the non-bioreversible group).

In certain embodiments, Link A includes 1, 2, or 3 monomers independently selected from the group consisting of optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; O; or S(O)ₘ, where each m is independently 0, 1, or 2. In other embodiments, Link A includes 1, 2, or 3 monomers independently selected from the group consisting of optionally substituted C₁₋₆ alkylene; optionally substituted C₂₋₆ alkenylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; O; or S(O)ₘ, where each m is independently 0, 1, or 2. In yet other embodiments, Link A includes 1, 2, or 3 monomers independently selected from the group consisting of optionally substituted C₁₋₆ alkylene; optionally substituted C₆₋₁₄ arylene; optionally substituted C₁₋₉ heteroarylene having 1 to 4 heteroatoms selected from N, O, and S; or O.

In some embodiments, Link A includes 2 or 3 monomers, one of the monomers having the structure:

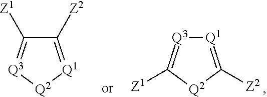

where

Z¹ is a bond to -S-S-;

Z² is a bond to another monomer of Link A;

Q¹ is N or CR²;

Q² is O, S, NR³, or —C(R⁵)═C(R⁶)—;

Q³ is N or C bonded to R⁴;

each of R², R³, R⁴, R⁵, and R⁶ is independently H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁-heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl; or R⁵ and R⁶, together with the atoms to which each is attached, combine to form a cyclic group selected from the group consisting of C aryl, C₂₋₇ heteroaryl, and C₂₋₇ heterocyclyl, where the cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

In certain embodiments, Q¹ is CR². In particular embodiments, R² is H, halo, or C₁₋₆ alkyl. In other embodiments, Q² is O or —C(R⁵)═C(R⁶)—. In yet other embodiments, Q² is —C(R⁵)═C(R⁶)—. Instill other embodiments, R⁵ is H, halo, or C₁₋₆ alkyl. In some embodiments, R⁶ is is H, halo, or C₁₋₆ alkyl.

In still other embodiments, R⁵ and R⁶, together with the atoms to which each is attached, combine to form C₂₋₅ heteroaryl optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl. In certain embodiments, the C₂₋₅ heteroaryl includes two nitrogen atoms (e.g., the C₂-heteroaryl is optionally substituted with C₁₋₆ alkyl).

In particular embodiments, Q² is O. In certain embodiments, Q³ is CR⁴. In some embodiments, R⁴ is H, halo, or C₁₋₆ alkyl.

In other embodiments Link A and -S-S- combine to form a structure:

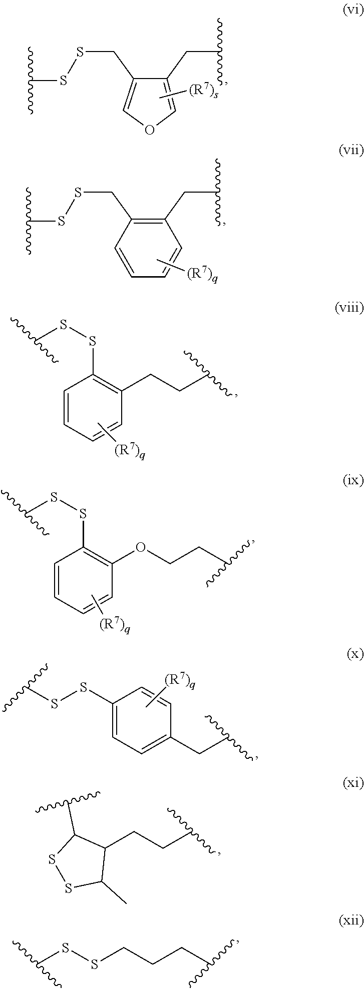

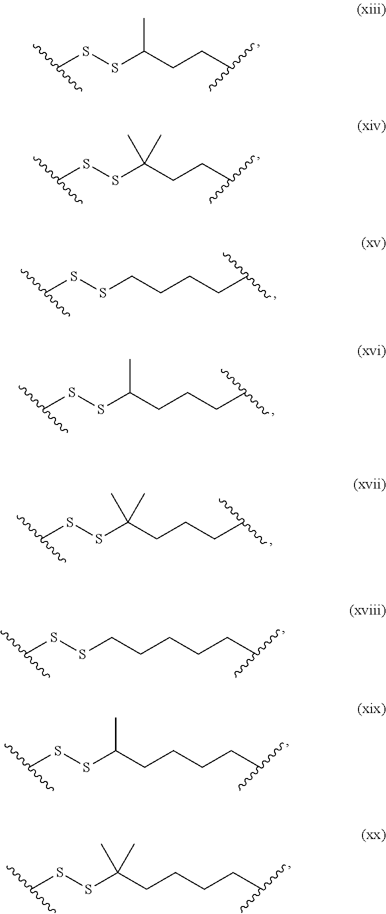

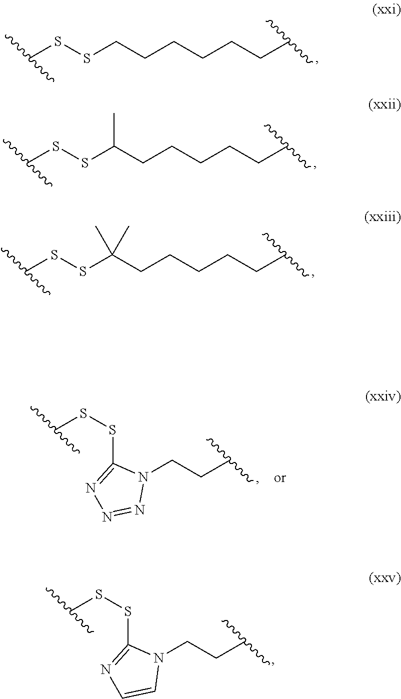

where

each R⁷ is independently C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{d}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl; or two adjacent R⁷ groups, together with the atoms to which each the R⁷ is attached combine to form a cyclic group selected from the group consisting of C aryl, C₂₋₅ heterocyclyl, or C₂₋₅ heteroaryl, where the cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)SO₂R^{D}, where q is an integer from zero to four and where R is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4; and

s is 0, 1, or 2.

In yet other embodiments, R⁷ is halo or optionally substituted C₁₋₆ alkyl. In still other embodiments, s is 0 or 1 (e.g., s is 0). In certain embodiments, q is 0, 1, or 2 (e.g., q is 0 or 1).

In particular embodiments, two adjacent R⁷ groups, together with the atoms to which each the R⁷ is attached combine to form C₂₋₅ heteroaryl optionally substituted with 1, 2, or 3 C₁₋₆ alkyl groups.

In some embodiments, Link A and -S-S- combine to form a structure:

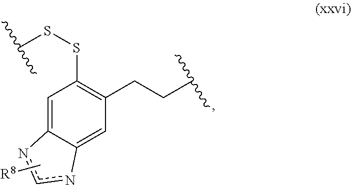

where the dotted lines represent one and only one double bond, and

R⁸ is attached to the nitrogen atom having a vacant valency and is H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-14-alkyl; —(CH₂)SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

In certain embodiments, R⁸ is H or C₁₋₆ alkyl.

In other embodiments, at least one of the disulfide bioreversible groups includes one or more monomers, where each of the monomers is independently optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, where m is 0, 1, or 2. In yet other embodiments, at least one of the bioreversible group includes one or more monomers, where each of the monomers is independently optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, where m is 0, 1, or 2. In still other embodiments, at least one of the bioreversible groups includes one or more monomers, where each of the monomers is independently optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; O; or S(O)ₘ, where m is 0, 1, or 2. In particular embodiments, at least one of the monomers is S(O)ₘ, and m is 2.

In certain embodiments, at least one of the bioreversible groups includes 2 to 500 of the monomers (e.g., 2 to 300 of the monomers, 2 to 200 of the monomers, 2 to 150 of the monomers, or 2 to 100 of the monomers). In some embodiments, at least one of the bioreversible groups includes one or more C₁₋₆ alkyleneoxy groups (e.g., at least one of the bioreversible groups includes fewer than 100 C₁₋₆ alkyleneoxy groups). In particular embodiments, at least one of the bioreversible groups includes one or more poly(alkylene oxide) (e.g., polyethylene oxide, polypropylene oxide, poly(trimethylene oxide), polybutylene oxide, poly(tetramethylene oxide), and diblock or triblock co-polymers thereof). In other embodiments, the poly(alkylene oxide) is polyethylene oxide.

In further embodiments, at least one of the non-bioreversible groups includes one or more auxiliary moiety, each of the one or more auxiliary moiety is independently a polypeptide, a carbohydrate, a neutral organic polymer, a positively charged polymer, a therapeutic agent, a targeting moiety, and an endosomal escape moiety.

In some embodiments, at least one of the non-bioreversible group includes a carbohydrate (e.g., the carbohydrate is mannose, N-acetyl galactosamine, or D-glucitol).

In particular embodiments, at least one of the non-bioreversible group includes a targeting moiety (e.g., the targeting moiety is a folate ligand, the targeting moiety is a prostate specific membrane antigen (PSMA), the targeting moiety is an endoplasmic reticulum targeting group, or the targeting moiety is an albumin-binding group).

In other embodiments, at least one of the non-bioreversible group includes a polypeptide (e.g., the polypeptide is a cell penetrating peptide, or the polypeptide is an endosomal escape moiety).

In yet other embodiments, at least one of the bioreversible group includes a carbohydrate (e.g., the carbohydrate is mannose, N-acetyl galactosamine, or D-glucitol). In particular embodiments, at least one R¹ is a carbohydrate (e.g., the carbohydrate is mannose, N-acetyl galactosamine, or D-glucitol).

In still other embodiments, at least one of the bioreversible group includes a targeting moiety (e.g., the targeting moiety is a folate ligand, the targeting moiety is a prostate specific membrane antigen (PSMA), the targeting moiety is an endoplasmic reticulum targeting group, or the targeting moiety is an albumin-binding group). In some embodiments, at least one R¹ is a targeting moiety (e.g., the targeting moiety is a folate ligand, the targeting moiety is a prostate specific membrane antigen (PSMA), the targeting moiety is an endoplasmic reticulum targeting group, or the targeting moiety is an albumin-binding group).

In particular embodiments, at least one of the bioreversible group includes a polypeptide (e.g., the polypeptide is a cell penetrating peptide, the polypeptide is an endosomal escape moiety, or the guide strand includes the non-bioreversible group). In some embodiments, at least one R¹ is a polypeptide (e.g., the polypeptide is a cell penetrating peptide, the polypeptide is an endosomal escape moiety, or the guide strand includes the non-bioreversible group).

In other embodiments, at least one of the bioreversible group includes a polypeptide (e.g., the polypeptide is a cell penetrating peptide, or the polypeptide is an endosomal escape moiety). In certain other embodiments, at least one R¹ is a polypeptide (e.g., the polypeptide is a cell penetrating peptide, or the polypeptide is an endosomal escape moiety).

In other embodiments, at least one R¹ is azido, a polypeptide, a carbohydrate, a targeting moiety, or an endosomal escape moiety In certain embodiments, one of the non-bioreversible group connects the second nucleoside and the third nucleoside of the guide strand. In particular embodiments, one of the non-bioreversible group connects the fifth nucleoside and the sixth nucleoside of the guide strand. In other embodiments, one of the non-bioreversible group connects the seventeenth nucleoside and the eighteenth nucleoside of the guide strand. In yet other embodiments, one of the non-bioreversible group is a 3′-terminal group of the guide strand.

In particular embodiments, the guide strand includes from 1 to 5 of the non-bioreversible groups (e.g., the guide strand includes 1 the non-bioreversible group).

In some embodiments, the passenger strand includes at least one of the non-bioreversible group (e.g., the passenger strand includes 1 to 5 of the non-bioreversible groups (e.g., 1 the non-bioreversible group)).

In other embodiments, the non-bioreversible group connects two nucleosides of passenger strand, where the nucleosides are disposed at least one nucleoside away from the natural RISC-mediated cleavage site in the 5′-direction. In yet other embodiments, the non-bioreversible group connects the first and the second nucleosides of the passenger strand. In still other embodiments, the guide strand includes at least one of the disulfide bioreversible group.

In certain embodiments, the passenger strand includes at least one of the disulfide bioreversible group. In particular embodiments, the disulfide bioreversible group connects two consecutive nucleosides selected from the three 5′-terminal nucleosides of the guide strand (e.g., B is an internucleotide phosphorus ( ) group connecting two consecutive nucleotides selected from the three 5′-terminal nucleotides of the guide strand). In some embodiments, In particular embodiments, the disulfide bioreversible group connects two consecutive nucleosides selected from the three 3′-terminal nucleosides of the guide strand.

In other embodiments, the bioreversible group is a 5′-terminal group of the passenger strand (e.g., D of the passenger strand is the disulfide bioreversible group). In certain other embodiments, the bioreversible group is a 5′-terminal group of the guide strand (e.g., D of the guide strand is the disulfide bioreversible group). In yet other embodiments, the bioreversible group is a 3′-terminal group of the guide strand (e.g., F of the guide strand is the disulfide bioreversible group). In still other embodiments, the bioreversible group is a 3′-terminal group of the passenger strand (e.g., F of the passenger strand is the disulfide bioreversible group).

In particular embodiments, the disulfide bioreversible group connects two consecutive nucleosides selected from the three 5′-terminal nucleosides of the passenger strand (e.g., B is an internucleotide phosphorus ( ) group connecting two consecutive nucleotides selected from the three 5′-terminal nucleotides of the passenger strand).

In some embodiments, the disulfide bioreversible group connects two consecutive nucleosides selected from the three 3′-terminal nucleosides of the passenger strand (e.g., B is an internucleotide phosphorus (V) group connecting two consecutive nucleosides selected from the three 3′-terminal nucleosides of the passenger strand).

In other embodiments, the non-bioreversible group is a 5′-terminal group of the passenger strand (e.g., D of the passenger strand is the non-bioreversible group). In yet other embodiments, the non-bioreversible group is a 3′-terminal group of the guide strand (e.g., F of the guide strand is the non-bioreversible group). In still other embodiments, the non-bioreversible group is a 3′-terminal group of the passenger strand (e.g., F of the passenger strand is the non-bioreversible group).

In certain embodiments, the non-bioreversible group includes one or more monomers, each of the monomers is independently optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; O; or S(O)ₘ, where m is 0, 1, or 2. In particular embodiments, each of the one or more monomers is independently optionally substituted C₁₋₆ alkylene; optionally substituted C₂₋₆ alkenylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; O; or S(O)ₘ, where m is 0, 1, or 2. In other embodiments, each of the one or more monomers is independently optionally substituted C₁₋₆ alkylene; optionally substituted C₆₋₁₄ arylene; optionally substituted C₁-heteroarylene having 1 to 4 heteroatoms selected from N, O, and S; optionally substituted N; O; or S(O)ₘ, where m is 0, 1, or 2. In yet other embodiments, at least one of the monomers is S(O)ₘ, and m is 0 or 2 (e.g., m is 2).

In particular embodiments, the non-bioreversible group includes independently from 1 to 200 of the monomers. In some embodiments, the non-bioreversible group includes independently from 1 to 150 of the monomers. In other embodiments, the non-bioreversible group includes independently from 1 to 100 of the monomers. In yet other embodiments, the non-bioreversible group includes independently from 1 to 3 of the monomers. In still other embodiments, the non-bioreversible group includes independently 1 the monomer.

In some embodiments, the non-bioreversible group is independently a phosphate or a phosphorothioate substituted with a substituent selected independently from the group consisting of optionally substituted C₃₋₆ alkyl; optionally substituted C₃₋₆ alkenyl; optionally substituted C₃₋₆ alkynyl; optionally substituted C₃₋₈ cycloalkyl; optionally substituted C₃₋₈ cycloalkenyl; optionally substituted (C₃-cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O; optionally substituted C₁₋₉ heterocyclyl having 1 to 4 heteroatoms selected from N, O, and S, where the heterocyclyl does not include an S-S bond; and optionally substituted (C₁₋₉ heterocyclyl)-C₁₋₄-alkyl having 1 to 4 heteroatoms selected from N, O, and S, where the heterocyclyl does not include an S-S bond.

In certain embodiments, the shortest chain of atoms connecting -S-S- to an internucleotide phosphorus (V) group, a 5′-terminal group, or a 3′-terminal group is 3. In other embodiments, the longest chain of atoms connecting -S-S- to an internucleotide phosphorus (V) group, a 5′-terminal group, or a 3′-terminal group is 6. In yet other embodiments, the at least one disulfide bioreversible group includes independently at least one bulky group proximal to the disulfide.

In other embodiments, the guide strand includes 19 or more nucleosides (e.g., n of the guide strand is 17 or greater). In yet other embodiments, the guide strand includes fewer than 100 nucleosides (e.g., n of the guide strand is 98 or less). In still other embodiments, the guide strand includes fewer than 50 nucleosides (e.g., n of the guide strand is 48 or less). In particular embodiments, the guide strand includes fewer than 32 nucleosides (e.g., n of the guide strand is 30 or less). In certain embodiments, the passenger strand includes 19 or more nucleosides.

In other embodiments, the passenger strand includes 19 or more nucleosides (e.g., n of the passenger strand is 17 or greater). In yet other embodiments, the passenger strand includes fewer than 100 nucleosides (e.g., n of the passenger strand is 98 or less). In still other embodiments, the passenger strand includes fewer than 50 nucleosides (e.g., n of the passenger strand is 48 or less). In particular embodiments, the passenger strand includes fewer than 32 nucleosides (e.g., n of the passenger strand is 30 or less). In certain embodiments, the passenger strand includes 19 or more nucleosides.

In a second aspect, the invention provides a method of delivering a polynucleotide construct to a cell including contacting the cell with the hybridized polynucleotide construct of any embodiment the above aspect.

In a third aspect aspect, the invention provides a method of reducing the expression of a polypeptide in a cell including contacting the cell with the hybridized polynucleotide construct of any embodiment of the first aspect.

In particular embodiments, the bioreversibel or non-bioreversible group of any of the above aspects is a group of formula (II) or

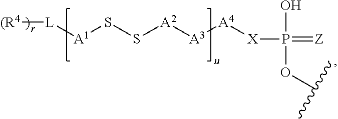

or a salt thereof,

where u is 0 or 1;

A¹ is a bond or a linker containing or being one or more of optionally substituted N; 0; S; optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A¹ includes one or more of optionally substituted N, O, and S, the optionally substituted N, O, or S is not directly bonded to the disulfide; and each A² is independently selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with -S-S-, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;

L is absent or a conjugating group including or consisting of one or more conjugating moieties;

each R⁴ is independently hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof; and

r is independently an integer from 1 to 10.

For the non-bioreversible group, u is 0.

In some embodiments, the bioreversible group is a group of formula (II) or a salt thereof, where u is 1.

In other embodiments, the bioreversible group is a group of formula (II) or a salt thereof, where u is 0.

In particular embodiments, when the bioreversible group is a group of formula

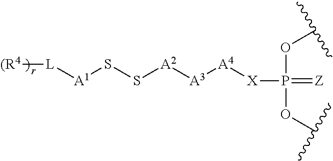

or a salt thereof in which A², A³, and A⁴ combine to form C₄₋₅ alkylene.

In particular embodiments, when the bioreversible group is a group of formula

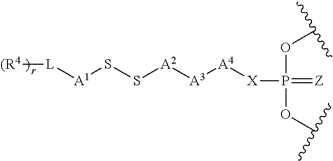

or a salt thereof in which A², A³, and A⁴ combine to form C₄₋₅ alkylene.

In other embodiments, when the bioreversible group is a group of formula

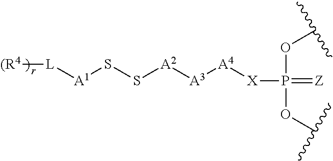

or a salt thereof, the group -A²-A³-A⁴-X- does not contain a phosphate, an amide, an ester, or an alkenylene.

In certain embodiments, each X is O. In particular embodiments, each Z is O.

In certain embodiments of any aspect of the invention, all nucleosides are ribonucleosides, e.g., where the 2′ position of each ribonucleotide is substituted with either F, —OMe, or —O-Et-O-Me.

### Definitions

The term “about,” as used herein, represents a value that is ±10% of the recited value.

The term “activated carbonyl,” as used herein, represents a functional group having the formula of —C(O)R^{A} where R^{A} is halogen, optionally substituted C₁₋₆ alkoxy, optionally substituted C₆₋₁₀ aryloxy, optionally substituted C₂₋₉ heteroaryloxy (e.g., -OBt), optionally substituted C₂-C₉ heterocyclyloxy (e.g., —OSu), optionally substituted pyridinium (e.g., 4-dimethylaminopyridinium), or —N(OMe)Me.

The term “activated phosphorus center,” as used herein, represents a trivalent phosphorus (III) or a pentavalent phosphorus (V) center, in which at least one of the substituents is a halogen, optionally substituted C₁₋₆ alkoxy, optionally substituted C₆₋₁₀ aryloxy, phosphate, diphosphate, triphosphate, tetraphosphate, optionally substituted pyridinium (e.g., 4-dimethylaminopyridinium), or optionally substituted ammonium.

The term “activated silicon center,” as used herein, represents a tetrasubstituted silicon center, in which at least one of the substituents is a halogen, optionally substituted C₁₋₆ alkoxy, or amino.

The term “activated sulfur center,” as used herein, represents a tetravalent sulfur where at least one of the substituents is a halogen, optionally substituted C₁₋₆ alkoxy, optionally substituted C₆₋₁₀ aryloxy, phosphate, diphosphate, triphosphate, tetraphosphate, optionally substituted pyridinium (e.g., 4-dimethylaminopyridinium), or optionally substituted ammonium.

The term “alkanoyl,” as used herein, represents a hydrogen or an alkyl group (e.g., a haloalkyl group) that is attached to the parent molecular group through a carbonyl group and is exemplified by formyl (i.e., a carboxaldehyde group), acetyl, propionyl, butyryl, isobutyryl, and the like. Exemplary unsubstituted alkanoyl groups include from 1 to 7 carbons. In some embodiments, the alkyl group is further substituted with 1, 2, 3, or 4 substituents as described herein.

The term “(C_{x1-y1} aryl)-C_{x2-y2}-alkyl,” as used herein, represents an aryl group of x1 to y1 carbon atoms attached to the parent molecular group through an alkylene group of x2 to y2 carbon atoms. Exemplary unsubstituted (C_{x1-y1} aryl)-C_{x2-y2}-alkyl groups are from 7 to 16 carbons. In some embodiments, the alkylene and the aryl each can be further substituted with 1, 2, 3, or 4 substituent groups as defined herein for the respective groups. Other groups followed by “alkyl” are defined in the same manner, where “alkyl” refers to a C₁₋₆ alkylene, unless otherwise noted, and the attached chemical structure is as defined herein.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted with 1, 2, 3, or 4 substituent groups selected, independently, from the group consisting of aryl, cycloalkyl, heterocyclyl (e.g., heteroaryl), as defined herein, and the substituent groups described for alkyl. In addition, when an alkenyl group is present in a bioreversible group of the invention it may be substituted with a thioester or disulfide group that is bound to a conjugating moiety, a hydrophilic functional group, or an auxiliary moiety as defined herein.

The term “alkenylene,” as used herein, refers to a straight or branched chain alkenyl group with one hydrogen removed, thereby rendering this group divalent. Non-limiting examples of the alkenylene groups include ethen-1,1-diyl; ethen-1,2-diyl; prop-1-en-1,1-diyl, prop-2-en-1,1-diyl; prop-1-en-1,2-diyl, prop-1-en-1,3-diyl; prop-2-en-1,1-diyl; prop-2-en-1,2-diyl; but-1-en-1,1-diyl; but-1-en-1,2-diyl; but-1-en-1,3-diyl; but-1-en-1,4-diyl; but-2-en-1,1-diyl; but-2-en-1,2-diyl; but-2-en-1,3-diyl; but-2-en-1,4-diyl; but-2-en-2,3-diyl; but-3-en-1,1-diyl; but-3-en-1,2-diyl; but-3-en-1,3-diyl; but-3-en-2,3-diyl; buta-1,2-dien-1,1-diyl; buta-1,2-dien-1,3-diyl; buta-1,2-dien-1,4-diyl; buta-1,3-dien-1,1-diyl; buta-1,3-dien-1,2-diyl; buta-1,3-dien-1,3-diyl; buta-1,3-dien-1,4-diyl; buta-1,3-dien-2,3-diyl; buta-2,3-dien-1,1-diyl; and buta-2,3-dien-1,2-diyl. The alkenylene group may be unsubstituted or substituted (e.g., optionally substituted alkenylene) as described for alkenyl groups.

The term “alkoxy,” as used herein, represents a chemical substituent of formula —OR, where R is a C₁₋₆ alkyl group, unless otherwise specified. In some embodiments, the alkyl group can be further substituted with 1, 2, 3, or 4 substituent groups as defined herein.

The term “alkyl,” as used herein, refers to an acyclic straight or branched chain saturated hydrocarbon group having from 1 to 12 carbons, unless otherwise specified. Alkyl groups are exemplified by methyl; ethyl; n- and iso-propyl; n-, sec-, iso- and tert-butyl; neopentyl, and the like, and may be optionally substituted with one, two, three, or, in the case of alkyl groups of two carbons or more, four substituents independently selected from the group consisting of: (1) alkoxy; (2) alkylsulfinyl; (3) amino; (4) arylalkoxy; (5) (arylalkyl)aza; (6) azido; (7) halo; (8) (heterocyclyl)oxy; (9) (heterocyclyl)aza; (10) hydroxy; (11) nitro; (12) oxo; (13) aryloxy; (14) sulfide; (15) thioalkoxy; (16) thiol; (17) —CO₂R^{A}, where R^{A} is selected from the group consisting of (a) alkyl, (b) aryl, (c) hydrogen, and (d) arylalkyl; (18) —C(O)NR^{B}R^{C}, where each of R^{B} and R^{C} is, independently, selected from the group consisting of (a)hydrogen, (b)alkyl, (c) aryl, and (d) aryl-alkylene; (19) —SO₂R^{D}, where R^{D} is selected from the group consisting of (a)alkyl, (b) aryl, and (c) aryl-alkylene; (20) —SO₂NR^{E}R^{F}, where each of R^{E} and R^{F} is, independently, selected from the group consisting of (a) hydrogen, (b) alkyl, (c) aryl and (d) arylalkyl; (21) silyl; (22) cyano; and (23) —S(O)R^{H} where R^{H} is selected from the group consisting of (a)hydrogen, (b)alkyl, (c)aryl, and (d) arylalkyl. In some embodiments, each of these groups can be further substituted as described herein. In certain embodiments, the alkyl carbon atom bonding to the parent molecular group is not oxo-substituted.

The term “alkylene,” as used herein, refers to a saturated divalent, trivalent, or tetravalent hydrocarbon group derived from a straight or branched chain saturated hydrocarbon by the removal of at least two hydrogen atoms. Alkylene can be trivalent if bonded to one aza group that is not an optional substituent; alkylene can be trivalent or tetravalent if bonded to two aza groups that are not optional substituents. The valency of alkylene defined herein does not include the optional substituents. Non-limiting examples of the alkylene group include methylene, ethane-1,2-diyl, ethane-1,1-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, and butane-2,2-diyl, butane-2,3-diyl. The term “C_{x-y} alkylene” represents alkylene groups having between x and y carbons. Exemplary values for x are 1, 2, 3, 4, 5, and 6, and exemplary values for y are 2, 3, 4, 5, 6, 7, 8, 9, 10, 11, and 12. In some embodiments, the alkylene can be further substituted with 1, 2, 3, or 4 substituent groups as defined herein for an alkyl group. Similarly, the suffix “ene” designates a divalent radical of the corresponding monovalent radical as defined herein. For example, alkenylene, alkynylene, arylene, aryl alkylene, cycloalkylene, cycloalkyl alkylene, cycloalkenylene, heteroarylene, heteroaryl alkylene, heterocyclylene, and heterocyclyl alkylene are divalent forms of alkenyl, alkynyl, aryl, aryl alkyl, cycloalkyl, cycloalkyl alkyl cycloalkenyl, heteroaryl, heteroaryl alkyl, heterocyclyl, and heterocyclyl alkyl. For aryl alkylene, cycloalkyl alkylene, heteroaryl alkylene, and heterocyclyl alkylene, the two valences in the group may be located in the acyclic portion only or one in the cyclic portion and one in the acyclic portion. In addition, when an alkyl or alkylene, alkenyl or alkenylene, or alkynyl or alkynylene group is present in a bioreversible or a non-bioeversible group, it may be substituted with an ester, thioester, or disulfide group that is bound to a conjugating moiety, a hydrophilic functional group, or an auxiliary moiety as defined herein. For example, the alkylene group of an aryl-C₁-alkylene or a heterocyclyl-C₁-alkylene can be further substituted with an oxo group to afford the respective aryloyl and (heterocyclyl)oyl substituent group.

The term “alkyleneoxy,” as used herein, refers to a divalent group —R—O—, in which R is alkylene.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. Alkynyl groups may be optionally substituted with 1, 2, 3, or 4 substituent groups that are selected, independently, from aryl, alkenyl, cycloalkyl, heterocyclyl (e.g., heteroaryl), as defined herein, and the substituent groups described for alkyl.

The term “alkynylene,” as used herein, refers to a straight-chain or branched-chain divalent substituent including one or two carbon-carbon triple bonds and containing only C and H when unsubstituted. Non-limiting examples of the alkenylene groups include ethyn-1,2-diyl; prop-1-yn-1,3-diyl; prop-2-yn-1,1-diyl; but-1-yn-1,3-diyl; but-1-yn-1,4-diyl; but-2-yn-1,1-diyl; but-2-yn-1,4-diyl; but-3-yn-1,1-diyl; but-3-yn-1,2-diyl; but-3-yn-2,2-diyl; and buta-1,3-diyn-1,4-diyl. The alkynylene group may be unsubstituted or substituted (e.g., optionally substituted alkynylene) as described for alkynyl groups.

The term “amino,” as used herein, represents —N(R^{N1})₂ or —N(R^{N1})C(NR^{N1})N(R^{N1})₂ where each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, aryl-alkyl, cycloalkyl, cycloalkylalkyl, heterocyclyl (e.g., heteroaryl), heterocyclylalkyl (e.g., heteroarylalkyl), or two R^{N1} combine to form a heterocyclyl, and where each R^{N2} is, independently, H, alkyl, or aryl. In one embodiment, amino is —NH₂, or —NHR^{N1}, where R^{N1} is independently, OH, NO₂, NH₂, NR^{N2}₂, SO₂OR^{N2}, SO₂R^{N2}, SOR^{N2}, alkyl, or aryl, and each R^{N2} can be H, alkyl, or aryl. Each R^{N1} group may be independently unsubstituted or substituted as described herein. In addition, when an amino group is present in a bioreversible group of the invention it may be substituted with an ester, thioester, or disulfide group that is bound to a conjugating moiety, a hydrophilic functional group, or an auxiliary moiety as defined herein.

The term “antibody,” as used herein, is used in the broadest sense and specifically covers, for example, single monoclonal antibodies, antibody compositions with polyepitopic specificity, single chain antibodies, and fragments of antibodies (e.g., antigen binding fragment or Fc region). “Antibody” as used herein includes intact immunoglobulin or antibody molecules, polyclonal antibodies, multispecific antibodies (i.e., bispecific antibodies formed from at least two intact antibodies) and immunoglobulin fragments (such as Fab, F(ab′)₂, or Fv), so long as they recognize antigens and/or exhibit any of the desired agonistic or antagonistic properties described herein. Antibodies or fragments may be humanized, human, or chimeric.

The term “aryl,” as used herein, represents a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl, and the like, and may be optionally substituted with one, two, three, four, or five substituents independently selected from the group consisting of: (1) alkanoyl (e.g., formyl, acetyl, and the like); (2) alkyl (e.g., alkoxyalkyl, alkylsulfinylalkyl, aminoalkyl, azidoalkyl, acylalkyl, haloalkyl (e.g., perfluoroalkyl), hydroxyalkyl, nitroalkyl, orthioalkoxyalkyl); (3) alkenyl; (4) alkynyl; (5) alkoxy (e.g., perfluoroalkoxy); (6) alkylsulfinyl; (7) aryl; (8) amino; (9) arylalkyl; (10) azido; (11) cycloalkyl; (12) cycloalkylalkyl; (13) cycloalkenyl; (14) cycloalkenylalkyl; (15) halo; (16) heterocyclyl (e.g., heteroaryl); (17) (heterocyclyl)oxy; (18) (heterocyclyl)aza; (19) hydroxy; (20) nitro; (21) thioalkoxy; (22) —(CH₂)_{q}CO₂R^{A}, where q is an integer from zero to four, and R^{A} is selected from the group consisting of (a) alkyl, (b) aryl, (c) hydrogen, and (d) arylalkyl; (23) —(CH₂)_{q}CONR^{B}R^{C}, where q is an integer from zero to four and where R^{B} and R^{C} are independently selected from the group consisting of (a) hydrogen, (b) alkyl, (c) aryl, and (d) arylalkyl; (24) —(CH₂)_{q}SO₂R^{D}, where q is an integer from zero to four and where R^{D} is selected from the group consisting of (a) alkyl, (b) aryl, and (c) arylalkyl; (25) —(CH₂)SO₂NR^{E}R^{F}, where q is an integer from zero to four and where each of R^{E} and R^{F} is, independently, selected from the group consisting of (a) hydrogen, (b) alkyl, (c) aryl, and (d) arylalkyl; (26) thiol; (27) aryloxy; (28) cycloalkoxy; (29) arylalkoxy; (30) heterocyclylalkyl (e.g., heteroarylalkyl); (31) silyl; (32) cyano; and (33) —S(O)R^{H} where R^{H} is selected from the group consisting of (a) hydrogen, (b) alkyl, (c) aryl, and (d) arylalkyl. In some embodiments, each of these groups can be further substituted as described herein. In addition, when an aryl group is present in a bioreversible group of the invention it may be substituted with an ester, thioester, or disulfide group that is bound to a conjugating moiety, a hydrophilic functional group, or an auxiliary moiety as defined herein.

The term “aryl alkyl,” as used herein, represents an alkyl group substituted with an aryl group. The aryl and alkyl portions may be substituted as the individual groups as described herein.

The term “auxiliary moiety” refers to any moiety, including, but not limited to, a small molecule, a polypeptide, a carbohydrate, a neutral organic polymer, a positively charged polymer, a therapeutic agent, a targeting moiety, an endosomal escape moiety, and any combination thereof, which can be conjugated to a nucleotide construct disclosed herein. Generally, but not always the case, an “auxiliary moiety” is linked or attached to a nucleotide construct disclosed herein by forming one or more covalent bonds to one or more conjugating groups present on a disulfide bioreversible group or on a non-bioreversible group. However, in alternative embodiments an “auxiliary moiety” may be linked or attached to a nucleotide construct disclosed herein by forming one or more covalent bonds to any portion of the nucleotide construct in addition to conjugating groups present on a disulfide bioreversible group, such as to the 2′, 3′, or 5′ positions of a nucleotide sugar molecule, or on any portion of a nucleobase. Although the name for a particular auxiliary moiety may imply a free molecule, it will be understood that such a free molecule is attached to a nucleotide construct. One skilled in the art will readily understand appropriate points of attachment of a particular auxiliary moiety to a nucleotide construct.

The term “aza,” as used herein, represents a divalent —N(RN1)- group or a trivalent —N=group. The aza group may be unsubstituted, where RN1 is H or absent, or substituted, where RN1 is as defined for “amino.” Aza may also be referred to as “N,” e.g., “optionally substituted N.” Two aza groups may be connected to form “diaza.”

The term “azido,” as used herein, represents an N₃ group.

The term “bioreversible group,” as used herein, represents a moiety including a functional group that can be actively cleaved intracellularly, e.g., via the action of one or more intracellular enzymes (e.g., an intracellar reductase) or passively cleaved intracellularly, such as by exposing the group to the intracellular environment or a condition present in the cell (e.g., pH, reductive or oxidative environment, or reaction with intracellular species, such as glutathione). A bioreversible group incorporates within it a phosphate or phosphorothioate of a polynucleotide. Exemplary bioreversible groups include disulfides. Other exemplary bioreversible groups include thioesters,

The term “bulky group,” as used herein, represents any substituent or group of substituents as defined herein, in which the radical of the bulky group bears one hydrogen atom or fewer if the radical is sp³-hybridized carbon, bears no hydrogen atoms if the radical is sp²-hybridized carbon. The radical is not sp-hybridized carbon. The bulky group bonds to another group only through a carbon atom. For example, the statements “bulky group bonded to the disulfide linkage,” “bulky group attached to the disulfide linkage,” and “bulky group linked to the disulfide linkage” indicate that the bulky group is bonded to the disulfide linkage through a carbon radical.

The term “carbene” as used herein, represents a functional group that is a divalent carbon species having six valence electrons and the structure ═C: or —C(R^{B}): where R^{B} is selected from H, optionally substituted C₁₋₁₂ alkyl, optionally substituted C₆₋₁₄ aryl, optionally substituted (C₆₋₁₄ aryl)-C₁₋₋₁₂-alkylene, or optionally substituted carbonyl; and C is a carbon with two electrons that are not part of a covalent bond. The two electrons may be paired (e.g., singlet carbene) or unpaired (e.g., triplet carbene).

The term “carbocyclic,” as used herein, represents an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, and aryl groups.

The term “carbohydrate,” as used herein, represents a compound which comprises one or more monosaccharide units having at least 5 carbon atoms (which may be linear, branched or cyclic) with an oxygen, nitrogen or sulfur atom bonded to each carbon atom. The term “carbohydrate” therefore encompasses monosaccharides, disaccharides, trisaccharides, tetrasaccharides, oligosaccharides, and polysaccharides. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C₅₋₆ sugars; di- and trisaccharides include sugars having two or three monosaccharide units (e.g., C₅₋₆ sugars).

The term “carbonyl,” as used herein, represents a C(O) group. Examples of functional groups which comprise a “carbonyl” include esters, ketones, aldehydes, anhydrides, acyl chlorides, amides, carboxylic acids, and carboxlyates.

The term “complementary” in reference to a polynucleotide, as used herein, means Watson-Crick complementary.

The term “component of a coupling reaction,” as used herein, represents a molecular species capable of participating in a coupling reaction. Components of coupling reactions include hydridosilanes, alkenes, and alkynes.

The term “component of a cycloaddition reaction,” as used herein, represents a molecular species capable of participating in a cycloaddition reaction. In cycloaddition reactions in which bond formation involves [4n+2]-π electrons where n is 1, one component will provide 2-π electrons, and another component will provide 4-π electrons. Representative components of cycloaddition reactions that provide 2-π electrons include alkenes and alkynes. Representative components of cycloaddition reactions that provide 4-π electrons include 1,3-dienes, α, β-unsaturated carbonyls, and azides.

The term “conjugating group,” as used herein, represents a divalent or higher valency group containing one or more conjugating moieties. The conjugating group links one or more auxiliary moieties to a bioreversible group (e.g., a group containing a disulfide moiety).

The term “conjugating moiety,” as used herein, represents a functional group that is capable of forming one or more covalent bonds to another group (e.g., a functional group that is a nucleophile, electrophile, a component in a cycloaddition reaction, or a component in a coupling reaction) under appropriate conditions. The term also refers to the residue of a conjugation reaction, e.g., amide group. Examples of such groups are provided herein.

The term “coupling reaction,” as used herein, represents a reaction of two components in which one component includes a nonpolar a bond such as Si—H or C—H and the second component includes a Tr bond such as an alkene or an alkyne that results in either the net addition of the a bond across the Tr bond to form C—H, Si—C, or C—C bonds or the formation of a single covalent bond between the two components. One coupling reaction is the addition of Si—H across an alkene (also known as hydrosilylation). Other coupling reactions include Stille coupling, Suzuki coupling, Sonogashira coupling, Hiyama coupling, and the Heck reaction. Catalysts may be used to promote the coupling reaction. Typical catalysts are those which include Fe(II), Cu(I), Ni(0), Ni(II), Pd(0), Pd(II), Pd(IV), Pt(0), Pt(II), or Pt(IV).

The term “cycloaddition reaction” as used herein, represents reaction of two components in which [4n+2]π electrons are involved in bond formation when there is either no activation, activation by a chemical catalyst, or activation using thermal energy, and n is 1, 2, or 3. A cycloaddition reaction is also a reaction of two components in which [4n]-r electrons are involved, there is photochemical activation, and n is 1, 2, or 3. Desirably, [4n+2]-r electrons are involved in bond formation, and n=1. Representative cycloaddition reactions include the reaction of an alkene with a 1,3-diene (Diels-Alder reaction), the reaction of an alkene with an α,β-unsaturated carbonyl (hetero Diels-Alder reaction), and the reaction of an alkyne with an azido compound (Huisgen cycloaddition).

The term “cycloalkenyl,” as used herein, refers to a non-aromatic carbocyclic group having from three to ten carbons (e.g., a C₃-C₁₀ cycloalkylene), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenylene,” as used herein, refers to a divalent carbocyclic non-aromatic group having from three to ten carbons (e.g., C₃-C₁₀ cycloalkenylene), unless otherwise specified. Non-limiting examples of the cycloalkenylene include cycloprop-1-en-1,2-diyl; cycloprop-2-en-1,1-diyl; cycloprop-2-en-1,2-diyl; cyclobut-1-en-1,2-diyl; cyclobut-1-en-1,3-diyl; cyclobut-1-en-1,4-diyl; cyclobut-2-en-1,1-diyl; cyclobut-2-en-1,4-diyl; cyclopent-1-en-1,2-diyl; cyclopent-1-en-1,3-diyl; cyclopent-1-en-1,4-diyl; cyclopent-1-en-1,5-diyl; cyclopent-2-en-1,1-diyl; cyclopent-2-en-1,4-diyl; cyclopent-2-en-1,5-diyl; cyclopent-3-en-1,1-diyl; cyclopent-1,3-dien-1,2-diyl; cyclopent-1,3-dien-1,3-diyl; cyclopent-1,3-dien-1,4-diyl; cyclopent-1,3-dien-1,5-diyl; cyclopent-1,3-dien-5,5-diyl; norbornadien-1,2-diyl; norbornadien-1,3-diyl; norbornadien-1,4-diyl; norbornadien-1,7-diyl; norbornadien-2,3-diyl; norbornadien-2,5-diyl; norbornadien-2,6-diyl; norbornadien-2,7-diyl; and norbornadien-7,7-diyl. The cycloalkenylene may be unsubstituted or substituted (e.g., optionally substituted cycloalkenylene) as described for cycloalkyl.

The term “cycloalkyl,” as used herein, refers to a cyclic alkyl group having from three to ten carbons (e.g., a C₃-C₁0 cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted as defined herein (e.g., optionally substituted cycloalkyl). The cycloalkyl groups of this disclosure can be optionally substituted with: (1) alkanoyl (e.g., formyl, acetyl, and the like); (2) alkyl (e.g., alkoxyalkyl, alkylsulfinylalkyl, aminoalkyl, azidoalkyl, acylalkyl, haloalkyl (e.g., perfluoroalkyl), hydroxyalkyl, nitroalkyl, orthioalkoxyalkyl); (3) alkenyl; (4) alkynyl; (5) alkoxy (e.g., perfluoroalkoxy); (6) alkylsulfinyl; (7) aryl; (8) amino; (9) arylalkyl; (10) azido; (11) cycloalkyl; (12) cycloalkylalkyl; (13) cycloalkenyl; (14) cycloalkenylalkyl; (15) halo; (16) heterocyclyl (e.g., heteroaryl); (17) (heterocyclyl)oxy; (18) (heterocyclyl)aza; (19) hydroxy; (20) nitro; (21) thioalkoxy; (22) —(CH₂)CO₂R^{A}, where q is an integer from zero to four, and R^{A} is selected from the group consisting of (a)alkyl, (b)aryl, (c) hydrogen, and (d) arylalkyl; (23) —(CH₂)_{q}CONR^{B}R^{C}, where q is an integer from zero to four and where R^{B} and R^{C} are independently selected from the group consisting of (a) hydrogen, (b)alkyl, (c)aryl, and (d) arylalkyl; (24) —(CH₂)_{q}SO₂R^{D}, where q is an integer from zero to four and where R^{D} is selected from the group consisting of (a) alkyl, (b) aryl, and (c) arylalkyl; (25) —(CH₂)_{q}SO₂NR^{E}R^{F}, where q is an integer from zero to four and where each of R^{E} and R^{F} is, independently, selected from the group consisting of (a) hydrogen, (b) alkyl, (c) aryl, and (d) arylalkyl; (26) thiol; (27) aryloxy; (28) cycloalkoxy; (29) arylalkoxy; (30) heterocyclylalkyl (e.g., heteroarylalkyl); (31) silyl; (32) cyano; and (33) —S(O)R^{H} where R^{H} is selected from the group consisting of (a) hydrogen, (b) alkyl, (c) aryl, and (d) arylalkyl. In some embodiments, each of these groups can be further substituted as described herein.

The term “cycloalkyl alkyl,” as used herein, represents an alkyl group substituted with a cycloalkyl group. The cycloalkyl and alkyl portions may be substituted as the individual groups as described herein.

The term “electrophile” or “electrophilic group,” as used herein, represents a functional group that is attracted to electron rich centers and is capable of accepting pairs of electrons from one or more nucleophiles so as to form one or more covalent bonds. Electrophiles include, but are not limited to, cations; polarized neutral molecules; nitrenes; nitrene precursors such as azides; carbenes; carbene precursors; activated silicon centers; activated carbonyls; alkyl halides; alkyl pseudohalides; epoxides; electron-deficient aryls; activated phosphorus centers; and activated sulfur centers. Typically encountered electrophiles include cations such as H⁺ and NO⁺, polarized neutral molecules, such as HCl, alkyl halides, acyl halides, carbonyl containing compounds, such as aldehydes, and atoms which are connected to good leaving groups, such as mesylates, triflates, and tosylates.

The term “endosomal escape moiety,” as used herein, represents a moiety which enhances the release of endosomal contents or allows for the escape of a molecule from an internal cellular compartment such as an endosome.

The term “halo,” as used herein, represents a halogen selected from bromine, chlorine, iodine, and fluorine.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens, or, when the halogen group is F, haloalkyl group can be perfluoroalkyl. In some embodiments, the haloalkyl group can be further optionally substituted with 1, 2, 3, or 4 substituent groups as described herein for alkyl groups.

The term “heteroaryl,” as used herein, represents that subset of heterocyclyls, as defined herein, which are aromatic: i.e., they contain 4n+2 pi electrons within the mono- or multicyclic ring system. In one embodiment, the heteroaryl is substituted with 1, 2, 3, or 4 substituents groups as defined for a heterocyclyl group.

The term “heteroaryl alkyl,” as used herein, represents an alkyl group substituted with a heteroaryl group. The heteroaryl and alkyl portions may be substituted as the individual groups as described herein.

The term “heterocyclyl,” as used herein, represents a 5-, 6- or 7-membered ring, unless otherwise specified, containing one, two, three, or four heteroatoms independently selected from the group comprising nitrogen, oxygen, and sulfur. The 5-membered ring has zero to two double bonds, and the 6- and 7-membered rings have zero to three double bonds. Certain heterocyclyl groups include from 2 to 9 carbon atoms. Other such groups may include up to 12 carbon atoms. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, purinyl, thiadiazolyl (e.g., 1,3,4-thiadiazole), tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl and the like. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7H -purinyl); 1,2,3,6-tetrahydro-2,6-dioxo-1H -purinyl (e.g., 1,2,3,6-tetrahydro-3,7-dimethyl-2,6-dioxo-1H-purinyl); 2-oxobenz[c,d]indolyl; 1,1-dioxo-2H-naphth[1,8-c,d]isothiazolyl; and 1,8-naphthylenedicarboxamido. Heterocyclic groups also include groups of the formula

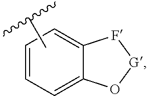

where

F′ is selected from the group consisting of —CH₂—, —CH₂O— and —O—, and G′ is selected from the group consisting of —C(O)— and —(C(R′)(R″))ᵥ—, where each of R′ and R″ is, independently, selected from the group consisting of hydrogen or alkyl of one to four carbon atoms, and v is one to three and includes groups, such as 1,3-benzodioxolyl, 1,4-benzodioxanyl, and the like. Any of the heterocyclyl groups mentioned herein may be optionally substituted with one, two, three, four or five substituents independently selected from the group consisting of: (1) alkanoyl (e.g., formyl, acetyl, and the like); (2) alkyl (e.g., alkoxyalkylene, alkylsulfinylalkylene, aminoalkylene, azidoalkylene, acylalkylene, haloalkylene (e.g., perfluoroalkyl), hydroxyalkylene, nitroalkylene, orthioalkoxyalkylene); (3) alkenyl; (4) alkynyl; (5) alkoxy (e.g., perfluoroalkoxy); (6) alkylsulfinyl; (7) aryl; (8) amino; (9) aryl-alkylene; (10) azido; (11) cycloalkyl; (12) cycloalkyl-alkylene; (13) cycloalkenyl; (14) cycloalkenyl-alkylene; (15) halo; (16) heterocyclyl (e.g., heteroaryl); (17) (heterocyclyl)oxy; (18) (heterocyclyl)aza; (19) hydroxy; (20) oxo; (21) nitro; (22) sulfide; (23) thioalkoxy; (24) —(CH₂)_{q}CO₂R^{A}, where q is an integer from zero to four, and R^{A} is selected from the group consisting of (a) alkyl, (b) aryl, (c) hydrogen, and (d) aryl-alkylene; (25) —(CH₂)CONR^{B}R^{C}, where q is an integer from zero to four and where R^{B} and R^{C} are independently selected from the group consisting of (a) hydrogen, (b) alkyl, (c) aryl, and (d) aryl-alkylene; (26) —(CH₂)SO₂R^{D}, where q is an integer from zero to four and where R^{D} is selected from the group consisting of (a) alkyl, (b) aryl, and (c) aryl-alkylene; (27) —(CH₂)_{q}SO₂NR^{E}R^{F}, where q is an integer from zero to four and where each of R^{E} and R^{F} is, independently, selected from the group consisting of (a) hydrogen, (b) alkyl, (c) aryl, and (d) aryl-alkylene; (28) thiol; (29) aryloxy; (30) cycloalkoxy; (31) arylalkoxy; (31) heterocyclyl-alkylene (e.g., heteroaryl-alkylene); (32) silyl; (33) cyano; and (34) —S(O)R^{H} where R^{H} is selected from the group consisting of (a) hydrogen, (b) alkyl, (c) aryl, and (d) aryl-alkylene. In some embodiments, each of these groups can be further substituted as described herein. For example, the alkylene group of an aryl-C₁-alkylene or a heterocyclyl-C₁-alkylene can be further substituted with an oxo group to afford the respective aryloyl and (heterocyclyl)oyl substituent group. In addition, when a heterocyclyl group is present in a bioreversible group of the invention it may be substituted with an ester, thioester, or disulfide group that is bound to a conjugating moiety, a hydrophilic functional group, or an auxiliary moiety as defined herein.

The term “heterocyclyl alkyl,” as used herein, represents an alkyl group substituted with a heterocyclyl group. The heterocyclyl and alkyl portions may be substituted as the individual groups as described herein.

The term “hydrophilic functional group,” as used herein, represents a moiety that confers an affinity to water and increases the solubility of an alkyl moiety in water. Hydrophilic functional groups can be ionic or non-ionic and include moieties that are positively charged, negatively charged, and/or can engage in hydrogen-bonding interactions. Exemplary hydrophilic functional groups include hydroxy, amino, carboxyl, carbonyl, thiol, phosphates (e.g., a mono-, di-, ortri-phosphate), polyalkylene oxides (e.g., polyethylene glycols), and heterocyclyls.

The terms “hydroxyl” and “hydroxy,” as used interchangeably herein, represent an —OH group.

The term “imine,” as used herein, represents a group having a double bond between carbon and nitrogen, which can be represented as “C═N.” In a particular embodiment, where a proton is a to the imine functional group, the imine may also be in the form of the tautomeric enamine. A type of imine bond is the hydrazone bond, where the nitrogen of the imine bond is covalently attached to a trivalent nitrogen (e.g., C═N—N(R)₂). In some embodiments, each R can be, independently, H, OH, optionally substituted C₁₋₆ alkoxy, or optionally substituted C₁₋₆ alkyl.

The term “internucleotide group,” as used herein, represents a group which covalently links two consecutive nucleosides together. The internucleotide group can be a non-bioreversible or a bioreversible group as defined herein. The internucleotide phosphorus (V) group is phosphate or phosphorothioate. One oxygen atom of the internucleotide group is at 3′ position of one nucleoside and another oxygen atom of the internucleotide group is at 5′ position of another adjacent nucleoside.

The term “loadable into a RISC complex,” as used herein, refers to the capability of a guide strand to be loaded into a RISC complex and the RISC-mediated degradation of a passenger strand hybridized to the guide strand. Thus, this polynucleotide does not include a non-bioreversible internucleotide group at 5′ position of a guide strand or the three contiguous nucleotides including a natural RISC-mediated cleavage site. The preferred natural RISC-mediated cleavage site is located on the passenger strand between two nucleosides that are complementary to the tenth and eleventh nucleotides of the guide strand.

The term “nitrene,” as used herein, represents a monovalent nitrogen species having six valence electrons and the structure ═N: or —NR^{A}: where R^{A} is selected from optionally substituted C₁₋₁₂ alkyl, optionally substituted C₆₋₁₂ aryl, optionally substituted (C₆₋₁₂ aryl)-C₁₋₁₂-alkylene, or optionally substituted carbonyl; and N is a nitrogen with four valence electrons, at least two of which are paired. The two remaining electrons may be paired (i.e., singlet nitrene) or unpaired (i.e., triplet nitrene).

The term “nitro,” as used herein, represents an —NO₂ group.

The term “non-bioreversible group,” as used herein, refers to a moiety including a functional group that is not a bioreversible group. The non-bioreversible group incorporates within it a phosphate or phosphorothioate of a polynucleotide. For example, the non-bioreversible group can be an internucleotide non-bioreversible group or a terminal non-bioreversible group, depending upon the point or points of attachment to the polynucleotide. An internucleotide non-bioreversible group contains a moiety including a functional group that is bonded to the oxygen or sulfur atom of the phosphate or phosphorothioate linking two nucleotides of a polynucleotide. A terminal non-bioreversible group contains a moiety including a functional group that is bonded to one or two oxygenand/or sulfur atoms of a terminal phosphate or the phosphorothioate of a polynucleotide. The non-bioreversible groups can include C₃₋₆ alkylene, alkenylene, alkynylene, arylene, arylalkylene, cycloalkylene, cycloalkyl alkylene, or cycloalkenylene bonded to the oxygen or sulfur atom of the phosphate or phosphorothioate, or any other linking group described herein.

A “non-naturally occurring amino acid” is an amino acid not naturally produced or found in a mammal.

By “nonpolar a bond” is meant a covalent bond between two elements having electronegativity values, as measured according to the Pauling scale, that differ by less than or equal to 1.0 units. Non-limiting examples of nonpolar a bonds include C—C, C—H, Si—H, Si—C, C—Cl, C—Br, C—I, C—B, and C—Sn bonds.

The term “nucleobase,” as used herein, represents a nitrogen-containing heterocyclic ring found at the 1′ position of the sugar moiety of a nucleotide or nucleoside. Nucleobases can be unmodified or modified. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; those disclosed in The Concise Encyclopedia Of Polymer Science And Engineering, pages 858-859, Kroschwitz, J. I., ed. John Wiley & Sons, 1990; those disclosed by Englisch et al., Angewandte Chemie, International Edition, 1991, 30, 613; and those disclosed by Sanghvi, Y. S., Chapter 15, Antisense Research and Applications, pages 289 302, (Crooke et al., ed., CRC Press, 1993). Certain nucleobases are particularly useful for increasing the binding affinity of the polymeric compounds of the invention, including 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications 1993, CRC Press, Boca Raton, pages 276-278). These may be combined, in particular embodiments, with 2′-O-methoxyethyl sugar modifications. United States patents that teach the preparation of certain of these modified nucleobases as well as other modified nucleobases include, but are not limited to, the above noted U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; and 5,681,941. For the purposes of this disclosure, “modified nucleobases,” as used herein, further represents nucleobases, natural or nonnatural, which comprise one or more protecting groups as described herein.

The terms “nucleophile,” as used herein, represent an optionally substituted functional group that engages in the formation of a covalent bond by donating electrons from electron pairs or -r bonds. Nucleophiles may be selected from alkenes, alkynes, aryl, heteroaryl, diaza groups, hydroxy groups, alkoxy groups, aryloxy groups, amino groups, alkylamino groups, anilido groups, thio groups, and thiophenoxy groups.

The term “nucleoside,” as used herein, represents a sugar-nucleobase combination. The sugar is a modified sugar containing a nucleobase at the anomeric carbon or a 3,5-dideoxypentafuranose containing a nucleobase at the anomeric carbon and a bond to another group at each position 3 and 5. The pentafuranose may be 3,5-dideoxyribose or 2,3,5-trideoxyribose or a 2 modified version thereof, in which position 2 is substituted with OR, R, halo (e.g., F), SH, SR, NH₂, NHR, NR₂, or CN, where R is an optionally substituted C₁₋₆ alkyl (e.g., (C₁₋₆ alkoxy)-C₁₋₆-alkyl) or optionally substituted (C₆₋₁₄ aryl)-C₁₋₄-alkyl. The modified sugars are non-ribose sugars, such as mannose, arabinose, glucopyranose, galactopyranose, 4-thioribose, and other sugars, heterocycles, or carbocycles. In some embodiments, the term “nucleoside” refers to a divalent group having the following structure:

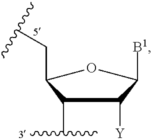

in which B¹ is a nucleobase; Y is H, halogen (e.g., F), hydroxyl, optionally substituted C₁₋₆ alkoxy (e.g., methoxy or methoxyethoxy), or a protected hydroxyl group; and each of 3′ and 5′ indicate the position of a bond to another group.

The term “nucleotide,” as used herein, refers to a nucleoside that further includes an internucleotide or a terminal phosphorus (V) group or a bioreversible or non-bioreversible group covalently linked to the 3′ or 5′ position of the divalent group. Nucleotides also include locked nucleic acids (LNA), glycerol nucleic acids, morpholino nucleic acids, and threose nucleic acids.

The terms “oxa” and “oxy,” as used interchangeably herein, represents a divalent oxygen atom that is connected to two groups (e.g., the structure of oxy may be shown as —O—).

The term “oxo,” as used herein, represents a divalent oxygen atom that is connected to one group (e.g., the structure of oxo may be shown as ═O).

The term “phosphorus (V) group,” as used herein, refers to a divalent group having the structure —O—P(═Z^{A})(—Z^{B})—O—, in which Z^{A} is O or S, and Z^{B} is OH, SH, or amino, or a salt thereof.

The term “polynucleotide” as used herein, represents a structure containing 11 or more contiguous nucleosides covalently bound together by any combination of internucleotide phosphorus (V), bioreversible, or non-bioreversible groups. Polynucleotides may be linear or circular.

The term “polypeptide,” as used herein, represents two or more amino acid residues linked by peptide bonds. Moreover, for purposes of this disclosure, the term “polypeptide” and the term “protein” are used interchangeably herein in all contexts. A variety of polypeptides may be used within the scope of the methods and compositions provided herein. In certain embodiments, polypeptides include antibodies or fragments of antibodies or antigen-binding fragments thereof. Polypeptides made synthetically may include substitutions of amino acids not naturally encoded by DNA (e.g., non-naturally occurring or unnatural amino acid).

The term “Ph,” as used herein, represents phenyl.

The terms “photolytic activation” or “photolysis,” as used herein, represent the promotion or initiation of a chemical reaction by irradiation of the reaction with light. The wavelengths of light suitable for photolytic activation range between 200-500 nm and include wavelengths that range from 200-260 nm and 300-460 nm. Other useful ranges include 200-230 nm, 200-250 nm, 200-275 nm, 200-300 nm, 200-330 nm, 200-350 nm, 200-375 nm, 200-400 nm, 200-430 nm, 200-450 nm, 200-475 nm, 300-330 nm, 300-350 nm, 300-375 nm, 300-400 nm, 300-430 nm, 300-450 nm, 300-475 nm, and 300-500 nm.

The term “protecting group,” as used herein, represents a group intended to protect a functional group (e.g., a hydroxyl, an amino, or a carbonyl) from participating in one or more undesirable reactions during chemical synthesis (e.g., polynucleotide synthesis). The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkylene ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkylene groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “sterically hindered,” as used herein, describes a chemical group having half-life of at least 24 hours in the presence of an intermolecular or an intramolecular nucleophile or electrophile.

The term “subject,” as used herein, represents a human or non-human animal (e.g., a mammal).

The term “sulfide” as used herein, represents a divalent —S— or ═S group.

The term “targeting moiety,” as used herein, represents any moiety that specifically binds or reactively associates or complexes with a receptor or other receptive moiety associated with a given target cell population.

The term “terminal group,” as used herein, refers to a group located at the first or last nucleoside in a polynucleotide. A 5′-terminal group is a terminal group bonded to 5′-carbon atom of the first nucleoside within a polynucleotide. A 3′-terminal group is a terminal group bonded to 3′-carbon atom of the last nucleoside within a polynucleotide.

The term “therapeutically effective dose,” as used herein, represents the quantity of an siRNA, or polynucleotide according to the invention necessary to ameliorate, treat, or at least partially arrest the symptoms of a disease or disorder (e.g., to inhibit cellular proliferation). Amounts effective for this use will, of course, depend on the severity of the disease and the weight and general state of the subject. Typically, dosages used in vitro may provide useful guidance in the amounts useful for in vivo administration of the pharmaceutical composition, and animal models may be used to determine effective dosages for treatment of particular disorders.

The term “thiocarbonyl,” as used herein, represents a C(═S) group. Non-limiting example of functional groups containing a “thiocarbonyl” includes thioesters, thioketones, thioaldehydes, thioanhydrides, thioacyl chlorides, thioamides, thiocarboxylic acids, and thiocarboxylates.

The term “thiol,” as used herein, represents an —SH group.

The term “disorder,” as used herein, is intended to be generally synonymous, and is used interchangeably with, the terms “disease,” “syndrome,” and “condition” (as in a medical condition), in that all reflect an abnormal condition presented by a subject, or one of its parts, that impairs normal functioning, and is typically manifested by distinguishing signs and symptoms.

The term “treating” as used in reference to a disorder in a subject, is intended to refer to reducing at least one symptom of the disorder by administrating a therapeutic (e.g., a nucleotide construct of the invention) to the subject.

As used herein and in the appended claims, the singular forms “a,” “and,” and “the” include plural referents unless the context clearly dictates otherwise. Thus, for example, reference to “a targeting moiety” includes a plurality of such targeting moieties, and reference to “the cell” includes reference to one or more cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

For purposes of this disclosure, any term present in the art which is identical to any term expressly defined in this disclosure, the term's definition presented in this disclosure will control in all respects.

### BRIEF DESCRIPTION OF THE DRAWINGS

FIG. 1A shows a siRNA of the invention containing two strands, where one of the strands contains disulfide linkages of the invention.

FIG. 1B shows a siRNA of the invention containing two strands, where both strands contain disulfide linkages of the invention.

FIG. 2 shows a representative polynucleotide construct of the invention and the RP-HPLC trace for the same polynucleotide.

FIG. 3 shows a mass spectrum of crude mixture of polynucleotide of the invention, the structure of which is shown in FIG. 2.

FIG. 4 shows a mass spectrum of purified polynucleotide of the invention, the structure of which is shown in FIG. 2.

FIG. 5A shows the structure of single-strand RNA constructs of the invention having one or three ADS conjugation sites.

FIG. 5B shows a photograph of the gel analysis of the single-strand RNA constructs of the invention. The structure of the constructs is described in FIGS. 6A, 6B, and 8.

FIG. 5C shows a photograph of the gel analysis of the single-strand RNA constructs of the invention. The structure of the constructs is described in FIGS. 6A, 6B, and 7A.

FIG. 5D shows a photograph of the gel analysis of the single-strand RNA constructs of the invention. The structure of the constructs is described in FIGS. 6A, 6B, and 7B.

FIG. 6A shows the general structure of representative siRNA constructs of the invention.

FIG. 6B shows the ADS conjugation group that is incorporated in the siRNA constructs shown in FIG. 6A.

FIG. 7A shows a structure of a representative targeting moiety (Folate) linked to a representative conjugating moiety.

FIG. 7B shows a structure of a representative targeting moiety (GaINAc) linked to a representative conjugating moiety.

FIG. 8 shows a structure of a representative targeting moiety (Mannose) linked to a representative conjugating moiety.

FIG. 9A is a chart showing certain exemplary bioreversible and non-bioreversible groups. FIG. 9B is a chart showing certain compounds used in the preparation of the polynucleotides listed in Table 7.

FIG. 10 shows two exemplary siRNA structures prior to [3+2] cycloaddition.

FIG. 11 shows a list of GaINAc-siRNA conjugates.

FIG. 12 shows the in vitro transfection data as determined according to the procedure described in Example 2. Strand 1 is a passenger strand, and strand 2 is a guide strand. Bars designated by each letter indicate IC₅₀ (pM) for one of the siRNA structures described in Table 9. SB-0165 is control. Each letter corresponds to the position of the internucleotide non-bioreversible group in the order from 5′ to 3′ (e.g., A of Strand 1 provides IC₅₀ data at 24 h and at 48 h for compound SB-0166, which includes a non-bioreversible connecting the first and the second nucleosides).

FIGS. 13A and 13B are graphs showing efficacy of exemplary siRNA compounds listed in Tables 5-7 in inhibiting ApoB gene expression in vitro in primary mouse hepatocytes from C57/Bl6 mouse. The determined IC₅₀ values are provided in tables under each graph.

FIG. 14A shows dose curves for siRNA conjugate of the invention ((Folate)₃-siRNN-Cy3) binding to KB cell.

FIG. 14B shows a graph determining dissociation constants (K_{d}) for siRNA conjugates of the invention ((Folate)₃-siRNN-Cy3 or (Folate)₁-siRNN-Cy3) and KB cells.

FIG. 15A shows dose curves for siRNA conjugate of the invention ((GaINAc)₉-siRNN-Cy3) binding to HepG2 cells.

FIG. 15B shows a graph determining dissociation constants (K_{d}) for siRNA conjugates of the invention ((GaINAc)₉-siRNN-Cy3 or (GaINAc)₃-siRNN-Cy3) and HepG2 cells.

FIG. 16A shows dose curves for siRNA conjugate of the invention (Mannose)₁₈-siRNN-Cy3 binding to primary peritoneal macrophages.

FIG. 16B shows a graph determining dissociation constants (K_{d}) for siRNA conjugates of the invention ((Mannose)₁₈-siRNN-Cy3 or (Mannose)₆-siRNN-Cy3) and primary peritoneal macrophages.

FIG. 17 is an image of NFKB-RE-Luc mice 4 hours after intraperitoneal administration of tumor necrosis factor-α (TNF-α). Comparison is provided to negative controls. The mice treated with siRNA of the invention exhibit diminished levels of Luciferase compared to the negative control mouse.

FIGS. 18A and 18B are graphs showing efficacy of an exemplary siRNA compound listed in Table 5 in inhibiting ApoB gene expression in vivo in C57Bl6 mice. FIG. 18A is a graph demonstrating dose response function at 72 hours measured by liver ApoB gene expression normalized to P2 microglobulin (B2M) gene expression in vivo versus administration of a vehicle only. FIG. 18B is a graph demonstrating time course of liver ApoB gene expression in vivo 96, 72, 48, and 24 hours following administration of siRNA (SB0097, see Table 5) normalized to B2M gene expression in vivo versus administration of vehicle only.

FIGS. 19A and 19B are graphs providing a comparison of the normalized ApoB expression levels for hybridized polynucleotide constructs of the invention relative to a vehicle.

FIG. 20A shows a structure of the positive control for the data in FIG. 20B. The positive control (SB-0165) includes 4 bioreversible groups (o-(t-butyldithio)phenethylphosphate) and one non-bioreversible group (homopropargyl phosphate connecting two nucleosides).

FIG. 20B shows the comparison for ApoB gene expression levels of the positive control shown in FIG. 20A and the same having a non-bioreversible triester E or Q, the letter designations being consistent with FIG. 12. Positive control with triester E is SB0190, and positive control with triester Q is SB0202.

FIGS. 21A and 21B are graphs showing GapDH expression normalized to the expression of a house-keeping gene. The GapDH expression was measured in macrophages isolated from mice that were administered intraperitoneally control (e.g., vehicle) or a hybridized polynucleotide construct of the invention.

FIG. 22 is a graph showing GapDH expression normalized to the expression of a house-keeping gene. The GapDH expression was measured in macrophages isolated from mice that were administered vehicle or a hybridized polynucleotide construct of the invention.

FIGS. 23A and 23B show results from mouse primary bone marrow cell experiments. FIG. 23A shows the normalized amount of mannose receptor expression in macrophages overtime. FIG. 23B shows a graph of GAPDH mRNA normalized to B2M after treatment with 48 hour treatment with exemplary siRNA compounds listed in Table 5. FIG. 23B shows the dose-dependent reduction in GapDH mRNA levels after administration of a hybridized polynucleotide construct of the invention.

FIGS. 24A and 24B are graphs showing dose-dependency of the GapDH expression and the related IC₅₀ data for the hybridized polynucleotides of the invention. The expression of GapDH was normalized to that of a house-keeping gene.

FIG. 25 is a photograph of a 15% denaturing gel stained with ethidium bromide showing bands of 2′-modified siRNA at the beginning (0 h) of incubation and after 24 h or 48 h at 37° C. in mouse serum. The three lanes on the right of the gel show bands obtained for hybridized polynucleotide constructs of the invention, and the three lanes on the left are control lanes (siRNA not having a phosphotriester group).

### DETAILED DESCRIPTION

The ability to deliver certain bioactive agents to the interior of cells is problematic due to the selective permeability of the cell plasma membrane. The plasma membrane of the cell forms a barrier that restricts the intracellular uptake of molecules to those which are sufficiently non-polar and smaller than approximately 500 daltons in size. Previous efforts to enhance the cellular internalization of proteins have focused on fusing proteins with receptor ligands (Ng et al., Proc. Natl. Acad. Sci. USA, 99:10706-11, 2002) or by packaging them into caged liposomal carriers (Abu-Amer et al., J. Biol. Chem. 276:30499-503, 2001). However, these techniques can result in poor cellular uptake and intracellular sequestration into the endocytic pathway. Due to their anionic charge and large size of about 14,000 Daltons, delivery of siRNA is a formidable challenge in mammals, including humans. However, cationically charged peptides and proteins have led to advancements in polynucleotide delivery. For example, linking peptide transduction domains (PTDs) to a nucleic acid has provided some advancement in polynucleotide delivery.

The invention provides hybridized polynucleotide constructs containing a passenger strand and a guide strand, where the passenger strand contains a 5′-terminal, a 3′-terminal, or an internucleotide non-bioreversible group, and/or the guide strand contains a 3′-terminal or an internucleotide non-bioreversible group. These hybridized polynucleotide constructs may exhibit a superior efficacy in gene silencing relative the hybridized polynucleotide constructs that differ only by the absence of the non-bioreversible group. Without being bound by theory, the superior efficacy may be due to an improvement in the kinetics of the RISC complex loading or an improvement in the stability of the hybridized polynucleotide construct.

The invention also provides nucleotide constructs comprising one or more bioreversible groups (e.g., disulfides). Sterically-hindered disulfides are particularly advantageous. Disulfides bonded to at least one bulky group exhibit greater stability during the nucleotide construct synthesis compared to disulfides that are not bonded to at least one bulky group, as the latter may react with a phosphorus (III) atom of the nucleotide construct to cleave the disulfide bond.

Relatively large moieties, e.g., a polypeptide, a carbohydrate, a neutral organic polymer, a positively charged polymer, a therapeutic agent, a targeting moiety, an endosomal escape moiety, or combination thereof, may be included in bioreversible groups, without affecting the ability of the bioreversible group to be cleaved intracellularly. The invention also provides for nucleotide constructs comprising bioreversible groups that have hydrophobic or hydrophilic functional groups, and/or conjugating moieties, where these conjugating moieties allow for attachment of a polypeptide, a small molecule, a carbohydrate, a neutral organic polymer, a positively charged polymer, a therapeutic agent, a targeting moiety, an endosomal escape moiety, or any combination thereof to an internucleotide or a terminal phosphate or phosphorothioate. The invention further provides for a nucleotide construct that comprises one or more bioreversible groups comprising one or more hydrophobic or hydrophilic functional groups, and/or one or more conjugating groups having one or more conjugating moieties that allow for the attachment of an auxiliary moiety, e.g., a polypeptide, a small molecule, a carbohydrate, a neutral organic polymer, a positively charged polymer, a therapeutic agent, a targeting moiety, an endosomal escape moiety, or any combination thereof, to the nucleotide construct. In one embodiment, the nucleotide constructs disclosed herein contain a certain number of bioreversible groups reducing the overall negative charge of the constructs, thereby allowing for or facilitating the uptake of the constructs by a cell. The nucleotide constructs described herein can allow for or facilitate the intracellular transport of a polynucleotide itself or a polynucleotide linked to an attached auxiliary moiety, e.g., a small molecule, peptide, polypeptide, carbohydrate, neutral organic polymer, positively charged polymer, therapeutic agent, targeting moiety, endosomal escape moiety, or combination thereof. The action of intracellular enzymes (e.g., intracellular protein disulfide isomerase, thioredoxin, or thioesterases) or exposure to the intracellular environment can result in the cleavage of the disulfide or thioester linkage, thereby releasing the auxiliary moiety and/or unmasking the polynucleotide. The unmasked polynucleotide can then, e.g., initiate an antisense or RNAi-mediated response. Further, the nucleotide constructs of the invention also allow for or facilitate the intracellular delivery of a polynucleotide or a polynucleotide linked through a disulfide or a thioester linkage to an attached auxiliary moiety, e.g., a small molecule, peptide, polypeptide, carbohydrate, neutral organic polymer, positively charged polymer, therapeutic agent, targeting moiety, endosomal escape moiety, or combination thereof, without the need for carriers, such as liposomes, or cationic lipids. Preferably, the linkage between the auxiliary moiety and the polynucleotide includes a disulfide linkage. Each of the features is further described herein.

The invention provides methods and compositions to facilitate and improve the cellular uptake of polynucleotides by reducing or neutralizing the charge associated with anionically charged polynucleotides, and optionally adding further functionality to the molecule, e.g., cationic peptides, targeting moiety, and/or endosomal escape moiety. In particular embodiments, the compositions of the invention may promote uptake of a polynucleotide by generating nucleotide constructs that have a cationic charge.

The invention provides compositions and methods for the delivery of sequence specific polynucleotides useful for selectively treating human disorders and for promoting research. The compositions and methods of the invention effectively deliver polynucleotides, including siRNAs, RNA, and DNA to subjects and to cells, without the drawbacks of current nucleic acid delivery methods. The invention provides compositions and methods which overcome size and charge limitations that make RNAi constructs difficult to deliver into cells or make the constructs undeliverable. By reversibly neutralizing the anionic charge of nucleic acids (e.g., dsRNA), a nucleotide construct comprising a bioreversible group according to the invention can deliver nucleic acids into a cell in vitro and in vivo.

The invention provides nucleotide constructs comprising a charge neutralizing moiety (e.g., non-bioreversible group, a bioreversible group; or a component (i), a group of formula (II), or a group of formula (IIa) used as a protecting group for an internucleotide or a terminal phosphorus (V) group). The construct can further include auxiliary moieties useful in cellular transfection and cellular modulation. Such auxiliary moieties can include a small molecule, peptide, a polypeptide, a carbohydrate, a neutral organic polymer, a positively charged polymer, a therapeutic agent, a targeting moiety, an endosomal escape moiety, or any combination thereof.

The invention provides compositions and methods for the delivery of nucleotide constructs comprising one or more targeting moieties for targeted delivery to specific cells (e.g., cells having asialoglycoprotein receptors on their surface (e.g., hepatocytes), tumor cells (e.g., tumor cells having folate receptors on their surface), cells bearing mannose receptor (e.g., macrophages, dendritic cells, and skin cells (e.g., fibroblasts or keratinocytes))). Non-limiting examples of mannose receptor superfamily include MR, Endo180, PLA2R, MGL, and DEC205. Targeted delivery of the nucleotide constructs of the invention may involve receptor mediated internalization. In some embodiments, targeting moieties may include mannose, N-acetyl galactosamine (GaINAc), or a folate ligand.

As demonstrated herein, the addition of one or more removable (e.g., reversibly attached) charge neutralizing moieties to a nucleic acid can facilitate cell transfection. Any nucleic acid, regardless of sequence composition, can be modified. Accordingly, the invention is not limited to any particular sequence (i.e., any particular siRNA, dsRNA, DNA or the like).

The invention provides nucleotide constructs having, in some embodiments, one or more bioreversible moieties that contribute to chemical and biophysical properties that enhance cellular membrane penetration and resistance to exo- and endonuclease degradation. The invention further provides reagents for the synthesis of the nucleotide constructs disclosed herein, e.g., phosphoramidite reagents. Moreover, these bioreversible groups are stable during the synthetic processes.

In cells, the bioreversible moieties can be removed by the action of enzymes (e.g., enzymes having thioreductase activity (e.g., protein disulfide isomerase orthioredoxin)) or by exposure to the intracellular conditions (e.g., an oxidizing or reducing environment) or reactants (e.g., glutathione or other free thiol) to yield biologically active polynucleotide compounds that are capable of hybridizing to and/or having an affinity for specific endogenous nucleic acids.

The bioreversible moieties can be used with antisense polynucleotides of synthetic DNA or RNA or mixed molecules of complementary sequences to a target sequence belonging to a gene or to an mRNA whose expression they are specifically designed to block or down-regulate. These inhibitory polynucleotides may be directed against a target mRNA sequence or, alternatively against a target DNA sequence, and hybridize to the nucleic acid to which they are complementary thereby inhibiting transcription or translation. Accordingly, the nucleotide constructs disclosed herein can effectively block or down-regulate gene expression.

The nucleotide constructs of the invention may also be directed against certain bicatenary DNA regions (homopurine/homopyrimidine sequences or sequences rich in purines/pyrimidines) and thus form triple helices. The formation of a triple helix, at a particular sequence, can block the interaction of protein factors which regulate or otherwise control gene expression and/or may facilitate irreversible damage to be introduced to a specific nucleic acid site if the resulting polynucleotide is made to possess a reactive functional group.

Polynucleotides

The invention provides nucleotide constructs that contain polynucleotides (“polynucleotide constructs”) having one or more charge neutralizing groups (e.g., a bioreversible group, a non-bioreversible group; or a component (i), a group of formula (II), or a group of formula (IIa)) attached to an internucleotide or terminal phosphorus (V) group). The one or more charge neutralizing groups can contain a bioreversible group, such as a disulfide or a thioester linkage. Preferably, the one or more charge neutralizing groups include a disulfide linkage. The one or more charge neutralizing groups can contain one or more auxiliary moieties linked to the internucleotide phosphorus (V) group or terminal phosphorus (V) group (e.g., a bioreversible group having a disulfide or a thioester linkage; preferably, a disulfide linkage). Examples of such auxiliary moieties include a small molecule, a conjugating moiety, a hydrophilic functional group, a polypeptide, a carbohydrate, a neutral organic polymer, a positively charged polymer, a therapeutic agent, a targeting moiety, an endosomal escape moiety, and any combination thereof. The bioreversible group may be able to undergo a separate reaction, e.g., intramolecularly, to leave an unmodified internucleotide bridging group or terminal nucleotide group. While various sugars and backbones can be employed, as described in the definition of nucleotide provided herein, the polynucleotide will typically employ a ribose, deoxyribose, or LNA sugar and phosphate orthiophosphate internucleotide phosphorus (V) groups. Mixtures of these sugars and bridging groups in a single polynucleotide are also contemplated.

The polynucleotides constructs described herein feature bioreversible groups that can be selectively cleaved intracellularly (e.g., by exposure to the passive environment, action of enzymes, or other reactants) thereby facilitating the intracellular delivery of polynucleotides to cells. Exemplary bioreversible groups include disulfide linkages.

For example, the polynucleotide constructs described herein can include disulfide linkages that can be cleaved by intracellular enzymes having thioreductase activity. Upon entry into a cell, these disulfide linkages (e.g., those contained between A¹ group and A² group of formula (II)) can be selectively cleaved by enzymes in order to unmask the nucleic acid. Disulfide linkages described herein can also provide a useful handle by which to functionalize the nucleic acid with one or more auxiliary moieties (e.g., one or more targeting moieties) and other conjugates, or with groups that will modify the physicochemical properties of the nucleic acid (e.g., hydrophilic groups such as hydroxy (—OH) groups). The strategy can be readily generalized to a number of structurally and functionally diverse nucleic acids in order to allow for targeted cellular delivery without the use of separate delivery agents.

The polynucleotide constructs described herein can include, e.g., 1-40 independent bioreversible groups or non-bioreversible group. For example, the polynucleotide constructs disclosed herein can include between 1-30, 1-25, 1-20, 2-15, 2-10, or 1-5 independent bioreversible or non-bioreversible groups. In particular embodiments, no more than 75% of the constituent nucleotides include a bioreversible group (e.g., no more than 20%, 30%, 40%, 50%, 55%, 60%, 65%, 70%, or 75% include a bioreversible group). In another embodiment, up to 90% of nucleotides within a polynucleotide construct of the invention can have a bioreversible group. In yet another embodiment, no more than half of the bioreversible groups will include hydrophobic termini, e.g., alkyl groups (e.g., when (R⁴)r-L-A¹ combine to form a hydrophobic group). In certain embodiments, no more than 75% of the constituent nucleotides include a non-bioreversible group (e.g., no more than 20%, 30%, 40%, 50%, 55%, 60%, 65%, 70%, or 75% include a bioreversible group). The polynucleotide constructs disclosed herein can feature any combination of bioreversible groups, e.g., that include a conjugating moiety, a hydrophilic functional group, a polypeptide, a small molecule, a carbohydrate, a neutral organic polymer, a positively charged polymer, a therapeutic agent, a targeting moiety, an endosomal escape moiety, or any combination thereof. The polynucleotide construct will generally be up to 150 nucleotides in length. In some embodiments, the polynucleotide construct consists of 5-100, 5-75, 5-50, 5-25, 8-40, 10-32, 15-30, or 19-28 nucleotides in length.

In certain embodiments, the polynucleotide construct contains one or more components (i) or groups of formula (II) each of the components contains, independently, a polypeptide, a carbohydrate, a neutral organic polymer, a positively charged polymer, a therapeutic agent, a targeting moiety, or an endosomal escape moiety; where each of the components (i) and groups of formula (II) includes a linker to an internucleotide bridging group of the polynucleotide construct, the linker containing a disulfide or a thioester (preferably, a disulfide, e.g., the linker is -L-A¹-S-S-A²-A³-A⁴-) and one or more bulky groups proximal to the disulfide group and rendering the disulfide group sterically hindered.

In some embodiments, the polynucleotide construct contains one or more components (i) each of the components contains, independently, a polypeptide, a carbohydrate, a neutral organic polymer, a positively charged polymer, a therapeutic agent, a targeting moiety, or an endosomal escape moiety In particular embodiments, the locations of bioreversible groups within a polynucleotide construct are selected so as to improve the stability of the resulting construct (e.g., to increase half life of the polynucleotide construct in the absence of the reagents (e.g., an oxidizing or reducing environment) responsible for cleaving the disulfide linkage). In particular, for double stranded polynucleotides, the location of the bioreversible groups will be such that a stable at mammalian physiological temperature double-stranded molecule is formed.

In other embodiments, the nature of each bioreversible group can be selected so as to generate favorable solubility and delivery properties. Such variations can include modulating the linker length, e.g., between the internucleotide bridging group or terminal nucleotide group and the disulfide group and/or between the disulfide group and any conjugating moiety, hydrophilic functional group, or auxiliary moiety. Reductions in solubility caused by hydrophobic bioreversible groups can be offset, in part, by the use of one or more hydrophilic bioreversible groups elsewhere in the polynucleotide. In a particular embodiment, the nucleoside bonded to a bioreversible group does not include a 2′ OH group, e.g., includes a 2′ F or OMe group instead.

For example, some of the polynucleotide constructs described herein can include a structure according to Formula I,

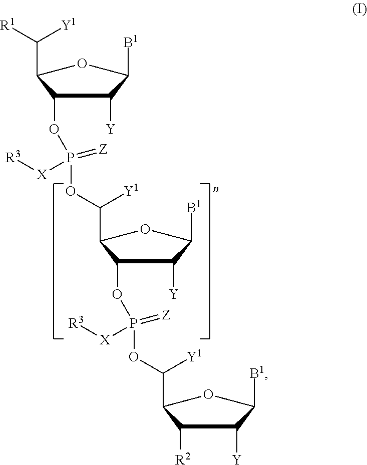

or a salt thereof,

where n is a number from 0 to 150;

each B¹ is independently a nucleobase;

each X is independently selected from the group consisting of absent, O, S, and optionally substituted N;

each Y is independently selected from the group consisting of hydrogen, hydroxyl, halo, optionally substituted C₁₋₆ alkoxy, and a protected hydroxyl group;

each Y¹ is independently H or optionally substituted C₁₋₆ alkyl (e.g., methyl);

each Z is independently O or S;

R¹ is selected from the group consisting of H, hydroxyl, optionally substituted C₁₋₆ alkoxy, a protected hydroxyl group, a monophosphate, a diphosphate, a triphosphate, a tetraphosphate, a pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, and a bond to a linker connecting to an oligonucleotide, and any combination thereof, or R¹ is

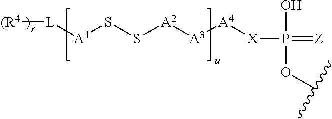

or a salt thereof;

R² is selected from the group consisting of H, hydroxyl, optionally substituted C₁₋₆ alkoxy, a protected hydroxyl group, a monophosphate, a diphosphate, a triphosphate, a tetraphosphate, a pentaphosphate, an optionally substituted C₁₋₆ alkyl, an amino containing group, a biotin containing group, a digoxigenin containing group, a cholesterol containing group, a quencher containing group, a phosphothiol, a polypeptide, a carbohydrate, a neutral organic polymer, a positively charged polymer, a therapeutic agent, a targeting moiety, an endosomal escape moiety, and any combination thereof, or R² is

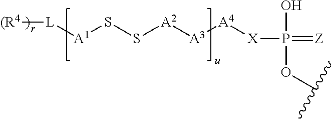

or a salt thereof; and

each R³ is independently absent, a hydrogen, optionally substituted C₁₋₆ alkyl, or a group having the structure of Formula II:

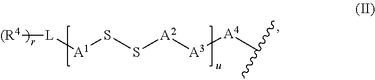

where each A¹ is independently a bond or a linker containing or being one or more of optionally substituted N; O; S; optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁-4-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A¹ includes one or more of optionally substituted N, O, and S, the optionally substituted N, O, or S is not directly bonded to the disulfide; and each A² is independently selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with -S-S-, join to form an optionally substituted 5 to 16 membered ring;

each A³ is independently selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;

each A⁴ is independently selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;

each L is independently absent or a conjugating group including or consisting of one or more conjugating moieties;

each R⁴ is independently hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;

each r is independently an integer from 1 to 10;

each u is independently 0 or 1;

where, in at least one of R¹, R², and R³, A², A³, and A⁴ combine to form a group having at least three atoms in the shortest chain connecting -S-S- and X; and

where at least one R³ has the structure of formula (II).

In some embodiments, L includes a bond to another polynucleotide (e.g., another polynucleotide of formula (I)). In particular embodiments, Y¹ is H.

The disulfide linkage in the polynucleotide and nucleotides of the invention may be replaced by another bioreversible group, e.g., a thioester moiety. For example, the group of formula (II), (IIa), (VIII), or (VIIIa) may be replaced with the group of formula (IIb):

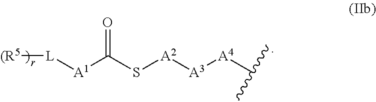

The synthetic methods described herein can be adapted to prepare such polynucleotides and nucleotides. Thus, the thioester-containing groups are considered to be within the scope of the present invention.

Certain embodiments of formula (I) include those in which X and Z are both O (e.g., a phosphate). In some embodiments, polynucleotide constructs disclosed herein largely comprise the structure of formula (I) but the depicted internucleotide phosphorus (V) group of formula ( ) is replaced with another internucleotide phosphorus (V) group (e.g., modified polynucleotide backbones) described herein. In alternate embodiments, polynucleotide constructs disclosed herein largely contain the structure of formula (I) but the depicted group R¹ and/or R² of formula (I) is replaced with a terminal nucleotide group having group R³. Polynucleotide constructs disclosed herein may have modified polynucleotide backbones. Examples of modified polynucleotide backbones include, for example, phosphorothioates, chiral phosphorothioates, phosphorodithioates, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, where the adjacent pairs of nucleoside units are linked 3′-5′ to 5′-3′ or 2′-5′ to 5′-2′. Representative United States patents that teach the preparation of the above phosphorus-containing linkages include U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference. Nucleotide constructs disclosed herein having modified polynucleotide backbones that do not include a phosphorus atom therein may have backbones that are formed by short chain alkyl or cycloalkyl internucleotide bridging groups, mixed heteroatom and alkyl or cycloalkyl internucleotide bridging groups, or one or more short chain heteroatomic or heterocyclic internucleotide bridging groups. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above polynucleotides include U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Exemplary -A¹-S-S-A²-A³-A⁴- or -S-S-A²-A³-A⁴- groups are as follows:

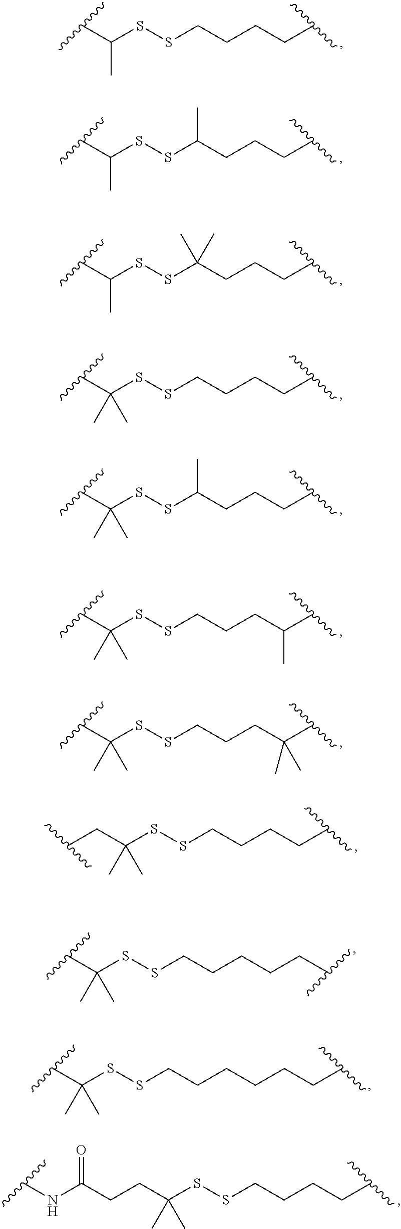

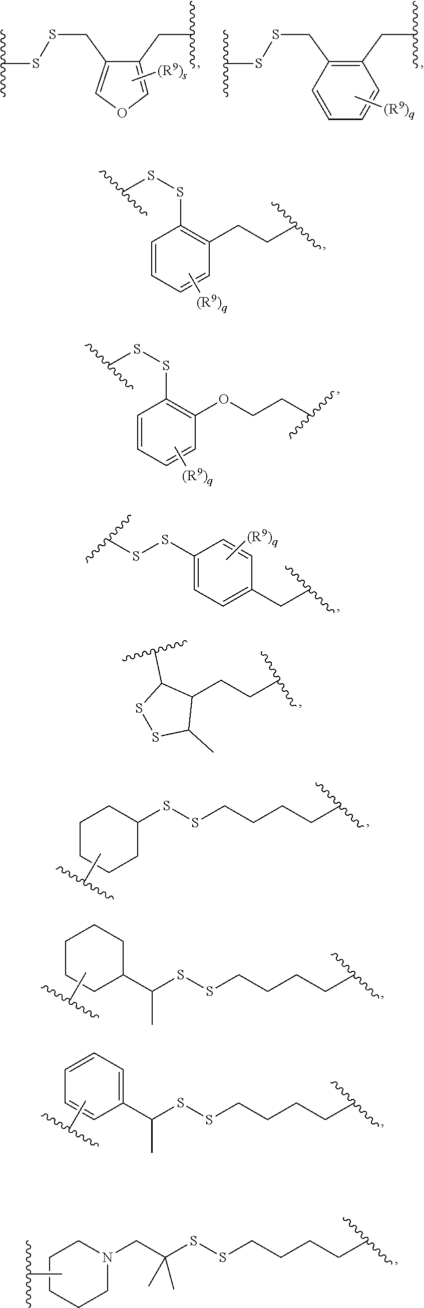

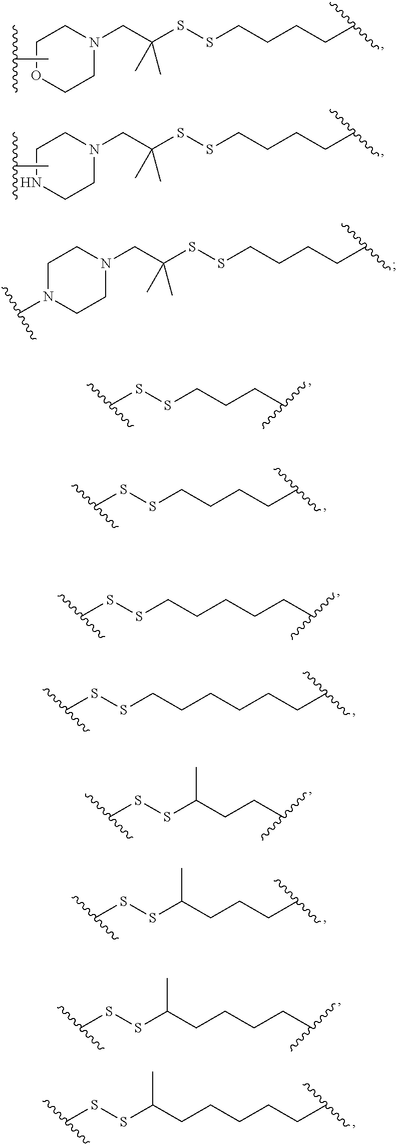

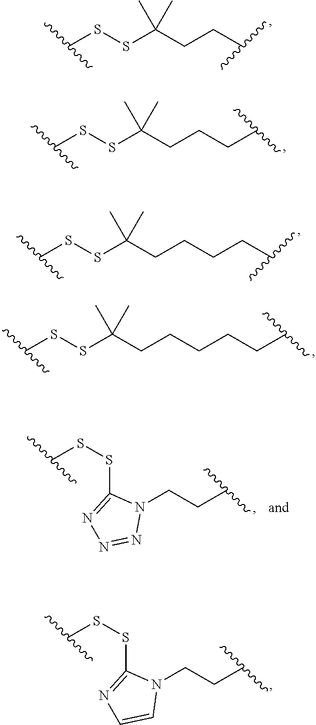

where

each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁-4-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from nitrogen, oxygen, and sulfur; optionally substituted (C₁₋₉ heteroaryl)-C₁₄-alkyl having 1 to 4 heteroatoms selected from nitrogen, oxygen; optionally substituted C₁₋₉ heterocyclyl having 1 to 4 heteroatoms selected from nitrogen, oxygen, and sulfur; optionally substituted (C₁₋₉ heterocyclyl)-C₁₋₄-alkyl having 1 to 4 heteroatoms selected from nitrogen, oxygen, and sulfur; amino; or optionally substituted C₁₋₆ alkoxy; or two adjacent R⁹ groups, together with the atoms to which each the R⁹ is attached, combine to form a cyclic group selected from the group consisting of C₆ aryl, C₂₋₅ heterocyclyl, or C₂₋₅ heteroaryl, where the cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4; and

s is 0, 1, or 2.

Exemplary groups included in the bioreversible groups of the invention are the following:

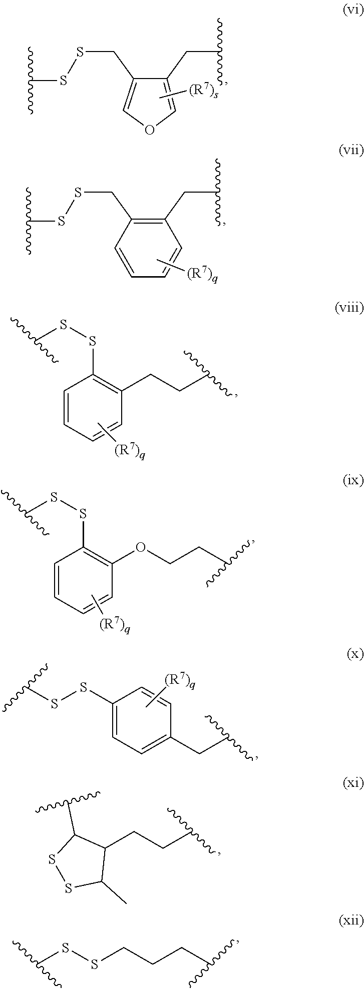

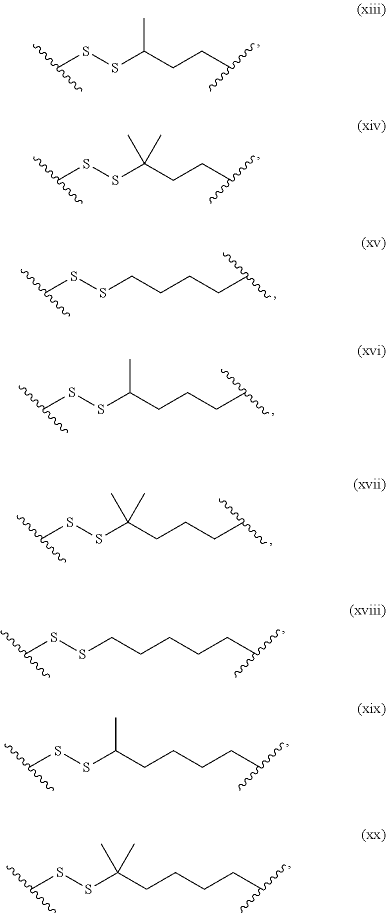

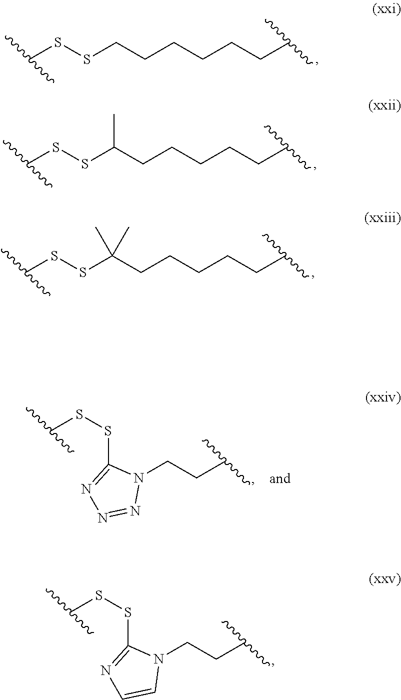

where

each R⁷ is independently C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁ aryl)-C₁₋₄-alkyl; —(CH₂)CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)SO₂NR^{E}R^{F}, where an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁₋₆ alkyl, C₆₋₁₀ aryl, and (C₆₋₁₀ aryl)-C₁₋₄-alkyl; or two adjacent R⁷ groups, together with the atoms to which each the R⁷ is attached combine to form a cyclic group selected from the group consisting of C aryl, C₂₋₅ heterocyclyl, or C₂-heteroaryl, where the cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁ aryl)-C₁₋₄-alkyl; —(CH₂)_{q}CONR^{B}R^{C}, where is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)SO₂NR^{E}R^{F}, where is an integer from zero to four and where each f R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₄-alkyl;

q is 0, 1, 2, 3, or 4; and

s is 0, 1, or 2.

The invention further provides methods for manufacturing the polynucleotide constructs of the invention. Methods for the preparation of nucleotides and polynucleotides are known in the art. For example, the practice of phosphoramidite chemistry to prepare polynucleotides is known from the published work of Caruthers and Beaucage and others. See, e.g., U.S. Pat. Nos. 4,458,066; 4,500,707; 5,132,418; 4,415,732; 4,668,777; 4,973,679; 5,278,302, 5,153,319; 5,218,103; 5,268,464; 5,000,307; 5,319,079; 4,659,774; 4,672,110; 4,517,338; 4,725,677; and RE34,069, each of which is herein incorporated by reference, describe methods of polynucleotide synthesis. Additionally, the practice of phosphoramidite chemistry has been systematically reviewed by Beaucage et al., Tetrahedron, 48: 2223-2311, 1992; and Beaucage et al., Tetrahedron, 49:6123-6194, 1993, as well as references referred to therein, all of which are herein incorporated by reference.

Nucleic acid synthesizers are commercially available, and their use is generally understood by persons of ordinary skill in the art as being effective in generating nearly any polynucleotide of reasonable length which may be desired.

In practicing phosphoramidite chemistry, useful 5′OH sugar blocking groups are trityl, monomethoxytrityl, dimethoxytrityl and trimethoxytrityl, especially dimethoxytrityl (DMTr). In practicing phosphoramidite chemistry, useful phosphite activating groups are dialkyl substituted nitrogen groups and nitrogen heterocycles. One approach includes the use of the di-isopropylamino activating group.

Polynucleotides can be synthesized by a Mermade-6 solid phase automated polynucleotide synthesizer or any commonly available automated polynucleotide synthesizer. Triester, phosphoramidite, or hydrogen phosphonate coupling chemistries (described in, for example, M. Caruthers, Oligonucleotides: Antisense Inhibitors of Gene Expression, pp. 7-24, J. S. Cohen, ed. (CRC Press, Inc. Boca Raton, Fla., 1989); Oligonucleotide synthesis, a practical approach, Ed. M. J. Gait, IRL Press, 1984; and Oligonucleotides and Analogues, A Practical Approach, Ed. F. Eckstein, IRL Press, 1991) are employed by these synthesizers to provide the desired polynucleotides. The Beaucage reagent, as described in, for example, Journal of American Chemical Society, 112:1253-1255, 1990, or elemental sulfur, as described in Beaucage et al., Tetrahedron Letters 22:1859-1862, 1981, is used with phosphoramidite or hydrogen phosphonate chemistries to provide substituted phosphorothioate polynucleotides.

For example, the reagents containing the protecting groups recited herein can be used in numerous applications where protection is desired. Such applications include, but are not limited to, both solid phase and solution phase, polynucleotide synthesis and the like.

For instance, structural groups are optionally added to the ribose or base of a nucleoside for incorporation into a polynucleotide, such as a methyl, propyl or allyl group at the 2′-O position on the ribose, or a fluoro group which substitutes for the 2′-O group, or a bromo group on the ribonucleoside base. For use with phosphoramidite chemistry, various phosphoramidite reagents are commercially available, including 2′-deoxy phosphoramidites, 2′-O-methyl phosphoramidites and 2′-O-hydroxyl phosphoramidites. Any other means for such synthesis may also be employed. The actual synthesis of the polynucleotides is well within the talents of those skilled in the art. It is also well known to use similar techniques to prepare other polynucleotides such as the phosphorothioates, methyl phosphonates and alkylated derivatives. It is also well known to use similar techniques and commercially available modified phosphoramidites and controlled-pore glass (CPG) products such as biotin, Cy3, fluorescein, acridine or psoralen-modified phosphoramidites and/or CPG (available from Glen Research, Sterling Va.) to synthesize fluorescently labeled, biotinylated or other conjugated polynucleotides.

Formula (Ia):

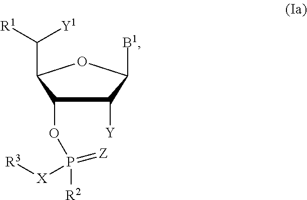

or a salt thereof,

B¹ is a nucleobase;

X is O, S, or optionally substituted N;

Y is a hydrogen, hydroxyl, halo, optionally substituted C₁₋₆ alkoxy, or a protected hydroxyl group;

Y¹ is independently H or optionally substituted C₁₋₆ alkyl (e.g., methyl);

Z is absent;

R¹ is protected hydroxyl (e.g., 4,4′-dimethoxytrityl group (DMT));

R² is —N(R³)R⁴ or —N(C₁₋₆ alkyl)₂ (e.g., —N(iPr)₂); and

R³ is a group having the structure of Formula (IIa):

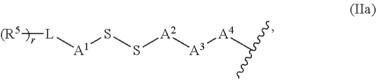

where A¹ is a bond or a linker containing or consisting of one or more of optionally substituted N, O, S, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from nitrogen, oxygen, and sulfur; optionally substituted (C₁₋₉ heteroaryl)-C₁-4-alkylene having 1 to 4 heteroatoms selected from nitrogen, oxygen; optionally substituted C₁₋₉ heterocyclylene having 1 to 4 heteroatoms selected from nitrogen, oxygen, and sulfur; and optionally substituted (C₁₋₉ heterocyclyl)-C₁₋₄-alkylene having 1 to 4 heteroatoms selected from nitrogen, oxygen, and sulfur, provided that when A¹ comprises one or more of amino, O, and S, none of the amino, O, and S is directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from nitrogen, oxygen, and sulfur; and optionally substituted C₁₋₉ heterocyclylene having 1 to 4 heteroatoms selected from nitrogen, oxygen, and sulfur; or A¹ and A², together with -S-S-, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from nitrogen, oxygen, and sulfur; optionally substituted C₁₋₉ heterocyclylene having 1 to 4 heteroatoms selected from nitrogen, oxygen, and sulfur; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from nitrogen, oxygen, and sulfur;

L is a bond or a conjugating group including or consisting of one or more conjugating moieties;

R⁵ is hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;

r is an integer from 1 to 10;

where A², A³, and A⁴ combine to form a group having at least three atoms in the shortest chain connecting -S-S- and X; and

each R⁴ and R⁶ is independently selected from the group consisting of hydrogen; optionally substituted C₁₋₆ alkyl; optionally substituted C₂₋₇ alkanoyl; hydroxyl; optionally substituted C₁₋₆ alkoxy; optionally substituted C₃₋₈ cycloalkyl; optionally substituted C₃₋₈ cycloalkenyl; optionally substituted C₆₋₁₄ aryl; optionally substituted C₆₋₁₅ aryloyl; optionally substituted C₁₋₉ heterocyclyl having 1 to 4 heteroatoms selected from nitrogen, oxygen, and sulfur; and optionally substituted C₃-1₀ (heterocycle)oyl having 1 to 4 heteroatoms selected from nitrogen, oxygen, and sulfur.

The invention further provides methods to process a polynucleotide construct synthesized by using a method of manufacture disclosed herein. For example, post synthesis of the polynucleotide construct, if a nucleobase contains one or more protecting groups, the protecting groups may be removed; and/or for any -L-A¹-S-S-A²-A³-A⁴- containing a hydrophilic functional group or conjugating moiety that is protected by a protecting group, then the protecting group may be removed.

Additionally, post synthesis of the polynucleotide construct, a group containing one or more of a small molecule, a polypeptide, a carbohydrate, a neutral organic polymer, a positively charged polymer, a therapeutic agent, a targeting moiety, and an endosomal escape moiety can be linked to one or more conjugating moieties of one or more bioreversible groups.

Nucleotides

The invention may employ compounds containing a single nucleotide (“compound of the invention”). Such a compound may have a structure according to Formula (VII):

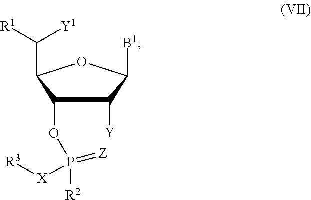

or a salt thereof,

where

B¹ is a nucleobase;

X is O, S, or NR⁴;

Y is hydrogen, hydroxyl, halo, optionally substituted C₁₋₆ alkoxy, or a protected hydroxyl group;

Y¹ is independently H or optionally substituted C₁₋₆ alkyl (e.g., methyl);

Z is absent, O, or S;

R¹ is hydroxyl, optionally substituted C₁₋₆ alkoxy, a protected hydroxyl group, a monophosphate, a diphosphate, a triphosphate, a tetraphosphate, and a pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, or any combination thereof;

R² is H, hydroxyl, optionally substituted C₁₋₆ alkoxy, a protected hydroxyl group, a monophosphate, a diphosphate, a triphosphate, a tetraphosphate, a pentaphosphate, and an amino, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, or any combination thereof; and

R³ is a group having the structure of Formula (VIII):

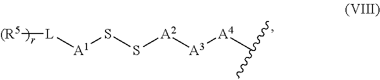

where

A¹ is a bond or a linker including or consisting of one or more of optionally substituted N; O; S; optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from N, O, and S; optionally substituted (C₁₋₉ heteroaryl)-C₁₋₄-alkylene having 1 to 4 heteroatoms selected from N, O, and S; optionally substituted C₁₋₉ heterocyclylene having 1 to 4 heteroatoms selected from N, O, and S; and optionally substituted (C₁₋₉ heterocyclyl)-C₁₋₄-alkylene having 1 to 4 heteroatoms selected from N, O, and S, provided that when A¹ comprises one or more of optionally substituted N, O, and S, the optionally substituted N, O, or S is not directly bonded to the disulfide; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; and optionally substituted C₁₋₉ heterocyclylene having 1 to 4 heteroatoms selected from N, O, and S; or A¹ and A², together with -S-S-, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from N, O, and S; optionally substituted C₁₋₉ heterocyclylene having 1 to 4 heteroatoms selected from N, O, and S; O; optionally substituted N; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from N, O, and S;

L is absent or a conjugating group including or consisting of one or more conjugating moieties;

R⁵ is absent, hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, or any combination thereof, where the hydrophilic functional group is optionally protected with a protecting group;

r is an integer from 1 to 10;

where A², A³, and A⁴ combine to form a group having at least three atoms in the shortest chain connecting -S-S-A¹-R⁵ and —X—; and

each R⁴ and R⁶ is independently selected from the group consisting of hydrogen; optionally substituted C₁₋₆ alkyl; optionally substituted C₂₋₇ alkanoyl; hydroxyl; optionally substituted C₁₋₆ alkoxy; optionally substituted C₃₋₈ cycloalkyl; optionally substituted C₃₋₈ cycloalkenyl; optionally substituted C₆₋₁₄ aryl; optionally substituted C₆₋₁₅ aryloyl; optionally substituted C₁₋₉ heterocyclyl having 1 to 4 heteroatoms selected from nitrogen, oxygen, and sulfur; and optionally substituted C₃₋₁₀ (heterocycle)oyl having 1 to 4 heteroatoms selected from nitrogen, oxygen, and sulfur.

Other embodiments of the compound of formula (VII) include the following: Z is absent;

A¹ is selected from the group consisting of a bond, optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted (C₃₋₈ cycloalkyl)-C₁₋₄-alkylene; optionally substituted (C₃₋₈ cycloalkenyl)-C₁₋₄-alkylene; optionally substituted C₆₋₁₄ arylene; optionally substituted (C₆₋₁₄ aryl)-C₁₋₄-alkylene; optionally substituted C₁₋₉ heteroarylene having 1 to 4 heteroatoms selected from nitrogen, oxygen, and sulfur; optionally substituted (C₁₋₉ heteroaryl)-C₁₋₄-alkylene having 1 to 4 heteroatoms selected from nitrogen, oxygen; optionally substituted C₁₋₉ heterocyclylene having 1 to 4 heteroatoms selected from nitrogen, oxygen, and sulfur; and optionally substituted (C₁₋₉ heterocyclyl)-C₁₋₄-alkylene having 1 to 4 heteroatoms selected from nitrogen, oxygen, and sulfur; and A² is selected from the group consisting of optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from nitrogen, oxygen, and sulfur; and optionally substituted C₁₋₉ heterocyclylene having 1 to 4 heteroatoms selected from nitrogen, oxygen, and sulfur; or A¹ and A², together with -S-S-, join to form an optionally substituted 5 to 16 membered ring;

A³ is selected from the group consisting of a bond, optionally substituted C₁₋₆ alkylene; optionally substituted C₃₋₈ cycloalkylene; optionally substituted C₃₋₈ cycloalkenylene; optionally substituted C₆₋₁₄ arylene, optionally substituted C₁₋₉ heteroarylene having 1 to 4 heteroatoms selected from nitrogen, oxygen, and sulfur; optionally substituted C₁₋₉ heterocyclylene having 1 to 4 heteroatoms selected from nitrogen, oxygen, and sulfur; O; NR⁶; and S;

A⁴ is selected from the group consisting of optionally substituted C₁₋₆ alkylene; optionally substituted C₃₋₈ cycloalkylene; and optionally substituted C₁₋₉ heterocyclylene having 1 to 4 heteroatoms selected from nitrogen, oxygen, and sulfur;

L is a bond or a conjugating group including or consisting of one or more conjugating moieties;

R⁵ is absent, hydrogen, optionally substituted C₁₋₆ alkyl, a hydrophilic functional group, or a group comprising an auxiliary moiety selected from the group consisting of a small molecule, a polypeptide, a carbohydrate, a neutral organic polymer, a positively charged polymer, a therapeutic agent, a targeting moiety, an endosomal escape moiety, and combination thereof;

r is an integer from 1 to 10;

where A², A³, and A⁴ combine to form a group having at least three atoms in the shortest chain connecting -S-S- and X; and

each R⁴ is independently hydrogen; optionally substituted C₁₋₆ alkyl; optionally substituted C₂₋₇ alkanoyl; hydroxyl; optionally substituted C₁₋₆ alkoxy; optionally substituted C₃₋₈ cycloalkyl; optionally substituted C₃₋₈ cycloalkenyl; optionally substituted C₆₋₁₄ aryl; optionally substituted C₆₋₁₅ aryloyl; optionally substituted C₂₋₉ heterocyclyl having 1 to 4 heteroatoms selected from nitrogen, oxygen, and sulfur; or optionally substituted C₃₋₁₀ (heterocycle)oyl having 1 to 4 heteroatoms selected from nitrogen, oxygen, and sulfur.

In yet other embodiments of the compound of formula (VII) -A¹-S-S-A²-A³-A⁴- or -S-S-A²-A³-A⁴- group is one of the following:

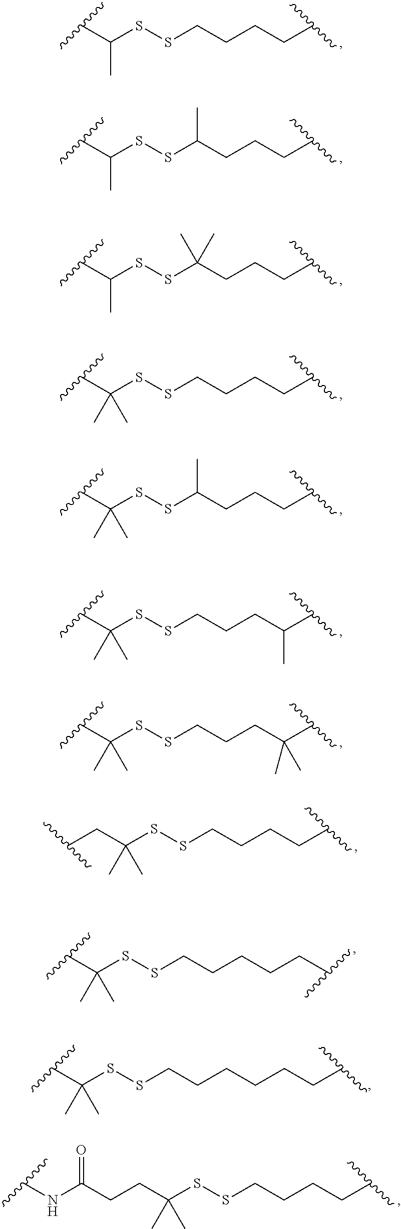

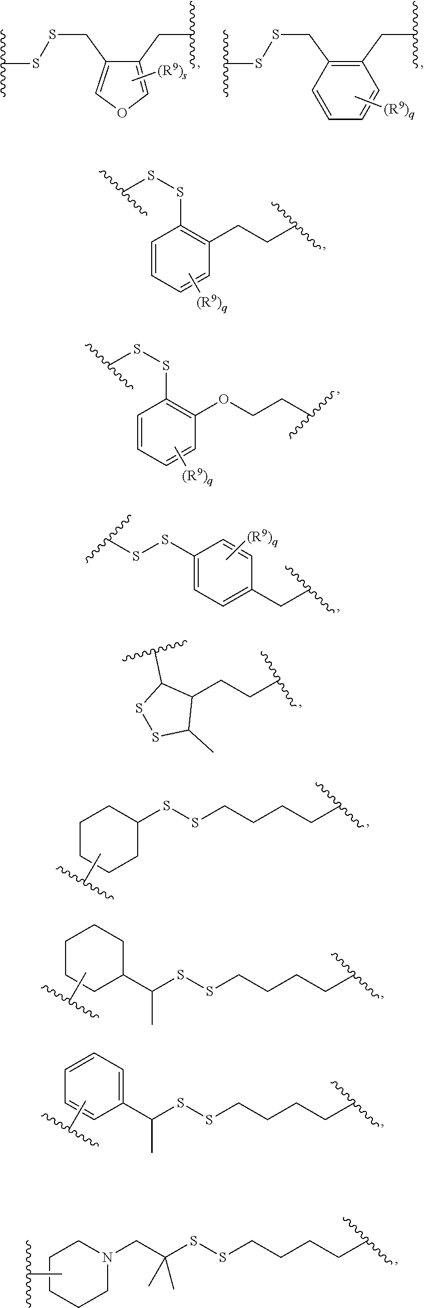

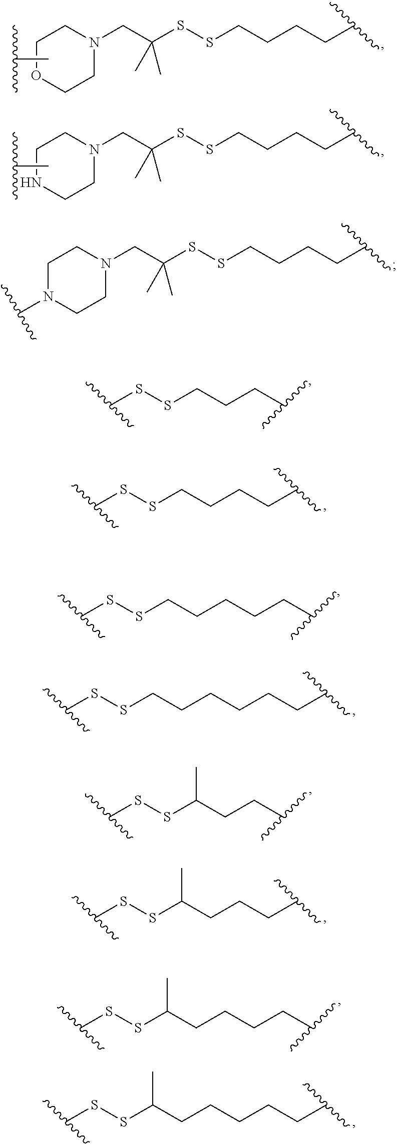

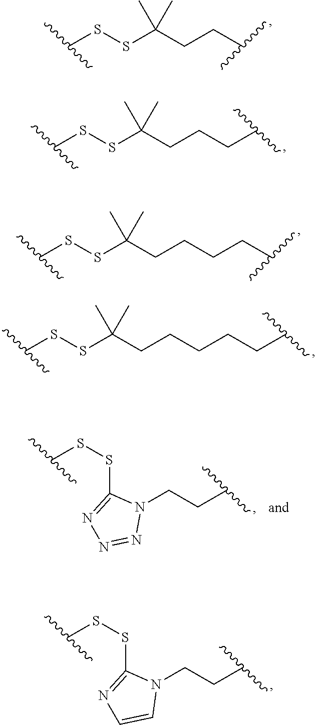

where

each R⁹ is, independently, halo, optionally substituted C₁₋₆ alkyl; optionally substituted C₂₋₆ alkenyl; optionally substituted C₂₋₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from nitrogen, oxygen, and sulfur; optionally substituted (C₁₋₉ heteroaryl)-C₁₋₄-alkyl having 1 to 4 heteroatoms selected from nitrogen, oxygen; optionally substituted C₁₋₉ heterocyclyl having 1 to 4 heteroatoms selected from nitrogen, oxygen, and sulfur; optionally substituted (C₁₋₉ heterocyclyl)-C₁₋₄-alkyl having 1 to 4 heteroatoms selected from nitrogen, oxygen, and sulfur; amino; or optionally substituted C₁₋₆ alkoxy; or two adjacent R⁹ groups, together with the atoms to which each the R⁹ is attached, combine to form a cyclic group selected from the group consisting of C aryl, C₂₋₆ heterocyclyl, or C₂₋₅ heteroaryl, where the cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4; and

s is 0, 1, or 2.

In still other embodiments, the bioreversible group contains one of the following structures:

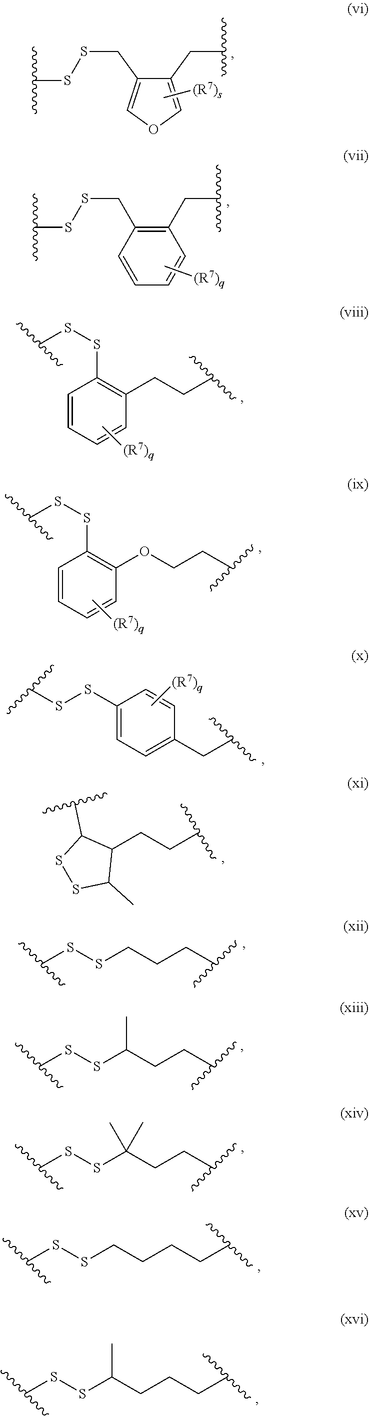

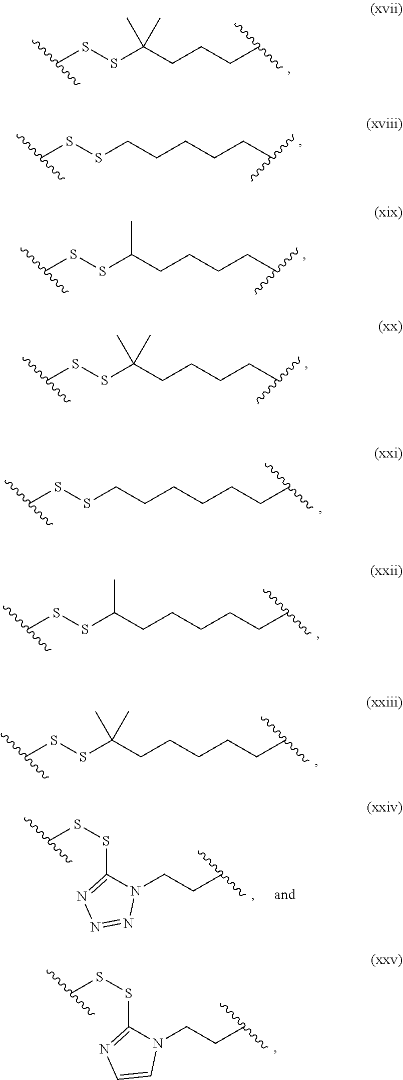

where

each R⁷ is independently C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)SO₂NR^{E}R^{F}, where an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl; or two adjacent R⁷ groups, together with the atoms to which each the R⁷ is attached combine to form a cyclic group selected from the group consisting of C aryl, C₂₋₅ heterocyclyl, or C₂-heteroaryl, where the cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C-10 aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen,-6 alkyl, C-10 aryl, and (C₆₋₁₀ aryl)-C₁₋₄-alkyl; —(CH₂)SO₂R^{D}, where q is an integer from zero to four and where R^{D} is selected from the group consisting of C₁₋₆ alkyl, C₆₋₁₀ aryl, and C₆₋₁₀ aryl)-1-4-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

q is 0, 1, 2, 3, or 4; and

s is 0, 1, or 2.

In particular embodiments, the auxiliary moiety can be attached to the group containing a disulfide linkage by forming one or more covalent bonds to a conjugating moiety found in the conjugating group.

Conjugates

Nucleotide constructs of the invention may contain one or more conjugating groups having one or more conjugating moieties. The conjugating moieties can in turn be used to attach various other auxiliary moieties, e.g., a small molecule, a polypeptide, a carbohydrate, a neutral organic polymer, a positively charged polymer, a therapeutic agent, a targeting moiety, an endosomal escape moiety, or combination thereof, to the nucleotide construct. In a certain embodiment, more than one type of conjugating moiety is present in a nucleotide construct, thereby allowing the selective and/or sequential coupling of auxiliary moieties to the nucleotide construct. The location of attachment in a polynucleotide construct is determined by the use of the appropriate nucleotide construct in the synthesis of the polymer. A nucleotide construct containing one more conjugating moieties will react, under appropriate conditions, with one or more corresponding conjugating moieties on auxiliary moieties. The auxiliary moiety may intrinsically possess the conjugating moiety, e.g., terminal or lysine amine groups and thiol groups in peptides or polypeptides, or it may be modified to include a small linking group to introduce the conjugating moiety. Introduction of such linking groups is well known in the art. It will be understood that an auxiliary moiety attached to a nucleotide construct of the invention includes any necessary linking group.

Diverse bond-forming methods can be used to conjugate the auxiliary moiety to the nucleotide constructs described herein. Exemplary reactions include: Huisgen cycloaddition between an azide and an alkyne to form a triazole; the Diels-Alder reaction between a dienophile and a diene/hetero-diene; bond formation via other pericyclic reactions such as the ene reaction; amide or thioamide bond formation; sulfonamide bond formation; alcohol or phenol alkylation (e.g., with diazo compounds), condensation reactions to form oxime, hydrazone, or semicarbazide group, conjugate addition reactions by nucleophiles (e.g., amines and thiols), disulfide bond formation, and nucleophilic substitution at a carboxylic functionality (e.g., by an amine, thiol, or hydroxyl nucleophile). Other exemplary methods of bond formation are described herein and known in the art.

Nucleophile/Electrophile Reactions

Nucleophiles and electrophiles can engage in bond forming reactions selected from, without limitation, insertion by an electrophile into a C—H bond, insertion by an electrophile into an O—H bond, insertion by an electrophile into an N—H bond, addition of the electrophile across an alkene, addition of the electrophile across an alkyne, addition to electrophilic carbonyl centers, substitution at electrophilic carbonyl centers, addition to ketenes, nucleophilic addition to isocyanates, nucleophilic addition to isothiocyanates, nucleophilic substitution at activated silicon centers, nucleophilic displacement of an alkyl halide, nucleophilic displacement at an alkyl pseudohalide, nucleophilic addition/elimination at an activated carbonyl, 1,4-conjugate addition of a nucleophile to an α, β-unsaturated carbonyl, nucleophilic ring opening of an epoxide, nucleophilic aromatic substitution of an electron deficient aromatic compound, a nucleophilic addition to activated phosphorus centers, nucleophilic substitution at activated phosphorous centers, nucleophilic addition to activated sulfur centers, and nucleophilic substitution at activated sulfur centers.

A nucleophilic conjugating moiety may be selected from optionally substituted alkenes, optionally substituted alkynes, optionally substituted aryl, optionally substituted heterocyclyl, hydroxyl groups, amino groups, alkylamino groups, anilido groups, and thio groups.

An electrophilic conjugating moiety may be selected from nitrenes, nitrene precursors such as azides, carbenes, carbene precursors, activated silicon centers, activated carbonyls, anhydrides, isocyanates, thioisocyanates, succinimidyl esters, sulfosuccinimidyl esters, maleimides, alkyl halides, alkyl pseudohalides, epoxides, episulfides, aziridines, electron-deficient aryls, activated phosphorus centers, and activated sulfur centers.

For example, conjugation can occur via a condensation reaction to form a linkage that is a hydrazone bond.

Conjugation via the formation of an amide bond can be mediated by activation of a carboxyl-based conjugating moiety and subsequent reaction with a primary amine-based conjugating moiety. Activating agents can be various carbodiimides like: EDC (1-Ethyl-3-(3-dimethylaminopropyl)carbodiimide hydrochloride), EDAC (1-ethyl-3(3-dimethylaminopropyl)carbodiimide hydrochloride), DCC (dicyclohexyl carbodiimide), CMC (1-Cyclohexyl-3-(2-morpholinoethyl) carbodiimide), DIC (diisopropyl carbodiimide) or Woodward's reagent K (N-ethyl-3-phenylisoxazolium-3′-sulfonate). Reaction of an activated NHS-Ester-based conjugating moiety with a primary amine-based conjugating moiety also results in formation of an amide bond.

The nucleotide construct may contain a carbonyl-based conjugating moiety. Conjugation via the formation of a secondary amine can be achieved by reacting an amine-based conjugating moiety with an aldehyde-based conjugating moiety, followed by reducing with a hydride donor like sodium cyanoborohydride. Aldehyde-based conjugating moieties can be introduced for instance by oxidation of sugar moieties or by reaction with SFB (succinimidyl-p-formyl benzoate) or SFPA (succinimidyl-p-formylphenoxyacetate).

Ether formation can also be used to conjugate auxiliary moieties to the nucleotide constructs of the invention. Conjugation via ether linkages can be mediated by reaction of an epoxide-based conjugating moiety with a hydroxy-based conjugating moiety.

Thiols can also be used as conjugating moieties. For example, conjugation via the formation of disulfide bonds can be accomplished by pyridyldisulfide mediated thiol-disulfide exchange. Introduction of sulfhydryl-based conjugating moieties is mediated for instance by Traut's Reagent (2-iminothiolane) SATA (N-succinimidyl S-acetylthioacetate, SATP (succinimidyl acetylthiopropionate), SPDP (N-succinimidyl 3-(2-pyridyldithio)propionate, SMPT (succinimidyloxycarbonyl-α-methyl-α-(2-pyridyldithio)toluene), N-acetylhomocysteinethiolactone, SAMSA (S-acetylmercaptosuccinic anhydride), AMBH (2-Acedamido-4-mercaptobuturic acid hydrazide), and cystamine (2,2′-dithiobis(ethylamine).

Conjugation via the formation of thioether linkages can be performed by reacting a sulfhydryl based conjugating moieties with maleimide- or iodoacetyl-based conjugating moieties or by reacting with epoxide-based conjugating moieties. Maleimide -based conjugating moieties can be introduced by SMCC (succinimidyl-4-(N-maleimidomethyl)cyclohexane-1-carboxylate), sulfo-SMCC (sulfosuccinimidyl 4-(N-maleidomethyl)-cyclohexane-1-carboxylate), MBS (m-Maleimidobenzoyl-N-hydroxysuccinimide ester), sulfo-MBS (m-Maleimidobenzoyl-N-sulfohydroxy succinimide ester), SMPB (Succinimidyl-4-(p-maleidophenyl)butyrate), sulfo-SMPB (sulfosuccinimidyl 4-(p-maleimidophenyl)butyrate), GMBS (N-α-maleimidobuturyl-oxysuccinimide ester), sulfo GMBS (N-α-maleimidobuturyl-oxysulfosuccinimide ester).

Thiol-based conjugating moieties can also react with iodoacetyl-based conjugating moieties. Iodoacetyl-based conjugating moieties can be inserted with SIAB (N-succinimidyl(4-iodoacetyl)aminobenzoate, sulfo SIAB (sulfo-succinimidyl(4-iodoacetyl)-aminobenzoate), SIAX (succinimidyl6-[(iodoacetyl-amino]hexanoate), SIAXX (succinimidyl6-[6-(((iodoacetyl)amino)-hexanoyl)amino]hexanoate), SIAC (succinimidyl 4-(((iodoacetyl)amino)methyl)-cyclohexane-1-carboxylate), SIACX (succinimidyl 6-((((4-(iodoacetyl)amino)methyl)-cyclohexane-1-carbonyl)amino) hexanoate), and NPIA (p-nitrophenyl iodoacetate).

Conjugation via the formation of a carbamate linkage can be performed by reaction of a hydroxy-based conjugating moiety with CDI (N,N′-carbonyldiimidazole) or DSC (N,N′-disuccinimidyl carbonate) or N-hydroxysuccinimidylchloroformate and subsequent reaction with an amine-based conjugating moiety.

Photolytic and Thermolytic Conjugation

Alternatively, the conjugating moiety can employ photolytic or thermolytic activation in order to form the desired covalent bond. Conjugating moieties that include azido functionality are one example. Thus, conjugation can also be achieved by the introduction of a photoreactive conjugating moiety. Photoreactive conjugating moieties are aryl azides, halogenated aryl azides, benzophenones certain diazo compounds and diazirine derivatives. They react with amino-based conjugating moieties or with conjugating moieties that have activated hydrogen bonds.

The azido-based conjugating moieties are UV labile and, upon photolysis, can lead to the formation of nitrene electrophiles that can react with nucleophilic conjugating moieties such as aryl-based conjugating moieties or alkenyl-based conjugating moieties. Alternatively, the heating of these azido compounds can also result in nitrene formation.

Cycloaddition Reactions

Cycloaddition reactions can be used to form the desired covalent bond. Representative cycloaddition reactions include, but are not limited to, the reaction of an alkene-based conjugating moiety with a 1,3-diene-based conjugating moiety (Diels-Alder reaction), the reaction of an alkene-based conjugating moiety with an α,β-unsaturated carbonyl-based conjugating moiety (hetero Diels-Alder reaction), and the reaction of an alkyne-based conjugating moiety with an azido-based conjugating moiety (Hüisgen cycloaddition). Selected, non-limiting examples of conjugating moieties that include reactants for cycloaddition reactions are: alkenes, alkynes, 1,3-dienes, α,β-unsaturated carbonyls, and azides. For example, the Huisgen cycloaddition (click reaction) between azides and alkynes has been used for the functionalization of diverse biological entities.

Coupling Reactions

Conjugating moieties also include, but are not limited to, reactants for hydrosilylation, olefin cross-metathesis, conjugate addition, Stille coupling, Suzuki coupling, Sonogashira coupling, Hiyama coupling, and Heck reaction. Conjugation moieties for these reactions include hydridosilanes, alkenes (e.g., activated alkenes, such as enones or enoates), alkynes, aryl halides, aryl pseudohalides (e.g., triflates or nonaflates), alkyl halides, and alkyl pseudohalides (e.g., triflates, nonaflates, and phosphates). Catalysts for cross-coupling reactions are well-known in the art. Such catalysts may be organometallic complexes or metal salts (e.g., Pd(0), Pd(II), Pt(0), Pt(II), Pt(IV), Cu(I), or Ru(II)). Additives, such as ligands (e.g., PPh₃, PCy₃, BINAP, dppe, dppf, SIMes, or SIPr) and metal salts (e.g., LiCl), may be added to facilitate cross-coupling reactions.

Auxiliary Moieties for Conjugation

Various auxiliary moieties can be conjugated to the nucleotide constructs of the invention (e.g., siRNA), and the auxiliary moieties can have any number of biological or chemical effects. Biological effects include, but are not limited to, inducing intracellularization, binding to a cell surface, targeting a specific cells type, allowing endosomal escape, altering the half-life of the polynucleotide in vivo, and providing a therapeutic effect. Chemical effects include, but are not limited to, changing the solubility, charge, size, and reactivity.

Small Molecules

Small molecule-based auxiliary moieties (e.g., organic compounds having molecular weights of 1000 Da or less) can be conjugated to nucleotide constructs of the invention. Examples of such small molecules include, but are not limited to, substituted or unsubstituted alkanes, alkenes, or alkynes, e.g., hydroxy-substituted, NH₂-substituted, mono-, di-, or trialkyl amino substituted, guanidino substituted, heterocyclyl substituted, and protected versions thereof. Other small molecules include steroids (e.g., cholesterol), other lipids, bile, and amino acids. A small molecule may be added to a polynucleotide to provide neutral or positive charge or to alter the hydrophilicity or hydrophobicity of the polynucleotide.

Polypeptides

A polypeptide (including a fusion polypeptide) refers to a polymer in which the monomers are amino acid residues which are joined together through amide bonds. When the amino acids are alpha-amino acids, either the L-optical isomer or the D-optical isomer can be used. A polypeptide encompasses an amino acid sequence and includes modified sequences such as glycoproteins, retro-inverso polypeptides, D-amino acid and the like. A polypeptide includes naturally occurring proteins, as well as those which are recombinantly or synthetically synthesized. A polypeptide may include more than one domain have a function that can be attributed to the particular fragment or portion of a polypeptide. A domain, for example, includes a portion of a polypeptide which exhibits at least one useful epitope or functional domain. Two or more domains may be functionally linked such that each domain retains its function yet includes a single peptide or polypeptide (e.g., a fusion polypeptide). For example, a functional fragment of a PTD includes a fragment which retains transduction activity. Biologically functional fragments, for example, can vary in size from a fragment as small as an epitope capable of binding an antibody molecule, to a large polypeptide capable of participating in the characteristic induction or programming of phenotypic changes within a cell.

In some embodiments, retro-inverso polypeptides are used. “Retro-inverso” means an amino-carboxy inversion as well as enantiomeric change in one or more amino acids (i.e., levorotatory (L) to dextrorotatory (D)). A polypeptide of the invention encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995). The overall structural features of a retro-inverso polypeptide are similar to those of the parent L-polypeptide. The two molecules, however, are roughly mirror images because they share inherently chiral secondary structure elements. Main-chain peptidomimetics based on peptide-bond reversal and inversion of chirality represent important structural alterations for peptides and proteins, and are highly significant for biotechnology. Antigenicity and immunogenicity can be achieved by metabolically stable antigens such as all-D- and retro-inverso-isomers of natural antigenic peptides and polypeptide. Several PTD-derived peptidomimetics are provided herein.

Polypeptides and fragments can have the same or substantially the same amino acid sequence as the naturally derived polypeptide or domain. “Substantially identical” means that an amino acid sequence is largely, but not entirely, the same, but retains a functional activity of the sequence to which it is related. An example of a functional activity is that the fragment is capable of transduction, or capable of binding to an RNA. For example, fragments of full length TAT are described herein that have transduction activity. In general two peptides, polypeptides or domains are “substantially identical” if their sequences are at least 85%, 90%, 95%, 98% or 99% identical, or if there are conservative variations in the sequence. A computer program, such as the BLAST program (Altschul et al., 1990) can be used to compare sequence identity.

A polypeptide of the invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the backbone, the amino acid side-chains and the amino or carboxyl termini. It will be appreciated that the same type of modification may be present in the same or varying degrees at several sites in a given peptide or polypeptide. Also, a given polypeptide may contain many types of modifications. A polypeptide may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann N.Y. Acad Sci 663:48-62 (1992)).

A polypeptide domain or a fusion polypeptide of the invention can be synthesized by commonly used methods such as those that include t-BOC or FMOC protection of alpha-amino groups. Both methods involve stepwise synthesis in which a single amino acid is added at each step starting from the C-terminus of the peptide or polypeptide (See, Coligan, et al., Current Protocols in Immunology, Wiley Interscience, 1991, Unit 9). Polypeptides of the invention can also be synthesized by the well known solid phase peptide synthesis methods such as those described by Merrifield, J. Am. Chem. Soc., 85:2149, 1962; and Stewart and Young, Solid Phase Peptides Synthesis, Freeman, San Francisco, 1969, pp. 27-62, using a copoly(styrene-divinylbenzene) containing 0.1-1.0 mMol amines/g polymer. On completion of chemical synthesis, the polypeptides can be deprotected and cleaved from the polymer by treatment with liquid HF-10% anisole for about ¼-1 hours at 0° C. After evaporation of the reagents, the polypeptides are extracted from the polymer with a 1% acetic acid solution, which is then lyophilized to yield the crude material. The polypeptides can be purified by such techniques as gel filtration on Sephadex G-15 using 5% acetic acid as a solvent. Lyophilization of appropriate fractions of the column eluate yield homogeneous peptide or polypeptide, which can then be characterized by standard techniques such as amino acid analysis, thin layer chromatography, high performance liquid chromatography, ultraviolet absorption spectroscopy, molar rotation, or measuring solubility. If desired, the polypeptides can be quantified by the solid phase Edman degradation.

Carbohydrates

Carbohydrate-based auxiliary moieties that can be attached to the nucleotide constructs of the invention include monosaccharides, disaccharides, and polysaccharides. Examples include allose, altrose, arabinose, cladinose, erythrose, erythrulose, fructose, D-fucitol, L-fucitol, fucosamine, fucose, fuculose, galactosamine, D-galactosaminitol, N-acetyl-galactosamine, galactose, glucosamine, N-acetyl-glucosamine, glucosaminitol, glucose, glucose-6-phosphate gulose glyceraldehyde, L-glycero-D-mannos-heprose, glycerol, glycerone, gulose idose, lyxose, mannosamine, mannose, mannose-6-phosphate, psicose, quinovose, quinovosamine, rhamnitol, rhamnosamine, rhamnose, ribose, ribulose, sedoheptulose, sorbose, tagatose, talose, tararic acid, threose, xylose and xylulose. A monosaccharide can be in D- or L-configuration. A monosaccharide may further be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), a imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group, e.g., glucitol), aldonic acid (aldehydic group replaced by carboxy group), a ketoaldonic acid, a uronic acid, an aldaric acid, and so forth. Amino sugars include amino monosaccharides, such as galactosamine, glucosamine, mannosamine, fucosmine, quinavosamine, neuraminic acid, muramic acid, lactosediamine, acosamine, bacillosamine, daunosamine, desosamine, forosamine, garosamine, kanosamine, kanosamine, mycaminose, myosamine, persosamine, pneumosamine, purpurosamine, rhodosmine. It is understood that the monosaccharide and the like can be further substituted. Di- and polysaccharides include abequose, acrabose, amicetose, amylopectin, amylose, apiose, arcanose, ascarylose, ascorbic acid, boivinose, cellobiose, cellotriose, cellulose, chacotriose, chalcose, chitin, colitose, cyclodextrin, cymarose, dextrin, 2-deoxyribose, 2-deoxyglucose diginose, digitalose, digitoxose, evalose, evemitrose, fructooligosaccharide, galacto-oligosaccharide, gentianose, genitiobiose, glucan, gluicogen, glycogen, hamamelose, heparin, inulin, isolevoglucosenone, isomaltose, isomaltotriose, isopanose, kojibiose, lactose, lactosamine, lactosediamine, laminarabiose, levoglucosan, levoglucosenone, β-maltose, maltriose, mannan-oligosaccharide, manninotriose, melezitose, melibiose, muramic acid, mycarose, mycinose, neuraminic acid, migerose, nojirimycon, noviose, oleandrose, panose, paratose, planteose, primeverose, raffinose, rhodone, rutinose, oleandrose, panose, paratose, planteose, primeverose, raffinose, rhodinose, rutinose, sarmentose, sedoheptulose, sedoheptulosan, solatriose, sophorose, stachyose, streptose, sucrose, α,α-trehalose, trahalosamine, turanose, tyvelose, xylobiose, umbelliferose and the like. A carbohydrate can serve one or more functions in polynucleotide constructs of the invention, e.g., a carbohydrate can be a targeting moiety (e.g., mannose) or can improve solubility of the polynucleotide construct in aqueous media (e.g., glucitol).

Polymers

The nucleotide constructs described herein can also include covalently attached neutral or charged (e.g., cationic) polymer-based auxiliary moieties. Examples of positively charged polymers include poly(ethylene imine) (PEI), spermine, spermidine, and poly(amidoamine) (PAMAM). Neutral polymers include poly(C₁₋₆ alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(acrylic acid), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, poly(2-ethylacrylic acid), N-isopropylacrylamide polymers, polyphosphazine and poly(N,N-dialkylacrylamides). Exemplary polymer auxiliary moieties may have molecular weights of less than 100, 300, 500, 1000, or 5000. Other polymers are known in the art.

Therapeutic Agents

Therapeutic agents, which include diagnostic/imaging agents, can be covalently attached as auxiliary moieties to the nucleotide constructs of the invention or can be administered as a co-therapy as described herein. They can be naturally occurring compounds, synthetic organic compounds, or inorganic compounds. Exemplary therapeutic agents include, but are not limited to, antibiotics, antiproliferative agents, rapamycin macrolides, analgesics, anesthetics, antiangiogenic agents, vasoactive agents, anticoagulants, immunomodulators, cytotoxic agents, antiviral agents, antithrombotic drugs, antibodies, neurotransmitters, psychoactive drugs, and combinations thereof. Additional examples of therapeutic agents include, but are not limited to, cell cycle control agents; agents which inhibit cyclin protein production; cytokines, including, but not limited to, Interleukins 1 through 13 and tumor necrosis factors; anticoagulants, anti-platelet agents; TNF receptor domains and the like. Typically the therapeutic agent is neutral or positively charged. In certain instances, where the therapeutic agent is negatively charged, an additional charge neutralization moiety (e.g., a cationic peptide) can be used.

A therapeutic moiety can be linked as an auxiliary moiety to a nucleotide construct disclosed herein to allow for diagnostic assay/imaging. Examples of such moieties include, but are not limited to, detectable labels, such as an isotope, a radioimaging agent, a marker, a tracer, a fluorescent label (e.g., rhodamine), and a reporter molecule (e.g., biotin).

Exemplary diagnostic agents include, but are not limited to, imaging agents, such as those that are used in positron emission tomography (PET), computer assisted tomography (CAT), single photon emission computerized tomography, X-ray, fluoroscopy, and magnetic resonance imaging (MRI). Suitable materials for use as contrast agents in MRI include, but are not limited to, gadolinium chelates, as well as iron, magnesium, manganese, copper, and chromium chelates. Examples of materials useful for CAT and X-rays include, but are not limited to, iodine based materials.

Examples of radioimaging agents emitting radiation (detectable radio-labels) that may be suitable are exemplified by indium-111, technitium-99, or low dose iodine-131. Detectable labels, or markers, for use in conjunction with or attached to the nucleotide constructs of the invention as auxiliary moieties may be a radiolabel, a fluorescent label, a nuclear magnetic resonance active label, a luminescent label, a chromophore label, a positron emitting isotope for PET scanner, a chemiluminescence label, or an enzymatic label. Fluorescent labels include, but are not limited to, green fluorescent protein (GFP), fluorescein, and rhodamine. The label may be for example a medical isotope, such as for example and without limitation, technetium-99, iodine-123 and -131, thallium-201, gallium-67, fluorine-18, indium-111, etc.

Other therapeutic agents known in the art can likewise be used in conjunction with, or attached to the nucleotide constructs of the invention as auxiliary moieties.

Targeting Moieties

The invention provides for one or more targeting moieties which can be attached to a nucleotide construct disclosed herein as an auxiliary moiety, for example as a targeting auxiliary moiety. A targeting moiety (e.g., extracellular targeting moiety) is selected based on its ability to target constructs of the invention to a desired or selected cell population that expresses the corresponding binding partner (e.g., either the corresponding receptor or ligand) for the selected targeting moiety. For example, a construct of the invention could be targeted to cells expressing epidermal growth factor receptor (EGFR) by selected epidermal growth factor (EGF) as the targeting moiety. Alternatively, the targeting moiety (e.g., intracellular targeting moiety) can target constructs of the invention to a desired site within the cell (e.g., endoplasmic reticulum, Golgi apparatus, nucleus, or mitochondria). Non-limiting examples of the intracellular targeting moieties include compounds P38 and P39 of Table 3 and peptide fragments thereof (i.e., MKWVTFISLLFLFFSSAYS (SEQ ID NO:56) and MIRTLLLSTLVAGALS (SEQ ID NO:57), respectively).

A polynucleotide construct of the invention, thus, may include one or more targeting moieties selected from the group constisting of intracellular targeting moieties, extracellular targeting moieties, and combinations thereof. Thus, the inclusion of one or more extracellular targeting moieties (e.g., each extracellular targeting moiety independently selected from the group consisting of folate, mannose, galactosamine (e.g., N-acetyl galactosamine), and prostate specific membrane antigen) and one or more intracellular targeting moiety (e.g., a moiety targeting endoplasmic reticulum, Golgi apparatus, nucleus, or mitochondria) in the polynucleotide construct of the invention can facilitate the delivery of the polynucleotides to a specific site within the specific cell population. In some embodiments, the targeting moiety contains one or more mannose carbohydrates. Mannose targets the mannose receptor, which is a 175 KDa membrane-associated receptor that is expressed on sinusoidal liver cells and antigen presenting cells (e.g., macrophages and dendritic cells). It is a highly effective endocytotic/recycling receptor that binds and internalizes mannosylated pathogens and proteins (Lennartz et. al. J. Biol. Chem. 262:9942-9944,1987; Taylor et. al. J. Biol. Chem. 265:12156-62, 1990).

Some of the extracellular targeting moieties of the invention are described herein. In one embodiment, the targeting moiety is a receptor binding domain. In another embodiment, the targeting moiety is or specifically binds to a protein selected from the group including insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), Lewisy, MUC-1, epithelial cell adhesion molecule (EpCAM), cancer antigen 125 (CA125), prostate specific membrane antigen (PSMA), TAG-72 antigen, and fragments thereof. In further embodiments, the targeting moiety is erythroblastic leukemia viral oncogene homolog (ErbB) receptor (e.g., ErbB1 receptor; ErbB2 receptor; ErbB3 receptor; and ErbB4 receptor). In other embodiments, a targeting moiety may selectively bind to asialoglycoprotein receptor, a manno receptor, or a folate receptor. In particular embodiments, the targeting moiety contains one or more N-acetyl galactosamines (GaINAc), mannoses, or a folate ligand. In certain embodiments, the folate ligand has the structure:

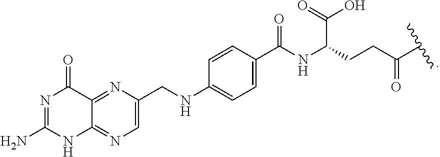 [SMILES: *C(=O)CC[C@H](NC(=O)c1ccc(NCc2cnc3[nH]c(N)nc(=O)c3n2)cc1)C(=O)O]

The targeting moiety can also be selected from bombesin, gastrin, gastrin-releasing peptide, tumor growth factors (TGF), such as TGF-α and TGF-β, and vaccinia virus growth factor (VVGF). Non-peptidyl ligands can also be used as the targeting moiety and may include, for example, steroids, carbohydrates, vitamins, and lectins. The targeting moiety may also be selected from a polypeptide, such as somatostatin (e.g., a somatostatin having the core sequence cyclo[Cys-Phe-D-Trp-Lys-Thr-Cys] (SEQ ID NO:103), and in which, for example, the C-terminus of the somatostatin analog is: Thr-NH₂), a somatostatin analog (e.g., octreotide and lanreotide), bombesin, a bombesin analog, or an antibody, such as a monoclonal antibody.

Other peptides or polypeptides for use as a targeting auxiliary moiety in nucleotide constructs of the invention can be selected from KiSS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, peptides from complementarity determining regions of an antitumor antibody, glutathione, YIGSR (SEQ ID NO:104) (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, endothelin, leukotriene B4 (LTB4), chemotactic peptides (e.g., N-formyl-methionyl-leucyl-phenylalanine-lysine (fMLFK; SEQ ID NO:105), GP IIb/IIIa receptor antagonists (e.g., DMP444), human neutrophil elastase inhibitor (EPI-HNE-2 and EPI-HNE-4), plasmin inhibitor, antimicrobial peptides, apticide (P280 and P274), thrombospondin receptor (including analogs such as TP-1300), bitistatin, pituitary adenylyl cyclase type I receptor (PAC1), fibrin α-chain, peptides derived from phage display libraries (e.g., SEQ ID NOs: 13 and 14), and conservative substitutions thereof.

Immunoreactive ligands for use as a targeting moiety in nucleotide constructs of the invention include an antigen-recognizing immunoglobulin (also referred to as “antibody”), or antigen-recognizing fragment thereof. As used herein, “immunoglobulin” refers to any recognized class or subclass of immunoglobulins such as IgG, IgA, IgM, IgD, or IgE. Typical are those immunoglobulins which fall within the IgG class of immunoglobulins. The immunoglobulin can be derived from any species. Typically, however, the immunoglobulin is of human, murine, or rabbit origin. In addition, the immunoglobulin may be polyclonal or monoclonal, but is typically monoclonal.

Targeting moieties of the invention may include an antigen-recognizing immunoglobulin fragment. Such immunoglobulin fragments may include, for example, the Fab′, F(ab′)₂, Fv or Fab fragments, single-domain antibody, ScFv, or other antigen-recognizing immunoglobulin fragments. Fc fragments may also be employed as targeting moieties. Such immunoglobulin fragments can be prepared, for example, by proteolytic enzyme digestion, for example, by pepsin or papain digestion, reductive alkylation, or recombinant techniques. The materials and methods for preparing such immunoglobulin fragments are well-known to those skilled in the art. See Parham, J. Immunology, 131, 2895, 1983; Lamoyi et al., J. Immunological Methods, 56, 235, 1983.

Targeting moieties of the invention include those targeting moieties which are known in the art but have not been provided as a particular example in this disclosure.

Endosomal Escape

The invention provides for one or more endosomal escape moieties which can be attached to a nucleotide construct disclosed herein as an auxiliary moiety, for example, as an endosomal escape auxiliary moiety. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-amino ester)s; peptides or polypeptides such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Exemplary endosomal escape moieties are provided in Table 3 in Example 1.

Delivery Domain

The invention provides for one or more delivery domain moieties which can be attached to a nucleotide construct disclosed herein as an auxiliary moiety, for example as an delivery domain auxiliary moiety. A delivery domain is a moiety that induces transport of a polynucleotide of the invention into a cell, by any mechanism. Typically, nucleotide constructs of the invention will be internalized by macropinocytosis, phagocytosis, or endocytosis (e.g., clathrin-mediated endocytosis, caveolae-mediated endocytosis, and lipid-raft dependent endocytosis), see, e.g., Chem. Soc. Rev., 2011, 40, 233-245. Delivery domains may include peptides or polypeptides (e.g., peptide transduction domains), carbohydrates (hyaluronic acid), and positively charged polymers (poly(ethylene imine), as described herein.

Peptide Transduction Domains

Cellular delivery can be accomplished by macromolecule fusion of “cargo” biological agents (in this case the polynucleotide) to a cationic Peptide Transduction Domain (PTD; also termed Cell Penetrating Peptide (CPP)) such as TAT (SEQ ID NO: 1) or Arge (SEQ ID NO: 2) (Snyder and Dowdy, 2005, Expert Opin. Drug Deliv. 2, 43-51). PTDs can be used to deliver a wide variety of macromolecular cargo, including the polynucleotides described herein (Schwarze et al., 1999, Science 285, 1569-1572; Eguchi et al., 2001, J. Biol. Chem. 276, 26204-26210; and Koppelhus et al., 2002, Antisense Nucleic Acid Drug Dev. 12, 51-63). Cationic PTDs enter cells by macropinocytosis, a specialized form of fluid phase uptake that all cells perform.

Biophysical studies on model vesicles suggest that cargo escape from macropinosome vesicles into the cytoplasm, thus requiring a pH decrease (Magzoub et al., 2005, Biochemistry 44, 14890-14897). The cationic charge of the PTDs is essential for the molecules to traverse the cell membrane. Not surprisingly, conjugation of cationic PTDs (6-8 positive charges) to anionic siRNAs (˜40 negative charges) results in charge neutralization and inactivation of the PTD with no siRNA entering the cells (Turner et al., Blood Cells Mol. Dis., 38(1):1-7, 2007). However, chemical conjugation of cationic PTDs to a nucleotide construct described herein (e.g., anionic RNA or DNA) still results in the nucleotide construct being able to be taken up by cells, and therefore the novel and nonobvious nucleotide constructs disclosed herein do not suffer from any charge neutralization deleterious artifacts seen with other similar methods. Further, cleavage of these PTDs intracellularly allows the polynucleotide to be irreversibly delivered to the targeted cell.

The discovery of several proteins which could efficiently pass through the plasma membrane of eukaryotic cells has led to the identification of a class of proteins from which peptide transduction domains have been derived. The best characterized of these proteins are the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), and more recently the cationic N-terminal domain of prion proteins. Exemplary PTD sequences are provided in Table 1. The invention further provides for one or more of the PTDs listed in Table 1 or other PTDs known in the art (see, e.g., Joliot et al., Nature Cell Biology, 6(3):189-196, 2004) to be conjugated to the nucleotide constructs disclosed herein as auxiliary moieties. Strategies for conjugation include the use of a bifunctional linker that includes a functional group that can be cleaved by the action of an intracellular enzyme.

TABLE 1

| PTD | Sequence | SEQ ID NO. |
|---|---|---|
| TAT | RKKRRQRRR | SEQ ID |
| | | NO.: 1 |
| Penetratin | RQIKIWFQNRRMKWKK | SEQ ID |
| | | NO.: 3 |
| Buforin II | TRSSRAGLQFPVGRVHRLLRK | SEQ ID |
| | | NO.: 4 |
| Transportan | GWTLNSAGYLLGKINKALAALAKKIL | SEQ ID |
| | | NO.: 5 |
| MAP (model | KLALKLALKALKAALKLA | SEQ ID |
| amphipathic | | NO.: 6 |
| peptide) | | |
| K-FGF | AAVALLPAVLLALLAP | SEQ ID |
| | | NO.: 7 |
| Ku70 | VPMLK - PMLKE | SEQ ID |
| | | NO.: 8 |
| Prion | MANLGYWLLALFVTMWTDVGLCKKRPKP | SEQ ID |
| | | NO.: 9 |
| pVEC | LLIILRRRIRKQAHAHSK | SEQ ID |
| | | NO.: 10 |
| Pep-1 | KETWWETWWTEWSQPKKKRKV | SEQ ID |
| | | NO.: 11 |
| SynB1 | RGGRLSYSRRRFSTSTGR | SEQ ID |
| | | NO.: 12 |
| Pep-7 | SDLWEMMMVSLACQY | SEQ ID |
| (phage | | NO.: 13 |
| display) | | |
| HN-1 | TSPLNIHNGQKL | SEQ ID |
| (phage | | NO.: 14 |
| display) | | |

Exemplary auxiliary moieties which include TAT peptides that can be conjugated to any of the nucleotide constructs described herein are provided in Table 2.

TABLE 2
Sequence (N′ to C′)

PEG-(PTD)
GG-(PTD)-PEG-(PTD)
PEG-(PTD)-PEG-(PTD)
GG-(PTD)-PEG-PEG-PEG-(PTD)
PEG-(PTD)-PEG-PEG-PEG-(PTD)
GG-(PTD)-PEG-(PTD)-PEG-(PTD)
GG-(PTD)-PEG-PEG-PEG-(PTD)-PEG-PEG-PEG-(PTD)
PEG = a poly(ethyleneglycol) linker having six repeat units

In a particular embodiment, the auxiliary moieties described in Table 2 include a covalent bond to Z′ at the N′ terminus, where Z′ is the residue of conjugation of 6-hydrazinonicotinic acid (HyNic) or an amino group of a polypeptide R^{Z} to an aldehyde.

Further exemplary cationic PTD (CPP) sequences are provided in Table 3.

Thus, PTDs that can be conjugated to a nucleotide construct of the invention include, but are not limited to, AntHD, TAT, VP22, cationic prion protein domains, and functional fragments thereof. Not only can these peptides pass through the plasma membrane, but the attachment of other peptide or polypeptides, such as the enzyme β-galactosidase, are sufficient to stimulate the cellular uptake of these complexes. Such chimeric proteins are present in a biologically active form within the cytoplasm and nucleus. Characterization of this process has shown that the uptake of these fusion polypeptides is rapid, often occurring within minutes, in a receptor independent fashion. Moreover, the transduction of these proteins does not appear to be affected by cell type, and these proteins can efficiently transduce ˜100% of cells in culture with no apparent toxicity (Nagahara et al., Nat. Med. 4:1449-52, 1998). In addition to full-length proteins, peptide transduction domains have also been used successfully to induce the intracellular uptake of DNA (Abu-Amer, supra), antisense polynucleotides (Astriab-Fisher et al., Pharm. Res, 19:744-54, 2002), small molecules (Polyakov et al., Bioconjug. Chem. 11:762-71, 2000) and even inorganic 40 nm iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug., Chem. 10:186-91, 1999) suggesting that there is considerable flexibility in particle size in this process.

In a particular embodiment, the invention therefore provides methods and compositions that combine the use of PTDs, such as TAT and poly-Arg, with a nucleotide construct disclosed herein to facilitate the targeted uptake of the construct into and/or release within targeted cells. Nucleotide constructs disclosed herein therefore provide methods whereby a therapeutic or diagnostic agent which is linked as an auxiliary moiety can be targeted to be delivered in certain cells by the nucleotide constructs further including one or more PTDs linked as auxiliary moieties.

The nucleotide construct of the invention can be an siRNA or other inhibitory nucleic acid sequence that itself provides a therapeutic or diagnostic benefit. However, in some instances it may be desirable to attach additional auxiliary moieties as therapeutics or to promote uptake. In the case of PTDs, the PTDs serve as additional charge modifying moieties to promote uptake of the nucleotide construct by neutralizing the charge on the nucleotide construct or typically providing a slight net cationic charge to the nucleotide construct. It will be further understood, that the nucleotide construct may include other auxiliary moieties such as, but not limited to, targeting moieties, biologically active molecules, therapeutics, small molecules (e.g., cytotoxics), and the like. In such instances the nucleotide construct having such auxillary moieties may be neutrally charged or cationically charged depending upon the auxillary moieties size and charge. In instances where the auxillary moieties are anionically charged the addition of cationically charged peptides (e.g., PTDs) can further neutralize the charge or improve the net cationic charge of the construct.

In general, the delivery domain that is linked to a nucleotide construct disclosed herein can be nearly any synthetic or naturally-occurring amino acid sequence that assists in the intracellular delivery of a nucleic construct disclosed herein into targeted cells. For example, transfection can be achieved in accordance with the invention by use of a peptide transduction domain, such as an HIV TAT protein or fragment thereof, that is covalently linked to a conjugating moiety of a nucleotide construct of the invention. Alternatively, the peptide transduction domain can include the Antennapedia homeodomain or the HSV VP22 sequence, the N-terminal fragment of a prion protein or suitable transducing fragments thereof such as those known in the art.

The type and size of the PTD will be guided by several parameters including the extent of transfection desired. Typically the PTD will be capable of transfecting at least about 20%, 25%, 50%, 75%, 80% or 90%, 95%, 98% and up to, and including, about 100% of the cells. Transfection efficiency, typically expressed as the percentage of transfected cells, can be determined by several conventional methods.

PTDs will manifest cell entry and exit rates (sometimes referred to as k₁ and k₂, respectively) that favor at least picomolar amounts of a nucleotide construct disclosed herein into a targeted cell. The entry and exit rates of the PTD and any cargo can be readily determined or at least approximated by standard kinetic analysis using detectably-labeled fusion molecules. Typically, the ratio of the entry rate to the exit rate will be in the range of between about 5 to about 100 up to about 1000.

In one embodiment, a PTD useful in the methods and compositions of the invention includes a polypeptide featuring substantial alpha-helicity. It has been discovered that transfection is optimized when the PTD exhibits significant alpha-helicity. In another embodiment, the PTD includes a sequence containing basic amino acid residues that are substantially aligned along at least one face of the peptide or polypeptide. A PTD domain useful in the invention may be a naturally occurring peptide or polypeptide or a synthetic peptide or polypeptide.

In another embodiment, the PTD includes an amino acid sequence including a strong alpha helical structure with arginine (Arg) residues down the helical cylinder.

In yet another embodiment, the PTD domain includes a polypeptide represented by the following general formula: B_{P1}-X_{P1}-X_{P2}-X_{P3}-B_{P2}-X_{P4}-X_{P5}-B_{P3} where B_{P1}, B_{P2}, and B_{P3} are each independently a basic amino acid, the same or different; and X_{P1}, X_{P2}, X_{P3}, X_{P4}, and X_{P5} are each independently an alpha-helix enhancing amino acid, the same or different.

In another embodiment, the PTD domain is represented by the following general formula: B_{P1}-X_{P1}-X_{P2}-B_{P2}-B_{P3}-X_{P3}-X_{P4}-B_{P4} where B_{P1}, B_{P2}, B_{P3}, and B_{P4} are each independently a basic amino acid, the same or different; and X_{P1}, X_{P2}, X_{P3}, and X_{P4} are each independently an alpha-helix enhancing amino acid the same or different.

Additionally, PTD domains include basic residues, e.g., lysine (Lys) or arginine (Arg), and further can include at least one proline (Pro) residue sufficient to introduce “kinks” into the domain. Examples of such domains include the transduction domains of prions. For example, such a polypeptide contains KKRPKPG (SEQ ID NO:15).

In one embodiment, the domain is a polypeptide represented by the following sequence: X_{P}—X_{P}-R-X_{P}-(P/X_{P})-(B_{P}/X_{P})-B_{P}-(P/X_{P})-X_{P}-B_{P}-(B_{P}/X_{P}), where X is any alpha helical promoting residue such as alanine; P/X_{P} is either proline or X_{P} as previously defined; B_{P} is a basic amino acid residue, e.g., arginine (Arg) or lysine (Lys); R is arginine (Arg) and B_{P}/X_{P} is either B_{P} or X_{P} as defined above.

In another embodiment the PTD is cationic and consists of between 7 and 10 amino acids and has the formula KX_{P1}RX_{P2}X_{P1}, where X_{P1} is R or K and X_{P2} is any amino acid. An example of such a polypeptide contains RKKRRQRRR (SEQ ID NO:1). In another example, the PTD is a cationic peptide sequence having 5-10 arginine (and/or lysine) residues over 5-15 amino acids.

Additional delivery domains in accord with this disclosure include a TAT fragment that contains at least amino acids 49 to 56 of TAT (SEQ ID NO:1) up to about the full-length TAT sequence (see, e.g., SEQ ID NO:16). A TAT fragment may include one or more amino acid changes sufficient to increase the alpha-helicity of the fragment. In some instances, the amino acid changes introduced will involve adding a recognized alpha-helix enhancing amino acid. Alternatively, the amino acid changes will involve removing one or more amino acids from the TAT fragment that impede alpha helix formation or stability. In a more specific embodiment, the TAT fragment will include at least one amino acid substitution with an alpha-helix enhancing amino acid. Typically the TAT fragment will be made by standard peptide synthesis techniques although recombinant DNA approaches may be used in some cases. In one embodiment, the substitution is selected so that at least two basic amino acid residues in the TAT fragment are substantially aligned along at least one face of that TAT fragment. In a more specific embodiment, the substitution is chosen so that at least two basic amino acid residues in the TAT 49-56 sequence (SEQ ID NO:1) are substantially aligned along at least one face of that sequence.

Additional transduction proteins (PTDs) that can be used in the compositions and methods of the invention include the TAT fragment in which the TAT 49-56 sequence has been modified so that at least two basic amino acids in the sequence are substantially aligned along at least one face of the TAT fragment. Illustrative TAT fragments include at least one specified amino acid substitution in at least amino acids 49-56 of TAT which substitution aligns the basic amino acid residues of the 49-56 sequence along at least one face of the segment and typically the TAT 49-56 sequence.

Also included are chimeric PTD domains. Such chimeric PTDs include parts of at least two different transducing proteins. For example, chimeric PTDs can be formed by fusing two different TAT fragments, e.g., one from HIV-1 (SEQ ID NO:16) and the other from HIV-2 (SEQ ID NO:17) or one from a prion protein (SEQ ID NO:18) and one from HIV.

A PTD can be linked as an auxiliary moiety to a nucleotide construct of the invention using phosphoramidate or phosphotriester linkers at an internucleotide bridging group or at the 3′ or 5′ ends. For example, a siRNA construct containing a 3′-amino group with a 3-carbon linker may be utilized for linking the siRNA construct to a PTD. The siRNA construct may be conjugated to the PTD via a heterobifunctional cross linker.

The PTD can be attached as anauxiliary moiety to anucleotide construct via a bioreversible group, whereby the bioreversible group can be cleaved intracellularly, e.g., by an intracellular enzyme (e.g., protein disulfide isomerase, thioredoxin, or a thioesterase) and thereby release the polynucleotide.

For example, in addition to the PTD being conjugated between the 5and 3ends, a PTD can be conjugated directly to a polynucleotide (e.g., an RNA or DNA) containing anucleotide construct disclosed herein, at the 5′ and/or 3′end via a free thiol group. For example, a PTD can be linked to the polynucleotide by a disulfide linkage. This approach can be applied to any polynucleotide length and will allow for delivery of the polynucleotide (e.g., siRNA) into cells. The polynucleotide can also include, for example, one or more delivery domains and/or a protecting group that contains a basic group. Once inside the cell the polynucleotide reverts to an unprotected polynucleotide based on the intracellular conditions, e.g., reducing environment, by hydrolysis or other enzymatic activity (e.g., protein disulfide isomerase, thioredoxin, or thioesterase activity).

TABLE 3

| Compound | SEQ ID | | C- | MW | MW |
|---|---|---|---|---|---|
| # | NO: | Structure | Terminus | Calcd | Observ |
| P01 | 19 | HyNic GGRK′RK′RK′RK′RK′RK′RK′R | CONH₂ | 2412 | 2413 |
| P02 | 20 | HyNic GGRK′RK′RK′RK′RK′RK′RK′RK′RK′RK′RK′R | CONH₂ | 3548 | 3547 |
| P03 | 21 | HyNic GGRK′RK′RK′RK′RK′RK′RK′RK′RK′RK′RK′RK′RK′RK′R | CONH₂ | 4665 | 4668 |
| | | KR | | | |
| P04 | 22 | HyNic GGESDSELEIKRYKNRVASRKSRAKFKOLLQHYREVAA | CONH₂ | 6557 | 6563 |
| | | AKSSENDRLRLLLKQSS | | | |
| P05 | 23 | HyNic GGSRRHHSRSKAKRSRHH | CONH₂ | 2312 | 2311 |
| P06 | 24 | HyNic GGAYDLRRRERQSRLRRRERQSR | CONH₂ | 3134 | 3132 |
| P07 | 25 | HyNic GGMAPQRDTVGGRTTPPSWGPAKAQLRNSCA | CONH₂ | 3344 | 3342 |
| P08 | 26 | HyNic GGMAPQRDTVGGRTTPPSWGPAKAQLRNSSA | CONH₂ | 3328 | 3327 |
| P09 | 27 | HyNic GGFCIGRL | CONH₂ | 997 | 997 |
| P10 | 28 | HyNic GGGVIGRL | CONH₂ | 994 | 993 |
| P11 | 29 | HyNic GGRAWMRWYSPTTRRYG | CONH₂ | 2277 | 2276 |
| P12 | 30 | HyNic GGPLILLRLLR | CONH₂ | 1396 | 1395 |
| P13 | 31 | HyNic GGMIIYRDLISH | CONH₂ | 1533 | 1532 |
| P14 | 32 | HyNic GGACTGSTQHQCG | CONH₂ | 1380 | 1378 |
| P15 | 33 | HyNic GGALFLGWLGAAGSTMGAPKSKRKV | CONH₂ | 2619 | 2618 |
| P16 | 34 | HyNic GGLIRLWSHLIHIWFQNRRLKWKKK | CONH₂ | 3214 | 3211 |
| P17 | 35 | HyNic GGIGAVLKVLTTGLPALISWIKRKRQQ | CONH₂ | 3081 | 3079 |
| P18 | 36 | HyNic GGLHKLLHHLLHHLHKLLHHLHHLLHKL | CONH₂ | 3559 | 3556 |
| P19 | 37 | HyNic GGRKKR | CONH₂ | 875 | 875 |
| P20 | 38 | HyNic GGRKKRRQRRR | CONH₂ | 1629 | 1627 |
| P21 | 39 | HyNic GGRKKRRQRRRGGRKKR | CONH₂ | 2311 | 2309 |
| P22 | 40 | N₃ GGRKKRRQRRR-Peg24-GGRKKRRQRRR-Peg24- | CONH₂ | 6459 | 6450 |
| | | GGRKKRRQRRR | | | |
| P23 | 41 | HyNic GGRKKRRQRRR-Peg24-GGRKKRRQRRR-Peg24- | CONH₂ | 6379 | 6385 |
| | | GGRKKRRQRRR | | | |
| P24 | 42 | HyNic GGRK′RK′RK′RK′RK′RK′RK′RK′RK′RK′RK′RC(Peg24) | CONH₂ | 4928 | 4934 |
| P25 | 43 | HyNic GGRK′RK′RK′RK′RK′RK′RK′RK′RK′RK′RK′RC(Peg48) | CONH₂ | 5980 | 5987 |
| P26 | 44 | HyNic GGRKKRRQRRR-Peg24-GGRKKRRQRRR-Peg24- | CONH₂ | 6754 | 6777 |
| | | GGRKKRRQRRRK(Hexanoic Acid) | | | |
| P27 | 45 | GGLHKLLHHLLHHLHKLLHHLHHLLHKL | CONH₂ | 3382 | 3380 |
| P28 | 46 | GGACTGSTQHQCG | CONH₂ | 1205 | 1203 |
| P29 | 47 | GGLIRLWSHLIHIWFQNRRLKWKKK | CONH₂ | 3214 | 3211 |
| P30 | 48 | GGALFLGWLGAAGSTMGAPKSKRKV | CONH₂ | 2444 | 2442 |
| P31 | 49 | GGIGAVLKVLTTGLPALISWIKRKRQQ | CONH₂ | 2904 | 2903 |
| P32 | 50 | HyNic GGLFGAIAGFIENGWEGMIDGWYG | CONH₂ | 2693 | 2695 |
| P33 | 51 | HyNic GGLFEAIEGFIENGWEGMIDGWYG | CONH₂ | 2821 | 2844 |
| P34 | 52 | HyNic GGLFEAIEGFIENGWEGMIDGWYGRKKRRQRRR | CONH₂ | 4144 | 4142 |
| P35 | 53 | HyNic GGLFEAIEGFIENGWEGLIEGWYG | CONH₂ | 2833 | 2856 |
| P36 | 54 | HyNic GGKWKLFKKIGAVLKVLTTGYGRKKRRQRRR | CONH₂ | 3862 | 3861 |
| P37 | 55 | AzidePEG4-ILSSLTVTQLLRRLHQWI | CONH₂ | 2449 | 2449 |
| P38 | 56 | AzidePEG4-MKWVTFISLLFLFFSSAYS | CONH₂ | 2413 | 2411 |
| P39 | 57 | AzidePEG4-MIRTLLLSTLVAGALS | CONH₂ | 1932 | 1931 |
| P40 | 58 | AzidePEG4-RLIEDICLPRWGCLWEDD | CONH₂ | 2503 | 2502 |
| P41 | | Azide-C18 | — | 267 | 267 |
| P42 | 59 | AzidePEG4-KDEL | CONH₂ | 777 | 776 |
| P43 | 60 | AzidePEG4-LFEAIEGFIENGWEGMIDGWYGKDEL | CONH₂ | 3291 | |
| P44 | 61 | AzidePEG4-LFEAIEGFIENGWEGMIDGWYGRKKRRQRRRKDEL | CONH₂ | 4614 | |
| P45 | 62 | Azide-PEG4 RLIEDICLPRWGCLWEDD (Albumin binding) | CONH₂ | 2503 | 2502 |
| P46 | 63 | Azide-PEG4 MKLSLVAAMLLLLSAARA (ER targeting) | CONH₂ | 2145 | 2144 |
| P47 | 64 | Azide-PEG4 MKLAVTLTLVTLALSSSSASA (ER targeting) | CONH₂ | 2332 | 2348 |
| P48 | 65 | Azide-PEG4 FFKKLAHALHLLALLALHLAHALKKA (Endosomolytic) | CONH₂ | 3161 | 3161 |
| P49 | 66 | Azide-PEG4 PSQPTYPGDDAPVRDLIRFYRDLRRYLNVVTRHRY | CONH₂ | 4578 | 4579 |
| P50 | 67 | Azide-PEG4 RLIEDICLPRWGCLWEDDKDEL (ER targeting) | CONH₂ | 2988 | 2987 |
| P51 | 68 | Azide-PEG4 LFEAIEGFIENGWGMIDGWYG (Endosomolytic) | CONH₂ | 2804 | 2802 |
| P52 | 69 | Azide-PEG4 LFEAIEGFIENGWEGMIDGWYGRKKRRQRRR | CONH₂ | 4127 | 4127 |
| | | (Endosomolytic) | | | |
| P53 | 70 | Azide-PEG4 MIRTLLLSTLVAGALSKDEL (ER targeting) | CONH₂ | 2417 | 2416 |
| P54 | 71 | Ac YEQDPWGVKWWYK(Peg4-N3) | CONH₂ | 2100 | 2099 |
| P55 | 72 | NH2 MIRTLLLSTLVAGALSK(Peg4-N3) (ER targeting) | CONH₂ | 2057 | 2059 |
| P56 | 73 | NH2 YEQDPWGVKWWYK(Peg4-N3) | CONH₂ | 2058 | 2057 |
| P57 | 74 | Azide-PEG4 R-Bip-R-Bip-R (Albumin binding) | CONH₂ | 1205 | 1205 |
| P58 | 75 | Azide-PEG4 R-Bip-R (Albumin binding) | CONH₂ | 826 | 827 |
| P59 | 76 | NH2 ILSSLTVTQLLRRLHQWIK(Peg4-N3) (ER targeting) | CONH₂ | 2577 | 2579 |
| P60 | 77 | NH2 MIRTLLLSTLVAGALSKDEL(Peg4-N3) (ER targeting) | CONH₂ | 2544 | 2544 |
| P61 | 78 | Azide-PEG4 LFEAIEGFIENGWEGMIDGWYGRKKRRQRRRKDEL | CONH₂ | 4610 | 4609 |
| P62 | 79 | Azide-PEG4 IGAVLKVLTTGLPALISWIKRKRQQ (Endosomolytic) | CONH₂ | 3062 | 3061 |
| P63 | 80 | Azide-PEG4 IGAVLKVLTTGLPALISWIKRKRQQKDEL | CONH₂ | 3550 | 3548 |
| P64 | 81 | HyNic-GGGPRRRRSSRRP (endosomolytic) | CONH₂ | 1670 | 1668 |
| P65 | 82 | HyNic-GGGVRRRRRPRVS (endosomolytic) | CONH₂ | 1684 | 1683 |
| P66 | 83 | HyNic-GGGPRRRRSSRRPVRRRRRPRVS (endosomolytic) | CONH₂ | 2991 | 2989 |
| P67 | 84 | HyNic-GGGPRRRRSSRRPVRRRRRPRVSRRRRRRGGRRRR | CONH₂ | 4666 | 4666 |
| | | (endosomolytic) | | | |
| P68 | 85 | HyNic-GGSRRHHSRSKAKRSRHH (endosomolytic) | CONH₂ | 2314 | 2312 |
| P69 | 86 | HyNic-RRRRRRRRR (endosomolytic) | CONH₂ | 1600 | 1599 |
| P70 | 87 | HyNic-GGWEAALAEALAEALAEHLAEALAEALEALAA | CONH₂ | 3323 | 3321 |
| | | (endosomolytic) | | | |
| P71 | 88 | HyNic-GGWEAKLAKALAKALAKHLAKALAKALAKALLA | CONH2 | 3417 | 3416 |
| | | (endosomolytic) | | | |
| P72 | 89 | c(RGDfK(N₃-Peg8)) (targeting peptide) | N/A | 1053 | 1052 |
| P73 | 90 | N₃-Peg8-E(c(RGDfK))-E(c(RGDfK)₂) (targeting peptide) | N/A | 2482 | 2463 |
| P74 | 91 | N₃-Peg8-c(CRGDRGPDC) (targeting peptide) | CONH₂ | 1426 | 1424 |
| P75 | 92 | N₃-Peg8-c(CRGDKGPDC) (targeting peptide) | CONH₂ | 1399 | 1396 |
| P76 | 93 | N₃-Peg8-c(CRNDRGPDC) (targeting peptide) | CONH₂ | 1425 | 1423 |
| P 77 | 94 | N₃-Peg8-YTIWMPENPRPGTPCDIFTNSRGKRASNG (targeting | CONH₂ | 3714 | 3712 |
| | | peptide) | | | |
| P78 | 95 | N₃-Peg8-YTSLIHSLIEESQNQQEKQEKELMELERWGSMLKC | CONH₂ | 4689 | 4688 |
| | | (targeting peptide) | | | |
| P79 | 96 | N₃-Peg8- YTSLIHSLIEESQNQQEKQEKELMELERWGSMLQL | CONH₂ | 4689 | 4689 |
| | | (targeting peptide) | | | |
| P80 | 97 | HyNic-GGPSQPTYPGDDAPVRDLIRFYRDLRRYLNVVTRHRY | CONH₂ | 4598 | 4597 |
| | | (endosomolytic) | | | |
| P81 | 98 | N3-Peg4-AAKDEL | COOH | 919.0 | 919 |
| P82 | 99 | N3-Peg4-GEEDTSEKDEL | COOH | 1524.5 | 770.5 |
| | | | | | (m/2z) |
| P83 | 100 | N3-Peg4-ASQPGKPPKDEL | COOH | 1539.7 | 761 |
| | | | | | (m/2z) |
| P84 | 101 | N3-Peg4-ASQPGKPPREDL | COOH | 1567.7 | |
| P85 | 102 | N3-Peg4-GRQSDIDTHNRIKDEL | COOH | 2170.3 | |

In Table 3: (1) HyNic=hydrazine-nicotinamide, K′=Boc-Lys(Fmoc)-OH; Bip: Bis-phenylalanine; (2) compounds P01, P02, P03, P04, P05, P06, P07, P08, P09, P10, P11, P12, P13, P14, P15, P16, P19, P20, P21, P22, P23, P24, P25, and P26include cell-penetrating peptides; compounds P16, P17, P18, P27, P28, P29, P31, P32, P33, P34, P35, and P36 include endosomolytic peptides; compounds P37, P38, and P39 include peptides targeting the endoplasmic reticulum; compounds P40 and P41 include albumin-binding moieties, and compound P42 includes a KDEL receptor targeting moiety. Other compounds are as noted in the table.

Peptide linkers that can be used in the constructs and methods of the invention will typically include up to about 20 or 30 amino acids, commonly up to about 10 or 15 amino acids, and still more often from about 1 to 5 amino acids. The linker sequence is generally flexible so as not to hold the fusion molecule in a single rigid conformation. The linker sequence can be used, e.g., to space the PTD domain from the nucleic acid. For example, the peptide linker sequence can be positioned between the peptide transduction domain and the nucleic acid domain, e.g., to provide molecular flexibility. The length of the linker moiety is chosen to optimize the biological activity of the peptide or polypeptide including, for example, a PTD domain fusion construct and can be determined empirically without undue experimentation. Examples of linker moieties are -Gly-Gly-, GGGGS (SEQ ID NO:106), (GGGGS)_{N}, GKSSGSGSESKS (SEQ ID NO:107), GSTSGSGKSSEGKG (SEQ ID NO:108), GSTSGSGKSSEGSGSTKG (SEQ ID NO:109), GSTSGSGKPGSGEGSTKG (SEQ ID NO:110), or EGKSSGSGSESKEF (SEQ ID NO:111). Peptide or polypeptide linking moieties are described, for example, in Huston et al., Proc. Nat'l Acad. Sci. 85:5879, 1988; Whitlow et al., Protein Engineering 6:989, 1993; and Newton et al., Biochemistry 35:545, 1996. Other suitable peptide or polypeptide linkers are those described in U.S. Pat. Nos. 4,751,180 and 4,935,233, which are hereby incorporated by reference.

### Pharmaceutical Compositions

Delivery of a nucleotide construct of the invention can be achieved by contacting a cell with the construct using a variety of methods known to those of skill in the art. In particular embodiments, a nucleotide construct of the invention is formulated with various carriers, dispersion agents and the like, as are described more fully elsewhere herein.

A pharmaceutical composition according to the invention can be prepared to include a nucleotide construct disclosed herein, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington: The Science and Practice of Pharmacy, 21^{st} Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions according to the invention may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, transdermal application, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a pharmaceutically acceptable carrier is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the invention are related to the characteristics of the pharmaceutical composition and the particular therapeutic effect to be achieve. The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the the ingredients.

For topical formulations, the base composition can be prepared with any solvent system, such as those Generally Regarded as Safe (GRAS) by the U.S. Food & Drug Administration (FDA). GRAS solvent systems include many short chain hydrocarbons, such as butane, propane, n-butane, or a mixture thereof, as the delivery vehicle, which are approved by the FDA for topical use. The topical compositions can be formulated using any dermatologically acceptable carrier. Exemplary carriers include a solid carrier, such as alumina, clay, microcrystalline cellulose, silica, or talc; and/or a liquid carrier, such as an alcohol, a glycol, or a water-alcohol/glycol blend. The compounds may also be administered in liposomal formulations that allow compounds to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. Nos. 4,877,805, 4,980,378, 5,082,866, 6,118,020 and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

Topical compositions can be provided in any useful form. For example, the compositions of the invention may be formulated as solutions, emulsions (including microemulsions), suspensions, creams, foams, lotions, gels, powders, balm, or other typical solid, semi-solid, or liquid compositions used for application to the skin or other tissues where the compositions may be used. Such compositions may contain other ingredients typically used in such products, such as colorants, fragrances, thickeners, antimicrobials, solvents, surfactants, detergents, gelling agents, antioxidants, fillers, dyestuffs, viscosity-controlling agents, preservatives, humectants, emollients (e.g., natural or synthetic oils, hydrocarbon oils, waxes, or silicones), hydration agents, chelating agents, demulcents, solubilizing excipients, adjuvants, dispersants, skin penetration enhancers, plasticizing agents, preservatives, stabilizers, demulsifiers, wetting agents, sunscreens, emulsifiers, moisturizers, astringents, deodorants, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomedicinals.

In some formulations, the composition is formulated for ocular application. For example, a pharmaceutical formulation for ocular application can include a polynucleotide construct as described herein in an amount that is, e.g., up to 99% by weight mixed with a physiologically acceptable ophthalmic carrier medium such as water, buffer, saline, glycine, hyaluronic acid, mannitol, and the like. For ophthalmic delivery, a polynucleotide construct as described herein may be combined with ophthalmologically acceptable preservatives, co-solvents, surfactants, viscosity enhancers, penetration enhancers, buffers, sodium chloride, or water to form an aqueous, sterile ophthalmic suspension or solution. Ophthalmic solution formulations may be prepared by dissolving the polynucleotide construct in a physiologically acceptable isotonic aqueous buffer. Further, the ophthalmic solution may include an ophthalmologically acceptable surfactant to assist in dissolving the inhibitor. Viscosity building agents, such as hydroxymethyl cellulose, hydroxyethyl cellulose, methylcellulose, polyvinylpyrrolidone, or the like may be added to the compositions of the invention to improve the retention of the compound.

Topical compositions can be delivered to the surface of the eye, e.g., one to four times per day, or on an extended delivery schedule such as daily, weekly, bi-weekly, monthly, or longer, according to the routine discretion of a skilled clinician. The pH of the formulation can range from about pH 4-9, or about pH 4.5 to pH 7.4.

For nucleotide constructs of the invention, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

While the nucleotide constructs described herein may not require the use of a carrier for delivery to the target cell, the use of carriers may be advantageous in some embodiments. Thus, for delivery to the target cell, the nucleotide construct of the invention can non-covalently bind a carrier to form a complex. The carrier can be used to alter biodistribution after delivery, to enhance uptake, to increase half-life or stability of the polynucleotide (e.g., improve nuclease resistance), and/or to increase targeting to a particular cell or tissue type.

Exemplary carriers include a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

Other therapeutic agents as described herein may be included in a pharmaceutical composition of the invention in combination with a nucleotide construct of the invention.

### Intracellular Activity of Nucleotide Constructs

The invention provides compositions and methods for delivering nucleotide constructs disclosed herein (e.g., RNA, DNA, nucleic acids including modified bases, other anionic nucleic acids, and the like). The invention therefore provides methods and compositions useful for delivery of non-coding nucleotide constructs that exert a regulating effect on gene or protein expression.

Polynucleotide constructs of the invention may be single stranded or double stranded. When double stranded, one or both strands may include one or more bioreversible groups. When the polynucleotide acts as siRNA, the passenger strand may include a group that is irreversibly bound to an internucleotide bridging group, e.g., a C₁₋₆ alkyl phosphotriester. Typically, such a group is located after the first or second nucleotide from the 3′ end. The irreversible group prevents the passenger strand from acting as a guide strand and thereby prevents or reduces possible off-target effects.

RNA interference (RNAi) is the process whereby messenger RNA (mRNA) is degraded by small interfering RNA (siRNA) derived from double-stranded RNA (dsRNA) containing an identical or very similar nucleotide sequence to that of a target gene to be silenced. This process prevents the production of a protein encoded by the targeted gene through post-transcriptional, pre-translational manipulation. Accordingly, silencing of dominant disease genes or other target genes can be accomplished.

In vivo RNAi proceeds by a process in which the dsRNA is cleaved into short interfering RNAs (siRNAs) by an enzyme called Dicer, a dsRNA endoribonuclease, (Bernstein et al., 2001; Hamilton & Baulcombe, 1999, Science 286: 950; Meister and Tuschl, 2004, Nature 431, 343-9), thus producing multiple molecules from the original single dsRNA. siRNAs are loaded into the multimeric RNAi Silencing Complex (RISC) resulting in both catalytic activation and mRNA target specificity (Hannon and Rossi, Nature 431, 371-378, 2004; Novina and Sharp, Nature 430, 161-164, 2004). During siRNA loading into RISC, the antisense or guide strand is separated from the siRNA and remains docked in Argonaute-2 (Ago2), the RISC catalytic subunit (Leuschner et al., EMBO Rep. 7, 314-320, 2006). Certain cellular compartments, such as endoplasmic reticulum (ER), Golgi apparatus, ER-Golgi intermediate compartment (ERGIC), P-bodies, and early endosomes are enriched in Ago2. mRNAs exported from the nucleus into the cytoplasm are thought to pass through activated RISCs prior to ribosomal arrival, thereby allowing for directed, post-transcriptional, pre-translational regulation of gene expression. In theory, each and every cellular mRNA can be regulated by induction of a selective RNAi response.

The ability of 21-23 bp siRNAs to efficiently induce an RNAi response in mammalian cells is now routine (Sontheimer, Nat. Rev. Mol. Cell. Biol. 6, 127-138, 2005). The IC₅₀ for siRNAs is in the 10-100 pM range, significantly below the best drugs with IC₅₀ values in the 1-10 nM range. Consequently, due to its exquisite selectivity, RNAi has become a corner-stone for directed manipulation of cellular phenotypes, mapping genetic pathways, discovering and validating therapeutic targets, and has significant therapeutic potential.

Aspects of RNAi include (1) dsRNA, rather than single-stranded antisense RNA, is the interfering agent; (2) the process is highly specific and is remarkably potent (only a few dsRNA molecules per cell are required for effective interference); (3) the interfering activity (and presumably the dsRNA) can cause interference in cells and tissues far removed from the site of introduction. However, effective delivery of dsRNA is difficult. For example, a 21 bp dsRNA with a molecular weight of 13,860 Daltons cannot traverse the cell membrane to enter the cytoplasm, due to (1) the size and (2) the extremely negative (acidic) charge of the RNA. The methods and compositions provided by the invention enable the delivery of nucleotide constructs, such as dsRNA, into a cell through charge neutralization and improved uptake.

dsRNA including siRNA sequences that are complementary to a nucleotide sequence of the target gene can be prepared in any number of methods. Methods and techniques for identifying siRNA sequences are known in the art. The siRNA nucleotide sequence can be obtained from the siRNA Selection Program, Whitehead Institute for Biomedical Research, Massachusetts Institute of Technology, Cambridge, Mass. (currently available at http:[//]jura.wi.mit.edu/bioc/siRNAext/; note that brackets have been added to remove hyperlinks) after supplying the Accession Number or GI number from the National Center for Biotechnology Information website (available on the World Wide Web at ncbi.nlm.nih.gov). Alternatively, dsRNA containing appropriate siRNA sequences can be ascertained using the strategy of Miyagishi and Taira (2003). Commercially available RNAi designer algorithms also exist (http:[//]rnaidesigner.invitrogen.com/rnaiexpress). Preparation of RNA to order is commercially available.

Nucleotide constructs of the invention may also act as miRNA to induce cleavage of mRNA. Alternatively, nucleotide constructs of the invention may act as antisense agents to bind to mRNA, either to induce cleavage by RNase or to sterically block translation.

Exemplary methods by which the nucleotide constructs of the invention can be transported into a cell are described herein.

### Therapeutic Methods

Various diseases and disorders can be treated using nucleotide constructs of the invention. For example, growth of tumor cells can be inhibited, suppressed, or destroyed upon delivery of an anti-tumor siRNA. For example, an anti-tumor siRNA can be an siRNA targeted to a gene encoding a polypeptide that promotes angiogenesis. Various angiogenic proteins associated with tumor growth are known in the art. The nucleotide constructs described herein can therefore be used in the treatment of diseases such as anti-proliferative disorders (e.g., cancer), virus infections, and genetic diseases. Other diseases that may be treated using polynucleotides on the invention are in ocular disorders such as age-related macular degeneration (e.g., as described in U.S. Pat. No. 7,879,813 and U.S. 2009/0012030) and topical disorders such as psoriasis.

The compositions containing an effective amount can be administered for prophylactic or therapeutic treatments. In prophylactic applications, compositions can be administered to a subject with a clinically determined predisposition or increased susceptibility to cancer, or any disease described herein. Compositions of the invention can be administered to the subject (e.g., a human) in an amount sufficient to delay, reduce, or prevent the onset of clinical disease. In therapeutic applications, compositions are administered to a subject (e.g., a human) already suffering from disease (e.g., cancer, such as leukemia or a myelodysplastic syndrome) in an amount sufficient to cure or at least partially arrest the symptoms of the condition and its complications.

Amounts effective for this use may depend on the severity of the disease or condition and the weight and general state of the subject, but generally range from about 0.05 μg to about 1000 μg (e.g., 0.5-100 μg) of an equivalent amount of the agent per dose per subject. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6 hours, 8-12 hours 14-16 hours, 18-24 hours, every 2-4 days, every 1-2 weeks, and once a month). Alternatively, continuous intravenous infusions sufficient to maintain therapeutically effective concentrations in the blood are contemplated.

The therapeutically effective amount of one or more agents present within the compositions of the invention and used in the methods of this disclosure applied to mammals (e.g., humans) can be determined by the ordinarily-skilled artisan with consideration of individual differences in age, weight, and the condition of the mammal. Single or multiple administrations of the compositions of the invention including an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

One or more nucleotide constructs of the invention may be used in combination with either conventional methods of treatment or therapy or may be used separately from conventional methods of treatment or therapy.

When one or more nucleotide constructs of the invention are administered in combination therapies with other agents, they may be administered sequentially or concurrently to an individual. Alternatively, pharmaceutical compositions according to the invention may contain a combination of a nucleotide construct of the invention in association with a pharmaceutically acceptable excipient, as described herein, and another therapeutic or prophylactic agent known in the art.

The following examples are meant to illustrate the invention. They are not meant to limit the invention in any way.

### EXAMPLES

### Example 1. Synthesis and Purification of the Nucleotides and Polynucleotides of the Invention

### General Synthesis Procedure

The polynucleotide constructs of the invention can be prepared according to the generalized and specific methods and schemes described herein. For example, starting materials containing thiols underwent a reaction with 2,2′-dipyridyl disulfide affording the corresponding pyridyl disulfide compounds (e.g., see Scheme 1), which were then subjected to a reaction with nucleoside phosphordiamidites to generate nucleotide constructs of the invention (e.g., see Scheme 1). These nucleotide constructs were then used in standard oligonucleotide synthesis protocols to form polynucleotide constructs. These polynucleotide constructs were then deprotected and purified using HPLC.

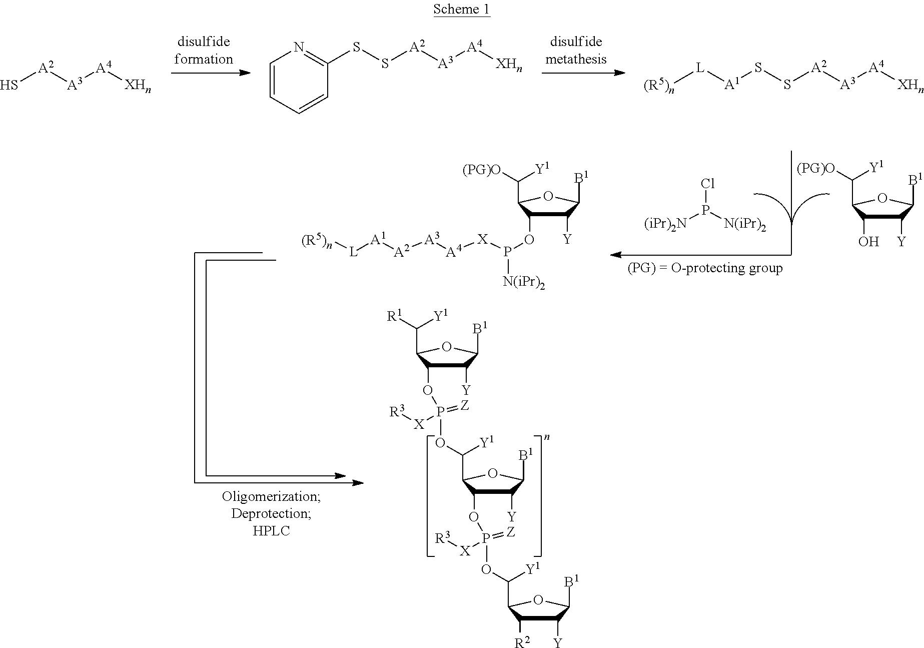

### Specific Syntheses of the Nucleotides of the Invention

Exemplary syntheses of nucleotides of the invention and precursors thereof are described below.

### Precursors

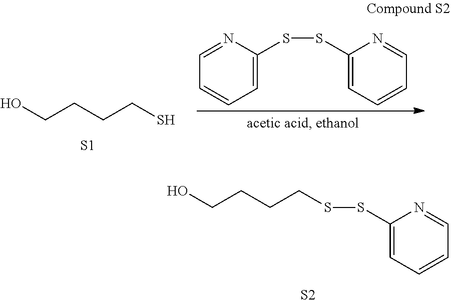

To a solution of 4-Mecaptol-butanol (10.0 g, 94 mmol) and dithiopyridine (25.0 g, 113 mmol) in 400 mL of ethanol was added 7.0 mL of acetic acid. The reaction mixture was stirred for 1 hour at room temperature before being concentrated under vacuum. 500 mL of ethyl acetate was added to the crude product and the solution was washed sequentially with aqueous 1N NaOH solution (200 mL) and brine (200 mL), and then dried over anhydrous Na₂SO₄. The solvent was evaporated in vacuo and the crude mixture was purified by silica gel column chromatography using ethyl acetate/hexane solvent system (0-40% gradient on Combi Flash Rf Instrument) to give 12.8 g (64%) of product S2 as colorless oil. ¹H NMR (500 MHz): δ8.45 (d, J 4.5 Hz, 1H), 7.70 (d, J 8.0 Hz, 1H), 7.62 (m, 1H), 7.06 (m, 1H), 3.65 (t, J 6.0 Hz, 2H), 2.83 (t, J 7.0 Hz, 2H), 1.80 (m, 2H), 1.70 (br s, 1H), 1.65 (m, 2H).

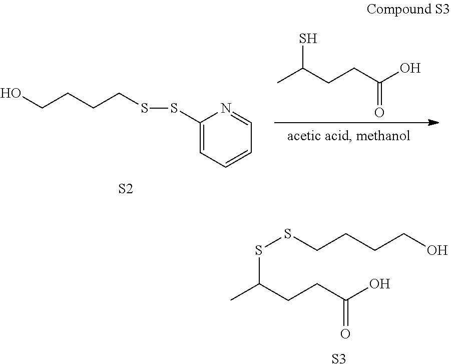

To a solution of S2 (1.3 g, 6.0 mmol) and 4-sulfanylpentanoic acid (0.67 g, 5.0 mmol) in 30 mL of methanol was added 30 μL of acetic acid. The reaction mixture was stirred for 16 hours at room temperature before being condensed in vacuo. The crude mixture was purified by silica gel column chromatography using ethyl acetate/hexane/2% acetic acid solvent system (0-70% gradient on Combi Flash Rf Instrument) to give 1.13 g (95%) of product S3 as colorless oil. ¹H NMR (500 MHz): δ4.95 (br s, 1H), 3.68 (t, J 6.0 Hz, 2H), 2.88 (m, 1H), 2.71 (t, J 7.0 Hz, 2H), 2.50 (m, 2H), 1.98 (m, 1H), 1.18 (m, 1H), 1.75 (m, 2H), 1.65 (m, 2H), 1.32 (d, J 7.0 Hz, 3H).

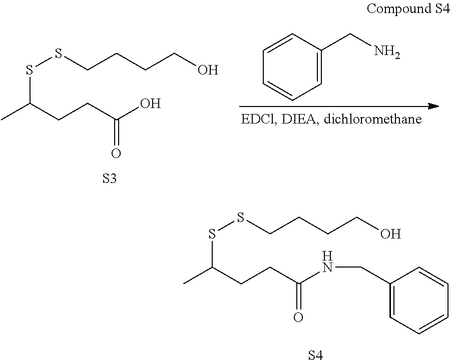 [SMILES: CC(CCC(=O)O)SSCCCCO ; NCc1ccccc1 ; "S3" ; CC(CCC(=O)NCc1ccccc1)SSCCCCO ; "S4"]

To a solution of S3 (1.13 g, 5.0 mmol), benzylamine (0.84 mL, 7.7 mmol) and 3.6 mL of N, N-diisopropylethylamine (DIEA) in 25.0 mL of dichloromethane was added 1-ethyl-3-(3-dimethylaminopropyl) carbodiimide (EDCl, 1.5 g, 7.7 mmol). The reaction mixture was stirred for 2 hours at room temperature before being concentrated in vacuo. The crude mixture was purified by silica gel column chromatography using ethyl acetate/hexane solvent system (0-100% gradient on Combi Flash Rf Instrument) to give 1.17 g (70%) of product S4 as colorless oil. ¹H NMR (500 MHz): δ7.22-7.31 (m, 5H), 6.55 (br s, 1H, 4.35 (d, J 5.5 Hz, 2H), 4.20 (br s, 1H), 3.55 (m, 2H), 2.80 (m, 1H), 2.60 (t, J 7.5 Hz, 2H), 2.25 (t, J 7.5 Hz, 2H), 1.85 (m, 1H), 1.75 (m, 1H), 1.65 (m, 2H), 1.55 (m, 2H), 1.25 (d, J 6.5 Hz, 3H).

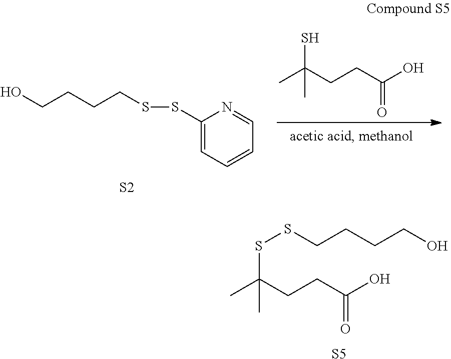

To a solution of S2 (1.82 g, 8.4 mmol) and 4-sulfanyl-4-methylpentanoic acid (1.04 g, 7.0 mmol) in 45.0 mL of methanol was added 35 μL of acetic acid. The reaction mixture was stirred for 16 hours at room temperature before being concentrated in vacuo. The crude mixture was purified by silica gel column chromatography using ethyl acetate/hexane/2% acetic acid solvent system (0-70% gradient on Combi Flash Rf Instrument) to give 0.82 g (50%) of product S5 as colorless oil. ¹H NMR (500 MHz): δ7.25 (br s, 1H), 3.63 (t, J 6.0 Hz, 2H), 2.69 (m, 2H), 2.40 (m, 2H), 1.83 (m, 2H), 1.70 (m, 2H), 1.62 (m, 2H), 1.25 (s, 6H).

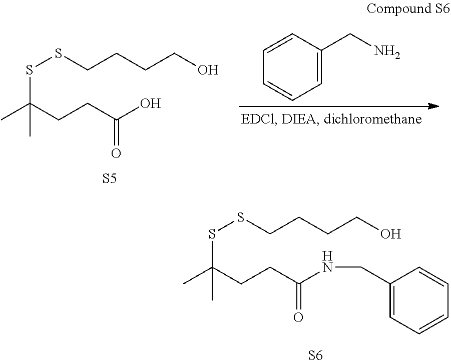

To a solution of S5 (0.82 g, 3.25 mmol), benzylamine (0.53 mL, 4.88 mmol) and 2.3 mL of N, N-diisopropylethylamine (DIEA) in 20.0 mL of dichloromethane was added 1-ethyl-3-(3-dimethylaminopropyl) carbodiimide (EDCl, 0.94 g, 4.88 mmol). The reaction mixture was stirred for 2 hours at room temperature before being concentrated in vacuo. The crude mixture was purified by silica gel column chromatography using ethyl acetate/hexane solvent system (0-100% gradient on Combi Flash Rf Instrument) to give 0.80 g (73%) of product S6 as colorless oil. ¹H NMR (500 MHz): δ7.22-7.40 (m, 5H), 6.30 (br s, 1H), 4.37 (d, J=6.0 Hz, 2H), 3.60 (m, 2H), 2.80 (m, 1H), 2.68 (m, 2H), 2.25 (m, 2H), 1.85 (m, 2H), 1.75 (m, 1H), 1.65 (m, 2H), 1.55 (m, 2H), 1.25 (s, 6H).

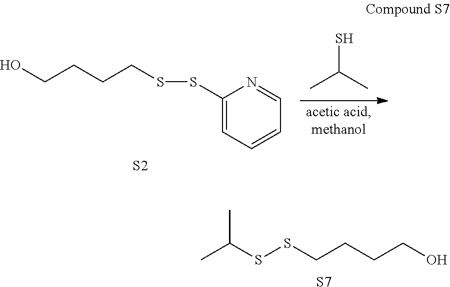

To a solution of S2 (1.0 g, 4.6 mmol) and 2-propanethiol (0.52 mL, 5.5 mmol) in 20.0 mL of methanol was added 15 μL of acetic acid. The reaction mixture was stirred for 16 hours at room temperature before being concentrated in vacuo. The crude mixture was diluted with 100 mL of ethyl acetate and washed sequentially with aqueous 1N NaOH solution (200 mL) and brine (200 mL) and then dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give 0.40 g (40%) of product S7 as colorless oil. ¹H NMR (500 MHz): δ3.63 (t, J 6.5 Hz, 2H), 2.89 (m, 1H), 2.70 (t, J 7.0 Hz, 2H), 1.80 (s, 1H), 1.75 (m, 2H), 1.65 (m, 1H), 1.27 (d, J 7.0 Hz, 6H).

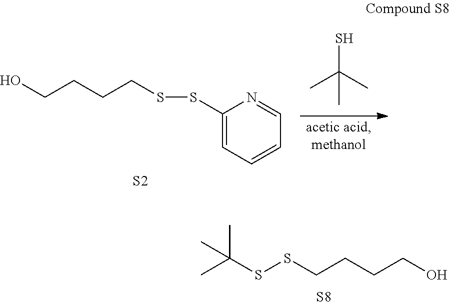

To a solution of S2 (6.0 g, 27.7 mmol) and 2-methyl-2-propanethiol (2.5 g, 27.7 mmol) in 100 mL of methanol was added 100 μL of acetic acid. The reaction mixture was stirred for 16 hours at room temperature before being concentrated in vacuo. The crude mixture was diluted with 400 mL of ethyl acetate and washed sequentially with aqueous 1N NaOH solution (200 mL) and brine (200 mL) and then dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give 3.0 g (60%) of product S8 as colorless oil. ¹H NMR (500 MHz): δ3.65 (m, 2H), 2.75 (t, J 7.5 Hz, 2H), 1.75 (m, 2H), 1.65 (m, 2H), 1.30 (s, 9H).

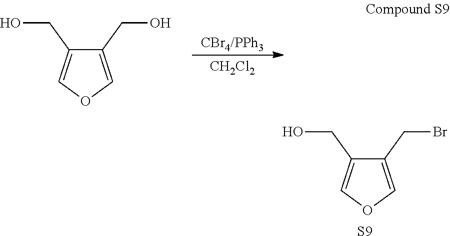 [SMILES: OCc1cocc1CO ; "CH2Cl2" ; OCc1cocc1CBr ; "S9"]

To a solution of 3,4-dishydroxymethylfuran (1.0 g, 7.8 mmol) and triphenylphosphine (2.3 g, 8.6 mmol) in 25.0 mL of dichloromethane was added carbon tetrabromide (2.85 g, 8.6 mmol). The reaction mixture was stirred for 16 hours at room temperature before being concentrated in vacuo. The crude mixture was purified by silica gel column chromatography using ethyl acetate/hexane solvent system (0-35% gradient on Combi Flash Rf Instrument) to give 1.09 g (74%) of product S9 as colorless oil which was quickly dissolved in methanol for next reaction. ¹H NMR (500 MHz): δ7.50 (s, 1H), 7.40 (s, 1H), 4.65 (s, 2H), 4.46 (s, 2H).

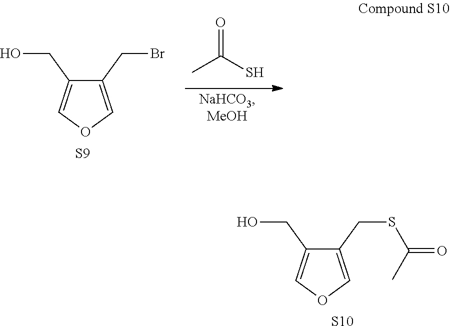

To a solution of S9 (1.09 g, 5.7 mmol) and thioacetic acid (0.52 g, 6.8 mmol) in 10.0 mL of methanol was added NaHCO₃ (0.58 g, 6.8 mmol) portion wise. The reaction mixture was stirred for 2 hours at room temperature before being neutralized to pH 7 with 1N HCl solution and the volatiles were evaporated in vacuo. The residue was diluted with 200 mL of ethyl acetate and washed sequentially with saturated NaHCO₃ solution (50 mL) and brine (50 mL) and then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 0.80 g (75%) of product S10 as colorless oil. ¹H NMR (500 MHz): δ7.37 (s, 1H), 7.35 (s, 1H), 4.53 (d, J 5.5 Hz, 2H), 4.00 (s, 2H), 2.34 (s, 3H), 1.88 (t, J 5.5 Hz, 1H).

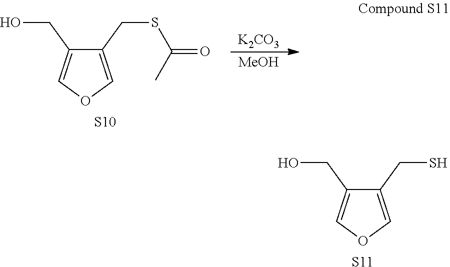

To a solution of S10 (0.60 g, 3.2 mmol) in 15.0 mL of methanol was added K₂CO₃ (0.53 g, 3.86 mmol) portion wise under Argon atmosphere. The reaction mixture was stirred for 30 minutes at room temperature before being neutralized to pH 7 with 1N HCl solution and the volatiles were evaporated in vacuo. The residue was diluted with 100 mL of ethyl acetate and washed sequentially with saturated NaHCO₃ solution (30 mL) and brine (30 mL) and then dried over anhydrous Na₂SO₄. The solvent was evaporated in vacuo and the crude mixture directly used in the next reaction.

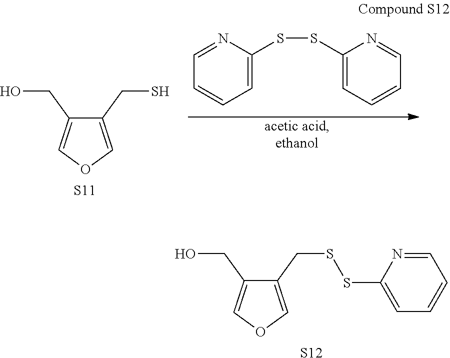

To a solution of crude S11 (0.46 g, 3.2 mmol) and dithiopyridine (0.85 g, 3.8 mmol) in 12.0 mL of ethanol was added 200 μL of acetic acid. The reaction mixture was stirred for 45 minutes at room temperature before being concentrated in vacuo. The crude mixture was purified by silica gel column chromatography using ethyl acetate/hexane solvent system (0-30% gradient on Combi Flash Rf Instrument) to give 0.40 g (50% yield) of product S12 as colorless oil. ¹H NMR (500 MHz): δ8.46 (d, J 5.0 Hz, 1H), 7.56 (m, 1H), 7.40 (d, J 8.0 Hz, 1H), 7.32 (s, 2H), 7.09 (m, 1H), 4.65 (s, 2H), 3.97 (s, 2H), 1.60 (br s, 1H).

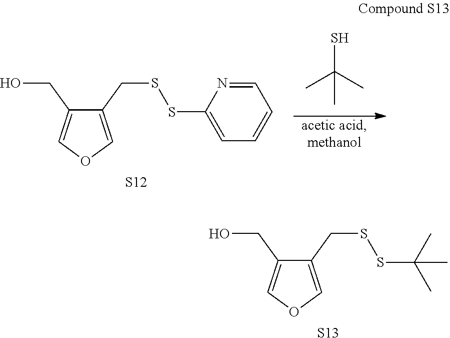

To a solution of S12 (0.39 g, 1.5 mmol) and tert-butyl mercaptan (0.21 mL, 1.8 mmol) in 20.0 mL of methanol was added 50 μL of acetic acid. The reaction mixture was stirred for 40 hours at room temperature before being concentrated in vacuo. The crude mixture was purified by silica gel column chromatography using ethyl acetate/hexane solvent system (0-50% gradient on Combi Flash Rf Instrument) to give 0.33 g (95%) of product S13 as colorless oil. ¹H NMR (500 MHz): δ7.40 (s, 1H), 7.37 (s, 1H), 4.60 (s, 2H), 3.82 (s, 2H), 1.84 (br s, 1H), 1.34 (s, 9H).

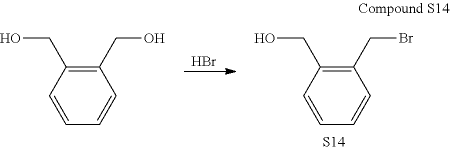

To a solution of 48% hydrobromic acid (15.0 mL) was added 1,2-benzenedimethanol (4.0 g, 29.0 mmol) and the reaction mixture was stirred for 2 hours at room temperature. 1N NaOH aqueous solution was added to the reaction mixture to neutralize the solution to pH 7. The resulting mixture was diluted with ethyl acetate (200 mL), washed sequentially by saturated NaHCO₃ solution (20 mL) and brine (20 mL), and then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 2.6 g (45%) of product S14 as white solid. ¹H NMR (500 MHz): δ7.30-7.45 (m, 4H), 4.85 (s, 2H), 4.64 (s, 2H), 1.81 (br s, 1H).

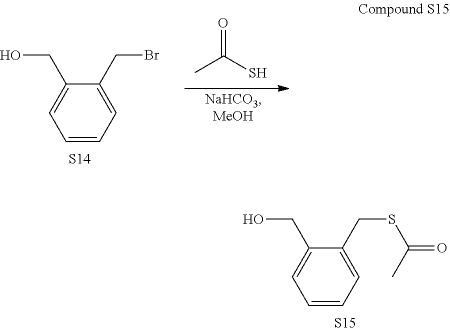

To a solution of S14 (1.0 g, 5.0 mmol) and thioacetic acid (0.46 g, 6.0 mmol) in 10.0 mL of methanol was added NaHCO₃ (0.50 g, 6.0 mmol) portion wise. The reaction mixture was stirred for 2 hours at room temperature before being neutralized to pH 7 with 1N HCl solution and the volatiles were evaporated in vacuo. The residue was diluted with 200 mL of ethyl acetate, washed sequentially by saturated NaHCO₃ solution (50 mL) and brine (50 mL), and dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 0.97 g (99%) of product S15 as colorless oil. ¹H NMR (500 MHz): δ7.40 (m, 2H), 7.25 (m, 2H), 4.73 (d, J 5.5 Hz, 2H), 4.24 (s, 2H), 2.34 (s, 3H), 2.05 (t, J 5.5 Hz, 1H).

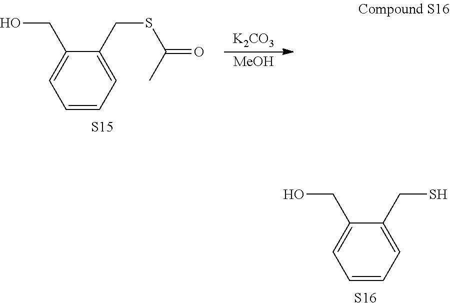

To a solution of S15 (0.75 g, 3.8 mmol) in 20.0 mL of methanol was added K₂CO₃ (0.64 g, 4.6 mmol) portion wise under argon atmosphere. The reaction mixture was stirred for 30 minutes at room temperature before being neutralized to pH 7 with 1N HCl solution, and the volatiles were evaporated in vacuo. The residue was diluted with 100 mL of ethyl acetate, washed sequentially by saturated NaHCO₃ solution (30 mL) and brine (30 mL), and dried over anhydrous Na₂SO₄. The solvent was evaporated in vacuo, and the crude product was used directly in the next reaction.

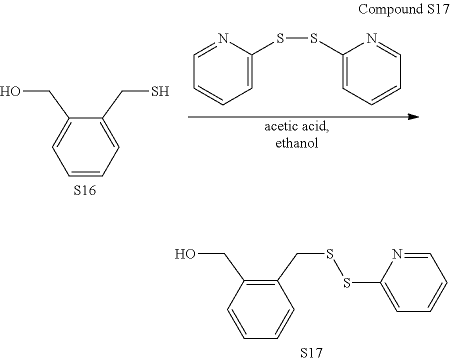

To a solution of crude S16 (0.52 g, 3.4 mmol) and dithiopyridine (0.89 mg, 4.05 mmol) in 15.0 mL of ethanol was added 0.30 mL of acetic acid. The reaction mixture was stirred for 30 minutes at room temperature before being concentrated in vacuo. The crude mixture was purified by silica gel column chromatography using ethyl acetate/hexane solvent system (0-30% gradient on Combi Flash Rf Instrument) to give 0.52 g (50%) of product S17 as colorless oil. ¹H NMR (500 MHz): δ8.42 (d, J 5.0 Hz, 1H), 7.25-7.51 (m, 7H), 4.83 (s, 2H), 4.19 (s, 2H), 3.85 (br s, 1H).

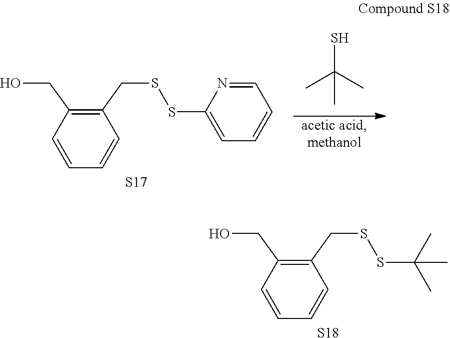

To a solution of S17 (0.42 g, 1.6 mmol) and tert-butyl mercaptan (0.21 mL, 1.9 mmol) in 20.0 mL of methanol was added 50 μL of acetic acid. The reaction mixture was stirred for 48 hours at room temperature before being concentrated in vacuo. The crude mixture was purified by silica gel column chromatography using ethyl acetate/hexane solvent system (0-50% gradient on Combi Flash Rf Instrument) to give 0.32 g (94% yield) of product S18 as colorless oil. ¹H NMR (500 MHz): δ7.40 (m, 1H), 7.26-7.30 (m, 3H), 4.80 (d, 2H, J 4.0 Hz), 4.06 (s, 2H), 1.95 (br s, 1H), 1.35 (s, 9H).

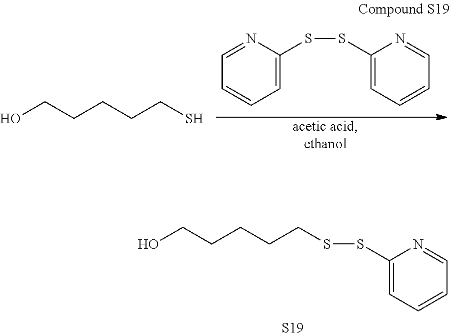

To a solution of 5-mecaptobutanol (0.85 g, 7.1 mmol) and dithiopyridine (1.87 g, 8.5 mmol) in 25.0 mL of ethanol was added 0.2 mL of acetic acid. The reaction mixture was stirred for 1 hour at room temperature before being condensed under vacuum. 50.0 mL of ethyl acetate was added to the crude product and the solution was washed sequentially by 1N NaOH aqueous solution (50 mL) and brine (30 mL) and then dried over anhydrous Na₂SO₄. The solvent was evaporated in vacuo and the crude mixture was purified by silica gel column chromatography using ethyl acetate/hexane solvent system (0-40% gradient on Combi Flash Rf Instrument) to give 1.21 g (75%) of product S19 as colorless oil. ¹H NMR (500 MHz): δ8.45 (d, J 5.0 Hz, 1H), 7.71 (d, J 8.0 Hz, 1H), 7.63 (m, 1H), 7.07 (m, 1H), 3.62 (t, J 6.5 Hz, 2H), 2.81 (t, J 7.5 Hz, 2H), 1.73 (m, 2H), 1.56 (m, 2H), 1.48 (m, 2H).

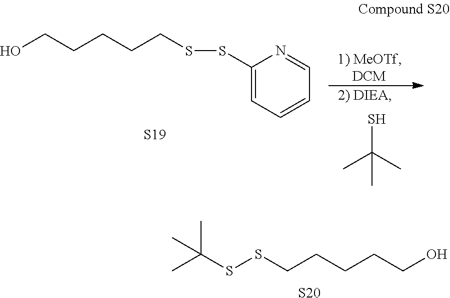

To a solution of S19 (1.2 g, 5.3 mmol) in 20.0 mL of dichloromethane was added methyl trifluoromethanesulfonate (0.87 g, 5.3 mmol). The reaction mixture was stirred for 15 minutes at room temperature followed by addition of 2-methyl-2-propanethiol (1.2 mL, 10.6 mmol) and diisopropylethalamine (DIEA) (2.7 mL, 15.9 mmol). The reaction mixture was stirred for another 1 hour before being concentrated in vacuo. The crude mixture was purified by silica gel column chromatography using ethyl acetate/hexane solvent system (0-30% gradient on Combi Flash Rf Instrument) to give 0.67 g (61%) of product S20 as colorless oil. ¹H NMR (500 MHz): δ3.65 (t, J 6.5 Hz, 2H), 2.70 (t, J 7.0 Hz, 2H), 1.67 (m, 2H), 1.57 (m, 2H), 1.45 (m, 2H), 1.32 (s, 9H).

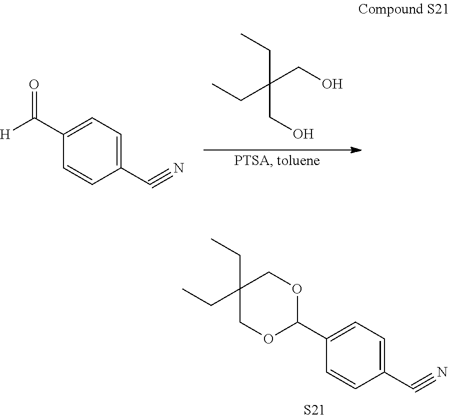

The suspension of 4-cyanobenzaldehyde (5.0 g, 38.1 mmol), 2,2-diethyl-1,3-propanediol (5.5 g, 41.9 mmol) and p-toluenesulfonic acid monohydrate (0.21 g, 1.14 mmol) in 250 mL of toluene was refluxed with Dean-Stark apparatus for 16 hours. The reaction mixture was cooled to room temperature, and the volatiles were removed under reduced pressure. The crude mixture was diluted with 300 mL of ethyl acetate, washed sequentially by saturated NaHCO₃ solution (30 mL) and brine (30 mL), and dried over anhydrous Na₂SO₄. The solvent was evaporated in vacuo, and the crude mixture was purified by silica gel column chromatography using ethyl acetate/hexane solvent system (0-20% gradient on Combi Flash Rf Instrument) to give 8.7 g (94%) of product S21 as white solid. ¹H NMR (500 MHz): δ7.66 (d, J 6.5 Hz, 2H), 7.61 (d, J 8.5 Hz, 2H), 5.4 (s, 1H), 3.97 (d, J 11.5 Hz, 2H), 3.61 (d, J 12.0 Hz, 2H), 1.79 (q, J 7.5 Hz, 2H), 1.15 (q, J 7.5 Hz, 2H), 0.89 (t, J 7.5 Hz m, 3H), 0.82 (t, J 7.5 Hz m, 3H).

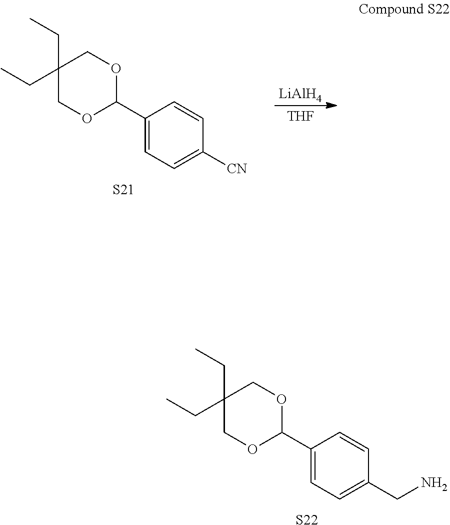 [SMILES: CCC1(CC)COC(c2ccc(C#N)cc2)OC1 ; "LiAlH4" ; "S21" ; CCC1(CC)COC(c2ccc(CN)cc2)OC1 ; "S22" ; "THF"]

The suspension of lithium aluminum hydride (0.94 g, 24.6 mmol) in THE was cooled to 0° C.° C., to which was added drop wise a solution of S21 (2.0 g, 8.2 mmol) in 25.0 mL of THE under Argon atmosphere. The reaction mixture was warmed to room temperature and further stirred for 3 hours. The suspension was cooled to 0° C.° C. by ice bath, quenched with saturated Na₂SO₄ solution and filtered through a pad of Celite®. The filtrate was concentrated under reduced pressure. The crude mixture was diluted with 100 mL of ethyl acetate, washed sequentially with saturated NaHCO₃ solution (20 mL) and brine (20 mL), and dried over anhydrous Na₂SO₄. The solvent was evaporated in vacuo to get crude intermediate S22 as colorless oil, which was used in the next reaction without further purification.

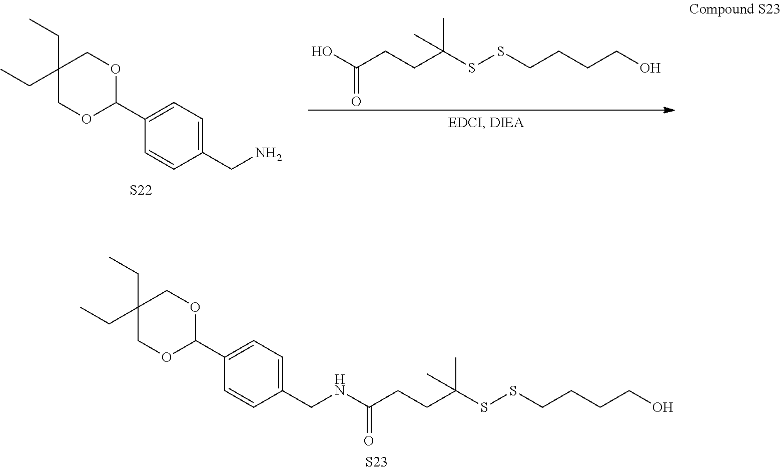 [SMILES: CCC1(CC)COC(c2ccc(CN)cc2)OC1 ; CC(C)(CCC(=O)O)SSCCCCO ; "S22" ; CCC1(CC)COC(c2ccc(CNC(=O)CCC(C)(C)SSCCCCO)cc2)OC1 ; "S23"]

To a solution of S5 (2.8 g, 11.0 mmol), EDCl (2.5 g, 13.0 mmol) and DIEA (7.6 mL, 44.0 mmol) in 25.0 mL of dichloromethane was added a solution of S22 (2.84 g, 11.0 mmol) in 10.0 mL of dichloromethane. The reaction mixture was stirred for 16 hours at room temperature before being concentrated under reduced pressure. The crude mixture was purified by silica gel column chromatography using ethyl acetate/hexane solvent system (0-40% gradient on Combi Flash Rf Instrument) to give 2.5 g (47%) of product S23 as colorless oil. ¹H NMR (500 MHz): δ7.45 (d, J 8.0 Hz, 2H), 7.26 (d, J 8.0 Hz, 2H), 5.85 (br s, 1H), 5.37 (s, 1H), 5.29 (s, 2H), 4.41 (d, J 5.5 Hz, 2H), 3.93 (d, J 11.5 Hz, 2H), 3.60 (m, 4H), 2.69 (t, J 7.5 Hz, 2H), 2.29 (m, 2H), 1.93 (m, 2H), 1.80 (q, J 7.5 Hz, 2H), 1.75 (m, 2H), 1.60 (m, 2H), 1.28 (s, 6H), 1.13 (q, J 7.5 Hz, 2H), 0.89 (t, J 7.5 Hz, 3H), 0.81 (t, J 7.5 Hz, 3H).

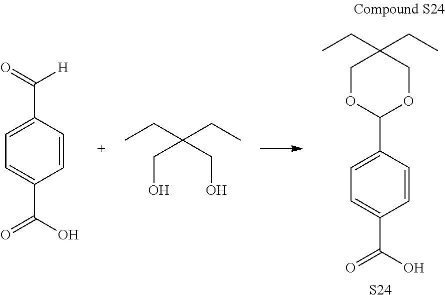

To a suspension of 4-formyl benzoic acid (15.01 g, 100 mmol) and 2,2-diethyl-1,3-propanediol (14.54 g, 110 mmol) in toluene (250 mL) was added p-toluenesulfonic acid monohydrate (0.57 g, 3.0 mmol). The mixture was refluxed overnight with a Dean-Stark apparatus. The reaction mixture was cooled to room temp to form a large amount of precipitates. The solid was filtered, heated with 100 mL of ethyl acetate and cooled to collect the precipitate, which was dried under high vacuum to give 20 g of the title compound S24. The filtrate was washed with water and brine, dried over anhydrous Na₂SO₄, and evaporated to give a white solid, which was recrystallized from ethyl acetate to give another 1.5 g of S24 (total 21.5 g, 81%). ¹H NMR (500 MHz, CDCl₃): δ8.12 (2H, d, J 8.5 Hz), 7.61 (2H, d, J 8.5 Hz), 5.45 (1H, s), 3.98 (2H, d, J 11.5 Hz), 3.62 (2H, d, J 11.5 Hz), 1.83 (2H, q, J 7.5 Hz), 1.16 (2H, q, J 7.5 Hz), 0.90 (3H, t, J 7.5 Hz), 0.83 (3H, t, J 7.5 Hz).

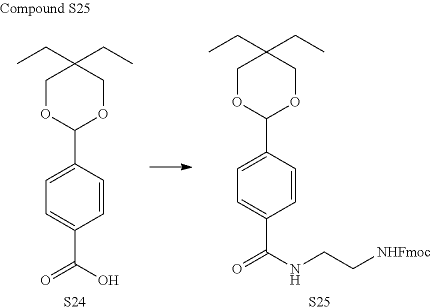

To a solution of S24 (1.32 g, 5.0 mmol) and mono-Fmoc ethylenediamine HCl salt (1.75 g, 5.5 mmol) in dimethylformamide (15.0 mL) were added HATU (2.28 g, 6.0 mmol) and N,N-diisopropylethylamine (4.35 mL, 25.0 mmol). The resulting mixture was stirred for 30 min, and the volatiles removed under high vacuum to give a brown solid. The solid was washed with ethyl acetate three times to afford 1.95 g (74%) of pure compound S25 as a white solid. ¹H NMR (500 MHz, CDCl3): δ7.78 (2H, d, J 8.0 Hz), 7.74 (2H, d, J 7.5 Hz), 7.55 (2H, d, J 7.5 Hz), 7.53 (2H, d, J 8.0 Hz), 7.37 (2H, t, J 7.5 Hz), 7.26 (2H, t, J 7.5 Hz), 7.07 (1H, br s), 5.47 (1H, br s), 5.38 (1H, s), 4.40 (2H, d, J 6.5H z), 4.16 (1H, t, J 6.5 Hz), 3.95 (2H, d, 11.5 Hz), 3.58 (2H, d, J 11.5 Hz), 3.55-3.50 (2H, m), 3.43-3.35 (2H, m), 1.81 (2H, q, J 7.5 Hz), 1.14 (2H, q, J 7.5 Hz), 0.89 (3H, t, J 7.5 Hz), 0.82 (3H, t, J 7.5 Hz)

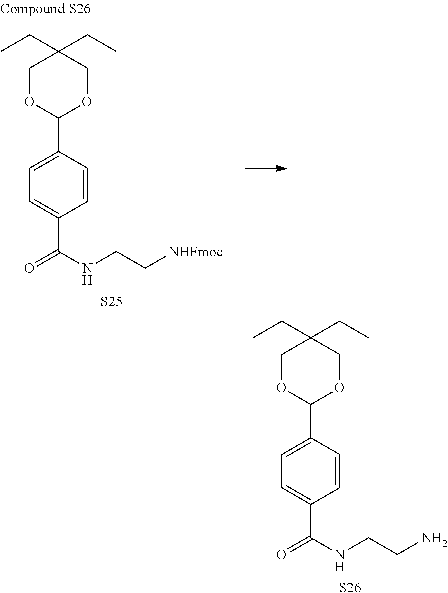

To a solution of compound S25 (1.95 g, 3.68 mmol) in dimethylformamide (15 mL) was added 3 mL of piperidine, and the mixture was stirred for 30 min. The mixture was washed with hexane (20 mL×2), and the dimethylformamide layer was evaporated under high vacuum to give crude compound S26, which was used in the next reaction without further purification.

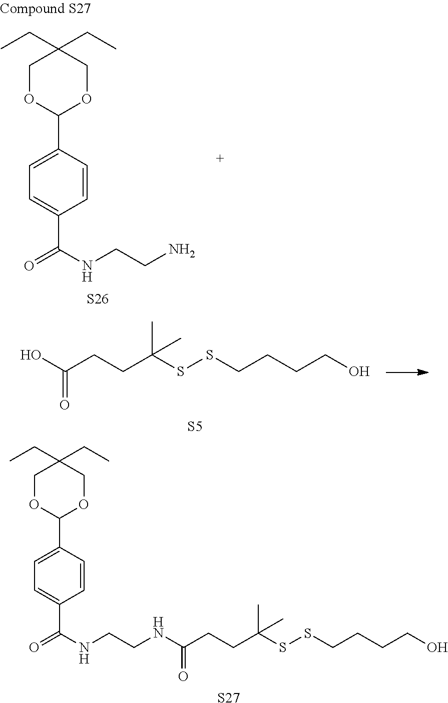

To a mixture of compound S26 and S5 (0.87 g, 3.45 mmol) in dimethylformamide (10 mL) were added HATU (1.68 g, 4.4 mmol) and N,N-diisopropylethylamine (1.2 mL, 6.9 mmol). The mixture was stirred for 1 hour, and the volatiles were removed under high vacuum to give a brown solid. The solid was washed with ethyl acetate several times and dried under high vacuum to afford 0.95 g (51%) of the title compound S27 as a white solid. ¹H NMR (500 MHz, CDCl₃): δ7.81 (2H, d, J 8.5 Hz), 7.57 (2H, d, J 8.5 Hz), 7.19 (1H, br s), 6.42 (1H, br s), 5.42 (1H, s), 3.96 (2H, d, J 11.0H z), 3.64-3.55 (6H, m), 3.53-3.47 (2H, m), 2.66 (2H, t, J 7.5 Hz), 2.31-2.26 (2H, m), 2.05 (1H, br s), 1.90-1.85 (2H, m), 1.82 (2H, q, J 7.5 Hz), 1.75-1.66 (2H, m), 1.63-1.55 (2H, m), 1.25 (6H, s), 1.15 (2H, q, J 7.5 Hz), 0.89 (3H, t, J 7.5 Hz), 0.82 (3H, t, J 7.5 Hz).

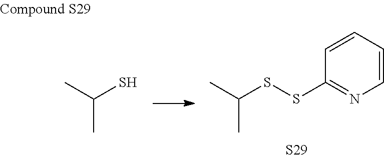

To a solution of isopropylthiol (7.6 g, 100 mmol) in ethanol (300 mL) were added dithiodipyridine (24.2 g, 110 mmol) and acetic acid (7.0 mL). The mixture was stirred overnight, and then evaporated to give a residue, which was dissolved in 200 mL of ethyl acetate. The solution was washed with 1N NaOH (50 mL×3) and brine. The organic layer was dried over anhydrous Na₂SO₄, filtered, and evaporated to give a residue, which was subjected to flash silica gel column purification on an ISCO companion (ethyl acetate/hexane=5%-20%) to give 14.4 g (77%) of the title compound S29 as a colorless oil. ¹H NMR (500 MHz, CDCl₃): δ8.44 (1H, d, J 5.0 Hz), 7.75 (1H, d, J 8.0 Hz), 7.63 (1H, td, J 8.0, 1.5 Hz), 7.06 (1H, m), 3.13 (1H, m), 1.33 (6H, d, J 7.0 Hz).

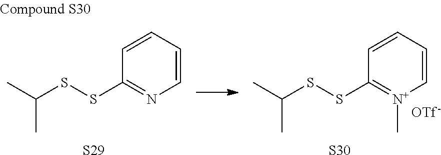

To a solution of compound S29 (1.86 g, 10.0 mmol) in dichloromethane (5.0 mL) was added MeOTf (1.64 g, 10.0 mmol). The mixture was stirred for 15 min and washed with hexane (10 mL×2). The dichloromethane layer was evaporated to give the crude salt as yellow oil (S30), which was used directly in the next reaction.

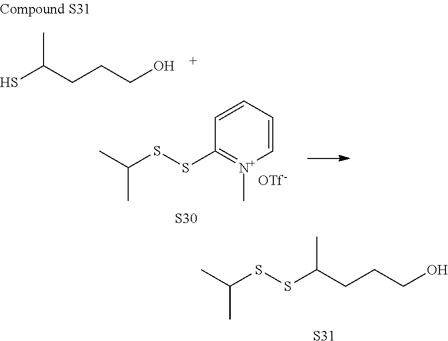

To a solution of 4-mercapto-4-methylbutan-1-ol (0.36 g, 3.0 mmol) in dichloromethane was added the crude S30 (1.26 g, 3.6 mmol) and N,N-diisopropylethylamine (1.0 mL). The mixture was stirred for 10 min, volatiles were removed under vacuum to give a residue, which was subjected to flash silica gel column purification on an ISCO companion instrument (ethyl acetate/hexane=5%-40%) to give 0.50 g (85%) of the title compound S31 as a colorless oil. ¹H NMR (500 MHz, CDCl₃): δ3.67 (2H, t, J 6.5 Hz), 2.96 (1H, J 6.5 Hz), 2.83 (1H, m), 1.77-1.67 (3H, m), 1.63-1.55 (1H, m), 1.32 (3H, d, J 6.5 Hz), 1.30 (6H, d, J 6.5 Hz).

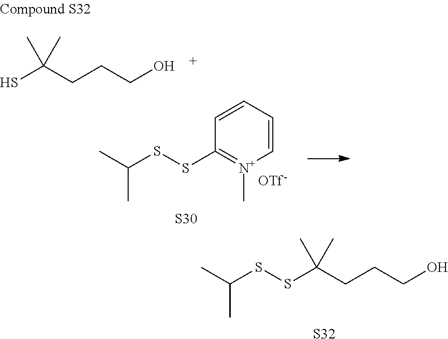

To a solution of 4-mercapto-4-methylpentan-1-ol (0.19 g, 1.39 mmol) in dichloromethane was added the crude S30 (0.58 g, 1.66 mmol) and N,N-diisopropylethylamine (1.0 mL). The mixture was stirred for 10 min, volatiles were removed under vacuum to give a residue, which was subjected to flash silica gel column purification on an ISCO companion (ethyl acetate/hexane=5%-40%) to give 0.26 g (89%) of the title compound S32 as a colorless oil. ¹H NMR (500 MHz, CDCl₃): δ3.66 (2H, t, J 5.5 Hz), 2.94 (1H, J 6.5 Hz), 1.72-1.60 (4H, m), 1.29 (6H, s), 1.29 (6H, d, J 6.5 Hz).

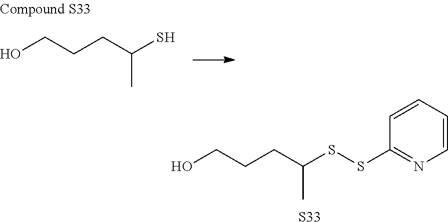

To a solution of 4-mercapto-4-methylbutan-1-ol (0.18 g, 1.5 mmol) in methanol (5.0 mL) were added dithiodipyridine (0.35 g, 1.6 mmol) and acetic acid (30 μL). The mixture was stirred for 30 min, then evaporated to give a residue, which was subjected to flash silica gel column purification on an ISCO companion (ethyl acetate/hexane=15%-70%) to give 0.27 g (78%) of the title compound S33 as a colorless oil. ¹H NMR (500 MHz, CDCl₃): δ8.84 (1H, d, J 5.0 Hz), 7.73 (1H, d, J 8.0 Hz), 7.63 (1H, td, J 8.0, 1.5 Hz), 7.07 (1H, m), 3.64 (2H, t, J 6.5 Hz), 2.99 (1H, m), 1.82-1.60 (4H, m), 1.34 (3H, d, J 7.0 Hz).

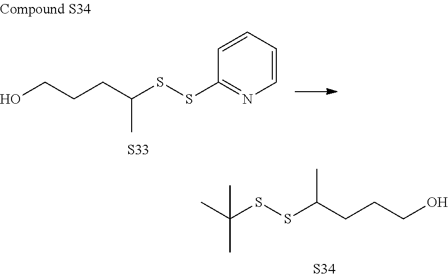

To a solution of compound S33 (0.27 g, 1.15 mmol) in dichloromethane (5.0 mL) was added MeOTf (0.19 g, 1.15 mmol). The mixture was stirred for 15 min, and then 2-methyl-2-propanethiol (0.21 g, 2.3 mmol) and N,N-diisopropylethylamine (1.0 mL) were added. The resulting mixture was stirred for another 30 min. Evaporation of the volatiles afforded a residue, which was subjected to flash silica gel column purification on an ISCO companion (ethyl acetate/hexane=5%-40%) to give 0.19 g (79%) of the title compound S34 as a colorless oil. ¹H NMR (500 MHz, CDCl₃): δ3.67 (2H, t, J 6.5 Hz), 2.84 (1H, m), 1.75-1.65 (3H, m), 1.62-1.55 (1H, m), 1.32 (9H, s), 1.31 (3H, d, J 7.0 Hz).

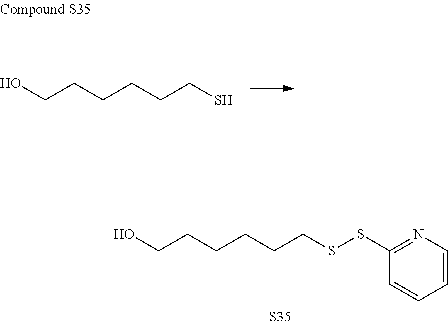 [SMILES: OCCCCCCS ; OCCCCCCSSc1ccccn1 ; "S35"]

To a solution of 6-mercapto-1-hexanol (2.68 g, 20.0 mmol) in methanol (50.0 mL) were added dithiodipyridine (6.6 g, 30.0 mmol) and acetic acid (1.0 mL). The mixture was stirred for 30 min and then evaporated to give a residue, which was subjected to flash silica gel column purification on an ISCO companion (ethyl acetate/hexane=15%-70%) to give 4.37 g (90%) of the title compound S35 as a colorless oil. ¹H NMR (500 MHz, CDCl₃): δ8.46 (1H, d, J 4.5 Hz), 7.72 (1H, d, J 8.0 Hz), 7.64 (1H, td, J 8.0, 1.5 Hz), 7.07 (1H, m), 3.63 (2H, t, J 6.5 Hz), 2.80 (2H, t, J 7.0 Hz), 1.72 (2H, p, J 7.5 Hz), 1.60-1.53 (2H, m), 1.47-1.40 (2H, m), 1.39-1.34 (2H, m).

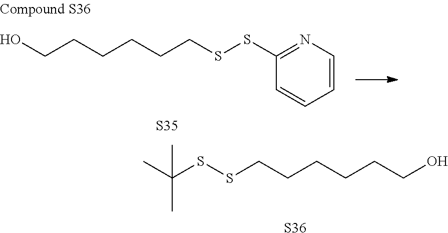

To a solution of compound S35 (1.0 g, 4.1 mmol) in dichloromethane (15.0 mL) was added MeOTf (0.67 g, 4.1 mmol). The mixture was stirred for 15 min, and then 2-methyl-2-propanethiol (0.9 mL, 8.2 mmol) and N,N-diisopropylethylamine (2.0 mL) were added. The resulting mixture was stirred for another 30 min. Evaporation of the volatiles afforded a residue, which was subjected to flash silica gel column purification on an ISCO companion (ethyl acetate/hexane=5%-60%) to give 0.61 g (67%) of the title compound S36 as a colorless oil. ¹H NMR (500 MHz, CDCl₃): δ3.65 (2H, t, J 6.5 Hz), 2.70 (2H, t, J 7.0 Hz), 1.70-1.64 (2H, m), 1.62-1.55 (2H, m), 1.45-1.35 (4H, m), 1.33 (9H, s).

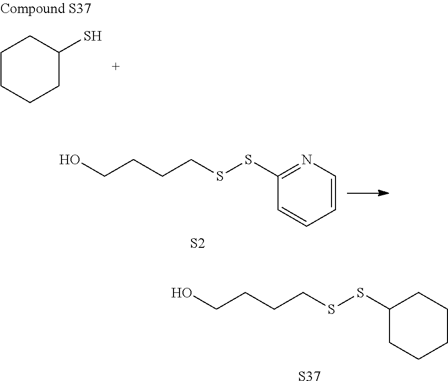 [SMILES: SC1CCCCC1 ; OCCCCSSc1ccccn1 ; "S2" ; OCCCCSSC1CCCCC1 ; "S37"]

To a solution of compound S2 (0.43 g, 2.0 mmol) in dichloromethane (10.0 mL) was added MeOTf (0.33 g, 2.0 mmol). The mixture was stirred for 15 min, and then cyclohexanethiol (0.23 g, 2.0 mmol) and N,N-diisopropylethylamine (1.0 mL) were added. The resulting mixture was stirred for another 30 min.

Evaporation of the volatiles afforded a residue, which was subjected to flash silica gel column purification on an ISCO companion (ethyl acetate/hexane=5%-60%) to give 0.36 g (81%) of the title compound S37 as a colorless oil. ¹H NMR (500 MHz, CDCl₃): δ3.67 (2H, t, J 6.5 Hz), 2.74-2.68 (1H, m), 2.71 (1H, t, J 7.0 Hz), 2.05-2.00 (2H, m), 1.81-1.74 (4H, m), 1.71-1.65 (2H, m), 1.65-1.58 (1H, m), 1.40-1.20 (6H, m).

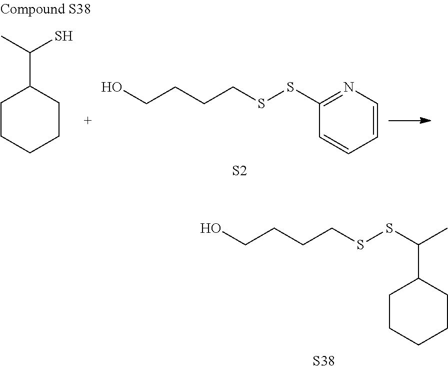

To a solution of compound S2 (0.65 g, 3.0 mmol) in dichloromethane (12.0 mL) was added MeOTf (0.49 g, 3.0 mmol). The mixture was stirred for 15 min, and then 1-cyclohexylethane-1-thiol (0.42 g, 3.6 mmol) and N,N-diisopropylethylamine (1.0 mL) were added. The resulting mixture was stirred for another 30 min. Evaporation of the volatiles afforded a residue, which was subjected to flash silica gel column purification on an ISCO companion (ethyl acetate/hexane=5%-60%) to give 0.58 g (78%) of the title compound S38 as a colorless oil. ¹H NMR (500 MHz, CDCl₃): δ3.68 (2H, t, J 6.5 Hz), 2.75-2.65 (1H, m), 2.70 (2H, t, J 7.0 Hz), 1.82-1.72 (6H, m), 1.70-1.63 (3H, m), 1.58-1.52 (1H, m), 1.28 (3H, d, J 7.0 Hz), 1.30-1.05 (5H, m).

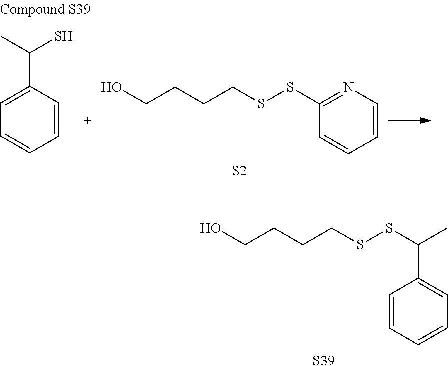

To a solution of compound S2 (0.43 g, 2.0 mmol) in methanol (5.0 mL) were added benzylethane-1-thiol (0.28 g, 2.0 mmol) and acetic acid (30 μL). The resulting mixture was stirred overnight. Evaporation of the volatiles afforded a residue, which was subjected to flash silica gel column purification on an ISCO companion (ethyl acetate/hexane=5%-60%) to give 0.24 g (50%) of the title compound S39 as a colorless oil. ¹H NMR (500 MHz, CDCl₃): δ7.38-7.30 (4H, m), 7.27-7.23 (1H, m), 3.59 (2H, t, J 6.5 Hz), 2.30 (2H, t, J 7.0 Hz), 1.67 (3H, d, J 7.0 Hz), 1.62-1.51 (4H, m).

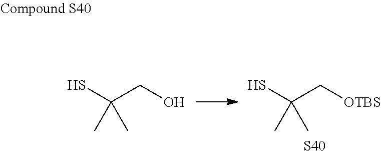

To a solution of 2-mercapto-2-methylpropan-1-ol (0.50 g, 4.7 mmol) in dichloromethane (15.0 mL) were added TBDMSCl (0.75 g, 4.9 mmol) and imidazole (0.48 g, 7.1 mmol) at 0° C. and stirred for 30 min forming large amount of white precipitates. The white solid was filtered off and washed with 10 mL of dichloromethane. The filtrate was evaporated to give a residue, which was subjected to flash silica gel column purification on an ISCO companion (ethyl acetate/hexane=0%-30%) to give 0.66 g (64%) of the title compound S40 as a colorless oil. ¹H NMR (500 MHz, CDCl₃): δ3.47 (2H, s), 1.32 (6H, s), 0.92 (9H, s), 0.07 (6H, s).

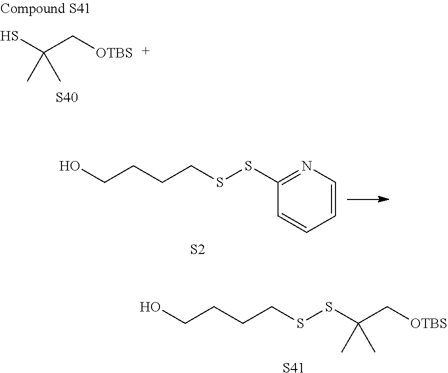 [SMILES: CC(C)(S)CO[Si](C)(C)C(C)(C)C ; "S40" ; OCCCCSSc1ccccn1 ; "S2" ; CC(C)(CO[Si](C)(C)C(C)(C)C)SSCCCCO ; "S41"]

To a solution of compound S2 (0.78 g, 3.6 mmol) in dichloromethane (12.0 mL) was added MeOTf (0.59 g, 3.6 mmol). The mixture was stirred for 15 min, and then S40 (0.66 g, 3.0 mmol) and N,N-diisopropylethylamine (1.0 mL) were added. The resulting mixture was stirred for another 30 min. Evaporation of the volatiles afforded a residue, which was subjected to flash silica gel column purification on an ISCO companion (ethyl acetate/hexane=5%-60%) to give 0.80 g (82%) of the title compound S41 as a colorless oil. ¹H NMR (500 MHz, CDCl₃): δ3.58 (2H, t, J 6.5 Hz), 3.41 (2H, s), 2.62 (2H, t, J 7.0 Hz), 1.70-1.63 (2H, m), 1.62-1.55 (2H, m), 1.17 (6H, s), 0.81 (9H, s), 0.03 (6H, s).

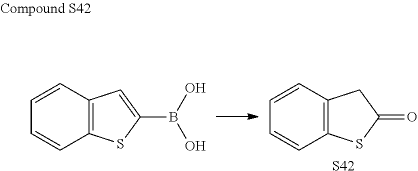 [SMILES: OB(O)c1cc2ccccc2s1 ; O=C1Cc2ccccc2S1 ; "S42"]

To a solution of thianaphthene-2-boronic acid (3.09 g, 17.0 mmol) in EtOH (30.0 mL) was added hydrogen peroxide (30%, 5.6 mL) dropwise. After stirring overnight, the reaction mixture was carefully concentrated under reduced pressure, diluted with water (100 mL), and extracted with ethyl acetate (70 mL×3). The combined organic layer was dried over anhydrous sodium sulfate and concentrated in vacuo to afford a residue, which was subjected to flash silica gel column purification on a ISCO companion (ethyl acetate/hexane=0%-20%) to give 2.17 g (85%) of the title compound S42 as a colorless oil. ¹H NMR (500 MHz, CDCl₃): δ7.34 (1H, dd, J 8.5 Hz), 7.31-7.28 (2H, m), 7.22 (1H, td, J 8.0, 1.0 Hz), 3.98 (2H, s).

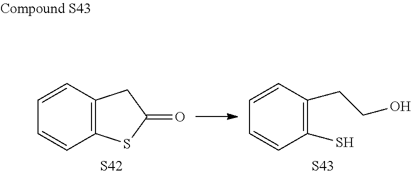 [SMILES: O=C1Cc2ccccc2S1 ; OCCc1ccccc1S ; "S42" ; "S43"]

To a solution of LiAlH₄ (1.1 g, 28.8 mmol) in THE (40.0 mL) was added a solution of compound S42 (2.16 g, 14.4 mmol) in THF. The mixture was stirred overnight and the reaction mixture was quenched with water (20 mL) carefully while cooling to 0° C., followed by addition of 50 mL of 1N HCl. The phases were separated, and the aqueous layer was extracted with ethyl acetate (2×50 mL). The combined organic layers were washed with brine, dried over anhydrous sodium sulfate, and concentrated in vacuo to afford a residue, which was subjected to flash silica gel column purification on a ISCO companion (ethyl acetate/hexane=10%-50%) to give 0.69 g (31%) of the title compound S43 as a colorless oil. ¹H NMR (500 MHz, CDCl₃): δ7.31 (1H, dd, J 7.5, 1.5 Hz), 7.20 (1H, dd, J 7.5, 1.5 Hz), 7.16-7.08 (2H, m), 3.91 (2H, t, J 6.5 Hz), 3.41 (1H, s), 2.98 (1H, J 6.5 Hz).

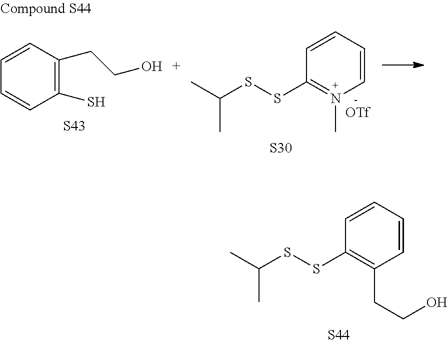

To a solution of compound S43 (0.23 g, 1.5 mmol) in dichloromethane (5.0 mL) were added the disulfide pyridinium salt S30 (0.70 g, 2.0 mmol) and N,N-diisopropylethylamine (1.0 mL). The mixture was stirred for 10 min, and the volatiles were removed under vacuum to give a residue, which was subjected to flash silica gel column purification on a ISCO companion (ethyl acetate/hexane=5%-50%) to give 0.29 g 30 (85%) of the title compound S44 as a colorless oil. ¹H NMR (500 MHz, CDCl₃): δ7.79 (1H, d, J 8.0 Hz), 7.27-7.23 (1H, m), 7.21-7.18 (2H, m), 3.91 (2H, t, J 6.5 Hz), 3.10 (2H, t, J 6.5 Hz), 3.07-3.03 (1H, m), 1.30 (6H, d, J 7.0 Hz).

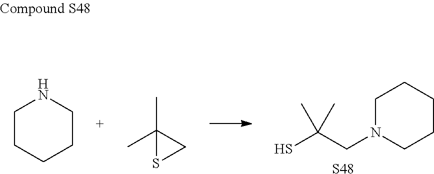

The mixture of isobutylene sulfide (0.88 g, 10.0 mmol) and piperidine (0.84 mL, 8.5 mmol) was heated to 80° C. and stirred for 4 hours. Evaporation of the volatiles afforded the crude product S48, which was used directly in the next step without purification.

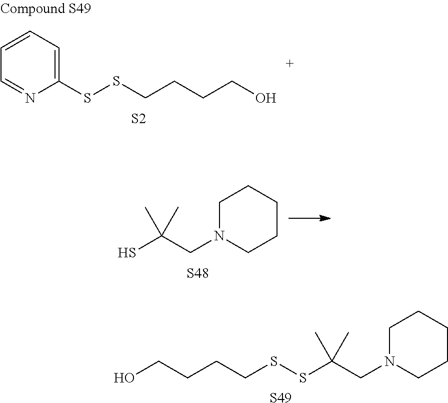

To a solution of compound S2 (0.65 g, 3.0 mmol) in dichloromethane (12.0 mL) was added MeOTf (0.49 g, 3.0 mmol). The mixture was stirred for 15 min, and then the crude S48 (0.49 g, 3.0 mmol) and diisopropylethylamine (1.0 mL) were added. The resulting mixture was stirred for another 30 min. Evaporation of the volatiles afforded a residue, which was subjected to flash silica gel column purification on an ISCO companion (ethyl acetate/hexane=5%-60%) to give 0.50 g (52% for two steps) of the title compound S49 as a colorless oil. ¹H NMR (500 MHz, CDCl₃): δ3.69 (2H, m), 2.72 (2H, t, J 7.0 Hz), 2.49 (4H, m), 2.37 (2H, s), 1.80-1.70 (2H, m), 1.70-1.62 (2H, m), 1.55-1.47 (4H, m), 1.40-1.34 (2H, m), 1.27 (6H, s).

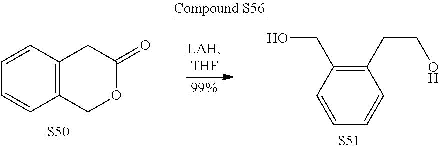 [SMILES: O=C1Cc2ccccc2CO1 ; OCCc1ccccc1CO ; "S50" ; "S51"]

The suspension of lithium aluminum hydride (1.03 g, 27.0 mmol) in THE was cooled to 0° C., to which was added drop wise a solution of 3-isochromanone S50 (2.0 g, 13.5 mmol) in 25 mL of THE under argon atmosphere. The reaction mixture was warmed up to room temperature and further stirred for 3 hours. The suspension was cooled to 0° C. again by ice bath, quenched with saturated Na₂SO₄ solution and filtered through a pad of Celite®. The filtrate was concentrated under reduced pressure. The crude mixture was diluted with 100 mL of ethyl acetate, washed sequentially with saturated NaHCO₃ solution (20.0 mL) and brine (20.0 mL), and dried over anhydrous Na₂SO₄. The solvent was evaporated in vacuo to get intermediate S51 as a colorless oil (2.01 g, 99% yield), which was used directly in the next step without further purification. ¹H NMR (500 MHz): δ7.34-7.22 (m, 4H), 4.65 (s, 2H), 3.89 (t, J 6.0 Hz, 2H), 2.96 (t, J 6.0 Hz, 2H)

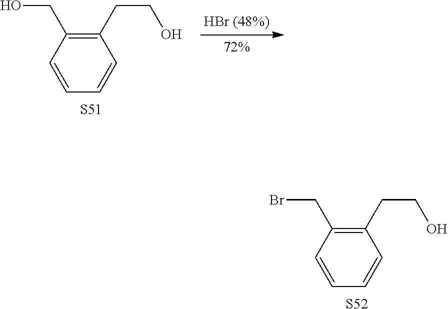 [SMILES: OCCc1ccccc1CO ; "S51" ; OCCc1ccccc1CBr ; "S52"]

To intermediate S51 (4.0 g, 26.5 mmol) was added a solution of 48% hydrobromic acid (20.0 mL) drop wise. The reaction mixture was stirred for 3 hours at room temperature before being poured into ice water. The resulting mixture was extracted with ethyl ether (200 mL), washed sequentially with saturated NaHCO₃ solution (20.0 mL) and brine (20.0 mL), and dried over anhydrous Na₂SO₄. The solvent was evaporated in vacuo to give intermediate S52 as a light yellow oil (4.2 g, 72% yield), which was used directly in the next step without further purification. ¹H NMR (500 MHz): δ7.37-7.15 (m, 4H), 4.59 (s, 2H), 3.94 (t, J 6.5 Hz, 2H), 3.03 (t, J 6.5 Hz, 2H)

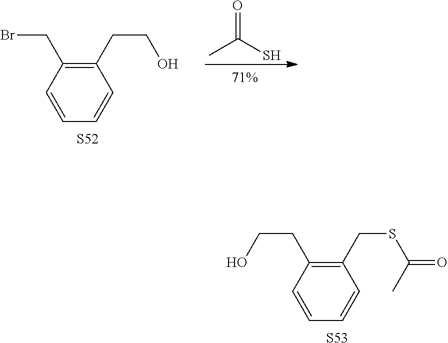

To a solution of S52 (5.5 g, 25.6 mmol) and thioacetic acid (2.24 g, 30.7 mmol) in 50.0 mL of methanol was added NaHCO₃ (2.58 g, 30.7 mmol) portionwise. The reaction mixture was stirred for 2 hours at room temperature before neutralized to pH 7 with 1 N HCl solution, and the volatiles evaporated in vacuo. The residue was diluted with 300 mL of ethyl acetate, washed with brine (50.0 mL), and dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give product S53 as a light yellow oil (3.8 g, 71% yield). ¹H NMR (500 MHz): δ7.30-7.18 (m, 4H), 4.20 (s, 2H), 3.87 (t, J 7.0 Hz, 2H), 2.92 (t, J 7.0 Hz, 2H), 2.34 (s, 3H)

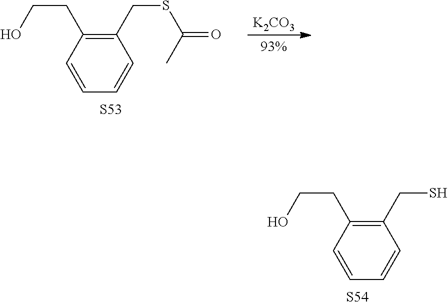

To a solution of S53 (3.8 g, 18.1 mmol) in 50 mL of methanol was added K₂CO₃ (3.0 g, 21.7 mmol) portion wise under argon atmosphere. The reaction mixture was stirred for 30 minutes at room temperature before being neutralized to pH 7 with 1 N HCl solution, and the volatiles were evaporated in vacuo. The residue was diluted with 200 mL of ethyl acetate, washed with brine (50.0 mL), and dried over anhydrous Na₂SO₄. The solvent was evaporated in vacuo to give crude product S54 as light yellow oil (2.8 g, 93% yield), which was used directly in the next step reaction without further purification.

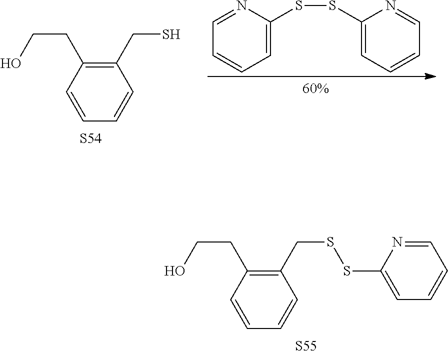 [SMILES: OCCc1ccccc1CS ; c1ccc(SSc2ccccn2)nc1 ; "S54" ; OCCc1ccccc1CSSc1ccccn1 ; "S55"]

To a solution of crude S54 (2.8 g, 16.7 mmol) and dithiopyridine (4.4 g, 20.0 mmol) in 50.0 mL of ethanol was added 1.0 mL of acetic acid. The reaction mixture was stirred for 3 hours at room temperature before being concentrated in vacuo. The crude mixture was purified by silica gel column chromatography using ethyl acetate/hexane solvent system (0-50% gradient on Combi Flash Rf Instrument) to give product S55 as colorless oil (2.5 g, 60% yield). ¹H NMR (500 MHz): δ8.43 (d, J 4.5 Hz, 1H), 7.58-7.55 (m, 2H), 7.26-7.07 (m, 5H), 4.14 (s, 2H), 3.96 (t, J 6.5 Hz, 2H), 3.04 (t, J 6.5 Hz, 2H)

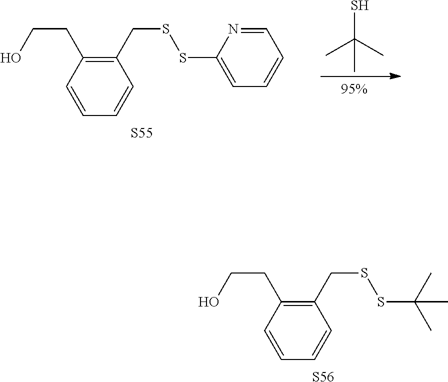

To a solution of S55 (1.14 g, 4.1 mmol) and tert-butyl mercaptan (560 μL, 4.9 mmol) in 25 mL of methanol was added 100 μL of acetic acid. The reaction mixture was stirred for 48 hours at room temperature before being concentrated in vacuo. The crude mixture was purified by silica gel column chromatography using ethyl acetate/hexane solvent system (0-50% gradient on Combi Flash Rf Instrument) to give product S56 as colorless oil (0.90 g, 97% yield, 0.14 g of S55 was recovered). ¹H NMR (500 MHz): δ7.29-7.20 (m, 4H), 4.03 (s, 2H), 3.92 (t, J 6.5 Hz, 2H), 3.01 (t, J 6.5 Hz, 2H), 1.36 (s, 9H)

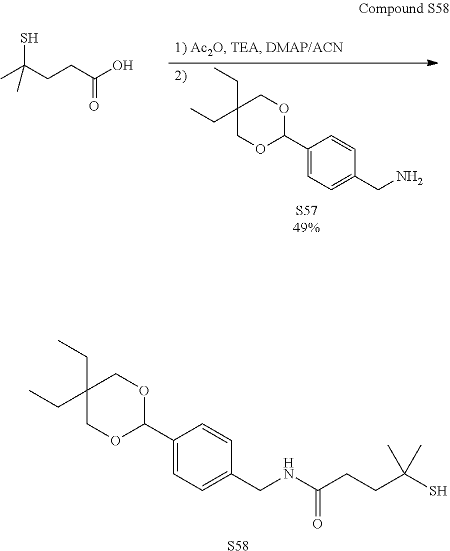

To a solution of 4-sulfanyl-4-methylpentanoic acid (5.0 g, 33.7 mmol) and acetic anhydride (3.5 mL, 37.1 mmol) in 30.0 mL of acetonitrile under argon atmosphere was added triethylamine (9.4 mL, 67.4 mmol) and a catalytic amount of DMAP. The reaction mixture was stirred at room temperature for 30 min, at which time intermediate S57 (12.6 g, 50.55 mmol) in 15.0 mL of acetonitrile was added. The reaction mixture was stirred at room temperature overnight before being concentrated in vacuo. The crude mixture was purified by silica gel column chromatography using ethyl acetate/hexane solvent system (0-50% gradient on Combi Flash Rf Instrument) to give product S58 as light yellow oil (6.2 g, 49% yield). ¹H NMR (500 MHz): δ7.32 (d, J 8.5 Hz, 2H), 7.26 (d, J 8.5 Hz, 2H), 5.7 (brs, 1H), 5.37 (s, 1H), 4.41 (d, J 5.5 Hz, 2H), 3.94 (d, J 11.5 Hz, 2H), 3.58 (d, J 11.5 Hz, 2H), 2.37 (m, 2H), 1.93 (m, 2H), 1.81 (q, J 7.5 Hz, 2H), 1.38 (s, 6H), 1.13 (q, J 8.0 Hz, 2H), 0.89 (t, J 7.5 Hz, 3H), 0.81 (t, J 8.0 Hz, 3H), 1.83 (m, 2H), 1.70 (m, 2H), 1.62 (m, 2H), 1.25 (s, 6H)

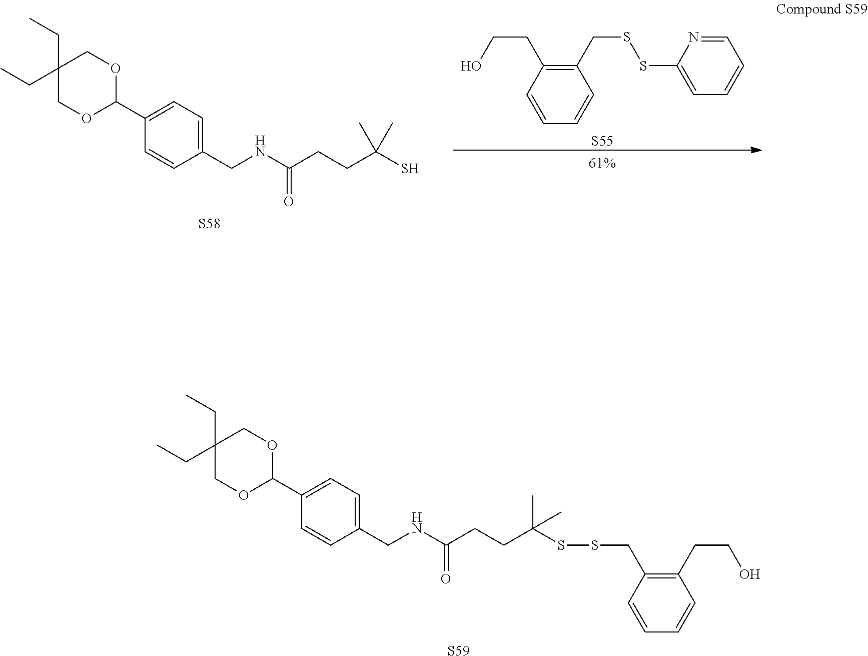 [SMILES: "S55" ; CCC1(CC)COC(c2ccc(CNC(=O)CCC(C)(C)S)cc2)OC1 ; OCCc1ccccc1CSSc1ccccn1 ; "S58" ; CCC1(CC)COC(c2ccc(CNC(=O)CCC(C)(C)SSCc3ccccc3CCO)cc2)OC1 ; "S59"]

To a solution of S55 (0.50 g, 1.8 mmol) and S58 (0.68 g, 1.8 mmol) in 10.0 mL of methanol was added 100 μL of acetic acid. The reaction mixture was stirred for 16 hours at room temperature before being concentrated in vacuo. The crude mixture was purified by silica gel column chromatography using ethyl acetate/hexane solvent system (0-50% gradient on Combi Flash Rf Instrument) to give product S59 as light yellow oil (0.60 g, 61% yield). ESI MS for C₃₀H₄₃NO₄S₂ calculated 545, observed [M+H]⁺ 546. ¹H NMR (500 MHz): δ7.44 (d, J 8.0 Hz, 2H), 7.30-7.18 (m, 6H), 5.78 (brs, 1H), 5.36 (s, 1H), 4.41 (d, J 5.5 Hz, 2H), 4.07 (s, 2H), 3.93 (d, J 11.5 Hz, 2H), 3.81 (brs, 2H), 3.58 (d, J 11.5 Hz, 2H), 3.02 (t, J 7.5 Hz, 2H), 2.86 (brs, 1H), 2.34 (m, 2H), 2.05 (m, 2H), 1.81 (q, J 7.5 Hz, 2H), 1.30 (s, 6H), 1.13 (q, J 8.0 Hz, 2H), 0.89 (t, J 8.0 Hz, 3H), 0.81 (t, J 7.5 Hz, 3H)

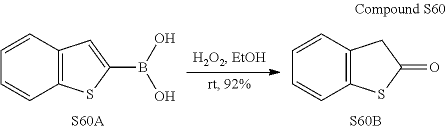 [SMILES: OB(O)c1cc2ccccc2s1 ; O=C1Cc2ccccc2S1 ; "S60B"]

To a solution of compound S60A (30.0 g, 168.5 mmol) in EtOH (120 mL) was added 30% hydrogen peroxide (50 mL) dropwise over 45 min (caution: exothermic). Reaction mixture became turbid with white precipitate. TLC showed completion of the reaction at 3 h, at which time the reaction mixture was diluted with water (300 mL), and carefully extracted with dichloromethane (200 mL×3). The combined organic layer was dried over anhydrous sodium sulfate and concentrated in vacuo to afford crude product. This was purified by flash silica gel column (220 g) using ISCO companion (ethyl acetate/hexane, 0-20% over 15 column volumes) to give 23.5 g (92%) of compound S60B as a light yellow oil which became solid on standing at room temperature. ¹H NMR (500 MHz, CDCl₃): δ7.34 (1H, dd, J 8.5 Hz), 7.31-7.28 (2H, m), 7.22 (1H, td, J 8.0, 1.0 Hz), 3.98 (2H, s)

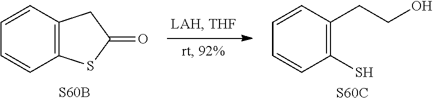 [SMILES: "S60B" ; "S60C" ; O=C1Cc2ccccc2S1 ; OCCc1ccccc1S]

To an ice cold solution of LiAlH₄ (7.4 g, 200.0 mmol) in diethyl ether (200 mL) was added dropwise a solution of compound S60B (15.0 g, 100.0 mmol) in diethyl ether over 1 hr (caution: gas evolution and exothermic). The reaction mixture was allowed to reach room temperature and stirring was continued overnight. TLC showed completion of reaction, at which time the reaction mixture was carefully quenched by addition of aq. sodium sulfate until gas evolution stopped and the formation of a white precipitate ceased. To this mixture, was carefully added 100 mL of 10% H₂SO₄ and the layers were separated. The aqueous layer was extracted with 3×75 mL ether, and the combined organic layers were washed with water, brine, dried over sodium sulfate, and concentrated to give compound S60C (14.6 g, 95%) as colorless oil, which was used in the next reaction without further purification. ¹H NMR (500 MHz, CDCl₃): δ7.31 (1H, dd, J 7.5, 1.5 Hz), 7.20 (1H, dd, J 7.5, 1.5 Hz), 7.16-7.08 (2H, m), 3.91 (2H, t, J 6.5 Hz), 3.41 (1H, s), 2.98 (1H, J 6.5 Hz)

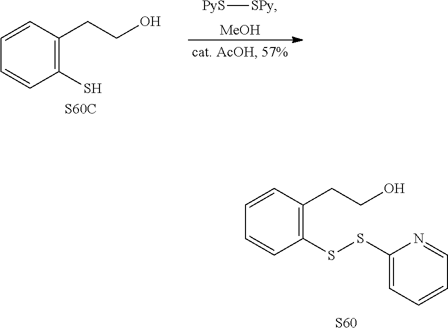

To a solution of dithiodipyridine (52.0 g, 236.3 mmol) and acetic acid (3.0 mL) in methanol (200 mL) at room temperature was added a solution of compound S60C (14.6 g, 94.5 mmol) in methanol (50 mL) and stirred overnight. Volatiles were removed, and to the residue were added 100 mL of diethyl ether. The separated solids were filtered and washed with diethyl ether (3×50 mL). The combined ether washings were concentrated to give crude product, which, on flash silica gel column purification using ISCO companion (ethyl acetate/hexane, 0-50%), gave 14.1 g (57%) of compound S60. ¹H NMR (500 MHz, CDCl₃): δ8.48 (1H, d, J 5.0 Hz), 7.65-7.60 (3H, m), 7.25-7.18 (3H, m), 7.13-7.10 (1H, m), 3.96 (2H, t, J 6.5 Hz), 3.17 (1H, t, J 6.5 Hz)

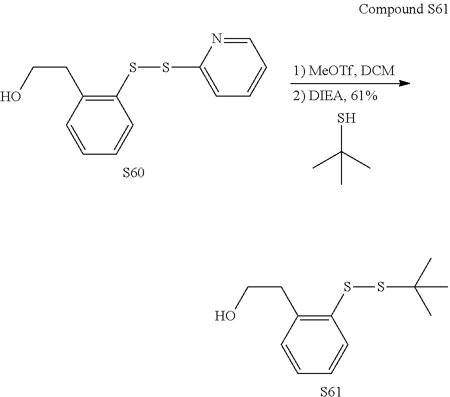

To a solution of compound S60 (4.5 g, 17.0 mmol) in 30.0 mL of dichloromethane was added MeOTf drop wise at room temperature. The reaction mixture was stirred for 10 minutes before tert-butyl mercaptan (1.9 mL, 17.0 mmol) and DIEA (6.0 mL, 34.0 mmol) were added. The reaction mixture was stirred for another 30 min at room temperature before being concentrated in vacuo. The crude mixture was purified by silica gel column chromatography using ethyl acetate/hexane solvent system (0-30% gradient on Combi Flash Rf Instrument) to give product S61 as colorless oil (2.5 g, 61% yield). ¹H NMR (500 MHz): δ7.84 (d, J 5.0 Hz, 1H), 7.25-7.13 (m, 3H), 3.92 (t, J 7.0 Hz, 2H), 3.12 (t, J 7.0 Hz, 2H), 1.30 (s, 9H)

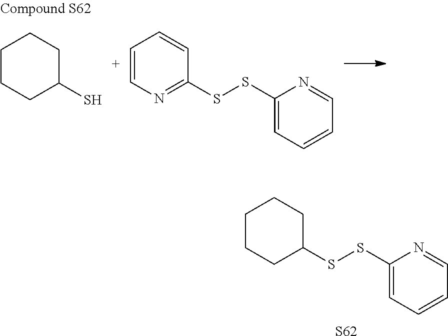

Compound S62 was prepared according to the procedure described for compound S55 using AcOH activator as reported above. ¹H NMR (500 MHz, CDCl₃): δ8.45 (1H, s), 7.78 (1H, d, J 8.0 Hz), 7.64 (1H, t, J 8.0 Hz), 7.09-7.04 (1H, m), 2.90-2.80 (1H, m), 2.06-1.98 (2H, m), 1.80-1.73 (2H, m), 1.63-1.56 (1H, m), 1.45-1.35 (2H, m), 1.33-1.18 (3H, m)

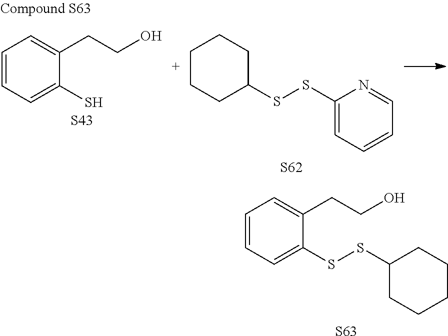 [SMILES: "S43" ; OCCc1ccccc1S ; c1ccc(SSC2CCCCC2)nc1 ; "S62" ; OCCc1ccccc1SSC1CCCCC1 ; "S63"]

Compound S63 was prepared according to the procedure described for compound S41 using MeOTf activator as reported above. ¹H NMR (500 MHz, CDCl₃): δ7.80 (1H, d, J=8.0 Hz), 7.30-7.23 (1H, m), 7.21-7.17 (2H, m), 3.90 (2H, t, J 6.5 Hz), 3.09 (2H, t, J 6.5 Hz), 2.82-2.70 (1H, m), 2.06-1.98 (2H, m), 1.80-1.72 (2H, m), 1.63-1.55 (1H, m), 1.41-1.18 (5H, m)

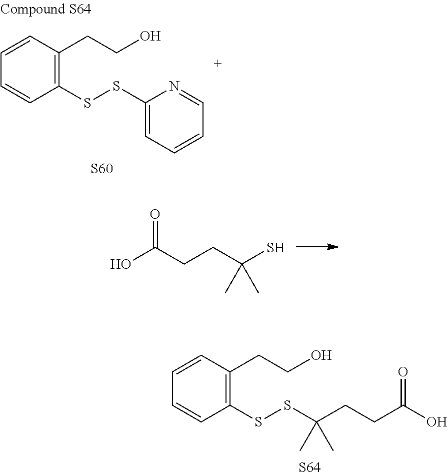

Compound S64 was prepared according to the procedure described for compound S41 using MeOTf activator as reported above. ¹H NMR (500 MHz, CDCl₃): δ7.81 (1H, d, J 8.0 Hz), 7.26-7.21 (1H, m), 7.19-7.13 (2H, m), 3.93 (2H, t, J 6.5 Hz), 3.13 (2H, t, J 6.5 Hz), 2.38-2.34 (2H, m), 1.90-1.86 (2H, m), 1.27 (1H, s)

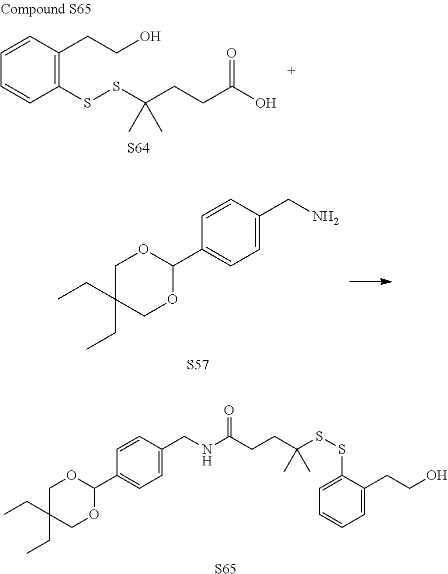 [SMILES: CC(C)(CCC(=O)O)SSc1ccccc1CCO ; "S64" ; CCC1(CC)COC(c2ccc(CN)cc2)OC1 ; "S57" ; CCC1(CC)COC(c2ccc(CNC(=O)CCC(C)(C)SSc3ccccc3CCO)cc2)OC1 ; "S65"]

To a mixture of compound S57 (1.13 g, 4.54 mmol) and S64 (1.24 g, 4.13 mmol) in DMF (12 mL) were added HCTU (2.56 g, 6.20 mmol) and N,N-diisopropylethylamine (1.76 mL, 10.3 mmol). The mixture was stirred for 1 hour and the volatiles were removed under high vacuum to give a residue, which was subjected to flash silica gel column purification on a ISCO companion (ethyl acetate/hexane, 10-70%) to give 1.28 g (58%) of the title compound S65 as a colorless oil. ¹H NMR (500 MHz, CDCl₃): δ7.81 (1H, d, J 8.0 Hz), 7.47 (2H, d, J 8.0 Hz), 7.21-7.10 (3H, m), 7.07 (1H, t, J 7.5 Hz), 7.01 (1H, d, J 7.5 Hz), 5.40 (1H, s), 4.92 (1H, s, br), 4.24 (2H, d, J 5.5 Hz), 3.96 (2H, d, J 11.5 Hz), 3.73 (2H, t, J 6.5 Hz), 3.61 (2H, d, J 11.5 Hz), 2.97 (2H, t, J 6.5 Hz), 2.10-2.02 (2H, m), 1.84 (2H, q, J 7.5 Hz), 1.81-1.76 (2H, m), 1.29 (6H, s), 1.15 (2H, q, J 7.5 Hz), 0.90 (3H, t, J 7.5 Hz), 0.82 (3H, t, J 7.5 Hz)

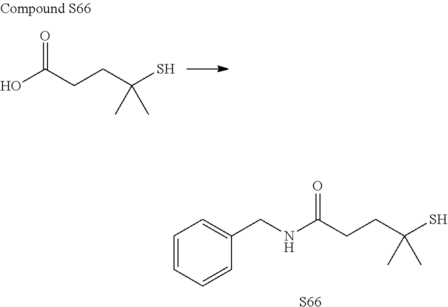

To a mixture of 2-methyl-2-mercaptopentanoic acid (0.74 g, 5.0 mmol) and acetic anhydride (0.52 mL, 5.5 mmol) in acetonitrile (10.0 mL) were added triethylamine (1.39 mL, 10.0 mmol) and DMAP (5 mg). The mixture was stirred for 1 hour, then benzylamine (1.37 mL, 12.5 mmol) was added to the mixture, and stirring was continued overnight. The volatiles were removed under vacuum to give a residue, which was subjected to flash silica gel column purification on an ISCO companion (ethyl acetate/hexane, 10-70%) to give 0.70 g (59%) of the title compound S66 as colorless oil. ¹H NMR (500 MHz, CDCl₃): δ7.36-7.32 (2H, m), 7.30-7.26 (3H, m), 5.73 (1H, s), 4.45 (2H, d, J 6.0 Hz), 2.43-2.38 (2H, m), 1.98-1.94 (2H, m), 1.39 (6H, s)

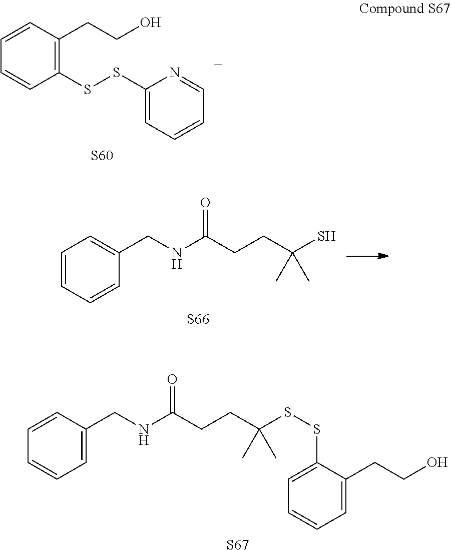 [SMILES: OCCc1ccccc1SSc1ccccn1 ; "S60" ; CC(C)(S)CCC(=O)NCc1ccccc1 ; "S66" ; CC(C)(CCC(=O)NCc1ccccc1)SSc1ccccc1CCO ; "S67"]

Compound S67 was prepared according to the procedure described for compound S41 using MeOTf activator as reported above. ¹H NMR (500 MHz, CDCl₃): δ7.81 (1H, d, J 8.0 Hz), 7.37-7.26 (3H, m), 7.21-7.15 (3H, m), 7.08-7.02 (2H, m), 5.14 (1H, s, br), 4.28 (2H, d, J 5.5 Hz), 3.89 (2H, t, J 6.5 Hz), 3.08 (2H, t, J 6.5 Hz), 2.12-2.05 (2H, m), 1.87-1.82 (2H, m), 1.29 (6H, s)

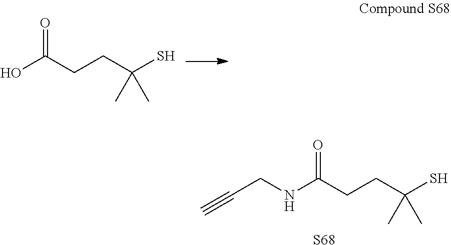

To a mixture of 2-methyl-2-mercaptopentanoic acid (0.74 g, 5.0 mmol) and acetic anhydride (0.52 mL, 5.5 mmol) in acetonitrile (10.0 mL) were added triethylamine (1.39 mL, 10.0 mmol) and DMAP (5 mg). The mixture was stirred for 1 hour, then propargylamine (0.69 g, 12.5 mmol) was added to the mixture, and stirring was continued for overnight. The volatiles were removed under vacuum to give a residue, which was subjected to flash silica gel column purification on an ISCO companion (ethyl acetate/hexane, 5-55%) to give 0.72 g (59%) of the title compound S68 as a white solid. ¹H NMR (500 MHz, CDCl₃): δ5.66 (1H, s), 4.06 (2H, dd, J 5.0, 2.5 Hz), 2.41-2.37 (2H, m), 2.23 (1H, t, J 2.5 Hz), 1.95-1.91 (2H, m), 1.39 (6H, s)

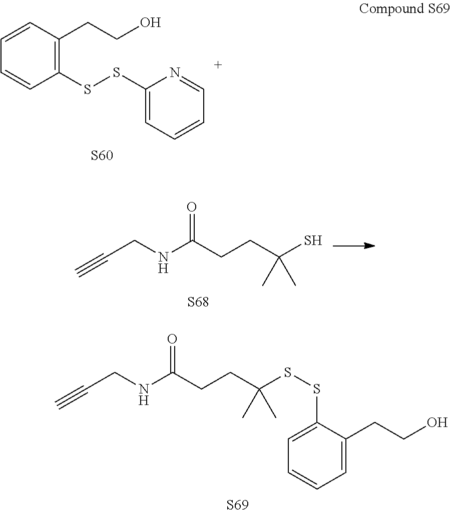 [SMILES: OCCc1ccccc1SSc1ccccn1 ; "S60" ; C#CCNC(=O)CCC(C)(C)S ; "S68" ; C#CCNC(=O)CCC(C)(C)SSc1ccccc1CCO ; "S69"]

Compound S69 was prepared according to the procedure described for compound S41 using MeOTf activator as reported above. ¹H NMR (500 MHz, CDCl₃): δ7.83 (1H, d, J 8.0 Hz), 7.30-7.16 (3H, m), 5.05 (1H, s), 3.95 (2H, t, J 6.5 Hz), 3.88 (2H, dd, J 5.5, 2.5 Hz), 3.15 (2H, t, J 6.5 Hz), 2.23 (1H, t, J 2.5 Hz), 2.10-2.04 (2H, m), 1.83-1.79 (2H, m), 1.28 (6H, s)

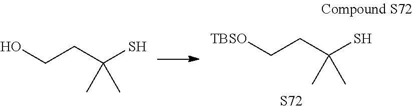

To a solution of 2-mercapto-2-methylbutan-1-ol (1.2 g, 10 mmol) in dichloromethane (25.0 mL) were added TBDMSCl (1.58 g, 10.5 mmol) and imidazole (1.02 g, 15 mmol) at 0° C. The resulting mixture was stirred for 30 min forming a large amount of white precipitate. The white solid was filtered and washed with 30.0 mL of dichloromethane. The filtrate was evaporated to give a residue, which was subjected to flash silica gel column purification on an ISCO companion (ethyl acetate/hexane 0-30%) to give 1.63 g (71%) of the title compound S72 as colorless oil. ¹H NMR (500 MHz, CDCl₃): δ7.83 (1H, d, J 8.0 Hz), 7.30-7.16 (3H, m), 5.05 (1H, s), 3.95 (2H, t, J 6.5 Hz), 3.88 (2H, dd, J 5.5, 2.5 Hz), 3.15 (2H, t, J 6.5 Hz), 2.23 (1H, t, J 2.5 Hz), 2.10-2.04 (2H, m), 1.83-1.79 (2H, m), 1.28 (6H, s)

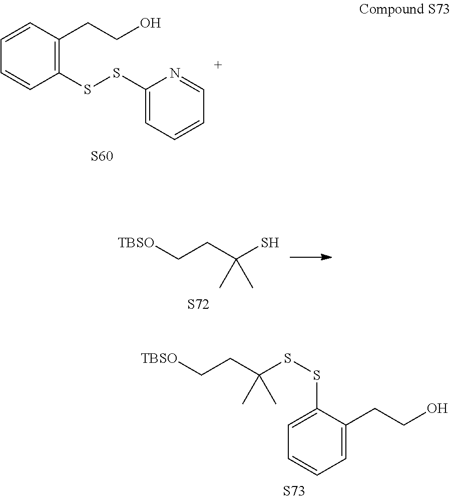 [SMILES: OCCc1ccccc1SSc1ccccn1 ; "S60" ; CC(C)(S)CCO[Si](C)(C)C(C)(C)C ; "S72" ; CC(C)(CCO[Si](C)(C)C(C)(C)C)SSc1ccccc1CCO ; "S73"]

Compound S73 was prepared according to the procedure described for compound S41 using MeOTf activator as reported above. ¹H NMR (500 MHz, CDCl₃): δ7.83 (1H, d, J 8.0 Hz), 7.30-7.12 (3H, m), 3.91 (2H, t, J 6.5 Hz), 3.68 (2H, t, J 7.0 Hz), 3.12 (2H, t, J 6.5 Hz), 1.83 (1H, t, J 6.5 Hz), 1.28 (6H, s), 0.87 (9H, s), 0.03 (6H, s)

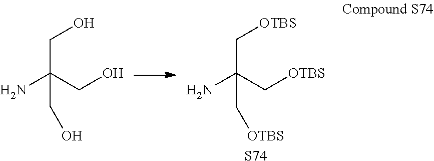 [SMILES: NC(CO)(CO)CO ; CC(C)(C)[Si](C)(C)OCC(N)(CO[Si](C)(C)C(C)(C)C)CO[Si](C)(C)C(C)(C)C ; "S74"]

To a solution of TBDMSCl (6.7 g, 44.6 mmol) and imidazole (6.3 g, 92.9 mmol) in DMF (5.0 mL) was added tris(hydroxymethyl)methylamine (1.5 g, 12.4 mmol) and stirred for 1 h. The mixture was diluted with water (15.0 mL), and extracted with dichloromethane (3×15.0 mL). The combined organic layers were dried over anhydrous sodium sulfate, and the filtrate was evaporated in vacuo to afford a residue, which was subjected to flash silica gel column purification on a ISCO companion (ethyl acetate/hexane, 0-20%) to give 4.0 g (70%) of S74 as colorless oil. ¹H NMR (500 MHz, CDCl₃): δ3.48 (6H, s), 0.89 (27H, s), 0.04 (18H, s)

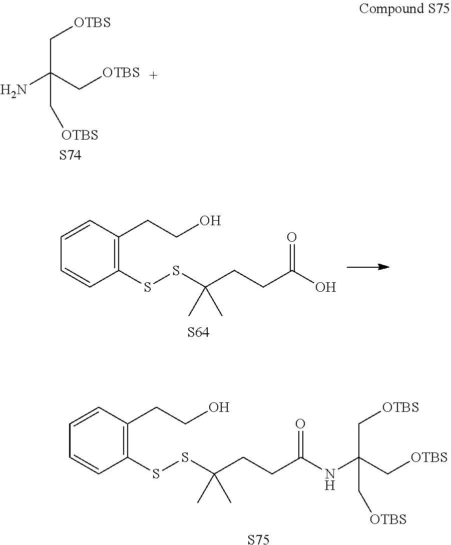 [SMILES: CC(C)(C)[Si](C)(C)OCC(N)(CO[Si](C)(C)C(C)(C)C)CO[Si](C)(C)C(C)(C)C ; "S74" ; CC(C)(CCC(=O)O)SSc1ccccc1CCO ; "S64" ; CC(C)(CCC(=O)NC(CO[Si](C)(C)C(C)(C)C)(CO[Si](C)(C)C(C)(C)C)CO[Si](C)(C)C(C)(C)C)SSc1ccccc1CCO ; "S75"]

To a mixture of compound S64 (0.6 g, 2.0 mmol) and S74 (1.16 g, 2.5 mmol) in DMF (10.0 mL) were added HATU (1.14 g, 3.0 mmol) and N,N-diisopropylethylamine (0.85 mL, 5 mmol). The mixture was stirred for 1 hour, at which time the volatiles were removed under high vacuum to give a residue, which was subjected to flash silica gel column purification on ISCO companion (ethyl acetate/hexane, 10-40%) to give 0.60 g (40%) of compound S75 as colorless oil. ¹H NMR (500 MHz, CDCl₃): δ7.81 (1H, d, J 8.0 Hz), 7.26-7.12 (3H, m), 5.45 (1H, s), 3.92 (2H, t, J 6.5 Hz), 3.80 (6H, s), 3.11 (2H, t, J 6.5 Hz), 2.14-2.10 (2H, m), 1.90-1.86 (2H, m), 1.23 (6H, s), 0.90 (27H, s), 0.04 (18H, s)

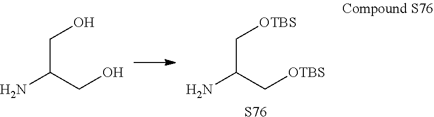 [SMILES: NC(CO)CO ; CC(C)(C)[Si](C)(C)OCC(N)CO[Si](C)(C)C(C)(C)C ; "S76"]

To a solution of TBDMSCl (7.2 g, 48 mmol), N,N-diisopropylethylamine (5.0 mL, 29 mmol) and DMAP (50 mg) in dichloromethane (50.0 mL) was added 2-amino-1,3-propan-diol (2.0 g, 22 mmol) and the mixture was stirred overnight. Volatiles were removed under high vacuum to give a residue, which was subjected to flash silica gel column purification on ISCO companion (ethyl acetate/hexane, 50-100% containing 2% triethylamine) to give 1.2 g (17%) of compound S76 as colorless oil. ¹H NMR (500 MHz, CDCl₃): δ3.70 (2H, dd, J 10.0, 5.5 Hz), 3.63 (2H, dd, J 10.0, 5.5 Hz), 3.04 (1H, m), 0.90 (18H, s), 0.07 (12H, s)

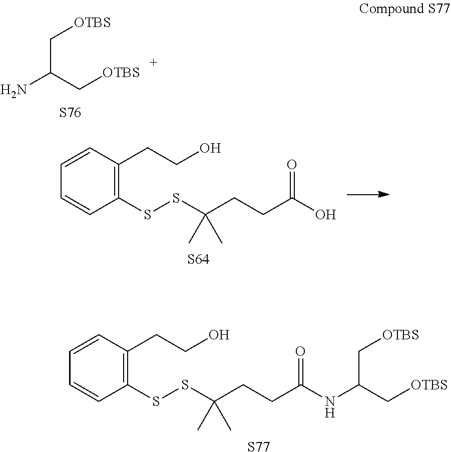 [SMILES: CC(C)(C)[Si](C)(C)OCC(N)CO[Si](C)(C)C(C)(C)C ; "S76" ; CC(C)(CCC(=O)O)SSc1ccccc1CCO ; "S64" ; CC(C)(CCC(=O)NC(CO[Si](C)(C)C(C)(C)C)CO[Si](C)(C)C(C)(C)C)SSc1ccccc1CCO ; "S77"]

To a mixture of compound S64 (0.77 g, 2.56 mmol) and S76 (0.82 g, 2.0 mmol) in DMF (10.0 mL) were added HATU (1.17 g, 3.07 mmol) and N,N-diisopropylethylamine (0.87 mL, 5.12 mmol). The mixture was stirred for 1 hour and the volatiles were removed under high vacuum to give a residue, which was subjected to flash silica gel column purification on ISCO companion (ethyl acetate/hexane, 10% -40%) to give 0.52 g (34%) of the title compound S77 as colorless oil. ¹H NMR (500 MHz, CDCl₃): δ7.81 (1H, d, J 7.5 Hz), 7.26-7.12 (3H, m), 5.59 (1H, d, J 8.5 Hz), 3.94 (2H, t, J 6.5 Hz), 3.92-3.82 (1H, m), 3.68 (2H, dd, J 13.5, 4.5 Hz), 3.50 (2H, dd, J 9.5, 6.5 Hz), 3.12 (2H, t, J 6.5 Hz), 2.16-2.10 (2H, m), 1.92-1.84 (2H, m), 1.26 (6H, s), 0.90 (18H, s), 0.07 (12H, s)

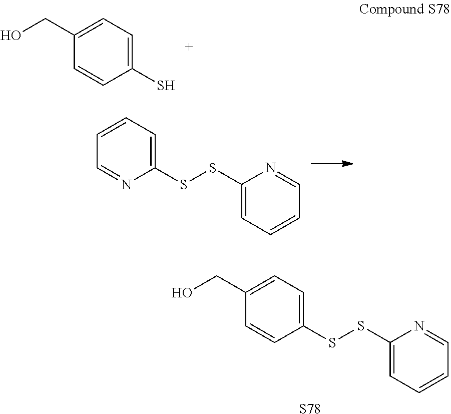

Compound S78 was prepared according to the procedure described for compound S55 using AcOH activator as reported above. ¹H NMR (500 MHz, CDCl₃): δ8.47 (1H, d, J 4.5 Hz), 7.70-7.60 (2H, m), 7.52 (2H, d, J 8.5 Hz), 7.31 (2H, d, J 8.5 Hz), 7.10 (1H, t, J 6.0 Hz), 4.67 (2H, s)

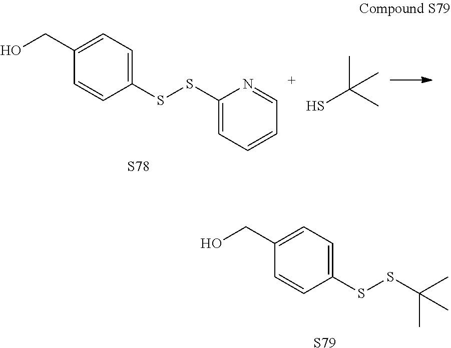

Compound S79 was prepared according to the procedure described for compound S41 using MeOTf activator as reported above. ¹H NMR (500 MHz, CDCl₃): δ7.55 (2H, d, J 8.0 Hz), 7.29 (2H, d, J 8.0 Hz), 4.67 (2H, s), 1.31 (9H, s)

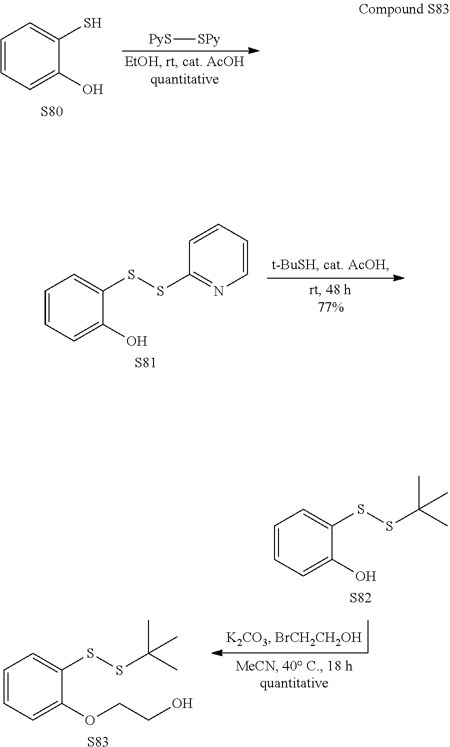

Compound S83 was prepared according to the procedure outlined in the above scheme.

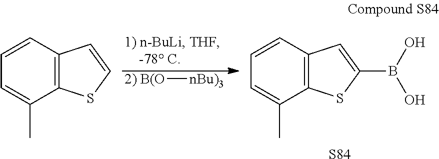

7-Methylbenzo[b]thiophene (0.74 g, 5 mmol) was dissolved in ether under argon, and the solution cooled to 0°. n-Butyl lithium (2.0 ml of 2.5M in hexane, 5 mmol) was added, while maintaining the temperature at 0-5° C. The mixture was stirred at 0° for 10 minutes, then for 45 minutes at room temperature. Then, the mixture was cooled to 0° and tributyl borate (1.47 ml, 5.5 mmol) was added dropwise. After stirring for 1 hour at 0°, the mixture was warmed to room temperature and allowed to stand overnight, at which time the reaction was quenched with 1M hydrochloric acid. The aqueous phase was extracted with ether and the ether layer was extracted with aqueous sodium hydroxide (1M). The basic aqueous layer was acidified with concentrated hydrochloric acid to pH 2 and extracted with ether (2×50 mL). The combined organic layers were dried over anhydrous Na₂SO₄. The solvent was evaporated in vacuo to give the crude S84 (0.80 g) as a white solid.

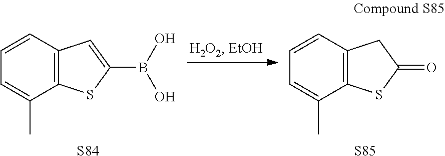

To a solution of crude S84 (0.80 g, 4.2 mmol) in EtOH (10.0 mL) was added hydrogen peroxide (30%, 1.4 mL) dropwise. After stirring overnight, the reaction mixture was carefully concentrated under reduced pressure, diluted with water (30 mL), and extracted with ethyl acetate (20 mL×3). The combined organic layer was dried over anhydrous sodium sulfate and concentrated in vacuo to afford a residue, which was subjected to flash silica gel column purification on an ISCO companion (ethyl acetate/hexane, 0-20%) to give 0.51 g (74%) of compound S85 as a colorless oil. ¹H NMR (500 MHz, CDCl₃): δ7.13 (3H, s), 4.00 (2H, s), 2.31 (3H, s)

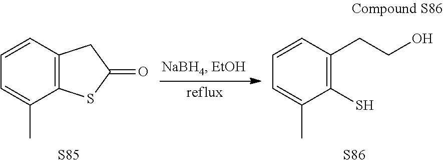

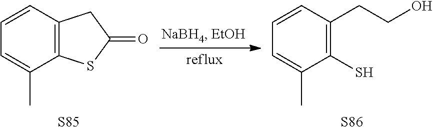

To a solution of S85 (0.51 g, 3.1 mmol) in EtOH (5 mL) was added NaBH₄ (0.59 g, 15.5 mmol) in one portion, and the mixture was refluxed for 15 min, and cooled to room temperature. Volatiles were evaporated to give a white slurry, which was dissolved in water and acidified to pH 2 with 1 M HCl. The mixture was extracted with dichloromethane (3×20 mL) and the combined organic layers were washed with brine, dried over anhydrous sodium sulfate, and concentrated in vacuo to afford crude compound S86 as colorless oil. ¹H NMR (500 MHz, CDCl₃): δ7.11-7.04 (3H, m), 3.92 (2H, t, J 6.5 Hz), 3.30 (1H, s), 3.05 (2H, t, J 6.5 Hz), 2.39 (3H, s)

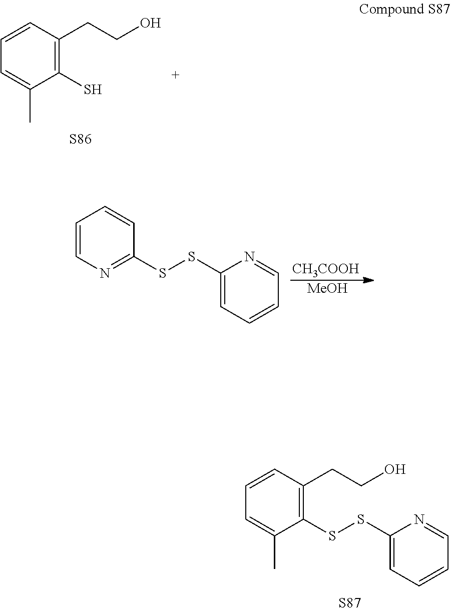

To a solution of dithiodipyridine (1.7 g, 7.8 mmol) and acetic acid (0.03 mL) in MeOH (10 mL) was added the crude S86 in MeOH (5 mL). The reaction mixture was stirred for 30 min and evaporated to give a yellow residue, which was subjected to purification by flash silica gel column chromatography on an ISCO companion (ethyl acetate/hexane, 0-40%) to give 0.38 g (44%) of compound S87 as colorless oil. ¹H NMR (500 MHz, CDCl₃): δ8.49 (1H, d, J 5.0 Hz), 7.64-7.58 (2H, m), 7.19 (1H, t, J 7.0 Hz), 7.13 (2H, t, J 6.5 Hz), 3.83 (2H, t, J 7.0 Hz), 3.26 (2H, t, J 6.5 Hz), 2.55 (3H, s)

### Compound S88

To a solution of compound S87 (0.57 g, 2.0 mmol) in 10.0 mL of dichloromethane was added MeOTf (0.36 g, 2.0 mmol) at room temperature. The reaction mixture stirred for 10 minutes, at which time tert-butylmercaptan (0.23 mL, 2.2 mmol) and diisopropylethylamine (0.5 mL) were added. The reaction mixture stirred for another 30 min at room temperature before being concentrated in vacuo. The crude mixture was purified using flash silica gel column purification on ISCO companion (ethyl acetate/hexane, 0-50%) to give compound S88 as colorless oil (0.46 g, 87%). ¹H NMR (500 MHz): δ7.17 (1H, t, J 7.0 Hz), 7.11 (m, 2H), 3.89 (2H, t, J 7.0 Hz), 3.34 (2H, t, J 7.0 Hz), 2.64 (3H, s), 1.27 (s, 9H)

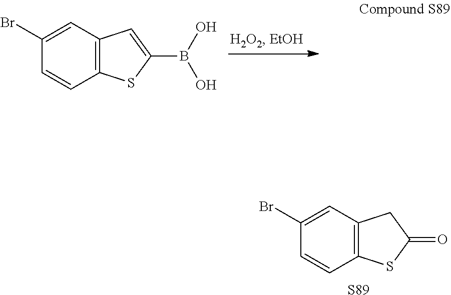 [SMILES: OB(O)c1cc2cc(Br)ccc2s1 ; O=C1Cc2cc(Br)ccc2S1 ; "S89"]

To a solution of 5-bromobenzo[b]thiophene-2-boronic acid (1.0 g, 3.90 mmol) in EtOH (12.0 mL) was added hydrogen peroxide (30%, 1.5 mL) dropwise. After stirring overnight, the reaction mixture was carefully concentrated under reduced pressure, diluted with water (30 mL), and extracted with ethyl acetate (20 mL×3). The combined organic layer was dried over anhydrous sodium sulfate and concentrated in vacuo to afford a residue, which was subjected to flash silica gel column purification on an ISCO companion (ethyl acetate/hexane, 0-20%) to give 0.64 g (72%) of compound S89 as colorless oil. ¹H NMR (500 MHz, CDCl₃): δ7.44 (1H, s), 7.43 (1H, d, J 8.0 Hz), 7.21 (1H, d, J 8.0 Hz), 3.96 (2H, s)

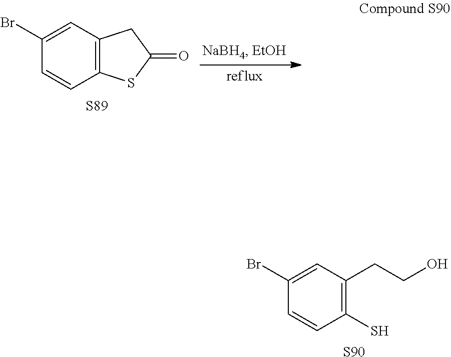 [SMILES: O=C1Cc2cc(Br)ccc2S1 ; "S89" ; OCCc1cc(Br)ccc1S ; "S90"]

To a refluxing solution of S89 (0.64 g, 2.8 mmol) in EtOH (10 mL) was added NaBH₄ (0.53 g, 13.9 mmol) in one portion. The reaction mixture was refluxed for another 15 min and cooled to room temperature, volatiles were evaporated to give white slurry, which was dissolved in water, and the solution was acidified to pH 2 with 1 M HCl. The water layer was extracted with dichloromethane (3×20 mL), and the combined organic layers were washed with brine, dried over anhydrous sodium sulfate, and concentrated in vacuo to afford the crude compound S90 as a white solid. ¹H NMR (500 MHz, CDCl₃): δ7.37 (1H, s), 7.23 (1H, d, J 8.0 Hz), 7.18 (1H, d, J 8.0 Hz), 3.90 (2H, t, J 6.5 Hz), 3.42 (1H, s), 2.94 (2H, t, J 6.5 Hz)

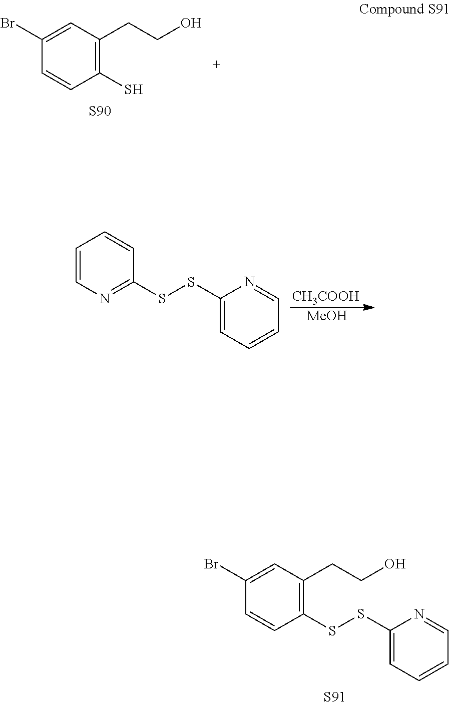

To a solution of dithiodipyridine (1.84 g, 8.34 mmol) and acetic acid (0.03 mL) in MeOH (10 mL) was added the crude S90 in MeOH (5 mL) and the mixture was stirred for 30 min then evaporated to give a yellow residue, This was subjected to flash silica gel column purification on a ISCO companion (ethyl acetate/hexane, 0-40%) to give 0.50 g (53% for two steps) of the compound S91 as colorless oil. ¹H NMR (500 MHz, CDCl₃): δ8.47 (1H, d, J 5.0 Hz), 7.64-7.58 (3H, m), 7.31-7.26 (2H, m), 7.13 (1H, m), 3.95 (2H, t, J 6.5 Hz), 3.12 (2H, t, J 6.5 Hz)

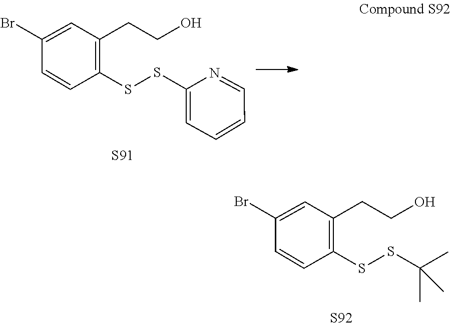 [SMILES: OCCc1cc(Br)ccc1SSc1ccccn1 ; "S91" ; CC(C)(C)SSc1ccc(Br)cc1CCO ; "S92"]

To a solution of compound S91 (0.50 g, 1.47 mmol) in 10.0 mL of dichloromethane was added MeOTf (0.24 g, 1.47 mmol) at room temperature. The reaction mixture was stirred for 10 minutes, at which time tert-butylmercaptan (0.18 mL, 1.62 mmol) and N,N-diisopropylethylamine (0.5 mL) were added. The reaction mixture was stirred for another 30 min at room temperature and concentrated in vacuo. The crude mixture was purified using flash silica gel column purification on an ISCO companion (ethyl acetate/hexane solvent, 0-50%) to give compound S92 as colorless oil (0.37 g, 78%). ¹H NMR (500 MHz): δ7.72 (2H, d, J 8.5 Hz), 7.34 (2H, m), 3.91 (2H, t, J 7.0 Hz), 3.07 (2H, t, J 7.0 Hz), 1.29 (s, 9H)

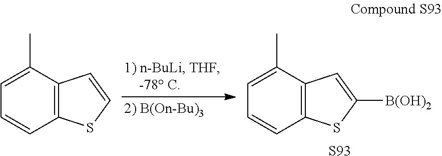

4-Methylbenzothiophene (1.0 g, 6.75 mmol) was dissolved in ether under argon and the solution was cooled to 0° C. n-Butyllithium (2.7 mL of 2.5M in hexane, 6.75 mmol) was added while maintaining the temperature at 0-5° C. The mixture was stirred at 0° C. for 10 minutes, then 45 minutes at room temperature, cooled again to 0° C., and tributyl borate (1.99 mL, 7.43 mmol) was added dropwise. The reaction mixture was stirred for 1 hour at 0° C., then warmed to room temperature, and allowed to stand overnight followed by quenching with 1M hydrochloric acid. The aqueous phase extracted with ether (2×30 mL), and the combined organic layers were washed with aqueous sodium hydroxide (1M). The aqueous basic layer was acidified with concentrated hydrochloric acid to pH 2 and extracted with ether (2×30 mL). The combined organic layers were dried over anhydrous Na₂SO₄. The solvent was evaporated in vacuo to give the crude S93 (1.05 g, 81%) as a white solid, which was used directly in the next step without further purification. ¹H NMR (500 MHz, CD₃OD): δ7.93 (1H, s), 7.70 (1H, d, J 8.0 Hz), 7.25 (1H, t, J 7.0 Hz), 7.13 (1H, d, J 7.0 Hz), 7.04 (1H, d, J 7.0 Hz), 2.62 (3H, s)

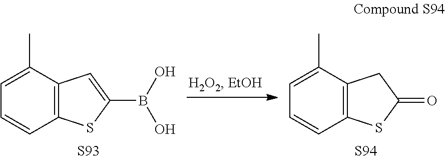

To a solution of crude S93 (1.05 g, 5.5 mmol) in EtOH (10.0 mL) was added hydrogen peroxide (30%, 1.0 mL) drop wise. After stirring overnight, the reaction mixture was carefully concentrated under reduced pressure, diluted with water (30 mL), and extracted with ethyl acetate (3×20 mL). The combined organic layers were dried over anhydrous sodium sulfate and concentrated in vacuo to afford a residue, which was subjected to flash silica gel column purification on a ISCO companion (ethyl acetate/hexane=5-15%) to give 0.80 g (89%) of the title compound S94 as a colorless oil. ¹H NMR (500 MHz, CDCl₃): δ7.23-7.17 (2H, m), 7.04 (1H, d, J 7.0 Hz), 3.85 (2H, s), 2.28 (3H, s).

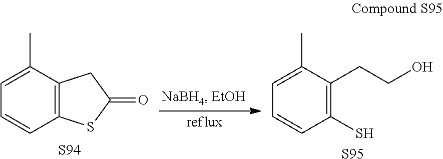

To a refluxing solution of S94 (0.69 g, 4.2 mmol) in EtOH (25 mL) was added NaBH₄ (0.79 g, 21 mmol) in one portion. The mixture was refluxed for another 15 min, then cooled to room temperature. The mixture was evaporated to give white slurry, which was dissolved in water. The mixture was acidified to pH 2 with 1 M HCl. The mixture was extracted with dichloromethane (3×20 mL). The combined organic layers were washed with brine, dried over anhydrous sodium sulfate, and concentrated in vacuo to afford a residue, which was subjected to flash silica gel column purification on an ISCO companion (ethyl acetate/hexane=0-40%) to give 0.67 g (95%) of the title compound S95 as a colorless oil. ¹H NMR (500 MHz, CDCl₃): δ7.16 (1H, m), 7.00-6.96 (2H, m), 3.86 (2H, t, J 7.0 Hz), 3.44 (1H, s), 3.06 (2H, t, J 7.0 Hz), 2.35 (3H, s)

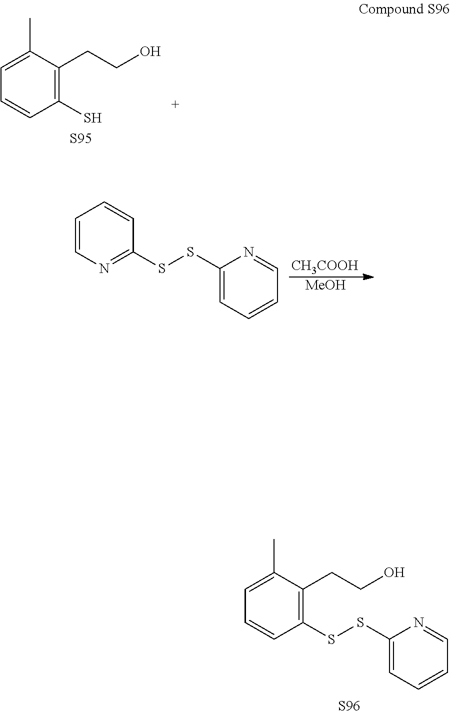

To a solution of dithiodipyridine (2.64 g, 12.0 mmol) and acetic acid (0.1 mL) in MeOH (60 mL) was added the solution of S95 (0.66 g, 3.94 mmol) in MeOH (5 mL). The mixture was stirred for 30 min, and evaporated to give a yellow residue, which was subjected to flash silica gel column purification on a ISCO companion (ethyl acetate/hexane=0-40%) to give 1.09 g (100%) of the title compound S96 as colorless oil. ¹H NMR (500 MHz, CDCl₃): δ8.49 (1H, d, J 4.5 Hz), 7.64-7.58 (2H, m), 7.50 (1H, dd, J 7.0, 2.5 Hz), 7.11 (1H, m), 7.08-7.02 (2H, m), 3.91 (2H, t, J 7.0 Hz), 3.25 (2H, t, J 7.0 Hz), 2.38 (3H, s)

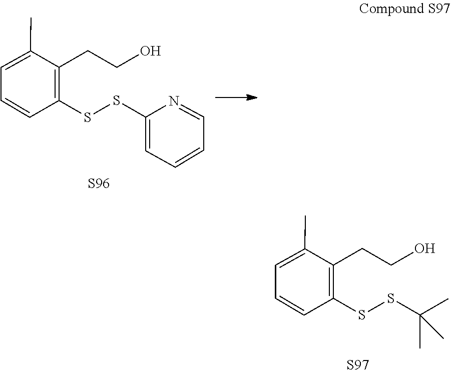 [SMILES: Cc1cccc(SSc2ccccn2)c1CCO ; "S96" ; Cc1cccc(SSC(C)(C)C)c1CCO ; "S97"]

To a solution of compound S96 (0.69 g, 2.5 mmol) in 10.0 mL of dichloromethane was added MeOTf (0.41 g, 2.5 mmol) at room temperature. The reaction mixture was stirred for 10 minutes, at which time tert-butylmercaptan (0.34 mL, 3.0 mmol) and diisopropylethylamine (0.5 mL) were added, and stirring was continued for another 30 min at room temperature. The resulting mixture was concentrated in vacuo. The crude mixture was purified using flash silica gel column purification on ISCO companion (ethyl acetate/hexane solvent=0-40%) to give compound S97 as colorless oil (0.45 g, 70%). ¹H NMR (500 MHz): δ7.71 (1H, d, J 8.0 Hz), 7.12 (1H, t, J 8.0 Hz), 7.01 (1H, d, J 8.0 Hz), 3.86 (2H, t, J 7.0 Hz), 3.21 (2H, t, J 7.0 Hz), 2.37 (3H, s), 1.30 (s, 9H)

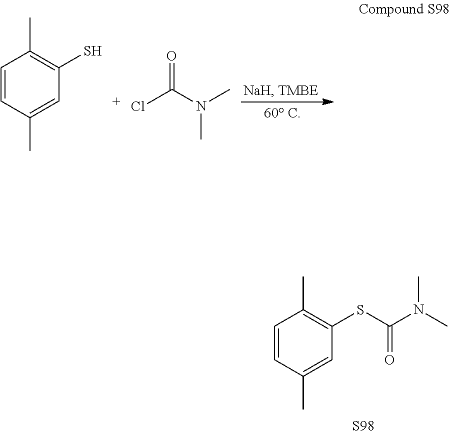

Sodium hydride (60% in oil) (1.80 g, 45.0 mmol) and t-butyl methyl ether (15 mL) were added to a round bottom flask under an argon atmosphere at 0° C. To the mixture was added a solution of 2,5-dimethylbenzenethiol (4.07 mL, 30.0 mmol) in t-butyl methyl ether (15 mL) dropwise followed by addition of a solution of dimethylcarbamoyl chloride (3.03 mL, 33.0 mmol) in t-butyl methyl ether (10 mL). The reaction mixture was heated to 60° C., stirred for 1.5 hours, and disappearance of the starting materials was confirmed. The mixture was cooled in an ice bath and neutralized with 1M hydrochloric acid (20 mL). The aqueous layer was extracted with ether (2×30 mL), and the organic layers were combined and washed with aqueous 1M sodium hydroxide, water, and brine. After drying the organic layer over anhydrous sodium sulfate, the filtrate was evaporated to give a residue, which was subjected to flash silica gel column purification on an ISCO companion (ethyl acetate/hexane=5-50%) to give the title compound S98 as a colorless oil (5.15 g, 82%). ¹H NMR (500 MHz, CDCl₃): δ7.30 (1H, s), 7.18 (1H, d, J 8.0 Hz), 7.11 (1H, d, J 8.0 Hz), 3.15-3.00 (6H, br s), 2.36 (3H, s), 2.30 (3H, s)

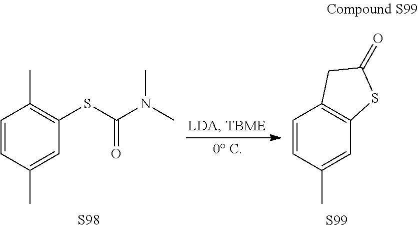

To a solution of LDA (12.5 mL, 2M in THF, 25 mmol) in t-butyl methyl ether (35 mL) was added a solution of a dimethyl-thiocarbamic acid S-(2,3-dimethylphenyl) ester (S98, 2.09 g, 10 mmol) in t-butyl methyl ether (8 mL) dropwise at 0° C. and the resulting mixture was stirred at 0° C. for 30 minutes. The reaction mixture was quenched by addition of 6 mL of acetic acid followed by addition of 2 mL of 37% aqueous HCl solution and water, and the temperature was raised to near room temperature, and the phases were separated. The aqueous layer was extracted with ethyl acetate (2×50 mL), and the organic layers were combined and washed with brine. After drying the organic layer over magnesium sulfate, the filtrate was concentrated under reduced pressure to give a residue, which was subjected to flash silica gel column purification on an ISCO companion (ethyl acetate/hexane=5-25%) to give the title compound S99 as a white solid (0.98 g, 60%). ¹H NMR (500 MHz, CDCl₃): δ7.16 (2H, s), 7.01 (1H, d, J 8.0 Hz), 3.92 (2H, s), 2.36 (3H, s)

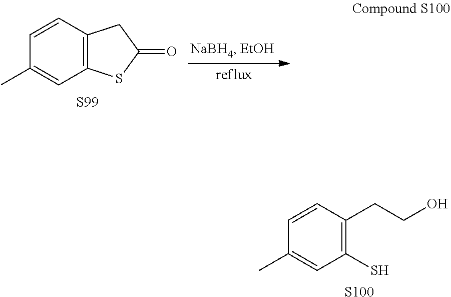

To a refluxing solution of S99 (0.98 g, 6.0 mmol) in EtOH (30 mL) was added NaBH₄ (1.13 g, 30 mmol) in one portion. The mixture was refluxed for another 15 min and cooled to room temperature. The mixture was evaporated to give white slurry, which was dissolved in water and acidified to pH 2 with 1M HCl. The mixture was extracted with dichloromethane (3×20 mL). The combined organic layers were washed with brine, dried over anhydrous sodium sulfate, and concentrated in vacuo to afford the crude title compound S100 as colorless oil. ¹H NMR (500 MHz, CDCl₃): δ7.14 (1H, s), 7.08 (1H, d, J 8.0 Hz), 6.94 (1H, d, J 8.0 Hz), 3.88 (2H, t, J 6.5 Hz), 3.36 (1H, s), 2.94 (2H, t, J 6.5 Hz), 2.28 (3H, s)

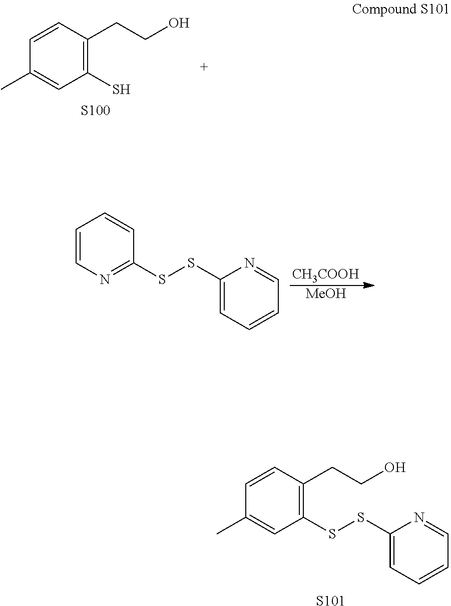

To a solution of dithiodipyridine (4.0 g, 18 mmol) and acetic acid (0.1 mL) in MeOH (70 mL) was added compound S100 in MeOH (10 mL). The reaction mixture was stirred for 30 min, evaporated to give a yellow residue, which was subjected to flash silica gel column purification on an ISCO companion (ethyl acetate/hexane=0-40%) to give 1.55 g (93% in two steps) of the title compound S101 as a colorless oil. ¹H NMR (500 MHz, CDCl₃): δ8.49 (1H, d, J 4.5 Hz), 7.65-7.61 (2H, m), 7.45 (1H, s), 7.13-7.11 (2H, m), 7.01 (1H, d, J 8.0 Hz), 3.92 (2H, t, J 6.5 Hz), 3.13 (2H, t, J 6.5 Hz), 2.25 (3H, s)

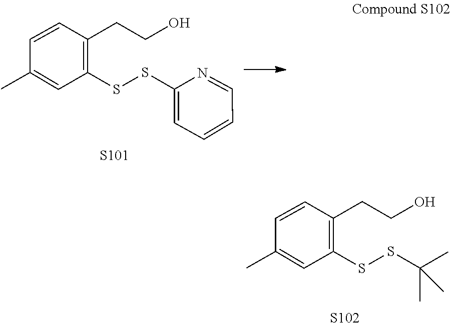 [SMILES: Cc1ccc(CCO)c(SSc2ccccn2)c1 ; "S101" ; Cc1ccc(CCO)c(SSC(C)(C)C)c1 ; "S102"]

To a solution of compound S101 (0.69 g, 2.5 mmol) in 10.0 mL of dichloromethane was added MeOTf (0.41 g, 2.5 mmol) at room temperature. The reaction mixture was stirred for 10 minutes, at which time tert-butylmercaptan (0.34 mL, 3.0 mmol) and N,N-diisopropylethylamine (0.5 mL) were added, and stirring was continued for another 30 min at room temperature. The resulting mixture was concentrated in vacuo. The crude mixture was purified using flash silica gel column purification on ISCO companion (ethyl acetate/hexane solvent=0-40%) to give compound S102 as colorless oil (0.49 g, 77%). ¹H NMR (500 MHz): δ7.64 (1H, s), 7.06 (1H, d, J 8.0 Hz), 6.95 (1H, d, J 8.0 Hz), 3.89 (2H, t, J 7.0 Hz), 3.08 (2H, t, J 7.0 Hz), 2.36 (3H, s), 1.30 (s, 9H).

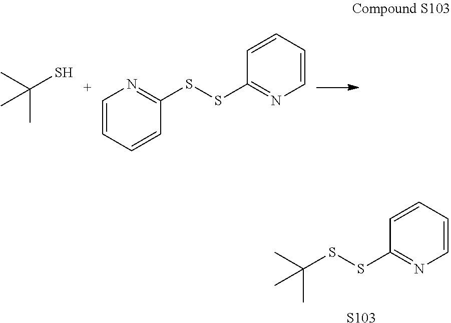

To a solution of tert-butylmercaptan (4.5 g, 50 mmol) in ethanol (150 mL) were added dithiodipyridine (12.1 g, 55.0 mmol) and acetic acid (3.5 mL). The mixture was stirred overnight, evaporated to give a residue, which was then dissolved in 100 mL of ethyl acetate. The solution was washed with 1N NaOH (50 mL×3) and brine. The organic layer was dried over anhydrous Na₂SO₄, filtered, and evaporated to give a residue, which was subjected to flash silica gel column purification on an ISCO companion (ethyl acetate/hexane, 5-20%) to give 7.3 g (73%) of the title compound S103 as colorless oil. ¹H NMR (500 MHz, CDCl₃): δ8.44 (1H, d, J 5.0 Hz), 7.75 (1H, d, J 8.0 Hz), 7.63 (1H, td, J 8.0, 1.5 Hz), 7.06 (1H, m), 1.33 (9H, s)

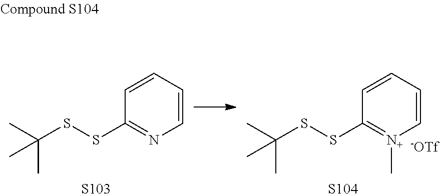

To a solution of S103 (1.81 g, 9.0 mmol) in hexane (30 mL) was added MeOTf (1.48 g, 9.0 mmol). The mixture was stirred for 15 min, the resulting precipitate was filtered and washed with hexane (10 mL×3). The isolated, white solid was dried under vacuum to give crude S104, which was used in the next reaction without further purification.

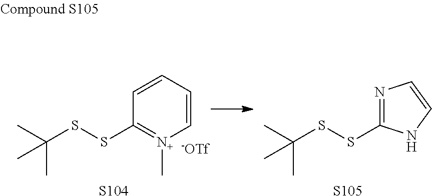

To a solution of S104 (9.0 mmol) in DMF (5 mL) was added 2-mercaptoimidazole (0.90 g, 9.0 mmol) to form a yellow mixture. The mixture was stirred for 30 min, at which time diisopropylethylamine (1 mL) and water (4 mL) were added. Upon addition of water (20 mL), a precipitate formed, which was filtered, washed, with water followed by hexane, and dried under vacuum to give 1.13 g (67% in 2 steps) of S105 as a white solid. ¹H NMR (500 MHz, CDCl₃): δ7.11 (1H, s), 1.33 (9H, s)

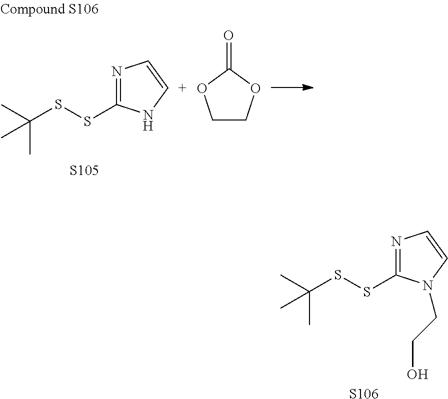

To a suspension of NaH (0.38 g, 60% in mineral oil, 9.4 mmol) in THE (5 mL) under argon at 0° C. was added S105 (0.89 g, 4.7 mmol) in THE (2 mL). The resulting mixture was warmed to room temperature and stirred for 1 h. The reaction mixture was cooled to 0° C., a solution of ethylene carbonate (0.50 g, 5.6 mmol) in THE (3 mL) was added, and the resulting mixture was warmed to room temperature and stirred overnight. Saturated, aqueous NH₄Cl solution was added to quench the reaction, and the resulting mixture was extracted with ethyl acetate (20 mL×3). The combined organic layers were washed with brine and dried over anhydrous Na₂SO₄, filtered, and evaporated to give a residue, which was purified by flash silica gel column using an ISCO companion (ethyl acetate/dichloromethane, 10-100%) to give 0.39 g (35%) of the compound S106 as a white solid. ¹H NMR (500 MHz, CDCl₃): δ7.16 (1H, d, J 1.0 Hz), 7.06 (1H, d, J 1.0 Hz), 4.28 (1H, t, J 5.0 Hz), 4.00 (1H, t, J 5.0 Hz), 1.36 (9H, s)

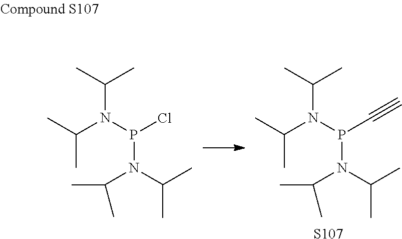 [SMILES: CC(C)N(C(C)C)P(Cl)N(C(C)C)C(C)C ; C#CP(N(C(C)C)C(C)C)N(C(C)C)C(C)C ; "S107"]

To a flame-dried 500-mL Schlenk flask equipped with a magnetic stir bar and septum under nitrogen was added bis(N,N-diisopropylamino)chlorophosphine (2.66 g, 10 mmol), anhydrous diethyl ether (200 mL) and the mixture was cooled to 0° C. To this solution, ethynylmagnesium bromide (0.5 M in THF, 11 mmol) was added drop-wise via a syringe over a period of 15 min, and the reaction mixture was allowed to stir at 0° C. for one hour. The mixture was allowed to attain room temperature, filtered under nitrogen, and the solution was concentrated on a rotary evaporator. The resulting viscous oil extracted three times with anhydrous hexanes during which the oil transformed into a solid. The solid was then dissolved in a minimum volume of anhydrous acetonitrile, and the resulting solution was extracted twice with anhydrous hexanes. The hexane fractions were combined and concentrated in vacuum to give a translucent white oil S107 (2.3 g, 90%), which was used without further purification.

### Preparation of Benzimidazoles Linked to Disulfide Linkages

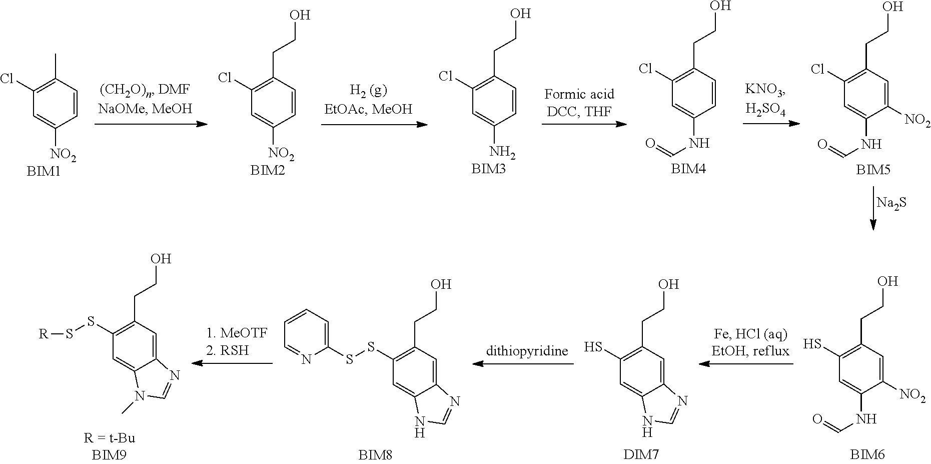

### Preparation of N-Methyl 1-Hydroxyethyl 2-Mercapto 4, 5-Benzimidazole Linker (BIM9):

Commercially available 2-chloro-4-nitro-toluene (BIM1) can be homologated with paraformaldehyde under basic conditions to provide phenethylalcohol (BIM2). Other bases can include but are not-limited to NaOEt, KOtBu, DIEA, TEA, DBU, and inorganic bases. Hydrogenation of the 4-nitro group and formylation can afford BIM4. After nitration of BIM4 to BIM5, a thiol group can be introduced through treatment with Na₂S to give mercaptan (BIM6). Reduction of the 5-nitro through a reduced iron catalyst with heating can concomitantly afford 2-mercapto benzimidazole (BIM7). After conversion to the thiopyridine (BIM8), activation with MeOTf and treatment with t-butyl mercaptan (R=HS-tBu) can yield (BIM9).

### Preparation of PEG Chains Linked to Disulfide Linkages

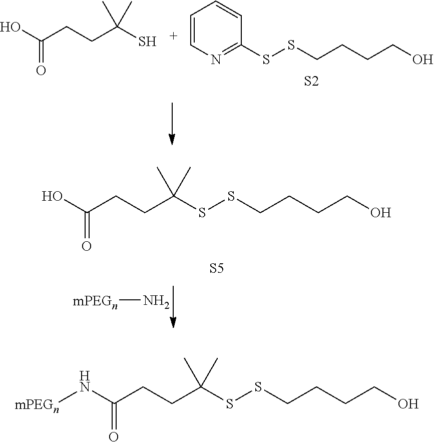

General Procedure for the Synthesis of Disulfide PEG Side Chains:

To a solution of carboxylic acid S5 (1.98 mmol) and mPEGₙ-NH₂ (1.98 mmol) in anhydrous dimethylformamide (5.0 mL) at room temperature were added sequentially HATU (2.97 mmol) and N,N-diisopropylethylamine (2.97 mmol) in that order, and the resulting mixture was stirred for 2 hours. TLC showed completion of reaction. Dimethylformamide was removed under vacuum, and the residue was dissolved in CH₂Cl₂ (10.0 mL). The mixture was washed with brine (10 mL×2), and the organic layer was dried over anhydrous Na₂SO₄, and evaporated to give crude compound. Silica gel column purification using an ISCO companion (methanol/methylene chloride, 0-10%) gave the compound as thick syrup.

### Phosphoramidites and Other Monomers

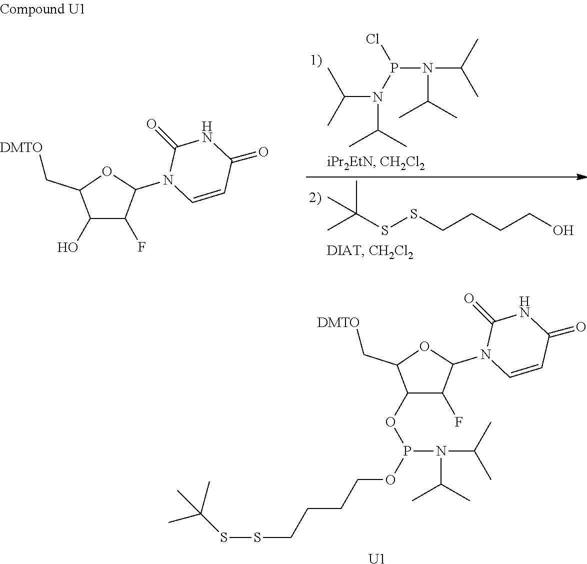

To a −78° C. cooled solution of 5′-O-(4,4′-dimethoxytrityl)-2′-F-uridine (3.9 g, 5.6 mmol) and N,N-diisopropylethylamine (1.1 mL, 6.16 mmol) in 25.0 mL of dry dichloromethane was added dropwise a solution of bis-(N,N-diisopropylamino)-chlorophosphine (1.64 g, 6.16 mmol) in 5.0 mL of dichloromethane under Argon atmosphere. The reaction mixture was allowed to warm to room temperature while stirring was maintained for 1 hour. A solution of S8 (1.0 g, 5.6 mmol) in 5.0 mL of dry dichloromethane was added dropwise and stirred for 10 minutes before a suspension of diisoproprylammonium tetrazolide (DIAT) (1.0 g, 5.88 mmol) in 5.0 mL of dichloromethane was added portion wise. The reaction mixture was further stirred for 16 hours at room temperature. The crude mixture was diluted with 200 mL of dichloromethane and washed sequentially with saturated NaHCO₃ solution (50 mL) and brine (50 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 2.32 g (48%) of product U1 (diastereomeric mixture) as white powder. ESI MS for C₄₄H₅₉FN₃O₈PS₂ Calculated 872.05, Observed 871.0 [M−H]⁺. ³¹P NMR (202 MHz, CDCl₃): δ150.7 (d, J 7.5 Hz), 150.0 (d, J 9.3 Hz).

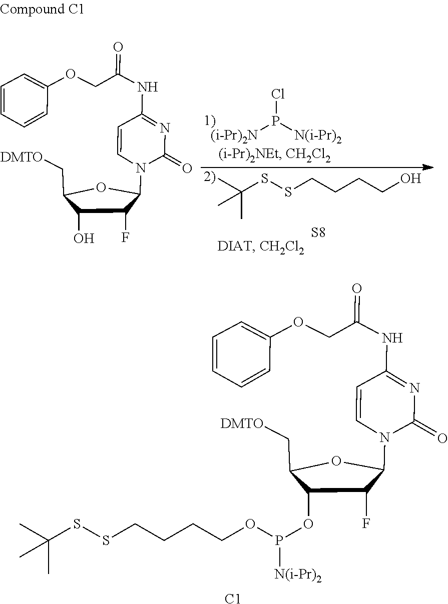

To a −78° C. cooled solution of 5′-O-(4,4′-dimethoxytrityl)-2′-F-cytidine (n-PAC) (3.8 g, 5.6 mmol) and N,N-diisopropylethylamine (1.1 mL, 6.16 mmol) in 25.0 mL of dry dichloromethane was added dropwise a solution of bis-(N,N-diisopropylamino)-chlorophosphine (1.64 g, 6.16 mmol) in 5.0 mL of dichloromethane under argon atmosphere. The reaction mixture was allowed to warm to room temperature while stirring was maintained (1 hour). A solution of S8 (1.0 g, 5.6 mmol) in 5.0 mL of dry dichloromethane was added dropwise and stirred for 10 minutes before a suspension of diisoproprylammonium tetrazolide (1.0 g, 5.88 mmol) in 5.0 mL of dichloromethane was added portion wise. The reaction mixture was further stirred for 16 hours at room temperature. The crude mixture was diluted with 200 mL of dichloromethane and washed sequentially with saturated NaHCO₃ solution (50 mL) and brine (50 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 1.43 g (26%) of product C₁ (diastereomeric mixture) as white powder. ESI MS for C₅₂H₆₆FN₄O₉PS₂ Calculated 1005.2, Observed 1004.0 [M−H]⁺. ³¹P NMR (202 MHz, CDCl₃): δ150.6 (d, J 6.5 Hz), 150.0 (d, J 5.5 Hz).

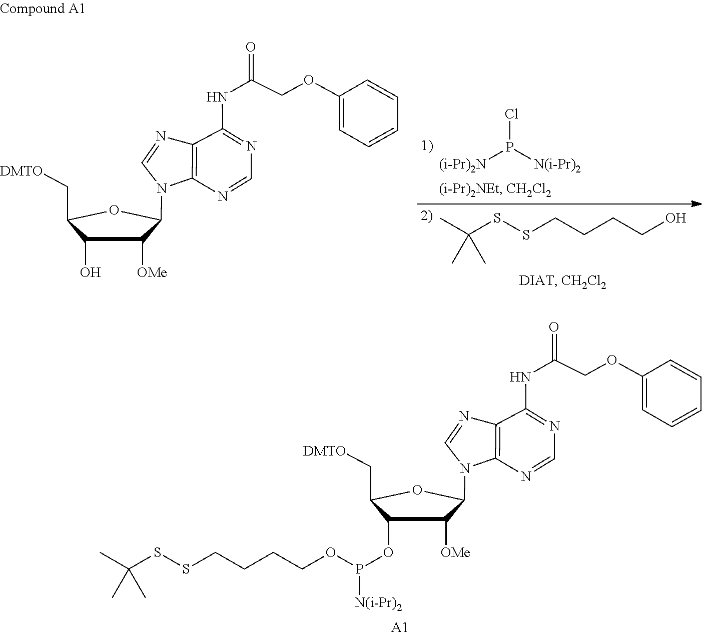

To a −78° C. cooled solution of 5′-O-(4,4′-dimethoxytrityl)-2′-O-methyl-adenosine (n-PAC) (4.02 g, 5.6 mmol) and N,N-diisopropylethylamine (1.1 mL, 6.16 mmol) in 25.0 mL of dry dichloromethane was added dropwise a solution of bis-(N,N-diisopropylamino)chlorophosphine (1.64 g, 6.16 mmol) in 5.0 mL of dichloromethane under argon atmosphere. The reaction mixture was allowed to warm to room temperature, while stirring was maintained (1 hour). A solution of S8 (1.0 g, 5.6 mmol) in 5.0 mL of dry dichloromethane was added dropwise, and the reaction mixture was stirred for 10 minutes before a suspension of diisoproprylammonium tetrazolide (1.0 g, 5.88 mmol) in 5.0 mL of dichloromethane was added portionwise. The reaction mixture was further stirred for 16 hours at room temperature. The crude mixture was diluted with 200 mL of dichloromethane and washed sequentially with saturated NaHCO₃ solution (50 mL) and brine (50 mL), then dried over anhydrous Na₂SO₄. The solvent was evaporated in vacuo and the crude mixture was purified by silica gel column chromatography using ethyl acetate/hexane solvent system (0-30% gradient on Combi Flash Rf Instrument) to give 1.99 g (35%) of product A1 (diastereomeric mixture) as white powder. ESI MS for C₅₄H₆₉N₆O₉PS₂ Calculated 1041.26, Observed 1040.4 [M−H]⁺. ³¹P NMR (202 MHz, CDCl₃): δ150.4, 149.5.

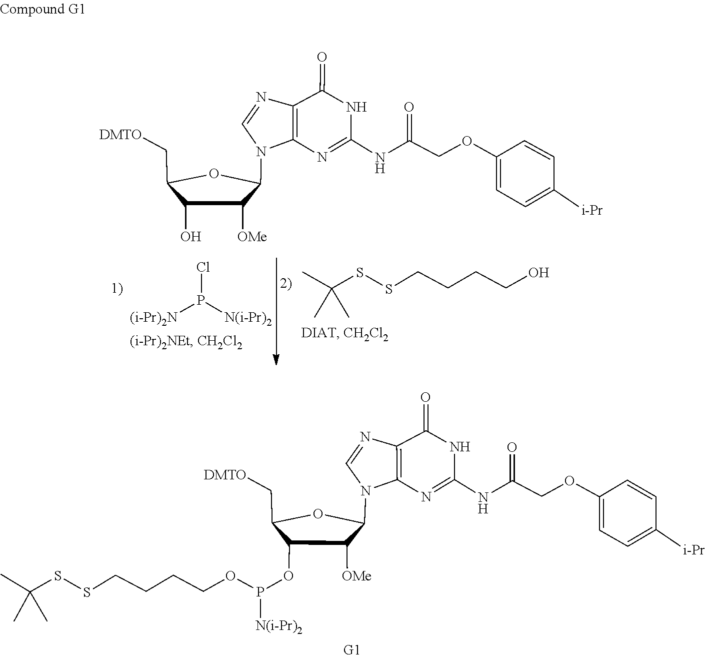

To a −78° C. cooled solution of 5′-O-(4,4′-dimethoxytrityl)-2′-O-methyl-guanosine (n-isopropyl-PAC) (3.2 g, 4.1 mmol) and N,N-diisopropylethylamine (0.78 mL, 4.5 mmol) in 20.0 mL of dry dichloromethane was added dropwise a solution of bis-(N,N-diisopropylamino) chlorophosphine (1.2 g, 4.5 mmol) in 5.0 mL of dichloromethane under argon atmosphere. The reaction mixture was allowed to warm to room temperature while stirring was maintained (1 hour). A solution of S8 (0.74 g, 4.1 mmol) in 5.0 mL of dry dichloromethane was added dropwise, and the resulting mixture was stirred for 10 minutes, at which time a suspension of diisoproprylammonium tetrazolide (0.74 g, 4.3 mmol) in 5.0 mL of dichloromethane was added portionwise. The reaction mixture was further stirred for 16 hours at room temperature. The crude mixture was diluted with 100 mL of dichloromethane, washed sequentially by saturated NaHCO₃ solution (25 mL) and brine (25 mL), and dried over anhydrous Na₂SO₄. The solvent was evaporated in vacuo, and the crude mixture was purified by silica gel column chromatography using ethyl acetate/hexane solvent system (0-100% gradient on Combi Flash Rf Instrument) to give 0.60 g (13%) of product G1 (diastereomeric mixture) as white powder. ESI MS for C₅₇H₇₅N₆O₁₀PS₂ Calculated 1099.34, Observed 1098.2[M]⁺. ³¹P NMR (202 MHz, CDCl₃): δ150.5, 149.9.

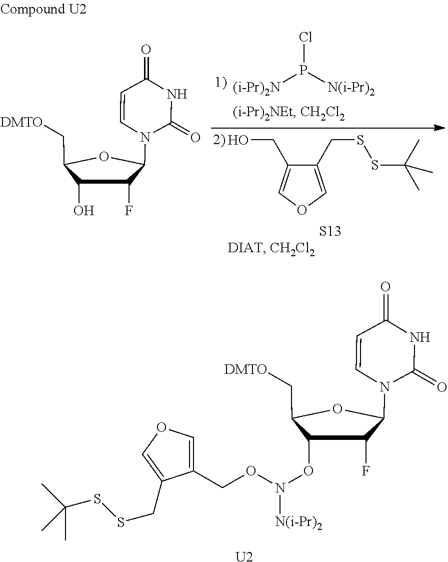

To a −78° C. cooled solution of 5′-O-(4,4′-dimethoxytrityl)-2′-F-uridine (0.36 g, 0.65 mmol) and N,N-diisopropylethylamine (0.13 mL, 0.72 mmol) in 10.0 mL of dry dichloromethane was added dropwise a solution of bis-(N,N-diisopropylamino)-chlorophosphine (0.19 g, 0.72 mmol) in 3.0 mL of dichloromethane under argon atmosphere. The reaction mixture was allowed to warm to room temperature, while stirring was maintained (1 hour). A solution of S13 (0.15 g, 0.65 mmol) in 3.0 mL of dry dichloromethane was added dropwise, and the resulting mixture was stirred for 10 minutes, at which time a suspension of diisoproprylammonium tetrazolide (0.11 g, 0.65 mmol) in 3.0 mL of dichloromethane was added portion wise. The reaction mixture was further stirred for 16 hours at room temperature. The crude mixture was diluted with 50 mL of dichloromethane, washed sequentially with saturated NaHCO₃ solution (20 mL) and brine (20 mL), and dried over anhydrous Na₂SO₄. The solvent was evaporated in vacuo, and the crude mixture was purified by silica gel column chromatography using ethyl acetate/hexane solvent system (0-50% gradient on Combi Flash Rf Instrument) to give 0.12 g (20%) of product U2 (diastereomeric mixture) as white powder. ESI MS for C₄₆H₅₇FN₃O₉PS₂ Calculated 910.0, Observed 909 [M−H]⁺. ³¹P NMR (202 MHz, CDCl₃) δ151.3 (d, J 8.5 Hz), 151.2 (d, J 10.5 Hz).

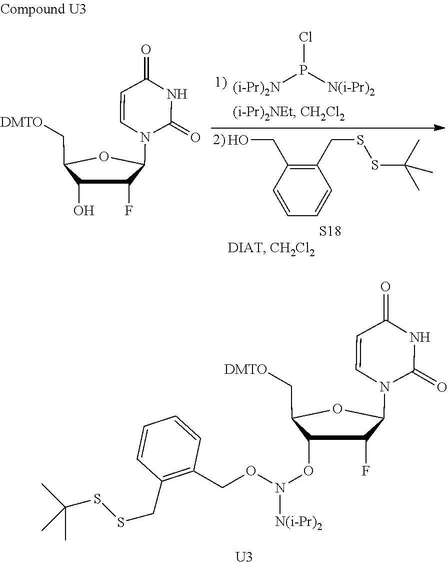

To a −78° C. cooled solution of 5′-O-(4,4′-dimethoxytrityl)-2′-F-uridine (0.73 g, 1.32 mmol) and N,N-diisopropylethylamine (0.25 mL, 1.45 mmol) in 15.0 mL of dry dichloromethane was added dropwise a solution of bis-(N,N-diisopropylamino) chlorophosphine (0.39 g, 1.45 mmol) in 5.0 mL of dichloromethane under argon atmosphere. The reaction mixture was allowed to warm to room temperature, while stirring was maintained (1 hour). A solution of S18 (0.32 g, 1.32 mmol) in 5.0 mL of dry dichloromethane was added dropwise, and the resulting mixture was stirred for 10 minutes, at which time a solution of ethylthiotetrazole in acetonitrile (0.25 M, 3.2 mL, 0.80 mmol) was added portion wise. The reaction mixture was further stirred for 3 hours at room temperature. The crude mixture was diluted with 100 mL of dichloromethane, washed sequentially with saturated NaHCO₃ solution (40 mL) and brine (40 mL), and dried over anhydrous Na₂SO₄. The solvent was evaporated in vacuo and the crude mixture was purified by silica gel column chromatography using ethyl acetate/hexane solvent system (0-50% gradient on Combi Flash Rf Instrument) to give 0.17 g (20%) of product U3 (diastereomeric mixture) as white powder. ESI MS for C₄₈H₅₉FN₃O₈PS₂ Calculated 920.0, Observed 943.0 [M+Na]⁺. ³¹P NMR (202 MHz, CDCl₃): δ156.3 (d, J 7.3 Hz), 155.6 (d, J 11.3 Hz).

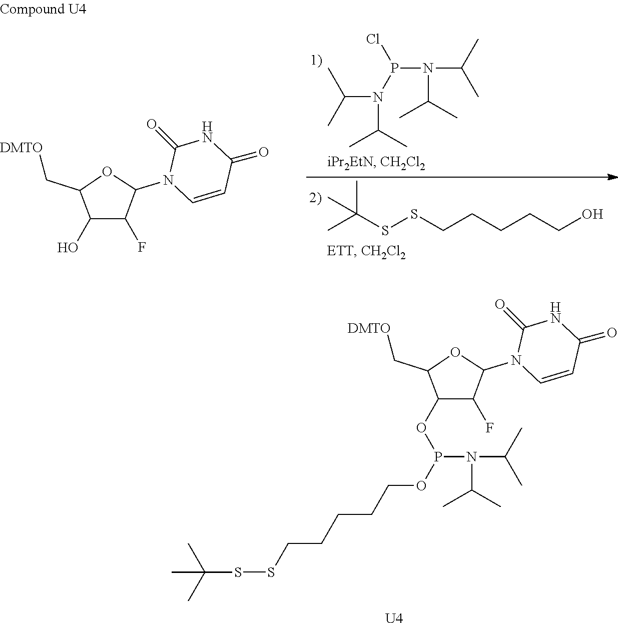

To a −78° C. cooled solution of 5′-O-(4,4′-dimethoxytrityl)-2′-F-uridine (1.77 g, 3.2 mmol) and N,N-diisopropylethylamine (0.62 mL, 3.54 mmol) in 20.0 mL of dry dichloromethane was added dropwise a solution of bis-(N,N-diisopropylamino)-chlorophosphine (0.94 g, 3.54 mmol) in 5.0 mL of dichloromethane under argon atmosphere. The reaction mixture was allowed to warm to room temperature, while stirring was maintained (1 hour). A solution of S20 (0.67 g, 3.22 mmol) in 5.0 mL of dry dichloromethane was added dropwise, and the resulting mixture was stirred for 10 minutes, at which time a solution of ethylthiotetrazole in acetonitrile (0.25M, 7.7 mL, 1.93 mmol) was added portionwise. The reaction mixture was further stirred for 3 hours at room temperature. The crude mixture was diluted with 100 mL of dichloromethane, washed sequentially with saturated NaHCO₃ solution (30 mL) and brine (30 mL), and dried over anhydrous Na₂SO₄. The solvent was evaporated in vacuo, and the crude mixture was purified by silica gel column chromatography using ethyl acetate/hexane solvent system (0-30% gradient on Combi Flash Rf Instrument) to give 1.48 g (52%) of product U4 (diastereomeric mixture) as a white powder. ESI MS for C₄₅H₆₁FN₃O₈PS₂ Calculated 886.08, Observed 884.8 [M−H]⁺. ³¹P NMR (202 MHz, CDCl₃) δ150.6 (d, J 6.8 Hz), 149.9 (d, J 9.1 Hz).

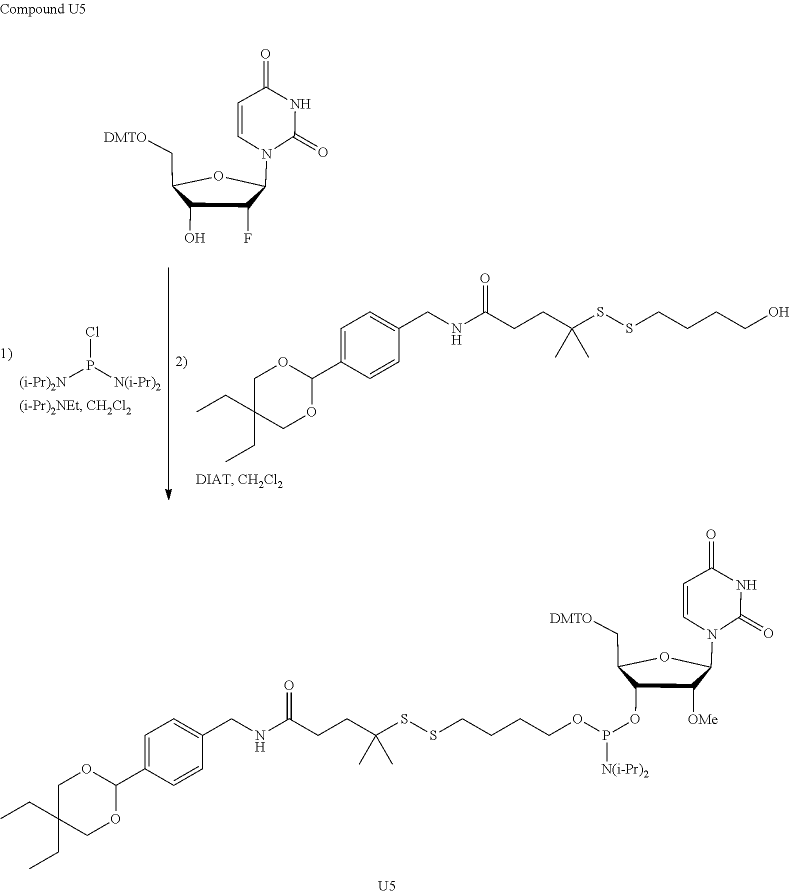

To a −78° C. cooled solution of 5′-O-(4,4′-dimethoxytrityl)-2′-F-uridine (0.66 g, 1.2 mmol) and N,N-diisopropylethylamine (0.23 mL, 1.32 mmol) in 10.0 mL of dry dichloromethane was added dropwise a solution of bis-(N,N-diisopropylamino)-chlorophosphine (0.35 g, 1.32 mmol) in 3.0 mL of dichloromethane under argon atmosphere. The reaction mixture was allowed to warm to room temperature, while stirring was maintained (1 hour). A solution of S23 (0.58 g, 1.2 mmol) in 3.0 mL of dry dichloromethane was added dropwise, and the resulting mixture was stirred for 10 minutes, at which time a solution of ethylthiotetrazole in acetonitrile (0.25M, 2.9 mL, 0.72 mmol) was added portionwise. The reaction mixture was further stirred for 3 hours at room temperature. The crude mixture was diluted with 50 mL of dichloromethane, washed sequentially by saturated NaHCO₃ solution (20 mL) and brine (20 mL), and dried over anhydrous Na₂SO₄. The solvent was evaporated in vacuo, and the crude mixture was purified by silica gel column chromatography using ethyl acetate/hexane solvent system (0-40% gradient on Combi Flash Rf Instrument) to give 0.35 g (27%) of product U5 (diastereomeric mixture) as white powder. ESI MS for C₆₁H₈₂FN₄O₁₁PS₂ Calculated 1161.42, Observed 1162 [M+H]⁺. ³¹P NMR (202 MHz, CDCl₃) δ154.87 (d, J 7.3 Hz), 154.53 (d, J 9.0 Hz).

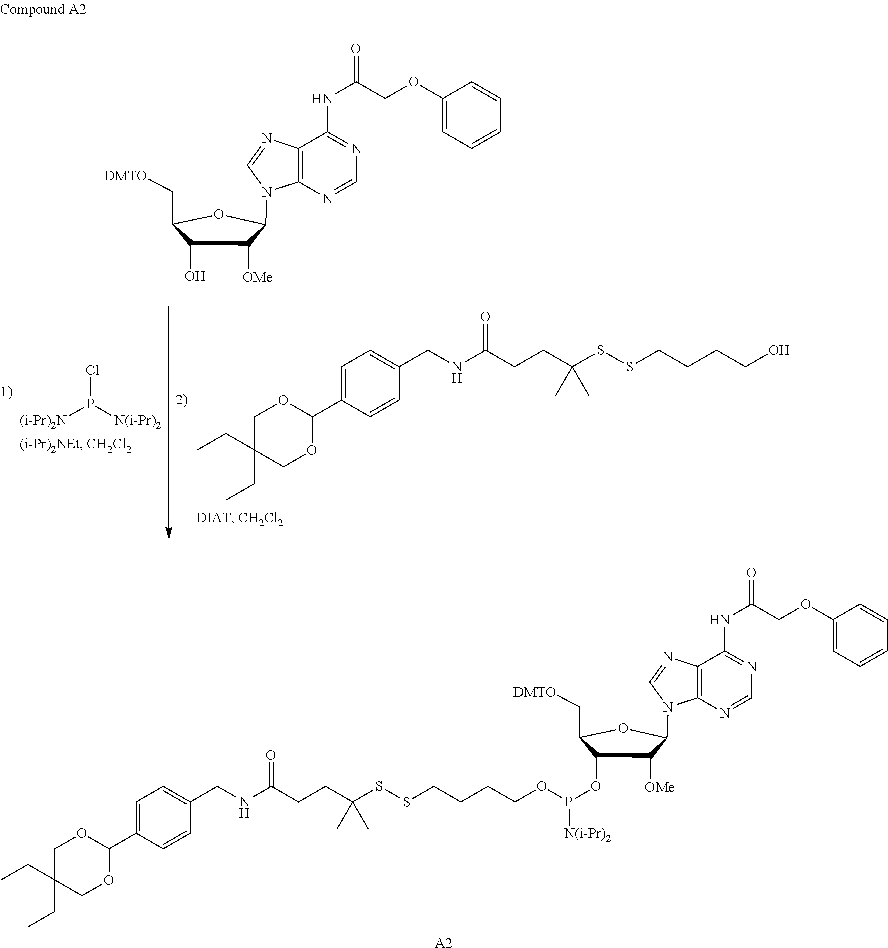

To a −78° C. cooled solution of 5′-O-(4,4′-dimethoxytrityl)-2′-O-methyl-adenosine (n-PAC) (1.48 g, 2.1 mmol) and N,N-diisopropylethylamine (0.4 mL, 2.28 mmol) in 15.0 mL of dry dichloromethane was added dropwise a solution of bis-(N,N-diisopropylamino) chlorophosphine (0.61 g, 2.28 mmol) in 5.0 mL of dichloromethane under argon atmosphere. The reaction mixture was allowed to warm to room temperature, while stirring was maintained (1 hour). A solution of S23 (1.0 g, 2.1 mmol) in 5.0 mL of dry dichloromethane was added dropwise, and the resulting mixture was stirred for 10 minutes, at which time a suspension of diisoproprylammonium tetrazolide (0.35 g, 2.1 mmol) in 5.0 mL of dichloromethane was added portionwise. The reaction mixture was further stirred for 16 hours at room temperature. The crude mixture was diluted with 75.0 mL of dichloromethane, washed sequentially by saturated NaHCO₃ solution (25 mL) and brine (25 mL), and dried over anhydrous Na₂SO₄. The solvent was evaporated in vacuo, and the crude mixture was purified by silica gel column chromatography using ethyl acetate/hexane solvent system (0-60% gradient on Combi Flash Rf Instrument) to give 1.01 g (37%) of product A2 (diastereomeric mixture) as a white powder. ESI MS for C₇₁H₉₂N₇O₁₂PS₂ Calculated 1330.63, Observed 1331.3 [M+H]⁺. ³¹P NMR (202 MHz, CDCl₃) δ154.93 & 154.29.

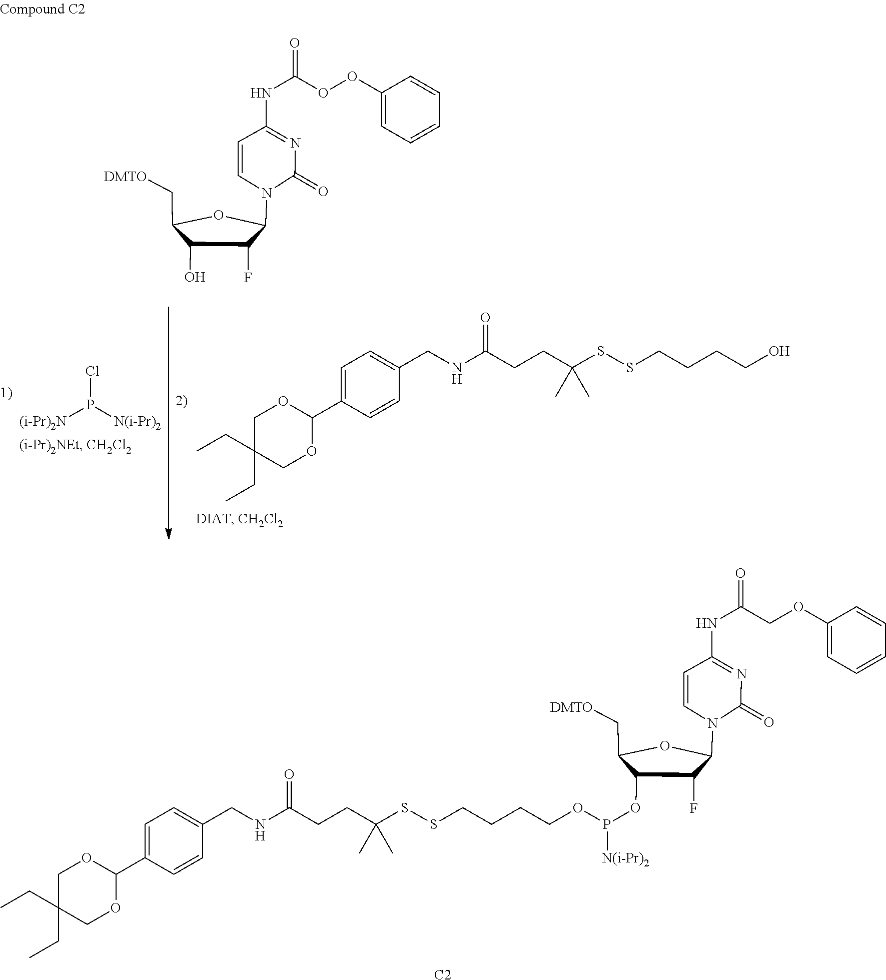

To a −78° C. cooled solution of 5′-O-(4,4-dimethoxytrityl)-2′-F-cytidine (n-PAC) (1.4 g, 2-1 mmol) and N,N-diisopropylethylamine (0.4 mL, 2.28 mmol) in 15.0 mL of dry dichloromethane was added dropwise a solution of bis-(N,N-diisopropylamino)-chlorophosphine (0.61 g, 2.28 mmol) in 5.0 mL of dichloromethane under Argon atmosphere. The reaction mixture was allowed to warm to room temperature while stirring was maintained (1 hour). A solution of S23 (1.0 g, 2.1 mmol) in 5.0 mL of dry dichloromethane was added dropwise, the resulting mixture was stirred for 10 minutes, at which time a suspension of diisoproprylammonium tetrazolide (0.35 g, 2.1 mmol) in 5.0 mL of dichloromethane was added portionwise. The reaction mixture was further stirred for 16 hours at room temperature. The crude mixture was diluted with 75 mL of dichloromethane, washed sequentially by saturated NaHCO₃ solution (25 mL) and brine (2 5 mL), and dried over anhydrous Na₂SO₄. The solvent was evaporated in vacuo, and the crude mixture was purified by silica gel column chromatography using ethyl acetate/hexane solvent system (0-50% gradient on Combi Flash Rf Instrument) to give 0.75 g (29%) of product C₂ (diastereomeric mixture) as a white powder. ESI MS for C₆₉H₈₉FN₅O₁₂PS₂ Calculated 1294.57, Observed 1295.2 [M+H]⁺. ³¹P NMR (202 MHz, CDCl₃) δ154.77 (d, J 5.6 Hz), 154.69 (d, J 7.7 Hz).

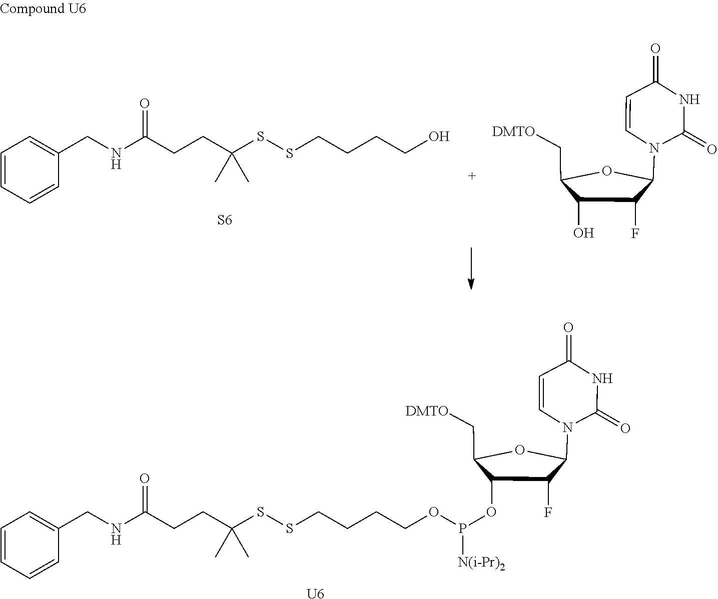

A solution of bis-(N,N-diisopropylamino)chlorophosphine (0.28 g, 1.05 mmol) in dry CH₂Cl₂ (1.0 mL) was added drop wise to a solution of 5′-(4,4′-dimethoxytrityl)-2′-F-uridine (0.55 g, 1.0 mmol) and N,N-diisopropylethylamine (0.18 mL, 1.05 mmol) in dry CH₂Cl₂ (5 mL) at −78° C. The reaction mixture was warmed to room temperature and stirred for 1.5 hours. A solution of S6 (0.34 g, 1.0 mmol) in 1.0 mL of dry CH₂Cl₂ was added, and the resulting mixture was stirred for 10 minutes. Then a solution of diisoproprylammonium tetrazolide (0.17 g, 1.0 mmol) in 8.0 mL of dry CH₂Cl₂ was added portionwise to the reaction mixture, and the resulting mixture was stirred overnight. The mixture was then diluted with CH₂Cl₂ (20 mL) and washed with saturated aqueous sodium bicarbonate (20 mL) and brine (20 mL). The mixture was dried over anhydrous sodium sulfate. Volatiles were evaporated in vacuo to afford a residue, which was subjected to flash silica gel column purification on an ISCO companion ((ethyl acetate with 5% methanol)/hexane=20%-55%) to give 0.50 g (49%) of compound U6 as a colorless foam. ESI MS for C₅₃H₆₈FN₄O₉PS₂ Calculated 1018.4, Observed 1018.1 (M⁺). ³¹P NMR (202 MHz, CDCl₃): δ150.15 (d, J 6.9 Hz), 149.65 (d, J 8.7 Hz).

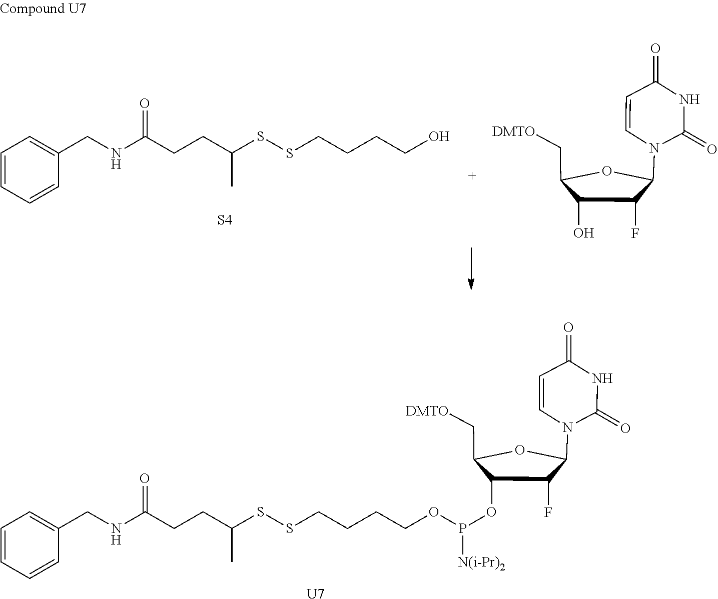

A solution of bis-(N,N-diisopropylamino)-chlorophosphine (0.28 g, 1.05 mmol) in dry CH₂Cl₂ (1.0 mL) was added drop wise to a solution of 5′-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S4 (0.33 g, 1.0 mmol) in 1.0 ml of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate, and the volatiles removed under vacuo to afford a residue, which was subjected to flash silica gel column purification on a ISCO companion ((ethyl acetate with 5% methanol)/hexane=20%-55%) to give 0.15 g (15% yield) of compound U7 as a colorless foam. ESI MS for C₅₂H₆₆FN₄O₉PS₂ Calculated 1004.4, Observed 1004.0 (M). ³¹P NMR (202 MHz, CDCl₃): δ 50.16 (d, J 7.9 Hz), 149.65 (d, J 10.7 Hz).

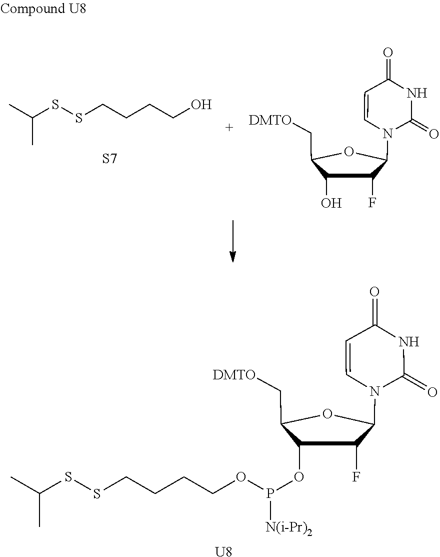

A solution of bis-(N,N-diisopropylamino)-chlorophosphine (0.28 g, 1.05 mmol) in dry CH₂Cl₂ (1.0 mL) was added dropwise to a solution of 5′-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S7 (0.18 g, 1.0 mmol) in 1.0 mL of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate and concentrated in vacuo to afford a residue, which was subjected to flash silica gel column purification on an ISCO companion ((ethyl acetate with 5% methanol)/hexane=10%-55%) to give 0.30 g (35%) of the title compound U8 as a colorless foam. ESI MS for C₄₃H₅₇FN₃O₈PS₂ Calculated 857.3, Observed 856.9 (M⁺). ³¹P NMR (202 MHz, CDCl₃): δ150.76 (d, J 7.7 Hz), 150.03 (d, J 9.3 Hz).

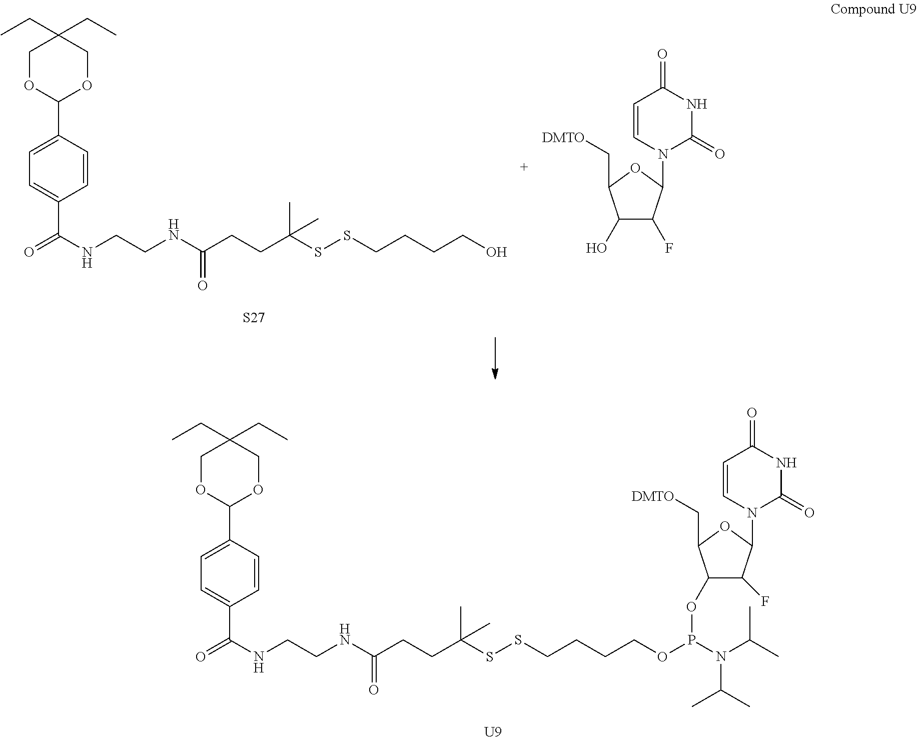

A solution of bis-(N, N-disiopropylamino)-chlorophosphine (0.28 g, 1.05 mmol) in dry CH₂Cl₂ (1.0 mL) was added dropwise to a solution of 5′-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture warmed to room temperature and stirred for 1.5 hours. A solution of S27 (0.54 g, 1.0 mmol) in 20.0 ml of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate, and the filtrate was evaporated in vacuo to afford a residue, which was subjected to flash silica gel column purification on an ISCO companion instrument (acetonitrile/dichloromethane=30%-90%) to give 0.68 g (56%) of the title compound U9 as a colorless foam. ESI MS for C₆₃H₈₅FN₅O₁₂PS₂ Calculated 1217.5, Observed 1217.2 (M⁺). ³¹P NMR (202 MHz, CDCl₃): δ150.18 (d, J 5.7 Hz), 148.40 (d, J 11.1 Hz).

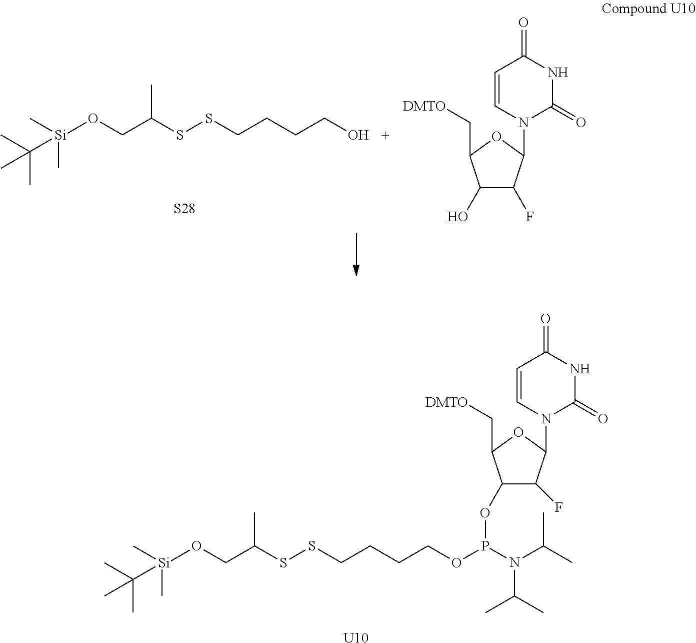

A solution of bis-(N,N-diisopropylamino)-chlorophosphine (0.16 g, 0.61 mmol) in dry CH₂Cl₂ (1.0 mL) was added dropwise to a solution of 5′-(4,4′-dimethoxytrityl)-2′-F-uridine (0.32 g, 0.58 mmol) and N,N-diisopropylethylamine (0.11 mL, 0.61 mmol) in dry CH₂Cl₂ (5 mL) at −78° C. The reaction mixture was warmed to room temperature and stirred for 1.5 hours. A solution of S28 (0.18 g, 0.58 mmol) in 1.0 mL of dry CH₂Cl₂ was added, and the resulting mixture was stirred for 10 minutes. Then a solution of diisopropylammonium tetrazolide (0.10 g, 0.61 mmol) in 8.0 mL of dry CH₂Cl₂ was added portionwise to the reaction mixture, and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate, and the volatiles were evaporated in vacuo to afford a residue, which was subjected to flash silica gel column purification on an ISCO companion instrument ((ethyl acetate with 5% methanol)/hexane=10%-55%) to give 0.15 g (26%) of the title compound U10 as a colorless foam. ESI MS for C₄₉H₇₁FN₃O₉PS₂Si Calculated 987.4, Observed 987.0 (M⁺). ³¹P NMR (202 MHz, CDCl₃): δ150.88 (s), 150.08 (d, J 9.3 Hz).

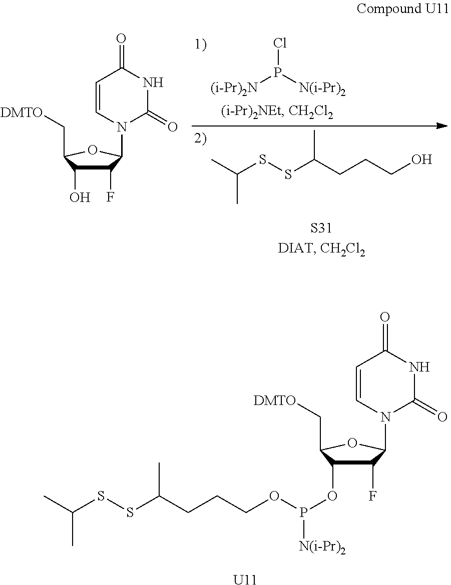

A solution of bis-(N,N-diisopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-diisopropylethylamine (0.18 ml, 1.05 mmol) in dry CH₂Cl₂ (5.0 mL) at −78° C. The reaction mixture was warmed to room temperature and stirred for 1.5 hours. A solution of S31 (0.18 g, 1.0 mmol) in 1.0 mL of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portion wise to the reaction mixture, and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate and concentrated in vacuo to afford a residue, which was subjected to flash silica gel column purification on a ISCO companion ((ethyl acetate with 5% methanol)/hexane=10%-55%) to give 0.38 g (44%) the title compound U11 as a colorless foam. ESI MS for C₄₄H₅₉FN₃O₈PS₂ Calculated 871.3, Observed 870.8 (M⁺). ³¹P NMR (202 MHz, CDCl₃): δ150.84 (d, J 7.6 Hz), 150.73 (d, J 7.6 Hz) 150.06 (d, J 9.1 Hz), 150.02 (d, J 9.1 Hz).

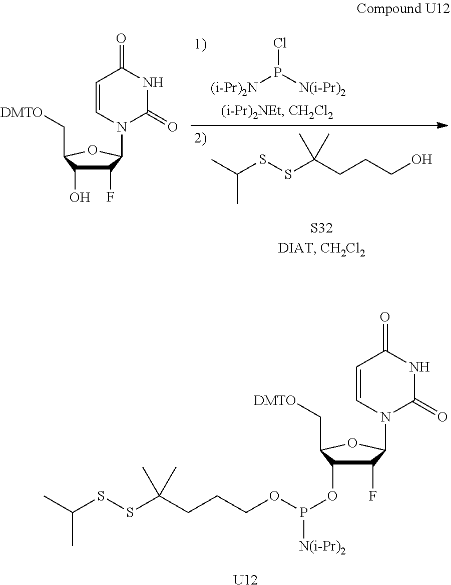

A solution of bis-(N, N-disiopropylamino)-chlorophosphine (0.28 g, 1.05 mmol) in dry CH₂Cl₂ (1.0 ml) was added dropwise to a solution of S32 (0.18 g, 1.0 mmol) and N,N-diisopropylethylamine (0.18 mL, 1.05 mmol) in dry CH₂Cl₂ (5.0 mL) at −78° C. The reaction mixture warmed to room temperature and stirred for 1.5 hours. A solution of 5′-(4,4′-dimethoxytrityl)-2′-F-uridine (0.55 g, 1.0 mmol) in 1.0 mL of dry CH₂Cl₂ was added, and the resulting mixture was stirred for 10 minutes. Then a solution of 2-ethylthiotetrazole (2.4 mL, 0.25M in acetonitrile, 0.6 mmol) was added portionwise to the reaction mixture, and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate, and the filtrate was evaporated in vacuo to afford a residue, which was subjected to flash silica gel column purification on a ISCO companion (ethyl acetate/hexane=10% -55%) to give 0.47 g (53%) of the title compound U12 as a colorless foam. ESI MS for C₄₅H₆₁FN₃O₈PS₂ Calculated 885.4, Observed 884.7 (M-1). ³¹P NMR (202 MHz, CDCl₃): δ150.88 (d, J 7.7 Hz), 150.03 (d, J 9.5 Hz).

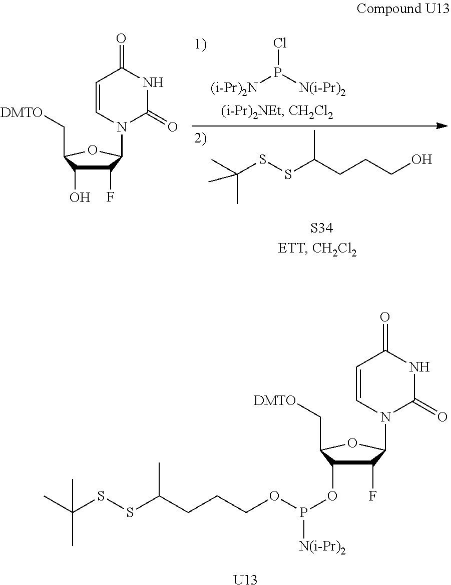

A solution of bis-(N,N-diisopropylamino)-chlorophosphine (0.26 g, 0.97 mmol) in dry CH₂Cl₂ (1.0 ml) was added dropwise to a solution of S34 (0.19 g, 0.92 mmol) and N, N-diisopropylethylamine (0.17 mL, 0.97 mmol) in dry CH₂Cl₂ (5.0 mL) at −78° C. The reaction mixture was warmed to room temperature and stirred for 1.5 hours. A solution of 5′-(4,4′-dimethoxytrityl)-2′-F-uridine (0.50 g, 0.92 mmol) in 1.0 mL of dry CH₂Cl₂ was added, and the resulting mixture was stirred for 10 minutes. Then a solution of 2-ethylthiotetrazole (ETT) (2.6 mL, 0.25M in acetonitrile, 0.65 mmol) was added portionwise to the reaction mixture, and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate, and the filtrate was evaporated in vacuo to afford a residue, which was subjected to flash silica gel column purification on a ISCO companion (ethyl acetate/hexane=10% -55%) to give 0.29 g (36%) of the title compound U13 as a colorless foam. ESI MS for C₄₅H₆₁FN₃O₈PS₂ Calculated 885.4, Observed 885.2 (M⁺). ³¹P NMR (202 MHz, CDCl₃): δ150.91 (d, J 7.7 Hz), 150.76 (d, J 7.7 Hz), 150.07 (d, J 9.1 Hz), 150.02 (d, J 9.5 Hz).

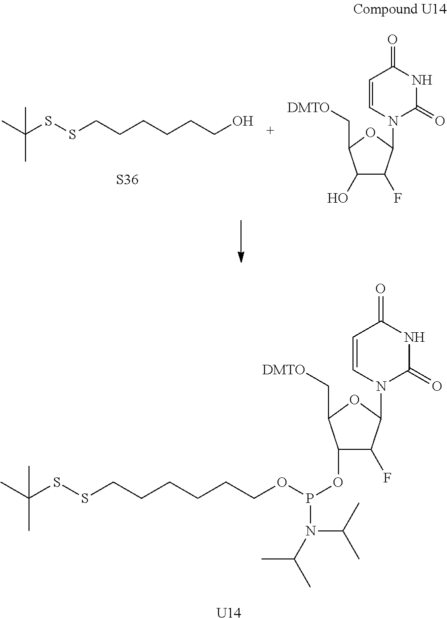

A solution of bis-(N,N-diisopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S36 (0.22 g, 1.0 mmol) in 1.0 mL of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The reaction mixture was diluted with CH₂Cl₂ (20 mL) and washed with saturated aqueous sodium bicarbonate (20 mL) and brine (20 mL). The mixture was dried over anhydrous sodium sulfate, and the filtrate was concentrated in vacuo to afford a residue, which was subjected to flash silica gel column purification on an ISCO companion ((ethyl acetate with 5% methanol)/hexane=10%-55%) to give 0.37 g (41%) of the title compound U14 as a colorless foam. ESI MS for C₄₆H₆₃FN₃OPS₂ Calculated 899.4, Observed 900.7 (M+1). ³¹P NMR (202 MHz, CDCl₃): δ155.32 (d, J 7.7 Hz), 154.72 (d, J 9.3 Hz).

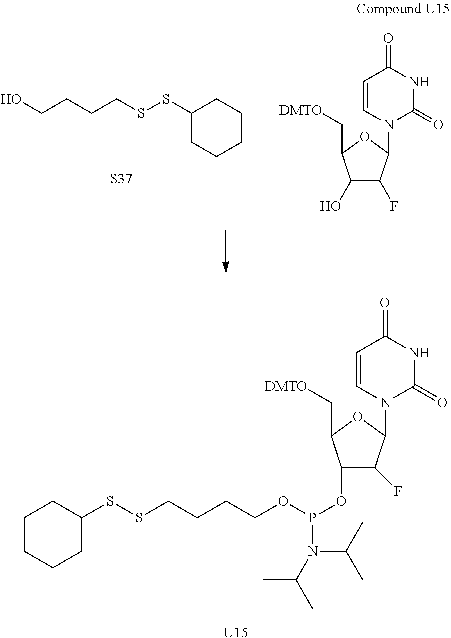

A solution of bis-(N,N-diisopropylamino)-chlorophosphine (0.28 g, 1.55 mmol) in dry CH₂Cl₂ (1.0 mL) was added dropwise to a solution of 5′-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S37 (0.22 g, 1.0 mmol) in 1.0 mL of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The reaction mixture was diluted with CH₂Cl₂ (20 mL) and washed with saturated aqueous sodium hydrogen carbonate (20 mL) and brine (20 mL). The organic layer was dried over anhydrous sodium sulfate, and the filtrate was concentrated in vacuo to afford a residue, which was subjected to flash silica gel column purification on an ISCO companion ((ethyl acetate with 5% methanol)/hexane=10%-55%) to give 0.34 g (38%) of the title compound U15 as a colorless foam. ESI MS for C₄₆H₆₁FN₃O₈PS₂ Calculated 897.4, Observed 896.7 (M-1). ³¹P NMR (202 MHz, CDCl₃): δ150.73 (d, J 7.7 Hz), 150.01 (d, J 9.5 Hz).

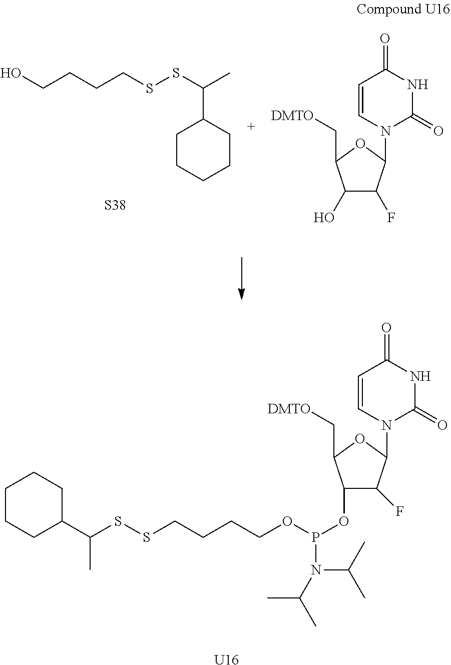

A solution of bis-(N, N-disiopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-Diisopropylethylamine (0.18 mL, 1.05 mmol) in dry CH₂Cl₂ (5.0 mL) at −78° C. The reaction mixture was warmed to room temperature and stirred for 1.5 hours. A solution of S38 (0.25 g, 1.0 mmol) in 1.0 ml of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium hydrogen carbonate (20 mL) and brine (20 mL). The organic layer was dried over anhydrous sodium sulfate and the filtrate was concentrated in vacuo to afford a residue, which was subjected to flash silica gel column purification on a ISCO companion (ethyl acetate with 5% methanol/hexane=10%-55%) to give 0.38 g (41%) of the title compound U16 as a colorless foam. ESI MS for C₄₈H₆₅FN₃O₈PS₂Calculated 925.4, Observed 926.5 (M+1). ³¹P NMR (202 MHz, CDCl₃): δ150.78 (d, J 6.9 Hz), 150.02 (d, J 9.5 Hz).

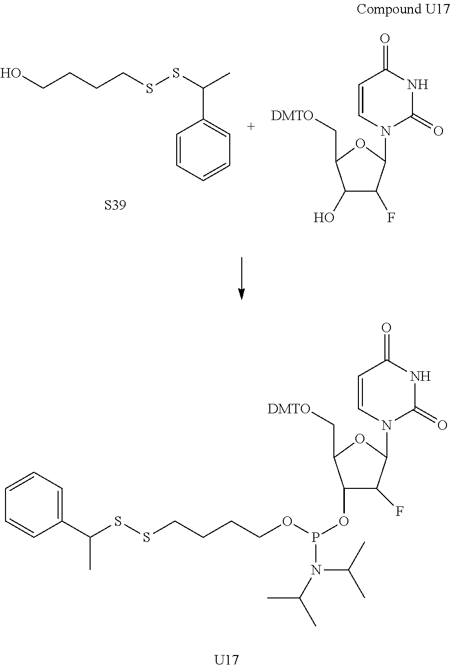

A solution of bis-(N, N-disiopropylamino)-chlorophosphine (0.28 g, 1.05 mmol) in dry CH₂Cl₂ (1.0 mL) was added dropwise to a solution of 5′-(4,4′-dimethoxytrityl)-2′-F-uridine (0.55 g, 1.0 mmol) and N,N-Diisopropylethylamine (0.18 mL, 1.05 mmol) in dry CH₂Cl₂ (5.0 mL) at −78° C. The reaction mixture was warmed to room temperature and stirred for 1.5 hours. A solution of S39 (0.24 g, 1.0 mmol) in 1.0 mL of dry CH₂Cl₂ was added and stirred for 10 minutes. Then a solution of diisopropylammonium tetrazolide (0.18 g, 1.05 mmol) in 8.0 mL of dry CH₂Cl₂ was added portion wise to the reaction mixture and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate and the filtrate was concentrated in vacuo to afford a residue, which was subjected to flash silica gel column purification on a ISCO companion (ethyl acetate with 5% methanol/hexane=10%-55%) to give 0.24 g (26%) of the title compound U17 as a colorless foam. ESI MS for C₄₈H₅₉FN₃OPS₂ Calculated 919.3, Observed 920.7 (M+1). ³¹P NMR (202 MHz, CDCl₃): δ155.41 (d, J 7.1 Hz), 154.73 (d, J 8.9 Hz).

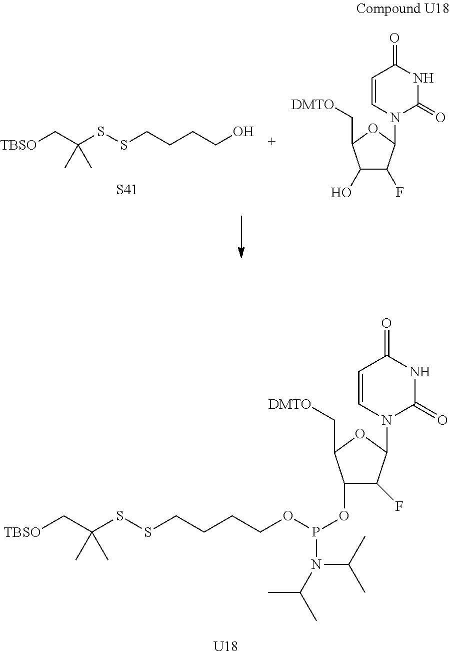

A solution of bis-(N,N-diisopropylamino)-chlorophosphine (0.28 g, 1.05 mmol) in dry CH₂Cl₂ (1.0 mL) was added dropwise to a solution of 5′-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S41 (0.32 g, 1.0 mmol) in 1.0 mL of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The reaction mixture was diluted with CH₂Cl₂ (20 mL) and washed with saturated aqueous sodium bicarbonate (20 mL) and brine (20 mL). The organic layer was dried over anhydrous sodium sulfate, and the filtrate was concentrated in vacuo to afford a residue, which was subjected to flash silica gel column purification on a ISCO companion ((ethyl acetate with 5% methanol)/hexane=10%-55%) to give 0.25 g (25%) of the title compound U18 as a colorless foam. ESI MS for C₅₀H₇₃FN₃O₉PS₂Si Calculated 1001.4, Observed 1003.1 (M+2). ³¹P NMR (202 MHz, CDCl₃): δ155.67 (d, J 7.7 Hz), 154.81 (d, J 9.7 Hz).

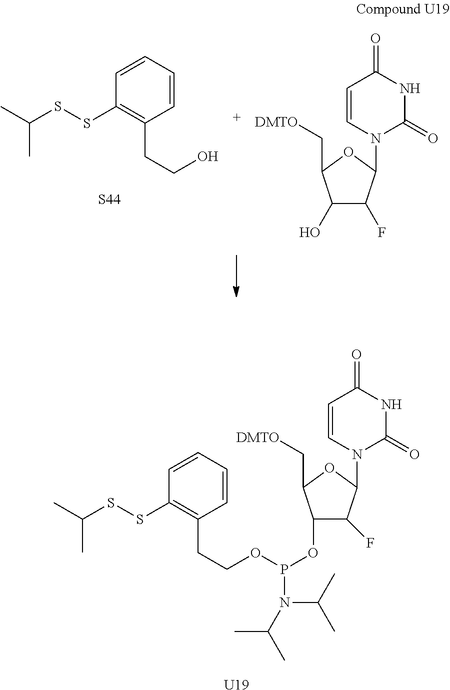

A solution of bis-(N,N-diisopropylamino)-chlorophosphine (0.28 g, 1.05 mmol) in dry CH₂Cl₂ (1.0 mL) was added dropwise to a solution of 5′-O-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S44 (0.23 g, 1.0 mmol) in 1.0 mL of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The mixture was diluted with CH₂Cl₂ (20 mL) and washed with saturated aqueous sodium bicarbonate (20 mL) and brine (20 mL). The mixture was dried over anhydrous sodium sulfate, and the filtrate was concentrated in vacuo to afford a residue, which was subjected to flash silica gel column purification on a ISCO companion ((ethyl acetate with 5% methanol)/hexane=10%-55%) to give 0.24 g (27%) of the title compound U19 as a colorless foam. ESI MS for C₄₇H₅₇FN₃O₈PS₂ Calculated 905.3, Observed 907.0 (M+2). ³¹P NMR (202 MHz, CDCl₃): δ154.74 (d, J 8.9 Hz), 154.53 (d, J 7.7 Hz).

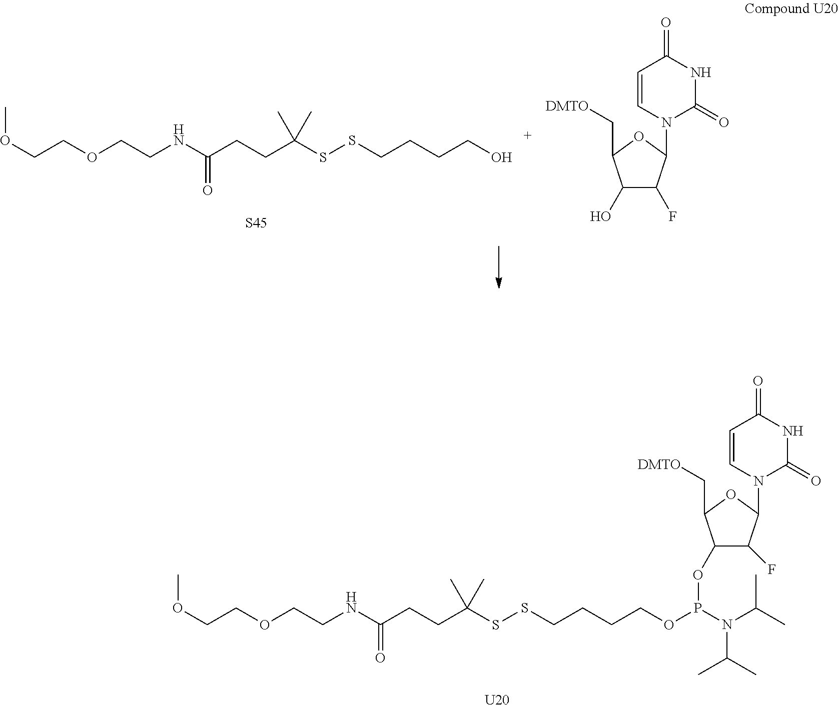

A solution of bis-(N,N-diisopropylamino)-chlorophosphine (0.57 g, 2.14 mmol) in dry CH₂Cl₂ (2.0 mL) was added dropwise to a solution of 5′-(4,4′-dimethoxytrityl)-2′-F-uridine (1.11 g, 2.0 mmol) and N,N-diisopropylethylamine (0.37 mL, 2.14 mmol) in dry CH₂Cl₂ (10.0 mL) at −78° C. The reaction mixture was warmed to room temperature and stirred for 1.5 hours. A solution of S45 (0.72 g, 2.0 mmol) in 5.0 mL of dry CH₂Cl₂ was added, and the resulting mixture was stirred for 10 minutes. Then a solution of diisopropylammonium tetrazolide (0.37 g, 2.14 mmol) in 8.0 mL of dry CH₂Cl₂ was added portionwise to the reaction mixture, and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate, and the filtrate was concentrated in vacuo to afford a residue, which was subjected to flash silica gel column purification on a ISCO companion (EtOAc/Hexane, containing 2.5% MeOH) to give 0.45 g (23%) of the title compound U20 as a colorless oil. ³¹P NMR (202 MHz, CDCl₃): δ150.13 (d, J 6.5 Hz), 149.13 (d, J 9.1 Hz)

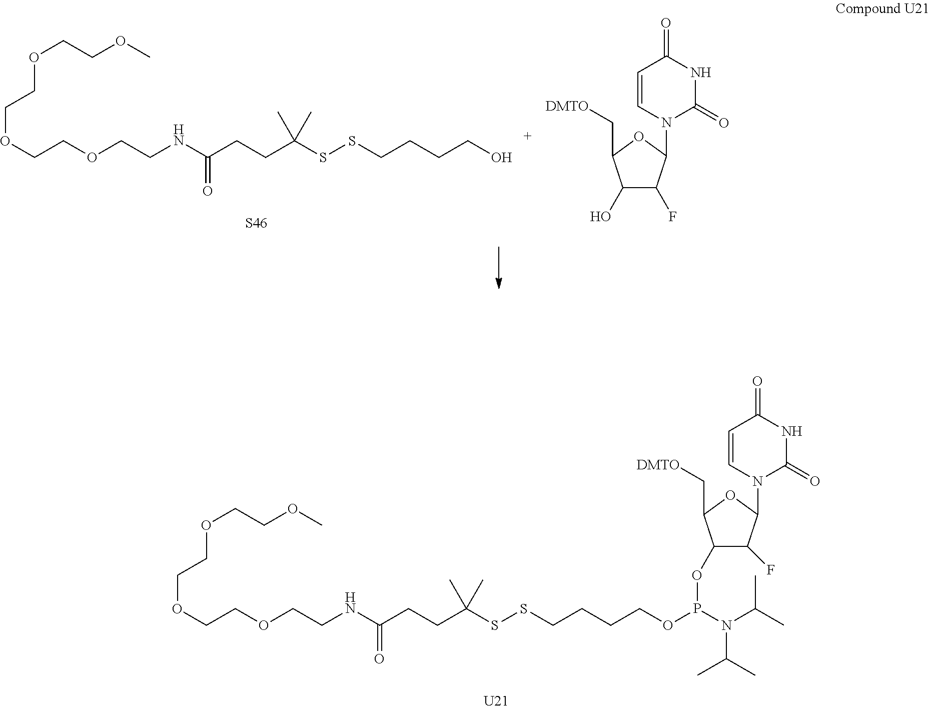

A solution of bis-(N,N-diisopropylamino)-chlorophosphine (0.28 g, 1.05 mmol) in dry CH₂Cl₂ (1.0 mL) was added dropwise to a solution of 5′-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S46 (0.44 g, 1.0 mmol) in 1.0 ml of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate, and the filtrate was concentrated in vacuo to afford a residue, which was subjected to flash silica gel column purification on an ISCO companion (methanol/dichloromethane=1%-8%) to give 0.30 g (27%) of the title compound U21 as a colorless oil. ESI MS for C₅₅H₈₀FN₄O₁₃PS₂ Calculated 1118.5, Observed 1118.3 (M⁺). ³¹P NMR (202 MHz, CDCl₃): δ150.15 (d, J 6.5 Hz), 149.23 (d, J 9.1 Hz).

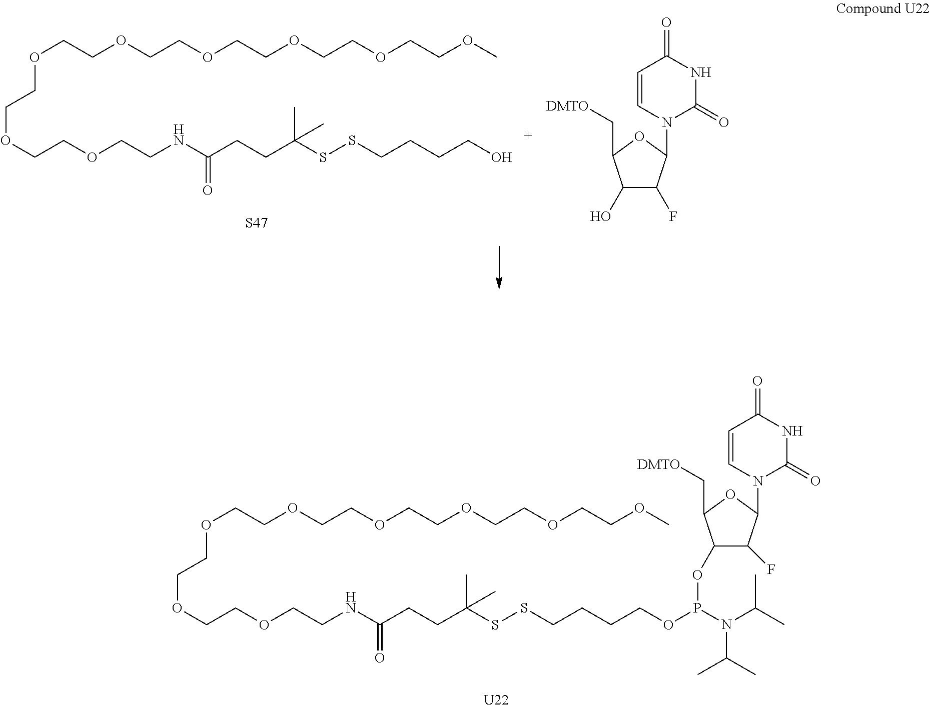

A solution of bis-(N,N-diisopropylamino)-chlorophosphine (0.38 g, 1.41 mmol) in dry CH₂Cl₂ (1.0 ml) was added dropwise to a solution of 5′-O-(4,4′-dimethoxytrityl)-2′-F-uridine (0.74 g, 1.34 mmol) and N,N-diisopropylethylamine (0.25 mL, 1.41 mmol) in dry CH₂Cl₂ (5.0 mL) at −78° C. The reaction mixture was warmed to room temperature and stirred for 1.5 hours. A solution of S47 (0.75 g, 1.22 mmol) in 1.0 mL of dry CH₂Cl₂ was added, and the resulting mixture was stirred for 10 minutes. Then a solution of diisopropylammonium tetrazolide (0.24 g, 1.41 mmol) in 10 mL of dry CH₂Cl₂ was added portionwise to the reaction mixture, and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate, and the filtrate was concentrated in vacuo to afford a residue, which was subjected to flash silica gel column purification on a ISCO companion (methanol/dichloromethane=1%-8%) to give 0.56 g (32%) the title compound U22 as a colorless oil. ESI MS for C₆₃H₉₆FN₄O₁₇PS₂ Calculated 1294.6, Observed 1294.4 (M⁺). ³¹P NMR (202 MHz, CDCl₃): δ150.15 (d, J 7.1 Hz), 149.21 (d, J 9.5 Hz).

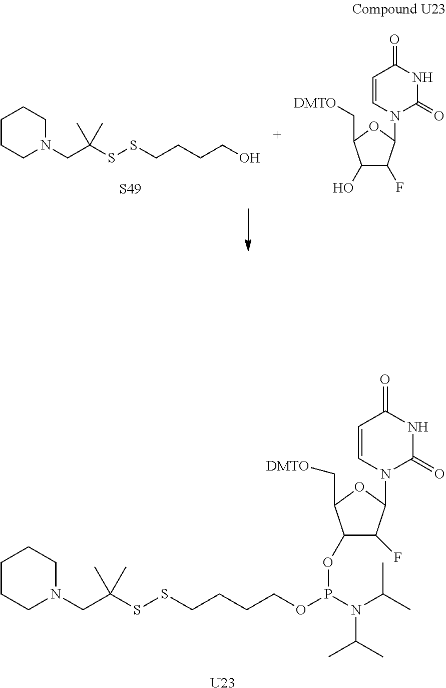

A solution of bis-(N,N-disiopropylamino)-chlorophosphine (0.28 g, 1.05 mmol) in dry CH₂Cl₂ (1.0 mL) was added dropwise to a solution of 5′-(4,4′-dimethoxytrityl)-2′-F-uridine (0.55 g, 1.0 mmol) and N,N-diisopropylethylamine (0.18 mL, 1.05 mmol) in dry CH₂Cl₂ (5.0 mL) at −78° C. The reaction mixture was warmed to room temperature and stirred for 1.5 hours. A solution of S49 (0.32 g, 1.0 mmol) in 1.0 ml of dry CH₂Cl₂ was added, and the resulting mixture was stirred for 10 minutes. Then a solution of diisopropylammonium tetrazolide (0.18 g, 1.05 mmol) in 8.0 mL of dry CH₂Cl₂ was added portionwise to the reaction mixture, and the resulting mixture was stirred overnight. The mixture was diluted with CH₂Cl₂ (20 mL) and washed with saturated aqueous sodium bicarbonate (20 mL) and brine (20 mL). The organic layer was dried over anhydrous sodium sulfate, and the filtrate was concentrated in vacuo to afford a residue, which was subjected to flash silica gel column purification on an ISCO companion (ethyl acetate/hexane=5%-80%) to give 0.34 g (36%) of the title compound U23 as a colorless foam. ESI MS for C₄₉H₆₈FN₄O₈PS₂Calculated 954.4, Observed 955.9 (M+1). ³¹P NMR (202 MHz, CDCl₃): δ155.54 (d, J 7.0 Hz), 154.80 (d, J 8.3 Hz).

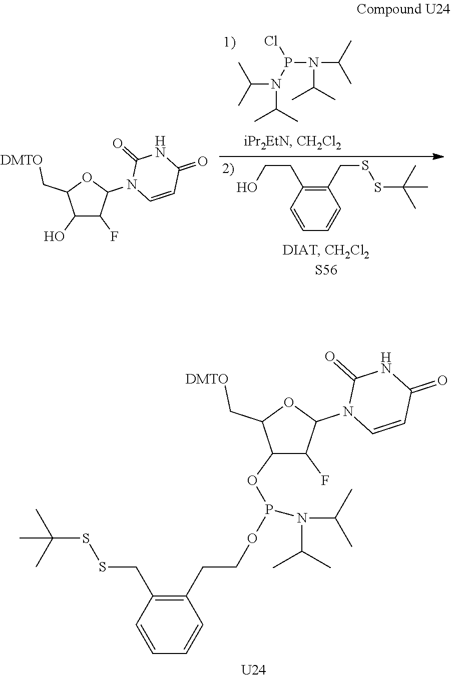

Procedure 1/Protocol 1: To a cooled solution (−78° C.) of 5′-O-(4,4′-dimethoxytrityl)-2′-F-uridine (1.93 g, 3.52 mmol) and N,N-diisopropylethylamine (680 μL, 3.87 mmol) in 20.0 mL of dry dichloromethane was added dropwise a solution of bis-(N,N-diisopropylamino)-chlorophosphine (1.03 g, 3.87 mmol) in 10.0 mL of dichloromethane under argon atmosphere. The reaction mixture was allowed to warm to room temperature, while stirring was maintained (1 hour). To this mixture, a solution of S56 (0.90 g, 3.52 mmol) in 5.0 mL of dry dichloromethane was added dropwise, and the resulting mixture was stirred for 10 minutes, at which time a suspension of diisoproprylammonium tetrazolide (0.66 g, 3.87 mmol) in 5.0 mL of dichloromethane was added portionwise. The reaction mixture was further stirred for 16 hours at room temperature. The reaction mixture was diluted with 200 mL of dichloromethane and washed sequentially by saturated NaHCO₃ solution (40.0 mL) and brine (40.0 mL), then dried over anhydrous Na₂SO₄. The solvent was evaporated in vacuo, and the crude mixture was purified by silica gel column chromatography using ethyl acetate/hexane solvent system (0-30% gradient on Combi Flash Rf Instrument) to give product U24 as a white powder (1.1 g, 33% yield). ESI MS for C₄₉H₆₁FN₃O₈PS₂ calculated 934.1, observed 934.9 [M+H]⁺. ³¹P NMR (202 MHz, CDCl₃) δ155.3 (d, J 8.7 Hz), 154.7 (d, J 8.9 Hz)

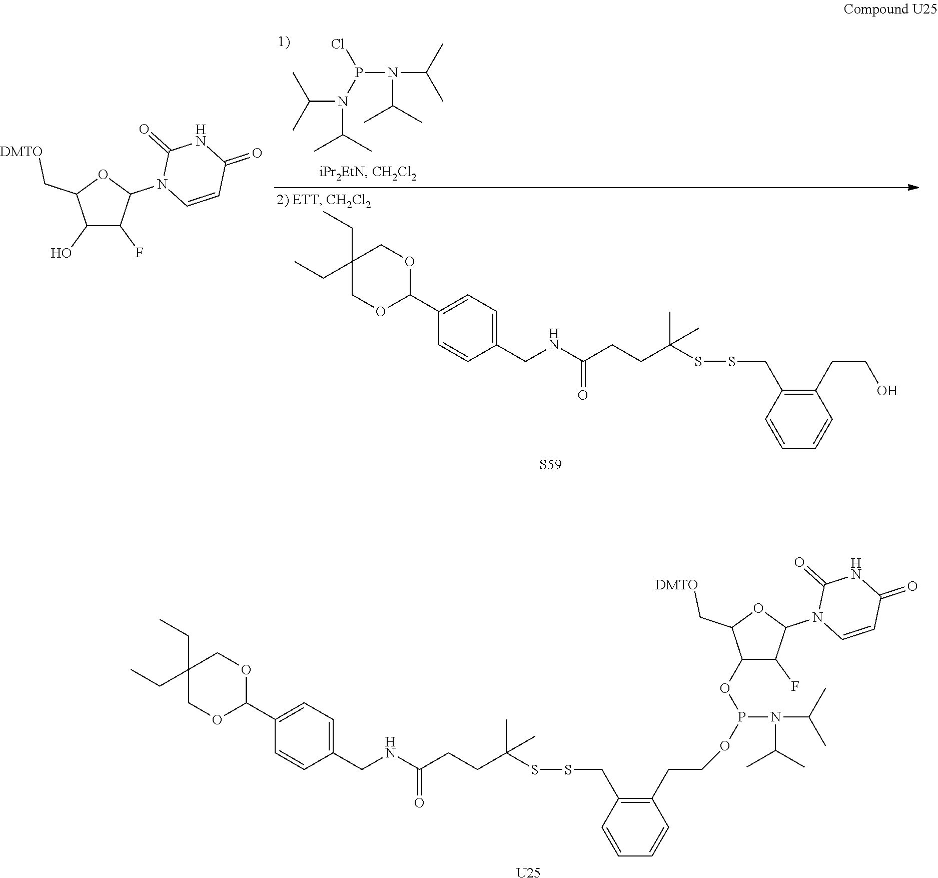

Procedure 2/Protocol 2: To a cooled solution (−78° C.) of 5′-O-(4,4′-dimethoxytrityl)-2′-F-uridine (0.60 g, 1.1 mmol) and N,N-diisopropylethylamine (211 μL, 1.21 mmol) in 10.0 mL of dry dichloromethane was added dropwise a solution of bis-(N,N-diisopropylamino)-chlorophosphine (0.32 g, 1.21 mmol) in 5.0 mL of dichloromethane under argon atmosphere. The reaction mixture was allowed to warm to room temperature, while stirring was maintained (1 hour). A solution of S59 (0.60 g, 1.1 mmol) in 5.0 mL of dry dichloromethane was added dropwise, and the resulting mixture was stirred for 10 minutes, at which time a solution of ethylthiotetrazole (ETT) in acetonitrile (0.25M, 2.6 mL, 0.66 mmol) was added portionwise. The reaction mixture was further stirred for 3 hours at room temperature. The crude mixture was diluted with 50.0 mL of dichloromethane, washed sequentially by saturated NaHCO₃ solution (25.0 mL) and brine (25.0 mL), and dried over anhydrous Na₂SO₄. The solvent was evaporated in vacuo, and the crude mixture was purified by silica gel column chromatography using ethyl acetate/hexane solvent system (0-50% gradient on Combi Flash Rf Instrument) to give product U25 as white powder (0.77 g, 58% yield). ESI MS for C₆₆H₈₄FN₄O₁₁PS₂ calculated 1223.5, observed [M+H]⁺1224.2. ³¹P NMR (202 MHz, CDCl₃) δ154.8 (d, J 7.0 Hz), 154.6 (d, J 9.5 Hz)

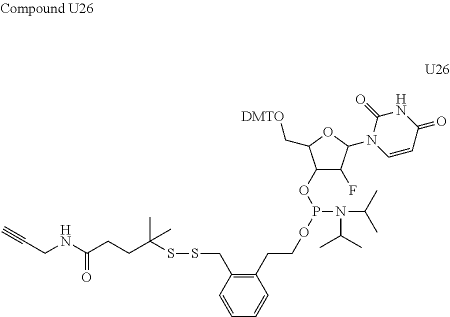

Compound U26 was prepared from alkyl disulfide (prepared from compounds S68 and S55 according to the procedure described for compound S59) and 5′-(4,4′-dimethoxytrityl)-2′-F-uridine employing procedure 2.

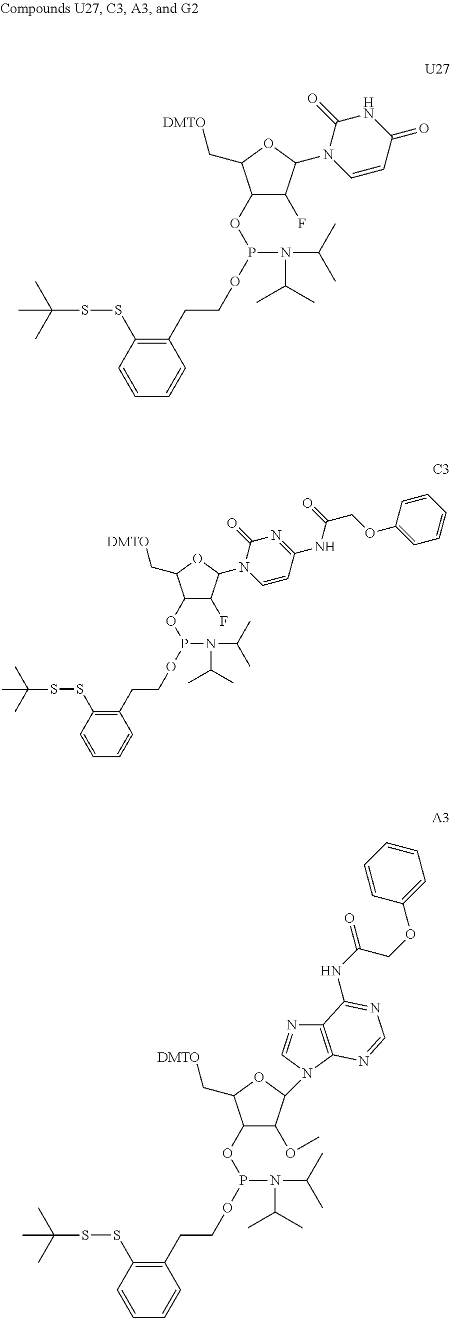

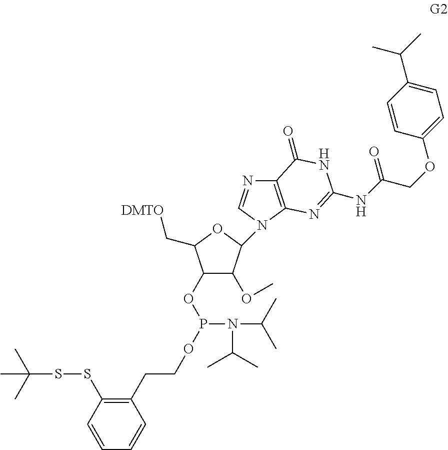

Compound U27 was prepared from compound S61 according to Protocol 1 (see compound U24) in 41% yield. ESI MS for C₄₈H₅₉FN₃O₈PS₂ calculated 920.1, observed 920.9 [M+H]⁺. ³¹P NMR (202 MHz, CDCl₃) δ154.7 (d, J 8.9 Hz), 154.5 (d, J 7.7 Hz)

Compound C3 was prepared according to Protocol 1 (see compound U24) in 59% yield. ESI MS for C₅₆H₆₆FN₄OPS₂ calculated 1053.2, observed 1051.5 [M−H]⁺. ³¹P NMR (202 MHz, CDCl₃) δ154.6 (d, J 5.45 Hz), 154.4 (d, J 8.3 Hz)

Compound A3 was prepared according to Protocol 1 (see compound U24) in 39% yield. ESI MS for C₅₈H₆₉FN₆O₉PS₂ calculated 1089.3, observed 1090.2[M+H]⁺. ³¹P NMR (202 MHz, CDCl₃) δ154.8 (s), 154.6(s)

Compound G2 can be prepared from, e.g., compound S61, according to methods described herein.

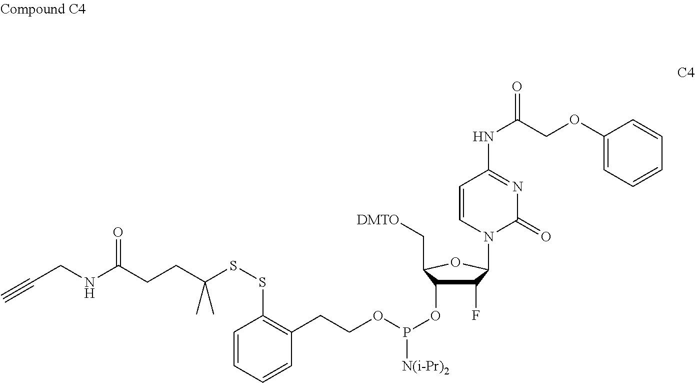

Compound C4 was prepared according to Procedure 2 (see compound U25) in 22% yield. ESI MS for C₆₁H₇₁FN₅O₁₀PS₂ calculated 1148.3, observed 1147.0 [M−H]⁺. ³¹P NMR (202 MHz, CDCl₃) δ 154.7 (d, J 5.05 Hz), 154.1 (d, J 10.7 Hz)

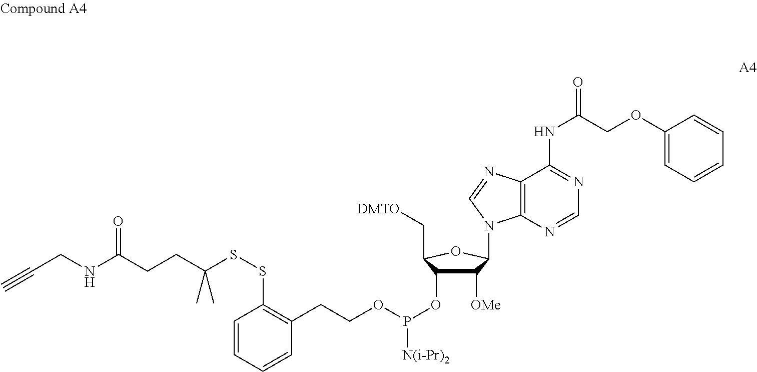

Compound A4 was prepared according to Procedure 2 (see compound U25) in 18% yield. ESI MS for C₆₃H₇₄N₇O₁₀PS₂ calculated 1184.4, observed 1183.2 [M−H]⁺. ³¹P NMR (202 MHz, CDCl₃) 5154.7 (s), 154.1 (s)

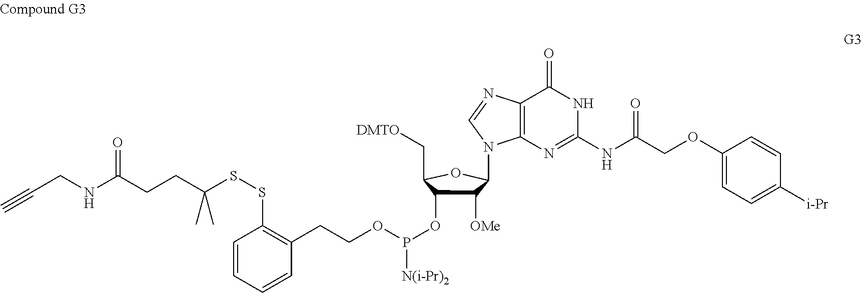

Compound G3 was prepared according to Procedure 2 (see compound U25).

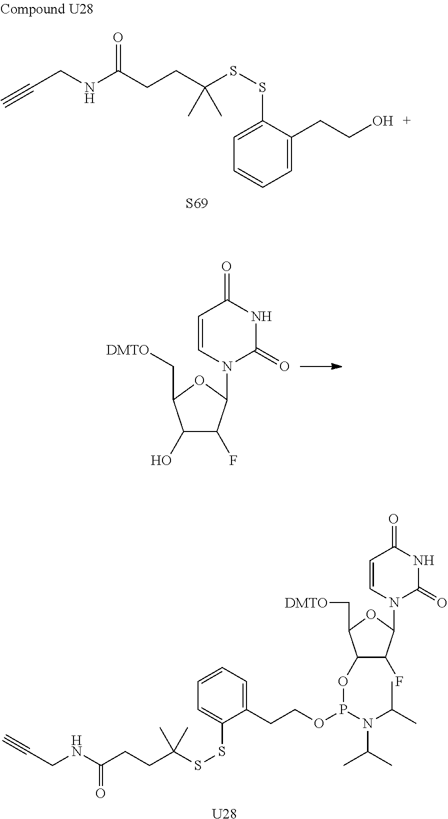

Compound U28 was prepared according to Procedure 1 (see compound U24). ESI MS for C₅₃H₆₄FN₄O₉PS₂Calculated 1015.2, Observed 1016.2 (M+1). ³¹P NMR (202 MHz, CDCl₃): δ154.79 (d, J 7.5 Hz), 154.38 (d, J 10.5 Hz)

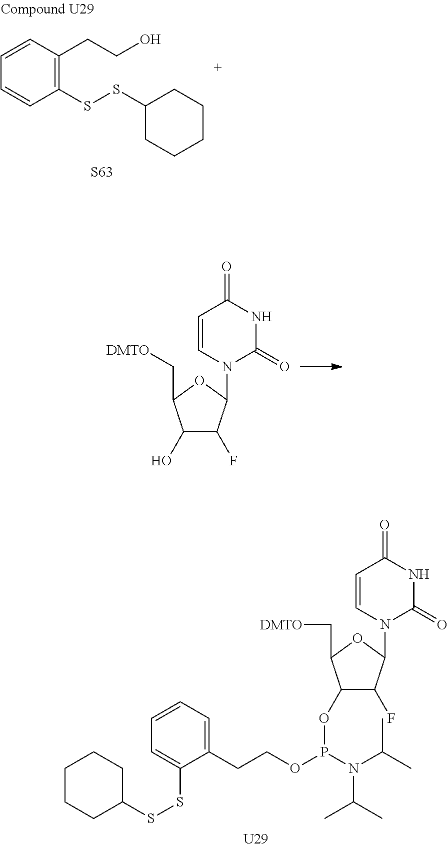

Compound U29 was prepared according to Procedure 1 (see compound U24). ESI MS for C₅₀H₆₁FN₃O₈PS₂ Calculated 946.1, Observed 947.6 (M+1). ³¹P NMR (202 MHz, CDCl₃): δ154.74 (d, J 7.7 Hz), 154.50 (d, J 7.7 Hz)

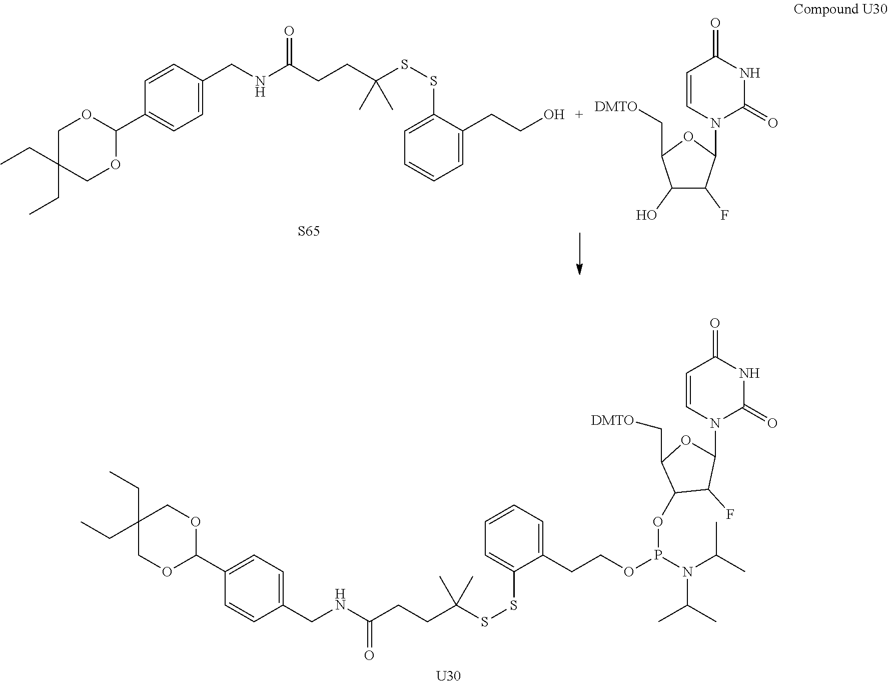

Compound U30 was prepared according to procedure 2 (see compound U25). ESI MS for C₆₅H₈₂FN₄O₁₁PS₂Calculated 1209.5, Observed 1210.6 (M+1). ³¹P NMR (202 MHz, CDCl₃): δ154.74 (d, J 6.7 Hz), 154.34 (d, J 10.3 Hz)

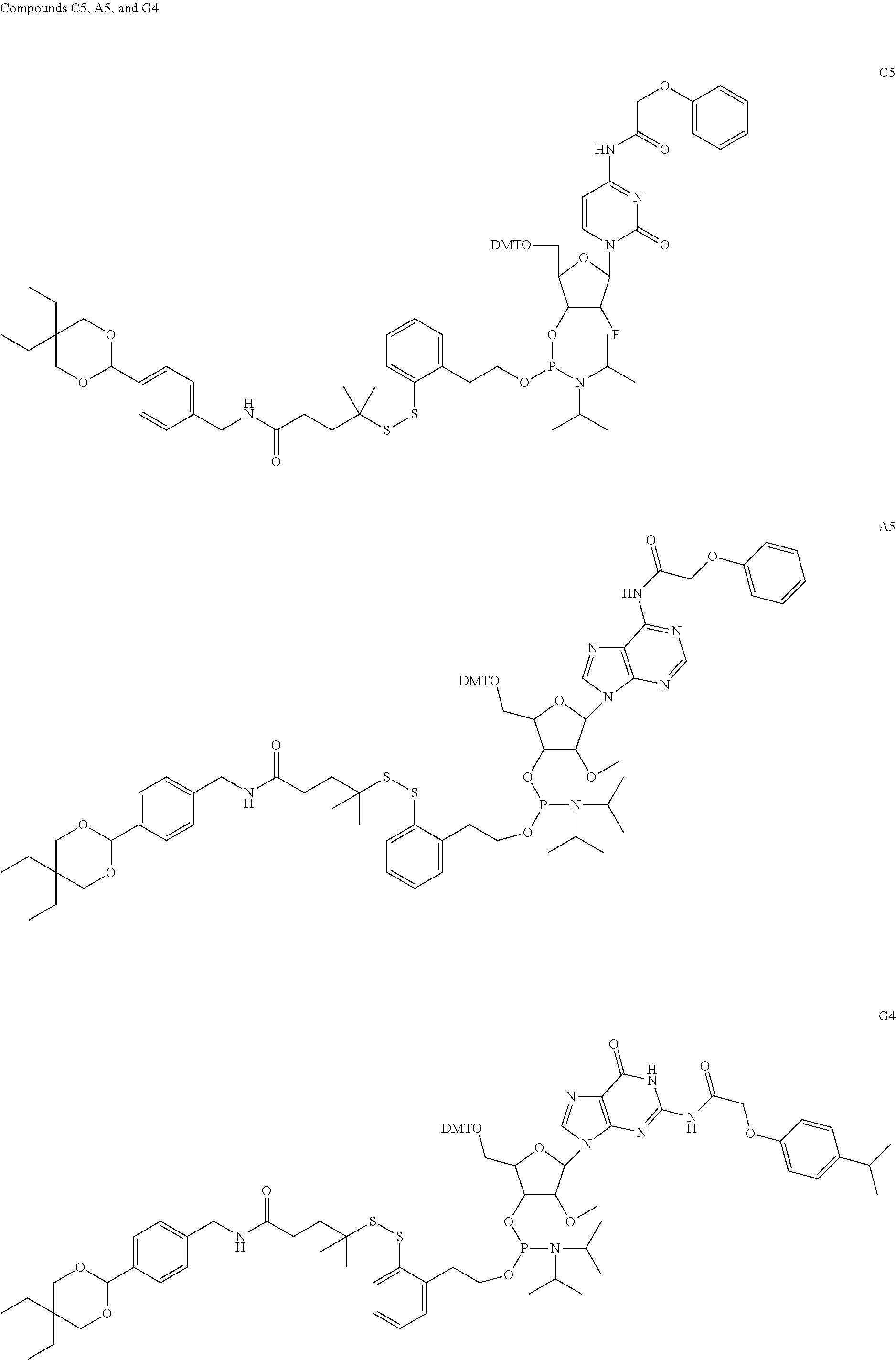

Compounds C₅, A5, and G4 are prepared according to procedure 2 (see compound U25).

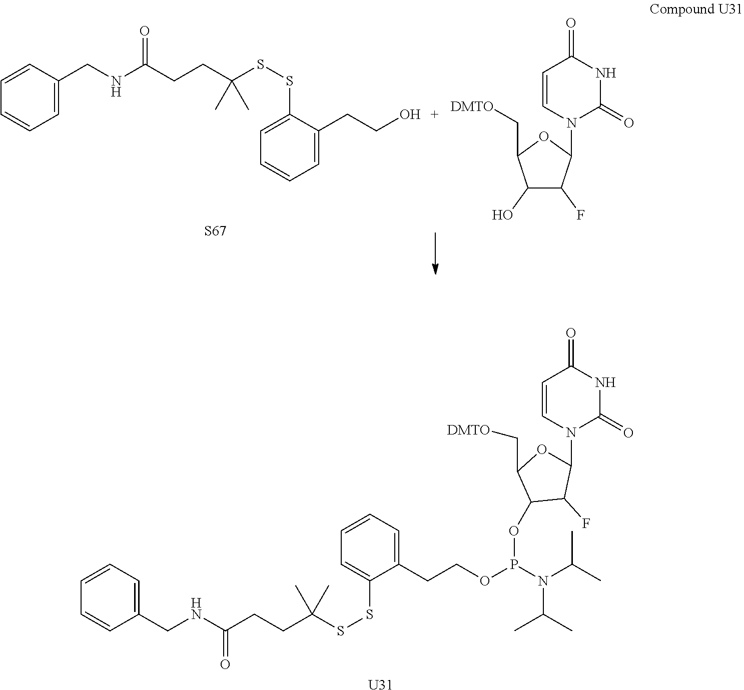

Compound U31 was prepared according to procedure 1 (see compound U24). ESI MS for C₅₇H₆₈FN₄O₉PS₂Calculated 1067.3, Observed 1065.6 (M-1). ³¹P NMR (202 MHz, CDCl₃): δ154.76 (d, J 7.4 Hz), 154.49 (d, J 10.1 Hz)

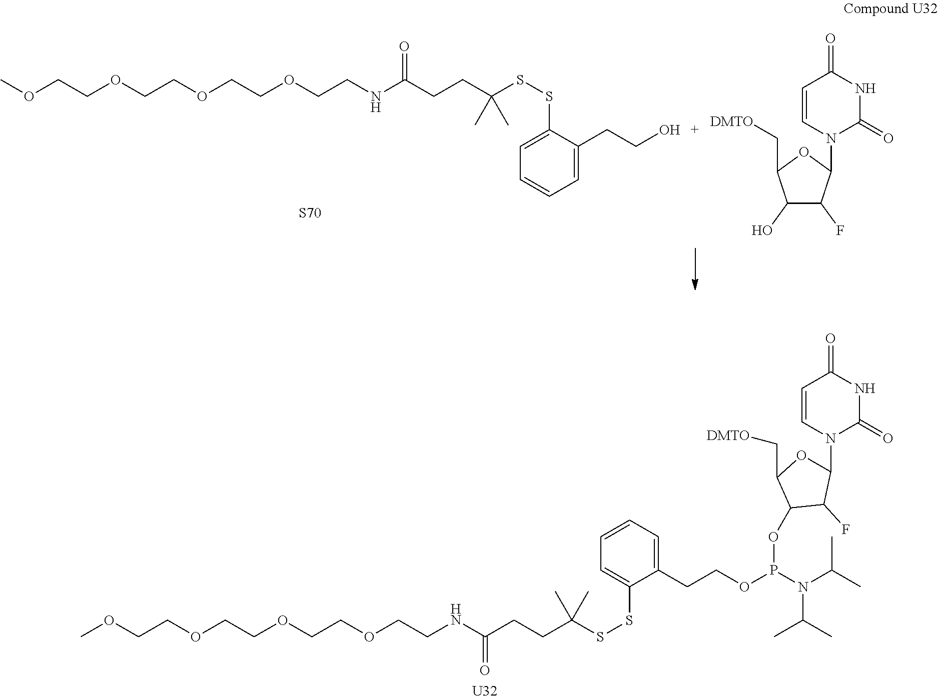

Compound U32 was prepared according to procedure 1 (see compound U24). ESI MS for C₅₉H₈₀FN₄O₁₃PS₂Calculated 1167.4, Observed 1166.5 (M-1). ³¹P NMR (202 MHz, CDCl₃): δ154.71 (d, J 7.3 Hz), 154.00 (d, J 10.9 Hz)

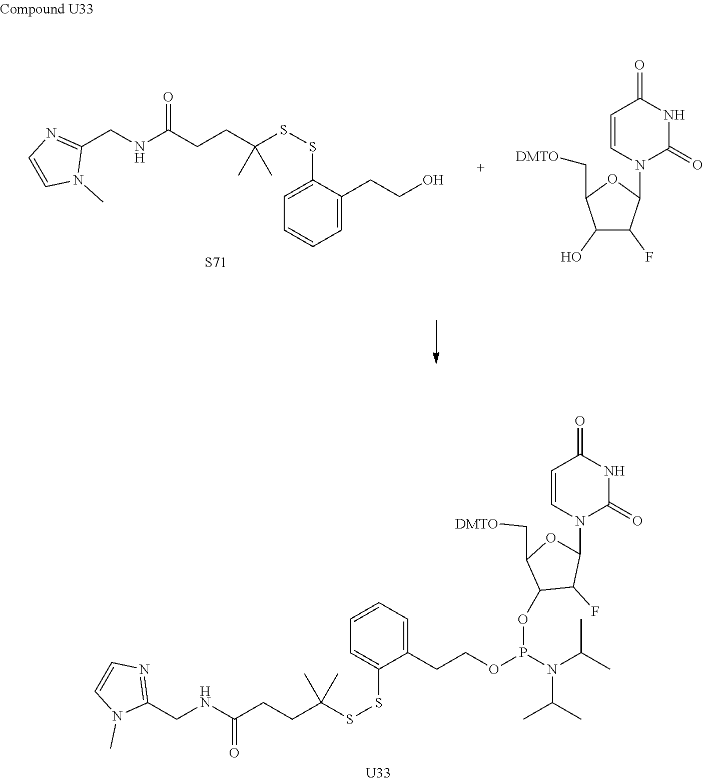

Compound U33 was prepared according to procedure 1 (see compound U24). ESI MS for C₅₅H₆₈FN₆O₉PS₂ Calculated 1071.3, Observed 1072.1 (M+1). ³¹P NMR (202 MHz, CDCl₃): δ 155.09 (s), 152.98 (d, J 14.9 Hz)

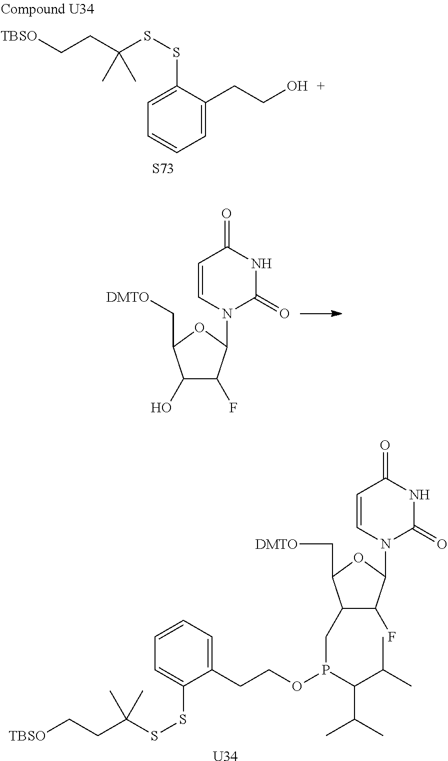

Compound U34 was prepared according to procedure 1 (see compound U24). ESI MS for C₅₅H₇₅FN₃O₉PS₂Si Calculated 1064.4, Observed 1065.1 (M+1). ³¹P NMR (202 MHz, CDCl₃): δ154.81 (d, J 8.9 Hz), 154.56 (d, J 7.9 Hz)

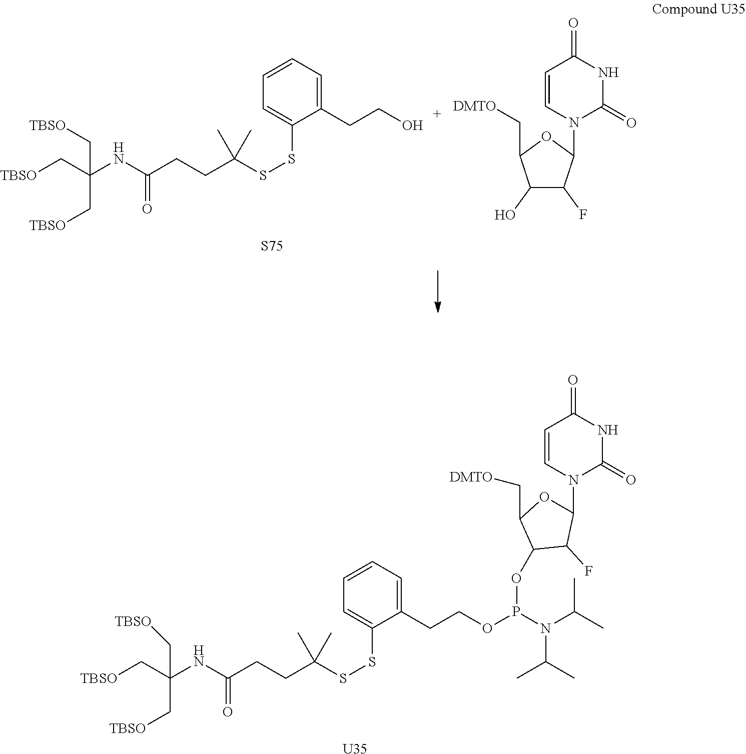

Compound U35 was prepared according to procedure 1 (see compound U24). ³¹P NMR (202 MHz, CDCl₃): δ154.62 (d, J 7.3 Hz), 154.50 (d, J 9.2 Hz)

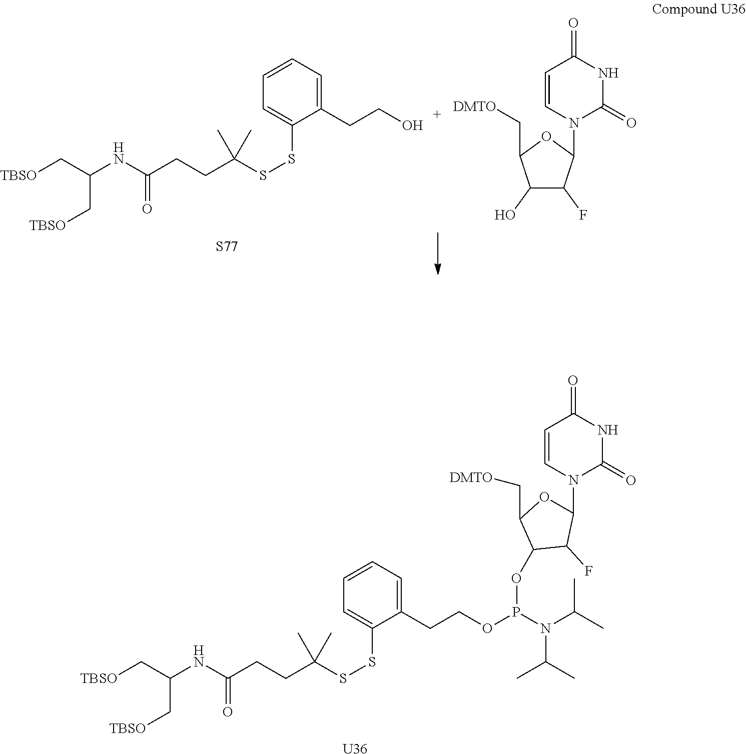

Compound U36 was prepared according to procedure 1 (see compound U24). ESI MS for C₆₅H₉₆FN₄O₁₁PS₂Si₂ Calculated 1279.8, Observed 1278.5 (M-1). ³¹P NMR (202 MHz, CDCl₃): δ154.72 (d, J 7.1 Hz), 154.60 (d, J 9.1 Hz)

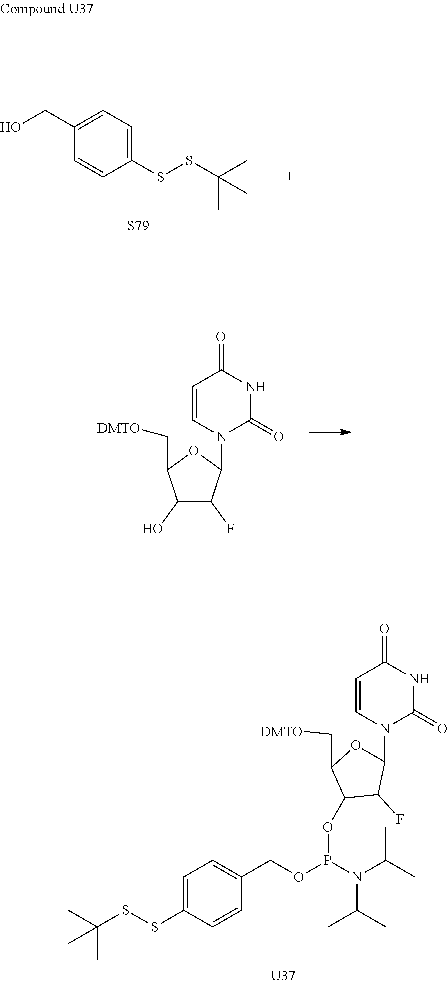

Compound U37 was prepared according to procedure 1 (see compound U24). ESI MS for C₄₇H₅₇FN₃O₈PS₂ Calculated 906.1, Observed 906.7 (M+1). ³¹P NMR (202 MHz, CDCl₃): δ156.35 (d, J 8.5 Hz), 155.98 (d, J 8.7 Hz)

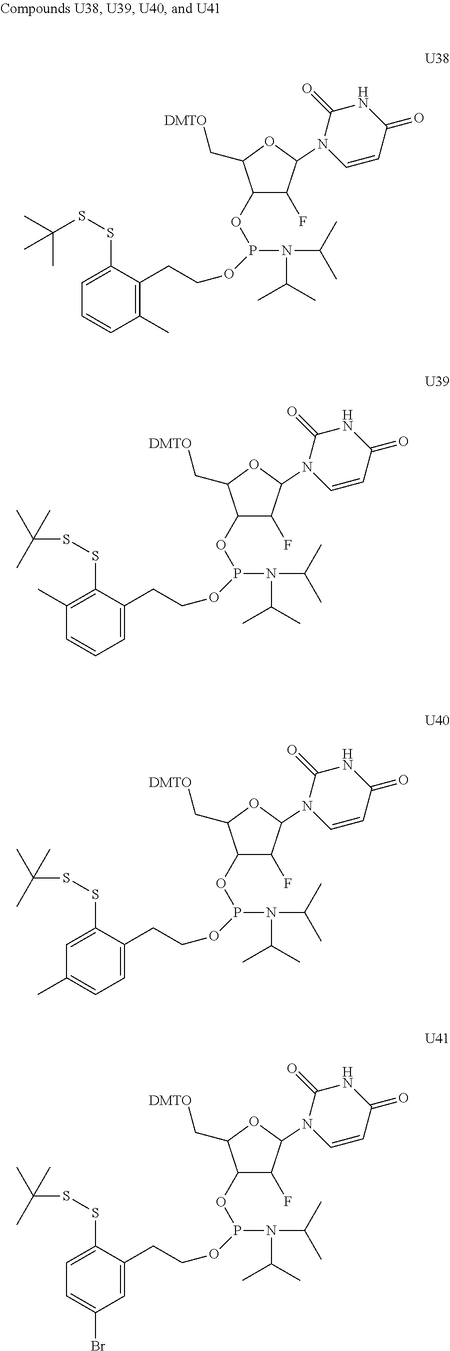

Compounds U38, U39, U40 and U41 were prepared according to procedure 1 (see compound U24).

U38: ESI MS for C₄₉H₆₁FN₃O₈PS₂Calculated 934.1, Observed 933.1 (M-1). ³¹P NMR (202 MHz, CDCl₃): δ154.74 (d, J 7.7 Hz), 154.70 (d, J 7.9 Hz)

U39: ESI MS for C₄₉H₆₁FN₃O₈PS₂ Calculated 934.1, Observed 844.8 (M-t-BuS). ³¹P NMR (202 MHz, CDCl₃): δ154.81 (d, J 8.7 Hz), 154.58 (d, J 8.3 Hz)

U40: ESI MS for C₄₉H₆₁FN₃O₈PS₂Calculated 934.1, Observed 933.5 (M-1). ³¹P NMR (202 MHz, CDCl₃): δ154.64 (d, J 8.3 Hz), 154.53 (d, J 7.9 Hz)

U41: ESI MS for C₄₈H₅₈BrFN₃O₈PS₂ Calculated 999.0, Observed 999.9 (M+1). ³¹P NMR (202 MHz, CDCl₃): δ155.47 (d, J 7.7 Hz), 154.74 (d, J 8.7 Hz)

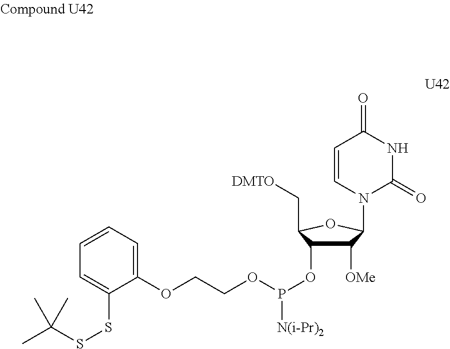

Compound U42 was prepared from compound S83 according to procedure 1 (see compound U24).

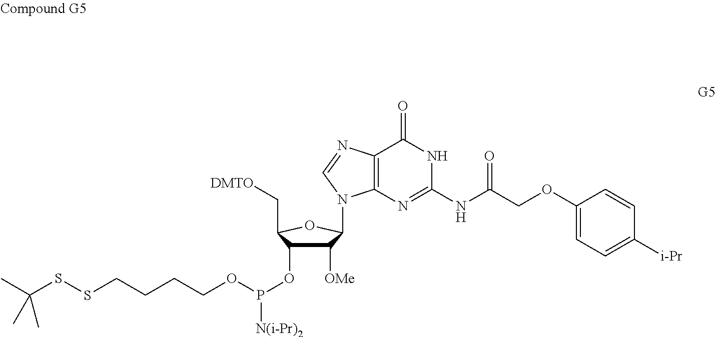

Compound G5 was prepared as described herein. ESI MS for C₅₇H₇₅N₆O₁₀PS₂ calculated 1099.34, observed [M−H]⁺ 1098.2. ³¹P NMR (202 MHz, CDCl₃) δ 150.48 (s), 149.87 (s)

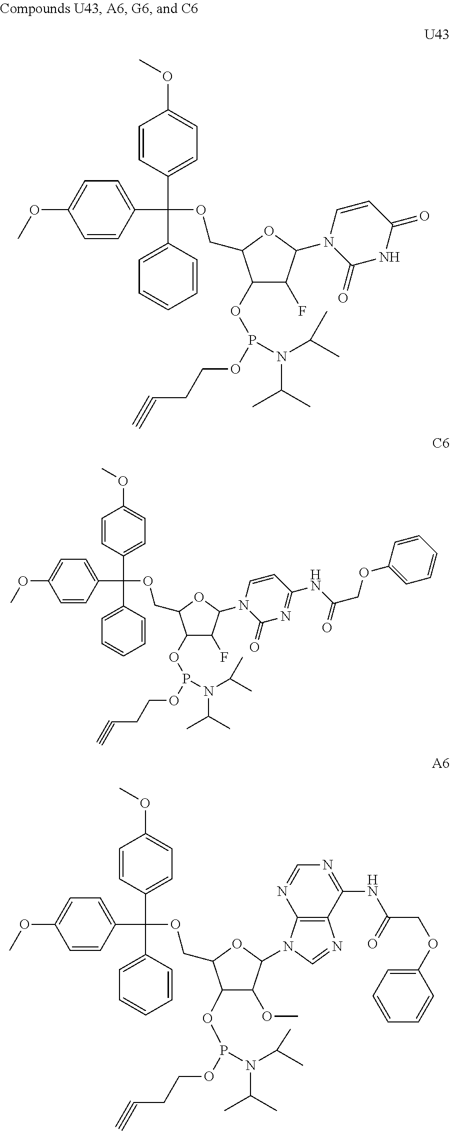 [SMILES: C#CCCOP(OC1C(COC(c2ccccc2)(c2ccc(OC)cc2)c2ccc(OC)cc2)OC(n2ccc(=O)[nH]c2=O)C1F)N(C(C)C)C(C)C ; C#CCCOP(OC1C(COC(c2ccccc2)(c2ccc(OC)cc2)c2ccc(OC)cc2)OC(n2ccc(NC(=O)COc3ccccc3)nc2=O)C1F)N(C(C)C)C(C)C ; C#CCCOP(OC1C(COC(c2ccccc2)(c2ccc(OC)cc2)c2ccc(OC)cc2)OC(n2cnc3c(NC(=O)COc4ccccc4)ncnc32)C1OC)N(C(C)C)C(C)C]

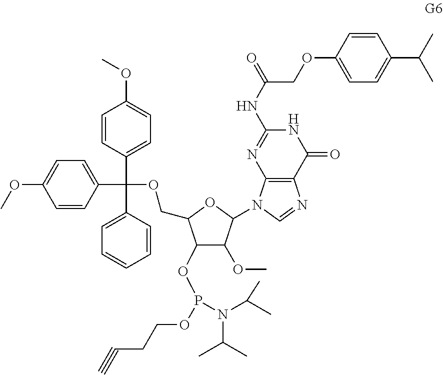 [SMILES: C#CCCOP(OC1C(COC(c2ccccc2)(c2ccc(OC)cc2)c2ccc(OC)cc2)OC(n2cnc3c(=O)[nH]c(NC(=O)COc4ccc(C(C)C)cc4)nc32)C1OC)N(C(C)C)C(C)C]

Compounds U43, A6, G6, and C6 were prepared according to methods known in the art from 3-butyn-1-ol, bis-(N,N-diisopropylamino)-chlorophosphine, and the corresponding protected nucleoside.

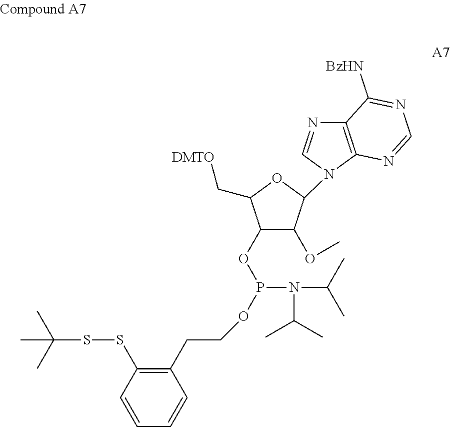

To a −78° C. cooled solution of 5′-O-(4,4′-dimethoxytrityl)-2′-O-methyl-adenosine (n-Bz) (14.24 g, 20.7 mmol) and N,N-diisopropylethylamine (4.0 mL, 22.7 mmol) in 100.0 mL of dry dichloromethane was added dropwise a solution of bis-(N,N-diisopropylamino)-chlorophosphine (6.07 g, 22.7 mmol) in 20.0 mL of dichloromethane under argon atmosphere. The reaction mixture was allowed to warm to room temperature, while stirring was maintained (1 hour). A solution of S61 (5.0 g, 20.7 mmol) in 15.0 mL of dry dichloromethane was added, the resulting mixture was stirred for 10 minutes, at which time a 0.25M acetonitrile solution of ETT (50.0 mL, 12.42 mmol) was added dropwise. The reaction mixture was further stirred for 16 hours at room temperature. The crude mixture was diluted with 200 mL of dichloromethane, washed sequentially with saturated NaHCO₃ solution (50 mL) and brine (50 mL), and dried over anhydrous Na₂SO₄. The solvent was evaporated in vacuo, and the crude mixture was purified by silica gel column chromatography using ethyl acetate/hexane solvent system (0-30% gradient on Combi Flash Rf Instrument) to give 8.7 g (40%) of product A7 (diastereomeric mixture) as white powder. ESI MS for C₅₇H₆₇N₆O₈PS₂ Calculated 1059.28, Observed 1057.9 [M−H]⁺. ³¹P NMR (202 MHz, CDCl₃): δ154.8, 154.0.

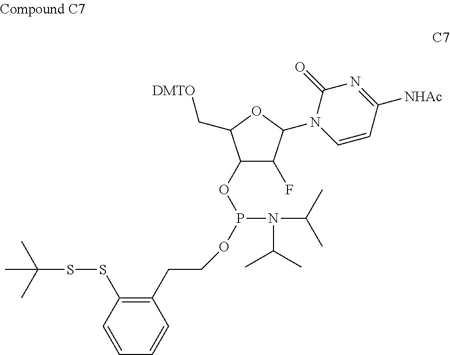

Compound C7 can be prepared using the protocol reported herein (e.g., the protocol described for A7).

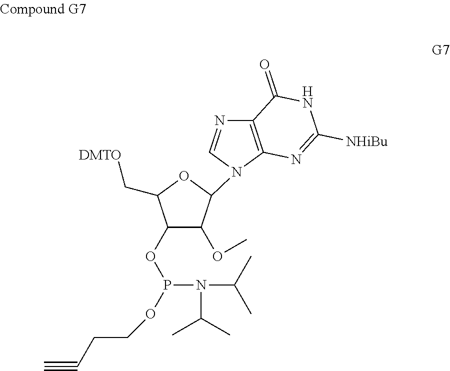

To a −78° C. cooled solution of but-3-yn-1-ol (0.52 g, 7.46 mmol) and N,N-diisopropylethylamine (1.35 mL, 7.78 mmol) in 15.0 mL of dry dichloromethane was added dropwise a solution of bis-(N,N-diisopropylamino)-chlorophosphine (2.07 g, 7.78 mmol) in 5.0 mL of dichloromethane under argon atmosphere. The reaction mixture was allowed to warm to room temperature, while stirring was maintained (1 hour). This solution was added dropwise to a dichloromethane (15 mL) suspension of 5′-O-(4,4′-dimethoxytrityl)-2′-O-methyl-Guanosine (iBu) (2.5 g, 3.73 mmol) and diisoproprylammonium tetrazolide (1.28 g, 7.46 mmol), and stirred for 16 hours at room temperature. The reaction mixture was diluted with 15 mL of dichloromethane and washed sequentially with saturated NaHCO₃ solution (10 mL) and brine (10 mL), then dried over anhydrous Na₂SO₄. The solvent was evaporated in vacuo, and the crude mixture was purified by silica gel column chromatography using ethyl acetate/hexane solvent system (0-60% gradient on Combi Flash Rf Instrument) to give 2.1 g (65%) of product G7 (diastereomeric mixture) as white powder. ESI MS for C₄₆H₅₇N₆O₉P Calculated 868.95, Observed 868.0 [M−H]⁺; ³¹P NMR (202 MHz, CDCl₃): δ155.4, 154.5.

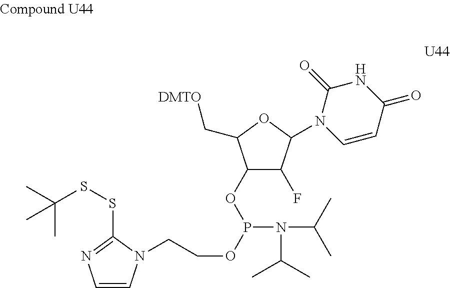

U44 was prepared according to the procedure described for compound U24. ESI MS for C₄₅H₅₇FN₅O₈PS₂, calculated 910.1, observed 910.7 (M+1); ³¹P NMR (202 MHz, CDCl₃): δ151.70 (d, J 8.1 Hz), 150.90 (d, J 9.5 Hz)

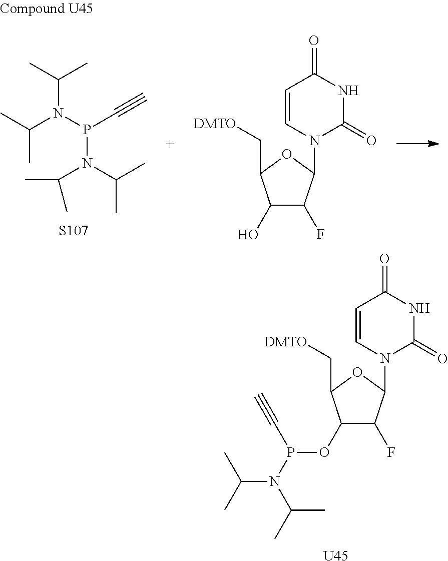

To a solution of S107 (1.28 g, 5.0 mmol) in 20 mL of dry dichloromethane was slowly added a solution of 5′-O-(4,4′-Dimethoxytrityl)-2′-F-Uridine (2.74 g, 5.0 mmol) and 1H-tetrazole (13.3 mL, 0.45 M in, 6.0 mmol) in 10 mL of dichloromethane under argon atmosphere and stirred for 1 hour. Triethylamine (50 μL) was slowly added to neutralize the reaction mixture, volatiles evaporated in vacuo and the crude mixture was purified by silica gel column chromatography using ethyl acetate/hexane solvent system (20-70% gradient on Combi Flash Rf Instrument) to give U45 as white powder (2.63 g, 75%). ESI MS for C₃₈H₄₇FN₃O₇P; calculated 703.7, observed 702.8 (M-1); ³¹P NMR (202 MHz, CDCl₃): δ109.65 (d, J 5.1 Hz), 106.24 (d, J 10.9 Hz).

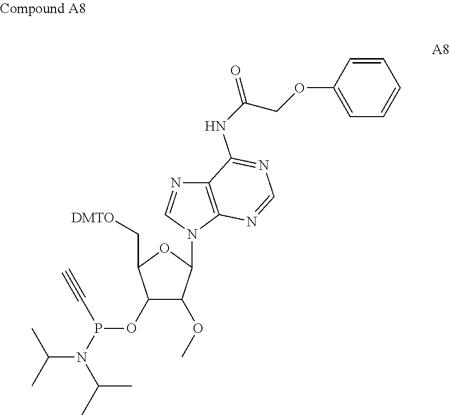

A8 was prepared using the same protocol as described for U45. ESI MS for C₄₈H₅₃N₆O₈P; calculated 872.9, observed 873.7 (M+1); ³¹P NMR (202 MHz, CDCl₃): δ106.37 (s), 105.97 (s).

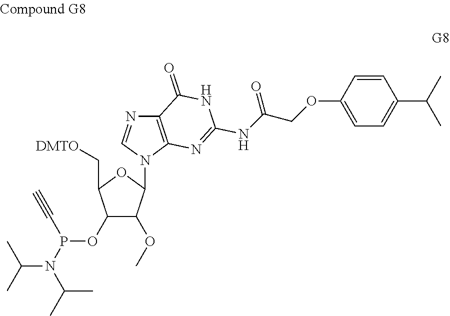

G8 was prepared using the same protocol as described for U45. ESI MS for C₅₁H₅₉N₆O₉P; calculated 931.0, observed 930.0 (M-1); ³¹P NMR (202 MHz, CDCl₃): δ106.57 (s), 105.27 (s).

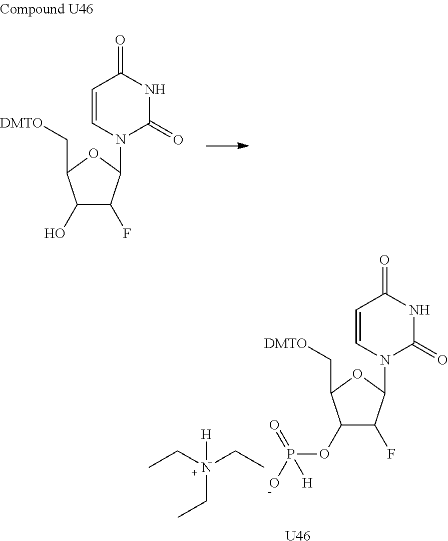

Phosphorous acid (1.64 g, 20.0 mmol) was co-evaporated three times with anhydrous pyridine (5 mL) and then dissolved in 10 mL of anhydrous pyridine upon heating. To this mixture was added 5′-O-(4,4′-Dimethoxytrityl)-2′-F-Uridine (1.10 g, 2.0 mmol), stirred for 10 min, cooled to 0° C. and then pivaloyl chloride (1.23 mL, 10.0 mmol) was slowly added. The mixture was warmed to room temperature and stirred overnight. The reaction was quenched with triethylammonium bicarbonate buffer (5 mL, 1M) followed by diluting with ethyl acetate (30 mL). After extraction with ethyl acetate (3×20 mL), the combined organic layers were washed with triethylammonium bicarbonate buffer (5 mL, 0.5M) and dried over anhydrous sodium sulfate. The volatiles removed under vacuo to afford a residue, which was subjected to flash silica gel column purification on ISCO companion (10% methanol/dichloromethane, containing 1% triethylamine) to give 0.96 g (67%) of U46 as white solid. ³¹P NMR (202 MHz, CDCl₃): δ9.08 (s).

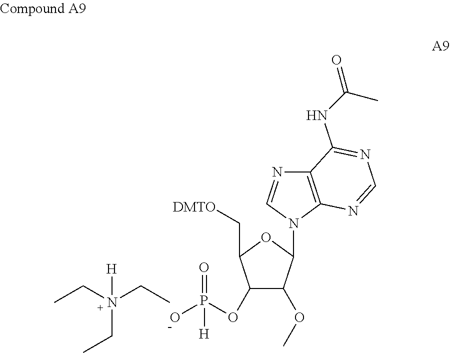

A9 was prepared using the protocol described for compound U46. ³¹P NMR (202 MHz, CDCl₃): δ4.33 (s), 3.51 (s).

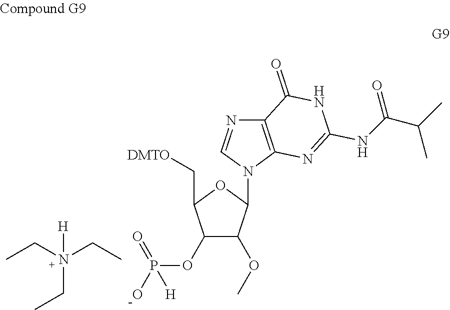

G9 was prepared using the protocol described for compound U46. ³¹P NMR (202 MHz, CDCl₃): δ3.89 (s), 3.25 (s).

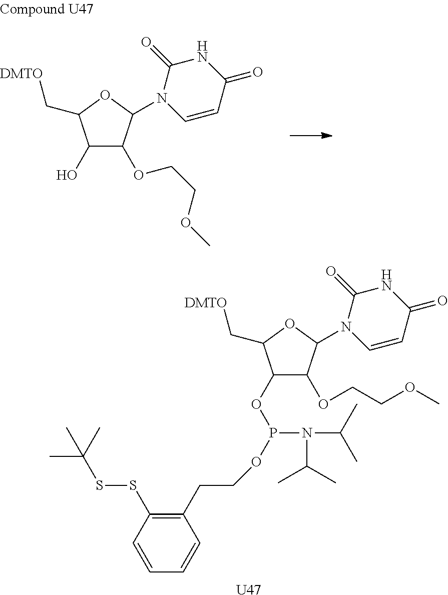

To a cooled solution (−78° C.) of 5′-O-(4,4′-dimethoxytrityl)-2′-MOE-Uridine (2.0 g, 3.3 mmol) and N,N-diisopropylethylamine (0.63 mL, 3.6 mmol) in 30 mL of dry dichloromethane under Argon was added dropwise a solution of bis-(N,N-diisopropylamino)-chlorophosphine (0.96 g, 3.6 mmol) in 10 mL of dichloromethane. The reaction mixture was allowed to warm to room temperature while stirring was maintained (1 hour). To this mixture, a solution of compound S61 (0.80 g, 3.3 mmol) in 5 mL of dry dichloromethane was added drop wise and stirred for 10 minutes before a suspension of diisoproprylammonium tetrazolide (DIAT, 0.56 g, 3.3 mmol) in 5 mL of dichloromethane was added portion-wise. The reaction mixture was further stirred for 16 hours at room temperature, diluted with 200 mL of dichloromethane and washed sequentially by saturated NaHCO₃ solution (40 mL) and brine (40 mL), then dried over anhydrous Na₂SO₄. The solvent was evaporated in vacuo and the crude mixture was purified by silica gel column chromatography using ethyl acetate/hexane solvent system (0-70% gradient on Combi Flash Rf Instrument) to give 1.28 g of product U47 as white powder (40% yield as diastereomeric mixture). ESI MS for C₅₁H₆₆N₃O₁₀PS₂, calculated 976.2, observed [M−H]⁺ 975.2; ³¹P NMR (202 MHz, CDCl₃): δ 148.9 (s), 148.6 (s).

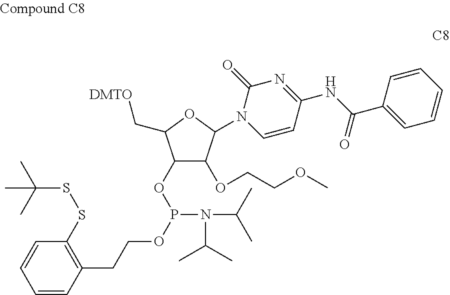

Compound C8 was prepared using the procedure described above (22% yield as diastereomeric mixture). ESI MS for C₅₈H₇₁N₄O₁₀PS₂, calculated 1079.3, observed [M−H]⁺ 1078.6; ³¹P NMR (202 MHz, CDCl₃): δ 149.0 (s), 147.8 (s).

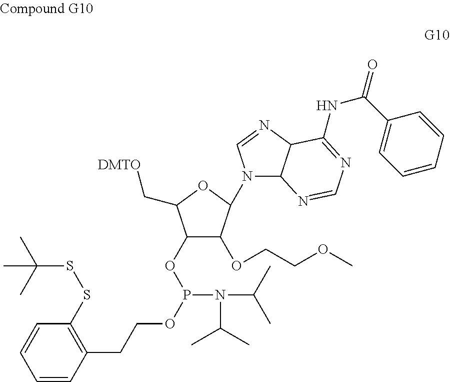

Compound G10 was prepared using the procedure described above (27% yield as diastereomeric mixture). ESI MS for C₅₉H₇₃N₆O₉PS₂ calculated 1105.4, observed [M−H]⁺ 1104.3; ³¹P NMR (202 MHz, CDCl₃): δ 149.4 (s), 148.8 (s).

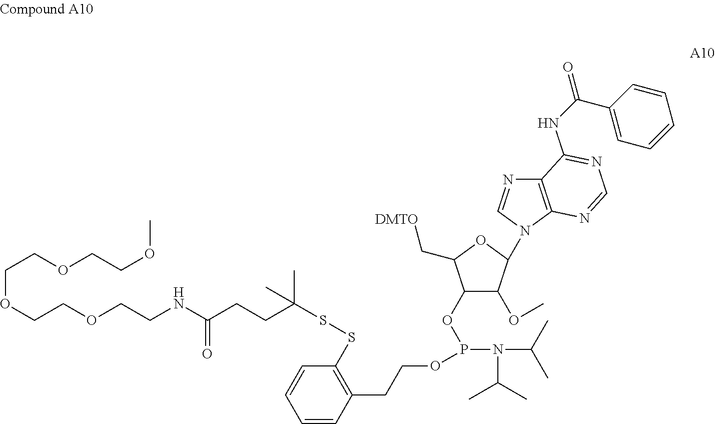

Compound A10 was prepared using the procedure described above (58% yield as diastereomeric mixture). ESI MS for C₆₈H₈₈N₇O₁₃PS₂ calculated 1306.6, observed [M+H]⁺ 1307.7; ³¹P NMR (202 MHz, CDCl₃): δ 154.7 (s), 154.1 (s).

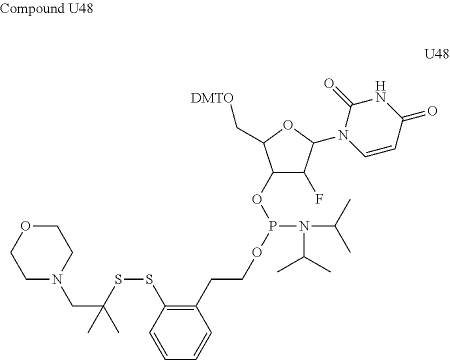

Compound U48 was prepared according to the procedure described above (54% yield as diastereomeric mixture). ESI MS for C₅₂H₆₆FN₄O₉PS₂ calculated 1005.2, observed [M−H]⁺ 1003.8; ³¹P NMR (202 MHz, CDCl₃) δ 154.7 (J _{P-F}=9.3 Hz), 154.6 (d, J_{P-F}=8.1 Hz).

The phosphoramidite monomers shown in Table 4 were synthesized using the standard synthetic procedures described herein.

TABLE 4

| Compound | | 31P NMR | Yield |
|---|---|---|---|
| # | Structure | (δ in ppm) | (%) |
| U49 | 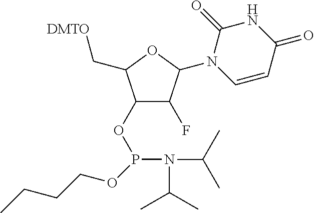 | 154.98 (d, J 8.08 Hz) 154.74 (d, J 8.08 Hz) | 54 |
| U50 | 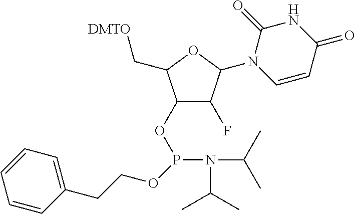 | 154.84 (d, J 12.12 Hz) 154.50 (d, J 8.08 Hz) | 53 |
| C9 | 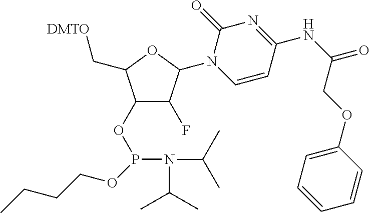 | 154.80 (d, J 8.08 Hz) 154.71 (d, J 6.06 Hz) | 51 |
| A11 | 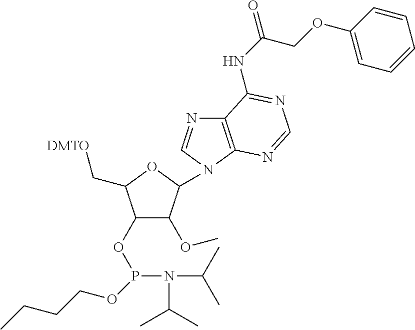 | 154.79 (s) 154.01 (s) | 34 |
| G11 | 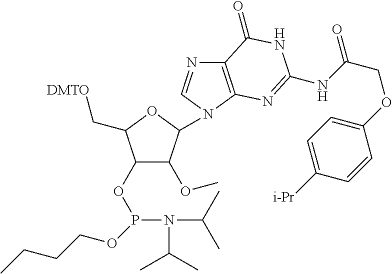 | 154.91 (s) 154.36 (s) | 55 |
| U51 | 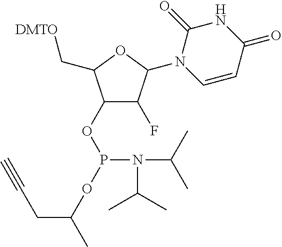 | 154.6 (d, J 6.6 Hz) 154.5 (d, J 8.5 Hz) 154.2 (d, J 9.3 Hz) 152.8 (d, J 10.1 Hz) | 60 |
| U43 | 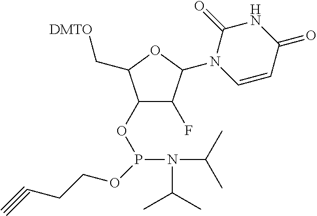 | 155.27 (d, J 6.06 Hz) 155.05 (d, J 8.08 Hz) | 50 |
| U52 | 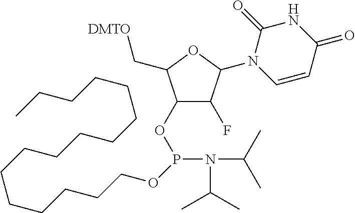 | 155.17 (d, J 8.08 Hz) 154.67 (d, J 10.1 Hz) | 48 |
| U53 | 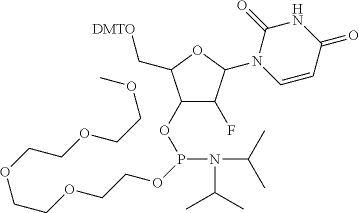 | 155.83 (d, J 6.06 Hz) 155.34 (d, J 10.1 Hz) | 55 |
| A12 | 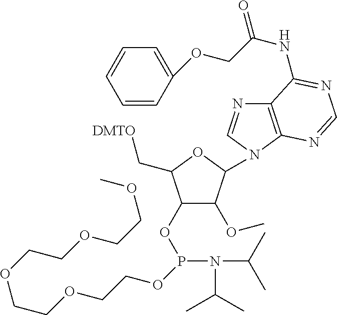 | 155.85 (s) 155.09 (s) | 25 |
| G6 | 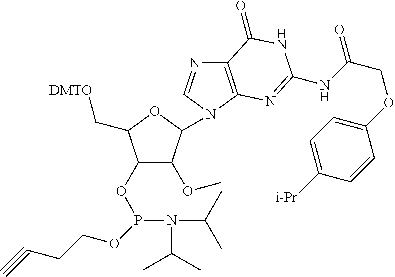 | 155.29 (s) 154.85 (s) | 29 |
| G12 | 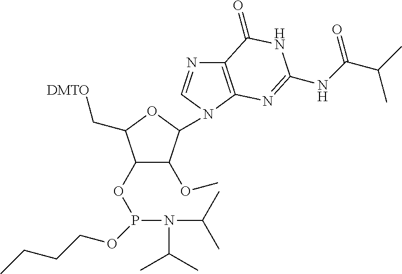 | 154.94 (s) 154.05 (s) | 18 |
| A13 | 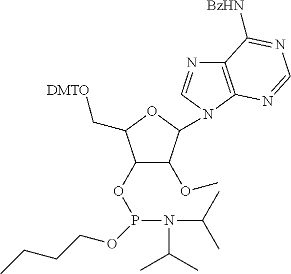 | 154.81 (s) 153.99 (s) | 55 |
| A14 | 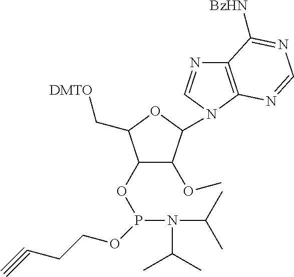 | 150.24 (s) 149.63 (s) | 39 |
| C6 | 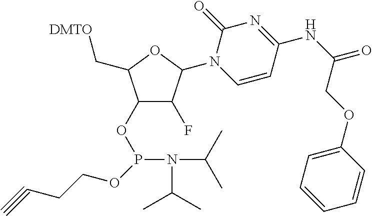 | 155.18 (d, J 6.06 Hz) 154.79 (d, J 8.08 Hz) | 55 |
| A6 | 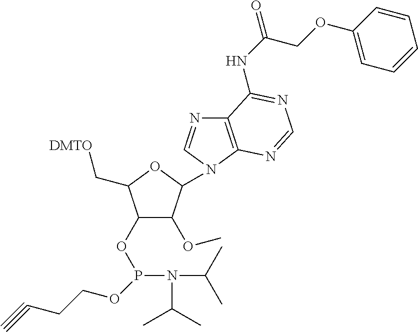 | 155.20 (s) 154.60 (s) | 56 |
| A15 | 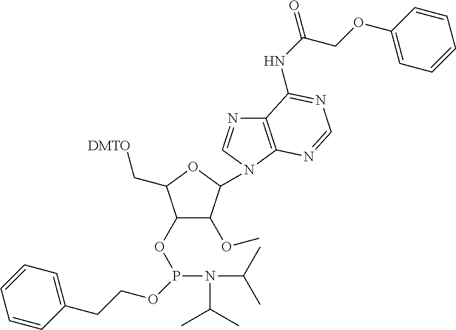 | 155.4 (s) 154.0 (s) | 80 |
| U54 | 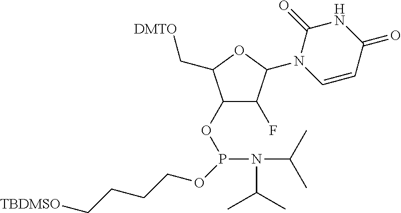 | 155.4 (d, J 7.9 Hz) 154.7 (d, J 9.7 Hz) | 54 |

The synthetic methods described herein may be used to prepare other phosphoramidite monomers that may be used in the preparation of the polynucleotides of the invention, for example:

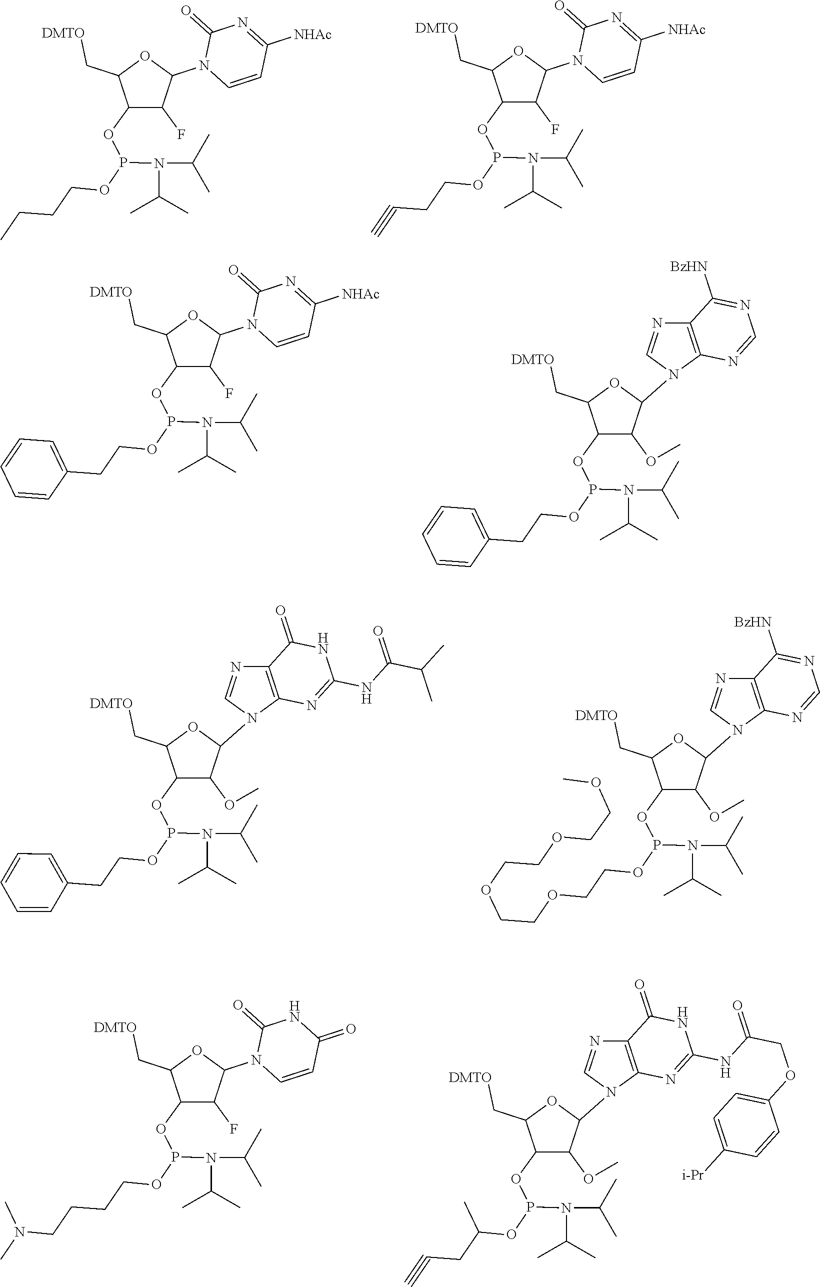

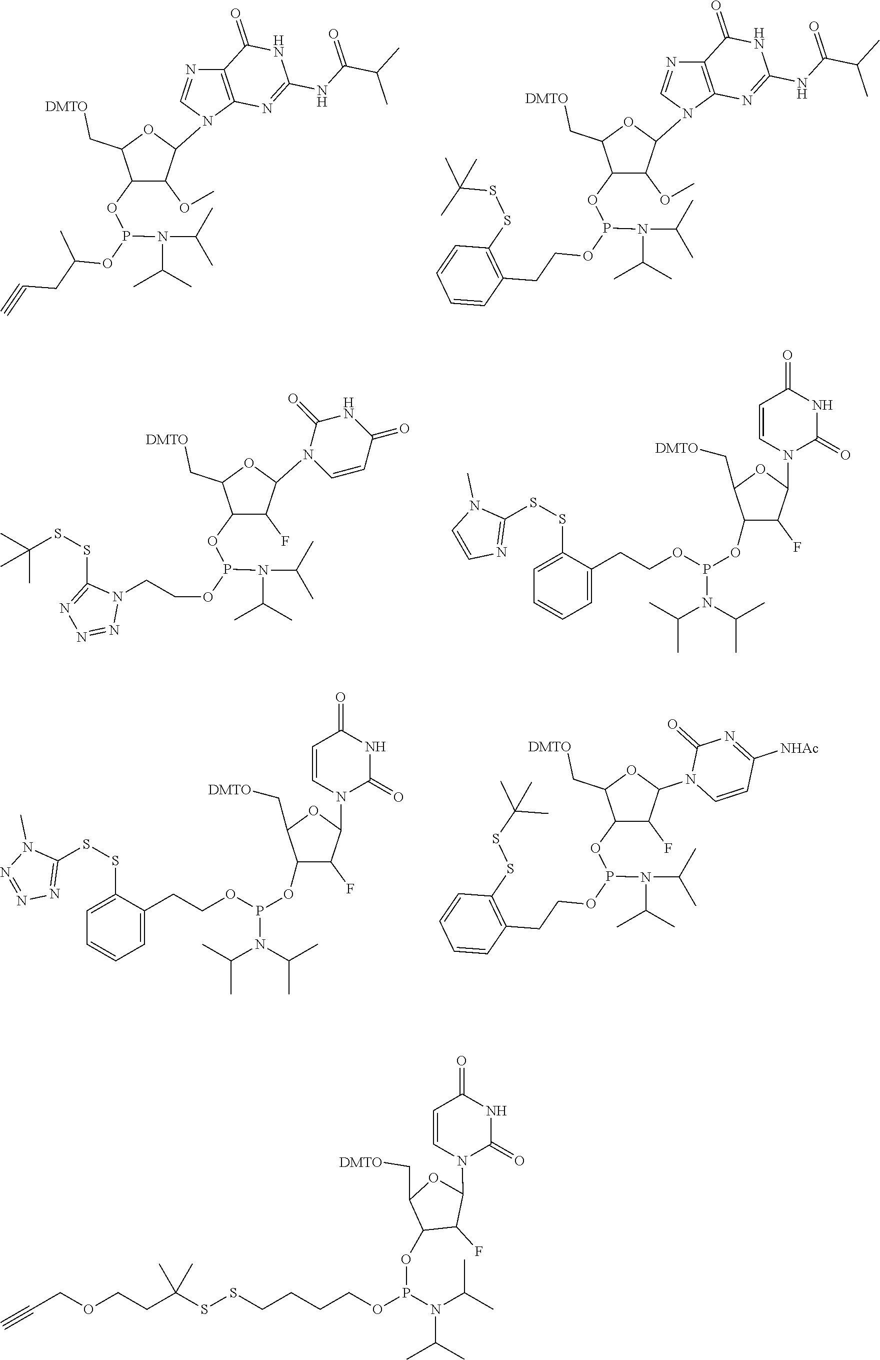

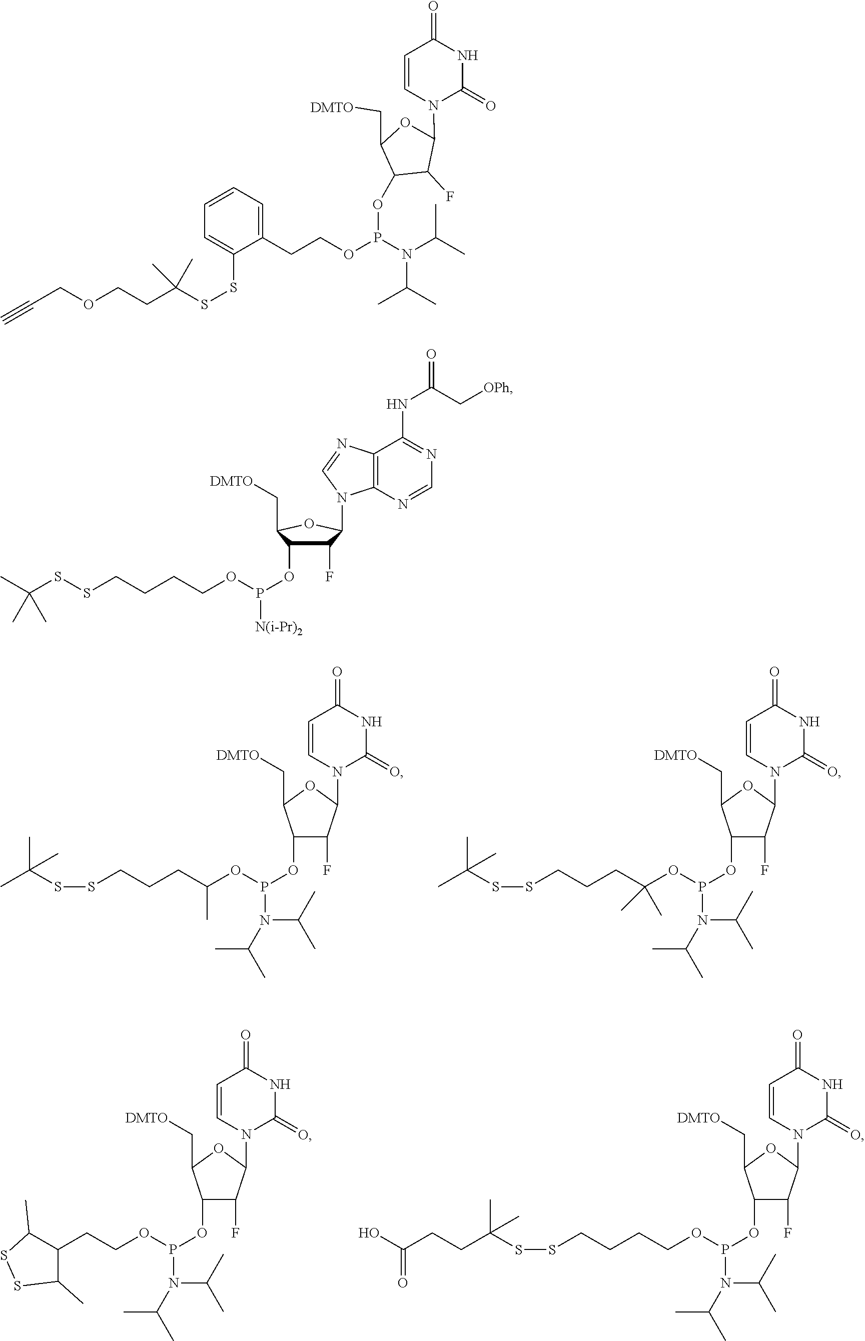

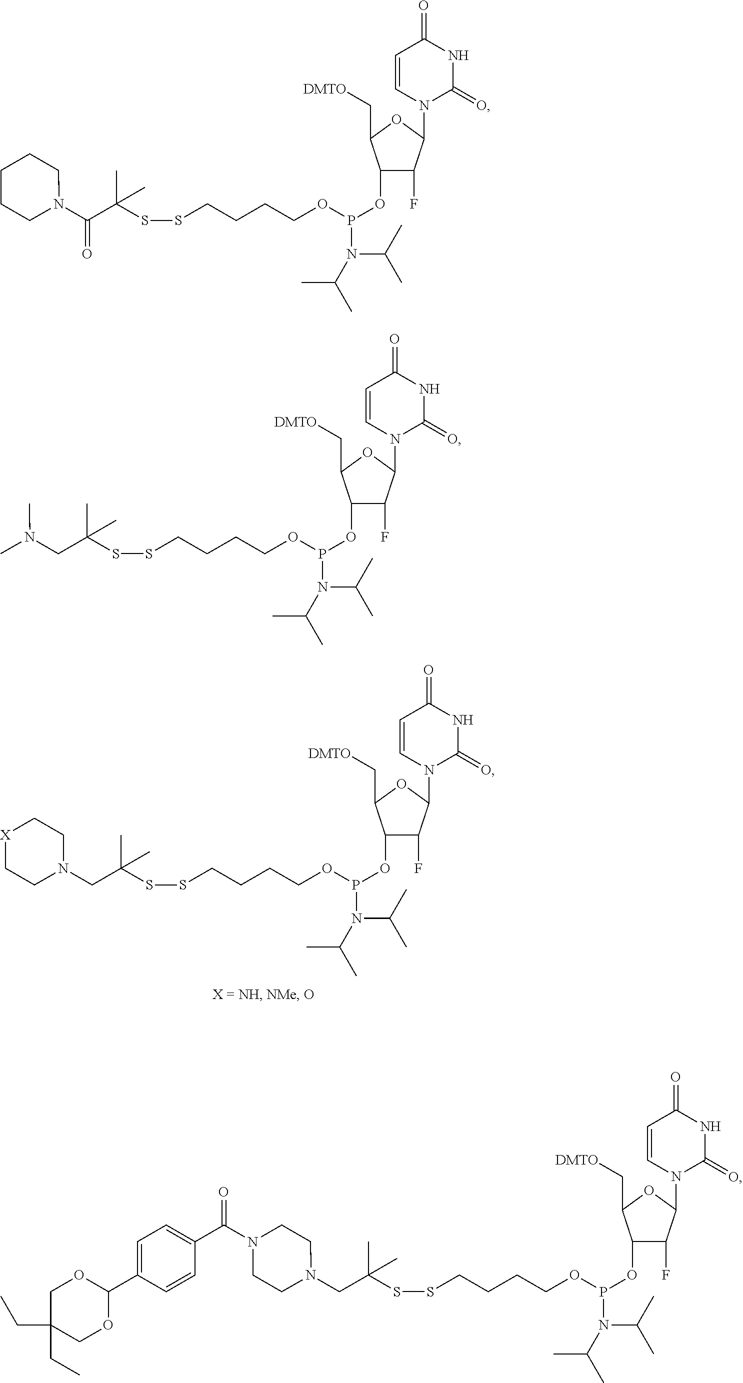

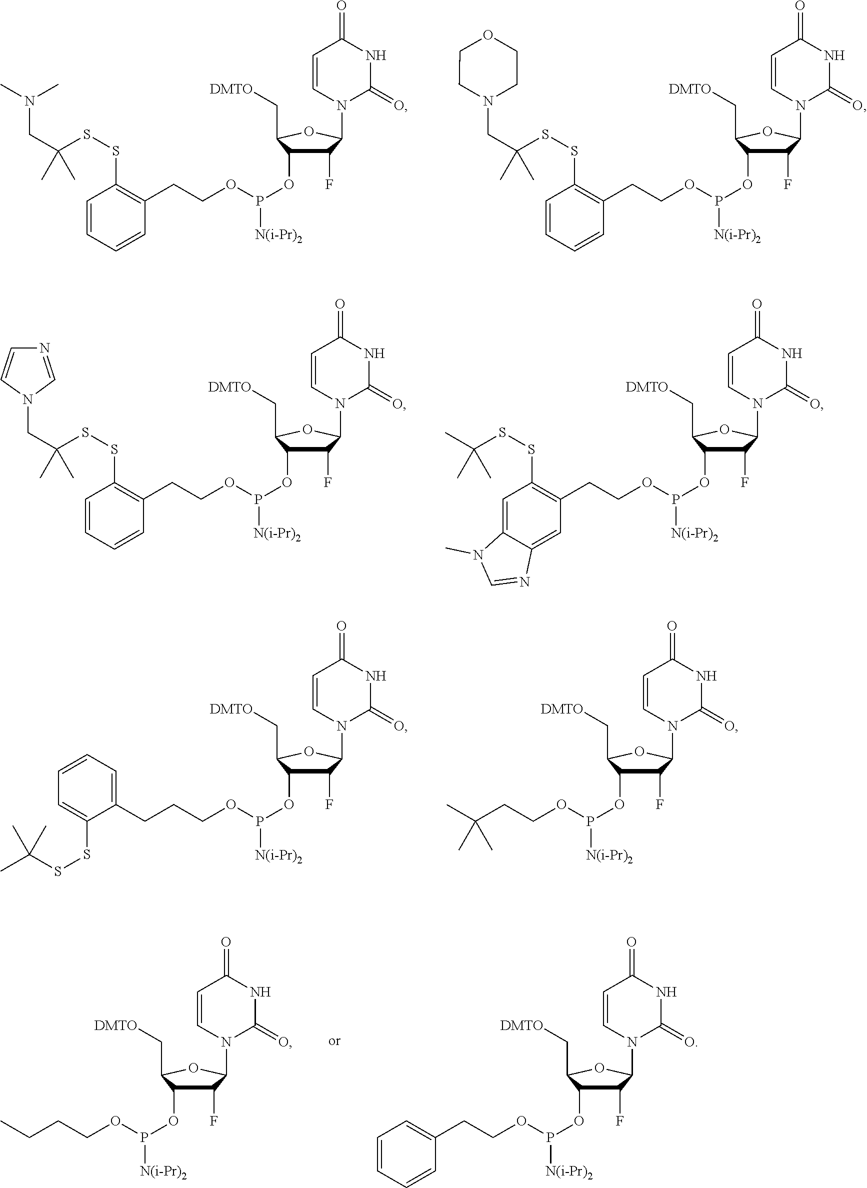

Additionally, the following phosphoramidite monomers having targeting ligands such as mannose, GaINAc, etc. can be synthesized using the procedure described for M21. Similar approaches can be utilized for other small molecule/peptide targeting ligands, e.g. folate, PSMA, CPP, etc.

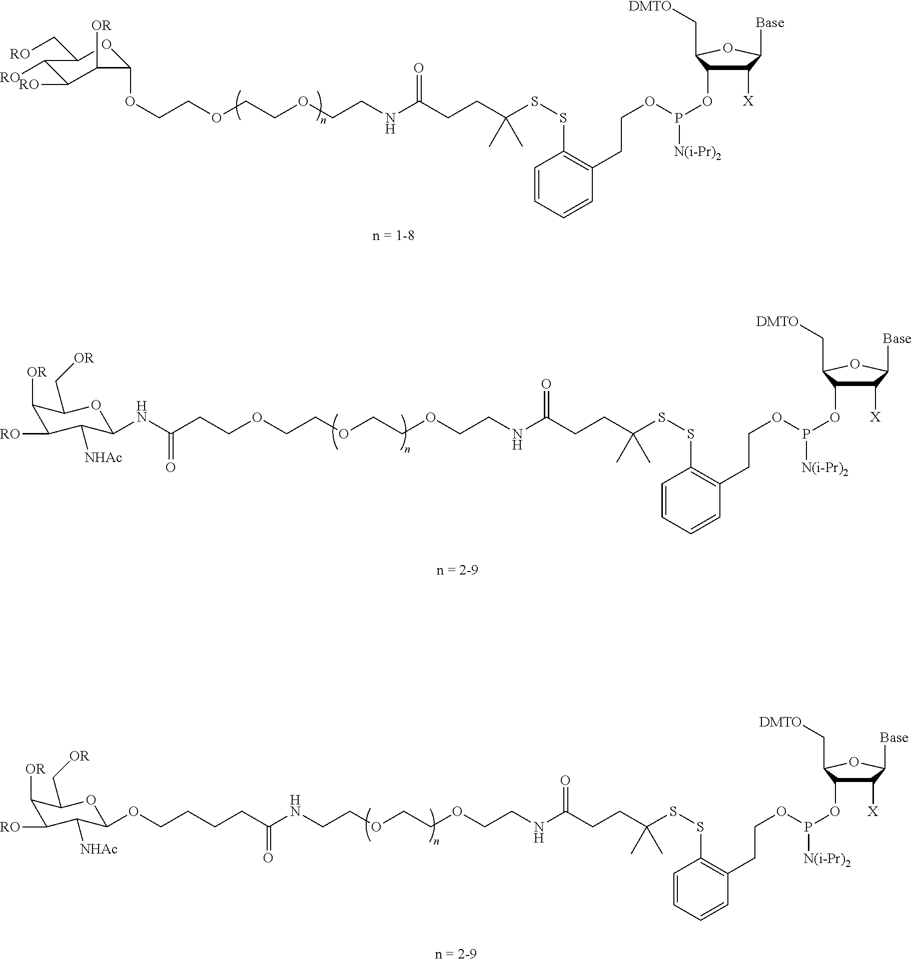

the scheme above, X can be F, OMe, 2-methoxyethyl (MOE), etc.; Base can be U, C, A, G; and R can be Ac, tert-butyldimethylsilyl (TBDMS), allyl, etc.

### Synthesis of Cell Penetrating Peptides (Protein Transduction Domains)

### Peptide Synthesis:

Synthesis: Rink amide polystyrene resin (0.080 g, 0.61 mmol/g) was added to the reaction vessel, swelled three times in dimethylformamide (5 volumes) for 7 min. each time with nitrogen bubbling and then drained. The assembly of the peptide was carried out using the following cycles and employing standard Fmoc chemistry:
Fmoc deprotection with 20% piperidine in dimethylformamide (DMF) 3×4 min;
Resin washed with DMF, 6×1 min;
Couplings used 5 eq. protected amino acid, 15 eq. N-methylmorpholine (NMM), and 5 eq. HCTU. After adding the coupling solution, the reaction was allowed to proceed for 2×20 min;
On completion of coupling, the resin was washed with DMF for 6×1 min;
For the final assembly step, the N-terminus was capped by adding 5 eq. of Fmoc-6-Hydrazinoicotinic Acid; 5 eq. HATU and 15 eq. NMM in DMF and mixing until the reaction was complete (around 1 hr), as confirmed by the Kaiser (ninhydrin) test. The Fmoc removed by 20% piperidine in DMF 3×4 min; and
The completed resin-bound peptide was washed three times with DMF, three times with dichloromethane (DCM) and then dried under vacuum.

Cleavage: The peptide was cleaved/deprotected from the resin using the following solution: trifluoroacetic acid/dithiothreitol/water/acetone/triisopropylsilane (10 ml, 90/3/2/3/2), with stirring for 2 hr. The resin was filtered through a medium frit, syringe filter and washed twice with neat trifluoroacetic acid (TFA). The filtrates were combined and the volume reduced to half by evaporation. The TFA solution was stirred and the crude peptide precipitated by the slow addition of 4 volumes of ice-cold ether. The precipitated crude peptide was collected by filtration.

Purification: The crude material was analyzed by LC/MS using a 15-75% B (A=0.1% trifluoroacetic acid/water; B=0.1% trifluoroacetic acid/acetonitrile) over 20 min using a Phenomenex Luna C₁₈ (100×4.6 mm 5p) column.

List of Cell Penentrating Peptides, Endosomolytic peptides, and certain targeting moieties synthesized is shown in Table 3.

### Synthesis of Targeting Ligands

### GaINAc (NAG) Ligand Synthesis:

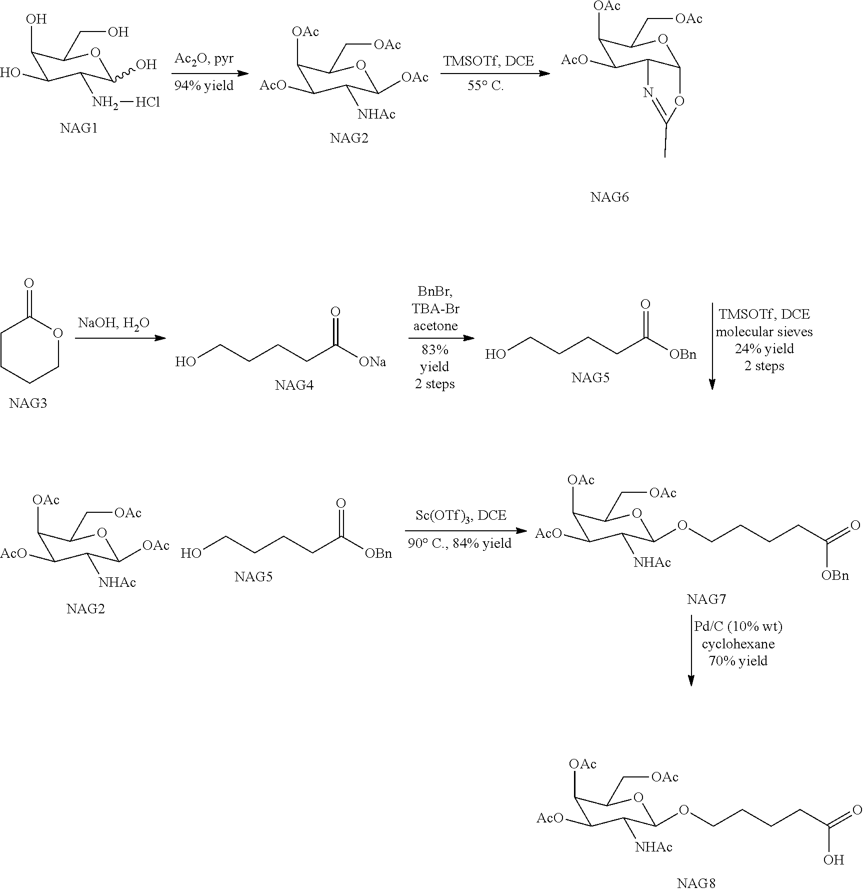

Preparation of D-galactosamine pentaacetate (NAG2). D-Galactosamine (25.0 g, 116 mmol) was suspended in anhydrous pyridine (250 mL) and cooled to 0° C. under an inert atmosphere. Acetic anhydride (120 mL, 1160 mmol) was added over the course of 2 h. After stirring overnight, the reaction mixture was concentrated in vacuo. Upon addition of methanol, a white solid precipitated and was collected by filtration to provide the desired product (42.1 g, 93% yield). ¹H NMR (CDCl₃, 500 MHz): δ 5.69 (d, 1H, J 9.0 Hz), 5.40 (m, 1H), 5.37 (d, 1H, J 3.0 Hz), 5.08 (dd, 1H, J 3.0 Hz, 11 Hz), 4.44 (dt, 1H, J 9.5 Hz, 11 Hz), 4.17 (dd, 1H, J 7.0 Hz, 11.5 Hz), 4.11 (dd, 1H, J 7.0 Hz, 11.5 Hz), 4.01 (t, 1H, J 7.0 Hz), 2.17 (s, 3H), 2.13 (s, 3H), 2.05 (s, 3H), 2.02 (s, 3H), 1.94 (s, 3H), 1.57 (s, 3H).

Preparation of benzyl 5-hydroxy pentanoate (NAG5). A solution of delta-valerolactone (10.0 g, 100 mmol) and NaOH (4.00 g, 100 mmol) in water (100 mL) was stirred overnight at 70° C. The reaction mixture was cooled to rt and concentrated in vacuo to give white solid NAG4. This solid was suspended in acetone (100 mL) and refluxed overnight with benzyl bromide (20.5 g, 120 mmol) and tetrabutylammonium bromide (1.61 g, 0.50 mmol). Acetone was removed in vacuo to afford an oily residue, which was dissolved in EtOAc and washed with sat. NaHCO₃ (aq.) and brine. The organic layer was dried over Na₂SO₄ and concentrated in vacuo to give NAG5 as oily product (17.1 g, 82% yield). ¹H NMR (CDCl₃, 500 MHz): δ 7.35 (m, 5H), 3.64 (q, 2H, J 6 Hz, 11.5 Hz), 2.41 (t, 2H, J 7.5 Hz), 1.75 (m, 2H), 1.60 (m, 2H), 1.44 (t, 1H, J 6 Hz).

Preparation of benzyloxycarbonylbutyl 2-deoxy 2-N-acetyl -3,4,6-tri-O-acetyl-β-D-galactopyranoside (NAG7)—Method A. Under an inert atmosphere, TMSOTf (8.56 g, 38.4 mmol) was added to a solution of NAG2 (10.0 g, 25.6 mmol) in DCE (100 mL) at ambient temperature. The mixture was stirred at 55° C. for 2 h, removed from heat, and stirred overnight. The reaction mixture was poured onto ice cold sat NaHCO₃ (aq.) and extracted with CH₂Cl₂. The organic layer was dried over Na₂SO₄ and concentrated in vacuo to give syrup NAG6. A solution NAG6 in DCE (60 mL) was charged with alcohol NAG5 (8.00 g, 38.4 mmol) and molecular sieves. The mixture was placed under an inert atmosphere, treated with TMSOTf (2.85 g, 12.8 mmol), and stirred overnight at rt. The mixture was poured over ice cold sat NaHCO₃ (aq.) and extracted with CH₂C1₂. The organic layer was dried over Na₂SO₄ and concentrated in vacuo to give syrup. This crude material was purified via SiO₂ gel chromatography to afford glycoside NAG7 (3.3 g, 24% yield). ¹H NMR (CDCl₃, 500 MHz): δ 7.35 (m, 5H), 5.98 (d, 1H, J 7.0 Hz), 5.57 (m, 1H), 5.34 (d, 1H, J 3.0 Hz), 5.25 (dd, 1H, J 3.0 Hz, 11 Hz), 5.10 (s, 2H), 4.63 (d, 1H, J 8.5 Hz), 4.11 (m, 2H), 3.95 (m, 1H), 3.88 (m, 2H), 3.49 (m, 1H), 2.37 (m, 2H), 2.13 (s, 3H), 2.03 (s, 3H), 1.99 (s, 3H), 1.90 (s, 3H), 1.70 (m, 2H), 1.61 (m, 2H).

Preparation of benzyloxycarbonylbutyl 2-deoxy 2-N-acetyl -3,4,6-tri-O-acetyl-β-D-galactopyranoside (NAG7)—Method B. To a solution of NAG2 (5.00 g, 12.8 mmol) and alcohol NAG5 (5.33 g, 25.6 mmol) in DCE (50 mL) was added Sc(OTf)₃ (0.44 g, 0.90 mmol) in one portion. The mixture was placed under an inert atmosphere and refluxed for 3 h. Upon cooling the mixture was diluted with CH₂Cl₂, washed with sat. NaHCO₃ (aq.), dried over MgSO₄, and concentrated in vacuo. Purification via SiO₂ gel chromatography afforded glycoside NAG7 (5.53 g, 80% yield).

Preparation of carboxybutyl 2-deoxy 2-N-acetyl -3,4,6-tri-O-acetyl-β-D-galactopyranoside (NAG8). A solution of glycoside NAG7 (1.50 g, 2.41 mmol) in EtOH (25 mL) was degassed by application of vacuum and backfilling with argon. The palladium catalyst (10% wt. on activated carbon, 0.50 g) was added in one portion, and the mixture was degassed by application of vacuum and backfilling with argon. To the heterogeneous mixture was added cyclohexene (25 mL) and refluxed for 6 h. Upon cooling the catalyst was removed by filtration, and the mother liquor was concentrated in vacuo. The crude was purified via SiO₂ gel chromatography to afford a white foam NAG8 (0.76 g, 70% yield). 1H NMR (CDCl₃, 500 MHz): δ 5.72 (d, 1H, J 8.5 Hz), 5.35 (d, 1H, J 3.5 Hz), 5.26 (dd, 1H, J 3.5 Hz, 11.5 Hz), 4.67 (d, 1H, J 8.5 Hz), 4.17 (dd, 1H, J 6.5 Hz, 11.5 Hz), 4.12 (dd, 1H, 6.5 Hz, 11.5 Hz), 4.00 (dt, 1H, J 8.5 Hz, 11.5 Hz), 3.92 (m, 2H), 3.53 (m, 1H), 2.39 (m, 2H), 2.15 (s, 3H), 2.05 (s, 3H), 2.01 (s, 3H), 1.97 (s, 3H), 1.71 (m, 2H), 1.65 (m, 2H).

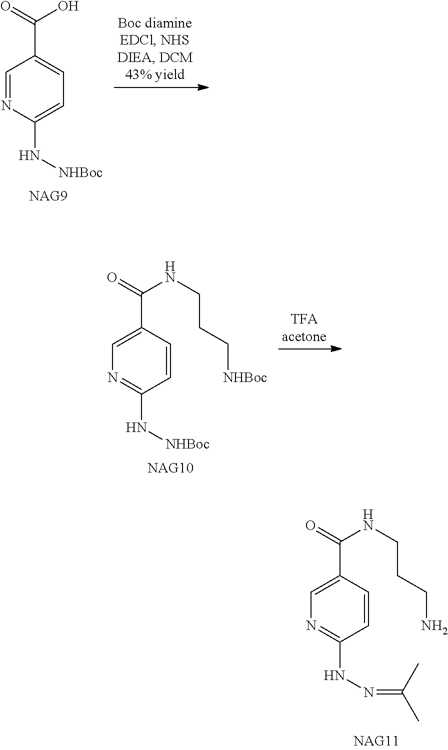 [SMILES: CC(C)(C)OC(=O)NNc1ccc(C(=O)O)cn1 ; CC(C)(C)OC(=O)NCCCNC(=O)c1ccc(NNC(=O)OC(C)(C)C)nc1 ; CC(C)=NNc1ccc(C(=O)NCCCN)cn1]

Preparation of aminopropyl 6-hydrazinonicotamide acetone hydrazone (NAG11). Boc 6-hydrazinonicotinic acid (520 mg, 2.1 mmol) in DCM (20 mL) was treated with EDCl (440 mg, 2.3 mmol), N-hydroxysuccinimide (NHS; 260 mg, 2.3 mmol), Boc-diamine (650 mg, 2.6 mmol), and DIEA (1.1 mL, 6.2 mmol) for 3 h. The reaction mixture was concentrated in vacuo and purified by silica gel chromatography to afford NAG10 (364 mg, 43% yield). ¹H NMR (CDCl₃, 500 MHz): δ 8.55 (br, 1H), 7.93 (d, 2H, J 7.5 Hz), 7.45 (br, 1H), 7.12 (br, 1H), 6.62 (d, 1H, J 8.5 Hz), 5.17 (br, 1H), 3.42 (m, 2H), 3.13 (m, 2H), 1.65 (m, 2H), 1.41 (s, 18H). The HyNic acetone hydrazone was formed through treatment of NAG10 (160 mg, 0.4 mmol) with TFA (9 mL) and acetone (1 mL) for 1 h. The reaction mixture was concentrated in vacuo and placed on the high vacuum to afford NAG11.

### Synthesis of Trivalent GaINAc-HyNic

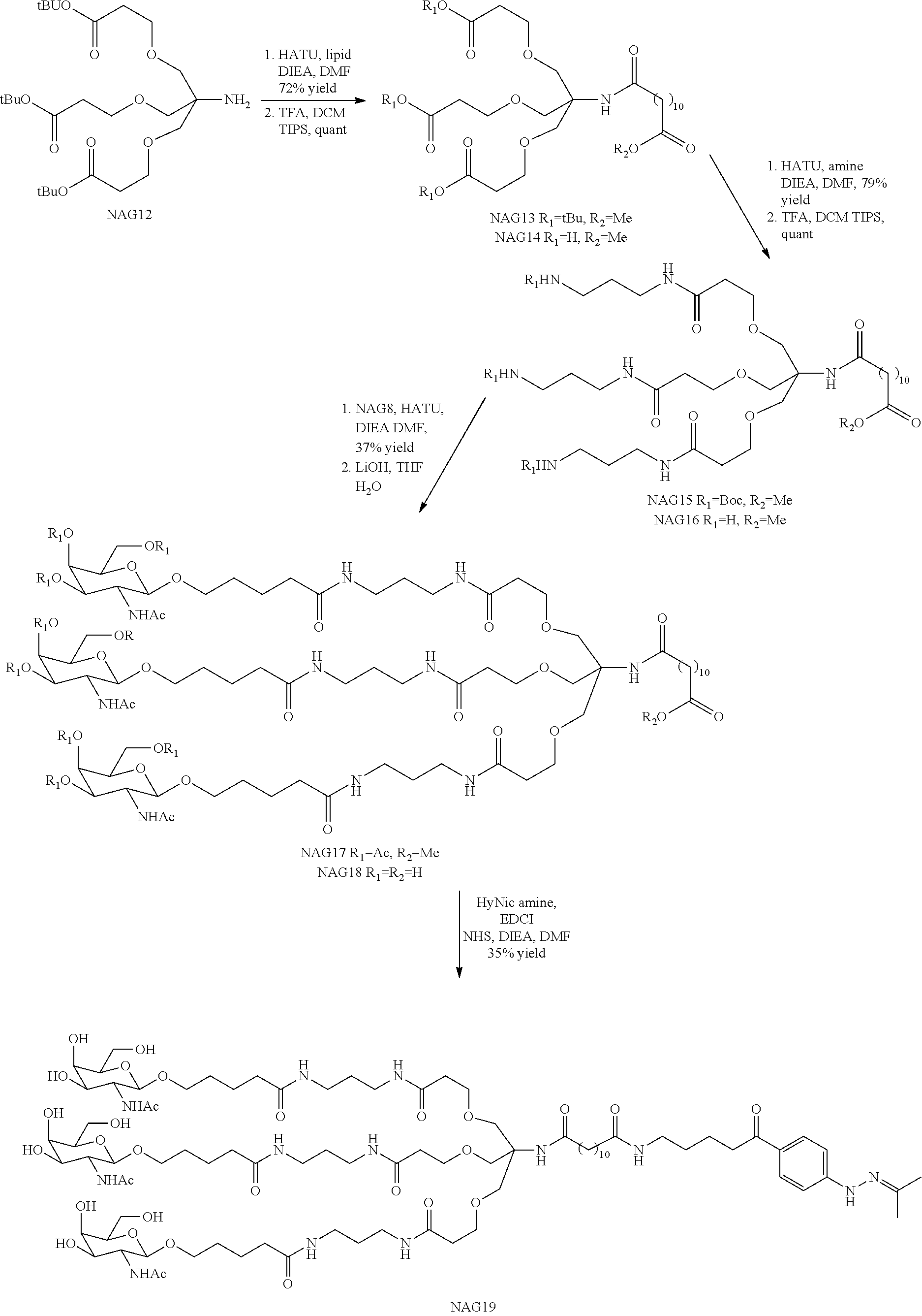

Preparation of tris-(carboxyethoxymethyl)-methylamido-dodecaneioate methyl ester (NAG4) To a solution of dodecanedioic acid methyl ester (211 mg, 0.42 mmol) activated with HATU (122 mg, 0.50 mmol) and DIEA (218 μL, 1.25 mmol) in DMF (2 mL) was added tris linker NAG12. After 1 h, the reaction mixture was concentrated in vacuo and purified by SiO₂ gel chromatography to afford NAG13 (214 mg, 70% yield). MALDI-TOF mass calcd C₃₈H₆₉NO₁₂: 731.48, Found: 755.10 [M+Na]. Tris t-butyl ester NAG13 was hydrolyzed with a TFA:TIPS:DCM (9:0.25:1) cocktail (10.25 mL) for 4 h and concentrated in vacuo to give tris acid NAG14. MALDI-TOF mass calcd C₂₆H₄₅NO₁₂: 563.29, Found: 565.33 [M+H].

Preparation of tris-(aminopropamido-ethoxymethyl)-methylamido-dodecanedioate methyl ester (NAG16). To a solution of tris acid NAG14 (230 mg, 0.41 mmol) activated with HATU (557 mg, 1.35 mmol) and DIEA (470 μL, 2.70 mmol) in DMF (4 mL) was added monoBoc 1,3-diaminopropane (250 mg, 1.44 mmol). After 1 h, the reaction was concentrated in vacuo and purified by SiO₂ gel chromatography to afford NAG15 (335 mg, 79% yield). MALDI-TOF mass calcd C₅₀H₉₃N₇O₁₅: 1031.67, Found: 1056.40 [M+Na]. Tris Boc linker NAG15 was treated with a TFA:TIPS:DCM (9:0.25:1) cocktail (10.25 mL) for 1 h and concentrated in vacuo to give tris amine NAG16. MALDI-TOF mass calcd C₃₅H₆₉N₇O₉: 731.51, Found: 733.18 [M+H].

Preparation of tris-GaINAc (NAG18): Monosaccharide NAG8 (192 mg, 0.43 mmol) was treated with HATU (163 mg, 0.43 mmol) and DIEA (150 μL, 0.86 mmol) in DMF (2 mL). After 30 min, a solution of NAG16 (80 mg, 0.11 mmol) in DMF (1 mL) was added, and the mixture was stirred for 1 h. The crude mixture was purified by SiO₂ gel chromatography to afford NAG17 (82 mg, 37% yield). Mass calcd C₉₂H₁₅₀N₁₀O₃₉: 2019.00, Found: 2041.85 [M+Na]. The peracetylated trimer GaINAc (82 mg, 0.04 mmol) was hydrolyzed upon treatment with LiOH—H₂O (34 mg, 0.81 mmol) in a THF:H₂O (3:1) solution (8 mL) to afford NAG18. MALDI-TOF mass calcd C₇₃H₁₃₀N₁₀O₃₀: 1626.89, Found: 1634.52 [M+Li].

Preparation of HyNic trimer GalNAc (NAG19). A solution of GalNAc trimer NAG18 (32 mg, 0.02 mmol) and HyNic amine NAG11 (20.0 mg, 0.08 mmol) in DMF (1 mL) was treated with EDCl (16.2 mg, 0.08 mmol), NHS (2.5 mg, 0.02 mmol), and DIEA (28 μL, 0.16 mmol) and stirred for 4 h. Upon concentration in vacuo, the crude was dissolved in DMSO and purified by RP-HPLC to afford NAG19 (12.6 mg, 35% yield). MALDI-TOF mass calcd C₈₅H₁₄₇N₁₅O₃₀: 1858.04, Found: 1859.83 [M+H].

### Synthesis of Trivalent GaINAc Azide

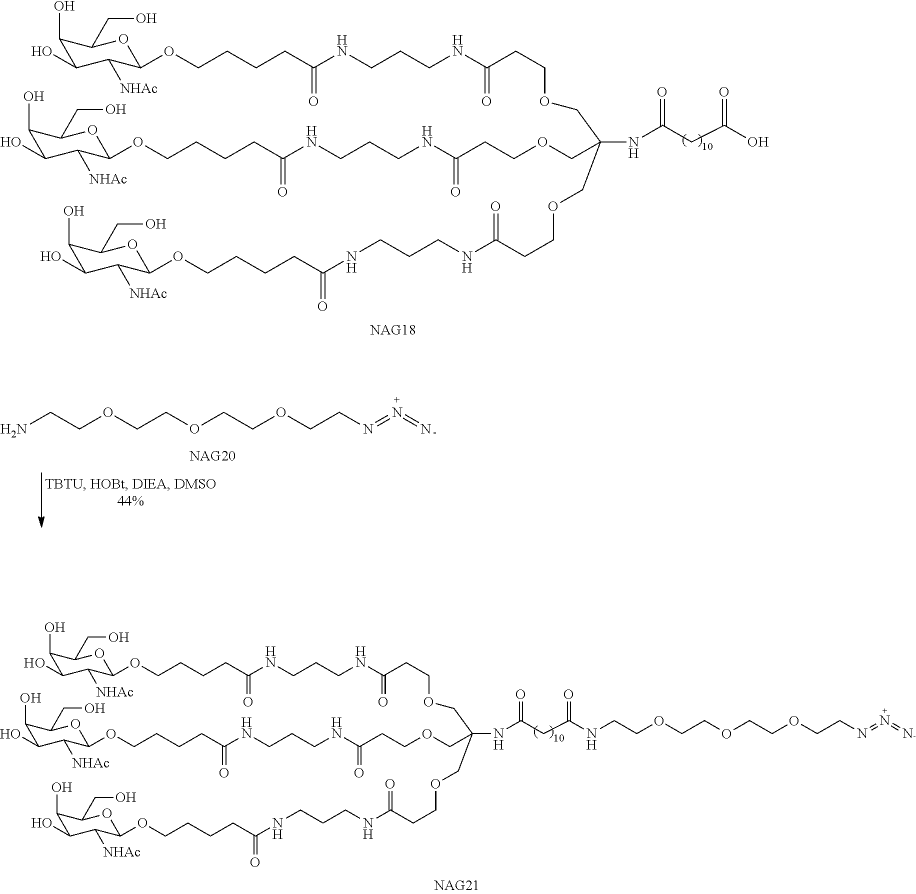

Preparation of azido-Peg₃-trimer GalNAc (NAG21). GalNAc trimer carboxylic acid NAG18 (60 mg, 0.03 mmol), azido-Peg₃-amine NAG20 (45.6 mg, 0.21 mmol), TBTU (23.8 mg, 0.07 mmol), HOBt (11.5 mg, 0.03 mmol), and DIEA (34 uL) were dissolved in DMSO (0.5 mL) and stirred 2 h. The base was removed in vacuo, and the crude was purified by RP-HPLC to afford NAG21 (24 mg, 44%). AP-ESI+ Mass calcd C₈₁H₁₄₆N₁₄O₃₂: 1827.02, Found: 914.8 [M+2H]²⁺

### Synthesis of GaINAc Azide

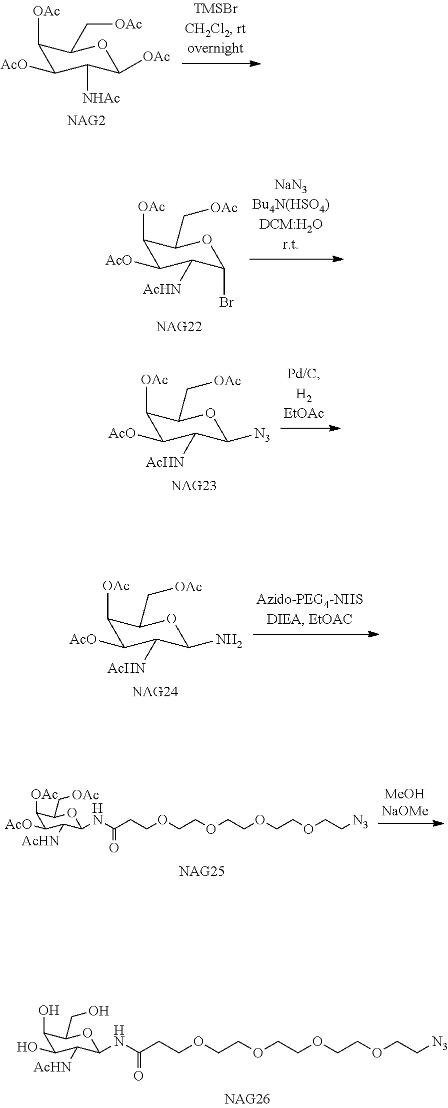 [SMILES: CC(=O)NC1C(OC(C)=O)OC(COC(C)=O)C(OC(C)=O)C1OC(C)=O ; CC(=O)NC1C(Br)OC(COC(C)=O)C(OC(C)=O)C1OC(C)=O ; CC(=O)NC1C(N=[N+]=[N-])OC(COC(C)=O)C(OC(C)=O)C1OC(C)=O ; CC(=O)NC1C(N)OC(COC(C)=O)C(OC(C)=O)C1OC(C)=O ; CC(=O)NC1C(NC(=O)CCOCCOCCOCCOCCN=[N+]=[N-])OC(COC(C)=O)C(OC(C)=O)C1OC(C)=O ; CC(=O)NC1C(NC(=O)CCOCCOCCOCCOCCN=[N+]=[N-])OC(CO)C(O)C1O]

Preparation of 1-bromo 2-deoxy-2-acetamido 3,4,6-tri-O-acetyl-β-D-galactopyranoside (NAG22).

To a D-galactosamine pentaacetate (NAG2, 10.0 g, 1 eq, 25.8 mmol) suspension in DCM (90 ml) at 0° C. in an ice bath under an argon balloon was added bromotrimethylsilane (4.1 ml, 1.2 eq, 31 mmol) dropwise with stirring. Ice bath was removed after 10 minutes, and the reaction was allowed to stir at room temperature overnight. The reaction progress was checked by TLC (Hanessian's stain) in 75% hexanes:ethyl acetate. The reaction mixture was concentrated in vacuo, azeotroped with cyclohexane (3×50 mL), dried under high vacuum overnight, and the resulting product was used as is.

Preparation of 1-azido 2-deoxy-2-acetamido 3,4,6-tri-O-acetyl-β-D-galactopyranoside (NAG23).

NAG22 (10.6 g, 1.0 eq, 25.8 mmol) was dissolved in DCM (100 ml). To this solution was added sodium azide (4.86 g, 2.9 eq, 74.8 mmol) in water (100 ml) and tetrabutylammonium bisulfate (8.32 g, 0.95 eq, 24.5 mmol). The reaction mixture was stirred vigorously for 1 hour. The reaction progress was checked by TLC (Hanessians Stain) in 75% hexanes:ethyl acetate. The reaction mixture was extracted with DCM (2×50 ml). The organic layer was dried over anhydrous MgSO₄ and concentrated in vacuo. The material was then purified by silica gel flash chromatography (3:1 hexanes:ethyl acetate). ¹H NMR of the isolated material was consistent with the published structure. M+H=373.0

Preparation of 1-amino 2-acetamido 1,2-dideoxy 3,4,6-tri-O-acetyl-β-D-galactopyranose (NAG24). To NAG23 (0.26 g, 1 eq, 0.7 mmol) dissolved in ethyl acetate (25 mL) was added palladium on carbon (˜26 mg). Next a hydrogen balloon and vacuum line were inserted. The reaction mixture was evacuated 3× and purged with hydrogen after each evacuation. The reaction mixture was stirred at room temperature for 1 hour. LC/MS after 1 hour confirmed the formation of the product. The reaction mixture was filtered over a bed of Celite®, which was washed with 3×10 mL of EtOAc. The combined filtrate was concentrated in vacuo and used in the next step without further purification. M+H=346.6

Preparation of 1-amino (15′-azido-tetraethyleneglycol propanoyl) 2-acetamido 1,2-dideoxy-β-D-galactopyranoside (NAG26). To NAG24 (0.24 g, 1 eq, 0.7 mmol) dissolved in ethyl acetate (45 mL) and DIEA (0.24 mL, 2 eq, 1.4 mmol) was added azido-PEG₄-NHS (0.41 g, 1.5 eq, 1.05 mmol) in ethyl acetate (5 mL) dropwise with stirring under argon atmosphere. The reaction was allowed to stir at room temperature overnight. Completion of the reaction was verified by LC/MS. M+H=619.5. Ethyl acetate was removed in vacuo, and the resulting material was used in the next step without further purification. To NAG 25 (0.43 g, 1 eq, 0.7 mmol) dissolved in MeOH (10 mL) was added 100 μL of 25% sodium methoxide solution in methanol. The reaction mixture was stirred at room temperature for 1 hour under argon atmosphere. LC/MS after 1 hour showed only starting material, at which time were added 500 μL of a 25% sodium methoxide solution in methanol. LC/MS after 1 hour showed formation of product and disappearance of starting material. Dowex resin was added until pH of solution reached ˜7. The resin was removed by filtration, solvent was removed in vacuo, and the residue was purified by reverse phase HPLC. M+H=493.7.

### Synthesis of Monovalent GaINAc HyNic

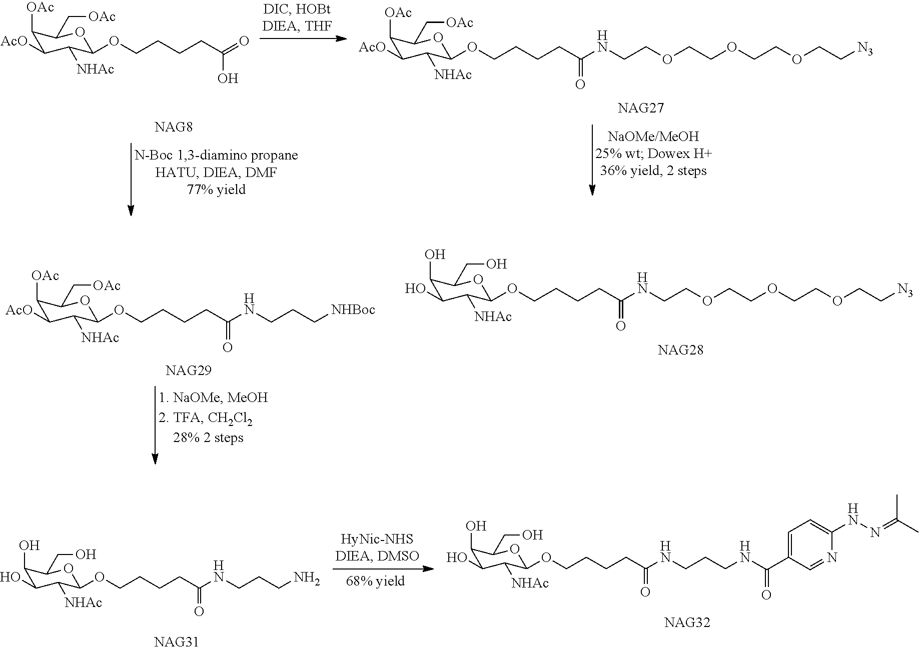 [SMILES: CC(=O)NC1C(OCCCCC(=O)O)OC(COC(C)=O)C(OC(C)=O)C1OC(C)=O ; CC(=O)NC1C(OCCCCC(=O)NCCOCCOCCOCCN=[N+]=[N-])OC(COC(C)=O)C(OC(C)=O)C1OC(C)=O ; CC(=O)NC1C(OCCCCC(=O)NCCCNC(=O)OC(C)(C)C)OC(COC(C)=O)C(OC(C)=O)C1OC(C)=O ; CC(=O)NC1C(OCCCCC(=O)NCCOCCOCCOCCN=[N+]=[N-])OC(CO)C(O)C1O ; CC(=O)NC1C(OCCCCC(=O)NCCCN)OC(CO)C(O)C1O ; CC(=O)NC1C(OCCCCC(=O)NCCCNC(=O)c2ccc(NN=C(C)C)nc2)OC(CO)C(O)C1O]

Preparation of [5-(2-{2-[2-(2-azidoethoxy)ethoxy]ethoxy}ethylamino)-5-oxopentanoyl] 2-deoxy 2-N-acetyl -3,4,6-tri-O-acetyl-β-D-galactopyranoside (NAG27). To a solution of NAG8 (1.00 g, 2.24 mmol) in THE (8 mL) was added DIC (0.56 g, 4.48 mmol) and HOBt (0.25 g, 2.17 mmol). After 1 h, a white precipitate formed, and the reaction mixture was cooled to 0° C. A solution of azido-Peg3-amine (0.63 g, 2.91 mmol) in THE (2 mL) was added, and the reaction was stirred for an additional 1 h. RP-HPLCMS showed formation of NAG27. ESI MS+ mass calcd C₂₇H₄₅N₅O₁₃: 647.7, Found: 647.8 [M+H]. The precipitate was removed by filtration, and the reaction mixture was concentrated in vacuo to give thick syrup.

Preparation of [5-(2-{2-[2-(2-azidoethoxy)ethoxy]ethoxy}ethylamino)-5-oxopentanoyl] 2-deoxy 2-N-acetyl-β-D-galactopyranoside (NAG28). Crude NAG27 was dissolved in anhydrous methanol (10 mL) and treated with NaOMe in MeOH (25 wt %, 250 μL). The reaction mixture was stirred overnight at room temperature. RP-HPLCMS showed consumption of NAG27 and formation of the NAG28. ESI MS+ mass calcd C₂₁H₃₁N₅O₁₀: 521.6, Found: 522.3 [M+H]. Dowex H+ resin was added to neutralize the base, the resin was then removed by filtration, and the liquor was concentrated in vacuo. Crude NAG28 was purified by RP-HPLC to afford 0.42 g, 36% yield over two steps.

Preparation of ([3-(tert-butoxycarbonylamino)propylamino])-5-oxopentanoyl] 2-deoxy 2-N-acetyl -3,4,6-tri-O-acetyl-β-D-galactopyranoside (NAG29). NAG8 (0.29 g, 0.65 mmol) in DMF (3 mL) was activated with HATU (0.25 g, 0.65 mmol) and DIEA (0.34 mL, 1.95 mmol). After 10 min, mono-Boc protected 1,3-diaminopropane (0.13 g, 0.72 mmol) was added, and the resulting mixture was stirred for 2 h. The mixture was concentrated in vacuo and purified by SiO₂ chromatography to provide NAG29 (0.30 g, 77% yield). ESI MS+ mass calculated C₂₇H₄₅N₃O₁₂: 603.7, Found: 626.8 [M+Na].

Preparation of ([3-(amino)propyamino])-5-oxopentanoyl] 2-deoxy 2-N-acetyl-β-D-galactopyranoside (NAG31). A solution of NAG29 (0.30 g, 0.50 mmol) in anhydrous methanol was treated with NaOMe in MeOH (25 wt %, 50 μL). After 20 min, TLC showed complete consumption of NAG29. Dowex strong H+ resin was added to acidify the reaction mixture, which was then stirred for 30 min. The resin was removed by filtration and washed with 1% TEA in MeOH and 1M NaOH (aq). The filtrate was neutralized with 1M HCl (aq) and concentrated in vacuo to give NAG31 (0.052 g, 28% yield). ESI MS+ mass calculated C₁₆H₃₁N₃O₇: 377.4, Found: 377.6 [M+H].

Preparation of ({3-[6-(isopropylidenehydrazino)-nicotinoylamino]propylamino}-5-oxopentanoyl) 2-deoxy 2-N-acetyl-β-D-gaactopyranoside (NAG32). A solution NAG31 (0.009 g, 22 μmol) in DMSO (1 mL) was treated with HyNic-sulfo-NHS (0.007 g, 18 μmol) and DIEA (9.4 μL, 54 μmol) for 1 h and purified by RP-HPLC to afford NAG32 TFA salt (0.010 g, 68% yield). ESI MS+ mass calculated C₂₅H₄₀N₆O₈: 552.6, Found: 554.0 [M+H].

### Synthesis of Glucitol Auxiliary Moiety:

### Synthesis of Di-Glucitol Azide Auxiliary Moiety

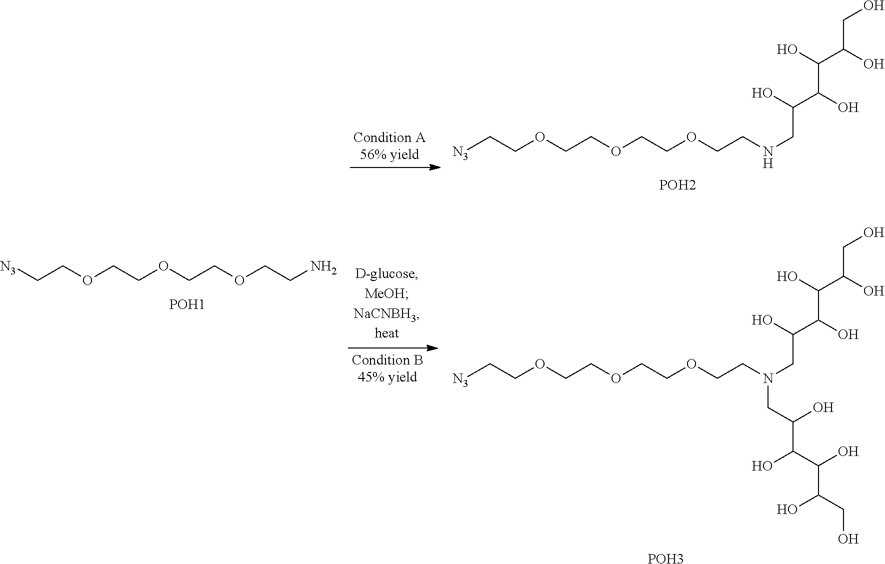

Preparation of 2-{2-[2-(2-Azidoethoxy)ethoxy]ethoxy}ethyamino D-glucitol (POH2). The reaction solution of D-glucose (0.093 g, 0.52 mmol) and amino-Peg3-azide (0.11 g, 0.52 mmol) in methanol (2 mL) was stirred at room temperature for 3 h. NaBH₃CN (0.033 g, 0.52 mmol) in 1 mL of methanol was added to the reaction mixture followed by one drop of acetic acid. The reaction mixture was stirred for 16 hours at room temperature, at which time the mixture was concentrated in vacuo and purified by preparatory HPLC to furnish 0.11 g of product POH2 as an oil (56% yield). ESI MS for C₁₄H₃₀N₄O₈ calculated 382.4, observed [M+H]⁺ 383.0.

Preparation of 2-{2-[2-(2-Azidoethoxy)ethoxy]ethoxy}ethyamino 1,1-bis(D-glucitol) (POH3). The reaction solution of D-Glucose (0.19 g, 1.04 mmol) and amino-Peg3-azide (0.11 g, 0.52 mmol) in methanol (3 mL) was stirred at room temperature for 3 h. NaBH₃CN (0.065 g, 1.04 mmol) in 1 mL of methanol was added to the reaction mixture followed by one drop of acetic acid. The reaction mixture was stirred for 16 h at room temperature, at which time the mixture was concentrated in vacuo and purified by preparatory HPLC to afford 0.13 g of product POH3 as an oil (45% yield). ESI MS for C₂₀H₄₂N₄O₁₃ calculated 546.6, observed [M+H]⁺ 547.0.

### Synthesis of Di-Glucitol HyNic Auxiliary Moiety

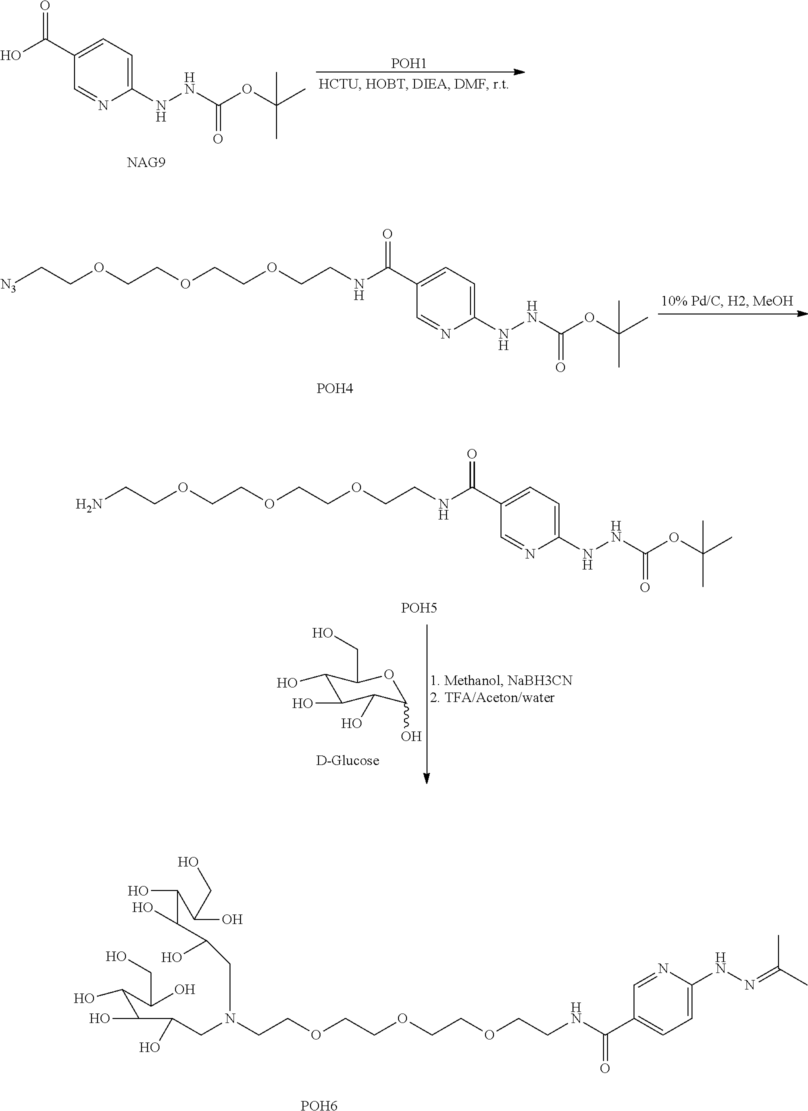

Preparation of (2-{2-[2-(2-Azidoethoxy)ethoxy]ethoxy}ethylamino){6-[(tert-butyl)-2-carboxyhydrazino]-3-pyridyl}formaldehyde (POH4). To the solution of 6-Boc-hydrazinonicotinic acid (NAG9, 0.25 g, 1.0 mmol), amino-Peg3-azide (POH1, 0.22 g, 1.0 mmol), HCTU (0.83 g, 2.0 mmol) and HOBT.H₂O (0.31 g, 2.0 mmol) in DMF (5 mL) was added DIPEA (0.70 ml, 2.0 mmol) at room temperature. The reaction mixture was stirred for 16 h, at which time the mixture was concentrated in vacuo. The crude mixture was diluted with 30 mL of dichloromethane, washed sequentially by saturated NaHCO₃ solution (10 mL) and brine (10 mL), and dried over anhydrous Na₂SO₄. The solvent was evaporated in vacuo, and the crude mixture was purified by silica gel column chromatography using (ethyl acetate, 5% methanol)/hexane solvent system (0-100% gradient on Combi Flash Rf Instrument) to give 0.098 g of intermediate POH4 as a colorless oil (22% yield). ESI MS for C₁₉H₃₇N₇O₆ calculated 453.5, observed [M+H]⁺ 454.0.

Preparation of (2-{2-[2-(2-Aminoethoxy)ethoxy]ethoxy}ethylamino){6-[(tert-butyl)-2-carboxyhydrazino]-3-pyridyl}formaldehyde (POH5). Compound POH4 (0.098 g, 0.22 mmol) and catalytic amount of Pd/Carbon (10% w/w) in 4 mL of methanol were exposed to hydrogen atmosphere at room temperature for 1 h. The reaction mixture was filtered, and the filtrate was concentrated in vacuo to yield 0.090 g of POH5 as an oil (98% yield), which was used in the next reaction without further purification. ESI MS for C₁₉H₃₃N₅O₆ calculated 427.5, observed [M+H]⁺ 428.0.

Preparation of {2-[2-(2-{2-[Bis(2,3,4,5,6-pentahydroxyhexyl)amino]ethoxy}ethoxy)ethoxy]ethylamino}[6-(isopropylidenehydrazino)-3-pyridyl]formaldehyde (POH6). The solution of D-Glucose (0.16 g, 0.86 mmol) and compound POH5 (0.09 g, 0.22 mmol) in 5 mL of methanol was stirred at room temperature for 3 h. NaBH₃CN (0.054 g, 0.86 mmol) in 1 mL of methanol was added to the reaction mixture followed by a drop of acetic acid, and the reaction mixture was stirred for 16 h at room temperature. To this suspension, another portion of D-Glucose (0.16 g, 0.86 mmol) was added, and the mixture was stirred for 3 h, at which time NaBH₃CN (0.054 g, 0.86 mmol) in 1 mL of methanol was added followed by a drop of acetic acid. The reaction mixture was further stirred for 16 h at room temperature. After purification by preparatory HPLC, the resulting product was treated with trifluoroacetic acid/acetone (90:10, v/v) for 15 min before, at which time the product was purified by preparatory HPLC. Lyophilization of the HPLC fractions gave 3.0 mg of product POH6 as oil (2% yield). ESI MS for C₂₉H₅₃N₅O₁₄ calculated 695.7, observed [M+H]⁺ 696.0.

### Synthesis of Tetra-Glucitol Azide Auxiliary Moiety

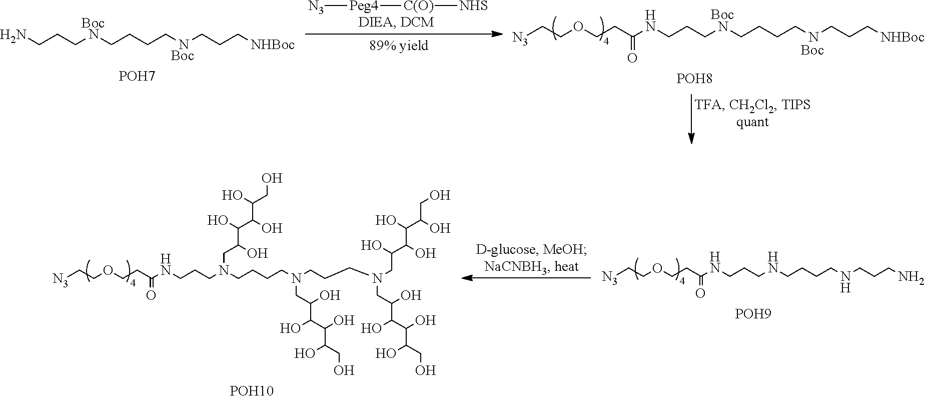

Preparation of 3-{N-tert-Butoxycarbonyl[4-({3-[-(2-{2-[2-(2-azidoethoxy)ethoxy]ethoxy}ethoxy)propionylamino]propyl}-N-tert-butoxycarbonylamino)butyl]amino}propylamino 2,2-dimethylpropionate (POH8). To a solution of POH7 (0.18 g, 0.36 mmol) in DCM (3 mL) was treated with NHS azido-PEG₄ carboxylate (0.13 g, 0.33 mmol) and DIEA (0.12 mL, 0.66 mmol). The mixture was stirred for 1 h at room temperature, and the product formation was confirmed by RP-LCMS. The reaction was concentrated in vacuo and purified by SiO₂ chromatography to afford POH8 (0.23 g, 89% yield). ESI MS+ mass calculated C₃₆H₆₉N₇O₁₁: 775.5, Found: 776.0 [M+H].

Preparation of 1-{3-[4-(3-Aminopropylamino)butylamino]propylamino}-3-(2-{2-[2-(2-azidoethoxy)ethoxy]ethoxy}ethoxy)-1-propanone (POH9). Tri-Boc POH8 (0.23 g, 0.29 mmol) in CH₂Cl₂ (2 mL) was treated with TFA (10 mL) and TIPS (0.10 mL) for 1 h to afford POH9 in quantitative yield upon concentration in vacuo. POH9 was used in the next step without further purification. ESI MS+ mass calculated C₂₁H₄₅N₇O₅: 475.4, Found: 476.0 [M+H].

Preparation of 1-{3-[4-(3-Aminopropylamino)butylamino]propylamino}-3-(2-{2-[2-(2-azidoethoxy)ethoxy]ethoxy}ethoxy)-1-propanone (POH10). The mixture of tri-amine POH9 (0.29 mmol), D-glucose (1.46 g, 8.11 mmol), and NaCNBH₃ (0.15 g, 2.34 mmol) in MeOH (10 mL) was heated to 50° C. for 4 h. A mixture of 3 and 4 additions of D-glucose were observed. The desired adduct POH10 (0.016 g) was isolated using RP-HPLC. ESI MS+ mass calculated C₄₅H₉₃N₇O₂₅: 1132.3, Found: 1132.6 [M+H].

### Synthesis of Folate Ligand:

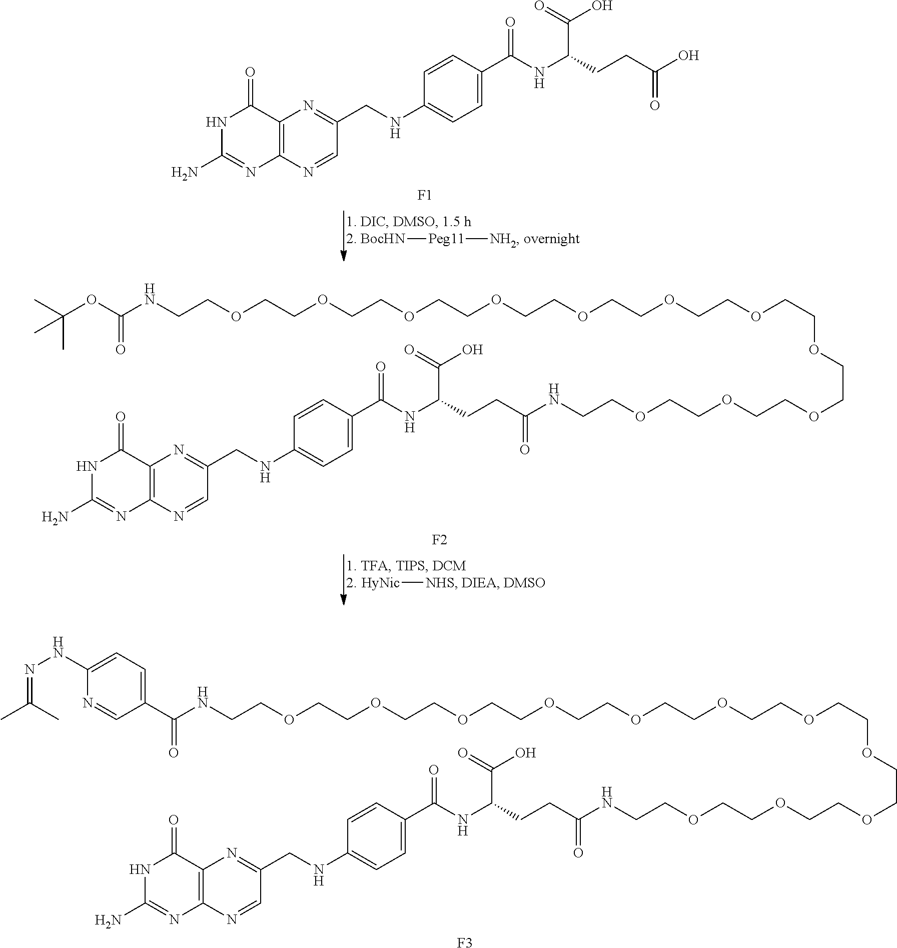

Preparation of N-Boc-Peg₁₁ folate (F2). To a solution of folic acid (225 mg, 0.51 mmol) in DMSO (4 mL) was added diisopropylcarbodiimide (80 μL, 0.51 mmol). After stirring for 1.5 h, a solution of Boc-Peg₁₁-diamine (220 mg, 0.34 mmol) in DMSO (1 mL) was added, and the reaction stirred overnight. Upon addition of water (35 mL), a precipitate formed, which was collected by filtration and purified by RP-HPLC to afford F2 (364 mg, 67% yield). MALDI-TOF mass calcd C₄₈H₇₇N₉O₁₈: 1067.54, Found: 1069.89 [M+H].

Preparation of folate-peg₁₁-HyNic acetone hydrazone (F3). MonoBoc F2 (210 mg, 0.2 mmol) was treated with TFA (9 mL) and acetone (1 mL) for 1.5 h, the resulting mixture was concentrated in vacuo, and the residue was dried under a high vacuum. MALDI-TOF mass calcd C₄₃H₆₉N₉O₁₆: 967.48, Found: 969.86 [M+H]. The crude yellowish solid was dissolved in DMSO (200 μL) and treated with a solution of HyNic-NHS ester (10.0 mg, 0.03 mmol) and DIEA (40 μL, 0.23 mmol) for 1.5 h. The crude was purified by RP-HPLC to afford F3 (1.2 mg, 3.5% yield). MALDI-TOF mass calcd C₅₂H₇₈N₁₂O₁₇: 1142.56, Found: 1144.03 [M+H].

### Synthesis of Monovalent Folate Azide

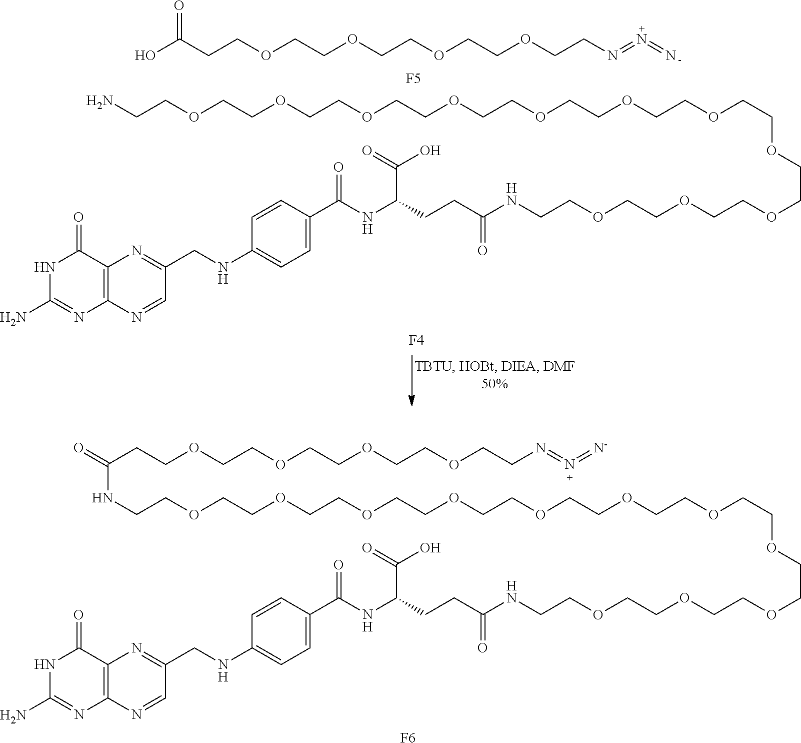

Preparation of azido-Peg₄-amido-Peg₁₁ folate (F6). Amino-Peg₁₁ folate F4 (115 mg, 0.12 mmol) in DMSO (1.0 mL) was added to a solution of azido-Peg₄ acid (38 mg, 0.13 mmol) activated with TBTU (42 mg, 0.13 mmol), HOBt (20 mg, 0.13 mmol), and DIEA (63 μL, 0.36 mmol) in DMSO (1.0 mL). After 2 h, base was removed in vacuo, and the crude was purified by RP-HPLC to afford F6 (75 mg, 50%). AP-ESI+ Mass calcd C₅₄H₈₈N₁₂O₂₁: 1240.61, Found: 1241.7 [M+H]⁺, 621.5 [M+2H]²⁺

### Synthesis of PSMA Ligands

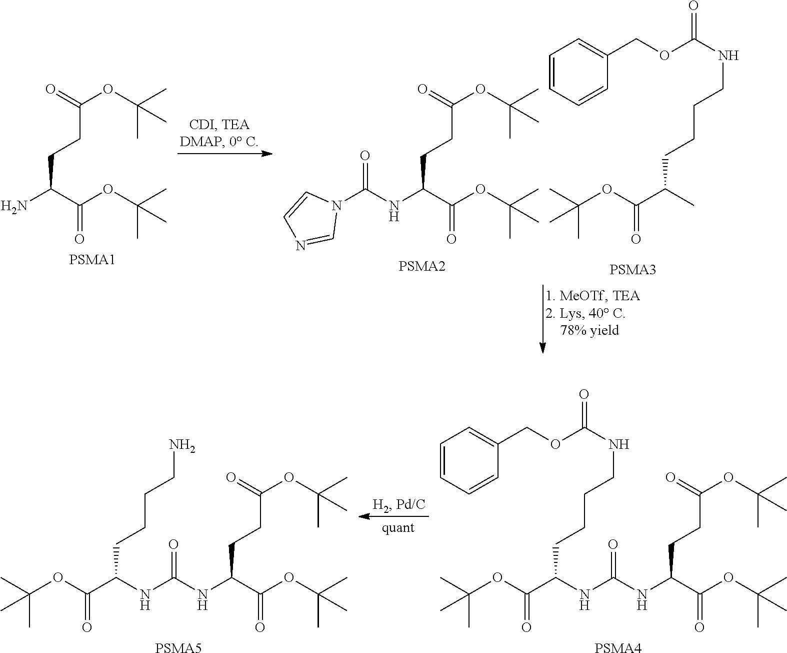

Preparation of Cbz-Lys ureido Glu tris-t-butyl ester (PSMA4). To an ice cold solution of glutamic di-tert-butyl ester (1.06 g, 3.58 mmol), DMAP (27 mg), and TEA (1.25 mL, 8.95 mmol) in CH₂Cl₂ (10.0 mL) was added CDI (638 mg, 3.94 mmol) in one portion. After 30 min, the reaction was removed from the ice bath and stirred overnight. The reaction was diluted with CH₂Cl₂ and washed with sat. NaHCO₃ (aq.), water, and brine. After drying over Na₂SO₄, the organic layer was concentrated in vacuo and dried under high vacuum to give PSMA2. A solution of PSMA2 in DCE (10 mL) was cooled to 0° C. and treated sequentially with MeOTf (0.59 g, 3.58 mmol) and TEA (1.00 mL, 7.16 mmol). After 45 min, Cbz-Lys t-butyl ester PSMA3 (1.34 g, 3.58 mmol) in DCE (2 mL) was added, and the mixture was heated to 40° C. After 2 h, the reaction was diluted with CH₂Cl₂ and washed with sat. NaHCO₃ (aq.), water, and brine. The organic layer was dried over Na₂SO₄ and concentrated in vacuo to thick syrup. The crude material was purified through SiO₂ gel chromatography to afford PSMA4 (1.73 g, 78%) as a white foam. AP-ESI+ Mass calcd C₃₂H₅₁N₃O₉: 621.36, Found: 622.4 [M+H]⁺, 644.4 [M+Na]⁺

Preparation of Lys ureido Glu tris-t-butyl ester (PSMA5). A solution of PSMA4 (1.73 g, 2.79 mmol) in EtOAc (100 mL) was degassed by application of vacuum and backfilling with argon. Palladium (10% wt on activated carbon, 0.15 g) was added in one portion, the mixture was degassed by application of vacuum and purging with H₂ (g), and stirred for 6 h. The catalyst was removed by filtration, and the mother liquor concentrated in vacuo to give PSMA5 quantitatively. AP-ESI+ Mass calcd C₂₄H₄₅N₃O₇: 487.32, Found: 488.4 [M+H]⁺

### Synthesis of Monovalent PSMA Azide (PSMA7)

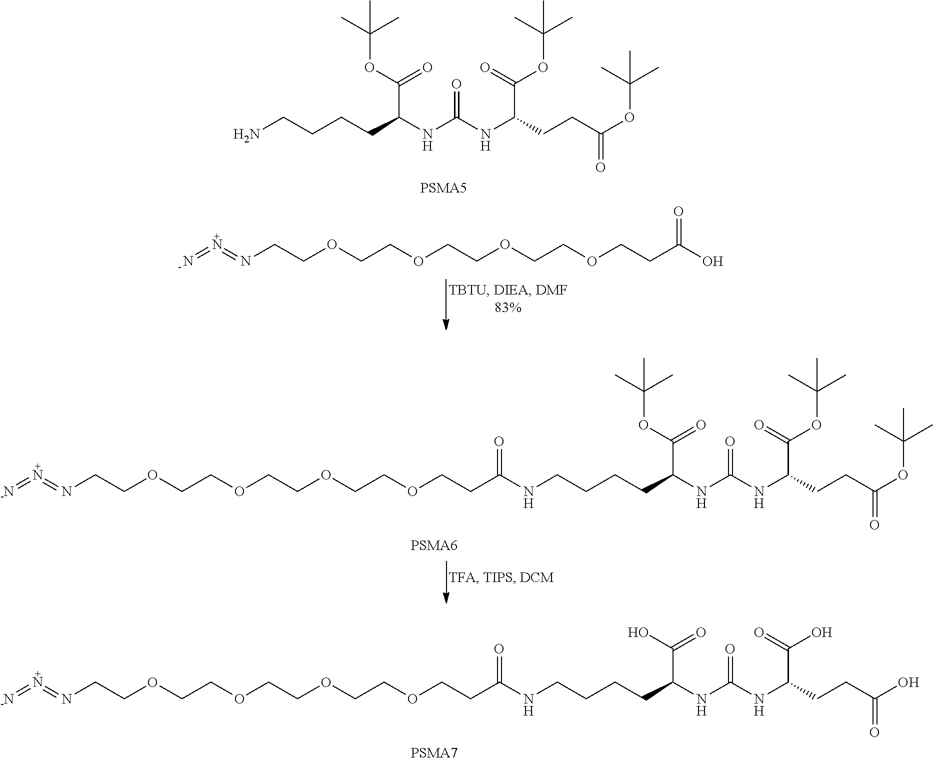

Preparation of azido-Peg₄-Lys ureido Glu tris-t-butyl ester (PSMA6). Azido-Peg₄ acid (133 mg, 0.45 mmol) was activated with TBTU (146 mg, 0.45 mmol), HOBt (69 mg, 0.45 mmol), and DIEA (216 μL, 1.24 mmol) in DMF (3.0 mL). After 15 min, a solution of PSMA5 (202 mg, 0.41 mmol) was delivered and the reaction stirred at RT for 1.5 h. RP-HPLCMS showed formation of desired product. The reaction mixture was concentrated in vacuo and purified through SiO₂ gel chromatography to afford PSMA6 (257 mg, 83%). AP-ESI+ Mass calcd C₃₅H₆₄N₆O₁₂: 760.46, Found: 761.5 [M+H]⁺, 783.5 [M+Na]⁺

Preparation of azido-Peg₄-Lys ureido Glu (PSMA7). Tris-tert-butyl ester PSMA6 (257 mg, 0.34 mmol) was treated with a solution of TFA:TIPS (10 mL, 97.5:2.5, v/v) for 30 min. RP-HPLCMS showed complete conversion to the desired product. The reaction mixture was concentrated in vacuo and purified by RP-HPLC to afford PSMA7 (112 mg, 56%). AP-ESI+ Mass calcd C₂₃H₄₀N₆O₁₂: 592.27, Found: 593.3 [M+H]⁺

### Synthesis of Monovalent PSMA HyNic (PSMA10)

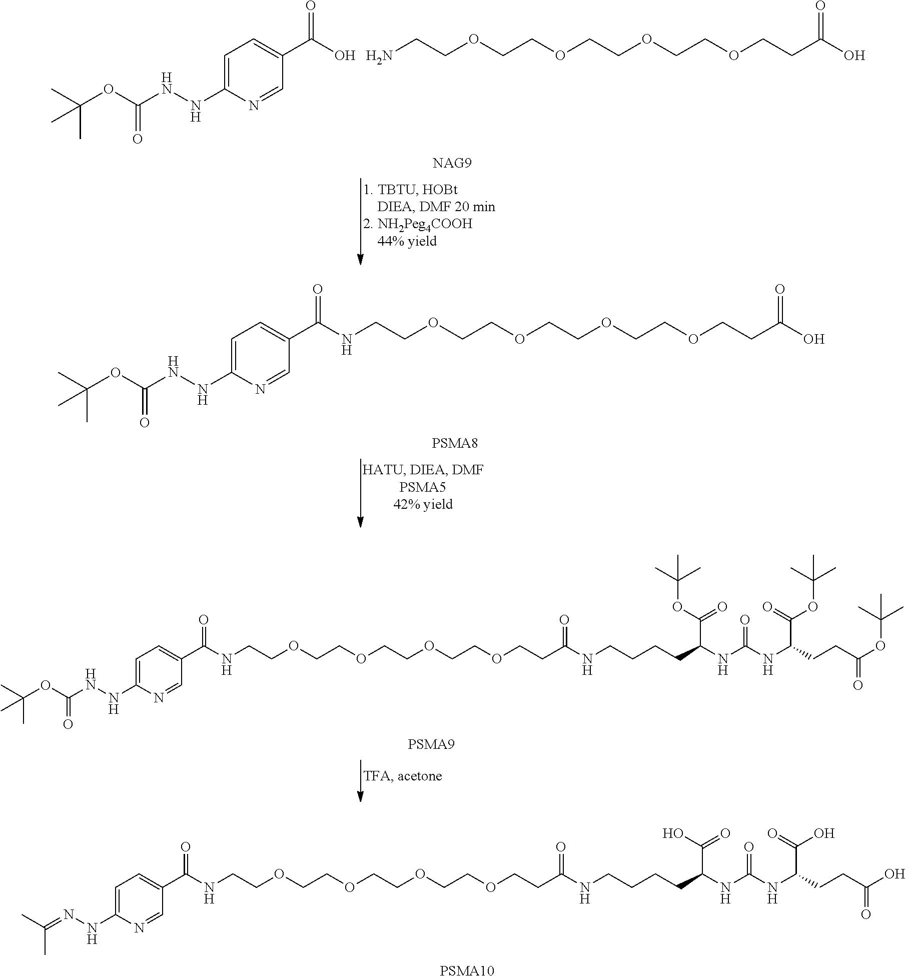

Preparation of N-Boc 4-hydrazino-nicotinamido Peg₄ acid (PSMA8). N-Boc 4-hydrazino nicotinic acid NAG9 (137 mg, 0.54 mmol) was treated with TBTU (124 mg, 0.49 mmol), HOBt (83 mg, 0.54 mol), and DIEA (128 μL, 0.74 mmol) in DMF for 20 min. To the activated ester, was added a solution of amino-Peg₄-acid (130 mg, 0.49 mmol), and the mixture was stirred for 2 h. The reaction was concentrated in vacuo and purified through SiO₂ gel chromatography to afford PSMA8 (107 mg, 44%). AP-ESI+ Mass calcd C₂₂H₃₆N₄O₉: 500.25, Found: 501.3 [M+H]⁺

Preparation of N-Boc 4-hydrazino-nicotinamido Peg₄-epsilon-amido lys-alpha-ureido-glu tri-t-butyl ester (PSMA9). PSMA8 (107 mg, 0.21 mmol) was treated with HATU (81 mg, 0.21 mmol) and DIEA (93 μL, 0.53 mmol) in the presence of amine PSMA5 (104 mg, 0.21 mmol) in DMF for 1 h. Then the reaction mixture was concentrated in vacuo and purified through SiO₂ gel chromatography to afford PSMA9 (85 mg, 42%). AP-ESI+ Mass calcd C₄₆H₇₉N₇O₁₅: 969.46, Found: 760.6 [M+H]⁺

Preparation of dimethyl 4-hydrazono nicotinamido Peg₄-epsilon-amido lys-alpha-ureido-glu (PSMA10). Tris-t-butyl ester PSMA9 (85 mg, 0.09 mmol) was treated with a solution of TFA:acetone (10 mL, 97.5:2.5, v/v) for 30 min. RP-HPLCMS showed complete conversion to the desired product. The reaction mixture was concentrated in vacuo and purified by RP-HPLC to afford PSMA10 (55 mg, 84%). AP-ESI+ Mass calcd C₃₂H₅₁N₇O₁₃: 741.35, Found: 742.4 [M+H]⁺

### Synthesis of Bivalent PSMA Azide (PSMA18)

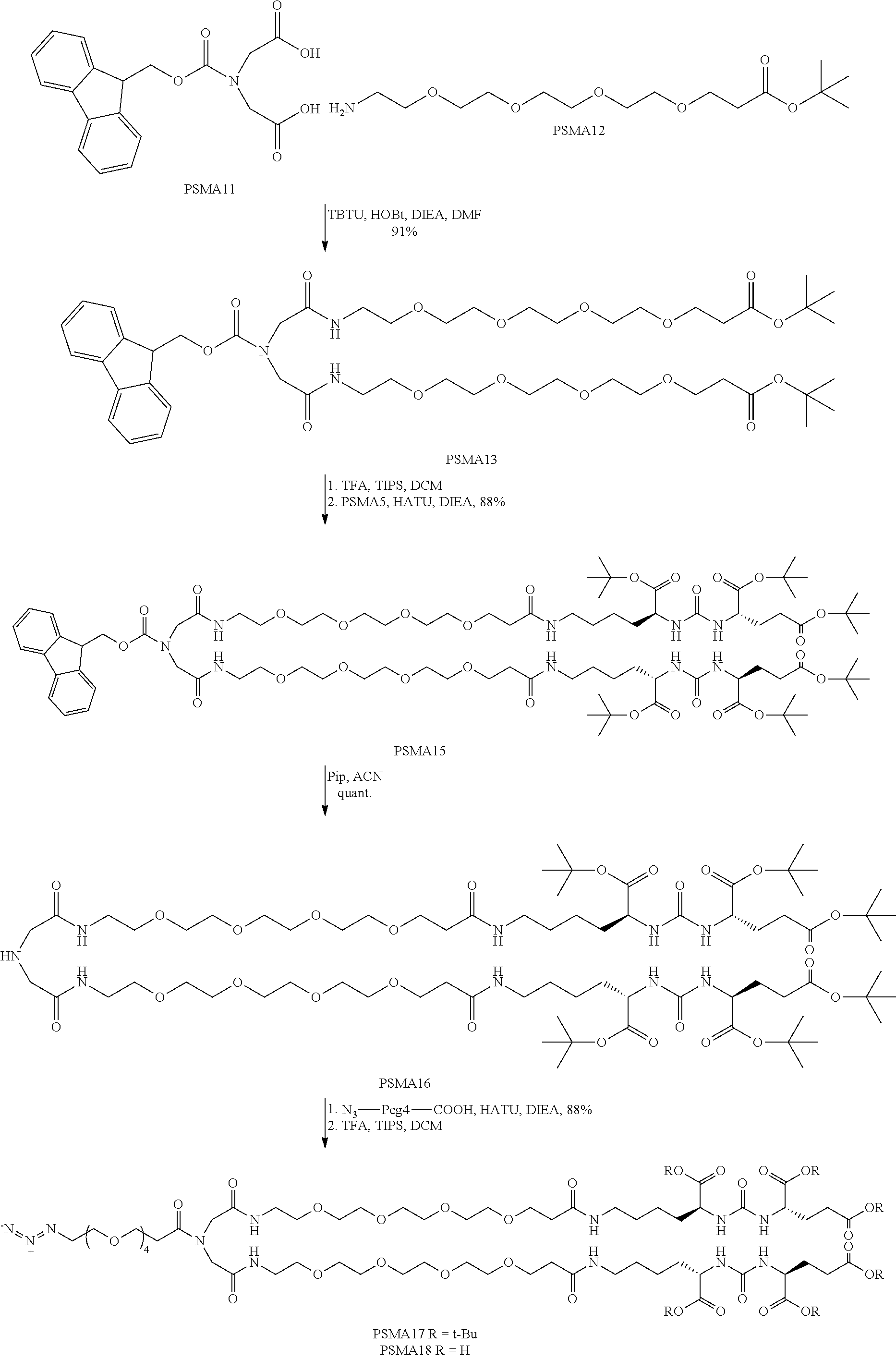

Preparation of N-Fmoc bis-imino-(acetamido-Peg₄ t-butyl ester) (PSMA13). N-Fmoc imino diacetic acid, PSMA11, (107 mg, 0.30 mmol) was treated with PSMA12 (212 mg, 0.66 mmol), TBTU (193 mg, 0.60 mmol), HOBt (92 mg, 0.60 mmol), and DIEA (209 μL, 1.20 mmol) in DMF for 2 h. The reaction was concentrated in vacuo and purified by SiO₂ gel chromatography to afford PSMA13 (250 mg, 91%). AP-ESI+ Mass calcd C₄₉H₇₅N₃O₁₆: 961.51, Found: 962.6 [M+H]⁺, 984.6 [M+Na]⁺

Preparation of N-Fmoc bis-imino-(acetamido-Peg4-epsilon-amido lys-alpha-ureido-glu tri-t-butyl ester) (PSMA15). Di-t-butyl ester PMSA13 (250 mg, 0.26 mmol) in DCM (1 mL) was treated with TFA (10 mL) and TIPS (111 μL, 0.54 mmol). After 30 min, the reaction was concentrated in vacuo to afford a syrup, which was washed with hexanes to afford di-acid PSMA14 as a thick syrup. PSMA14 was treated with HATU (198 mg, 0.54 mmol), PSMA5 (292 mg, 0.57 mmol), and DIEA (362 μL, 2.08 mmol) in DMF for 1 h. The reaction mixture was concentrated in vacuo and purified through SiO₂ gel chromatography to afford PSMA15 (408 mg, 88%). PSMA14: AP-ESI+ Mass calcd C₄₁H₅₉N₃O₁₆: 849.39, Found: 850.5 [M+H]⁺, 872.5 [M+Na]⁺. PSMA15: AP-ESI+ Mass calcd C₈₉H₁₄₅N₉O₂₈: 1788.02, Found: 895.3 [M+2H]²⁺, 917.2 [M+2Na]²⁼⁺

Preparation of bis-imino-(acetamido-Peg₄-epsilon-amido lys-alpha-ureido-glu tri-t-butyl ester) (PSMA16). N-Fmoc PMSA15 (408 mg, 0.22 mmol) in acetonitrile (10 mL) was treated with piperidine for 30 min. The reaction mixture was concentrated in vacuo, azeotroped with PhMe (3×10 mL), washed with hexanes (3×20 mL), and dried under high vacuum to afford PSMA16. AP-ESI+ Mass calcd C₇₄H₁₃₅N₉O₂₆: 1565.95, Found: 895.3 [M+2H]²⁺, 917.2 [M+2Na]²⁺

Preparation of azido-Peg₄-imido-bis-(acetamido-Peg4-epsilon-amido lys-alpha-ureido-glu tri-t-butyl ester) (PSMA17). Amine PMSA16 (172 mg, 0.11 mmol) was added to N₃-Peg₄-COOH (40 mg, 0.14 mmol) activated with HATU (52 mg, 0.14 mmol) and DIEA (116 μL, 0.66 mmol) in DMF (2 mL). After 1 h, the reaction mixture was concentrated in vacuo and purified by SiO₂ gel chromatography to afford PSMA17 (194 mg, 91%). AP-ESI+ Mass calcd C₈₅H₁₅₄N₁₂O₃₁: 1839.08, Found: 895.3 [M+2H]²⁺, 917.2 [M+2Na]²⁺

Preparation of azido-Peg₄-imido-bis-(acetamido-Peg₄-epsilon-amido lys-alpha-ureido-glu) (PSMA18). Hexa-t-butyl ester PSMA17 (194 mg, 0.10 mmol) was treated with a solution of TFA:acetone (10 mL, 97.5:2.5, v/v) for 30 min. RP-HPLCMS showed complete conversion to the desired product. The reaction mixture was concentrated in vacuo and purified by RP-HPLC to afford PSMA18 (69.4 mg, 44%). AP-ESI+ Mass calcd C₆₁H₁₀₆N₁₂O₃₁: 1502.70, Found: 752.5 [M+2H]²⁺

### Synthesis of Bivalent PSMA HyNic (PSMA20)

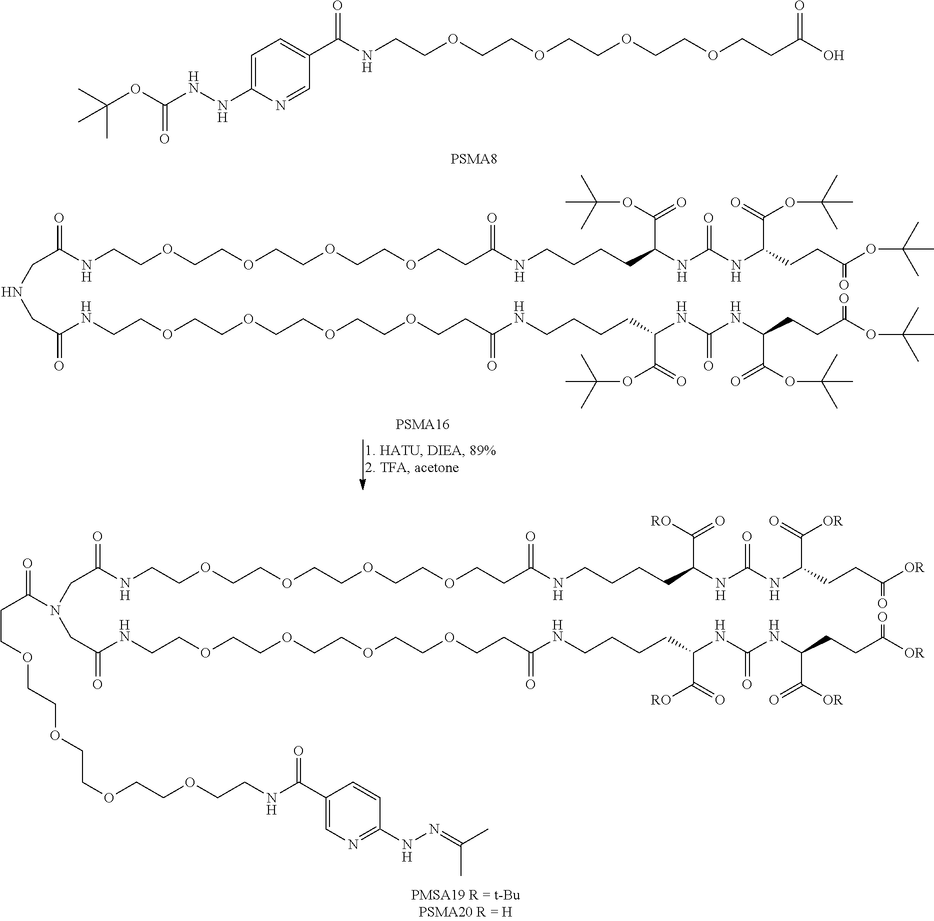

Preparation of N-Boc 4-hydrazino-nicotinamido Peg₄-imido-bis-(acetamido-Peg₄-epsilon-amido lys-alpha-ureido-glu tri-t-butyl ester) (PSMA19). Amine PMSA16 (172 mg, 0.11 mmol) was added to PSMA8 (61 mg, 0.12 mmol) activated with HATU (46 mg, 0.12 mmol) and DIEA (116 μL, 0.66 mmol) in DMF (2 mL). After 1 h, the reaction mixture was concentrated in vacuo and purified by SiO₂ gel chromatography to afford PSMA19 (201 mg, 89%). AP-ESI+ Mass calcd C₉₆H₁₆₉N₁₃O₃₄: 2048.19, Found: 1025.3 [M+2H]²⁺, 684.0 [M+3H]³⁺

Preparation of dimethyl 4-hydrazono-nicotinamido-Peg4-imido-bis-(acetamido-Peg4-epsilon-amido lys-alpha-ureido-glu) (PSMA20). Hexa-t-butyl ester PSMA19 (201 mg, 0.10 mmol) was treated with a solution of TFA:acetone (10 mL, 9:1, v/v) for 60 min. RP-HPLCMS showed complete conversion to the desired product. The reaction mixture was concentrated in vacuo and purified by RP-HPLC to afford PSMA20 (69.4 mg, 44%). AP-ESI+ Mass calcd C₇₀H₁₁₇N₁₃O₃₂: 1651.79, Found: 827.1 [M+2H]²⁺

### Synthesis of Mannose Ligand:

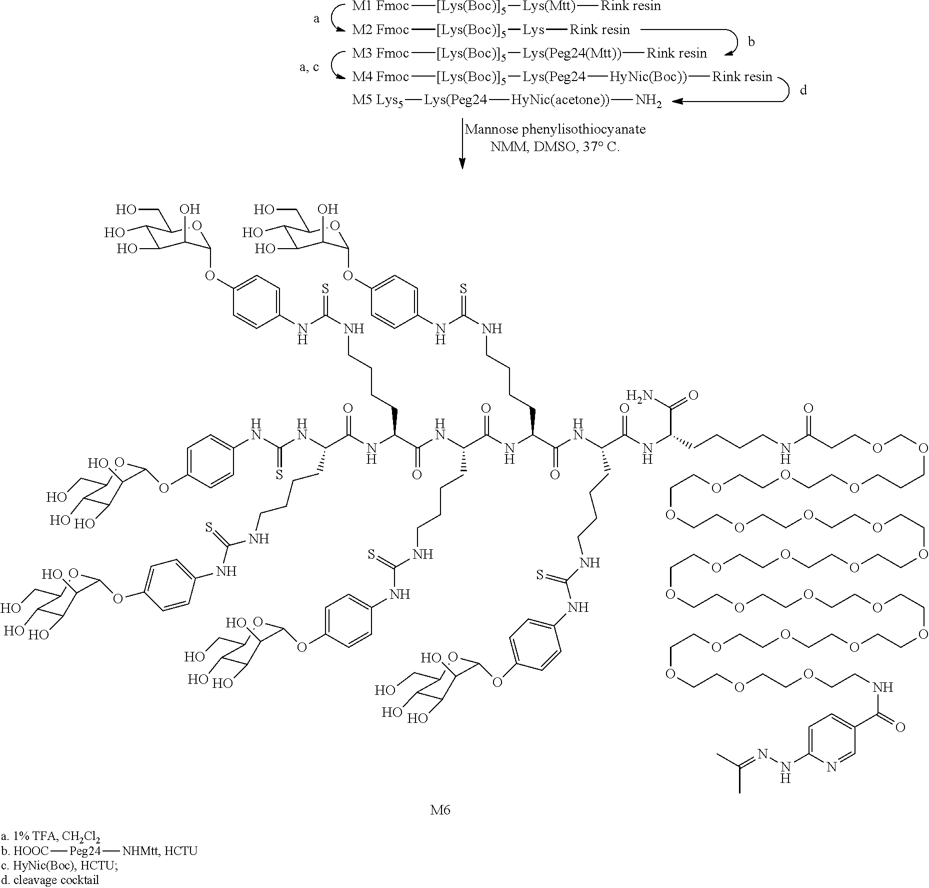

Preparation of Lys₆-Peg₂₄-HyNic (M5). Peptide scaffold was synthesized using standard Fmoc chemistry on a Rink amide resin (0.61 mmol/g) with HCTU coupling and 20% piperidine deprotection. In short, peptide M1 was prepared on an automated synthesizer on a 25 μmol scale. After deprotection of Lys(Mtt), Peg₂₄ amino(Mtt) acid was coupled to provide M3. Removal of the Mtt group and subsequent coupling of BocHyNic provided M4. Release of the peptide from the resin using trifluoroacetic acid:triisopropylsilane:water:acetone:dithithreitol (90:2:2:3:3) and purification by RP-HPLC afforded M5 (7.0 mg). AP-ESI+ Mass calcd C₉₆H₁₈₅N₁₇O₃₂: 2088.33, Found: 1046 m/2z, 698 m/3z, 524 m/4z.

Preparation of Man6-Lys6-Peg24-HyNic (M6). Peptide scaffold M5 (7.0 mg) in DMSO (1 mL) was treated with mannose isothiocyanate (8.0 mg) and N-methylmorpholine (NMM; 200 μL). The reaction was stirred for 4 h at 37° C. and purified by RP-HPLC to afford M6 (1.2 mg). MALDI-TOF mass calcd C₁₇₄H₂₇₅N₂₃O₆₈S₆: 3966.70, Found: 3987.39 [M+Na].

### Synthesis of Hexavalent Mannose Azide (M9)

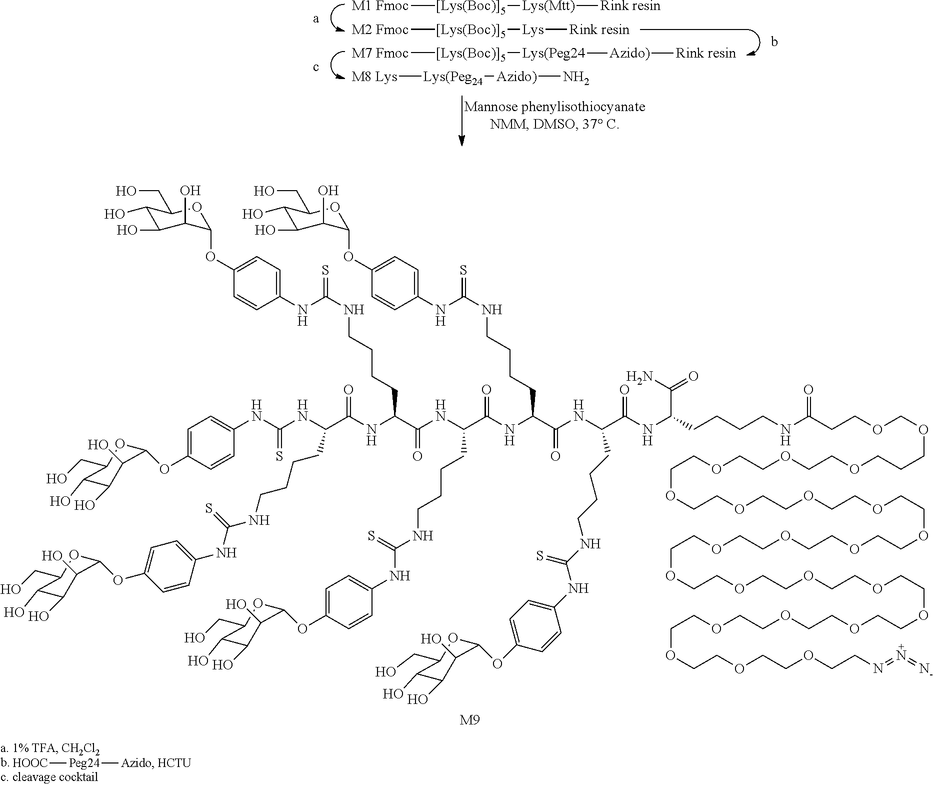

Preparation of Lys₆-Peg₂₄-Azide (M8). Peptide scaffold was synthesized using standard Fmoc chemistry on a Rink amide resin (0.61 mmol/g) with HCTU coupling and 20% piperidine deprotection. In short, peptide M1 was prepared on an automated synthesizer on a 100 μmol scale. After deprotection of Lys(Mtt), Azido-Peg₂4 acid was coupled to provide M7. Release of the peptide from the resin using the cocktail TFA:TIPS:H₂O (92.5:2.5:5) afforded M8 (167.0 mg). MALDI TOF Mass calcd C₈₇H₁₇₄N₁₆O₃₁: 1940.4, Found: 1941.1

Preparation of Man₆-Lys₆-Peg₂₄-Azide (M9). Peptide scaffold M4 (167.0 mg) in DMSO (2 mL) was treated with mannose isothiocyanate and NMM (500 μL). The reaction mixture was stirred at 37° C. and monitored by MALDI TOF until full conversion to the desired product was achieved (a total of 58 mgs of mannose isothyocyanate was added). The final product was purified by RP-HPLC to afford M9 (22 mg). MALDI-TOF mass calcd C₁₆₅H₂₆₄N₂₂O₆₇S₆: 3820.37, Found: 3843.79 [M+Na].

### Synthesis of Trivalent Mannose Azide (M15)

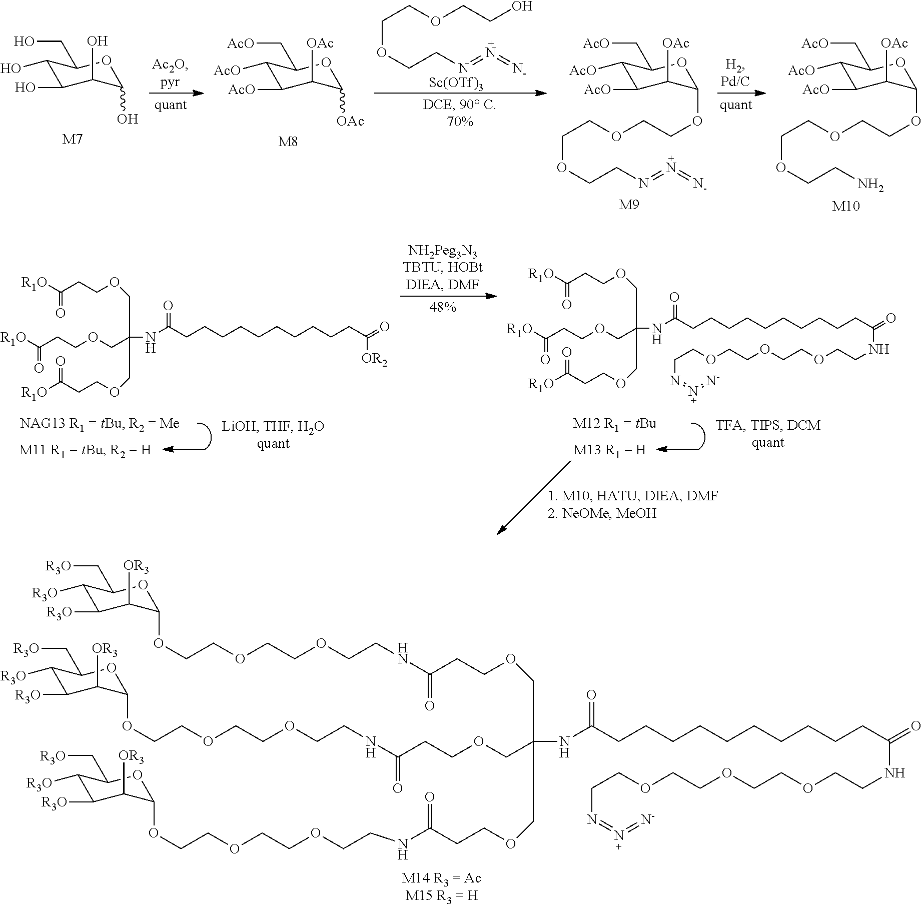

Preparation of azido tri-mannose (M15): D-Mannose was peracetylated by Ac₂O in pyridine overnight. Concentration by rotary evaporation followed by azeotroping with PhMe provided the pentaacetate (M8) in quantitative yield. Activation of M8 with Sc(OTf)₃ in the presence of commercially available azido-Peg₂ alcohol afforded azido-Peg₂ mannoside (M9), which was hydrogenated quantitatively to amine (M10). In the meanwhile, the methyl ester of tris linker (NAG13) was hydrolyzed to selectively to acid (M11). Coupling of commercially available azido-Peg₃ amine to M11 by TBTU activation provided azido tris linker (M12). Treatment of tri t-butyl ester M12 with TFA gave tri-acid M13. Coupling of M10 to M13 was mediated by HATU, and the crude mixture was globally de-acetylated to afford azido tri-mannose (M15).

### Synthesis of Monovalent Mannose Phosphoramidite (M21)

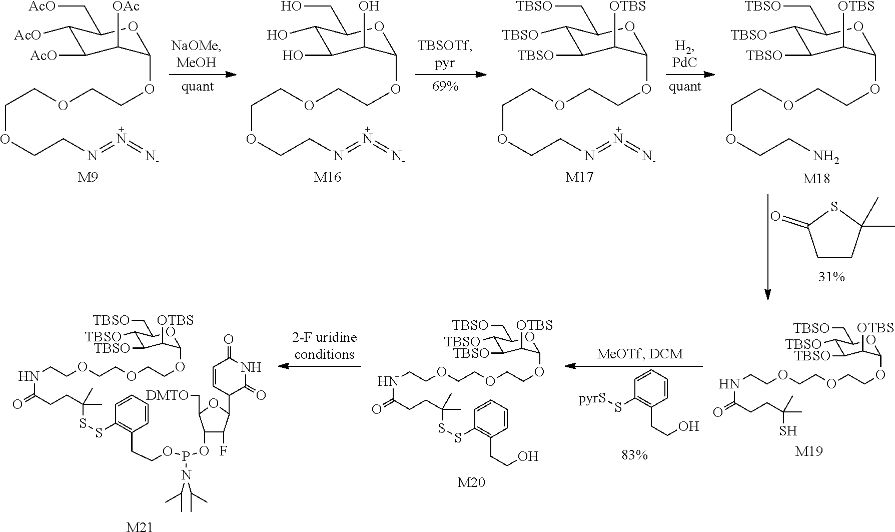

Preparation of mannose disulfide 2-fluoro uridine phosphoramidite (M21): Through standard protection/deprotection chemistry, the acetates of M9 were converted to t-butyl silyl (TBS) M17 through deacetylated intermediate M16. Reduction of azide M17 to amine M18 by hydrogenation facilitated N-acylation with the hindered thiolactone to afford thiol M19. Disulfide M20 was cleanly formed through addition of aryl mercapto-thiopyridine, pre-activated with MeOTf. Phosphoramidite M21 was to be formed in a standard 2-step one-pot manner by treatment of 2-fluoro uridine with bis(diisopropylamino) chlorophosphine followed by addition of sugar disulfide M20.

### Synthesis of Hexavalent Mannose Azide (M30)

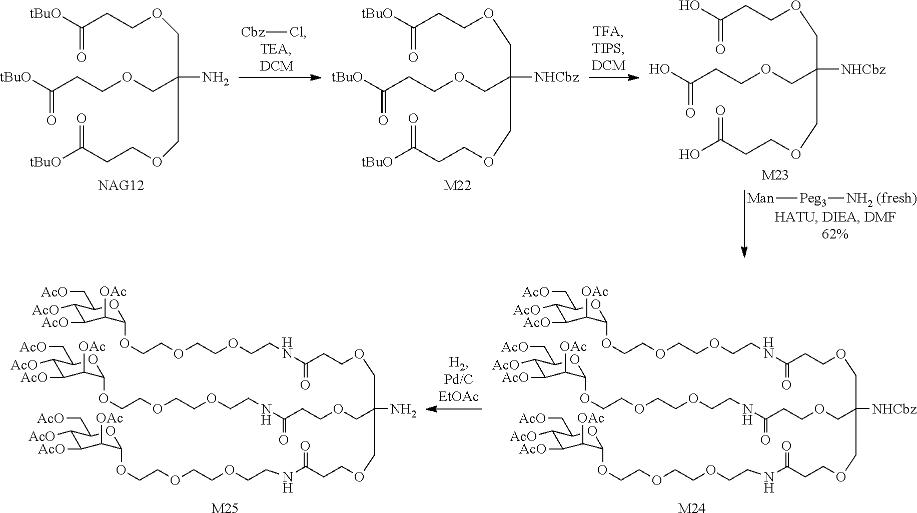

Preparation of N-carbobenzyloxy tris-(t-butoxycarboethoxymethyl)-methylamide (M22): To a solution of NAG12 (3.55 g, 7.02 mmol) in CH₂Cl₂ (12 mL) cooled in an ice bath was added Cbz-Cl (35% in PhMe, 7.3 mL) and TEA (3.9 mL). The reaction mixture was warmed to rt and stirred overnight. The mixture was diluted with CH₂Cl₂, washed with saturated NaHCO₃ (aq), dried over Na₂SO₄, and concentrated in vacuo. The crude product purified by SiO₂ chromatography to afford M22 (0.98 g, 22% yield). AP-ESI+ Mass calcd C₃₃H₅₃NO₁₁: 639.4, Found: 662.4 [M+Na]⁺

Preparation of N-carbobenzyloxy tris-((2,3,4,6-tetra-O-acetyl-1-O-α-D-mannopyranosyl)-Peg₃-amidoethoxymethyl)-methylamide (M24): Tris-t-butyl ester M22 (0.97 g, 1.51 mmol) and TIPS (0.93 mL, 4.55 mmol) in CH₂Cl₂ (5 mL) was treated with TFA (20 mL) for 5 h. The mixture was concentrated in vacuo, the oily residue was washed with hexanes and dried under high vacuum to provide M23. AP-ESI+ Mass calcd C₂₁H₂₉NO₁₁: 471.2, Found: 493.9 [M+Na]⁺

Crude M23 in DMF (5 mL) was cooled on an ice bath and treated with HATU (0.62 g, 1.63) and DIEA (0.65 mL, 3.71 mmol). After stirring for 20 min, a solution of M10 (0.89 g, 1.86 mmol) in DMF (5 mL) was added, and the mixture was warmed to rt and stirred for 3 h. The solvent was removed in vacuo, and the crude product was dissolved in EtOAc, washed with saturated NaHCO₃ (aq), dried over Na₂SO₄, and concentrated in vacuo. Purification by SiO₂ chromatography afforded M24 (0.49 g, 62% yield). MALDI-TOF Mass calcd C₈₁H₁₂₂N₄O₄₄: 1854.74, Found: 1850.14

Preparation of tris-((2,3,4,6-tetra-O-acetyl-1-O-α-D-mannopyranosyl)-Peg3-amidoethoxymethyl)-methylamine (M25): A solution of M24 (0.49 g, 0.26 mmol) was dissolved in EtOAc (50 mL) with HOAc (0.2 mL) was degassed by application of vacuum and backfilling with Ar (g). Pd on activated carbon (0.16 g) was added, and the mixture was evacuated and then purged with H₂ (g) thrice. The reaction mixture was stirred for 2 days, the catalyst was removed by filtration, and the mother liquor was concentrated in vacuo to afford M25. AP-ESI+ Mass calcd C₇₃H₁₁₆N₄O₄₂: 1720.7, Found: 1723.42

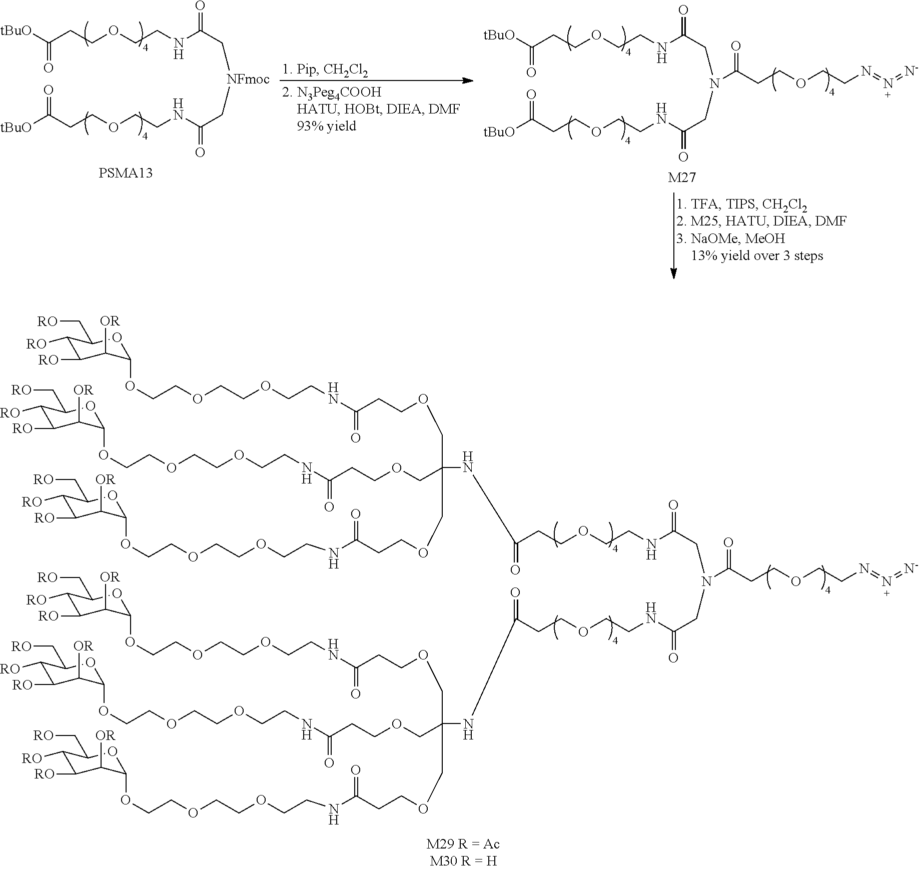

Preparation of azido-Peg₄-imido-bis-(acetamido-Peg₄-t-butyl ester) (M27): N-Fmoc PSMA13 (0.72 g, 0.75 mmol) in CH₂Cl₂ was treated with piperidine (0.75 mL) for 1 h. HPLCMS showed complete conversion to M26, AP-ESI+ Mass calcd C₃₄H₆₅N₃O₁₄: 739.4, Found: 740.5 [M+H]⁺.

The mixture was concentrated in vacuo and azeotroped with PhMe. Crude M26 was reacted with solution of azido-Peg₄ acid (0.44 g, 1.51 mmol), HATU (0.57 g, 1.51 mmol), and DIEA (0.52 mL) in DMF (5 mL) for 1 h. After solvent removal in vacuo, the crude was dissolved in EtOAc, washed with sat NaHCO₃ (aq.), dried over Na₂SO₄, and concentrated in vacuo. Purification by SiO₂ chromatography afforded M27 (0.71 g, 93% yield, 2 steps). AP-ESI+ Mass calcd C₄₅H₈₄N₆O₁₉: 1012.6, Found: 1013.6 [M+H]⁺

Preparation of azido-Peg₄-imido-bis-(trimer mannose) (M30): Imido linker M27 (0.69 g, 0.68 mmol) was treated with TIPS (0.28 mL, 1.36 mmol) and TEA (10 mL) to afford tri acid M28; AP-ESI+ Mass calcd C₃₇H₆₈N₆O₁₉: 900.5, Found: 900.9 [M+H]⁺, 922.9[M+Na]⁺. Volatiles were removed in vacuo and M28 dried under high vacuum. Di-acid M28 (82.0 mg, 0.09 mmol) was activated with HATU (75 mg, 0.2 mmol) and DIEA (0.28 mL) in DMF (2 mL) at ° C. After 30 min, a solution of M25 (0.26 mmol) in DMF (2 mL) was added, and the mixture was warmed to rt and stirred for 2 h. RP-HPLCMS showed complete conversion to M29; Mass calcd C₁₈₃H₂₉₆N₁₄O₁₀₁: 4305.84. MALDI-TOF Found: 4303.36 AP-ESI+ Found: 1436.1 [M+3H]³⁺, 1077.3 [M+4H]⁴⁺. The reaction was diluted with CH₂Cl₂ washed with sat NaHCO₃ (aq.), dried over Na₂SO₄, and concentrated in vacuo. Crude M29 (538 mg) was dissolved in MeOH (20 mL) was treated with NaOMe (25 wt % in MeOH, 0.5 mL) for 1 h. RP-HPLCMS showed complete conversion to M30. The reaction was quenched by addition of Dowex H+ resin to neutralize. The crude material was purified by HPLC to afford M30 (38.1 mg, 13% yield over 3 steps). Mass calcd C₁₃₅H₂₄₈N₁₄O₇₇: 3297.59, MALDI-TOF Found: 3318.61 [M+Na]⁺ AP-ESI+ Found: 1100.0 [M+3H]³⁺, 825.3 [M+4H]⁴⁺.

### Synthesis of ABL Ligands (ABL)

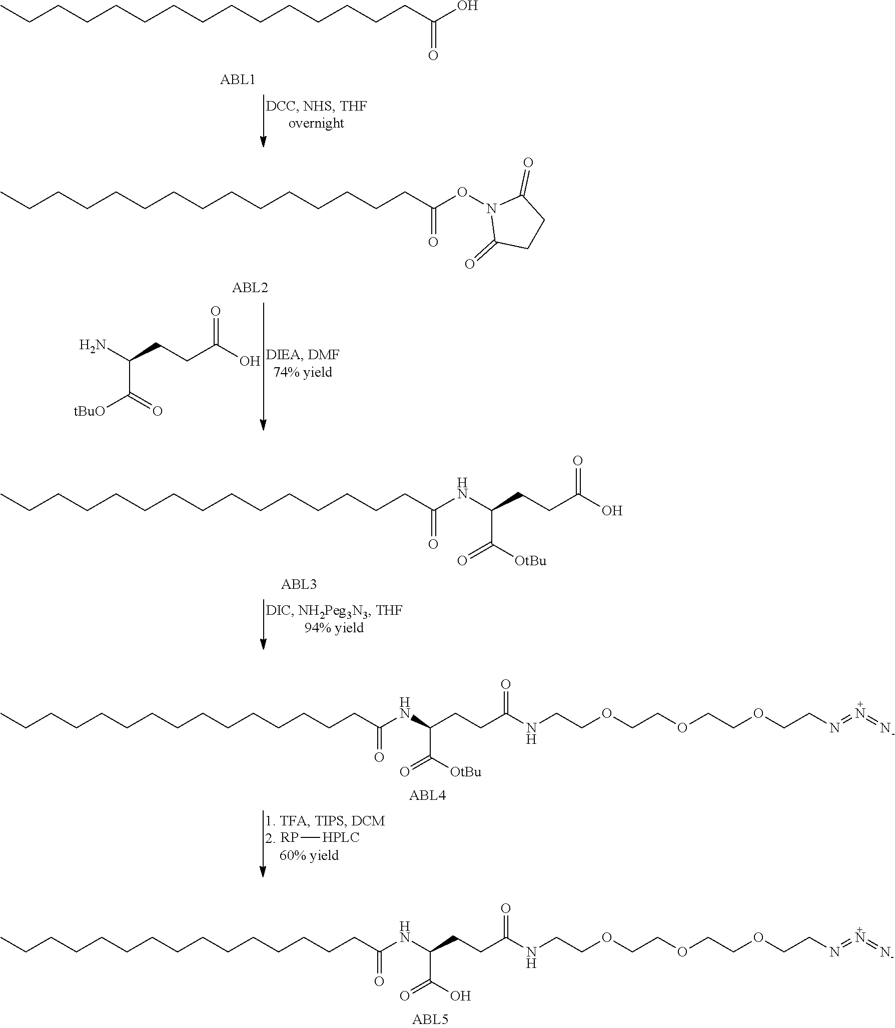

Preparation of N-palmitoyl L-glutamic acid α-t-butoxy ester (ABL3): Palmitic acid ABL1 (1.0 g, 3.8 mmol) in THE (10 mL) was treated with N-hydroxy succinimide (0.9 g, 7.6 mmol) and diisopropylcarbodiimide (1.2 mL, 7.6 mmol) overnight to afford ester (ABL2). The precipitate was removed by filtration, and the volatiles were evaporated in vacuo. The resulting residue was dissolved in DMF (6 mL) and treated with glutamic acid t-butyl ester (0.7 g, 3.4 mmol) and DIEA (1.8 mL, 10 mmol). After 2 h, the reaction mixture was diluted with water, and the desired product was extracted with Et₂O. The ether layer was dried over Na₂SO₄, concentrated in vacuo, and the crude mass was purified by SiO₂ chromatography to afford off-white solid ABL3 (1.2 g, 74% yield). AP-ESI+ Mass calcd C₂₅H₄₇NO₅: 441.3, Found: 464.0 [M+Na]⁺

Preparation of N-palmitoyl 6-(amido Peg₃ azide) L-glutamic acid α-t-butoxy ester (ABL4): To a solution of ABL3 (1.24 g, 2.8 mmol) in THE (10 mL) was added 11-azido-Peg₃ amine (0.92 g, 4.2 mmol) and diisopropylcarbodiimide (0.87 mL, 5.6 mmol). After stirring overnight, the precipitate was removed by filtration, mother liquor was concentrated in vacuo, and the crude mass purified by SiO₂ chromatography to afford an off-white solid ABL4 (1.7 g, 94% yield). AP-ESI+ Mass calcd C₃₃H₆₃N₅O₇: 641.5, Found: 642.4 [M+H]⁺

Preparation of N-palmitoyl δ-(amido Peg₃ azide) L-glutamic acid (ABL5): A solution of t-butyl ester ABL4 (1.71 g, 2.66 mmol) and TIPS (0.54 mL, 2.66 mmol) in DCM (2 mL) was treated with TFA (10 mL). After 1.5 h, the mixture was concentrated in vacuo. The oily crude was washed with hexanes, dried in vacuo, and purified by RP-HPLC to afford ABL5 (930 mg, 60% yield). AP-ESI+ Mass calcd C₂₉H₅₅N₅O₇: 585.4, Found: 586.0 [M+H]⁺

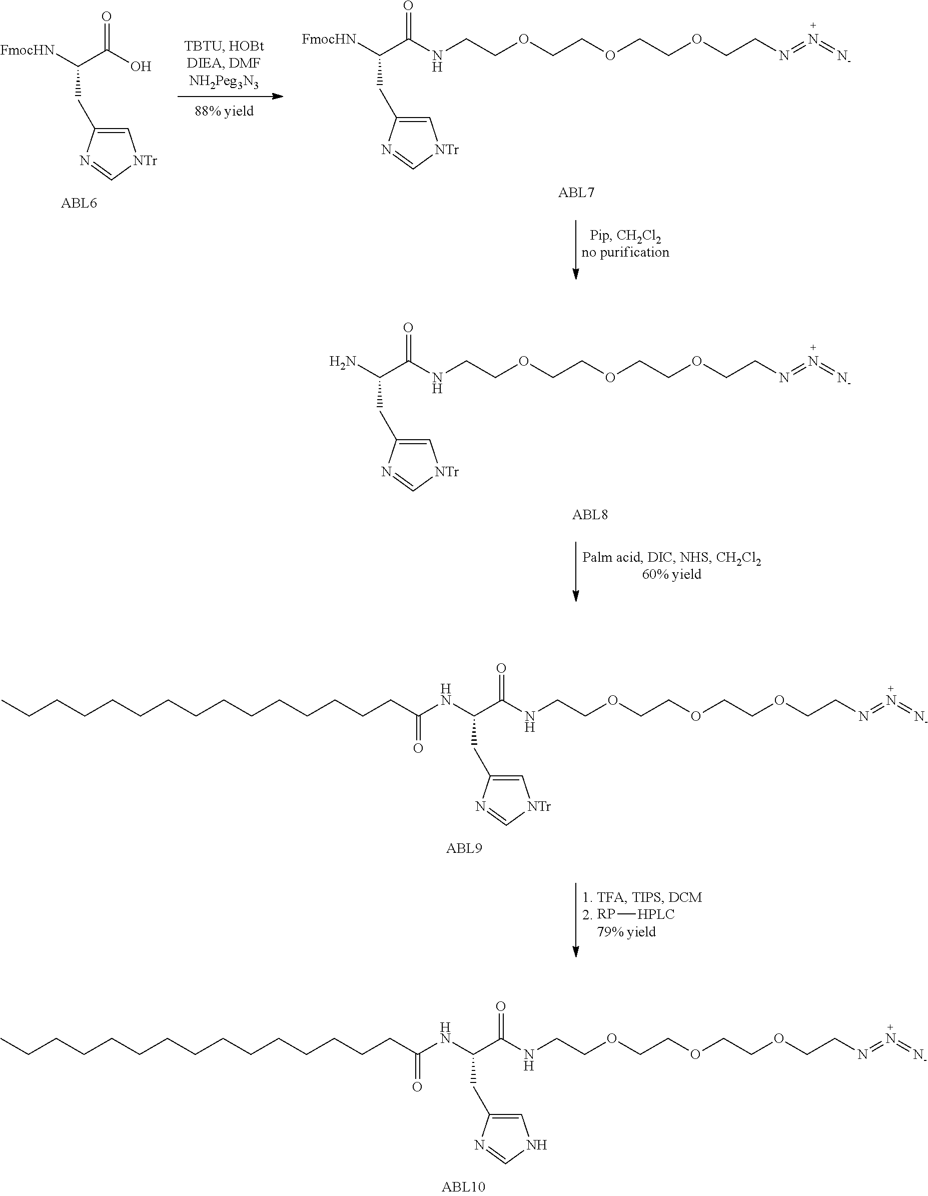

Preparation of N-α-Fmoc N-imidazyl-trityl α-(amido Peg₃ azide) L-histidine (ABL7): N-α-Fmoc N-imidazolyl-trityl L-histidine (1.00 g, 1.61 mmol) in DMF (5 mL) was activated with TBTU (0.57 g, 1.77 mmol), HOBt (0.27 g, 1.77 mmol), and DIEA (0.84 mL, 4.84 mmol) for 20 min. A solution of 11-azido-Peg₃ amine (0.35 g, 1.61 mmol) in DMF (1.0 mL) was added, and the mixture was stirred for 3 h. The reaction mixture was diluted with H₂O and extracted with Et₂O. The ether layer was dried over Na₂SO₄, concentrated in vacuo, and the crude mass was purified by SiO₂ chromatography to afford a pale yellow solid ABL7 (1.17 g, 88% yield). AP-ESI+ Mass calcd C₄₈H₄₉N₇O₆: 819.4, Found: 819.8 [M+H]⁺

Preparation of N-α-palmitoyl N-imidazolyl-trityl α-(amido Peg₃ azide) L-histidine (ABL9): N-Fmoc ABL7 (1.17 g, 1.42 mmol) in CH₂Cl₂ (5 mL) was treated with piperidine (0.56 mL) and stirred for 1 h to provide ABL8 of acceptable purity; AP-ESI+ Mass calcd C₃₃H₃₉N₇O₄: 597.3, Found: 597.9 [M+H]⁺. The mixture was concentrated in vacuo, and the residue was washed with hexanes. Crude ABL8 was dissolved in CH₂Cl₂ (5 mL) and treated with palmitic acid (0.73 g, 2.84 mmol), diisopropylcarbodiimide (0.36 g, 2.84 mmol), and NHS (0.43 g, 2.84 mmol). The precipitate was removed by filtration, and the crude product was purified by SiO₂ chromatography to afford off-white solid ABL9 (0.71 g, 60% yield). AP-ESI+ Mass calcd C₄₉H₆₉N₇O₅: 835.5, Found: 835.9 [M+H]⁺

Preparation of N-α-palmitoyl α-(amido Peg₃ azide) L-histidine (ABL10): A solution of N-imidazolyl-trityl ABL9 (0.71 g, 0.85 mmol) and TIPS (0.17 mL, 0.85 mmol) in DCM (2 mL) was treated with TFA (10 mL). After 1.5 h, the mixture was concentrated in vacuo. The oily crude product was washed with hexanes, dried in vacuo, and purified by RP-HPLC to afford ABL10 (394 mg, 79% yield). AP-ESI+ Mass calcd C₃₀H₅₅N₇O₅: 593.4, Found: 594.3 [M+H]⁺

### Synthesis of Oligonucleotide Crosslinking Auxiliary Moiety:

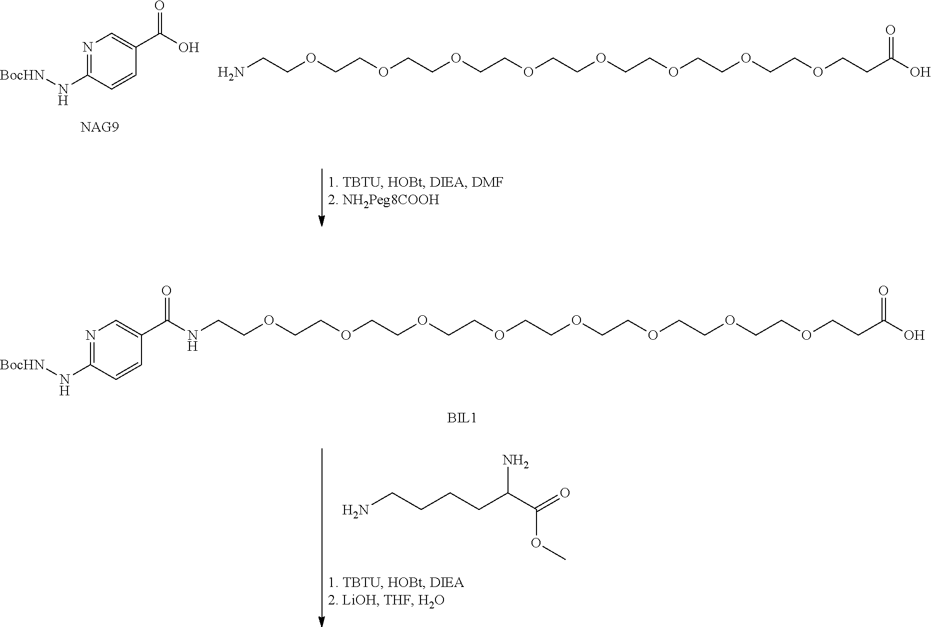 [SMILES: CC(C)(C)OC(=O)NNc1ccc(C(=O)O)cn1 ; NCCOCCOCCOCCOCCOCCOCCOCCOCCC(=O)O ; CC(C)(C)OC(=O)NNc1ccc(C(=O)NCCOCCOCCOCCOCCOCCOCCOCCOCCC(=O)O)cn1 ; COC(=O)C(N)CCCCN]

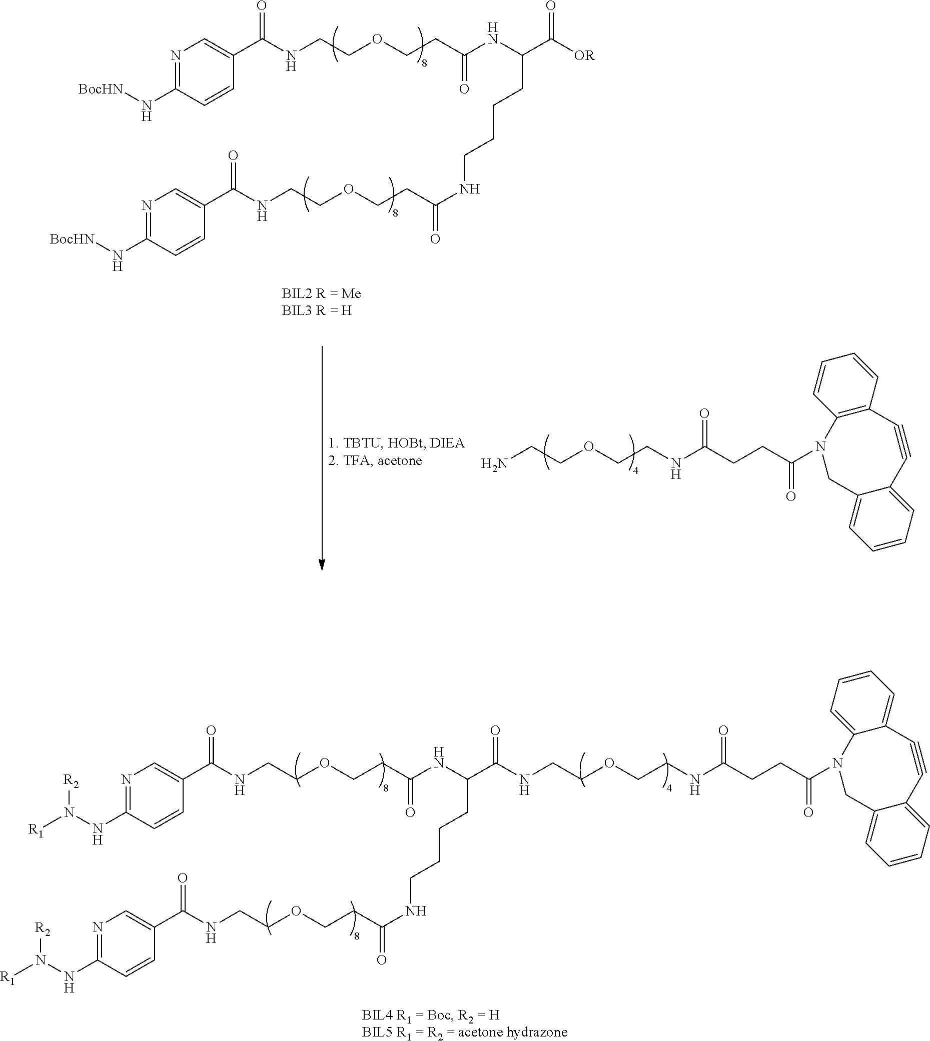

Preparation of 3-[2-(2-{2-[2-(2-{2-[2-(2-(6-[(tert-Butyl)-2-carboxyhydrazino)-nicotinoylamino}ethoxy)ethoxy]ethoxy}ethoxy)ethoxy]ethoxy}ethoxy)ethoxy]propionic acid (BIL1). N-Bo 4-hydrazino nicotinic acid, NAG9, (0.38 g, 1.50 mmol) was activated with TBTU (0.48 g, 1.50 mmol), HOBt (0.23 g, 1.50 mmol), and DIEA (0.39 g, 3.00 mg) in DMF (10 mL). After 20 min, a solution to Peg₈ amino acid (0.44 g, 1.00 mmol) was added, and the reaction was stirred for 1 h at room temperature. The reaction mixture was concentrated in vacuo and purified by SiO₂ chromatography with 5% MeOH in DCM to afford BIL1 (0.39 g, 58% yield). ESI MS+ mass calculated C₃₀H₇₂N₄O₁₃: 676.4, Found: 677.0 [M+H]⁺.

Preparation of methyl (2S)-2,6-bis{3-[2-(2-{2-[2-(2-{2-[2-(2-{6-[(tert-butyl)-2-carboxyhydrazino]-nicotinoylamino}ethoxy)ethoxy]ethoxy}ethoxy)ethoxy]ethoxy}ethoxy)ethoxy]propionylamino}hexanoate (BIL2). BIL1 (0.39 g, 0.57 mmol) was activated with TBTU (0.19 g, 0.58 mmol), HOBt (0.089 g, 0.58 mmol), and DIEA (0.23 mL, 1.30 mmol) in DMF (5 mL). After 15 min, Lys methyl ester hydrochloride was added with additional DIEA (0.23 mL). The reaction was sonicated to fully dissolve the Lys methyl ester. After stirring overnight, the reaction was concentrated in vacuo and purified by SiO₂ chromatography 5-15% MeOH in CH₂Cl₂ to afford BIL2 (0.38 g, 93% yield). ESI MS+ mass calculated C₆₇H₁₁₆N₁₀O₂₆: 1476.8, Found: 1477.0 [M+H]⁺.

Preparation of (2S)-2,6-Bis{3-[2-(2-{2-[2-(2-{2-[2-(2-{6-[(tert-butyl)-2-carboxyhydrazino]-nicotinoylamino}ethoxy)ethoxy]ethoxy}ethoxy)ethoxy]ethoxy}ethoxy)ethoxy]propionylamino}hexanoic acid (BIL3). BIL2 (0.37 g, 0.26 mmol) in THE (30 mL) was saponified through treatment with LiOH.H₂O (0.032 g, 0.75 mmol) in water (2 mL) and MeOH (2 mL). The reaction was stirred at room temperature for 1 h, after which HPLCMS showed complete conversion. The mixture was neutralized with Dowex H+ resin, and the filtrate was concentrated in vacuo to provide BIL3 (0.25 g, 66% yield). This material was sufficiently pure for the next reaction. ESI MS+ mass calculated C₆₆H₁₁₄N₁₀O₂₆: 1462.8, Found: 732.0 [M+H]³⁺ m/3z.

Preparation of (2S)-1-[3-(2-{2-[2-(3-{2-Azatricyclo[10.4.0.0^{4,9}]hexadeca-1(16),4,6,8,12,14-hexaen-10-yn-2-yl}-3-oxopropoxy)ethoxy]ethoxy}ethoxy)propylamino]-2,6-bis[3-(2-{2-[2-(2-{2-[2-(2-{2-[6-(isopropylidenehydrazino)-nicotinoylamino]ethoxy}ethoxy)ethoxy]ethoxy}ethoxy)ethoxy]-ethoxy}ethoxy)propionylamino]-1-hexanone (BIL5). BIL3 (0.14 g, 0.09 mmol) in DMF (3 mL) was treated with TBTU (0.033 g, 0.10 mmol), HOBt (0.016 g, 0.10 mmol), and DIEA (65 μL, 0.38 mmol) in the presence of DBCO-peg₄ amine (0.049 g, 0.09 mmol). The mixture was stirred for 1 h at room temperature, concentrated in vacuo, and purified by SiO₂ chromatography (5% MeOH in CH₂C1₂) to afford BIL4 (0.051 g, 28% yield). ESI MS+ mass calculated C₉₅H₁₄₉N₁₃O₃₁: 1968.0, Found: 884.7 [M+2H-2Boc]2⁺. BIL4 (0.051 g) was treated with TFA (9 mL) and anhydrous acetone (1 mL) for 30. The reaction mixture was concentrated in vacuo and purified by a fast RP-HPLC gradient to provided BIL5-2×TFA salt (0.050 g, 85% yield). MALDI-TOF MS+ mass calculated C₉₁H₁₄₁N₁₃O₂₇: 1849.16, Found: 1850.14 [M+H]⁺.

### Disulfide Phosphotriester Oligonucleotide Synthesis:

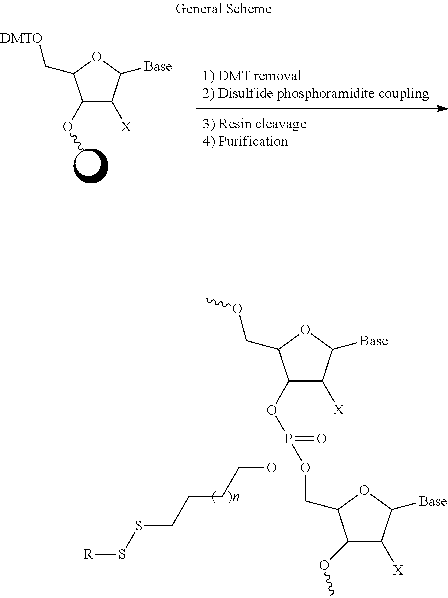

### Experimental Details:

All the oligonucleotide sequences synthesized were modified at 2′-ribose sugar position with 2′-F and 2′-OMe modifications to improve serum stability and to minimize off-target effects. Automated oligonucleotide synthesis (1 μmol scale) was carried out with the following reagents/solvents:

Oxidizer—0.02 M I₂ in THF/Pyridine/H₂O (60 s oxidation per cycle),

Deblock—3% Trichloroacetic Acid (2×40 s deblocks per cycle),

Cap Mix A—THF/Pyridine/Pac₂O (60 s capping per cycle), and

Cap Mix B—16% Methyl imidazole in THE (60 s capping per cycle)

Exceptions to standard oligonucleotide synthesis conditions were as follows:
CPG supports with Q-linkers (hydroquinone-O,O′-diacetic acid linker arm) for milder deprotection were used;
All disulfide phosphoramidites were resuspended to 100 mM in 100% anhydrous acetonitrile prior to synthesis; and
Phosphoramidite activation was performed with 2.5-fold molar excess of 5-benzylthio-1-H-tetrazole (BTT). Activated phosphoramidites were coupled for 2×3 minute coupling steps per insertion.

### Disulfide Phosphotriester Oligonucleotide Deprotection & Purification Protocol:

Following automated oligonucleotide synthesis, disulfide phosphotriester oligonucleotides were cleaved and deprotected in 1 ml of 10% diisopropylamine in methanol (10% DIA/MeOH) for 4 h at room temperature. Following the 4 h deprotection, oligo samples were dried by centrifugal evaporation.
In oligonucleotide synthesis using phosphoramidite monomers having standard protecting groups (such as benzoyl (Bz), acetyl (Ac), and isobutyl (iBu), etc.), the resulting disulfide phosphotriester oligonucleotides were cleaved and deprotected in 1.0 mL of AMA (1:1 ratio of 36% aq. ammonia and 40% methylamine in methanol) for 2 hr at room temperature followed by centrifugal evaporation.
Crude oligo pellets were resuspended in 100 μl of 50% acetonitrile, briefly heated to 65° C. and vortexed thoroughly. Total 100 μl crude oligo samples were injected onto RP-HPLC with the following buffers/gradient:
   Buffer A=50 mM TEAA in Water;
   Buffer B=90% Acetontrile; and
   Flow Rate=1 ml/min;
      Gradient:
         0-2 min (100% Buffer A/0% Buffer B),
         2-42 min (0% to 60% Buffer B), and
         42-55 min (60% to 100% Buffer B).
Across the dominant RP-HPLC peaks, 0.5 ml fractions were collected and analyzed by MALDI-TOF mass spectrometry to confirm presence of desired mass. Purified fractions containing correct mass were frozen and lyophilized. Once dry, fractions were resuspended, combined with corresponding fractions, frozen and lyophilized for final product.

Disulfide insertions requiring additional deprotection were initially isolated as described above followed by the necessary secondary deprotection steps (see below):

### Aldehyde-Disulfide Phosphotriester Secondary Deprotection:

RP-HPLC purified oligo products were resuspended in 100 μl of 80% formic acid. Reaction was allowed to proceed at room temperature for -1 h per aldehyde modification. Reaction was monitored by MALDI-TOF mass spectrometry to confirm complete deprotection. Once deprotection was complete, samples were frozen and lyophilized until dry. Lyophilized samples were then resuspended in 1 ml of 20% acetonitrile and gel-filtered for isolation of final oligo product.

### Hydroxyl-Disulfide Phosphotriester Secondary Deprotection:

RP-HPLC purified oligo products were resuspended in 219 μl of anhydrous DMSO, heated briefly to 65° C. and vortexed thoroughly. To the DMSO solutions, 31 μl of 6.1 M triethylamine trihydrofluoride (TEA.3HF) was added to give a final concentration of 0.75 M. Reaction was allowed to proceed at room temperature for ˜1 h per TBDMS-protected hydroxyl modification. Reaction was monitored by MALDI-TOF mass spectrometry to confirm complete deprotection. Once deprotection was complete, 35 μl of 3 M sodium acetate followed by 1 ml of butanol were added. Samples were vortexed thoroughly and placed at −80° C. for 2 h. After 2 h, samples were centrifuged to pellet oligonucleotides. Butanol layer was removed and the oligo pellet was resuspended in 1 ml of 20% acetonitrile. Samples were gel filtered for isolation of final oligo product.

### Methylphosphonate Containing Oligonucleotide Synthesis:

Methylphosphonate oligonucleotides were synthesized using standard oligo synthesis protocol described herein employing commercially available p-methyl phosphonamidites.

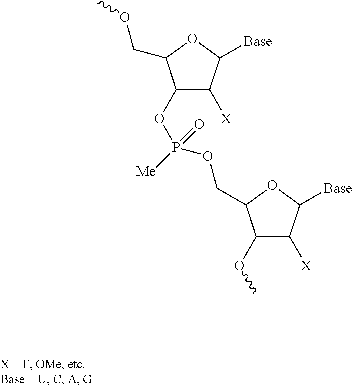

For example, the following commercially available P-Methyl phosphonamidite monomers were used in the synthesis:

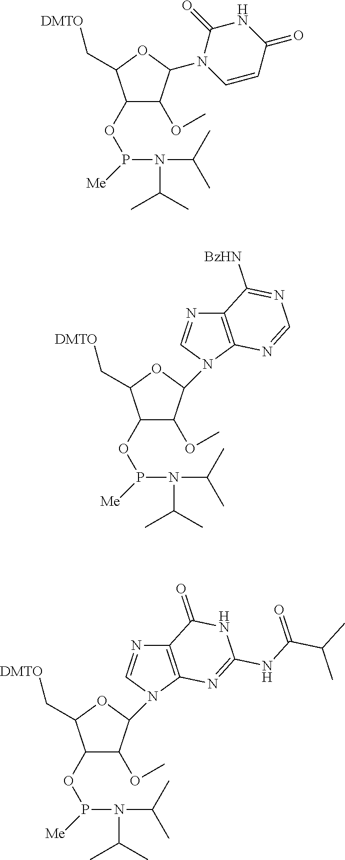

### Phosphoramidate Containing Oligonucleotide Synthesis:

Phosphoramidate oligonucleotide of the following general formula was from the corresponding phosphite (H-phosphonate) and amine.

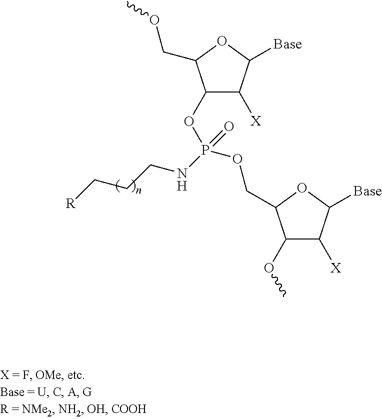

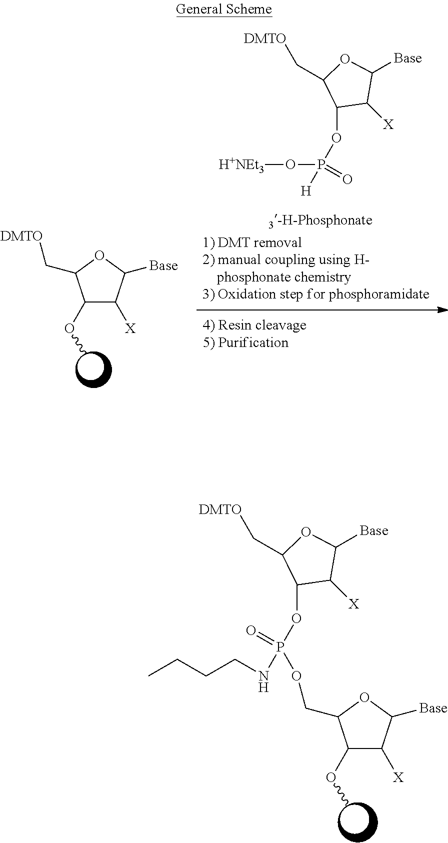

### Experimental Details:

All prepared oligonucleotides include 2′-F or 2′-OMe modified riboses to improve serum stability and to minimize off-target effects. Automated oligonucleotide synthesis (1 μmol scale) was carried out using the following steps:
Deprotection—3% Trichloroacetic Acid (2×40 s deblocks per cycle)
Coupling—1:1 of Pivaloyl Chloride (0.5M) and 3′-H-phosphonate (0.1M) in anhydrous acetonitrile:pyridine (1:1) in x2 times
Oxidation step—4.5:4.5:1 of CCl₄/pyridine/n-butylamine (manually, 2×120s per cycle)
Cap Mix A—THF/pyridine/Pac₂O (60 s capping per cycle)
Cap Mix B—16% methyl imidazole in THE (60 s capping per cycle)
   Exceptions to standard oligonucleotide synthesis conditions were as follows:
CPG supports with Q-linkers (hydroquinone-O,O′-diacetic acid linker arm) for milder deprotection were used;
Protected 3′-H-phosphonates were resuspended to 100 mM in 1:1 of anhydrous acetonitrile and pyridine prior to synthesis;
Pivaloyl chloride was dissolved to give 500 mM solution in 1:1 of anhydrous acetonitrile and pyridine prior to synthesis
Coupling step was carried out manually, the protected 3′-H-phosphonate activation was performed with 5.0-fold molar excess of pivaloyl chloride. The coupling step was carried out for 2×5 minute coupling steps per insertion
Phosphoramidate linkages were obtained by the oxidation step with 90:90:20 μL of anhydrous CCl₄: pyridine: n-butylamine, for 2×2 min cycle

### Disulfide Phosphotriester Oligonucleotide Deprotection & Purification Protocol:

Following automated oligonucleotide synthesis, disulfide phosphotriester oligonucleotides were cleaved and deprotected in 1 ml of 10% diisopropylamine in methanol (10% DIA/MeOH) for 4 h at room temperature. Following the 4 h deprotection, oligo samples were dried by centrifugal evaporation.
Oligo synthesis using phosphoramidite and 3′H-phosphonate monomers having standard protecting groups (such as A-Bz, C—Ac and G-iBu (isobutyrate) etc.), phosphoramidate oligonucleotides were cleaved and deprotected in 1.0 mL of AMA (1:1 ratio of 36% aq. Ammonia and 40% Methylamine in Methanol) for 2 hr at room temperature followed by centrifugal evaporation.
Crude oligo pellets were resuspended in 100 μL of 50% acetonitrile, briefly heated to 65° C., and vortexed thoroughly. Total 100 all crude oligo samples were injected onto RP-HPLC with the following buffers/gradient:
   Buffer A=50 mM TEAA in Water
   Buffer B=90% Acetonitrile
   Flow Rate=1 ml/min
      o Gradient:
         0-2 min (100% Buffer A/0% Buffer B)
         2-42 min (0% to 60% Buffer B)
         42-55 min (60% to 100% Buffer B)
Across the dominant RP-HPLC peaks, 0.5 ml fractions were collected and analyzed by MALDI-TOF mass spectrometry to confirm presence of desired mass. Purified fractions containing correct mass were frozen and lyophilized. Once dry, fractions were re-suspended, combined with corresponding fractions, frozen and lyophilized for final product.

### Disulfide Phosphotriester Oligonucleotide Conjugation Through Condensation Reaction—General Protocol (See Conjugation General Schemes 1-3):

Disulfide phosphotriester duplexes were generated by equimolar mixing of desired passenger and guide strand oligos. Following the addition of sodium chloride to a final concentration of 50 mM, samples were heated to 65° C. for 5 minutes and allowed to cool to room temperature to complete annealing.
For aldehyde-modified disulfide phoshotriester oligos, siRNA duplexes were diluted into 1× conjugation buffer prior to the addition of the desired HyNic conjugation moiety.

### Conjugation Buffer: 10 mM HEPES (pH 5.5), 20 mM Aniline, 50 mM NaCl, 50% Acetonitrile

Once the above reaction was mixed, a two-fold molar excess of HyNic conjugation component was added to the mixture. Reaction was allowed to proceed at room temperature for 1 h.
After 1 h, conjugated siRNA oligonucleotides were isolated by either gel filtration, HPLC purification or centrifugal spin filtration for final products prior to cellular treatment.

### Disulfide Phosphotriester Oligonucleotide Conjugation Through Click Reaction—General Protocol (See Conjugation General Schemes 4-9):

### Copper-THPTA Complex Preparation:

A 5 mM aqueous solution of copper sulfate pentahydrate (CuSO₄-5H₂O) and a 10 mM aqueous solution of Tris(3-hydroxypropyltriazolylmethyl)amine (THPTA) were mixed 1:1 (v/v) (1:2 molar ratio) and allowed to stand at room temperature for 1 hour. This complex can be used to catalyze Huisgen cycloaddition for example See General Conjugation Schemes 4 and 5.

### Click Reaction (100 nM Scale)

To a solution of 710 uL of water and 100 uL tert-butanol (10% of final volume) in a 1.7 mL eppendorf tube was added 60 uL of the copper-THPTA complex followed by 50 uL of a 2 mM solution of the oligo, 60 ul of a 20 mM aqueous sodium ascorbate solution and 20 uL of a 10 mM solution of GaINAc-azide. After thorough mixing the solution was allowed to stand at room temperature for 1 hour. Completion of the reaction was confirmed by gel analysis.

The reaction mixture is added to a screw cap vial containing 5-10 fold molar excess of SiliaMetS®TAAcONa (resin bound EDTA sodium salt). The mixture is stirred for 1 hour. This mixture is then eluted through an Illustra™Nap™-10 column Sephadex™. The solution is then frozen and lyophilized overnight.

### Metal-Free Click Reaction

[3+2] cycloaddition was also performed with DBCO moiety using methods known in the art without the use of copper (see, e.g., Jewett and Bertozzi, Chem. Soc. Rev., 39:1272-1279, 2010).

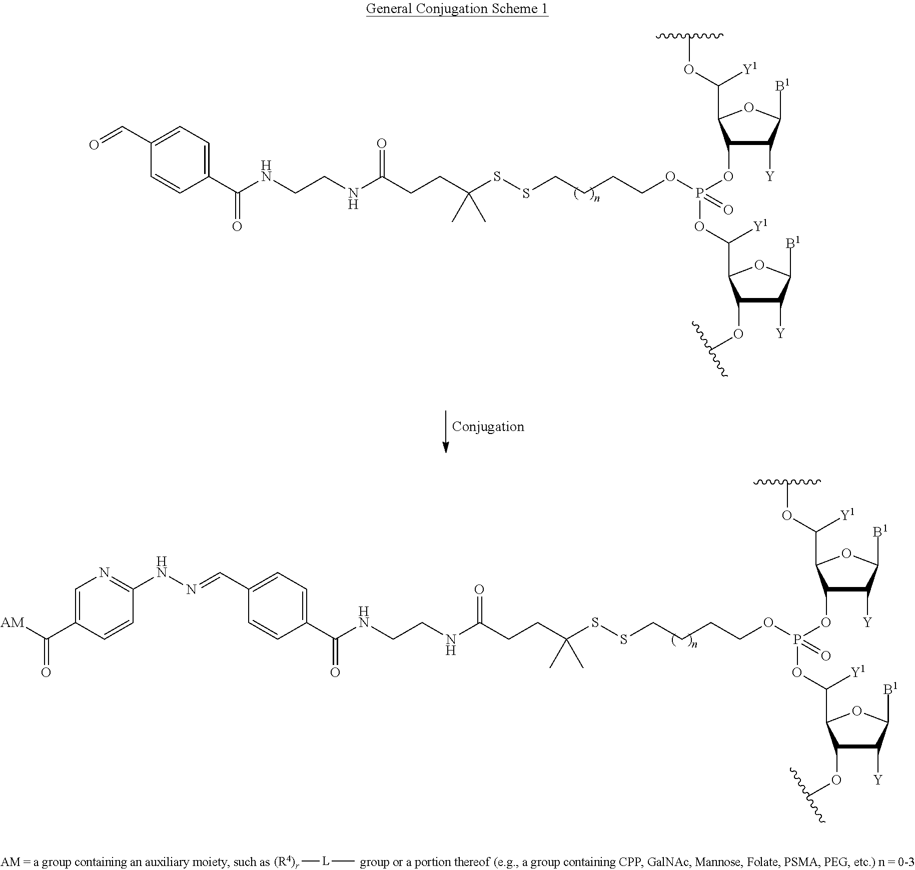

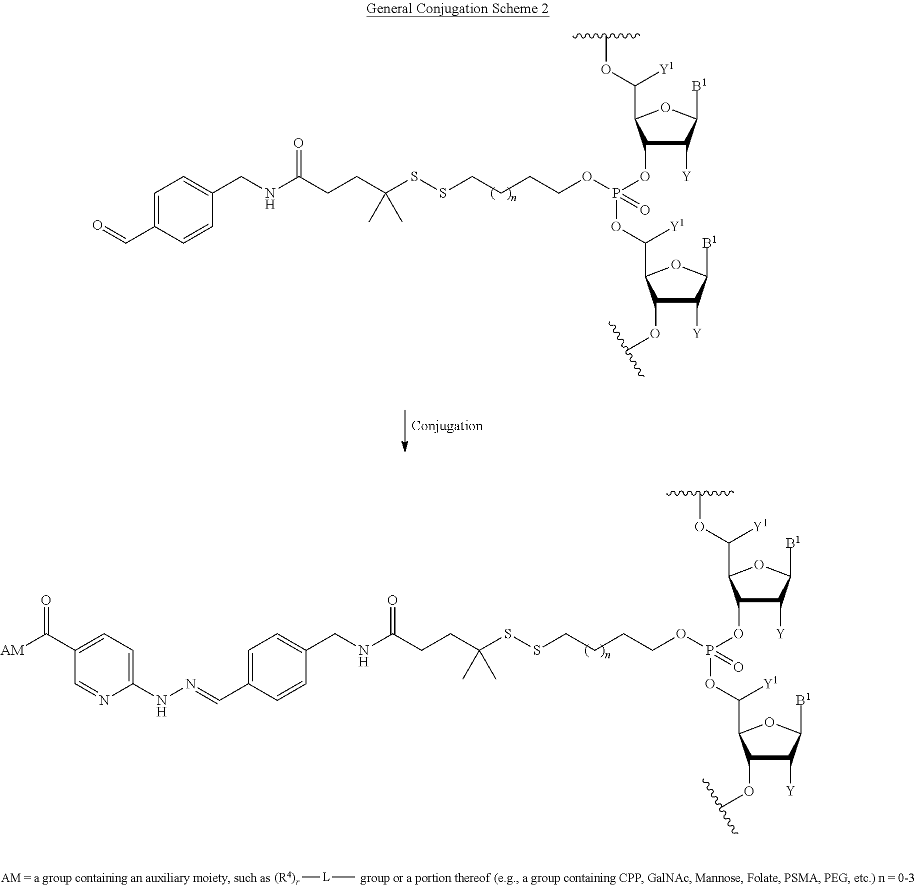

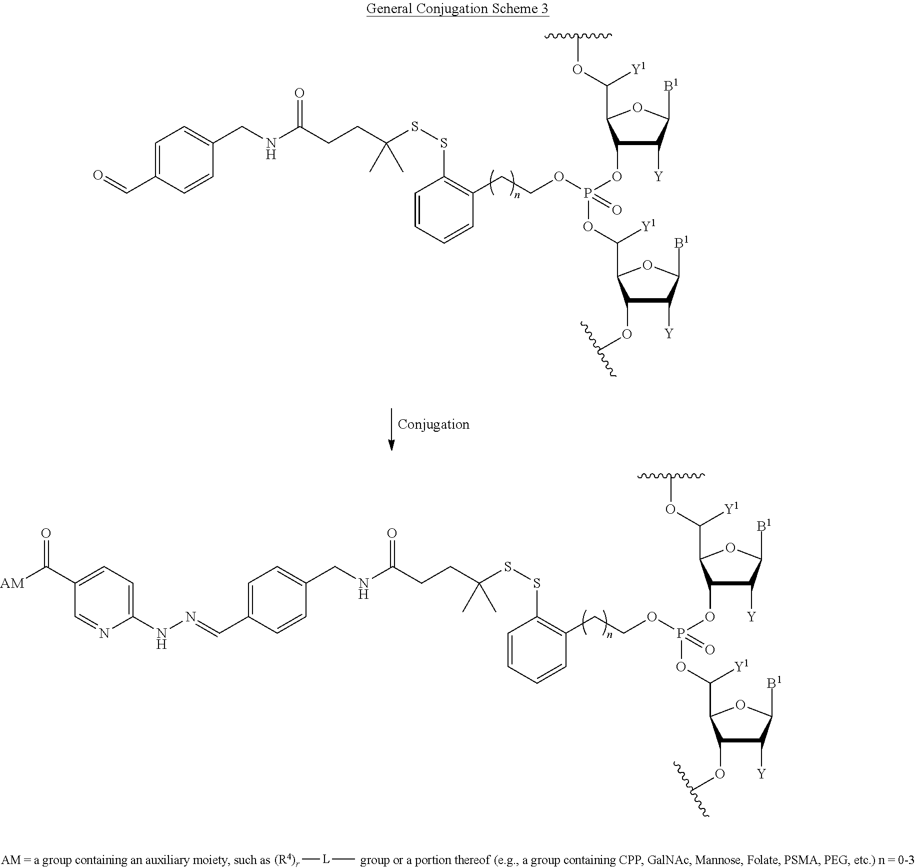

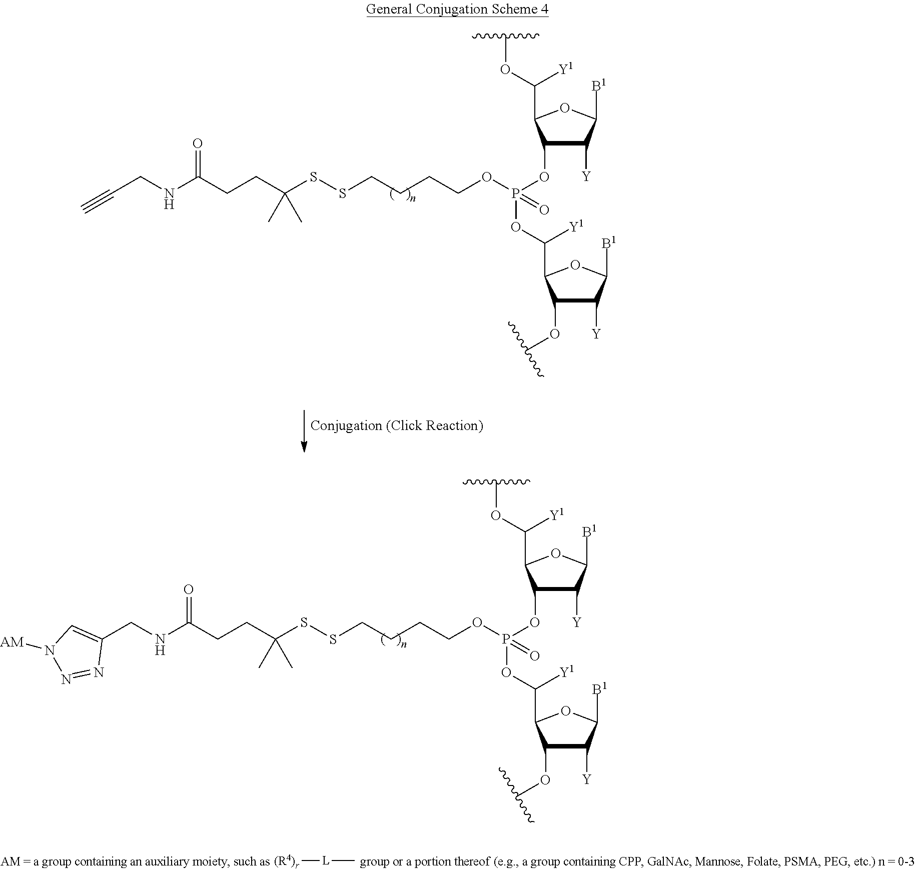

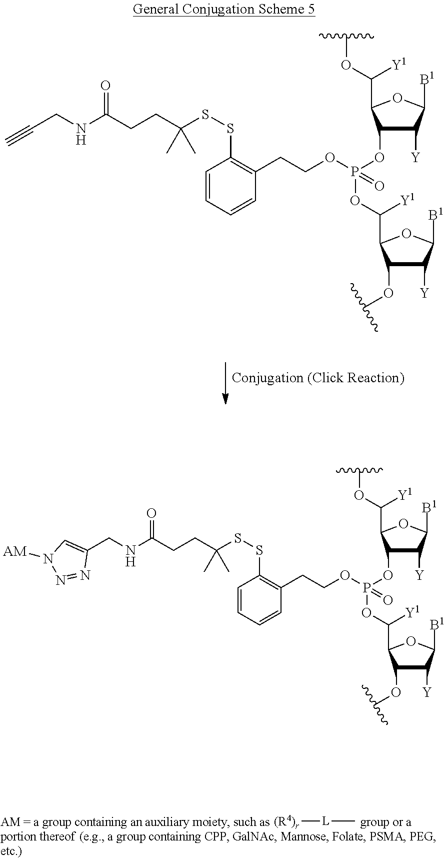

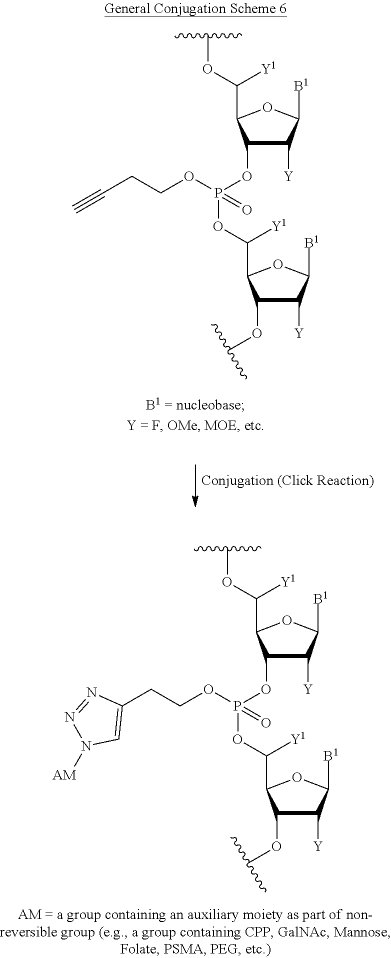

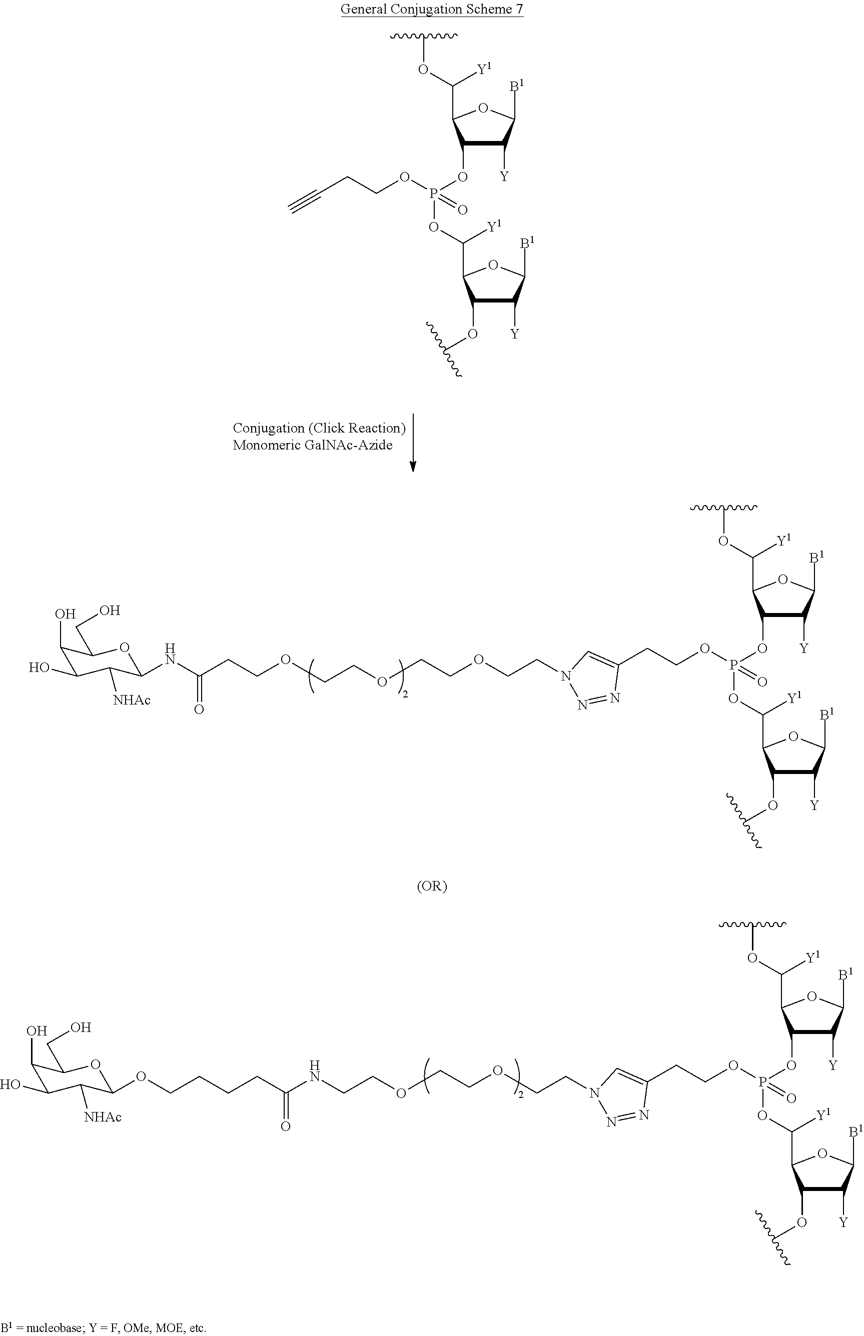

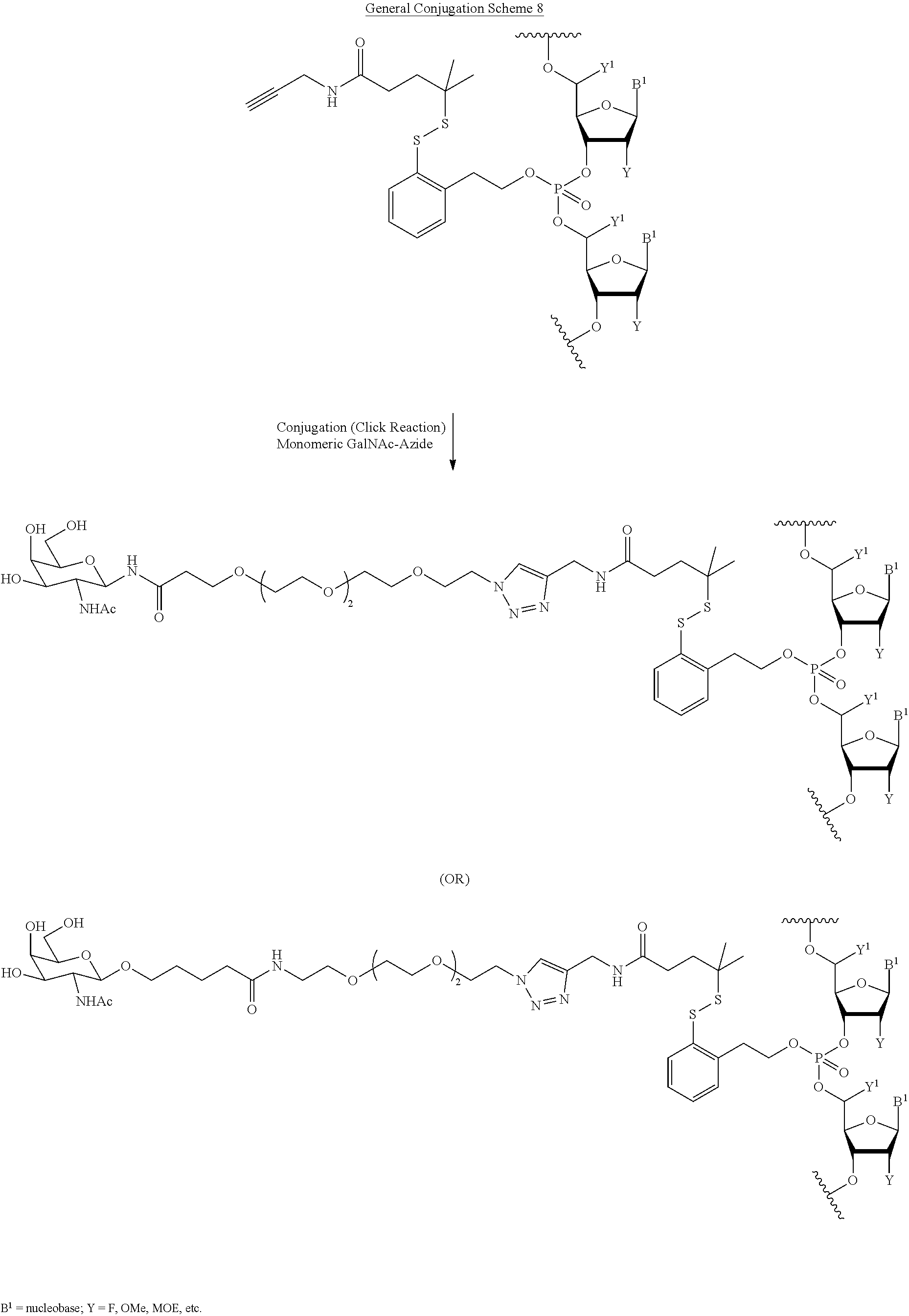

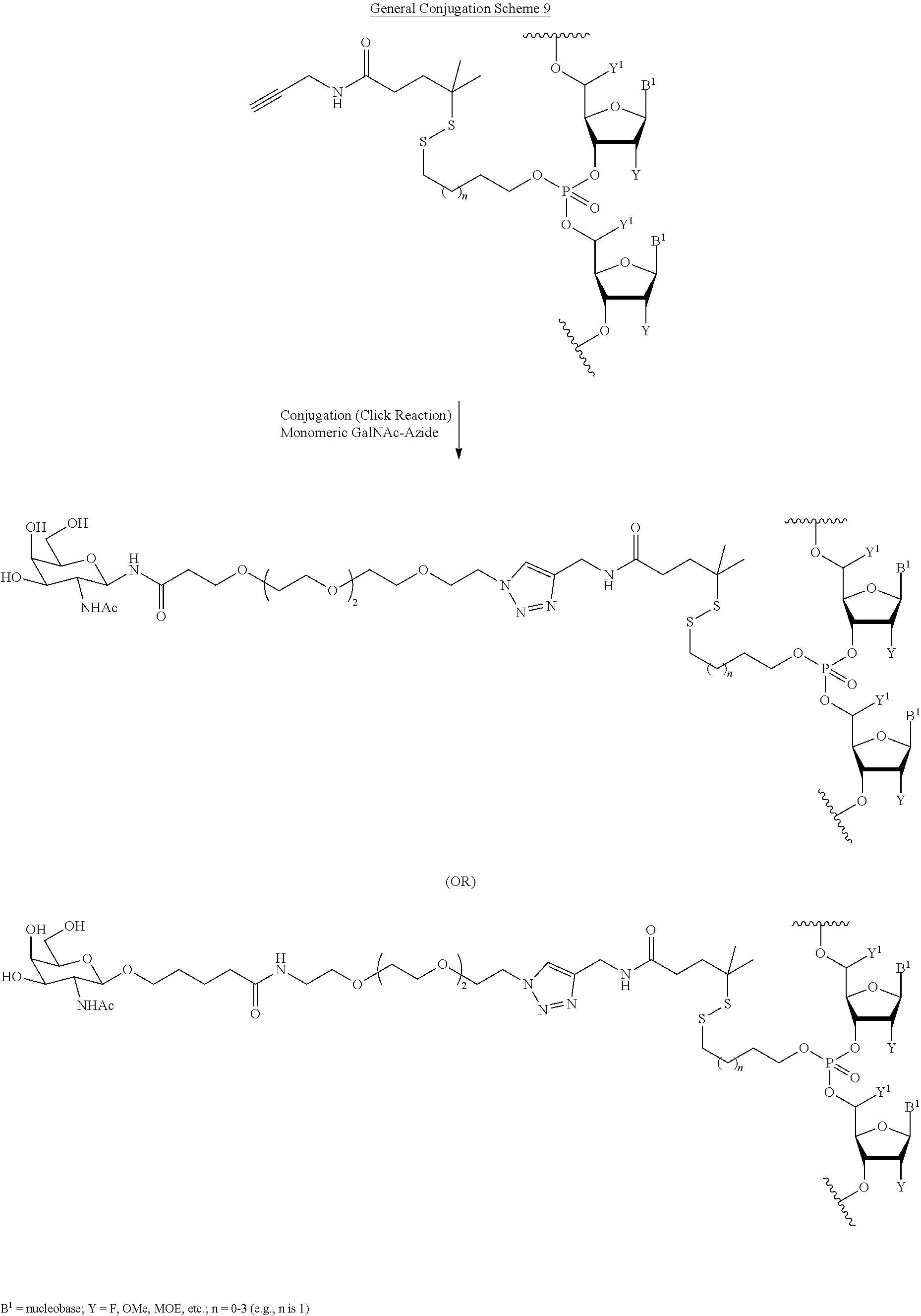

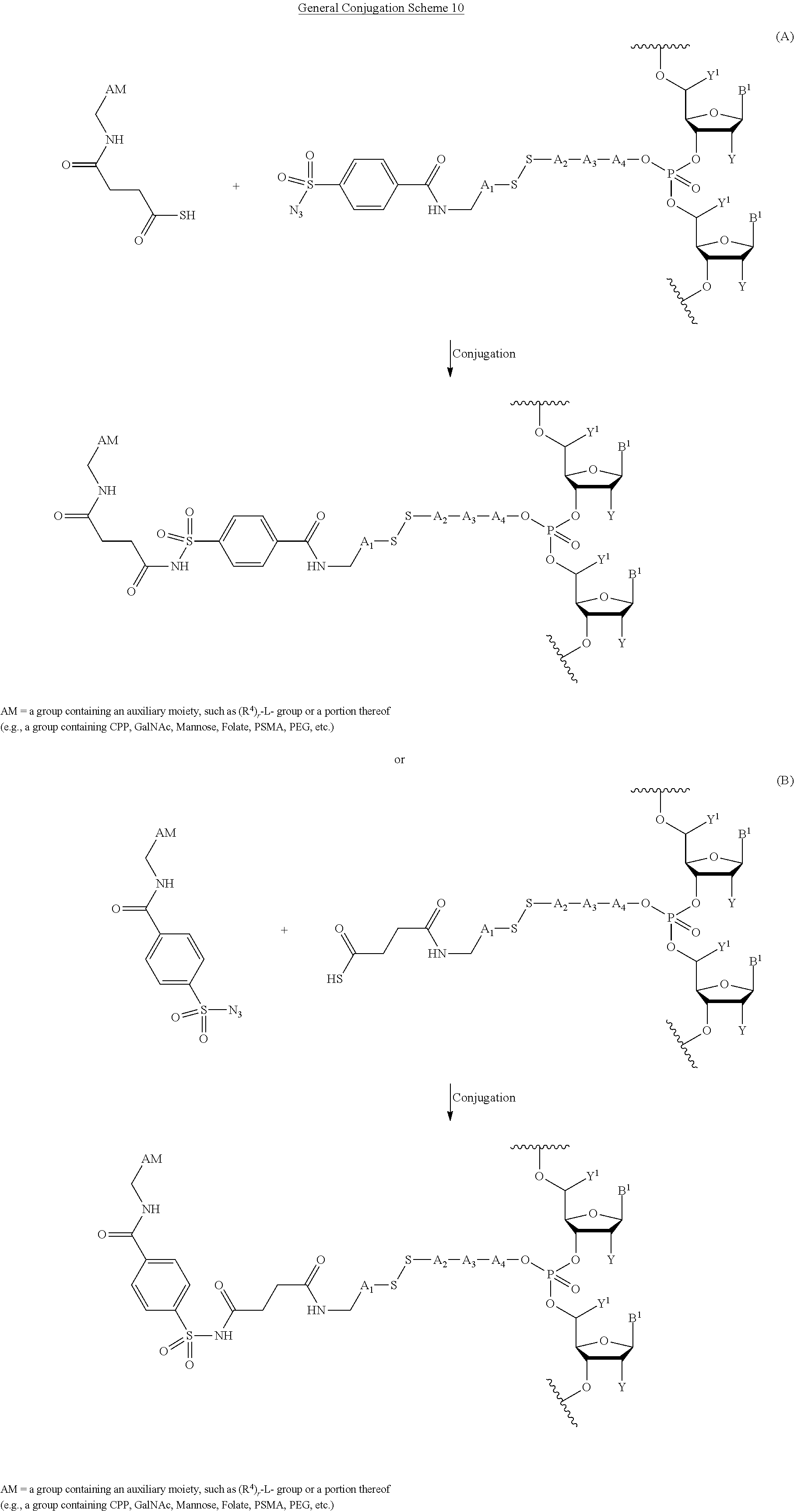

The conjugation schemes described herein are also applicable to non-bioreversible groups and differ from those showing bioreversible groups in that the non-bioreversible groups do not include the disulfide.

### Specific Syntheses of the Polynucleotides of the Invention

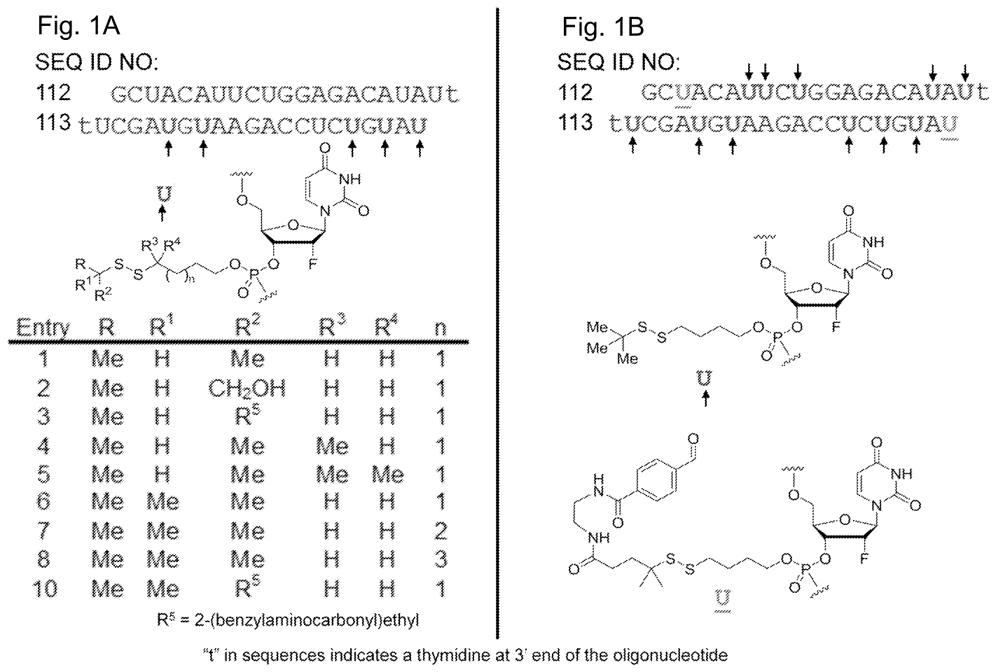

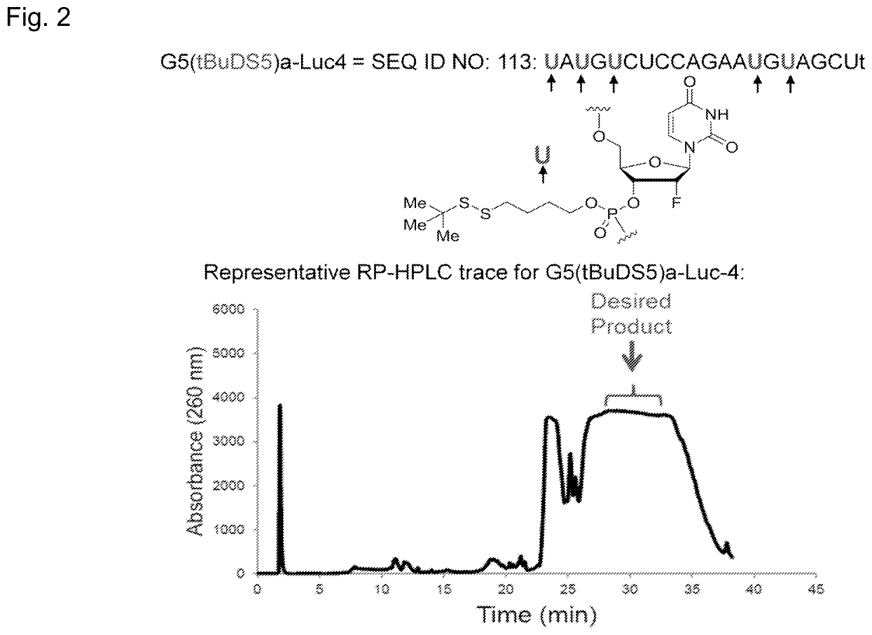

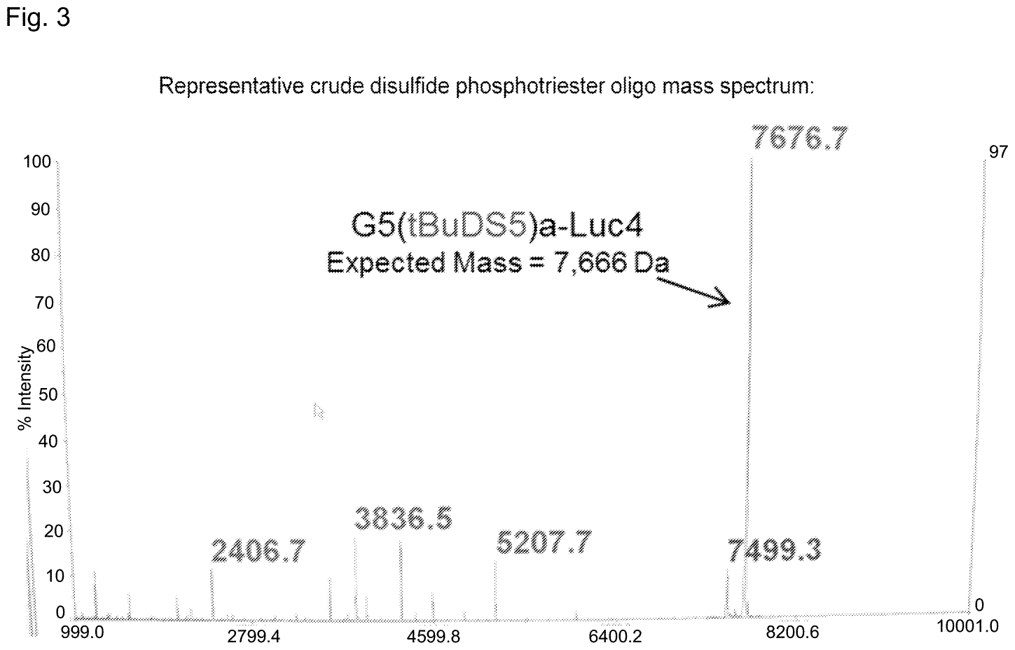

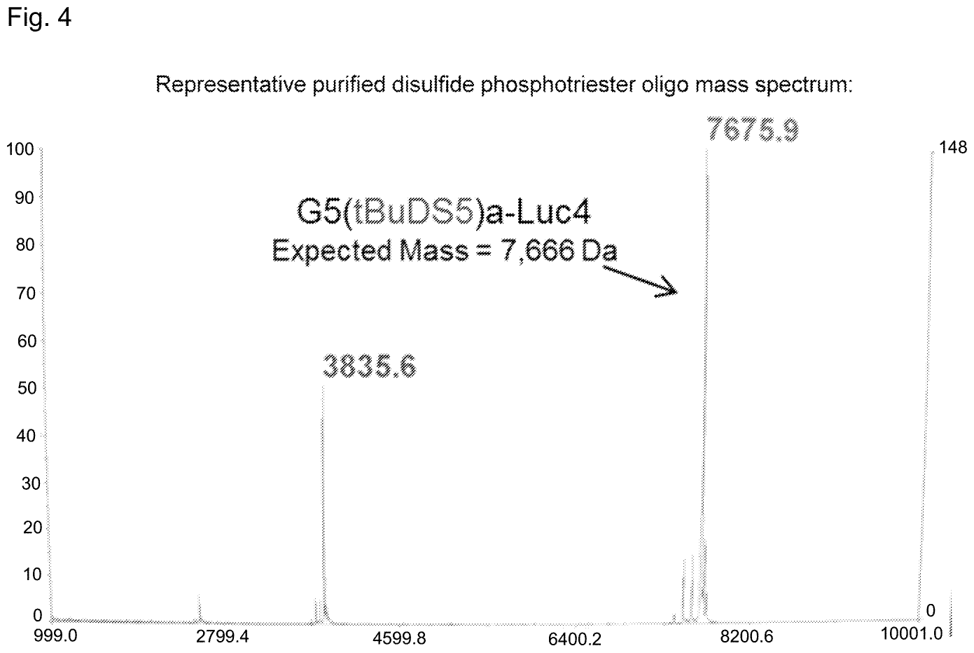

Polynucleotides of the invention have been prepared according to methods described herein. The exemplary polynucleotides are siRNA constructs having the sequences in FIG. 1A or the sequences in FIG. 1B (SEQ ID NOs:112 and 113). Exemplary RP-HPLC trace of SEQ ID NO: 113 is shown in FIG. 2. The mass spectrum of the crude reaction mixture containing the oligonucleotide having the sequence of SEQ ID NO: 113 is shown in FIG. 3. The mass spectrum of the purified oligonucleotide having the sequence of SEQ ID NO: 113 is shown in FIG. 4.

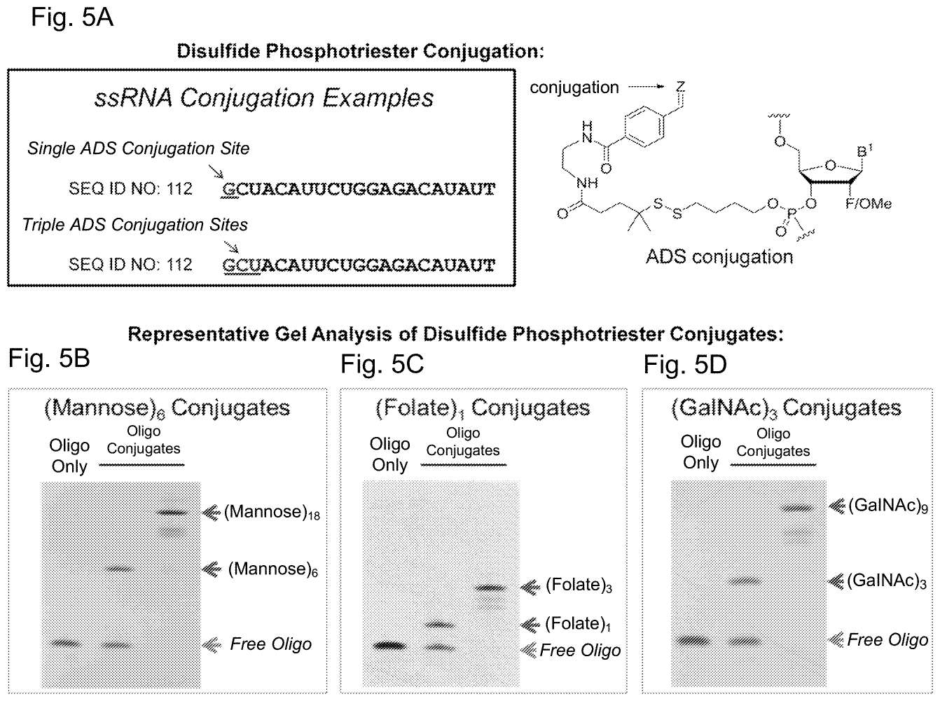

Other polynucleotides of the invention have been prepared according to the methods described herein. For example, FIG. 5A shows ssRNAs having the sequence SEQ ID NO: 112, the single ADS conjugation ssRNA contains one 5′-terminal ADS conjugation site having the structure of “ADS conjugation,” and the triple ADS conjufation ssRNA contains three ADS conjugation sites, each having the structure of “ADS conjugation.” FIGS. 5B-5D show gel analyses of some of the polynucleotides of the invention having one or three nucleotides with conjugated targeting moieties contained in Z of the ADS conjugation structure.

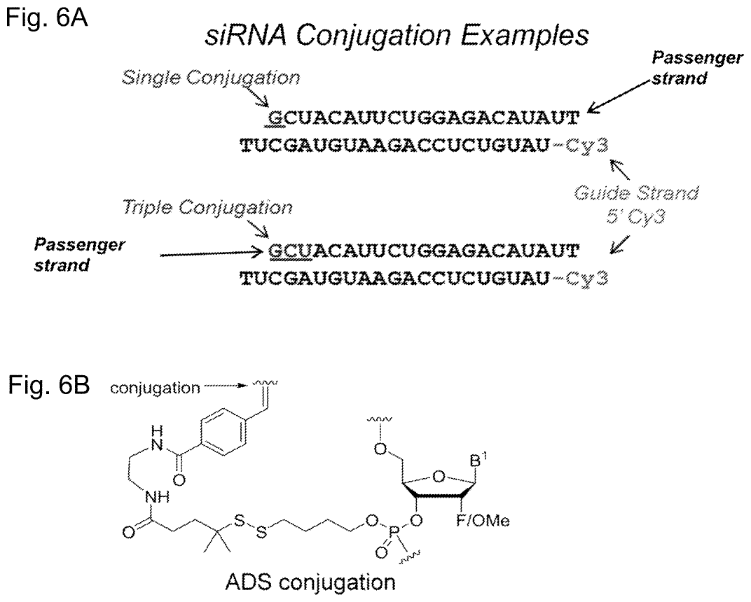

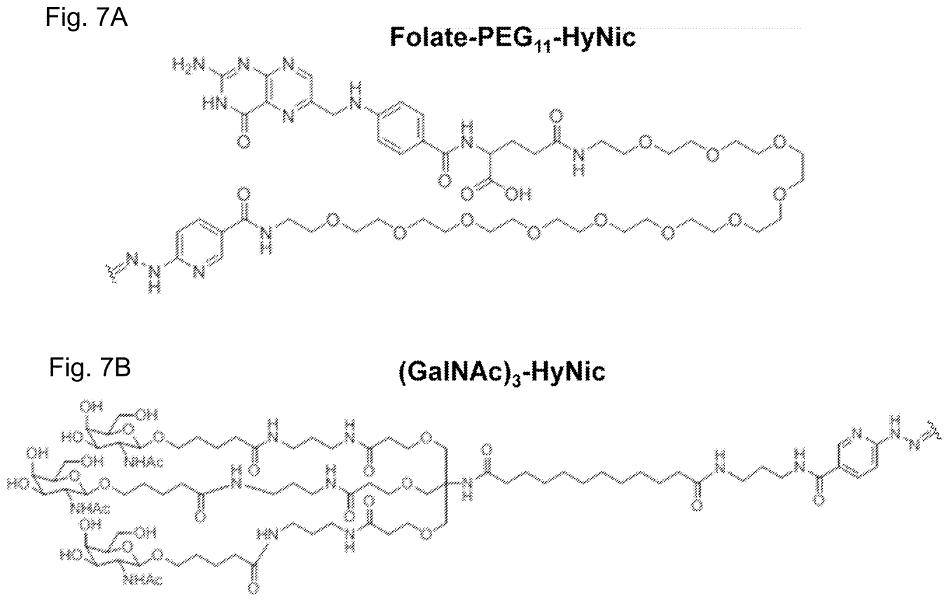

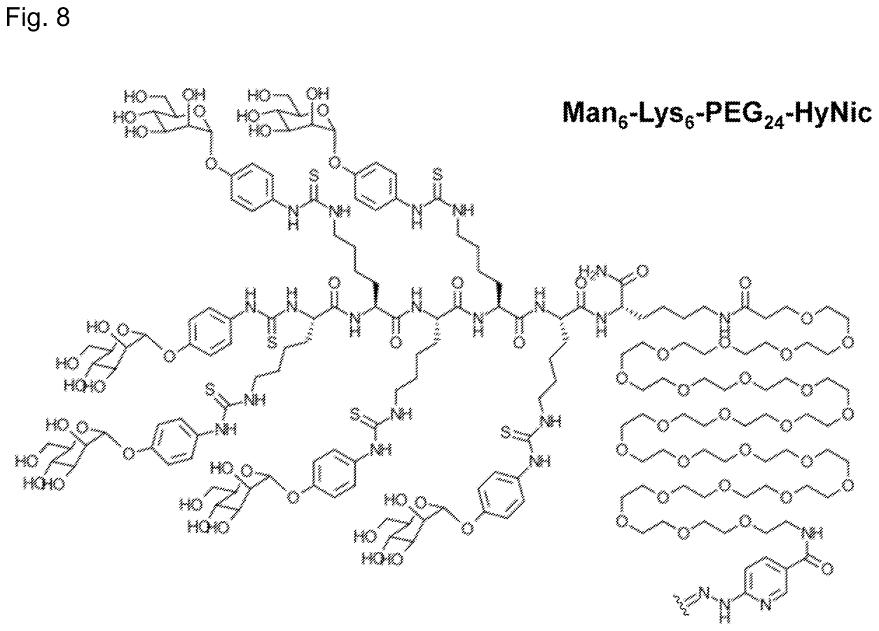

The general structures of the prepared siRNA molecules containing a passenger strand having one or three groups containing targeting moieties are shown in FIGS. 6A and 6B. The guide strand in FIG. 6A has a 5′-terminal Cy3 moiety. Two exemplary polynucleotides of the invention contain one or three Folate-PEG-HyNic groups shown in FIG. 7A. (Folate)₁-siRNN-Cy3 is a polynucleotide construct having a sequence 5′-GCUACAUUCUGGAGACAUAUt (lower-case t is thymidine; SEQ ID NO:112) containing one Folate-PEG-HyNic group conjugated to the internucleotide bridging group of 5′-terminal G. (Folate)₃-siRNN-Cy3 is a polynucleotide construct having a sequence 5′-GCUACAUUCUGGAGACAUAUt containing three Folate-PEG-HyNic groups conjugated to the three internucleotide bridging groups of 5′-GCU. Two exemplary polynucleotides of the invention contain one or three (GaINAc)₃-HyNic groups shown in FIG. 7B. (Ga/NAc)₃-siRNN-Cy3 is a polynucleotide construct having a sequence 5′-GCUACAUUCUGGAGACAUAUt containing one (GaINAc)₃-HyNic group conjugated to the internucleotide bridging group of 5′-terminal G. (Ga/NAc)₉-siRNN-Cy3 is a polynucleotide construct having a sequence 5′-GCUACAUUCUGGAGACAUAUT containing three (GaINAc)₃-HyNic groups conjugated to the three internucleotide bridging groups of 5′-GCU. Two exemplary polynucleotides of the invention contain one or three Man₆-Lys₆-PEG₂₄-HyNic groups shown in FIG. 8. (Mannose)₆-siRNN-Cy3 is a polynucleotide construct having a sequence 5′-GCUACAUUCUGGAGACAUAUT containing one Man₆-Lys₆-PEG₂₄-HyNic group conjugated to the internucleotide bridging group of 5′-terminal G. (Mannose)₁₈-siRNN-Cy3 is a polynucleotide construct having a sequence 5′-GCUACAUUCUGGAGACAUAUT containing a 5′-terminal bioreversible group and two internucleotide bioreversible groups within 5′-GCU, each of the bioreversible groups including Man₆-Lys₆-PEG₂₄-HyNic groups conjugated to the three internucleotide groups of.

Other prepared polynucleotides of the invention contain one to three GaINAc monomers (see below) conjugated to one to ten (e.g., one to four) internucleotide groups as part of non-bioreversible or bioreversible groups.

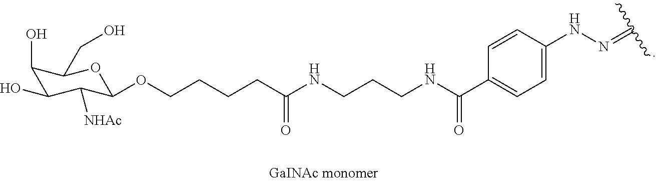 [SMILES: *=NNc1ccc(C(=O)NCCCNC(=O)CCCCOC2OC(CO)C(O)C(O)C2NC(C)=O)cc1]

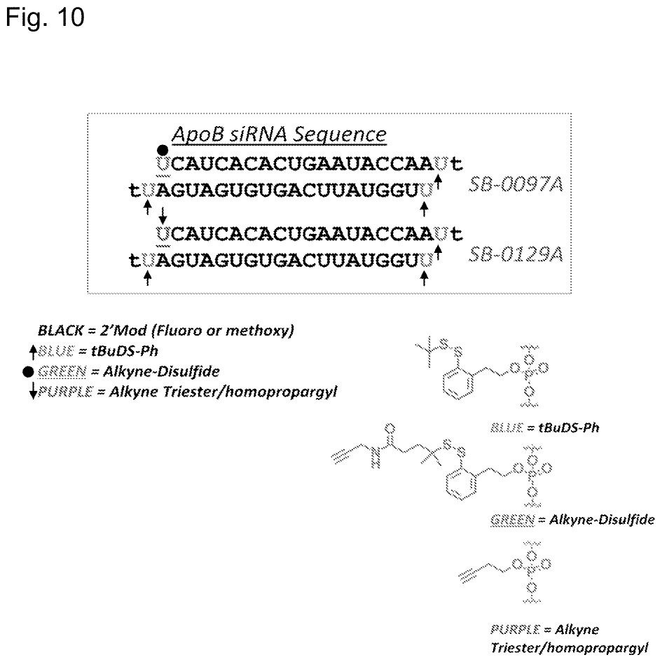

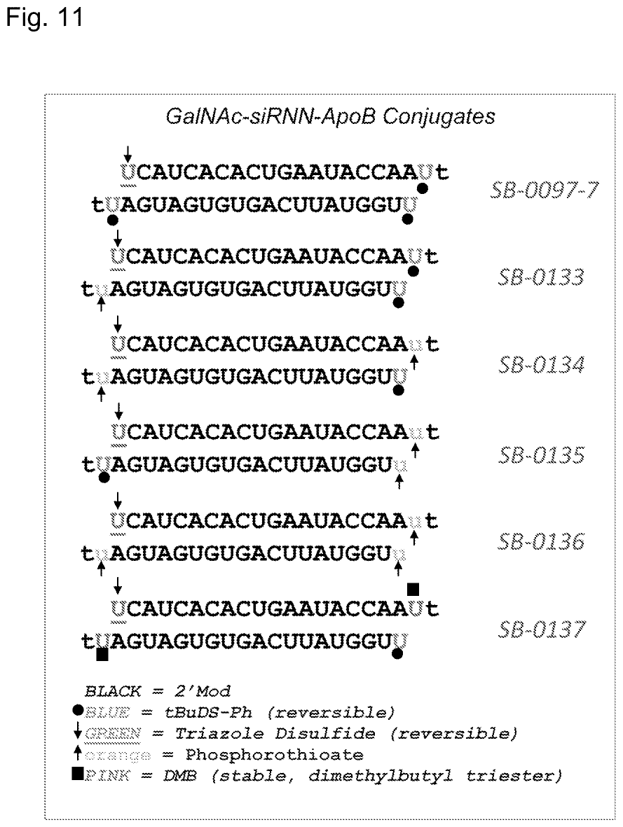

The list of exemplary siRNA triesters and conjugates is provided in Tables 5-9 and in FIGS. 10, 11, and 20A.

TABLE 5

| Compound # | Ligand | Target | Strand | Strand # | Sequences (5′ - 3′) | Conjugation-Prodrug Unker |
|---|---|---|---|---|---|---|
| SB-0068 | P20 | GAPDH | P | P3271 | UCUACAUGUUCCAGUAUGAUt | Aldehyde-Disulfide (4-carbon) |
| | | | G | G3273 | UCAUACUGGAACAUGUAGAUt | Aldehyde-Disulfide (4-carbon) |
| SB-0069 | P21 | GAPDH | P | P3271 | UCUACAUGUUCCAGUAUGAUt | Aldehyde-Disulfide (4-carbon) |
| | | | G | G3273 | UCAUACUGGAACAUGUAGAUt | Aldehyde-Disulfide (4-carbon) |
| SB-0070 | P36 | GAPDH | P | P3271 | UCUACAUGUUCCAGUAUGAUt | Aldehyde-Disulfide (4-carbon) |
| | | | G | G3273 | UCAUACUGGAACAUGUAGAUt | Aldehyde-Disulfide (4-carbon) |
| SB-0071 | M6 | GAPDH | P | P3270 | UCUACAUGUUCCAGUAUGAUt | Aldehyde-Disulfide (4-carbon) |
| | | | G | G3102 | UCAUACUGGAACAUGUAGAUt | |
| SB-0072 | M6 | GAPDH | P | P3271 | UCUACAUGUUCCAGUAUGAUt | Aldehyde-Disulfide (4-carbon) |
| | | | G | G3102 | UCAUACUGGAACAUGUAGAUt | |
| SB-0073 | NAG19 | ApoB | P | P3276 | Ald-UCAUCACACUGAAUACCAAUt | 5′Hydrazone |
| | | | G | G3258 | UUGGUAUUCAGUGUGAUGAUt | |
| SB-0074 | NAG19 | ApoB | P | P3277 | UCAUCACACUGAAUACCAAUt | Aldehyde-Disulfide (ortho) |
| | | | G | G3258 | UUGGUAUUCAGUGUGAUGAUt | |
| SB-0075 | NAG19 | ApoB | P | P3279 | UCAUCACACUGAAUACCAAUt | Aldehyde-Disulfide (ortho) |
| | | | G | G3282 | UUGGUAUUCAGUGUGAUGAUt | |
| SB-0076 | F3 | GAPDH | P | P3270 | UCUACAUGUUCCAGUAUGAUt | Aldehyde-Disulfide (4-carbon) |
| | P17 | | G | G3272 | UCAUACUGGAACAUGUAGAUt | Aldehyde-Disulfide (4-carbon) |
| SB-0077 | F3 | GAPDH | P | P3270 | UCUACAUGUUCCAGUAUGAUt | Aldehyde-Disulfide (4-carbon) |
| | | | G | G3102 | UCAUACUGGAACAUGUAGAUt | |
| SB-0078 | | GAPDH | P | P3101 | UCUACAUGUUCCAGUAUGAUt | |
| | P17 | | G | G3272 | UCAUACUGGAACAUGUAGAUt | Aldehyde-Disulfide (4-carbon) |
| SB-0080 | F3 | GAPDH | P | P3270 | UCUACAUGUUCCAGUAUGAUt | Aldehyde-Disulfide (4-carbon) |
| | P33 | | G | G3272 | UCAUACUGGAACAUGUAGAUt | Aldehyde-Disulfide (4-carbon) |
| SB-0081 | NAG21 | ApoB | P | P3287 | Hex-UCAUCACACUGAAUACCAAUt | 5′Click |
| | | | G | G3282 | UUGGUAUUCAGUGUGAUGAUt | |
| SB-0082 | F3 | GAPDH | P | P3270 | UCUACAUGUUCCAGUAUGAUt | Aldehyde-Disulfide (4-carbon) |
| | P35 | | G | G3272 | UCAUACUGGAACAUGUAGAUt | Aldehyde-Disulfide (4-carbon) |
| SB-0083 | F3 | GAPDH | P | P3270 | UCUACAUGUUCCAGUAUGAUt | Aldehyde-Disulfide (4-carbon) |
| | P32 | | G | G3272 | UCAUACUGGAACAUGUAGAUt | Aldehyde-Disulfide (4-carbon) |
| SB-0085 | NAG21 | ApoB | P | P3297 | Hex-UCAUCACACUGAAUACCAAUt | 5′Click |
| | | | G | G3258 | UUGGUAUUCAGUGUGAUGAUt | |
| SB-0088 | P34 | GAPDH | P | P3270 | UCUACAUGUUCCAGUAUGAUt | Aldehyde-Disulfide (4-carbon) |
| | | | G | G3102 | UCAUACUGGAACAUGUAGAUt | |
| SB-0089 | P34 | GAPDH | P | P3270 | UCUACAUGUUCCAGUAUGAUt | Aldehyde-Disulfide (4-carbon) |
| | P34 | | G | G3272 | UCAUACUGGAACAUGUAGAUt | Aldehyde-Disulfide (4-carbon) |
| SB-0090 | F3 | GAPDH | P | P3270 | UCUACAUGUUCCAGUAUGAUt | Aldehyde-Disulfide (4-carbon) |
| | P34 | | G | G3272 | UCAUACUGGAACAUGUAGAUt | Aldehyde-Disulfide (4-carbon) |
| SB-0094 | NAG21 | ApoB | P | P3290 | Hex-uCAUCACACUGAAUACCAAut | 5′Click |
| | | | G | G3292 | uUGGUAUUCAGUGUGAUGAut | |
| SB-0095 | NAG21 | NTC | P | P3291 | Hex-AGUACUGCUUACGAUACGGut | 5′Click |
| | | | G | G3293 | CCGUAUCGUAAGCAGUACUut | |
| SB-0096 | NAG19 | ApoB | P | P3294 | UCAUCACACUGAAUACCAAut | Aldehyde-Disulfide (ortho) |
| | | | G | G3292 | uUGGUAUUCAGUGUGAUGAut | |
| SB-0097 | NAG21 | ApoB | P | P3295 | UCAUCACACUGAAUACCAAut | Alkyne-Disulfide (ortho) |
| | | | G | G3292 | uUGGUAUUCAGUGUGAUGAut | |
| SB-0098 | NAG19 | ApoB | P | P3296 | Ald-uCAUCACACUGAAUACCAAut | 5′Hydrazone |
| | | | G | G3292 | uUGGUAUUCAGUGUGAUGAut | |
| SB-0099 | PSMA10 | Luc | P | P3182 | GCUACAUUCUGGAGACAUAUt | Aldehyde-Disulfide (4-carbon) |
| | | | G | G3247 | Cy3-UAUGUCUCCAGAAUGUAGCUt | |
| SB-0100 | PSMA20 | Luc | P | P3182 | GCUACAUUCUGGAGACAUAUt | Aldehyde-Disulfide (4-carbon) |
| | | | G | G3247 | Cy3-UAUGUCUCCAGAAUGUAGCUt | |
| SB-0101 | NAG21 | ApoB | P | P3298 | Hex-UCAUCACACUGAAUACCAAUt | 5′Click |
| | | | G | G3299 | UUGGUAUUCAGUGUGAUGAUt | |
| SB-0102 | NAG21 | ApoB | P | P3307 | uCAUCACACUGAAUACCAAut | Alkyne-Disulfide (ortho) |
| | | | G | G3292 | uUGGUAUUCAGUGUGAUGAut | |
| SB-0103 | NAG21 | ApoB | P | P3308 | uCAUCACACUGAAUACCAAUt | Alkyne-Disulfide (ortho) |
| | | | G | G3292 | uUGGUAUUCAGUGUGAUGAut | |
| SB-0104 | NAG21 | ApoB | P | P3309 | UCAUCACACUGAAUACCAAUt | Alkyne-Disulfide (ortho) |
| | | | G | G3292 | uUGGUAUUCAGUGUGAUGAut | |
| SB-0105 | F6 | ApoB | P | P3290 | Hex-uCAUCACACUGAAUACCAAut | 5′Click |
| | | | G | G3310 | IR700-uUGGUAUUCAGUGUGAUGAut | |
| SB-0106 | NAG21 | ApoB | P | P3312 | UCAUcACACUGAAUAcCAAut | Alkyne-Disulfide (ortho) |
| | | | G | G3314 | uUGGcAUUCAGUGUGaUGAut | |
| SB-0107 | NAG21 | ApoB | P | P3313 | UCAUcACAcUGaAUAcCAAut | Alkyne-Disulfide (ortho) |
| | | | G | G3292 | uUGGCAUUCAGUGUGAUGAut | |
| SB-0108 | NAG21 | ApoB | P | P3318 | Hex-UCAUcACACUGAAUAcCAAUt | 5′Click |
| | | | G | G3319 | UUGGuAUUCAGUGUGaUGAUt | |
| SB-0109 | NAG21 | ApoB | P | P3320 | Hex-UsCAUCACACUGAAUACCAAUst | 5′Click |
| | | | G | G3306 | UsUGGUAUUCAGUGUGAUGAUst | |
| SB-0110 | NAG21 | ApoB | P | P3295 | UCAUCACACUGAAUACCAAut | Alkyne-Disulfide (ortho) |
| | | | G | G3315 | uUGGuAUUcAGuGUGaUGAut | |
| SB-0111 | NAG21 | ApoB | P | P3295 | UCAUCACACUGAAUACCAAut | Alkyne-Disulfide (ortho) |
| | P34 | | G | G3303 | UUGGUAUUCAGUGUGAUGAut | Aldehyde-Disulfide (ortho) |
| SB-0112 | NAG21 | ApoB | P | P3295 | UCAUCACACUGAAUACCAAut | Alkyne-Disulfide (ortho) |
| | P32 | | G | G3303 | UUGGUAUUCAGUGUGAUGAut | Aldehyde-Disulfide (ortho) |
| SB-0113 | NAG21 | ApoB | P | P3295 | UCAUCACACUGAAUACCAAut | Alkyne-Disulfide (ortho) |
| | P17 | | G | G3303 | UUGGUAUUCAGUGUGAUGAut | Aldehyde-Disulfide (ortho) |
| SB-0114 | NAG21 | ApoB | P | P3295 | UCAUCACACUGAAUACCAAut | Alkyne-Disulfide (ortho) |
| | P18 | | G | G3303 | UUGGUAUUCAGUGUGAUGAut | Aldehyde-Disulfide (ortho) |
| SB-0115 | NAG21 | ApoB | P | P3295 | UCAUCACACUGAAUACCAAut | Alkyne-Disulfide (ortho) |
| | P33 | | G | G3303 | UUGGUAUUCAGUGUGAUGAut | Aldehyde-Disulfide (ortho) |
| SB-0116 | NAG21 | ApoB | P | P3295 | UCAUCACACUGAAUACCAAut | Alkyne-Disulfide (ortho) |
| | P35 | | G | G3303 | UUGGUAUUCAGUGUGAUGAut | Aldehyde-Disulfide (ortho) |
| SB-0117 | NAG21 | ApoB | P | P3295 | UCAUCACACUGAAUACCAAut | Alkyne-Disulfide (ortho) |
| | P05 | | G | G3303 | UUGGUAUUCAGUGUGAUGAut | Aldehyde-Disulfide (ortho) |
| SB-0118 | NAG21 | ApoB | P | P3295 | UCAUCACACUGAAUACCAAut | Alkyne-Disulfide (ortho) |
| | P04 | | G | G3303 | UUGGUAUUCAGUGUGAUGAut | Aldehyde-Disulfide (ortho) |
| SB-0119 | NAG21 | ApoB | P | P3295 | UCAUCACACUGAAUACCAAut | Alkyne-Disulfide (ortho) |
| | P20 | | G | G3303 | UUGGUAUUCAGUGUGAUGAut | Aldehyde-Disulfide (ortho) |
| SB-0120 | NAG21 | Factor | P | P3316 | AGGAUCAUCUCAAGUCUUAut | Alkyne-Disulfide (ortho) |
| | | VII | G | G3317 | uAAGACUUGAGAUGAUCCUut | |
| SB-0121 | NAG21 | ApoB | P | P3363 | Hex-UCAUCACACUGAAUACCAAUt | 5′Click |
| | | | G | G3366 | UUGGUAUUCAGUGUGAUGAUt | |
| SB-0122 | NAG21 | ApoB | P | P3359 | Hex-uCAUCACACUGAAUACCAAut | 5′Click |
| | | | G | G3360 | uUGGUAUUCAGUGUGAUGAut | |
| SB-0123 | NAG21 | ApoB | P | P3361 | [symbol] CAUCACACUGAAUACCAA [symbol] t | Alkyne-Disulfide (ortho) |
| | | | G | G3362 | [symbol] UGGUAUUCAGUGUGAUGA [symbol] t | |
| SB-0124 | M9 | GAPDH | P | | UCUACAUGUUCCAGUAUGAut | Alkyne-Disulfide (ortho) |
| | | | G | | uCAUACUGGAACAUGUAGAut | |
| SB-0130 | NAG21 | ApoB | P | P3373 | uCAUCACACUGAAUACCAAut | Alkyne-Disulfide (ortho) |
| | | | G | G3372 | UUGGUAUUCAGUGUGAUGAut | |
| SB-0132 | M9 | ApoB | P | | UCAUCACACUGAAUACCAAut | Alkyne-Disulfide (ortho) |
| | | | G | | uUGGUAUUCAGUGUGAUGAut | |
| SB-0133 | NAG21 | ApoB | P | P3295 | UCAUCACACUGAAUACCAAut | Alkyne-Disulfide (ortho) |
| | | | G | G3378 | uUGGUAUUCAGUGUGAUGAUst | |
| SB-0134 | NAG21 | ApoB | P | P3376 | UCAUCACACUGAAUACCAAUst | Alkyne-Disulfide (ortho) |
| | | | G | G3378 | uUGGUAUUCAGUGUGAUGAUst | |
| SB-0135 | NAG21 | ApoB | P | P3376 | UCAUCACACUGAAUACCAAUst | Alkyne-Disulfide (ortho) |
| | | | G | G3379 | UsUGGUAUUCAGUGUGAUGAut | |
| SB-0136 | NAG21 | ApoB | P | P3376 | UCAUCACACUGAAUACCAAUst | Alkyne-Disulfide (ortho) |
| | | | G | G3306 | UsUGGUAUUCAGUGUGAUGAUst | |
| SB-0137 | NAG21 | ApoB | P | P3377 | UCAUCACACUGAAUACCAAut | Alkyne-Disulfide (ortho) |
| | | | G | G3380 | uUGGUAUUCAGUGUGAUGAut | |
| SB-0138 | M9 | GAPDH | P | P3382 | Hex-UsCUACAUGUUCCAGUAUGAUst | 5′Click |
| | | | G | G3383 | UsCAUACUGGAACAUGUAGAUst | |
| SB-0139 | M9 | GAPDH | P | P3384 | Hex-uCUACAUGUUCCAGUAUGAut | 5′Click |
| | | | G | G3365 | uCAUACUGGAACAUGUAGAut | |
| SB-0140 | NAG21 | ApoB | P | P3454 | Hex-UsCAUCACACUGAAUACCAAUst | 5′Click |
| | | | G | G3457 | UsUGGUAUUCAGUGUGAUGAUst | |
| SB-0141 | P45 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0142 | NAG21 | ApoB | P | P3376 | UCAUCACACUGAAUACCAAUst | Alkyne-Disulfide (ortho) |
| | P45 | | G | G3381 | UUGGUAUUCAGUGUGAUGAUst | Alkyne-Disulfide (ortho) |
| SB-0146 | NAG21 | GAPDH | P | P3364 | UCUACAUGUUCCAGUAUGAut | Alkyne-Disulfide (ortho) |
| | | | G | G3365 | uCAUACUGGAACAUGUAGAut | |
| SB-0154 | NAG21 | ApoB | P | P3458 | Hex-UiCAUCACACUGAAUACCAAUit | 5′Click |
| | | | G | G3459 | UiUGGUAUUCAGUGUGAUGUit | |
| SB-0155 | NAG21 | ApoB | P | P3460 | Hex-UpCAUCACACUGAAUACCAAUpt | 5′Click |
| | | | G | G3461 | UpUGGUAUUCAGUGUGAUGUpt | |
| SB-0156 | NAG21 | ApoB | P | P3452 | Hex-UmCAUCACACUGAAUACCAAUmt | 5′Click |
| | | | G | G3453 | UmUGGUAUUCAGUGUGAUGUmt | |
| SB-0157 | NAG21 | ApoB | P | P3462 | Hex-UbCAUCACACUGAAUACCAAUbt | 5′Click |
| | | | G | G3463 | UbUGGUAUUCAGUGUGAUGUbt | |
| SB-0162 | NAG21 | ApoB | P | P3307 | uCAUCACACUGAAUACCAAut | |
| | | | G | G3372 | UUGGUAUUCAGUGUGAUGAut | Alkyne-Disulfide (ortho) |
| SB-0163 | P42 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0164 | P50 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0222 | NAG21 | ApoB | P | P3522 | UCAUCACACUGAAUACCAAut | Alkyne-Disulfide (ortho) |
| | | | G | G3292 | uUGGUAUUCAGUGUGAUGAut | |
| SB-0223 | NAG21 | ApoB | P | P3523 | UCAUCACACUGAAUACCAAut | Alkyne-Disulfide (ortho) |
| | | | G | G3292 | uUGGUAUUCAGUGUGAUGAut | |
| SB-0224 | NAG21 | ApoB | P | P3524 | UCAUCACACUGAAUACCAAut | Alkyne-Disulfide (ortho) |
| | | | G | G3292 | uUGGUAUUCAGUGUGAUGAut | |
| SB-0225 | NAG21 | ApoB | P | P3295 | UCAUCACACUGAAUACCAAut | Alkyne-Disulfide (ortho) |
| | NAG21 | | G | G3525 | uUGGUAUUCAGUGUGAUGAut | Alkyne-Disulfide (ortho) |
| SB-0226 | NAG21 | ApoB | P | P3523 | UCAUCACACUGAAUACCAAut | Alkyne-Disulfide (ortho) |
| | NAG21 | | G | G3525 | uUGGUAUUCAGUGUGAUGAut | Alkyne-Disulfide (ortho) |
| SB-0227 | M30 | GAPDH | P | P3364 | UCUACAUGUUCCAGUAUGAut | Alkyne-Disulfide (ortho) |
| | | | G | G3365 | uCAUACUGGAACAUGUAGAut | |
| SB-0228 | M30 | GAPDH | P | P3527 | UCUACAUGUUCCAGUAUGAut | Alkyne-Disulfide (ortho) |
| | | | G | G3365 | uCAUACUGGAACAUGUAGAut | |
| SB-0229 | M30 | GAPDH | P | P3528 | UCUACAUGUUCCAGUAUGAut | Alkyne-Disulfide (ortho) |
| | | | G | G3365 | uCAUACUGGAACAUGUAGAut | |
| SB-0230 | M30 | GAPDH | P | P3529 | UCUACAUGUUCCAGUAUGAUt | Alkyne-Disulfide (ortho) |
| | | | G | G3365 | uCAUACUGGAACAUGUAGAut | |
| SB-0231 | M30 | GAPDH | P | P3530 | UCUACAUGUUCCAGUAUGAut | Alkyne-Disulfide (ortho) |
| | | | G | G3365 | uCAUACUGGAACAUGUAGAut | |
| SB-0232 | M30 | GAPDH | P | P3528 | UCUACAUGUUCCAGUAUGAut | Alkyne-Disulfide (ortho) |
| | | | G | G3365 | uCAUACUGGAACAUGUAGAut | |
| SB-0233 | PSMA10 | GAPDH | P | P3526 | UCUACAUGUUCCAGUAUGAut | Aldehyde-Disulfide (ortho) |
| | | | G | G3365 | uCAUACUGGAACAUGUAGAut | |
| SB-0234 | P51 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0235 | P52 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0236 | P49 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0237 | P37 | ApoB | P | P3307 | uCAUCACACUGAAUACCAAut | Alkyne-Disulfide (ortho) |
| SB-0238 | P38 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0239 | P39 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0240 | P47 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0241 | P46 | ApoB | P | P3307 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0242 | M15 | GAPDH | P | P3364 | UCUACAUGUUCCAGUAUGAut | Alkyne-Disulfide (ortho) |
| | | | G | G3365 | uCAUACUGGAACAUGUAGAut | |
| SB-0248 | M15 | GAPDH | P | P3527 | UCUACAUGUUCCAGUAUGAut | Alkyne-Disulfide (ortho) |
| | | | G | G3365 | uCAUACUGGAACAUGUAGAut | |
| SB-0249 | M15 | GAPDH | P | P3528 | UCUACAUGUUCCAGUAUGAut | Alkyne-Disulfide (ortho) |
| | | | G | G3365 | uCAUACUGGAACAUGUAGAut | |
| SB-0250 | M15 | GAPDH | P | P3529 | UCUACAUGUUCCAGUAUGAUt | Alkyne-Disulfide (ortho) |
| | | | G | G3365 | uCAUACUGGAACAUGUAGAut | |
| SB-0251 | M15 | GAPDH | P | P3530 | UCUACAUGUUCCAGUAUGAut | Alkyne-Disulfide (ortho) |
| | | | G | G3365 | uCAUACUGGAACAUGUAGAut | |
| SB-0252 | M15 | GAPDH | P | P3528 | UCUACAUGUUCCAGUAUGAut | Alkyne-Disulfide (ortho) |
| | | | G | G3365 | uCAUACUGGAACAUGUAGAut | |
| SB-0253 | PSMA20 | GAPDH | P | P3526 | UCUACAUGUUCCAGUAUGAut | Aldehyde-Disulfide (ortho) |
| | | | G | G3365 | uCAUACUGGAACAUGUAGAut | |
| SB-0259 | P45 | ApoB | P | P3551 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0260 | P45 | ApoB | P | P3551 | uCAUCACACUGAAUACCAAut | Alkyne-Disulfide (ortho) |
| | NAG21 | | G | G3372 | UUGGUAUUCAGUGUGAUGAut | Alkyne-Disulfide (ortho) |
| SB-0285 | NAG21 | ApoB | P | P3553 | Hex-U(m1)CAUCACACUGAAUACCAAU(m1)t | 5 Click |
| | | | G | G3554 | U(m1)UGGUAUUCAGUGUGAUGU(m1)t | |
| SB-0286 | NAG21 | ApoB | P | P3555 | Hex-U(m2)CAUCACACUGAAUACCAAU(m2)t | 5′Click |
| | | | G | G3556 | U(m2)UGGUAUUCAGUGUGAUGU(m2)t | |

The mixed siRNA conjugates of the invention are provided in Table 6:

TABLE 6

| Compound # | Ligand | Target | Strand | Strand # | Sequences (5′ - 3′) | Conjugation-Prodrug Unker |
|---|---|---|---|---|---|---|
| SB-0097 | NAG21 | ApoB | P | P3295 | UCAUCACACUGAAUACCAAut | Alkyne-Disulfide (ortho) |
| | | | G | G3292 | uUGGUAUUCAGUGUGAUGAut | N/A |
| SB-0111 | NAG21 | ApoB | P | P3295 | UCAUCACACUGAAUACCAAut | Alkyne-Disulfide (ortho) |
| | P34 | | G | G3303 | UUGGUAUUCAGUGUGAUGAut | Aldehyde-Disulfide(ortho) |
| SB-0112 | NAG21 | ApoB | P | P3295 | UCAUCACACUGAAUACCAAut | Alkyne-Disulfide (ortho) |
| | P32 | | G | G3303 | UUGGUAUUCAGUGUGAUGAut | Aldehyde-Disulfide(ortho) |
| SB-0113 | NAG21 | ApoB | P | P3295 | UCAUCACACUGAAUACCAAut | Alkyne-Disulfide (ortho) |
| | P17 | | G | G3303 | UUGGUAUUCAGUGUGAUGAut | Aldehyde-Disulfide(ortho) |
| SB-0114 | NAG21 | ApoB | P | P3295 | UCAUCACACUGAAUACCAAut | Alkyne-Disulfide (ortho) |
| | P18 | | G | G3303 | UUGGUAUUCAGUGUGAUGAut | Aldehyde-Disulfide(ortho) |
| SB-0115 | NAG21 | ApoB | P | P3295 | UCAUCACACUGAAUACCAAut | Alkyne-Disulfide (ortho) |
| | P33 | | G | G3303 | UUGGUAUUCAGUGUGAUGAut | Aldehyde-Disulfide(ortho) |
| SB-0116 | NAG21 | ApoB | P | P3295 | UCAUCACACUGAAUACCAAut | Alkyne-Disulfide (ortho) |
| | P35 | | G | G3303 | UUGGUAUUCAGUGUGAUGAut | Aldehyde-Disulfide(ortho) |
| SB-0117 | NAG21 | ApoB | P | P3295 | UCAUCACACUGAAUACCAAut | Alkyne-Disulfide (ortho) |
| | P05 | | G | G3303 | UUGGUAUUCAGUGUGAUGAut | Aldehyde-Disulfide(ortho) |
| SB-0118 | NAG21 | ApoB | P | P3295 | UCAUCACACUGAAUACCAAut | Alkyne-Disulfide (ortho) |
| | P04 | | G | G3303 | UUGGUAUUCAGUGUGAUGAut | Aldehyde-Disulfide(ortho) |
| SB-0119 | NAG21 | ApoB | P | P3295 | UCAUCACACUGAAUACCAAut | Alkyne-Disulfide (ortho) |
| | P20 | | G | G3303 | UUGGUAUUCAGUGUGAUGAut | Aldehyde-Disulfide(ortho) |

For Tables 5 and 6: UPPER CASE=2′OMe Purines, 2′F Pyrimidines; lower case=deoxy; lower case bold=3,3-dimethylbutyl (DMB); BOLD=iPrDS (ortho) >(2′OMe Purines, 2′F Pyrimidines); ITALICS=tBuDS (2′OMe Purines, 2′F Pyrimidines); lowercase italics=tBuDS-Ph (ortho) (2′OMe Purines, 2′F Pyrimidines); 0 BOLD ITALICS=tBuDS-Ph (ortho)-Phosphorothioate (2′OMe Purines, 2′F Pyrimidines); UNDERLINE=Conjugated Prodrug Location; s=Phosphorothioate; z m i=NMI-DS-Ph; p=PEG4-DS-Ph; m=tBuDS-Ph(Me); b=tBuDS-Ph(Br); ml=tBuDS-(ml)Me-Ph; m2=tBuDS-(m2)Me-Ph; Ald=5′ Benzaldehyde; Hex=5′ Hexynyl; o IR=infrared imaging dye; Cy3 is cyanine Cy3 dye; DS means disulfide; for the purposed of this table, Ph means phenethyl.

TABLE 7

| Compound | | Tar- | | Strand | | Conjugation |
|---|---|---|---|---|---|---|
| # | Ligand | get | Strand | # | Sequences (5′ - 3′) | Unker |
| SB-0129 | NAG21 | ApoB | P | P3371 | UpCaUCaCaCUgaaUaCCaaUt | Propargyl |
| | | | G | G3290 | UUggUaUUCagUgUgaUgaUt | |
| SB-0158 | None | GAPDH | P | P3364 | UCUaCaUgUUCCagUaUgaUt | None |
| | | | G | G3365 | UCaUaCUggaaCaUgUagaUt | |
| SB-0206 | NAG21 | AT3 | P | P3507 | gpgUuAaCaCCAuUuAcUuCaa | Propargyl |
| | | | G | G3508 | UUgAaGuAaAuggUgUuAaCcag | |
| SB-0209 | NAG21 | AT3 | P | P3503 | GsgsUuAaCaCCAuUuAcUuCaA-Alk | 3′Alkyne |
| | | | G | G3504 | usUsgAaGuAaAuggUgUuAaCcsasg | |
| SB-0210 | NAG21 | AT3 | P | P3503 | gsgsUuAaCaCCAuUuAcUuCaa-Alk | 3′Alkyne |
| | | | G | G3504 | UsUsgAaGuAaAuggUgUuAaCcsasg | |
| SB-0211 | NAG21 | AT3 | P | P3511 | Hex-gsgsUuAaCaCCAuUuAcUuCaa | 5′Hexynyl |
| | | | G | G3504 | UsUsgAaGuAaAuggUgUuAaCcsasg | |
| SB-0212 | NAG21 | AT3 | P | P3512 | ggUuAaCaCCAuUuAcUuCaa-Alk | 3′Alkyne |
| | | | G | G3508 | UUgAaGuAaAuggUgUuAaCcag | |
| SB-0213 | NAG21 | AT3 | P | P3513 | Hex-ggUuAaCaCCAuUuAcUuCaa | 5′Hexynyl |
| | | | G | G3508 | UUgAaGuAaAuggUgUuAaCcag | |
| SB-0243 | NAG21 | ApoB | P | P3371 | UpCaUCaCaCUgaaUaCCaaUt | Propargyl |
| | | | G | G3487 | UUggUpaUUCagUgUgaUgaUt | |
| SB-0244 | NAG21 | ApoB | P | P3371 | UpCaUCaCaCUgaaUaCCaaUt | Propargyl |
| | | | G | G3496 | UUggUaUUCagUgUpgaUgaUt | |
| SB-0245 | NAG21 | ApoB | P | P3373 | UCaUCaCaCUgaaUaCCaaUt | |
| | | | G | G3487 | UUggUpaUUCagUgUgaUgaUt | Propargyl |
| SB-0246 | NAG21 | ApoB | P | P3373 | UCaUCaCaCUgaaUaCCaaUt | |
| | | | G | G3496 | UUggUaUUCagUgUpgaUgaUt | Propargyl |
| SB-0254 | NAG21 | AT3 | P | P3532 | Hex-ggUuAaCaCCAuUuAcUuCaa | 5′Hexynyl |
| | | | G | G3533 | UUgAaGuAaAuggUgUuAaCcag | |
| SB-0256 | NAG21 | AT3 | P | P3507 | gpgUuAaCaCCAuUuAcUuCaa | Propargyl |
| | | | G | G3548 | UUgAapGuAaAuggUgUuAaCcag | |
| SB-0257 | NAG21 | AT3 | P | P3507 | gpgUuAaCaCCAuUuAcUuCaa | Propargyl |
| | | | G | G3549 | UUgAaGuAaAuggUgUupAaCcag | |
| SB-0258 | NAG21 | AT3 | P | P3507 | gpgUuAaCaCCAuUuAcUuCaa | Propargyl |
| | | | G | G3550 | UUgAapGuAaAuggUgUupAaCcag | |
| SB-0274 | NAG21 | ApoB | P | P3371 | UpCaUCaCaCUgaaUaCCaaUt | Propargyl |
| | | | G | G3535 | UUggUpaUUCagUgUgaUpgaUt | |
| SB-0275 | NAG21 | ApoB | P | P3371 | UpCaUCaCaCUgaaUaCCaaUt | Propargyl |
| | | | G | G3536 | UUggUdaUUCagUgUgaUgaUt | |
| SB-0276 | NAG21 | ApoB | P | P3371 | UpCaUCaCaCUgaaUaCCaaUt | Propargyl |
| | | | G | G3539 | UUggUaUUCagUgUgaUgaUt | |
| SB-0277 | NAG21 | ApoB | P | P3371 | UpCaUCaCaCUgaaUaCCaaUt | Propargyl |
| | | | G | G3540 | UUggUaUUCagUgUgaUgaUt | |
| SB-0278 | NAG21 | ApoB | P | P3371 | UpCaUCaCaCUgaaUaCCaaUt | Propargyl |
| | | | G | G3541 | UUggUaUUCagUgUgaUgaUt | |
| SB-0279 | NAG21 | ApoB | P | P3371 | UpCaUCaCaCUgaaUaCCaaUt | Propargyl |
| | | | G | G3542 | UUggUbaUUCagUgUgaUgaUt | |
| SB-0280 | NAG21 | ApoB | P | P3371 | UpCaUCaCaCUgaaUaCCaaUt | Propargyl |
| | | | G | G3543 | UUggUaUUCagUgUgaUbgaUt | |
| SB-0281 | NAG21 | ApoB | P | P3371 | UpCaUCaCaCUgaaUaCCaaUt | Propargyl |
| | | | G | G3544 | UUggUbaUUCagUgUgaUbgaUt | |
| SB-0282 | NAG21 | ApoB | P | P3371 | UpCaUCaCaCUgaaUaCCaaUt | Propargyl |
| | | | G | G3545 | UUggUPaUUCagUgUgaUgaUt | |
| SB-0283 | NAG21 | ApoB | P | P3371 | UpCaUCaCaCUgaaUaCCaaUt | Propargyl |
| | | | G | G3546 | UUggUaUUCagUgUgaUPgaUt | |
| SB-0284 | NAG21 | ApoB | P | P3371 | UpCaUCaCaCUgaaUaCCaaUt | Propargyl |
| | | | G | G3547 | UUggUPaUUCagUgUgaUPgaUt | |
| SB-0289 | NAG21 | ApoB | P | P3557 | UpCaUCaCaCUgaaUaCCaaUbt | Propargyl |
| | | | G | G3558 | UUggUaUUCagUgUgaUgaUpt | |
| SB-0290 | NAG21 | ApoB | P | P3557 | UpCaUCaCaCUgaaUaCCaaUbt | Propargyl |
| | | | G | G3559 | UpUggUaUUCagUgUgaUgaUpt | |
| SB-0291 | NAG21 | ApoB | P | P3560 | UpCbaUCaCaCUgaaUaCCaaUbt | Propargyl |
| | | | G | G3558 | UUggUaUUCagUgUgaUgaUpt | |
| SB-0292 | NAG21 | ApoB | P | P3557 | UpCaUCaCaCUgaaUaCCaaUbt | Propargyl |
| | | | G | G3561 | UUpggUaUUCagUgUgaUgaUpt | |
| SB-0293 | NAG21 | ApoB | P | P3560 | UpCbaUCaCaCUgaaUaCCaaUbt | Propargyl |
| | | | G | G3561 | UUpggUaUUCagUgUgaUgaUpt | |
| SB-0294 | NAG21 | ApoB | P | P3560 | UpCbaUCaCaCUgaaUaCCaaUbt | Propargyl |
| | | | G | G3562 | UUbggUaUUCagUgUgaUgaUbt | |
| SB-0295 | NAG21 | AT3 | P | P3563 | gpgUbuAaCaCCAuUuAcUuCaa | Propargyl |
| | | | G | G3564 | UUbgAaGuAaAuggUgUuAaCcag | |
| SB-0296 | NAG21 | AT3 | P | P3563 | gpgUbuAaCaCCAuUuAcUuCaa | Propargyl |
| | | | G | G3508 | UUgAaGuAaAuggUgUuAaCcag | |
| SB-0297 | NAG21 | AT3 | P | P3507 | gpgUuAaCaCCAuUuAcUuCaa | Propargyl |
| | | | G | G3564 | UUbgAaGuAaAuggUgUuAaCcag | |
| SB-0298 | NAG21 | ApoB | P | P3565 | UpcAuCaCaCuGaAuAcCaAUt | Propargyl |
| | | | G | G3566 | UUgGuAuUcAgUgUgAuGaUt | |
| SB-0299 | NAG21 | ApoB | P | P3567 | UpcAuCaCaCuGaAuAcCaAUg | Propargyl |
| | | | G | G3568 | UUgGuAuUcAgUgUgAuGaCa | |
| SB-0300 | NAG21 | ApoB | P | P3569 | UpgUcAuCaCACuGaAuAcCaA | Propargyl |
| | | | G | G3570 | UUgGuAuUcAgugUgAuGaCaCu | |
| SB-0310 | M9 | Luc | P | 3571 | gpCUaCaUUCUggagaCaUaUt | Propargyl |
| | | | G | 3572 | UaUgUCUCCagaaUgUagCUt | |
| SB-0311 | None | Luc | P | 3571 | gpCUaCaUUCUggagaCaUaUt | None |
| | | | G | 3572 | UaUgUCUCCagaaUgUagCUt | |
| SB-0312 | None | Luc | P | 3024 | gCUaCaUUCUggagaCaUaUt | None |
| | | | G | 3025 | UaUgUCUCCagaaUgUagCUt | |
| SB-0313 | None | GAPDH | P | P3101 | UCUaCaUgUUCCagUaUgaUt | None |
| | | | G | G3102 | UCaUaCUggaaCaUgUagaUt | |
| SB-0314 | NAG21 | ApoB | P | P3574 | UpaCaUCaCaCUgaaUaCCaaUPt | Propargyl |
| | | | G | G3575 | UUggUaUUCagUgUgaUgaUPt | |
| SB-0315 | NAG21 | ApoB | P | P3574 | UpCaUCaCaCUgaaUaCCaaUPt | Propargyl |
| | | | G | G3576 | UPUggUaUUCagUgUgaUgaUPt | |
| SB-0316 | NAG21 | ApoB | P | P3574 | UpCaUCaCaCUgaaUaCCaaUPt | Propargyl |
| | | | G | G3577 | UUggUaUUCagUgUgaUgaUPt | |
| SB-0317 | NAG21 | ApoB | P | P3371 | UpCaUCaCaCUgaaUaCCaaUt | Propargyl |
| | | | G | G3578 | UUggUaUUCagUgUgaUgaUt | |
| SB-0318 | NAG21 | ApoB | P | P3371 | UpCaUCaCaCUgaaUaCCaaUt | Propargyl |
| | | | G | G3579 | UUggUaUUCagUgUgaUgaUt | |
| SB-0319 | NAG21 | AT3 | P | P3507 | gpgUuAaCaCCAuUuAcUuCaa | Propargyl |
| | | | G | G3580 | UUgAaGuAaAuggUgUuAaCcag | |
| SB-0320 | NAG21 | AT3 | P | P3507 | gpgUuAaCaCCAuUuAcUuCaa | Propargyl |
| | | | G | G3581 | UUgAaGuAaAuggUgUuAaCCag | |
| SB-0321 | NAG21 | AT3 | P | P3582 | gpgUuAaCaCCAuUuAcUuCaba | Propargyl |
| | | | G | G3583 | UUgAaGuAaAuggUgUuAaCcabg | |
| SB-0322 | NAG21 | AT3 | P | P3582 | gpgUuAaCaCCAuUuAcUuCaba | Propargyl |
| | | | G | G3584 | UUgAaGuAaAuggUgUuAaCcabg | |
| SB-0323 | NAG21 | AT3 | P | P3582 | gpgUuAaCaCCAuUuAcUuCaba | Propargyl |
| | | | G | G3585 | UUgAaGuAaAuggUgUuAaCCabg | |
| SB-0324 | M9 | GAPDH | P | P3586 | UpCUaCaUgUUCCagUaUgaUt | Propargyl |
| | | | G | G3365 | UCaUaCUggaaCaUgUagaUt | |
| SB-0325 | M9 | GAPDH | P | P3587 | UpCUaCaUgUUCCagUaUgaUit | Propargyl |
| | | | G | G3589 | UiCaUaCUggaaCaUgUagaUit | |
| SB-0326 | M9 | GAPDH | P | P3588 | UpCUaCaUgUUCCagUaUgaUpt | Propargyl |
| | | | G | G3590 | UpCaUaCUggaaCaUgUagaUpt | |
| SB-0327 | None | GAPDH | P | P3586 | UpCUaCaUgUUCCagUaUgaUt | None |
| | | | G | G3365 | UCaUaCUggaaCaUgUagaUt | |
| SB-0328 | None | GAPDH | P | P3587 | UpCUaCaUgUUCCagUaUgaUit | None |
| | | | G | G3589 | UiCaUaCUggaaCaUgUagaUit | |
| SB-0329 | None | GAPDH | P | P3588 | UpCUaCaUgUUCCagUaUgaUpt | None |
| | | | G | G3590 | UpCaUaCUggaaCaUgUagaUpt | |
| SB-0330 | None | AT3 | P | P3507 | gpgUuAaCaCCAuUuAcUuCaa | None |
| | | | G | G3508 | UUgAaGuAaAuggUgUuAaCcag | |
| SB-0332 | NAG21 | AT3 | P | P3582 | gpgUuAaCaCCAuUuAcUuCaba | Propargyl |
| | | | G | G3508 | UUgAaGuAaAuggUgUuAaCcag | |
| SB-0333 | NAG21 | AT3 | P | P3591 | gbgUAaCaCCAuUuAcUuCapa | Propargyl |
| | | | G | G3508 | UUgAaGuAaAuggUgUuAaCcag | |
| SB-0334 | NAG21 | AT3 | P | P3592 | gpgUPuAaCaCCAuUuAcUuCAa | Propargyl |
| | | | G | G3508 | UUgAaGuAaAuggUgUuAaCcag | |
| SB-0335 | NAG21 | AT3 | P | P3593 | gpgUbuabaCaCCAuUuAcUuCAa | Propargyl |
| | | | G | G3508 | UUgAaGuAaAuggUgUuAaCcag | |
| SB-0336 | NAG21 | AT3 | P | P3594 | gpgUbuabaCbaCCAuUuAcUuCAa | Propargyl |
| | | | G | G3508 | UUgAaGuAaAuggUgUuAaCcag | |
| SB-0337 | NAG21 | AT3 | P | P3595 | gbgUuAaCaCCAuUUpAcUuCAa | |
| | | | G | G3596 | UUgAaGuAaAuggUgUuAaCcag | AlkDS-Ph |
| SB-0338 | ABL5 | AT3 | P | P3595 | gbgUAaCaCCAuUUpAcUuCAa | Propargyl |
| | NAG21 | | G | G3596 | UUgAaGuAaAuggUgUuAaCcag | AlkDS-Ph |
| SB-0339 | NAG21 | AT3 | P | P3600 | gpgUUaaCaCCaUUUaCUUCaa | Propargyl |
| | | | G | G3601 | UUgaagUaaaUggUgUUaaCCag | |
| SB-0340 | NAG21 | AT3 | P | P3602 | gpgUuAaCaCCAuUuAcUuCaagbg | Propargyl |
| | | | G | G3508 | UUgAaGuAaAuggUgUuAaCcag | |
| SB-0341 | NAG21 | AT3 | P | P3507 | gpgUuAaCaCCAuUuAcUuCaa | Propargyl |
| | | | G | G3603 | UUgAaGuAaAuggUgUuAaCcUt | |
| SB-0342 | NAG21 | AT3 | P | P3604 | gpgUuAaCaCCAuUuAcUuCaaUt | Propargyl |
| | | | G | G3603 | UUgAaGuAaAuggUgUuAaCcUt | |
| SB-0343 | NAG21 | AT3 | P | P3605 | UpuAaCaCCAuUuAcUuCaagbg | Propargyl |
| | | | G | G3606 | UUgAaGuAaAuggUgUuAaCc | |
| SB-0344 | NAG21 | AT3 | P | P3607 | UpuAaCaCCAuUuAcUuCaaUt | Propargyl |
| | | | G | G3608 | UUgAaGuAaAuggUgUuAaUt | |
| SB-0345 | NAG21 | AT3 | P | P3598 | gbgUmpuAaCaCCAuUuAcUuCaa | Methyl-Propargyl |
| | | | G | G3508 | UUgAaGuAaAuggUgUuAaCcag | |
| SB-0346 | NAG21 | AT3 | P | P3599 | gbgUpuAaCaCCAuUuAcUuCaa | Propargyl |
| | | | G | G3508 | UUgAaGuAaAuggUgUuAaCcag | |
| SB-0347 | SP1L | AT3 | P | P3595 | gbgUAaCaCCAuUUpAcUuCaa | Propargyl |
| | NAG21 | | G | G3596 | UUgAaGuAaAuggUgUuAaCcag | AlkDS-Ph |
| SB-0348 | NAG21 | AT3 | P | P3507 | gpgUuAaCaCCAuUuAcUuCaa | Propargyl |
| | | | G | G3615 | UpUgAaGuAaAuggUgUuAaCcapg | |
| SB-0349 | NAG21 | AT3 | P | P3507 | gpgUuAaCaCCAuUuAcUuCaa | Propargyl |
| | | | G | G3616 | Ph-UpUgAaGuAaAuggUgUuAaCcapg | |
| SB-0351 | PEG24 | GAPDH | P | P3620 | UpCUpaCaUgUUCCagUaUpgaUpt | Propargyl |
| | | | G | G3102 | UCaUaCUggaaCaUgUagaUt | |
| SB-0353 | PEG4 | GAPDH | P | P3619 | UpCUaCaUgUUCCagUaUgaUpt | Propargyl |
| | | | G | G3102 | UCaUaCUggaaCaUgUagaUt | |
| SB-0354 | PEG24 | GAPDH | P | P3619 | UpCUaCaUgUUCCagUaUgaUpt | Propargyl |
| | | | G | G3102 | UCaUaCUggaaCaUgUagaUt | |
| SB-0355 | PEG4 | GAPDH | P | P3620 | UpCUpaCaUgUUCCagUaUpgaUpt | Propargyl |
| | | | G | G3102 | UCaUaCUggaaCaUgUagaUt | |
| SB-0356 | NAG28 | GAPDH | P | P3364 | UCUaCaUgUUCCagUaUgaUt | AlkDS-Ph |
| | | | G | G3365 | UCaUaCUggaaCaUgUagaUt | |
| SB-0357 | NAG28 | GAPDH | P | P3527 | UCUaCaUgUUCCagUaUgaUt | AlkDS-Ph |
| | | | G | G3365 | UCaUaCUggaaCaUgUagaUt | |
| SB-0358 | NAG28 | GAPDH | P | P3528 | UCUaCaUgUUCCagUaUgaUt | AlkDS-Ph |
| | | | G | G3365 | UCaUaCUggaaCaUgUagaUt | |
| SB-0359 | NAG28 | GAPDH | P | P3529 | UCUaCaUgUUCCagUaUgaUt | AlkDS-Ph |
| | | | G | G3365 | UCaUaCUggaaCaUgUagaUt | |
| SB-0360 | NAG28 | GAPDH | P | P3530 | UCUaCaUgUUCCagUaUgaUt | AlkDS-Ph |
| | | | G | G3365 | UCaUaCUggaaCaUgUagaUt | |
| SB-0366 | NAG21 | AT3 | P | P3611 | gpgUuAaCaCCAuUuAcUuCapa | Propargyl |
| | | | G | G3615 | UpUgAaGuAaAuggUgUuAaCcapg | |
| SB-0367 | NAG21 | AT3 | P | P3612 | gpgUhuAaCaCCAuUuAcUuCapa | Propargyl |
| | | | G | G3615 | UpUgAaGuAaAuggUgUuAaCcapg | |
| SB-0368 | NAG21 | AT3 | P | P3613 | gpgUuAaCaCCAuUhuAcUuCapa | Propargyl |
| | | | G | G3615 | UpUgAaGuAaAuggUgUuAaCcapg | |
| SB-0369 | NAG21 | AT3 | P | P3614 | gpgUuAaCaCCAuUuAcUhuCapa | Propargyl |
| | | | G | G3615 | UpUgAaGuAaAuggUgUuAaCcapg | |
| SB-0370 | None | AT3 | P | P3627 | gpgUuAaCaCCAuUuAcUuCapa | |
| | NAG21 | | G | G3630 | UUgAaGuAaAuggUgUuAaCcapg | AlkDS-Ph |
| SB-0371 | PEG4 | AT3 | P | P3627 | gpgUuAaCaCCAuUuAcUuCapa | Propargyl |
| | NAG21 | | G | G3630 | UUgAaGuAaAuggUgUuAaCcapg | AlkDS-Ph |
| SB-0372 | PEG8 | AT3 | P | P3627 | gpgUuAaCaCCAuUuAcUuCapa | Propargyl |
| | NAG21 | | G | G3630 | UUgAaGuAaAuggUgUuAaCcapg | AlkDS-Ph |
| SB-0373 | PEG12 | AT3 | P | P3627 | gpgUuAaCaCCAuUuAcUuCapa | Propargyl |
| | NAG21 | | G | G3630 | UUgAaGuAaAuggUgUuAaCcapg | AlkDS-Ph |
| SB-0374 | PEG24 | AT3 | P | P3627 | gpgUuAaCaCCAuUuAcUuCapa | Propargyl |
| | NAG21 | | G | G3630 | UUgAaGuAaAuggUgUuAaCcapg | AlkDS-Ph |
| SB-0375 | None | AT3 | P | P3628 | gpgUpuAaCaCCAuUuAcUpuCapa | |
| | NAG21 | | G | G3630 | UUgAaGuAaAuggUgUuAaCcapg | AlkDS-Ph |
| SB-0376 | PEG4 | AT3 | P | P3628 | gpgUpuAaCaCCAuUuAcUpuCapa | Propargyl |
| | NAG21 | | G | G3630 | UUgAaGuAaAuggUgUuAaCcapg | AlkDS-Ph |
| SB-0377 | PEG8 | AT3 | P | P3628 | gpgUpuAaCaCCAuUuAcUpuCapa | Propargyl |
| | NAG21 | | G | G3630 | UUgAaGuAaAuggUgUuAaCcapg | AlkDS-Ph |
| SB-0378 | PEG12 | AT3 | P | P3628 | gpgUpuAaCaCCAuUuAcUpuCapa | Propargyl |
| | NAG21 | | G | G3630 | UUgAaGuAaAuggUgUuAaCcapg | AlkDS-Ph |
| SB-0379 | PEG24 | AT3 | P | P3628 | gpgUpAaCaCCAuUuAcUpuCapa | Propargyl |
| | NAG21 | | G | G3630 | UUgAaGuAaAuggUgUuAaCcapg | AlkDS-Ph |
| SB-0381 | NAG21 | AT3 | P | P3609 | DBCO-gbgUuAaCaCCAuUuAcUuCaa | DBCO |
| | | | G | G3508 | UUgAaGuAaAuggUgUuAaCcag | |
| SB-0382 | NAG21 | AT3 | P | P3610 | Hex-gbgUuAaCaCCAuUuAcUuCaa | Hexynyl |
| | | | G | G3508 | UUgAaGuAaAuggUgUuAaCcag | |
| SB-0383 | GAPDH | | P | P3619 | UpCUaCaUgUUCCagUaUgaUpt | |
| | | | G | G3625 | UCaUaCUggaaCaUgUagaUpt | |
| SB-0384 | PEG4 | GAPDH | P | P3619 | UpCUaCaUgUUCCagUaUgaUpt | Propargyl |
| | | | G | G3625 | UCaUaCUggaaCaUgUagaUpt | |
| SB-0385 | PEG8 | GAPDH | P | P3619 | UpCUaCaUgUUCCagUaUgaUpt | Propargyl |
| | | | G | G3625 | UCaUaCUggaaCaUgUagaUpt | |
| SB-0386 | PEG12 | GAPDH | P | P3619 | UpCUaCaUgUUCCagUaUgaUpt | Propargyl |
| | | | G | G3625 | UCaUaCUggaaCaUgUagaUpt | |
| SB-0387 | PEG24 | GAPDH | P | P3619 | UpCUaCaUgUUCCagUaUgaUpt | Propargyl |
| | | | G | G3625 | UCaUaCUggaaCaUgUagaUpt | |
| SB-0388 | M9 | GAPDH | P | P3619 | UpCUaCaUgUUCCagUaUgaUpt | |
| | | | G | G3625 | UCaUaCUggaaCaUgUagaUpt | AlkDS-Ph |
| SB-0389 | PEG4 | GAPDH | P | P3619 | UpCUaCaUgUUCCagUaUgaUpt | Propargyl |
| | M9 | | G | G3625 | UCaUaCUggaaCaUgUagaUpt | AlkDS-Ph |
| SB-0390 | PEG8 | GAPDH | P | P3619 | UpCUaCaUgUUCCagUaUgaUpt | Propargyl |
| | M9 | | G | G3625 | UCaUaCUggaaCaUgUagaUpt | AlkDS-Ph |
| SB-0391 | PEG12 | GAPDH | P | P3619 | UpCUaCaUgUUCCagUaUgaUpt | Propargyl |
| | M9 | | G | G3625 | UCaUaCUggaaCaUgUagaUpt | AlkDS-Ph |
| SB-0392 | PEG24 | GAPDH | P | P3619 | UpCUaCaUgUUCCagUaUgaUpt | Propargyl |
| | M9 | | G | G3625 | UCaUaCUggaaCaUgUagaUpt | AlkDS-Ph |
| SB-0393 | | GAPDH | P | P3620 | UpCUpaCaUgUUCCagUaUpgaUpt | |
| | | | G | G3625 | UCaUaCUggaaCaUgUagaUpt | |
| SB-0394 | PEG4 | GAPDH | P | P3620 | UpCUpaCaUgUUCCagUaUpgaUpt | Propargyl |
| | | | G | G3625 | UCaUaCUggaaCaUgUagaUpt | |
| SB-0395 | PEG8 | GAPDH | P | P3620 | UpCUpaCaUgUUCCagUaUpgaUpt | Propargyl |
| | | | G | G3625 | UCaUaCUggaaCaUgUagaUpt | |
| SB-0396 | PEG12 | GAPDH | P | P3620 | UpCUpaCaUgUUCCagUaUpgaUpt | Propargyl |
| | | | G | G3625 | UCaUaCUggaaCaUgUagaUpt | |
| SB-0397 | PEG24 | GAPDH | P | P3620 | UpCUpaCaUgUUCCagUaUpgaUpt | Propargyl |
| | | | G | G3625 | UCaUaCUggaaCaUgUagaUpt | |
| SB-0398 | M9 | GAPDH | P | P3620 | UpCUpaCaUgUUCCagUaUpgaUpt | |
| | | | G | G3625 | UCaUaCUggaaCaUgUagaUpt | AlkDS-Ph |
| SB-0399 | PEG4 | GAPDH | P | P3620 | UpCUpaCaUgUUCCagUaUpgaUpt | Propargyl |
| | M9 | | G | G3625 | UCaUaCUggaaCaUgUagaUpt | AlkDS-Ph |
| SB-0400 | PEG8 | GAPDH | P | P3620 | UpCUpaCaUgUUCCagUaUpgaUpt | Propargyl |
| | M9 | | G | G3625 | UCaUaCUggaaCaUgUagaUpt | AlkDS-Ph |
| SB-0401 | PEG12 | GAPDH | P | P3620 | UpCUpaCaUgUUCCagUaUpgaUpt | Propargyl |
| | M9 | | G | G3625 | UCaUaCUggaaCaUgUagaUpt | AlkDS-Ph |
| SB-0402 | PEG24 | GAPDH | P | P3620 | UpCUpaCaUgUUCCagUaUpgaUpt | Propargyl |
| | M9 | | G | G3625 | UCaUaCUggaaCaUgUagaUpt | AlkDS-Ph |
| SB-0403 | | GAPDH | P | P3621 | UpCUpaCaUpgUUCCagUpaUpgaUpt | |
| | | | G | G3625 | UCaUaCUggaaCaUgUagaUpt | |
| SB-0404 | PEG4 | GAPDH | P | P3621 | UpCUpaCaUpgUUCCagUpaUpgaUpt | Propargyl |
| | | | G | G3625 | UCaUaCUggaaCaUgUagaUpt | |
| SB-0405 | PEG8 | GAPDH | P | P3621 | UpCUpaCaUpgUUCCagUpaUpgaUpt | Propargyl |
| | | | G | G3625 | UCaUaCUggaaCaUgUagaUpt | |
| SB-0406 | PEG12 | GAPDH | P | P3621 | UpCUpaCaUpgUUCCagUpaUpgaUpt | Propargyl |
| | | | G | G3625 | UCaUaCUggaaCaUgUagaUpt | |
| SB-0407 | PEG24 | GAPDH | P | P3621 | UpCUpaCaUpgUUCCagUpgaUpgaUpt | Propargyl |
| | | | G | G3625 | UCaUaCUggaaCaUgUagaUpt | |
| SB-0408 | M30 | GAPDH | P | P3621 | UpCUpaCaUpgUUCCagUpaUpgaUpt | |
| | | | G | G3625 | UCaUaCUggaaCaUgUagaUpt | AlkDS-Ph |
| SB-0409 | PEG4 | GAPDH | P | P3621 | UpCUpaCaUpgUUCCagUpaUpgaUpt | Propargyl |
| | M30 | | G | G3625 | UCaUaCUggaaCaUgUagaUpt | AlkDS-Ph |
| SB-0410 | PEG8 | GAPDH | P | P3621 | UpCUpaCaUpgUUCCagUpaUpgaUpt | Propargyl |
| | M30 | | G | G3625 | UCaUaCUggaaCaUgUagaUpt | AlkDS-Ph |
| SB-0411 | PEG12 | GAPDH | P | P3621 | UpCUpaCaUpgUUCCagUpaUpgaUpt | Propargyl |
| | M30 | | G | G3625 | UCaUaCUggaaCaUgUagaUpt | AlkDS-Ph |
| SB-0412 | PEG24 | GAPDH | P | P3621 | UpCUpaCaUpgUUCCagUpaUpgaUpt | Propargyl |
| | M30 | | G | G3625 | UCaUaCUggaaCaUgUagaUpt | AlkDS-Ph |
| SB-0432 | Luc | | P | P3645 | gpCUaCaUUCUggagaCaUaUt | |
| | PEG24 | | G | G3639 | IR-UaUgUCUCCagaaUgUagCUt | AlkDS-Ph |
| SB-0433 | Luc | | P | P3571 | gpCUaCaUUCUggagaCaUaUt | |
| | | | G | G3573 | IR-UaUgUCUCCagaaUgUagCUt | |
| SB-0434 | Luc | | P | P3571 | gpCUaCaUUCUggagaCaUaUt | |
| | PEG24 | | G | G3639 | IR-UaUgUCUCCagaaUgUagCUt | AlkDS-Ph |
| SB-0435 | F6 | Luc | P | P3645 | gpCUaCaUUCUggagaCaUaUt | Propargyl |
| | PEG24 | | G | G3639 | IR-UaUgUCUCCagaaUgUagCUt | AlkDS-Ph |
| SB-0436 | F6 | Luc | P | P3571 | gpCUaCaUUCUggagaCaUaUt | Propargyl |
| | | | G | G3573 | IR-UaUgUCUCCagaaUgUagCUt | |
| SB-0437 | F6 | Luc | P | P3571 | gpCUaCaUUCUggagaCaUaUt | Propargyl |
| | PEG24 | | G | G3639 | IR-UaUgUCUCCagaaUgUagCUt | AlkDS-Ph |
| SB-0438 | NAG21 | Luc | P | P3571 | gpCUaCaUUCUggagaCaUaUt | Propargyl |
| | | | G | G3573 | IR-UaUgUCUCCagaaUgUagCUt | |
| SB-0439 | M6 | Luc | P | P3571 | gpCUaCaUUCUggagaCaUaUt | Propargyl |
| | | | G | G3573 | IR-UaUgUCUCCagaaUgUagCUt | |
| SB-0459 | NAG21 | AT3 | P | P3674 | DBCO-gpgUuAaCaCCAuUuAcUuCaa | DBCO |
| | | | G | G3508 | UUgAaGuAaAuggUgUuAaCcag | |
| SB-0464 | NAG21 | AT3 | P | P3507 | gpgUuAaCaCCAuUuAcUuCaa | |
| | | | G | G3646 | UUgAaGuAaAuggUgUuAaCcag | AlkDS-Ph |
| SB-0481 | NAG21 | AT3 | P | P3507 | gpgUuAaCaCCAuUuAcUuCaa | Propargyl |
| | | | G | G3681 | UUgAaGuAaAuggUgUuAaCcag | |
| SB-0482 | NAG21 | AT3 | P | P3507 | gpgUuAaCaCCAuUuAcUuCaa | |
| | | | G | G3682 | UUgAaGuAaAuggUgUuAaCcag | AlkDS-Ph |
| SB-0483 | NAG21 | AT3 | P | P3507 | gpgUuAaCaCCAuUuAcUuCaa | Propargyl |
| | | | G | G3686 | UUgAaGuAaAuggUgUuAaCcag | |
| SB-0484 | NAG21 | AT3 | P | P3507 | gpgUuAaCaCCAuUuAcUuCaa | Propargyl |
| | | | G | G3682 | UUpgAaGuAaAuggUgUuAaCcag | |
| SB-0506 | NAG21 | AT3 | P | P3507 | gpgUuAaCaCCAuUuAcUuCaa | Propargyl |
| | | | G | G3728 | UUgAaGuAaAurGgUgUuAaCcag | |
| SB-0507 | NAG21 | AT3 | P | P3507 | gpgUuAaCaCCAuUuAcUuCaa | Propargyl |
| | | | G | G3729 | UUgAaGuAaAugrGUgUuAaCcag | |
| SB-0508 | NAG21 | AT3 | P | P3507 | gpgUuAaCaCCAuUuAcUuCaa | Propargyl |
| | | | G | G3730 | UUgAaGuAaAuggrUgUuAaCcag | |
| SB-0509 | NAG21 | AT3 | P | P3507 | gpgUuAaCaCCAuUuAcUuCaa | Propargyl |
| | | | G | G3731 | UUgAaGuAaAuggUrGUuAaCcag | |
| SB-0510 | NAG21 | AT3 | P | P3507 | gpgUuAaCaCCAuUuAcUuCaa | Propargyl |
| | | | G | G3732 | UUgAaGuAaAuggUgrUuAaCcag | |
| SB-0511 | NAG21 | AT3 | P | P3507 | gpgUuAaCaCCAuUuAcUuCaa | Propargyl |
| | | | G | G3733 | UUgAaGuAaAuggUgUrUAaCcag | |
| SB-0512 | NAG21 | AT3 | P | P3507 | gpgUuAaCaCCAuUuAcUuCaa | Propargyl |
| | | | G | G3734 | UUgAaGuAaAuggUgUurAaCcag | |
| SB-0516 | NAG28 | AT3 | P | P3746 | gpgpUuAaCaCCAuUuAcUuCaa | Propargyl, |
| | | | G | G3508 | UUgAaGuAaAuggUgUuAaCcag | AlkDS-Ph |
| SB-0517 | NAG28 | AT3 | P | P3747 | gbgUuAaCaCCAuUUAcUuCaa | AlkDS-Ph |
| | | | G | G3508 | UUgAaGuAaAuggUgUuAaCcag | |
| SB-0518 | NAG28 | AT3 | P | P3748 | gbgUuAaCaCCAuUuACUUCaa | AlkDS-Ph |
| | | | G | G3508 | UUgAaGuAaAuggUgUuAaCcag | |
| SB-0519 | M30 | SIRPa | P | P3742 | apgGaGaAuAcGUUcCuCgAaU | Propargyl |
| | | | G | G3743 | aUuCgAgGaacGuAuUcUcCugbc | |
| SB-0520 | M30 | SIRPa | P | P3744 | gpaGaAuAcGuUcCuCgAaUUA | Propargyl |
| | | | G | G3745 | aUuCgAgGaAcGuAuUcUcCu | |
| SB-0522 | NAG21 | AT3 | P | P3749 | gpgsUuAaCaCCAuUuAcUuCaa | Propargyl |
| | | | G | G3508 | UUgAaGuAaAuggUgUuAaCcag | |
| SB-0523 | NAG21 | AT3 | P | P3750 | gpgUsuAaCaCCAuUuAcUuCaa | Propargyl |
| | | | G | G3508 | UUgAaGuAaAuggUgUuAaCcag | |
| SB-0524 | NAG21 | AT3 | P | P3751 | gpgUuAsaCaCCAuUuAcUuCaa | Propargyl |
| | | | G | G3508 | UUgAaGuAaAuggUgUuAaCcag | |
| SB-0525 | NAG21 | AT3 | P | P3752 | gpgUuAaCaCCAuUuAscUuCaa | Propargyl |
| | | | G | G3508 | UUgAaGuAaAuggUgUuAaCcag | |
| SB-0526 | NAG21 | AT3 | P | P3753 | gpgUuAaCaCCAuUuAcUsuCaa | Propargyl |
| | | | G | G3508 | UUgAaGuAaAuggUgUuAaCcag | |
| SB-0527 | NAG21 | AT3 | P | P3754 | gpgUsuAsaCaCCAuUuAscUsuCaa | Propargyl |
| | | | G | G3508 | UUgAaGuAaAuggUgUuAaCcag | |
| SB-0528 | NAG21 | AT3 | P | P3507 | gpgUuAaCaCCAuUuAcUuCaa | Propargyl |
| | | | G | G3755 | UsUsgAaGuAaAuggUgUuAaCcag | |
| SB-0529 | NAG21 | AT3 | P | P3507 | gpgUuAaCaCCAuUuAcUuCaa | Propargyl |
| | | | G | G3756 | UUsgAaGuAaAuggUgUuAaCcag | |
| SB-0530 | NAG21 | AT3 | P | P3507 | gpgUuAaCaCCAuUuAcUuCaa | Propargyl |
| | | | G | G3757 | UUgAasGuAaAuggUgUuAaCcag | |
| SB-0531 | NAG21 | AT3 | P | P3507 | gpgUuAaCaCCAuUuAcUuCaa | Propargyl |
| | | | G | G3758 | UUgAaGsuAaAuggUgUuAaCcag | |
| SB-0532 | NAG21 | AT3 | P | P3507 | gpgUuAaCaCCAuUuAcUuCaa | Propargyl |
| | | | G | G3759 | UUgAaGuAaAuggUgUusAaCcag | |
| SB-0533 | NAG21 | AT3 | P | P3507 | gpgUuAaCaCCAuUuAcUuCaa | Propargyl |
| | | | G | G3760 | UUsgAasGuAaAuggUgUusAaCcag | |
| SB-0535 | NAG21 | AT3 | P | P3862 | gpgUuAaCaCCAuUuAcUuCaa-PEG6- | Propargyl |
| | | | | | gbgUuAaCaCCAuUuAcUuCaa | |
| | | | G | G3508 | (UUgAaGuAaAuggUgUuAaCcag)2 | |
| SB-0538 | NAG21 | AT3 | P | P3777 | DBCO-gmgUuAaCaCCAuUuAcUuCama | DBCO |
| | | | G | G3778 | UUgAaGuAaAuggUgUuAaCcamg | |
| SB-0539 | NAG21 | AT3 | P | P3777 | DBCO-gmgUuAaCaCCAuUuAcUuCama | DBCO |
| | | | G | G3779 | UmUgAaGuAaAuggUgUuAaCcamg | |
| SB-0560 | NAG21 | AT3 | P | P3780 | gpsgUuAaCaCCAuUuAcUuCasa | Propargyl |
| | | | G | G3781 | UsUgAaGuAaAuggUgUuAaCcasg | |
| SB-0561 | NAG21 | AT3 | P | P3782 | gpsgsUuAaCaCCAuUuAcUuCsasa | Propargyl |
| | | | G | G3783 | UsUsgAaGuAaAuggUgUuAaCcsasg | |
| SB-0588 | NAG21 | AT3 | P | P3784 | DBCO-gsgsUuAaCaCCAuUuAcUuCsasa | DBCO |
| | | | G | G3785 | UsUsgAaGuAaAuggUgUuAaCcsasg | |
| SB-0589 | NAG21 | AT3 | P | P3786 | DBCO-gsgUuAaCaCCAuUuAcUuCasa | DBCO |
| | | | G | G3787 | UsUgAaGuAaAuggUgUuAaCcasg | |
| SB-0590 | NAG28 | AT3 | P | P3794 | gbgUuAaCaCCAuUuAcUUpCpapa | Propargyl |
| | | | G | G3508 | UUgAaGuAaAuggUgUuAaCcag | |
| SB-0591 | NAG28 | AT3 | P | P3795 | gbgUuAaCaCCAuUuACpUUpCapa | Propargyl |
| | | | G | G3508 | UUgAaGuAaAuggUgUuAaCcag | |
| SB-0592 | NAG28 | AT3 | P | P3796 | gbgUuAaCaCCAuUuAcUuCpapa | Propargyl |
| | NAG28 | | G | G3646 | UUgAaGuAaAuggUgUuAaCcag | AlkDS-Ph |
| SB-0593 | NAG28 | AT3 | P | P3797 | gbgUuAaCaCCAuUuAcUUpCapa | Propargyl |
| | NAG28 | | G | G3646 | UUgAaGuAaAuggUgUuAaCcag | |
| SB-0594 | NAG21 | AT3 | P | P3507 | gpgUuAaCaCCAuUuAcUuCaa | Propargyl |
| | | | G | G3799 | UUgAaGuAaAuggUgUuAaCCag | |
| SB-0595 | NAG28 | AT3 | P | P3798 | gbgUuAaCaCCAuUuAcUuCaa | |
| | | | G | G3799 | UUgAaGuAaAuggUgUuAaCCag | AlkDS-Ph |
| SB-0596 | NAG21 | AT3 | P | P3507 | gpgUuAaCaCCAuUuAcUuCaa | Propargyl |
| | | | G | G3800 | UUgAaGuAaAuggUgUuaaCcag | |
| SB-0597 | NAG28 | AT3 | P | P3798 | gbgUuAaCaCCAuUuAcUuCaa | |
| | | | G | G3800 | UUgAaGuAaAuggUgUuaaCcag | AlkDS-Ph |
| SB-0598 | NAG28 | AT3 | P | P3507 | gpgUuAaCaCCAuUuAcUuCaa | Propargyl |
| | NAG28 | | G | G3801 | UUgAaGuAaAuggUgUuAaCCag | AlkDS-Ph |
| SB-0599 | NAG28 | AT3 | P | P3507 | gpgUuAaCaCCAuUuAcUuCaa | Propargyl |
| | NAG28 | | G | G3802 | UUgAaGuAaAuggUgUuAaCcag | AlkDS-Ph |
| SB-0599 | NAG28 | AT3 | P | P3507 | gpgUuAaCaCCAuUuAcUuCaa | Propargyl |
| | NAG28 | | G | G3802 | UUgAaGuAaAuggUgUuAaCcag | AlkDS-Ph |
| SB-0600 | NAG21-BIL5 | AT3 | P | P3507 | (gpgUuAaCaCCAuUuAcUuCaa)2 | |
| | (Split Linker) | | G | G3671 | (UDUgAaGuAaAuggUgUuAaCcag)2 | AldDS-Ph |
| SB-0601 | NAG21 | AT3 | P | P3805 | gsgsUuAaCaCCAuUuAcUuCasas-Alk | Alk |
| | | | G | G3504 | usUsgAaGuAaAuggUgUuAaCcsasg | |
| SB-0602 | NAG21 | AT3 | P | P3806 | gbgUuAaCaCCAuUuAcUuCaA-Alk | Alk |
| | | | G | G3508 | UUgAaGuAaAuggUgUuAaCcag | |
| SB-0603 | NAG21 | AT3 | P | P3807 | gbgUuAaCaCCAuUuAcUuCaA-Alk | Alk |
| | | | G | G3508 | UUgAaGuAaAuggUgUuAaCcag | |
| SB-0604 | NAG21 | AT3 | P | P3808 | gbgUuAaCaCCAuUuAcUuCaa-Alk | Alk |
| | | | G | G3508 | UUgAaGuAaAuggUgUuAaCcag | |
| SB-0608 | NAG21 | AT3 | P | P3507 | gpgUuAaCaCCAuUuAcUuCaa | Propargyl |
| | | | G | G3809 | UUrGrAaGuAaAuggUgUuArArCcag | |
| SB-0609 | NAG21 | AT3 | P | P3507 | gpgUuAaCaCCAuUuAcUuCaa | Propargyl |
| | | | G | G3810 | UUrGrArAGuAaAuggUgUurArArCcag | |
| SB-0610 | NAG21 | AT3 | P | P3507 | gpgUuAaCaCCAuUuAcUuCaa | Propargyl |
| | | | G | G3811 | UurGArAGrUArAArUgrGUrGUrUArACcag | |
| SB-0611 | NAG21 | AT3 | P | P3789 | DBCO-gpgUuAaCaCCAuUuAcUuCapa | DBCO |
| | | | G | G3790 | UpUgAaGuAaAuggUgUuAaCcapg | |
| SB-0612 | NAG21, EtOH | AT3 | P | P3789 | DBCO-gpUuAaCaCCAuUuAcUuCapa | DBCO, Propargyl |
| | EtOH | | G | G3790 | UpUgAaGuAaAuggUgUuAaCcapg | Propargyl |
| SB-0613 | NAG21, HOAc | AT3 | P | P3789 | DBCO-gpUuAaCaCCAuUuAcUuCapa | DBCO, Propargyl |
| | HOAc | | G | G3790 | UpUgAaGuAaAuggUgUuAaCcapg | Propargyl |
| SB-0614 | NAG21, dGlucose | AT3 | P | P3789 | DBCO-gpgUuAaCaCCAuUuAcUuCapa | DBCO, Propargyl |
| | dGlucose | | G | G3790 | UpUgAaGuAaAuggUgUuAaCcapg | Propargyl |
| SB-0615 | NAG21, PEG3-Acid | AT3 | P | P3789 | DBCO-gpgUuAaCaCCAuUuAcUuCapa | DBCO, Propargyl |
| | PEG3-Acid | | G | G3790 | UpUgAaGuAaAuggUgUuAaCcapg | Propargyl |
| SB-0616 | NAG21, mGlucose | AT3 | P | P3789 | DBCO-gpgUuAaCaCCAuUuAcUuCapa | DBCO, Propargyl |
| | mGlucose | | G | G3790 | UpUgAaGuAaAuggUgUuAaCcapg | Propargyl |
| SB-0617 | NAG21, bGlucose | AT3 | P | P3789 | DBCO-gpUuAaCaCCAuUuAcUuCapa | DBCO, Propargyl |
| | bGlucose | | G | G3790 | UpUgAaGuAaAuggUgUuAaCcapg | Propargyl |
| SB-0618 | NAG21, tGlucose | AT3 | P | P3789 | DBCO-gpUuAaCaCCAuUuAcUuCapa | DBCO, Propargyl |
| | tGlucose | | G | G3790 | UpUgAaGuAaAuggUgUuAaCcapg | Propargyl |
| SB-0619 | NAG26 | AT3 | P | P3795 | gbgUuAaCaCCAuUuACpUUpCapa | Propargyl |
| | | | G | G3508 | UUgAaGuAaAuggUgUuAaCcag | |
| SB-0620 | NAG26 | AT3 | P | P3796 | gbgUuAaCaCCAuUuAcUuCpapa | Propargyl |
| | NAG26 | | G | G3646 | UUgAaGuAaAuggUgUuAaCcag | AlkDS-Ph |
| SB-0621 | NAG26 | AT3 | P | P3798 | gbgUuAaCaCCAuUuAcUuCaa | |
| | | | G | G3800 | UUgAaGuAaAuggUgUuaaCcag | AlkDS-Ph |
| SB-0622 | NAG26 | AT3 | P | P3507 | gpgUuAaCaCCAuUuAcUuCaa | Propargyl |
| | NAG26 | | G | G3802 | UUgAaGuAaAuggUgUuAaCcag | AlkDS-Ph |
| SB-0623 | Folate | GAPDH | P | P3635 | NH2-UpCUaCaUgUUCCagUaUgaUpt | NH2 |
| | bGlucose | | G | G3812 | UCaUaCUggaaCaUgUagaUt | AlkDS-Ph |
| SB-0624 | Folate | GAPDH | P | P3635 | NH2-UpCUaCaUgUUCCagUaUgaUpt | NH2 |
| | tGlucose | | G | G3812 | UCaUaCUggaaCaUgUagaUt | AlkDS-Ph |
| SB-0625 | Folate | Luc | P | P3632 | NH2-gbCUaCaUUCUggagaCaUaUpt | NH2 |
| | bGlucose | | G | G3667 | UaUgUCUCCagaaUgUagCUt | AlkDS-Ph |
| SB-0626 | Folate | Luc | P | P3632 | NH2-gbCUaCaUUCUggagaCaUaUpt | NH2 |
| | tGlucose | | G | G3667 | UaUgUCUCCagaaUgUagCUt | AlkDS-Ph |
| SB-0627 | NAG21 | AT3 | P | P3817 | gpgUuAaCaCCAuUuAcUuCaa | Propargyl |
| | | | G | G3818 | UUgAaGuAaAuggUgUuAaCcagb | |
| SB-0639 | NAG21 | AT3 | P | P3833 | gpgUuAaCaCCAuUuAcUuCaa-S-S- | Propargyl |
| | | | | | gbgUuAaCaCCAuUuAcUuCaa | |
| | | | G | G3508 | (UUgAaGuAaAuggUgUuAaCcag)2 | |
| SB-0640 | NAG21 | AT3 | P | P3834 | gpgUuAaCaCCAuUuAcUuCaa-S-S- | Propargyl |
| | | | | | ggUuAaCaCCAuUuAcUuCaa | |
| | | | G | G3508 | (UUgAaGuAaAuggUgUuAaCcag)2 | |
| SB-0641 | NAG28 | AT3 | P | P3814 | qpqpUpuAaCaCCAuUuAcUuCaa | Propargyl |
| | | | G | G3508 | UUgAaGuAaAuggUgUuAaCcag | |
| SB-0642 | NAG28 | AT3 | P | P3815 | gbgUuAaCaCCAuUpUpapcUuCaa | Propargyl |
| | | | G | G3508 | UUgAaGuAaAuggUgUuAaCcag | |
| SB-0643 | NAG28 | AT3 | P | P3816 | gbgUuAaCaCCAuUuACpUpUpCaa | Propargyl |
| | | | G | G3508 | UUgAaGuAaAuggUgUuAaCcag | |
| SB-0665 | NAG21 | AT3 | P | P3840 | gpguuaacaCcAuuuacuucaa | Propargyl |
| | | | G | G3842 | UUGaAgUAaAuggUgUuAaccag | |
| SB-0666 | NAG21 | AT3 | P | P3841 | gpgUuAaCaCCAuUuacuucaa | Propargyl |
| | | | G | G3843 | UUgAaguAaAuggUgUuaaCcag | |
| SB-0672 | NAG21 | AT3 | P | P3836 | gprGrUrUrArArCrArCrCrArUrUrUrArCrUrUrCarA | Propargyl |
| | | | G | G3838 | UrUrGrArArGrUrArArArUrGrGrUrGrUrUrArArCrCarG | |
| SB-0673 | NAG21 | AT3 | P | P3507 | gpgUuAaCaCCAuUuAcUuCaa | Propargyl |
| | | | G | G3838 | UrUrGrArArGrUrArArArUrGrGrUrGrUrUrArArCrCarG | |
| SB-0674 | NAG21 | AT3 | P | P3836 | gprGrUrUrArArCrArCrCrArUrUrUrArCrUrUrCarA | Propargyl |
| | | | G | G3508 | UUgAaGuAaAuggUgUuAaCcag | |
| SB-0675 | NAG21 | AT3 | P | P3837 | gpgrUurAarCarCrCrAurUurAcrUurCarA | Propargyl |
| | | | G | G3839 | UrUgrAarGurAarAuggrUgrUurAarCcag | |
| SB-0676 | NAG21 | AT3 | P | P3507 | gpgUuAaCaCCAuUuAcUuCaa | Propargyl |
| | | | G | G3839 | UrUgrAarGurAarAuggrUgrUurAarCcag | |
| SB-0677 | NAG21 | AT3 | P | P3837 | gprUurAarCarCrCrAurUurAcrUurCarA | Propargyl |
| | | | G | G3508 | UUgAaGuAaAuggUgUuAaCcag | |
| SB-0678 | NAG21 | AT3 | P | P3507 | gpgUuAaCaCCAuUuAcUuCaa | Propargyl |
| | | | G | G3846 | UIUgAaGuAaAuggUgUuAaCcag | |
| SB-0706 | NAG21 | AT3 | P | P3872 | DBCO-gBgUuAaCaCCAuUuAcUuCaBa | DBCO |
| | | | G | G3873 | UBUgAaGuAaAuggUgUuAaCcaBg | |
| SB-0707 | NAG21 | AT3 | P | P3872 | DBCO-gBgUuAaCaCCAuUuAcUuCaBa | DBCO |
| | | | G | G3874 | UUgAaGuAaAuggUgUuAaCcaBg | |
| SB-0720 | NAG21 | AT3 | P | P3507 | gpgUuAaCaCCAuUuAcUuCaa | Propargyl |
| | P42 | | G | G3646 | UUgAaGuAaAuggUgUuAaCcag | Alkyne Disulfide |
| SB-0721 | NAG21 | AT3 | P | P3507 | gpgUuAaCaCCAuUuAcUuCaa | Propargyl |
| | P42 | | G | G3801 | UUgAaGuAaAuggUgUuAaCCag | Alkyne Disulfide |
| SB-0722 | NAG21 | AT3 | P | P3507 | gpgUuAaCaCCAuUuAcUuCaa | Propargyl |
| | P42 | | G | G3802 | UUgAaGuAaAuggUgUuAaCcag | Alkyne Disulfide |
| SB-0723 | NAG21 | AT3 | P | P3507 | gpgUuAaCaCCAuUuAcUuCaa | Propargyl |
| | P42 | | G | G3799 | UUgAaGuAaAuggUgUuAaCCag | Alkyne Disulfide |
| SB-0724 | NAG21 | AT3 | P | P3507 | gpgUuAaCaCCAuUuAcUuCaa | Propargyl |
| | P42 | | G | G3800 | UUgAaGuAaAuggUgUuaaCCag | Alkyne Disulfide |
| SB-0730 | NAG21 | AT3 | P | P3890 | gpguuaacarCcrAuuuacuucaa | Propargyl |
| | | | G | G3891 | UrUrGarAgrUrAarAuggrUgrUurAaccag | |
| SB-0731 | NAG21 | AT3 | P | P3892 | gpgrUurAarCarCrCrAurUuacuucaa | Propargyl |
| | | | G | G3893 | UrUgrAagurAarAuggrUgrUuaarCcag | |
| SB-0732 | NAG21 | AT3 | P | P3894 | gpgrUurAarCarCrCrAurUurAcrUurCarA | Propargyl |
| | | | G | G3897 | UrUgrAarGurAarAuggrUgrUurAarCcag | |
| SB-0733 | NAG21 | AT3 | P | P3895 | gpgrUurAarCarCrCrAurUurAcUurCarA | Propargyl |
| | | | G | G3897 | UrUgrAarGurAarAuggrUgrUurAarCcag | |
| SB-0734 | NAG21 | AT3 | P | P3896 | gpgrUurAarCarCrCrAurUurAcUurCarA | Propargyl |
| | | | G | G3897 | UrUgrAarGurAarAuggrUgrUurAarCcag | |
| SB-0750 | NAG21 | AT3 | P | P3921 | gpgUuAaCaCCAuUuAcUuCMaa | Propargyl |
| | | | G | G3922 | MUUgAaGuAaAuggUgUuAaCcMag | |
| SB-0751 | NAG21 | AT3 | P | P3921 | gpgUuAaCaCCAuUuAcUuCMaa | Propargyl |
| | | | G | G3923 | UUgAaGuAaAuggUgUuAaCcMag | |

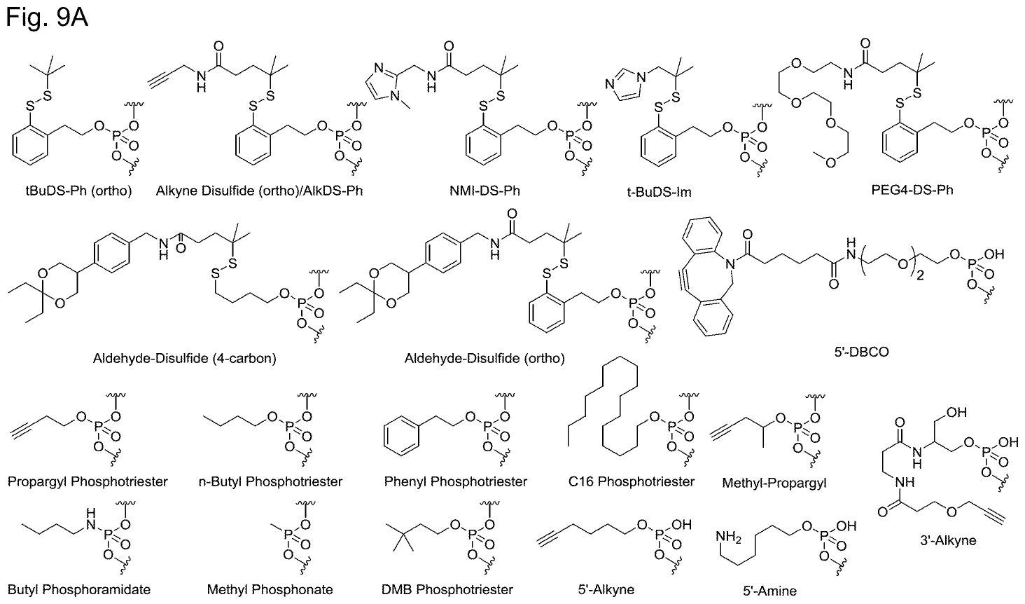

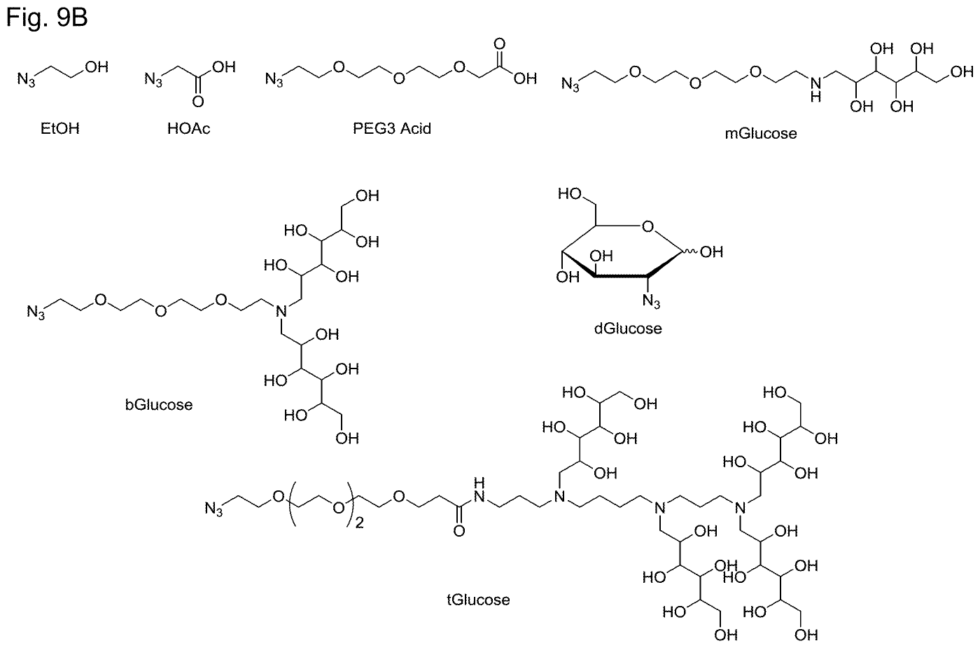

In Table 7: UPPER CASE = 2′F; lower case = 2′OMe; M = 2′methoxyethyl; italics = tBuDS-Ph(ortho); UNDERLINE = Alkyne Disulfide (ortho)/AlkDS-Ph; UNDERLINE = conjugated location; Alk = 3′ alkyne; Hex = 5′ Hexynyl; NH2 = 5′ amine; s = phosphorothioate; p = homopropargyl phosphotriester; d = DMB phosphotriester; b = n-butyl phosphotriester; P = phenyl phosphotriester; I = NMI-DS-Ph; p = PEG4-DS-Ph; mp = methyl-homopropargyl; B = butyl phosphoramidate; Ph = 5′ phosphate; h = C16 phosphotriester; DBCO = 5′DBCO Copper-free Conjugation; IR = infrared imaging dye; r = 2′ OH; m = methylphosphonate; I = tBuDS-Im; S-S = C6 disulfide spacer; EtOH = ethanol-azide; HOAc = acetic acid-azide; PEG3-Acid = Acid-PEG3-Azide; dGlucose = deoxy-Glucose-Azide; bGlucose = bis-Glucose-Azide; tGlucose = tetra-Glucose-Azide; BIL5 = split linker formed using BIL5; SP1L = spermine-azide; DS means disulfide; For the purpose of this table, Ph means phenethyl, Propargyl means homopropargyl, Methyl-Propargyl means pent-5-yn-2-yl. The structures of these groups are as described above and provided in FIGS. 9Aand 9B.

For Table 7, SB-0535 includes PEG6 spacer connecting 3′ end of the first passenger strand to 5′ end of the second passenger strand. The first passenger strand is hybridized to the first guide strand, and the second passenger strand is hybridized to the second guide strand. The two guide strands are not directly covalently bonded to each other. The PEG6 spacer was formed from the following phosphoramidite:

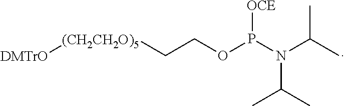

SB-0600 includes NAG21-BIL5 linker conjugated to two guide strands as shown in the table above. The first guide strand is hybridized to the first passenger strand, and the second guide strand is hybridized to the second passenger strand. The two passenger strands are not directly covalently bonded to each other. SB-0639 and SB-0640 include C6 Disulfide spacer connecting 3′ end of the first passenger strand to 5′ end of the second passenger strand. The first passenger is hybridized to the first guide strand, and the second passenger strand is hybridized to the second guide strand. The two guide strands are not directly covalently bonded to each other. The C6 Disulfide spacer was formed from the following phosphoramidite:

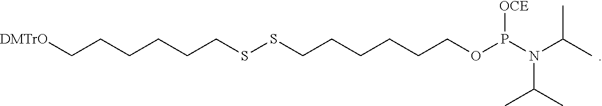

TABLE 8

| | | | |
|---|---|---|---|
| | | | yield (%) |
| | Duplex | ss | w/>90-95% |
| ApoB ss sequences | SB # | SB # | purity |

Passenger Strand (5′-3′)

| | | | |
|---|---|---|---|
| UCAUCACACUGAAUACCAAUT | SB-0165 | P3373 | Control |
| UCAUCACACUGAAUACCAAUT | SB-0166 | P3371 | 44.7 |
| UCAUCACACUGAAUACCAAUT | SB-0167 | P3464 | 3.2 |
| UCAUCACACUGAAUACCAAUT | SB-0168 | P3465 | 68.6 |
| UCAUCACACUGAAUACCAAUT | SB-0169 | P3466 | 52 |
| UCAUCACACUGAAUACCAAUT | SB-0170 | P3467 | 27.8 |
| UCAUCACACUGAAUACCAAUT | SB-0171 | P3468 | 56.2 |
| UCAUCACACUGAAUACCAAUT | SB-0172 | P3469 | 13.3 |
| UCAUCACACUGAAUACCAAUT | SB-0173 | P3470 | 55.1 |
| UCAUCACACUGAAUACCAAUT | SB-0174 | P3471 | 30.6 |
| UCAUCACACUGAAUACCAAUT | SB-0175 | P3472 | 56.0 |
| UCAUCACACUGAAUACCAAUT | SB-0176 | P3473 | 24.0 |
| UCAUCACACUGAAUACCAAUT | SB-0177 | P3474 | 52.7 |
| UCAUCACACUGAAUACCAAUT | SB-0178 | P3475 | 44.6 |
| UCAUCACACUGAAUACCAAUT | SB-0179 | P3476 | 51.4 |
| UCAUCACACUGAAUACCAAUT | SB-0180 | P3477 | 45.6 |
| UCAUCACACUGAAUACCAAUT | SB-0181 | P3478 | 44.4 |
| UCAUCACACUGAAUACCAAUT | SB-0182 | P3479 | 45.6 |
| UCAUCACACUGAAUACCAAUT | SB-0183 | P3480 | 35.2 |
| UCAUCACACUGAAUACCAAUT | SB-0184 | P3481 | 41.4 |
| UCAUCACACUGAAUACCAAUT | SB-0185 | P3482 | 51.5 |

Guide Strand (3′- 5′)

| | | | |
|---|---|---|---|
| TUAGUAGUGUGACUUAUGGUU | SB-0165 | G3292 | Control |
| TUAGUAGUGUGACUUAUGGUU | SB-0186 | G3483 | 61.0 |
| TUAGUAGUGUGACUUAUGGUU | SB-0187 | G3484 | 56.7 |
| TUAGUAGUGUGACUUAUGGUU | SB-0188 | G3485 | 24.0 |
| TUAGUAGUGUGACUUAUGGUU | SB-0189 | G3486 | 54.1 |
| TUAGUAGUGUGACUUAUGGUU | SB-0190 | G3487 | 35.5 |
| TUAGUAGUGUGACUUAUGGUU | SB-0191 | G3488 | 49.6 |
| TUAGUAGUGUGACUUAUGGUU | SB-0192 | G3489 | 44.6 |
| TUAGUAGUGUGACUUAUGGUU | SB-0193 | G3490 | 46.7 |
| TUAGUAGUGUGACUUAUGGUU | SB-0194 | G3491 | 56.4 |
| TUAGUAGUGUGACUUAUGGUU | SB-0195 | G3492 | 52.0 |
| TUAGUAGUGUGACUUAUGGUU | SB-0196 | G3493 | 49.1 |
| TUAGUAGUGUGACUUAUGGUU | SB-0197 | G3494 | 45.5 |
| TUAGUAGUGUGACUUAUGGUU | SB-0198 | G3495 | 54.3 |
| TUAGUAGUGUGACUUAUGGUU | SB-0199 | G3496 | 49.0 |
| TUAGUAGUGUGACUUAUGGUU | SB-0200 | G3497 | 55.5 |
| TUAGUAGUGUGACUUAUGGUU | SB-0201 | G3498 | 47.1 |
| TUAGUAGUGUGACUUAUGGUU | SB-0202 | G3499 | 52.8 |
| TUAGUAGUGUGACUUAUGGUU | SB-0203 | G3500 | 42.3 |
| TUAGUAGUGUGACUUAUGGUU | SB-0204 | G3501 | 60.0 |
| TUAGUAGUGUGACUUAUGGUU | SB-0205 | G3502 | 53.0 |

In table 8, BOLD indicates a nucleotide having o-(t-butyldithio)phenethyl group bonded to 3′-phosphate; UNDERLINED indicates a nucleotide having propargyl group bonded to 3′-phosphate. The duplexes with Watson-Crick alignment of passenger and guide strands were prepared by annealing the strands under standard conditions as described herein.

The duplexes produced from strands shown in Table 8 are listed in Table 9.

TABLE 9

| Compound # | Structure |
|---|---|
| SB-0165 | UCAUCACACUGAAUACCAAUt |
| | tUAGUAGUGUGACUUAUGGUU |
| SB-0166 | UCAUCACACUGAAUACCAAUt |
| | tUAGUAGUGUGACUUAUGGUU |
| SB-0167 | UCAUCACACUGAAUACCAAUt |
| | tUAGUAGUGUGACUUAUGGUU |
| SB-0168 | UCAUCACACUGAAUACCAAUt |
| | tUAGUAGUGUGACUUAUGGUU |
| SB-0169 | UCAUCACACUGAAUACCAAUt |
| | tUAGUAGUGUGACUUAUGGUU |
| SB-0170 | UCAUCACACUGAAUACCAAUt |
| | tUAGUAGUGUGACUUAUGGUU |
| SB-0171 | UCAUCACACUGAAUACCAAUt |
| | tUAGUAGUGUGACUUAUGGUU |
| SB-0172 | UCAUCACACUGAAUACCAAUt |
| | tUAGUAGUGUGACUUAUGGUU |
| SB-0173 | UCAUCACACUGAAUACCAAUt |
| | tUAGUAGUGUGACUUAUGGUU |
| SB-0174 | UCAUCACACUGAAUACCAAUt |
| | tUAGUAGUGUGACUUAUGGUU |
| SB-0175 | UCAUCACACUGAAUACCAAUt |
| | tUAGUAGUGUGACUUAUGGUU |
| SB-0176 | UCAUCACACUGAAUACCAAUt |
| | tUAGUAGUGUGACUUAUGGUU |
| SB-0177 | UCAUCACACUGAAUACCAAUt |
| | tUAGUAGUGUGACUUAUGGUU |
| SB-0178 | UCAUCACACUGAAUACCAAUt |
| | tUAGUAGUGUGACUUAUGGUU |
| SB-0179 | UCAUCACACUGAAUACCAAUt |
| | tUAGUAGUGUGACUUAUGGUU |
| SB-0180 | UCAUCACACUGAAUACCAAUt |
| | tUAGUAGUGUGACUUAUGGUU |
| SB-0181 | UCAUCACACUGAAUACCAAUt |
| | tUAGUAGUGUGACUUAUGGUU |
| SB-0182 | UCAUCACACUGAAUACCAAUt |
| | tUAGUAGUGUGACUUAUGGUU |
| SB-0183 | UCAUCACACUGAAUACCAAUt |
| | tUAGUAGUGUGACUUAUGGUU |
| SB-0184 | UCAUCACACUGAAUACCAAUt |
| | tUAGUAGUGUGACUUAUGGUU |
| SB-0185 | UCAUCACACUGAAUACCAAUt |
| | tUAGUAGUGUGACUUAUGGUU |
| SB-0186 | UCAUCACACUGAAUACCAAUt |
| | tUAGUAGUGUGACUUAUGGUU |
| SB-0187 | UCAUCACACUGAAUACCAAUt |
| | tUAGUAGUGUGACUUAUGGUU |
| SB-0188 | UCAUCACACUGAAUACCAAUt |
| | tUAGUAGUGUGACUUAUGGUU |
| SB-0189 | UCAUCACACUGAAUACCAAUt |
| | tUAGUAGUGUGACUUAUGGUU |
| SB-0190 | UCAUCACACUGAAUACCAAUt |
| | tUAGUAGUGUGACUUAUGGUU |
| SB-191 | UCAUCACACUGAAUACCAAUt |
| | tUAGUAGUGUGACUUAUGGUU |
| SB-0192 | UCAUCACACUGAAUACCAAUt |
| | tUAGUAGUGUGACUUAUGGUU |
| SB-0193 | UCAUCACACUGAAUACCAAUt |
| | tUAGUAGUGUGACUUAUGGUU |
| SB-0194 | UCAUCACACUGAAUACCAAUt |
| | tUAGUAGUGUGACUUAUGGUU |
| SB-0195 | UCAUCACACUGAAUACCAAUt |
| | tUAGUAGUGUGACUUAUGGUU |
| SB-0196 | UCAUCACACUGAAUACCAAUt |
| | tUAGUAGUGUGACUUAUGGUU |
| SB-0197 | UCAUCACACUGAAUACCAAUt |
| | tUAGUAGUGUGACUUAUGGUU |
| SB-0198 | UCAUCACACUGAAUACCAAUt |
| | tUAGUAGUGUGACUUAUGGUU |
| SB-0199 | UCAUCACACUGAAUACCAAUt |
| | tUAGUAGUGUGACUUAUGGUU |
| SB-0200 | UCAUCACACUGAAUACCAAUt |
| | tUAGUAGUGUGACUUAUGGUU |
| SB-0201 | UCAUCACACUGAAUACCAAUt |
| | tUAGUAGUGUGACUUAUGGUU |
| SB-0202 | UCAUCACACUGAAUACCAAUt |
| | tUAGUAGUGUGACUUAUGGUU |
| SB-0203 | UCAUCACACUGAAUACCAAUt |
| | tUAGUAGUGUGACUUAUGGUU |
| SB-0204 | UCAUCACACUGAAUACCAAUt |
| | tUAGUAGUGUGACUUAUGGUU |
| SB-0205 | UCAUCACACUGAAUACCAAUt |
| | tUAGUAGUGUGACUUAUGGUU |

Any of the groups disclosed herein may be linked to an internucleotide bridging phosphate or a terminal phosphate through one of the following non-limiting exemplary groups:

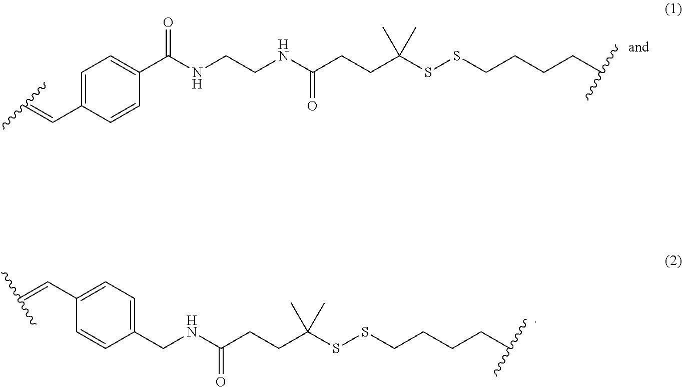 [SMILES: *CCCCSSC(C)(C)CCC(=O)NCCNC(=O)c1ccc(C=*)cc1 ; *CCCCSSC(C)(C)CCC(=O)NCc1ccc(C=*)cc1]

Other polynucleotides of the invention may be prepared according to the methods described herein. Such polynucleotides may be as follows:

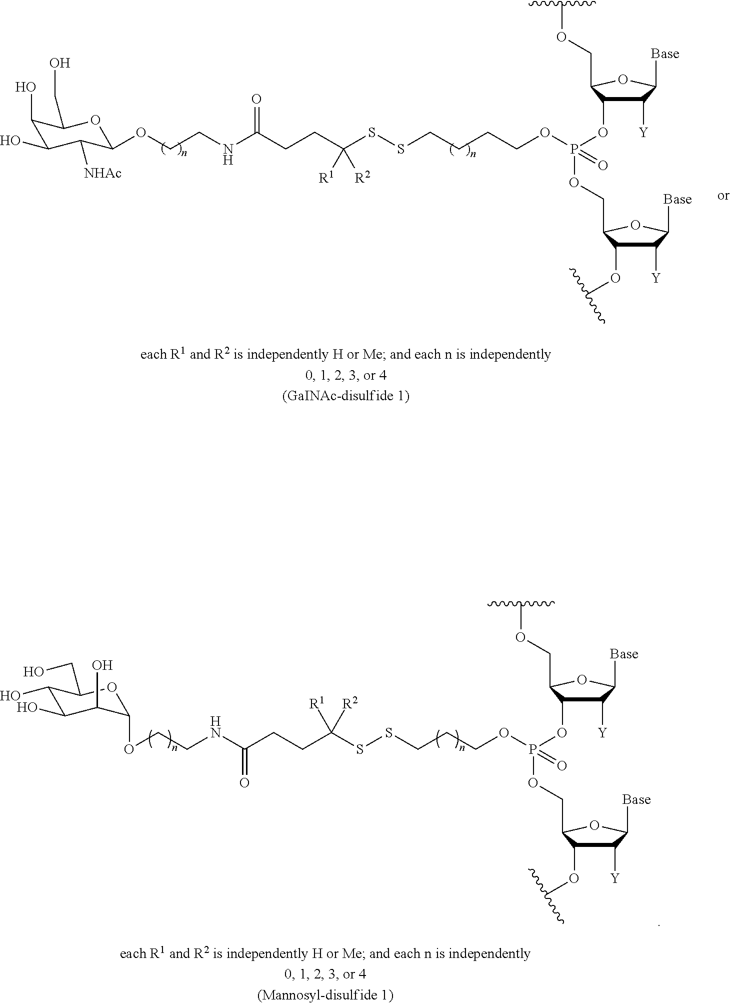

Polynucleotides containing auxiliary moieties directly bound to the disulfide linkage may also be prepared; exemplary polynucleotides are shown below:

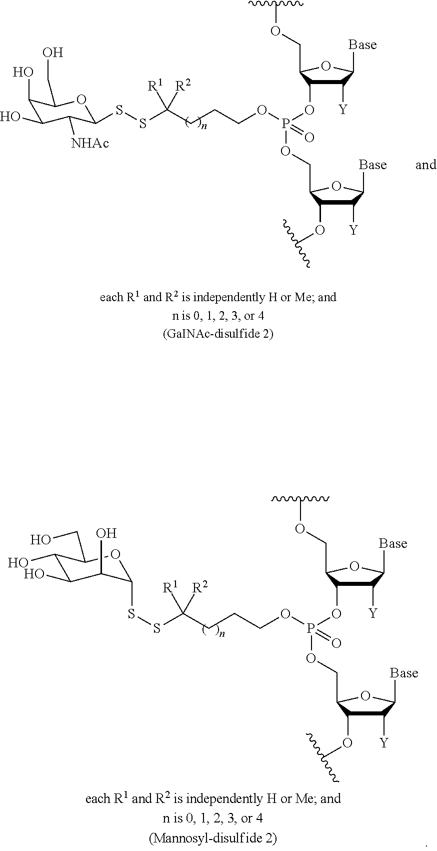

### Example 2. In Vitro Activity Assays

### Suppression of Luciferase Expression

Polynucleotides targeting the luciferase gene (GL3) were synthesized and were used to generate the polynucleotide constructs having bioreversible groups (disulfide phosphodiester or disulfide phosphotriester).

To assess the in vitro activity of these disulfide phosphotriesters, human ovarian SKOV-3 cells, stably expressing luciferase (GL3) were utilized. Cells were grown in McCoy's 5A culture medium (life technologies) supplemented with 10% fetal bovine serum (FBS), 100 μg/ml of streptomycin, and 100 U/ml of penicillin. Cells (1×10⁴/well) were plated in 96-well microtiter plates and incubated overnight at 37° C. under 5% CO₂.

Control: The control siRNAs targeting the luciferase gene or a non-targeting control gene were transfected into cells at the indicated concentrations (typically 0.01-30 nM) using lipofectamine RNAiMax (Life Technologies) according to the manufacturer's recommendations.

Polynucleotide Constructs of the Invention: The polynucleotide constructs were added to cells and incubated for two hours, after which an equal volume of OptiMEM (life technologies) containing 4% FBS was added and the cells were incubated for 24-48 hours. The cells were then lysed and the intracellular luciferase activity was measured after the addition of luciferin (Britelite™, Perkin Elmer) and the luminescence signal was captured using Victor2™ luminometer (Perkin Elmer). Cellular toxicity was assessed using the CellTiterFluor™ assay kit (Promega) and the knockdown f the luciferase gene was corrected for cellular toxicity and was expressed as percent of vehicle control treated wells. Luciferase knock-down EC₅₀ values were generated using GraphPad Prism Software.

Results of this assay for the hybridized polynucleotide of the invention (SEQ ID NOs: 112 and 113) are shown in Table 10 (for the structures see FIG. 1A). In Table 10, R⁴ is 2-(benzylaminocarbonyl)ethyl.

TABLE 10

| | | | | | | | EC50^{(a)} | (nM) | EC50^{(b)} | (nM) |
|---|---|---|---|---|---|---|---|---|---|---|
| Entry | R | R¹ | R² | R³ | R⁴ | n | 24 h | 48 h | 24 h | 48 h |
| 1 | Me | H | Me | H | H | 1 | 0.76 | 0.33 | 0.42 | 0.18 |
| 2 | Me | H | CH₂OH | H | H | 1 | ND | ND | 0.13 | 0.07 |
| 3 | Me | H | R⁴ | H | H | 1 | ND | ND | 0.68 | 0.34 |
| 4 | Me | H | Me | Me | H | 1 | 1.6 | 0.58 | ND | ND |
| 5 | Me | H | Me | Me | Me | 1 | 1.0 | 0.40 | ND | ND |
| 6 | Me | Me | Me | H | H | 1 | 1.0 | 0.34 | 0.38 | 0.15 |
| 7^{(c)} | Me | Me | Me | H | H | 2 | NA | ND | ND | ND |
| 8^{(c)} | Me | Me | Me | H | H | 3 | NA | ND | ND | ND |
| 9 | Me | Me | R⁴ | H | H | 1 | ND | ND | 0.49 | 0.20 |
| DMB^{(d)} | — | — | — | — | — | — | ND | ND | — | — |

^{(a)}Annealing to form siRNA duplexes was carried out at room temperature.
^{(b)}Annealing to form siRNA duplexes was carried out at 65° C.
^{(c)}Annealing to form siRNA duplexes was carried out at room temperature, followed by overnight freezing
^{(d)}Negative control: the siRNA containing the same sequences, with the exception that the groups containing disulfides are replaced with 3,3-dimethylbutyl (DMB); DMB is linked irreversibly (under physiological conditions) to phosphate.
ND = not determined.
NA = not active.

EC₅₀ (at 48 h) of the hybridized polynucleotide of the invention (for the structures see FIG. 1B) was measured to be 1.1 nM.

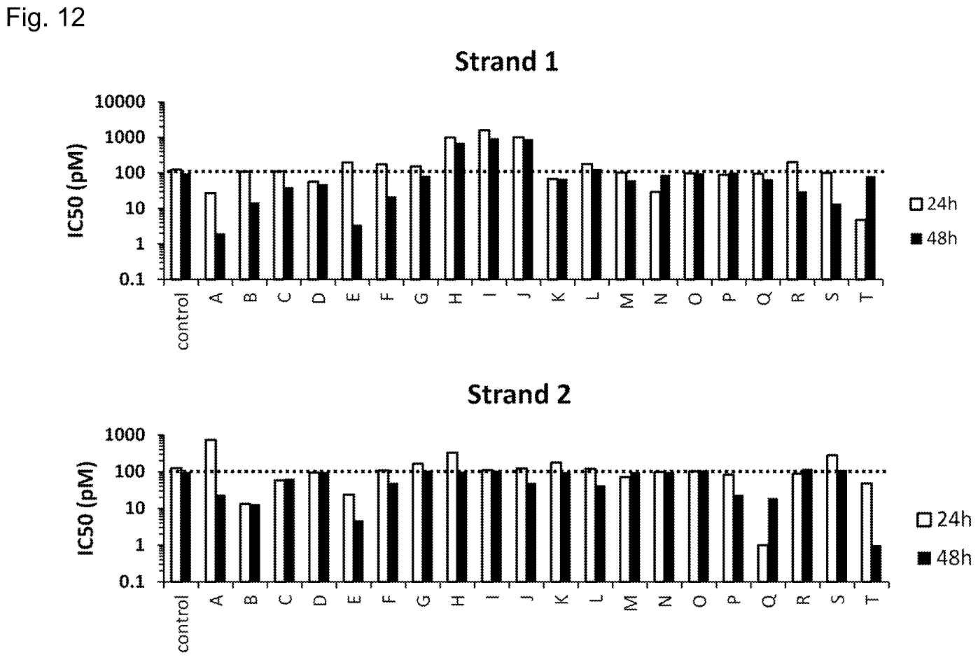

Table 11 shows the data for other hybridized polynucleotides of the invention (for the structures see FIG. 1A), in which certain uridines (labeled with an arrow) have an internucleotide 3′-phosphotriester having the structure shown in Table 11. The in vitro transfection data for siRNA including bioreversible and non-bioreversible group are provided in FIG. 12.

### Transfection Data in SKOV-3-Luc Cells:

TABLE 11
SEQ ID NO: 112: GCUACAUUCUGGAGACAUAUt
SEQ ID NO: 113: tUCGAUGUAAGACCUCUGUAU

| | EC50 (nM) | |
|---|---|---|
| Compound | 24 h | 48 h |
| Control siRNA | 0.01 | 0.01 |
| 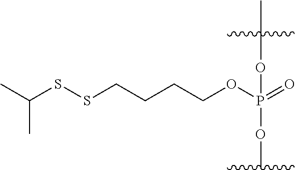 [SMILES: *OP(=O)(O*)OCCCCSSC(C)C] | 0.43 | 0.11 |
| 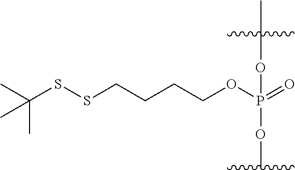 [SMILES: *OP(=O)(O*)OCCCCSSC(C)(C)C] | 0.66 | 0.13 |
| 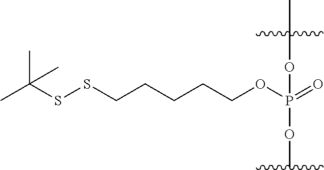 [SMILES: *OP(=O)(O*)OCCCCCSSC(C)(C)C] | >10 | >10 |
| 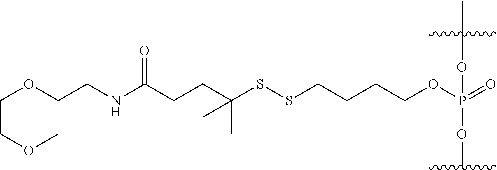 [SMILES: *OP(=O)(O*)OCCCCSSC(C)(C)CCC(=O)NCCOCCOC] | 0.43 | — |
| 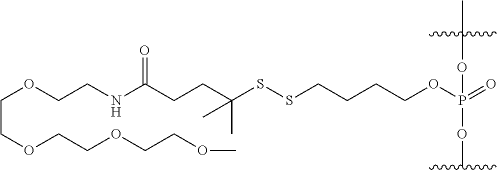 [SMILES: *OP(=O)(O*)OCCCCSSC(C)(C)CCC(=O)NCCOCCOCCOCCOC] | 0.78 | 0.19 |
| 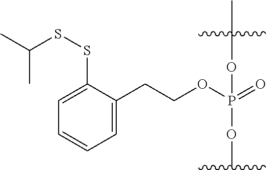 [SMILES: *OP(=O)(O*)OCCc1ccccc1SSC(C)C] | 0.15 | 0.06 |
| 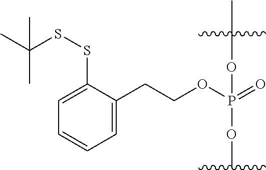 [SMILES: *OP(=O)(O*)OCCc1ccccc1SSC(C)(C)C] | 0.73 | 0.13 |
| 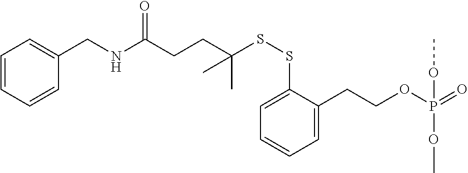 | 0.16 | 0.06 |
| 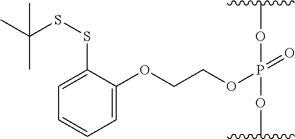 [SMILES: *OP(=O)(O*)OCCOc1ccccc1SSC(C)(C)C] | 1.8 | 0.70 |
| 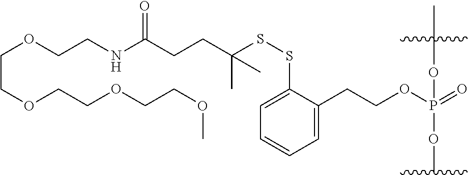 [SMILES: *OP(=O)(O*)OCCc1ccccc1SSC(C)(C)CCC(=O)NCCOCCOCCOCCOC] | 1.01 | 0.42 |
| Control siRNA | 0.01 | 0.01 |
| 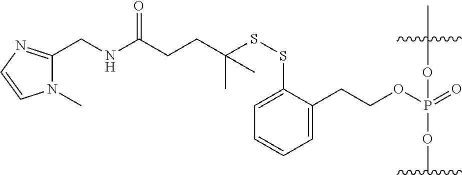 [SMILES: *OP(=O)(O*)OCCc1ccccc1SSC(C)(C)CCC(=O)NCc1nccn1C] | 0.82 | 0.22 |

### Mouse Primary Hepatocyte Isolation and In Vitro Experiments:

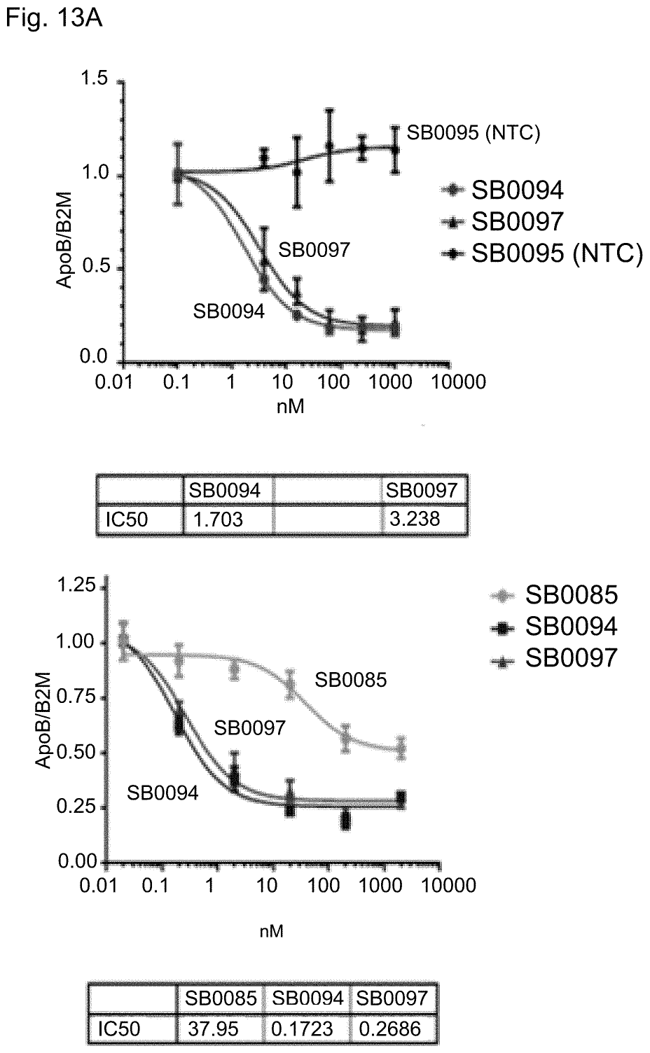

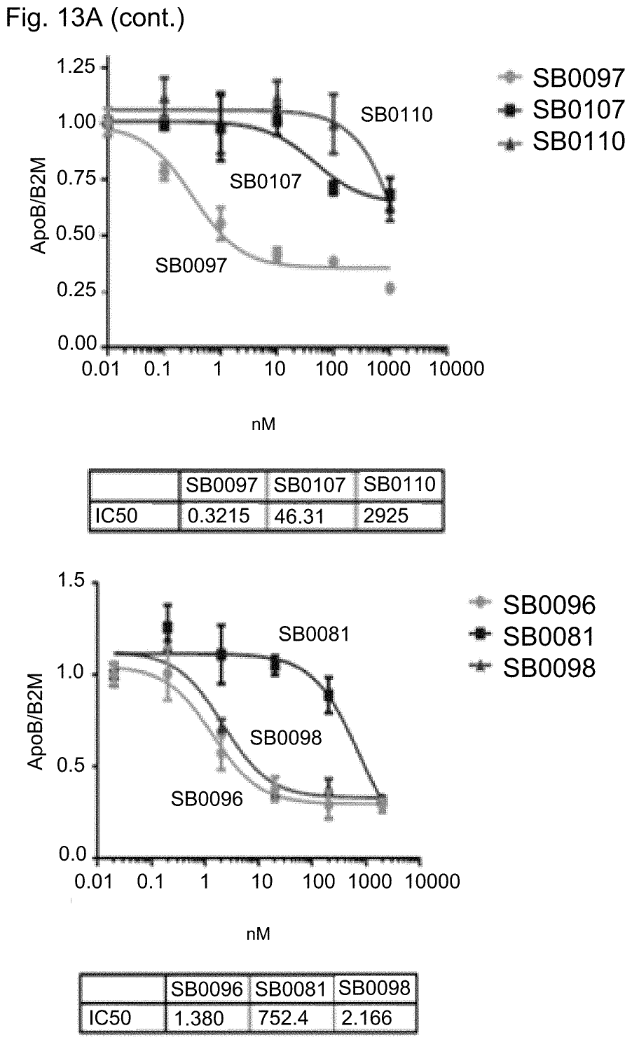

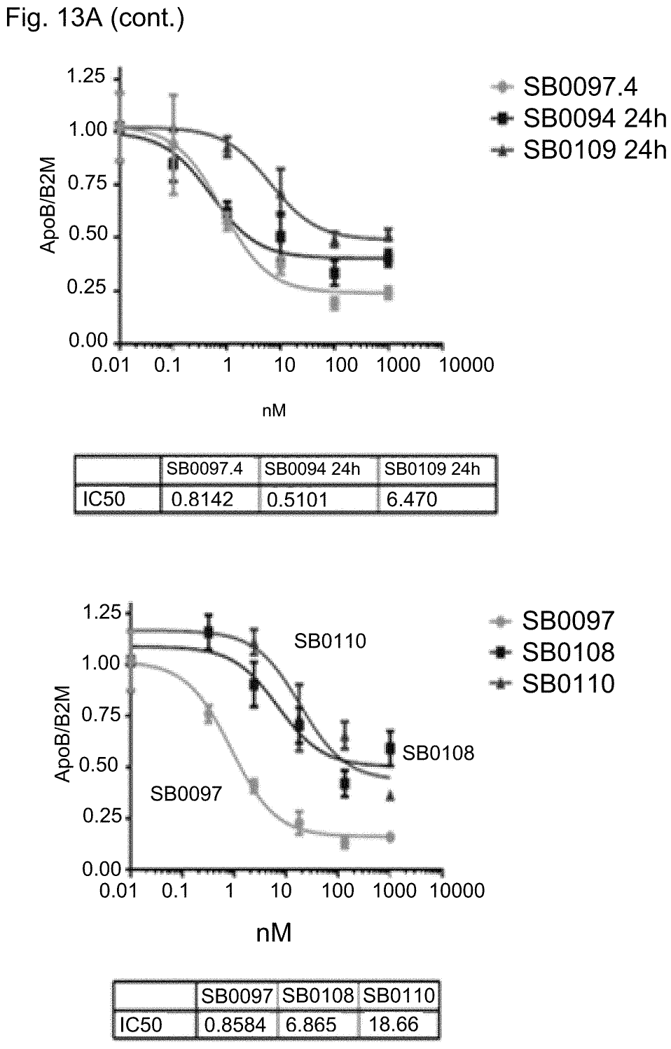

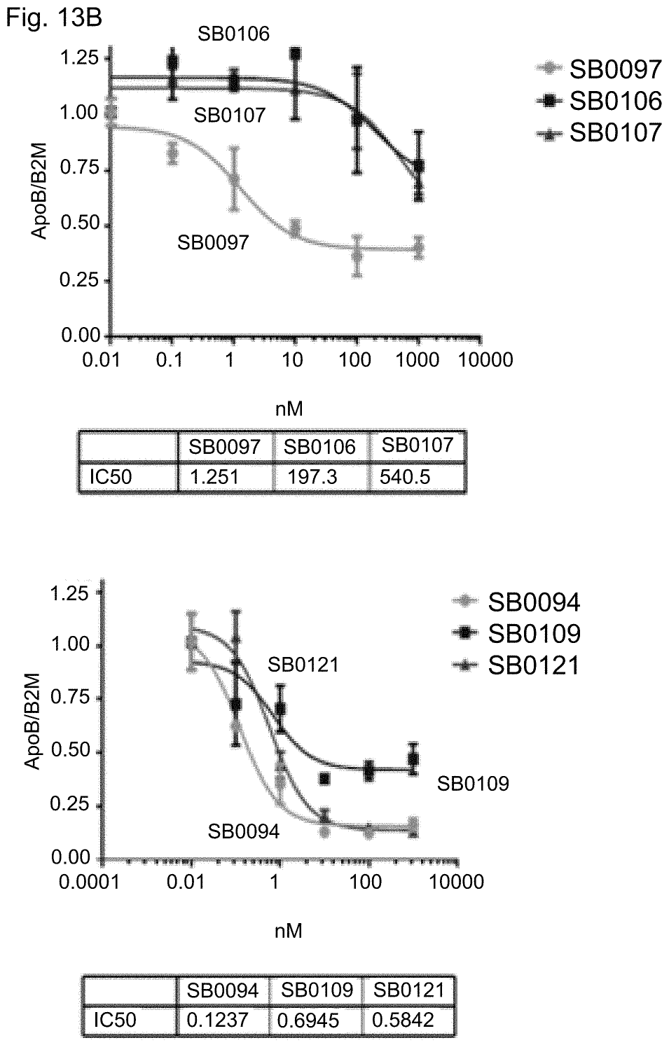

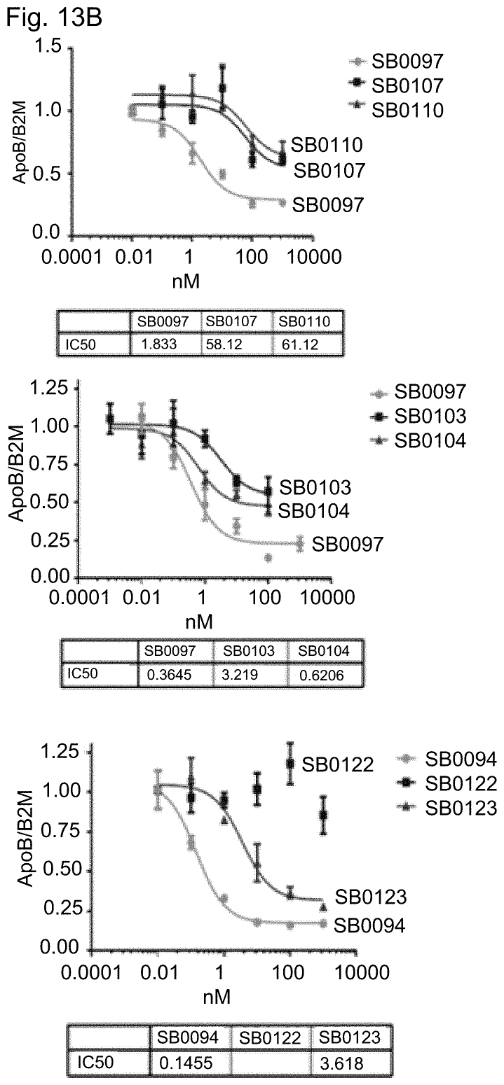

Primary mouse hepatocytes were isolated usingthestandard two-stepcollagenase perfusion technique (Li et al. Methods Mol. Biol., 633:185-196; 2010; the disclosure of which is incorporated herein by reference in its entirety). Briefly, a 6-10 week old female C57/Bl6 mouse was anesthetized by intraperitoneal injection of a mixture of ketamine (80-100 mg/kg)/xylazine (5-10 mg/kg). The abdominal cavity was then exposed, and the visceral vena cava was cannulated using a 22 G needle. The hepatic vein was severed, and the liver was immediately perfused for 5-10 min using a solution of phosphate-buffered saline (PBS) containing 0.5 mM ETDA. This solution was immediately switched to a solution of collagenase (100 IU/ml) in Dulbecco's Modified Eagle's Medium (DMEM, Gibco) for another 5-10 min. At the end of perfusion, the liver was removed and the hepatocytes were collected in DMEM containing 10% fetal bovine serum at 4° C. The cells were then filtered through a 70 μm sterile filter, washed three times in the same solution, and cell viability was assessed using Trypan Blue staining. Cells were then seeded in 96-well plates coated with 0.1% rat tail collagen or 2% matrigel and incubated for 3-4 hours at 37° C. in a 5% CO₂ incubator. Test compounds were then added to cells and incubated at 37° C. in a 5% CO₂ incubator. At the end of the incubation period, the cells were lysed, the mRNA was isolated and the expression of the target gene was measured by qPCR and normalized to a house-keeping gene using standard protocols. The results are graphed in FIGS. 13A and 13B and are provided in Table 12.

TABLE 12

| | Activity in Primary Mouse Hepatocytes | | |
|---|---|---|---|
| Compound | ApoB | GAPDH | ATIII |
| # | IC₅₀ (nM) | IC₅₀ (nM) | IC₅₀ (nM) |
| SB0129 | 0.1 | | |
| SB0130 | 2 | | |
| SB0134 | 1 | | |
| SB0141 | 6 | | |
| SB0142 | 2.5 | | |
| SB0146 | | 0.2 | |
| SB0147 | | 1 | |
| SB0148 | | 0.05 | |
| SB0150 | 172 | | |
| SB0154 | 0.1 | | |
| SB0155 | 0.2 | | |
| SB0156 | 0.5 | | |
| SB0157 | 0.2 | | |
| SB0162 | 1 | | |
| SB0163 | 2.5 | | |
| SB0164 | 1.5 | | |
| SB0234 | 0.5 | | |
| SB0235 | 0.6 | | |
| SB0236 | 3.2 | | |
| SB0243 | 0.3 | | |
| SB0244 | 1.4 | | |
| SB0245 | 5.8 | | |
| SB0246 | 5.6 | | |
| SB0206 | | | 0.006 |
| SB0211 | | | 0.2 |
| SB0254 | | | 1.1 |
| SB0255 | | | 0.3 |
| SB0256 | | | 0.01 |
| SB0257 | | | 0.007 |
| SB0258 | | | 0.006 |
| SB0296 | | | 0.1 |
| SB0319 | | | 1 |
| SB0320 | | | 0.85 |
| SB0321 | | | 0.006 |
| SB0322 | | | 0.09 |
| SB0323 | | | 1.3 |
| SB0332 | | | 0.005 |
| SB0333 | | | 0.06 |
| SB0334 | | | 0.004 |
| SB0335 | | | 0.03 |
| SB0336 | | | 0.04 |
| SB0345 | | | 1.1 |
| SB0346 | | | 0.08 |
| SB0381 | | | 0.45 |
| SB0516 | | | 4.7 |
| SB0517 | | | 1 |
| SB0518 | | | 0.64 |
| SB0535 | | | 1.5 |
| SB0538 | | | 0.16 |
| SB0539 | | | 12.4 |
| SB0590 | | | 0.17 |
| SB0591 | | | 0.14 |
| SB0592 | | | 0.23 |
| SB0593 | | | 0.16 |
| SB0594 | | | 0.12 |
| SB0595 | | | 0.45 |
| SB0596 | | | 0.32 |
| SB0597 | | | 0.17 |
| SB0598 | | | 0.21 |
| SB0599 | | | 0.07 |
| SB0600 | | | 1.2 |
| SB0609 | | | 2.3 |
| SB0610 | | | 0.28 |
| SB0614 | | | 0.4 |
| SB0616 | | | 0.33 |
| SB0617 | | | 0.24 |
| SB0619 | | | 0.4 |
| SB0620 | | | 0.28 |
| SB0621 | | | 0.23 |
| SB0622 | | | 0.18 |
| SB0639 | | | 3.5 |
| SB0640 | | | 1.6 |
| SB0641 | | | 0.11 |
| SB0642 | | | 0.1 |
| SB0643 | | | 0.27 |
| SB0665 | | | 1.72 |
| SB0666 | | | 0.1 |
| SB0672 | | | 3.2 |
| SB0673 | | | 2.2 |
| SB0674 | | | 0.56 |
| SB0675 | | | 1.4 |
| SB0676 | | | 0.14 |
| SB0677 | | | 0.22 |
| SB0678 | | | 0.05 |
| SB0708 | | | >100 |
| SB0709 | | | >100 |
| SB0721 | | | 0.29 |
| SB0722 | | | 0.3 |
| SB0723 | | | 0.38 |
| SB0724 | | | 0.36 |

### Example 3. Cell Binding Experiments

Disulfide Phosphotriester Oligonucleotide-Cy3 Cell Binding General Protocol: polynucleotide constructs of the invention containing disulfide bioreversible groups were annealed to G^{2′Mod}Cy3 (guide strand) at a final concentration of 10 mM.

Cell treatment setup: 40,000 cells were plated per well in a 48 well plate; cells were allowed to adhere overnight. Then, cells were washed once with 500 μl of PBS then 150 μL treatments were added (Note: for free folic acid samples, cells were treated with media containing 2.3 mM folic acid for 1 h prior to treatment). Cells were treated for 4 h; after 4 h, cells were washed once with PBS, trypsinized, and analyzed by flow cytometry for siRNA-Cy3 cell association.

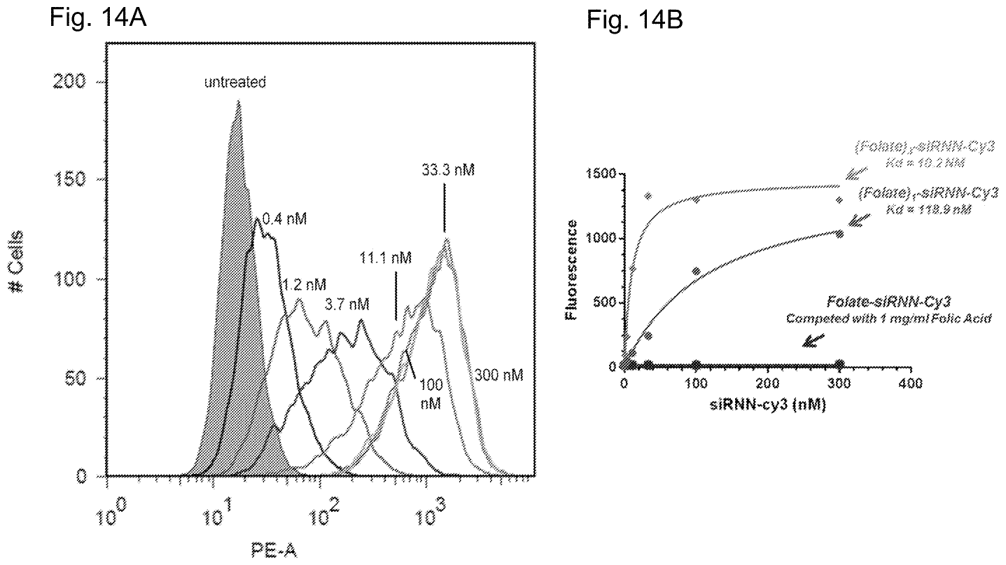

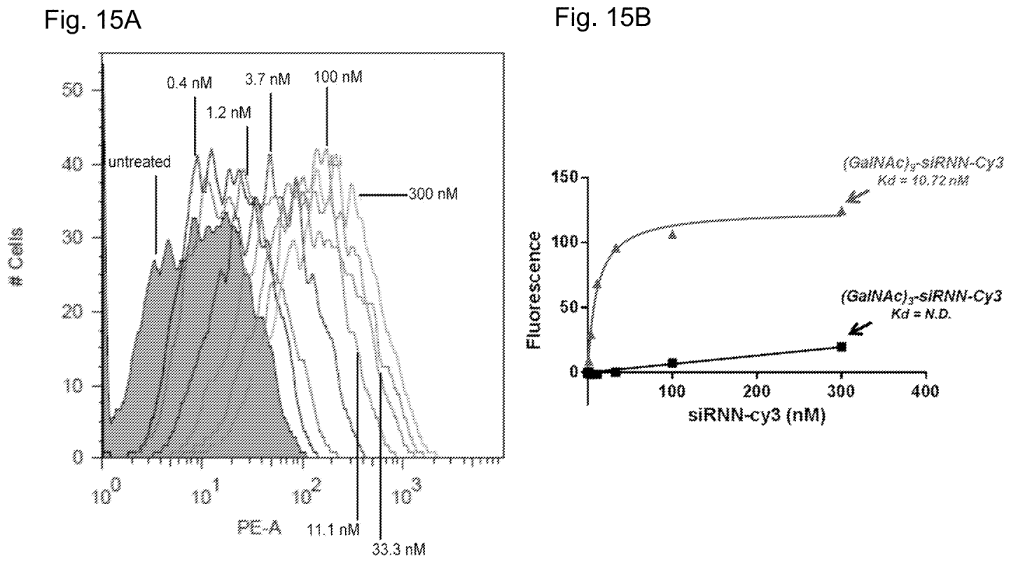

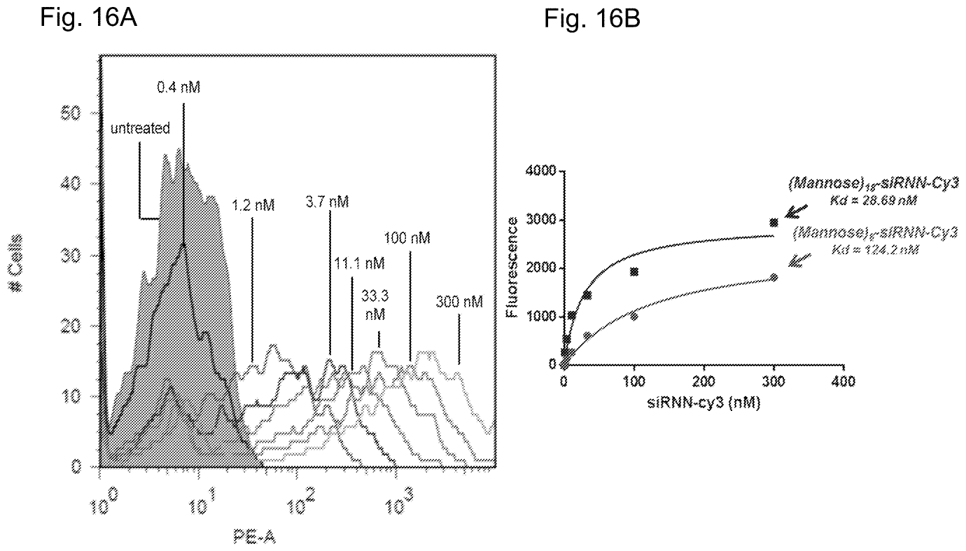

Results of these experiments are shown in FIGS. 14A, 14B, 15A, 15B, 16A, and 16B. FIG. 14A shows dose curves for (Folate)₃-siRNN-Cy3 conjugate binding to KB cell. FIG. 14B shows a graph determining dissociation constants (K_{d}) for (Folate)₃-siRNN-Cy3 and (Folate)₁-siRNN-Cy3 conjugates. FIG. 15A shows dose curves for (GaINAc)₉-siRNN-Cy3 conjugate binding to HepG2 cells. FIG. 15B shows a graph determining dissociation constants (K_{d}) for (GaINAc)₉-siRNN-Cy3 and (GaINAc)₃-siRNN-Cy3 conjugates. FIG. 16A shows dose curves for (Mannose)₁₈-siRNN-Cy3 conjugate binding to primary peritoneal macrophages. FIG. 16B shows a graph determining dissociation constants (K_{d}) for (Mannose)₁₈-siRNN-Cy3 and (Mannose)₆-siRNN-Cy3 conjugates.

### Example 4. In Vivo Activity Assays

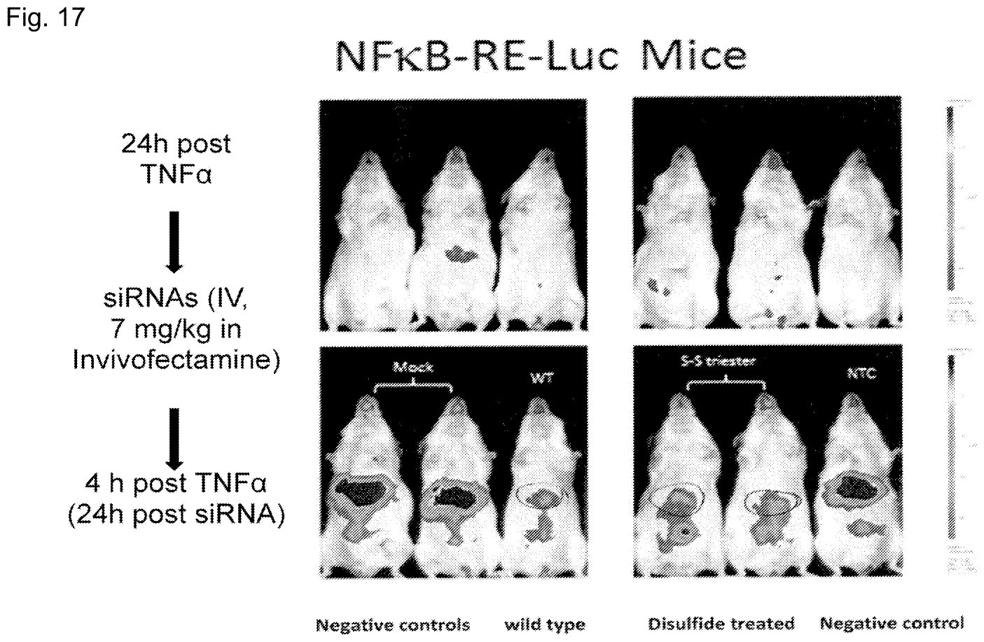

The in vivo activity of a luciferase disulfide phosphotriester molecule was tested using male NFKB-RE-Luc mice (Taconic). These mice express the luciferase gene (GL3) throughout the body, including the liver, and the luciferase activity is inducible by NFKB activators such as TNFα. Test agents (luciferase disulfide phosphotriester, wild-type luciferase siRNA sequence, and a non-targeting control siRNA sequence) were complexed with Invivofectamine 2.0 Reagent (Life Technologies) according to the manufacturer's recommendations and injected (˜200 μL, 7 mg/kg body weight) into the tail vein using sterile insulin syringes (n=1-2 mice/treatment). Two additional mice were injected with the same volume of vehicle and served as a mock treatment control. Twenty-four hours post injection, mice were subjected to intraperitoneal injection of murine TNFα (0.03 μg/g) to induce liver luciferase activity. Four hours after TNFα injection, mice were injected D-luciferin (150 mg/kg) intraperitoneally, and liver luciferase activity was measured using the IVIS Lumina whole body imager (Perkin Elmer) approximately 10 minutes after D-luciferin injection. Mice were imaged again 3, 6, and 8 days after siRNA administration to assess liver luciferase activity as described above. Results of this assay are shown in FIG. 17.

### In Vivo Experiments:

Test compounds were administered to female C57Bl6 mice via either subcutaneous or intravenous (lateral tail vein) injection (200 μL; 3 mice/treatment). At the appropriate time point post injection, mice were sacrificed and blood samples were collected by cardiac puncture. Approximately 50-100 mg piece of liver sample was collected and was immediately frozen in liquid nitrogen. Total mRNA was isolated from liver homogenates using standard protocols and the expression of target gene was quantitated by qPCR and normalized to a house-keeping gene using standard protocols.

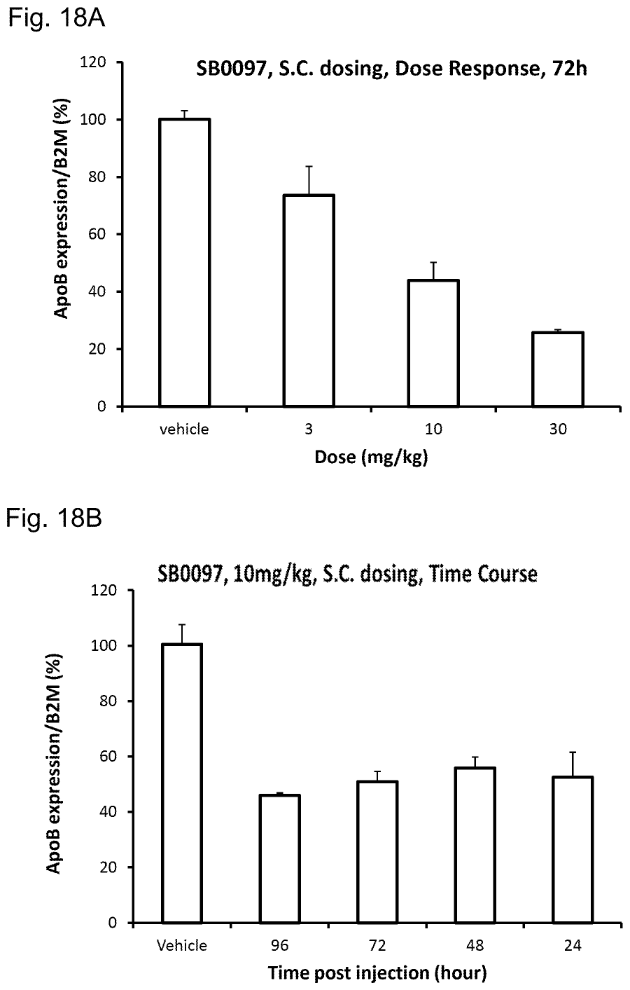

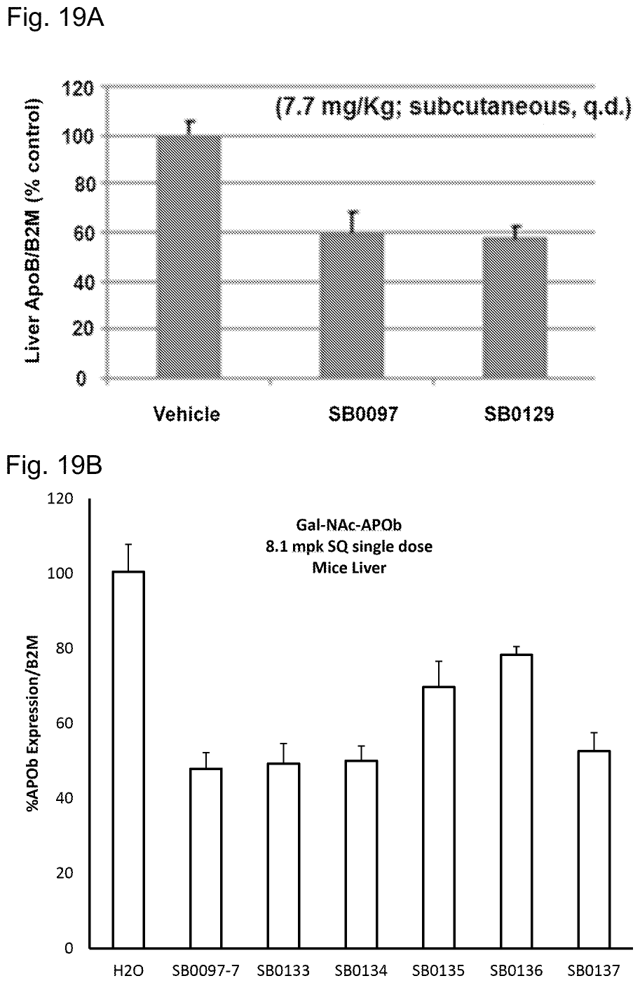

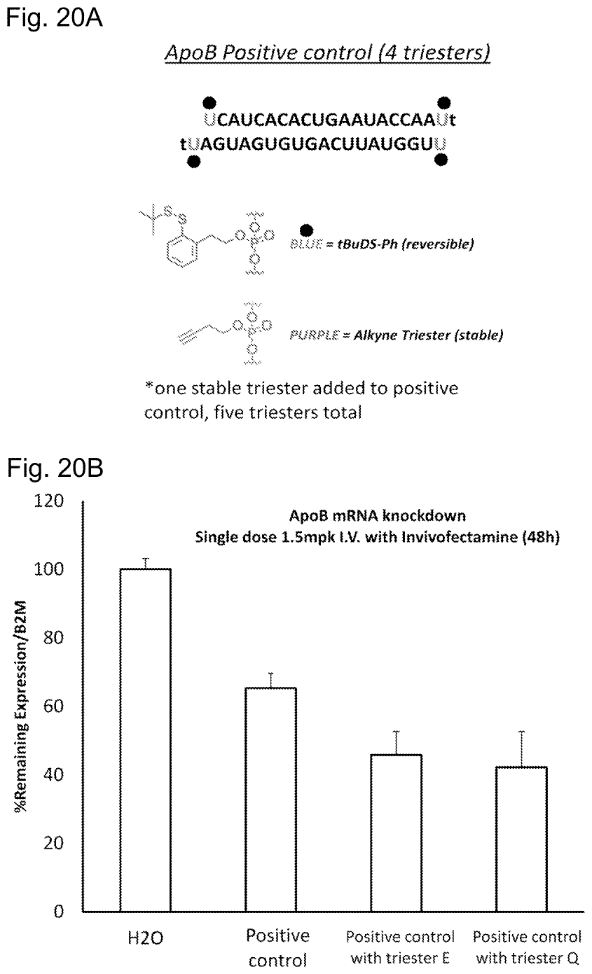

The results are shown in FIGS. 18A, 18B, 19A, 19B, and 20B (for the siRNA structures used to generate the data in FIG. 20B, see FIG. 20A).

For an exemplary procedure for isolation and culture of mouse hepatocytes, see: Li et al., Methods Mol. Biol., 633:185-196; 2010; the disclosure of which is incorporated herein by reference in its entirety.

### Pharmacology:

TABLE 13
% ApoB mRNA Remaining*

| | S.C. dosing | | I.V. dosing | |
|---|---|---|---|---|
| Compound | (mg/kg) | % | (mg/kg) | % |
| # | Mean | SEM | Mean | SEM |
| SB-0081 | 48.7 (30) | 3.8 | | |
| SB-0085 | 114 (30) | 8.6 | | |
| SB-0094 | 27.7 (30) | 0.8 | 32.5 (20) | 4.6 |
| SB-0094 | 63.2 (10) | 2.2 | | |
| SB-0095 | 91.8 (30) | 7 | | |
| SB-0096 | 25.0 (30) | 4.1 | | |
| SB-0097 | 22.3 (30) | 4.3 | 44.8 (20) | 2.8 |
| SB-0097 | 49.5 (10) | 1.3 | | |
| SB-0098 | 17.3 (30) | 2.6 | | |
| SB-0102 | 73.5 (15) | 3.9 | | |
| SB-0106 | 84.1 (10) | 6 | | |
| SB-0107 | 68.2 (10) | 3.8 | 53.3 (30) | 3.2 |
| SB-0108 | 78.5 (10) | 9.5 | | |
| SB-0109 | 97.8 (12.5) | 12.5 | 91.2 (20) | 11.9 |
| SB-0121 | 75.5 (10) | 1.5 | | |
| SB-0122 | 92.3 (20) | 6.5 | | |
| SB-0123 | 58.6 (20) | 8.2 | | |
| SB-0129 | 24.7 (7) | 5.4 | | |
| SB-0130 | | | 56.5 (4.3) | 6 |
| SB-0141 | | | 42.8 (4.3) | 7.8 |
| SB-0162 | | | 40.3 (10) | 1.5 |
| SB-0222 | 36.5 (10) | 9.3 | | |
| SB-0223 | 38 (10) | 1.6 | | |
| SB-0224 | 36.4 (10) | 5.2 | | |
| SB-0225 | 39.9 (10) | 1 | | |
| SB-0226 | 34.6 (10) | 2.2 | | |
| SB-0234 | | | 40.9 (10) | 3 |
| SB-0235 | | | 45.4 (10) | 3.3 |
| SB-0236 | | | 45.0 (10) | 5.5 |
| SB-0243 | 39.1 (7) | 3.2 | | |
| SB-0244 | 36.7 (7) | 0.8 | | |
| SB-0245 | 54.4 (7) | 2.9 | | |
| SB-0246 | 48.8 (7) | 2.4 | | |

*Gene expression was measured 48-72 h post dose

TABLE 14
Remaining AT3 Plasma Activity*

| | plasma activity | | Dose |
|---|---|---|---|
| Compound # | Mean | SEM | (mg/kg, S.C.) |
| SB-0206 | 35.3 | 2.7 | 0.5 |
| SB-0255 | 93.2 | 9.5 | 2 |
| SB-0256 | 71.2 | 3.3 | 0.75 |
| SB-0257 | 54.7 | 4.6 | 0.75 |
| SB-0258 | 94.2 | 4.5 | 0.75 |
| SB-0295 | 16.3 | 6 | 2 |
| SB-0296 | 19.6 | 2 | 1 |
| SB-0297 | 19.1 | 1.1 | 2 |
| SB-0319 | 68.6 | 7.7 | 1 |
| SB-0320 | 68.7 | 2.3 | 1 |
| SB-0321 | 30.2 | 1.7 | 1 |
| SB-0322 | 40.7 | 5.9 | 1 |
| SB-0323 | 80.8 | 3.6 | 1 |
| SB-0332 | 41.4 | 5.3 | 1 |
| SB-0333 | 23.3 | 2.5 | 1 |
| SB-0334 | 23 | 0.9 | 1 |
| SB-0335 | 55.6 | 7.1 | 1 |
| SB-0336 | 55 | 2.9 | 1 |
| SB-0337 | 54.5 | 3.6 | 1 |
| SB-0338 | 70.3 | 2 | 1 |
| SB-0339 | 53.7 | 4.2 | 1 |
| SB-0340 | 32.2 | 5.4 | 1 |
| SB-0345 | 24.1 | 2 | 1 |
| SB-0347 | 62.5 | 7.2 | 1 |
| SB-0348 | 20.3 | 1.6 | 1 |
| SB-0349 | 29.7 | 0.9 | 1 |
| SB-0366 | 20.2 | 3.9 | 1 |
| SB-0367 | 20.3 | 5.1 | 1 |
| SB-0368 | 38.9 | 3.3 | 1 |
| SB-0369 | 34.8 | 2.5 | 1 |
| SB-0370 | 21.3 | 1.2 | 1 |
| SB-0371 | 22.9 | 5.2 | 1 |
| SB-0372 | 8.7 | 0.8 | 1 |
| SB-0373 | 11.7 | 2.8 | 1 |
| SB-0374 | 8.3 | 0.8 | 1 |
| SB-0375 | 10.2 | 1.8 | 1 |
| SB-0376 | 30.7 | 2.9 | 1 |
| SB-0377 | 22.5 | 7.4 | 1 |
| SB-0378 | 25.6 | 1.4 | 1 |
| SB-0379 | 30.9 | 1.3 | 1 |
| SB-0381 | 34.9 | 2.7 | 1 |
| SB-0382 | 43.9 | 7.5 | 1 |
| SB-0459 | 36.3 | 2 | 0.5 |
| SB-0460 | 48.5 | 6.3 | 0.5 |
| SB-0461 | 45 | 0.9 | 0.5 |
| SB-0462 | 54.2 | 1.3 | 0.5 |
| SB-0463 | 51 | 3.4 | 0.5 |
| SB-0464 | 53.5 | 3.2 | 0.5 |
| SB-0481 | 96.2 | 5.9 | 0.5 |
| SB-0482 | 111.5 | 2.7 | 0.5 |
| SB-0483 | 103.2 | 8 | 0.5 |
| SB-0484 | 114.5 | 5.1 | 0.5 |
| SB-0506 | 40.2 | 4.1 | 0.5 |
| SB-0507 | 56.6 | 8 | 0.5 |
| SB-0508 | 51.9 | 3.4 | 0.5 |
| SB-0509 | 47.3 | 1.8 | 0.5 |
| SB-0510 | 46.8 | 1.3 | 0.5 |
| SB-0511 | 48.2 | 2.3 | 0.5 |
| SB-0512 | 53 | 1.5 | 0.5 |
| SB-0516 | 47.8 | 3.8 | 0.5 |
| SB-0517 | 48.8 | 2.2 | 0.5 |
| SB-0518 | 57.1 | 4.5 | 0.5 |
| SB-0522 | 47.7 | 1.5 | 0.5 |
| SB-0523 | 38.6 | 1.6 | 0.5 |
| SB-0524 | 48.2 | 9.2 | 0.5 |
| SB-0525 | 39.4 | 3.6 | 0.5 |
| SB-0526 | 38.4 | 3.5 | 0.5 |
| SB-0527 | 40.5 | 3.1 | 0.5 |
| SB-0528 | 82 | 2.5 | 0.5 |
| SB-0529 | 47.3 | 4.7 | 0.5 |
| SB-0530 | 41.9 | 6.4 | 0.5 |
| SB-0531 | 46.6 | 4.1 | 0.5 |
| SB-0532 | 43.7 | 3.5 | 0.5 |
| SB-0533 | 46.7 | 4.3 | 0.5 |
| SB-0535 | 103.3 | 2 | 0.5 |
| SB-0538 | 52.2 | 5.5 | 0.6 |
| SB-0539 | 95.4 | 3.8 | 0.6 |
| SB-0560 | 50.2 | 4.6 | 0.5 |
| SB-0561 | 76.5 | 3 | 0.5 |
| SB-0588 | 78.5 | 2.1 | 0.5 |
| SB-0589 | 88 | 1.3 | 0.5 |
| SB-0590 | 48.4 | 4.5 | 0.5 |
| SB-0591 | 37.4 | 2.7 | 0.5 |
| SB-0592 | 44.9 | 1.7 | 0.5 |
| SB-0593 | 51 | 0.6 | 0.5 |
| SB-0594 | 39.3 | 0.8 | 0.5 |
| SB-0595 | 44.6 | 2.7 | 0.5 |
| SB-0596 | 22.2 | 0.4 | 0.5 |
| SB-0597 | 41.1 | 3.7 | 0.5 |
| SB-0598 | 71.5 | 1.1 | 0.5 |
| SB-0599 | 47.3 | 4.8 | 0.5 |
| SB-0600 | 76 | 1.5 | 0.5 |
| SB-0609 | 89.2 | 6.3 | 0.5 |
| SB-0610 | 96.3 | 0.9 | 0.5 |
| SB-0614 | 54 | 5.8 | 0.5 |
| SB-0616 | 56.7 | 6.8 | 0.5 |
| SB-0617 | 54.6 | 4 | 0.5 |
| SB-0618 | 67.8 | 2.5 | 0.5 |
| SB-0619 | 38.4 | 2.9 | 0.5 |
| SB-0620 | 44.4 | 0.7 | 0.5 |
| SB-0621 | 40.8 | 3.3 | 0.5 |
| SB-0622 | 42.1 | 5.5 | 0.5 |
| SB-0627 | 79.6 | 5.2 | 0.5 |
| SB-0639 | 89.2 | 3.3 | 0.4 |
| SB-0640 | 89.5 | 2.4 | 0.4 |
| SB-0641 | 32.6 | 3.7 | 0.5 |
| SB-0642 | 42 | 1.5 | 0.5 |
| SB-0643 | 65.2 | 2.3 | 0.5 |
| SB-0665 | 52.7 | 6.8 | 0.5 |
| SB-0666 | 41.2 | 2 | 0.5 |
| SB-0672 | 95.8 | 7.7 | 0.5 |
| SB-0673 | 99.5 | 1.4 | 0.5 |
| SB-0674 | 97.2 | 5.9 | 0.5 |
| SB-0675 | 98.5 | 5.8 | 0.5 |
| SB-0676 | 95.6 | 3.3 | 0.5 |
| SB-0677 | 93.5 | 5.5 | 0.5 |
| SB-0678 | 86.4 | 2.5 | 0.5 |
| SB-0706 | 96.3 | 8.2 | 0.5 |
| SB-0707 | 56 | 12 | 0.5 |
| SB-0720 | 56.2 | 5.7 | 0.5 |
| SB-0721 | 60.1 | 4.5 | 0.5 |
| SB-0722 | 50 | 3.8 | 0.5 |
| SB-0723 | 72.5 | 3.3 | 0.5 |
| SB-0724 | 59.9 | 5.3 | 0.5 |
| SB-0730 | 97 | 5.5 | 0.5 |
| SB-0731 | 99.7 | 3.3 | 0.5 |
| SB-0732 | 110.5 | 3 | 0.5 |
| SB-0733 | 101.5 | 3.9 | 0.5 |
| SB-0734 | 103.3 | 4.9 | 0.5 |

*AT3 activity was measured on day 7-10 post dose

### GAPDH-Mannose Conjugates Demonstrate Dose-Dependent In Vivo Activity

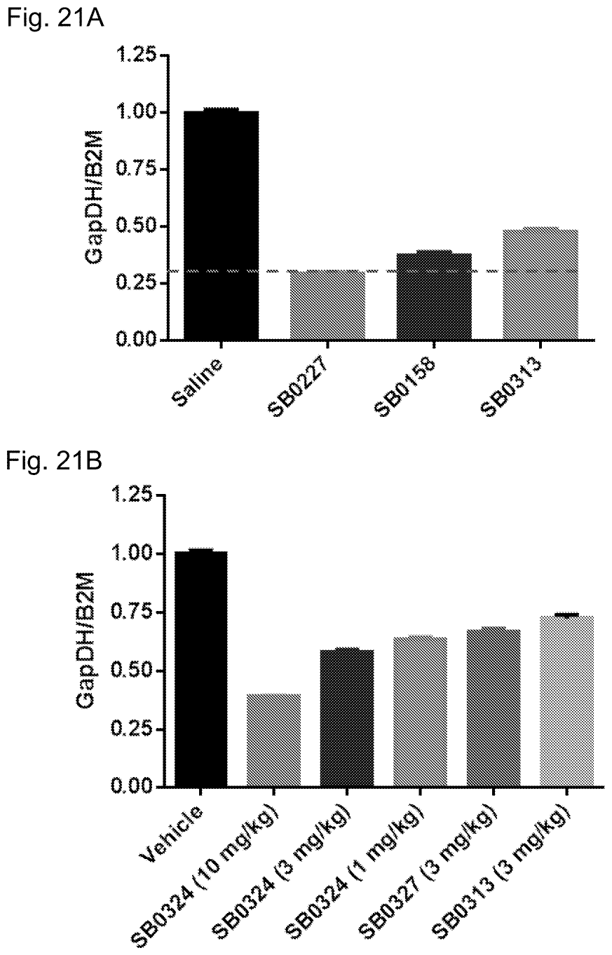

Protocol 1: Female C57Bl6 mice received an intra-peritoneal (IP) injection of 3% thioglycollate (2.5 mL). Test compounds (10 mg/kg) were administered via IP injection 6 h, 24 h, and 48 h post thioglycollate injection (three doses). The peritoneal macrophages were harvested 24 h later by washing the peritoneal cavity with ice-cold PBS. Cells were washed twice with PBS, re-suspended in RPMI containing 10% fetal calf serum and plated in 96-well plates for 3 h to allow macrophage adherence. Cells were then washed, lysed, and total mRNA was extracted using standard methods. The expression of GAPDH gene was quantitated by RTqPCR and normalized to a house-keeping gene. The results are provided in FIG. 21A.

Protocol 2: Female C57Bl6 mice received an intra-peritoneal (IP) injection of 3% thioglycollate (2.5 mL. Test compounds were administered via IP injection 6 hand 24 h post thioglycollate injection (2 doses). The peritoneal macrophages were harvested 24 h later by washing the peritoneal cavity with ice-cold PBS. Cells were washed twice with PBS, re-suspended in RPMI containing 10% fetal calf serum and plated in 96-well plates for 3 h at 37° C., under 5% CO₂ atmosphere to allow macrophage adherence. Cells were then washed to remove non-macrophage cells, lysed, and total mRNA extracted using standard methods. The expression of GAPDH gene was quantitated by RTqPCR and normalized to a house-keeping gene. The results are provided in FIG. 21B.

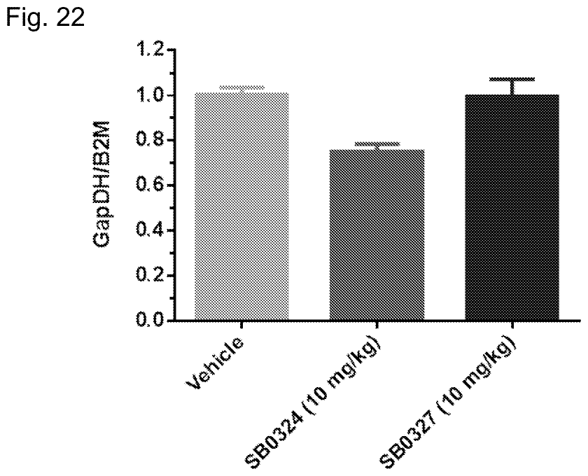

Protocol 3: Female C57Bl6 mice received an intra-peritoneal (IP) injection of 3% thioglycollate (2.5 mL). Test compounds were administered via IP injection 24 h post thioglycollate injection (single dose). The peritoneal macrophages were harvested 2 h later by washing the peritoneal cavity with ice-cold PBS. Cells were washed twice with PBS, re-suspended in RPMI containing 10% fetal calf serum and plated in 96-well plates for 3 h at 37° C., under 5% CO₂ atmosphere to allow macrophage adherence. Non-macrophage cells were washed away by PBS, and macrophages were incubated in RPMI containing 10% fetal bovine serum for 48 h at 37° C., under 5% CO₂ atmosphere. Cells were then lysed, and GAPDH gene expression was quantitated by RTqPCR and normalized to a house-keeping gene. The results are provided in FIG. 22.

### Example 5: Mouse Primary Bone Marrow Progenitor Cells Isolation and In Vitro Experiments Using Macrophages

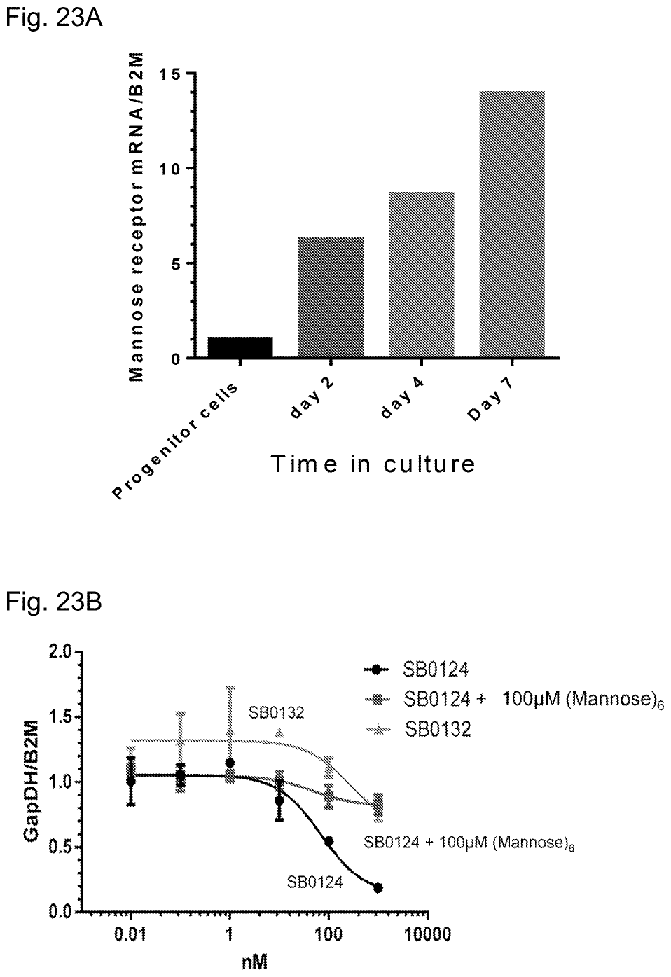

Protocol 1: Mouse primary bone marrow progenitor cells were isolated from the femurs and tibias of female C57Bl6 mice according to published protocols. Cells were immediately washed with PBS at 4° C. and suspended at 2×10⁶ cells/ml in RPMI containing 10% fetal calf serum and 20 ng/ml recombinant mouse M-CSF. Cells were seeded in 96-well plates and incubated for 7 days at 37° C., under 5% CO₂ atmosphere to allow differentiation to macrophages. Cells were washed every 24 hrs to remove potential non-macrophage cells contamination. Cells were used on day 7 based on mannose receptor expression. Mannose receptor expression overtime is graphed in FIG. 23A. Test compounds from Tables 5 and 7 were diluted in serum-free optiMEM and incubated with cells for 48 h. Cells were then lysed, total mRNA extracted and the expression of GAPDH gene was quantitated using RTqPCR and normalized to a house-keeping gene. Results are shown in FIG. 23B.

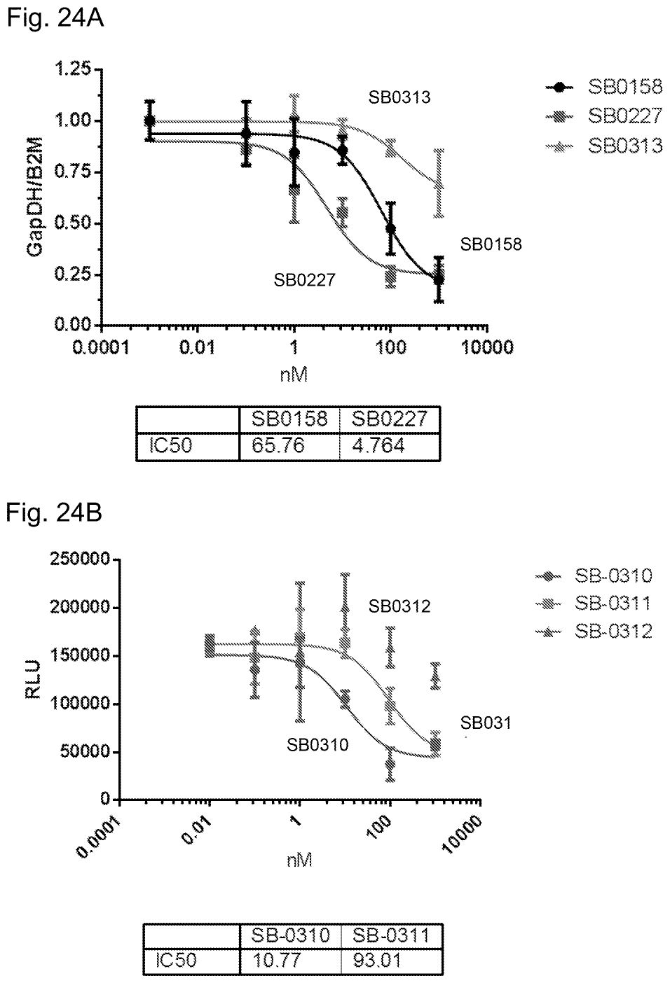

Protocol 2: Mouse primary bone marrow progenitor cells were isolated from the femurs and tibias of female C57Bl6 mice according to published protocols. Cells were immediately washed with PBS at 4° C. and suspended at 2×10⁶ cells/mL in RPMI containing 10% fetal calf serum and 20 ng/mL recombinant mouse CSF. Cells were seeded in 96-well plates and incubated for 3 days at 37° C., under 5% CO₂ atmosphere to allow differentiation to macrophages. On day 4, recombinant mouse IL-4 (20 ng/mL) was added, and cells were incubated for an additional 48 h at 37° C., under 5% CO₂ atmosphere. Test compounds were diluted in OptiMEM and incubated with cells for 48 h. Cells were then lysed, total mRNA was extracted, and the expression of GAPDH gene was quantitated by RTqPCR and normalized to a house-keeping gene. Results are shown in FIG. 24A.

Protocol 3: Mouse primary bone marrow progenitor cells were isolated from the femurs and tibias of b-actin-luc mice (FVB/NTac-Tg-Actb-uc-46Xen, Taconic) according to published protocols. Cells were immediately washed with PBS at 4° C. and suspended at 2×10⁶ cells/ml in RPMI containing 10% fetal calf serum and 20 ng/mL recombinant mouse CSF. Cells were seeded in 96-well plates and incubated for 3 days at 37° C., under 5% CO₂ atmosphere to allow differentiation to macrophages. On day 4, recombinant mouse IL-4 (20 ng/mL) was added, and cells were incubated for an additional 48 h at 37° C., under 5% CO₂ atmosphere. Test compounds were diluted in OptiMEM and incubated with cells for 48 h. Luciferase activity was assessed by the addition of Britelite™ (Perkin Elmer). Results are shown in FIG. 24B.

### Example 6: Mouse Serum Stability

Assessment of serum stability of triester containing oligonucleotides (single and double-strand) was carried out as described below.

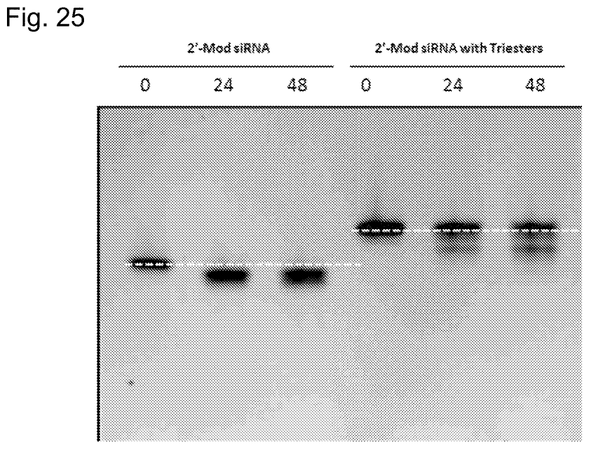

Protocol: 20 μL of 250 μM dsRNA stocks were made up; 4 μL from each were removed and placed in 16 μL of fresh mouse serum; 20 μL samples were placed in PCR plates and heated on thermocycler at 37° C.; 2 μL were removed at indicated time points, added to 18 μL of formamide loading buffer and frozen prior to gel analysis; 2 μL were loaded per well for analysis by gel electrophoresis (15% denaturing gel; ethidium bromide stain). The results are shown in FIG. 25.

### Other Embodiments

Various modifications and variations of the described invention and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A hybridized polynucleotide construct comprising a passenger strand, a guide strand loadable into a RISC complex, and
(i) a 3′-terminal or an internucleotide non-bioreversible group in said guide strand; or
(ii) a 5′-terminal, a 3′-terminal, or an internucleotide non-bioreversible group in said passenger strand, and a 5′-terminal, a 3′-terminal, or an internucleotide disulfide bioreversible group in said guide strand or said passenger strand.

2. The hybridized polynucleotide construct of claim 1, comprising said disulfide bioreversible group, wherein said disulfide bioreversible group comprises -S-S-(Link A)-B,
wherein
Link A is a divalent or a trivalent linker comprising an sp³-hybridized carbon atom bonded to B and a carbon atom bonded to -S-S-, wherein, when Link A is a trivalent linker, the third valency of Link A combines with -S-S- to form optionally substituted C₃₋₉ heterocyclylene, and
B is a 5′-terminal phosphorus (V) group, a 3′-terminal phosphorus (V) group, or an internucleotide phosphorus (V) group.

3. A hybridized polynucleotide construct comprising a passenger strand and a guide strand loadable into a RISC complex, wherein each of said passenger strand and said guide strand has the structure according to the following formula:
5′-D-(Nuc-E)ₙ-Nuc-F, or a salt thereof,
wherein
each n is independently an integer from 10 to 150,
each Nuc is independently a nucleoside; and
D of said guide strand is hydroxyl, phosphate, or a disulfide bioreversible group;
D of said passenger strand is H, hydroxyl, optionally substituted C₁₋₆ alkoxy, a protected hydroxyl group, phosphate, diphosphate, triphosphate, tetraphosphate, pentaphosphate, a 5′ cap, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, a non-bioreversible group, or a disulfide bioreversible group;
each E is independently phosphate, phosphorothioate, a non-bioreversible group, or a disulfide bioreversible group;
each F is independently H, hydroxyl, optionally substituted C₁₋₆ alkoxy, a protected hydroxyl group, a monophosphate, a diphosphate, a triphosphate, a tetraphosphate, a pentaphosphate, phosphothiol, an optionally substituted C₁₋₆ alkyl, an amino containing group, a biotin containing group, a digoxigenin containing group, a cholesterol containing group, a dye containing group, a quencher containing group, a polypeptide, a carbohydrate, a neutral organic polymer, a positively charged polymer, a therapeutic agent, a targeting moiety, an endosomal escape moiety, a non-bioreversible group, or a disulfide bioreversible group;
wherein at least one of said disulfide bioreversible groups comprises -S-S-(Link A)-B,
wherein
Link A is independently a divalent or a trivalent linker comprising sp³-hybridized carbon atom bonded to B and a carbon atom bonded to -S-S-, wherein, when Link A is a trivalent linker, the third valency of Link A combines with -S-S- to form optionally substituted C₃₋₉ heterocyclylene; and
B is independently a 5′-terminal phosphorus (V) group, a 3′-terminal phosphorus (V) group, or an internucleotide phosphorus (V) group;
wherein said hybridized polynucleotide construct comprises at least one non-bioreversible group in said guide strand, or said hybridized polynucleotide construct comprises -S-S-(Link A)-B and at least one non-bioreversible group.

4. The hybridized polynucleotide construct of claim 2, comprising at least one disulfide bioreversible group, wherein said disulfide bioreversible group has the following structure:
(R¹)_{q}-(Link C)—S—S-(Link A)-B,
wherein
each q is independently an integer from 1 to 10;
each Link C is independently a bond or a multivalent linker having a molecular weight of from 12 Da to 10000 Da; and
each R¹ is independently H, azido, a polypeptide, a carbohydrate, a neutral organic polymer, a positively charged polymer, a therapeutic agent, a targeting moiety, or an endosomal escape moiety.

5. The hybridized polynucleotide construct of claim 4, further comprising a second passenger or a second guide strand, wherein Link C is a multivalent linker further bonded to -S-S-(Link A)-B of said second passenger or said second guide strand.

6. The hybridized polynucleotide construct of claim 4, wherein Link C comprises one or more monomers, wherein each of said monomers is independently optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, wherein m is 0, 1, or 2.

7.-12. (canceled)

13. The hybridized polynucleotide construct of claim 4, wherein Link C comprises one or more poly(alkylene oxide).

14. (canceled)

15. The hybridized polynucleotide construct of claim 13, wherein said poly(alkylene oxide) is polyethylene oxide.

16. The hybridized polynucleotide construct of claim 4, wherein Link C comprises one or more groups independently selected from the group consisting of 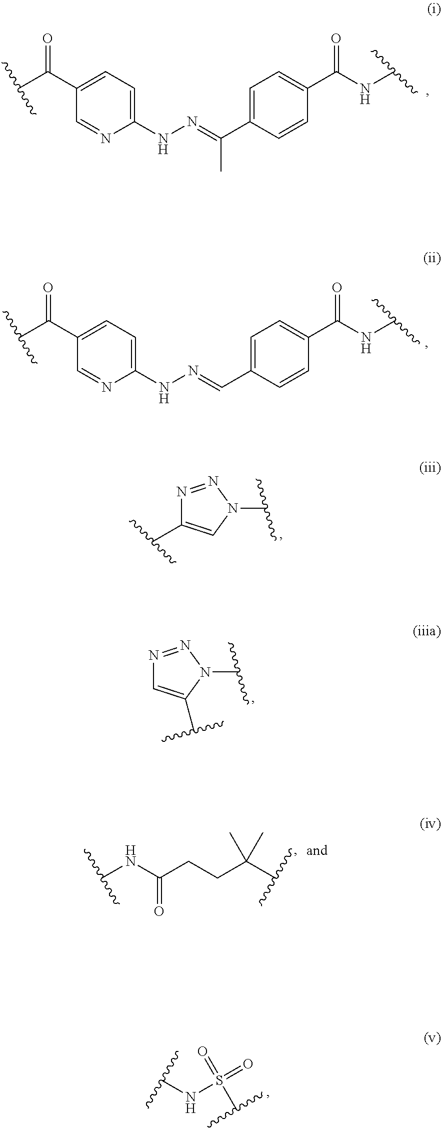
and a combination thereof.

17. The hybridized polynucleotide construct of claim 1, further comprising a second passenger strand or a second guide strand, wherein said passenger strand is linked to said second passenger strand by said non-bioreversible group, or wherein said guide-strand is linked to said second guide strand by said non-bioreversible group.

18.-20. (canceled)

21. The hybridized polynucleotide construct of claim 2, wherein Link A comprises 2 or 3 monomers, one of said monomers having the structure: 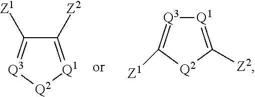
wherein
Z¹ is a bond to -S-S-;
Z² is a bond to another monomer of Link A;
Q¹ is N or CR²;
Q² is O, S, NR³, or —C(R⁵)═C(R⁶)—;
Q³ is N or C bonded to R⁴;
each of R², R³, R⁴, R⁵, and R⁶ is independently H, C₂₋₇ alkanoyl; C₁₋₆ alkyl; C₂₋₆ alkenyl; C₂₋₆ alkynyl;
C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃-cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁ aryl, and (C₆₋₁₀ aryl)-C₁₋₄-alkyl; —(CH₂)CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)SO₂R^{D} where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl; or R⁵ and R⁶, together with the atoms to which each is attached, combine to form a cyclic group selected from the group consisting of C aryl, C₂₋₇ heteroaryl, and C₂₋₇ heterocyclyl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁ aryl; amino; (C₆₋₁ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁ aryl, and (C₆₋₁ aryl)-C₁₋₄-alkyl; —(CH₂)SO₂R^{D} where q is an integer from zero to four and where R is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F} where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;
or
wherein Link A and -S-S- combine to form a structure: 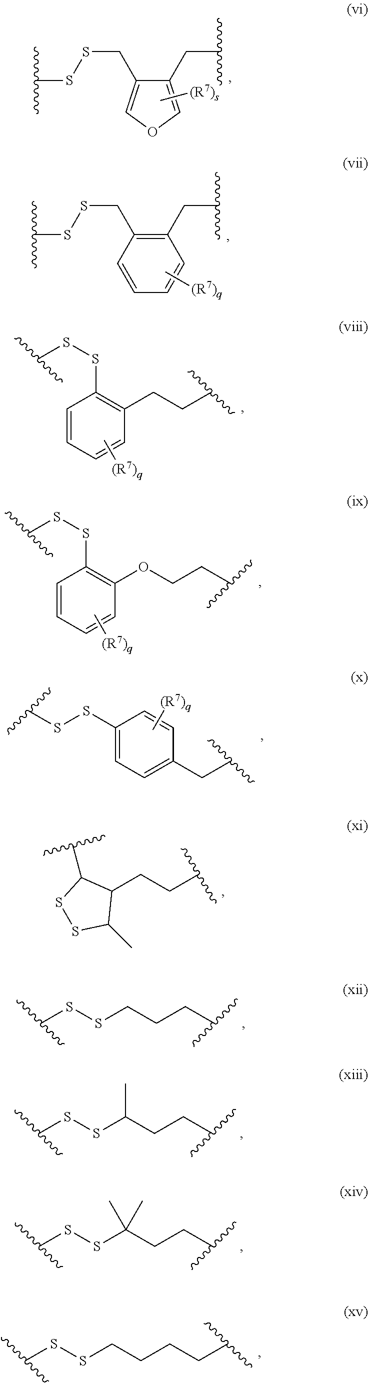 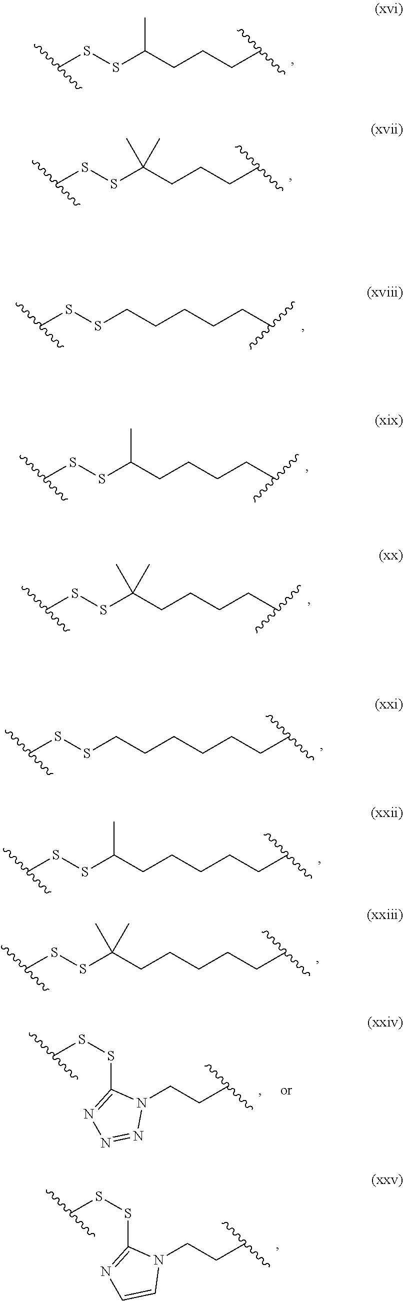
wherein
each R⁷ is independently C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl; or two adjacent R⁷ groups, together with the atoms to which each said R⁷ is attached combine to form a cyclic group selected from the group consisting of C₆ aryl, C₂₋₅ heterocyclyl, or C₂₋₅ heteroaryl, wherein said cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;
q is 0, 1, 2, 3, or 4; and
s is 0, 1, or 2;
or
wherein Link A and -S-S- combine to form a structure: 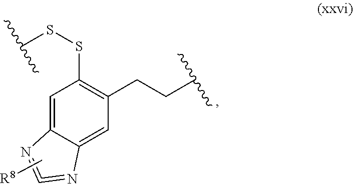
wherein the dotted lines represent one and only one double bond, and
R⁸ is attached to the nitrogen atom having a vacant valency and is H, C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl.

22.-37. (canceled)

38. The hybridized polynucleotide construct of claim 21, wherein Link A and -S-S- combine to form a structure of formula (vii), (viii), (ix), or (x), and q is 0, 1, or 2.

39.-54. (canceled)

55. The hybridized polynucleotide construct of claim 1, wherein at least one of said non-bioreversible group comprises a carbohydrate, targeting moiety, or polypeptide.

56. The hybridized polynucleotide construct of claim 1, wherein at least one of said non-bioreversible groups comprises mannose, N-acetyl galactosamine, or D-glucitol.

57.-60. (canceled)

61. The hybridized polynucleotide construct of claim 1, comprising at least one bioreversible group, wherein at least one of said bioreversible groups comprises a carbohydrate, a targeting moiety, or a polypeptide.

62. The hybridized polynucleotide construct of claim 1, comprising at least one bioreversible group, wherein at least one of said bioreversible groups comprises mannose, N-acetyl galactosamine, or D-glucitol.

63.-66. (canceled)

67. The hybridized polynucleotide construct of claim 1, wherein said guide strand comprises said non-bioreversible group.

68. The hybridized polynucleotide construct of claim 67, wherein one said non-bioreversible group connects the second nucleoside and the third nucleoside of said guide strand, the fifth nucleoside and the sixth nucleoside of said guide strand, or the seventeenth nucleoside and the eighteenth nucleoside of said guide strand.

69.-70. (canceled)

71. The hybridized polynucleotide construct of claim 67, wherein said guide strand comprises from 1 to 5 of said non-bioreversible groups.

72. (canceled)

73. The hybridized polynucleotide construct of claim 1, wherein said passenger strand comprises at least one of said non-bioreversible groups.

74. The hybridized polynucleotide construct of claim 73, wherein said non-bioreversible group connects two nucleosides of said passenger strand, wherein said nucleosides are disposed at least one nucleoside away from the natural RISC-mediated cleavage site in the 5′-direction.

75. The hybridized polynucleotide construct of claim 74, wherein said non-bioreversible group connects the first and the second nucleosides of said passenger strand.

76. The hybridized polynucleotide construct of claim 1, wherein said guide strand comprises at least one disulfide bioreversible group.

77. The hybridized polynucleotide construct of claim 76, wherein said disulfide bioreversible group connects two consecutive nucleosides selected from the three 5′-terminal or 3′-terminal nucleosides of said guide strand.

78. (canceled)

79. The hybridized polynucleotide construct of claim 1, wherein said passenger strand comprises at least one disulfide bioreversible group.

80. The hybridized polynucleotide construct of claim 79, wherein said disulfide bioreversible group connects two consecutive nucleosides selected from the three 5′-terminal or 3′-terminal nucleosides of said passenger strand.

81. (canceled)

82. The hybridized polynucleotide construct of claim 1, wherein said non-bioreversible group is a 5′-terminal group of said passenger strand or is a 3′-terminal qroup of said quide strand or said passenger strand.

83. (canceled)

84. The hybridized polynucleotide construct of claim 1, wherein said non-bioreversible group is a 3′-terminal group of said guide strand or said passenger strand.

85.-96. (canceled)

97. The hybridized polynucleotide construct of claim 1, wherein said hybridized polynucleotide comprises said disulfide bioreversible group, and the shortest chain of atoms connecting the disulfide to an internucleotide phosphorus (V) group, a 5′-terminal group, or a 3′-terminal group is 3.

98. The hybridized polynucleotide construct of claim 1, wherein said hybridized polynucleotide construct comprises said disulfide bioreversible group, and the longest chain of atoms connecting the disulfide to an internucleotide phosphorus (V) group, a 5′-terminal group, or a 3′-terminal group is 6.

99. The hybridized polynucleotide construct of claim 1, wherein said hybridized polynucleotide construct comprises said disulfide bioreversible group, and said disulfide bioreversible group comprises at least one bulky group proximal to said disulfide.

100. The hybridized polynucleotide construct of claim 1, wherein said guide strand or said passenger strand comprises 19 to 32 nucleosides.

101-107. (canceled)

108. The hybridized polynucleotide of claim 1, wherein at least one of said non-bioreversible groups is selected from the group consisting of: 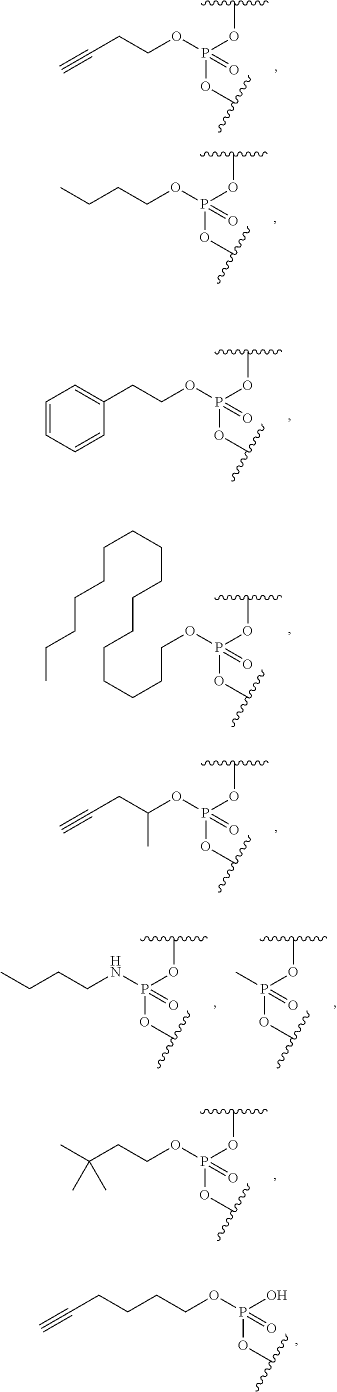 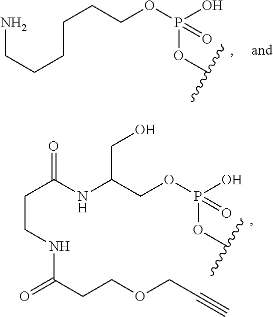 [SMILES: *OP(=O)(O)OCCCCCCN ; *OP(=O)(O)OCC(CO)NC(=O)CCNC(=O)CCOCC#C]
or a salt thereof;
or
wherein at least one of said non-bioreversible groups is formed by conjugating a polypeptide, a carbohydrate, a targeting moiety, or a delivery domain to a moiety selected from the group consisting of: 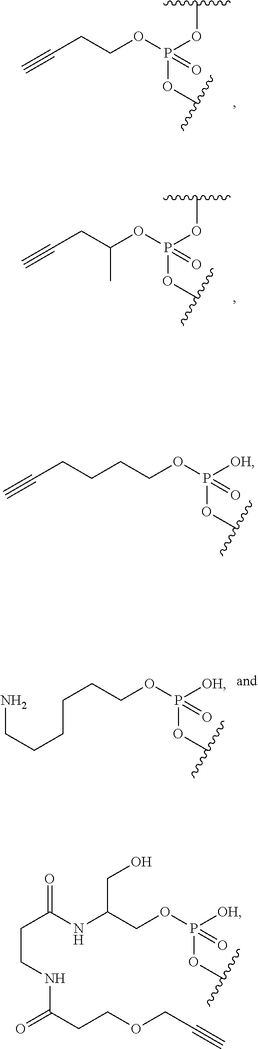 [SMILES: *OP(=O)(O*)OCCC#C ; *OP(=O)(O*)OC(C)CC#C ; *OP(=O)(O)OCCCCC#C ; *OP(=O)(O)OCCCCCCN ; *OP(=O)(O)OCC(CO)NC(=O)CCNC(=O)CCOCC#C]
or a salt thereof, wherein said moieties connect two contiguous nucleosides within or bonded to 5′-terminus of said guide strand or said passenger strand.

109. (canceled)

110. A method of delivering a polynucleotide construct to a cell comprising contacting said cell with the hybridized polynucleotide construct of claim 1, wherein, after said contacting, said polynucleotide construct resides inside said cell.

111. A method of reducing the expression of a polypeptide in a cell comprising contacting said cell with the hybridized polynucleotide construct of claim 1, wherein, after said contacting, expression of said polypeptide in said cell is reduced.


SEQUENCE LISTING (121 sequences)

SEQ ID NO: 1 - protein, 9 aa, Artificial Sequence
  Synthetic Construct
RKKRRQRRR

SEQ ID NO: 2 - protein, 8 aa, Artificial Sequence
  Synthetic Construct
RRRRRRRR

SEQ ID NO: 3 - protein, 16 aa, Artificial Sequence
  Synthetic Construct
RQIKIWFQNRRMKWKK

SEQ ID NO: 4 - protein, 21 aa, Artificial Sequence
  Synthetic Construct
TRSSRAGLQFPVGRVHRLLRK

SEQ ID NO: 5 - protein, 26 aa, Artificial Sequence
  Synthetic Construct
GWTLNSAGYLLGKINKALAALAKKIL

SEQ ID NO: 6 - protein, 18 aa, Artificial Sequence
  Synthetic Construct
KLALKLALKALKAALKLA

SEQ ID NO: 7 - protein, 16 aa, Artificial Sequence
  Synthetic Construct
AAVALLPAVLLALLAP

SEQ ID NO: 8 - protein, 10 aa, Artificial Sequence
  Synthetic Construct
VPMLKPMLKE

SEQ ID NO: 9 - protein, 28 aa, Artificial Sequence
  Synthetic Construct
MANLGYWLLALFVTMWTDVGLCKKRPKP

SEQ ID NO: 10 - protein, 18 aa, Artificial Sequence
  Synthetic Construct
LLIILRRRIRKQAHAHSK

SEQ ID NO: 11 - protein, 21 aa, Artificial Sequence
  Synthetic Construct
KETWWETWWTEWSQPKKKRKV

SEQ ID NO: 12 - protein, 18 aa, Artificial Sequence
  Synthetic Construct
RGGRLSYSRRRFSTSTGR

SEQ ID NO: 13 - protein, 15 aa, Artificial Sequence
  Synthetic Construct
SDLWEMMMVSLACQY

SEQ ID NO: 14 - protein, 12 aa, Artificial Sequence
  Synthetic Construct
TSPLNIHNGQKL

SEQ ID NO: 15 - protein, 7 aa, Artificial Sequence
  Synthetic Construct
KKRPKPG

SEQ ID NO: 16 - protein, 86 aa, Human immunodeficiency virus type 1
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 17 - protein, 130 aa, Human immunodeficiency virus type 2
METPLKEPESSLESYNEPSSCTSERDVTAQERAKQGEELLAQLHRPLEACTNSCYCKQCSYHCQLCFLKKGLGIWYARQGRRRRTPRKTKTHPPPASDKSISTRTGDSQPTKKQKKTPETTVVSACGLGH

SEQ ID NO: 18 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMSRPIIHFGSDYEDRYYRENMHRYPNQVYYRPMDEYSNQNNFVHDCVNITIKQHTVTTTTKGENFTETDVKMMERVVEQMCITQYERESQAYYKRGSSMVLFSSPPVILLISFLIFLIVG

SEQ ID NO: 19 - protein, 17 aa, Artificial Sequence
  Synthetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
GGRXRXRXRXRXRXRXR

SEQ ID NO: 20 - protein, 25 aa, Artificial Sequence
  Synthetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
  MOD_RES (18)..(18): Xaa is Boc-Lys(Fmoc)-OH
  MOD_RES (20)..(20): Xaa is Boc-Lys(Fmoc)-OH
  MOD_RES (22)..(22): Xaa is Boc-Lys(Fmoc)-OH
  MOD_RES (24)..(24): Xaa is Boc-Lys(Fmoc)-OH
GGRXRXRXRXRXRXRXRXRXRXRXR

SEQ ID NO: 21 - protein, 33 aa, Artificial Sequence
  Synthetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
  MOD_RES (18)..(18): Xaa is Boc-Lys(Fmoc)-OH
  MOD_RES (20)..(20): Xaa is Boc-Lys(Fmoc)-OH
  MOD_RES (22)..(22): Xaa is Boc-Lys(Fmoc)-OH
  MOD_RES (24)..(24): Xaa is Boc-Lys(Fmoc)-OH
  MOD_RES (26)..(26): Xaa is Boc-Lys(Fmoc)-OH
  MOD_RES (28)..(28): Xaa is Boc-Lys(Fmoc)-OH
  MOD_RES (30)..(30): Xaa is Boc-Lys(Fmoc)-OH
  MOD_RES (32)..(32): Xaa is Boc-Lys(Fmoc)-OH
GGRXRXRXRXRXRXRXRXRXRXRXRXRXRXRXR

SEQ ID NO: 22 - protein, 55 aa, Artificial Sequence
  Synthetic Construct
GGESDSELEIKRYKNRVASRKSRAKFKQLLQHYREVAAAKSSENDRLRLLLKQSS

SEQ ID NO: 23 - protein, 18 aa, Artificial Sequence
  Synthetic Construct
GGSRRHHSRSKAKRSRHH

SEQ ID NO: 24 - protein, 23 aa, Artificial Sequence
  Synthetic Construct
GGAYDLRRRERQSRLRRRERQSR

SEQ ID NO: 25 - protein, 31 aa, Artificial Sequence
  Synthetic Construct
GGMAPQRDTVGGRTTPPSWGPAKAQLRNSCA

SEQ ID NO: 26 - protein, 31 aa, Artificial Sequence
  Synthetic Construct
GGMAPQRDTVGGRTTPPSWGPAKAQLRNSSA

SEQ ID NO: 27 - protein, 8 aa, Artificial Sequence
  Synthetic Construct
GGFCIGRL

SEQ ID NO: 28 - protein, 8 aa, Artificial Sequence
  Synthetic Construct
GGGVIGRL

SEQ ID NO: 29 - protein, 17 aa, Artificial Sequence
  Synthetic Construct
GGRAWMRWYSPTTRRYG

SEQ ID NO: 30 - protein, 11 aa, Artificial Sequence
  Synthetic Construct
GGPLILLRLLR

SEQ ID NO: 31 - protein, 12 aa, Artificial Sequence
  Synthetic Construct
GGMIIYRDLISH

SEQ ID NO: 32 - protein, 13 aa, Artificial Sequence
  Synthetic Construct
GGACTGSTQHQCG

SEQ ID NO: 33 - protein, 25 aa, Artificial Sequence
  Synthetic Construct
GGALFLGWLGAAGSTMGAPKSKRKV

SEQ ID NO: 34 - protein, 25 aa, Artificial Sequence
  Synthetic Construct
GGLIRLWSHLIHIWFQNRRLKWKKK

SEQ ID NO: 35 - protein, 27 aa, Artificial Sequence
  Synthetic Construct
GGIGAVLKVLTTGLPALISWIKRKRQQ

SEQ ID NO: 36 - protein, 28 aa, Artificial Sequence
  Synthetic Construct
GGLHKLLHHLLHHLHKLLHHLHHLLHKL

SEQ ID NO: 37 - protein, 6 aa, Artificial Sequence
  Synthetic Construct
GGRKKR

SEQ ID NO: 38 - protein, 11 aa, Artificial Sequence
  Synthetic Construct
GGRKKRRQRRR

SEQ ID NO: 39 - protein, 17 aa, Artificial Sequence
  Synthetic Construct
GGRKKRRQRRRGGRKKR

SEQ ID NO: 40 - protein, 11 aa, Artificial Sequence
  Synthetic Construct
GGRKKRRQRRR

SEQ ID NO: 41 - protein, 11 aa, Artificial Sequence
  Synthetic Construct
GGRKKRRQRRR

SEQ ID NO: 42 - protein, 26 aa, Artificial Sequence
  Synthetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
  MOD_RES (18)..(18): Xaa is Boc-Lys(Fmoc)-OH
  MOD_RES (20)..(20): Xaa is Boc-Lys(Fmoc)-OH
  MOD_RES (22)..(22): Xaa is Boc-Lys(Fmoc)-OH
  MOD_RES (24)..(24): Xaa is Boc-Lys(Fmoc)-OH
GGRXRXRXRXRXRXRXRXRXRXRXRC

SEQ ID NO: 43 - protein, 26 aa, Artificial Sequence
  Sythetic Construct
  MOD_RES (4)..(4): Xaa is Boc-Lys(Fmoc)-OH
  MOD_RES (6)..(6): Xaa is Boc-Lys(Fmoc)-OH
  MOD_RES (8)..(8): Xaa is Boc-Lys(Fmoc)-OH
  MOD_RES (10)..(10): Xaa is Boc-Lys(Fmoc)-OH
  MOD_RES (12)..(12): Xaa is Boc-Lys(Fmoc)-OH
  MOD_RES (14)..(14): Xaa is Boc-Lys(Fmoc)-OH
  MOD_RES (16)..(16): Xaa is Boc-Lys(Fmoc)-OH
  MOD_RES (18)..(18): Xaa is Boc-Lys(Fmoc)-OH
  MOD_RES (20)..(20): Xaa is Boc-Lys(Fmoc)-OH
  MOD_RES (22)..(22): Xaa is Boc-Lys(Fmoc)-OH
  MOD_RES (24)..(24): Xaa is Boc-Lys(Fmoc)-OH
GGRXRXRXRXRXRXRXRXRXRXRXRC

SEQ ID NO: 44 - protein, 11 aa, Artificial Sequence
  Synthetic Construct
GGRKKRRQRRR

SEQ ID NO: 45 - protein, 28 aa, Artificial Sequence
  Synthetic Construct
GGLHKLLHHLLHHLHKLLHHLHHLLHKL

SEQ ID NO: 46 - protein, 13 aa, Artificial Sequence
  Synthetic Construct
GGACTGSTQHQCG

SEQ ID NO: 47 - protein, 25 aa, Artificial Sequence
  Synthetic Construct
GGLIRLWSHLIHIWFQNRRLKWKKK

SEQ ID NO: 48 - protein, 25 aa, Artificial Sequence
  Synthetic Construct
GGALFLGWLGAAGSTMGAPKSKRKV

SEQ ID NO: 49 - protein, 27 aa, Artificial Sequence
  Synthetic Construct
GGIGAVLKVLTTGLPALISWIKRKRQQ

SEQ ID NO: 50 - protein, 24 aa, Artificial Sequence
  Synthetic Construct
GGLFGAIAGFIENGWEGMIDGWYG

SEQ ID NO: 51 - protein, 24 aa, Artificial Sequence
  Synthetic Construct
GGLFEAIEGFIENGWEGMIDGWYG

SEQ ID NO: 52 - protein, 33 aa, Artificial Sequence
  Synthetic Construct
GGLFEAIEGFIENGWEGMIDGWYGRKKRRQRRR

SEQ ID NO: 53 - protein, 24 aa, Artificial Sequence
  Synthetic Construct
GGLFEAIEGFIENGWEGLIEGWYG

SEQ ID NO: 54 - protein, 31 aa, Artificial Sequence
  Synthetic Construct
GGKWKLFKKIGAVLKVLTTGYGRKKRRQRRR

SEQ ID NO: 55 - protein, 18 aa, Artificial Sequence
  Synthetic Construct
ILSSLTVTQLLRRLHQWI

SEQ ID NO: 56 - protein, 19 aa, Artificial Sequence
  Synthetic Construct
MKWVTFISLLFLFFSSAYS

SEQ ID NO: 57 - protein, 16 aa, Artificial Sequence
  Synthetic Construct
MIRTLLLSTLVAGALS

SEQ ID NO: 58 - protein, 18 aa, Artificial Sequence
  Synthetic Construct
RLIEDICLPRWGCLWEDD

SEQ ID NO: 59 - protein, 4 aa, Artificial Sequence
  Synthetic Construct
KDEL

SEQ ID NO: 60 - protein, 26 aa, Artificial Sequence
  Synthetic Construct
LFEAIEGFIENGWEGMIDGWYGKDEL

SEQ ID NO: 61 - protein, 35 aa, Artificial Sequence
  Synthetic Construct
LFEAIEGFIENGWEGMIDGWYGRKKRRQRRRKDEL

SEQ ID NO: 62 - protein, 18 aa, Artificial Sequence
  Synthetic Construct
RLIEDICLPRWGCLWEDD

SEQ ID NO: 63 - protein, 18 aa, Artificial Sequence
  Synthetic Construct
MKLSLVAAMLLLLSAARA

SEQ ID NO: 64 - protein, 21 aa, Artificial Sequence
  Synthetic Construct
MKLAVTLTLVTLALSSSSASA

SEQ ID NO: 65 - protein, 26 aa, Artificial Sequence
  Synthetic Construct
FFKKLAHALHLLALLALHLAHALKKA

SEQ ID NO: 66 - protein, 35 aa, Artificial Sequence
  Synthetic Construct
PSQPTYPGDDAPVRDLIRFYRDLRRYLNVVTRHRY

SEQ ID NO: 67 - protein, 22 aa, Artificial Sequence
  Synthetic Construct
RLIEDICLPRWGCLWEDDKDEL

SEQ ID NO: 68 - protein, 21 aa, Artificial Sequence
  Synthetic Construct
LFEAIEGFIENGWGMIDGWYG

SEQ ID NO: 69 - protein, 31 aa, Artificial Sequence
  Synthetic Construct
LFEAIEGFIENGWEGMIDGWYGRKKRRQRRR

SEQ ID NO: 70 - protein, 20 aa, Artificial Sequence
  Synthetic Construct
MIRTLLLSTLVAGALSKDEL

SEQ ID NO: 71 - protein, 13 aa, Artificial Sequence
  Synthetic Construct
YEQDPWGVKWWYK

SEQ ID NO: 72 - protein, 17 aa, Artificial Sequence
  Synthetic Construct
MIRTLLLSTLVAGALSK

SEQ ID NO: 73 - protein, 13 aa, Artificial Sequence
  Synthetic Construct
YEQDPWGVKWWYK

SEQ ID NO: 74 - protein, 5 aa, Artificial Sequence
  Synthetic Construct
  MOD_RES (2)..(2): Xaa is Bis-phenylalanine
  MOD_RES (4)..(4): Xaa is Bis-phenylalanine
RXRXR

SEQ ID NO: 75 - skipped

SEQ ID NO: 76 - protein, 19 aa, Artificial Sequence
  Synthetic Construct
ILSSLTVTQLLRRLHQWIK

SEQ ID NO: 77 - protein, 20 aa, Artificial Sequence
  Synthetic Construct
MIRTLLLSTLVAGALSKDEL

SEQ ID NO: 78 - protein, 35 aa, Artificial Sequence; identical to SEQ ID NO: 61
  Synthetic Construct

SEQ ID NO: 79 - protein, 25 aa, Artificial Sequence
  Synthetic Construct
IGAVLKVLTTGLPALISWIKRKRQQ

SEQ ID NO: 80 - protein, 29 aa, Artificial Sequence
  Synthetic Construct
IGAVLKVLTTGLPALISWIKRKRQQKDEL

SEQ ID NO: 81 - protein, 13 aa, Artificial Sequence
  Synthetic Construct
GGGPRRRRSSRRP

SEQ ID NO: 82 - protein, 13 aa, Artificial Sequence
  Synthetic Construct
GGGVRRRRRPRVS

SEQ ID NO: 83 - protein, 23 aa, Artificial Sequence
  Synthetic Construct
GGGPRRRRSSRRPVRRRRRPRVS

SEQ ID NO: 84 - protein, 35 aa, Artificial Sequence
  Synthetic Construct
GGGPRRRRSSRRPVRRRRRPRVSRRRRRRGGRRRR

SEQ ID NO: 85 - protein, 18 aa, Artificial Sequence
  Synthetic Construct
GGSRRHHSRSKAKRSRHH

SEQ ID NO: 86 - protein, 9 aa, Artificial Sequence
  Synthetic Construct
RRRRRRRRR

SEQ ID NO: 87 - protein, 32 aa, Artificial Sequence
  Synthetic Construct
GGWEAALAEALAEALAEHLAEALAEALEALAA

SEQ ID NO: 88 - protein, 33 aa, Artificial Sequence
  Synthetic Construct
GGWEAKLAKALAKALAKHLAKALAKALAKALLA

SEQ ID NO: 89 - protein, 5 aa, Artificial Sequence
  Synthetic Construct
RGDFK

SEQ ID NO: 90 - protein, 12 aa, Artificial Sequence
  Synthetic Construct
ERGDFKERGDFK

SEQ ID NO: 91 - protein, 9 aa, Artificial Sequence
  Synthetic Construct
CRGDRGPDC

SEQ ID NO: 92 - protein, 9 aa, Artificial Sequence
  Synthetic Construct
CRGDKGPDC

SEQ ID NO: 93 - protein, 9 aa, Artificial Sequence
  Synthetic Construct
CRNDRGPDC

SEQ ID NO: 94 - protein, 29 aa, Artificial Sequence
  Synthetic Construct
YTIWMPENPRPGTPCDIFTNSRGKRASNG

SEQ ID NO: 95 - protein, 35 aa, Artificial Sequence
  Synthetic Construct
YTSLIHSLIEESQNQQEKQEKELMELERWGSMLKC

SEQ ID NO: 96 - protein, 35 aa, Artificial Sequence
  Synthetic Construct
YTSLIHSLIEESQNQQEKQEKELMELERWGSMLQL

SEQ ID NO: 97 - protein, 37 aa, Artificial Sequence
  Synthetic Construct
GGPSQPTYPGDDAPVRDLIRFYRDLRRYLNVVTRHRY

SEQ ID NO: 98 - protein, 6 aa, Artificial Sequence
  Synthetic Construct
AAKDEL

SEQ ID NO: 99 - protein, 11 aa, Artificial Sequence
  Synthetic Construct
GEEDTSEKDEL

SEQ ID NO: 100 - protein, 12 aa, Artificial Sequence
  Synthetic Construct
ASQPGKPPKDEL

SEQ ID NO: 101 - protein, 12 aa, Artificial Sequence
  Synthetic Construct
ASQPGKPPREDL

SEQ ID NO: 102 - protein, 16 aa, Artificial Sequence
  Synthetic Construct
GRQSDIDTHNRIKDEL

SEQ ID NO: 103-105 - skipped (3 entries)

SEQ ID NO: 106 - protein, 5 aa, Artificial Sequence
  Synthetic Construct
GGGGS

SEQ ID NO: 107 - protein, 12 aa, Artificial Sequence
  Synthetic Construct
GKSSGSGSESKS

SEQ ID NO: 108 - protein, 14 aa, Artificial Sequence
  Synthetic Construct
GSTSGSGKSSEGKG

SEQ ID NO: 109 - protein, 18 aa, Artificial Sequence
  Synthetic Construct
GSTSGSGKSSEGSGSTKG

SEQ ID NO: 110 - protein, 18 aa, Artificial Sequence
  Synthetic Construct
GSTSGSGKPGSGEGSTKG

SEQ ID NO: 111 - protein, 14 aa, Artificial Sequence
  Synthetic Construct
EGKSSGSGSESKEF

SEQ ID NO: 112 - DNA, 21 bp, Artificial Sequence
  Synthetic Construct
gcuacauucuggagacauaut

SEQ ID NO: 113 - DNA, 21 bp, Artificial Sequence
  Synthetic Construct
uaugucuccagaauguagcut

SEQ ID NO: 114 - DNA, 21 bp, Artificial Sequence
  Synthetic Construct
ucuacauguuccaguaugaut

SEQ ID NO: 115 - DNA, 21 bp, Artificial Sequence
  Synthetic Construct
ucauacuggaacauguagaut

SEQ ID NO: 116 - DNA, 21 bp, Artificial Sequence
  Synthetic Construct
ucaucacacugaauaccaaut

SEQ ID NO: 117 - DNA, 21 bp, Artificial Sequence
  Synthetic Construct
uugguauucagugugaugaut

SEQ ID NO: 118 - DNA, 21 bp, Artificial Sequence
  Synthetic Construct
aguacugcuuacgauacggut

SEQ ID NO: 119 - DNA, 21 bp, Artificial Sequence
  Synthetic Construct
ccguaucguaagcaguacuut

SEQ ID NO: 120 - DNA, 21 bp, Artificial Sequence
  Synthetic Construct
aggaucaucucaagucuuaut

SEQ ID NO: 121 - DNA, 21 bp, Artificial Sequence
  Synthetic Construct
uaagacuugagaugauccuut